SEQUENCE LISTING (113 sequences)

SEQ ID NO: 1 - RNA, 59 nt, Artificial Sequence
  Chemically Synthesized
ucuccucgaggggucucugccucuacccaggacucuuucaugaccaggaggcugaggcc

SEQ ID NO: 2 - RNA, 30 nt, Artificial Sequence
  Chemically Synthesized
cugaggagguccucacagaagaagaagggu

SEQ ID NO: 3 - RNA, 32 nt, Artificial Sequence
  Chemically Synthesized
ggcugcggaggugcucacagaagagcaagggu

SEQ ID NO: 4 - RNA, 56 nt, Artificial Sequence
  Chemically Synthesized
gugccugccugugagcgccucgacgacagagccggcgccugccccagugucugcgc

SEQ ID NO: 5 - RNA, 30 nt, Artificial Sequence; identical to SEQ ID NO: 2
  Chemically Synthesized

SEQ ID NO: 6 - RNA, 23 nt, Artificial Sequence
  Chemically Synthesized
ucccuguccuccaggagcucacg

SEQ ID NO: 7 - RNA, 29 nt, Artificial Sequence
  Chemically Synthesized
ucuucucggagcugcuguccaaccuguac

SEQ ID NO: 8 - RNA, 19 nt, Artificial Sequence
  Chemically Synthesized
cgaggacagguugaccgag

SEQ ID NO: 9 - RNA, 29 nt, Artificial Sequence
  Chemically Synthesized
ugcaaauggaagcgauuagaaacuagguc

SEQ ID NO: 10 - RNA, 22 nt, Artificial Sequence
  Chemically Synthesized
aggucguuucccuucgcgaaag

SEQ ID NO: 11 - RNA, 29 nt, Artificial Sequence
  Chemically Synthesized
ugcaaauggaagcgauuagaaacuagguc

SEQ ID NO: 12 - RNA, 21 nt, Artificial Sequence
  Chemically Synthesized
cgagguuucccuucgcgaaac

SEQ ID NO: 13 - RNA, 29 nt, Artificial Sequence
  Chemically Synthesized
uagaguugaaagggaaacgucuuugaggu

SEQ ID NO: 14 - RNA, 21 nt, Artificial Sequence
  Chemically Synthesized
cgagguuucccuucgcgaaac

SEQ ID NO: 15 - RNA, 29 nt, Artificial Sequence
  Chemically Synthesized
caggaggcaaaggguaagugguuucauuc

SEQ ID NO: 16 - RNA, 22 nt, Artificial Sequence
  Chemically Synthesized
aggucguuucccuucgcgaaag

SEQ ID NO: 17 - RNA, 29 nt, Artificial Sequence
  Chemically Synthesized
uuguuuucuuuuagaaagucucccuguuu

SEQ ID NO: 18 - RNA, 29 nt, Artificial Sequence
  Chemically Synthesized
uuguuuugucugagaaaaucucgcuguuu

SEQ ID NO: 19 - RNA, 22 nt, Artificial Sequence
  Chemically Synthesized
aguguucaaucccagagucccu

SEQ ID NO: 20 - DNA, 21 bp, Artificial Sequence
  Chemically Synthesized
ttgcagttggactctcaggga

SEQ ID NO: 21 - RNA, 22 nt, Artificial Sequence
  Chemically Synthesized
aguguucaaucccagagucccu

SEQ ID NO: 22 - DNA, 22 bp, Artificial Sequence
  Chemically Synthesized
gcacagcctagggtcttggggg

SEQ ID NO: 23 - RNA, 52 nt, Artificial Sequence
  Chemically Synthesized
auuaugaagguuaaaucauacaauauaauguuuaauaaauauuaacaauuau

SEQ ID NO: 24 - RNA, 21 nt, Artificial Sequence
  Chemically Synthesized
cuauacaaucuacugucuuuc

SEQ ID NO: 25 - RNA, 52 nt, Artificial Sequence
  Chemically Synthesized
auuaugaagguuaaaucauacaauacaauguuuaauaaauauuaacaauuau

SEQ ID NO: 26 - RNA, 21 nt, Artificial Sequence
  Chemically Synthesized
cuauacaaucuacugucuuuc

SEQ ID NO: 27 - RNA, 52 nt, Artificial Sequence
  Chemically Synthesized
gaaauuucaacugggcugccucuguccuugggcauaugccuccuccuccguc

SEQ ID NO: 28 - RNA, 21 nt, Artificial Sequence
  Chemically Synthesized
gcaguccaugggcauauacac

SEQ ID NO: 29 - RNA, 52 nt, Artificial Sequence
  Chemically Synthesized
gaaauuucaacugggcugccucugucuuugggcauaugccuccuccuccguc

SEQ ID NO: 30 - RNA, 21 nt, Artificial Sequence
  Chemically Synthesized
gcaguccaugggcauauacac

SEQ ID NO: 31 - RNA, 52 nt, Artificial Sequence
  Chemically Synthesized
auuuugcuaaauaaguauauauuaaacaaagguccauaaccaccauacacau

SEQ ID NO: 32 - RNA, 21 nt, Artificial Sequence
  Chemically Synthesized
uaaaguaaauaugcaccaaaa

SEQ ID NO: 33 - RNA, 52 nt, Artificial Sequence
  Chemically Synthesized
auuuugcuaaauaaguauauauuaaauaaagguccauaaccaccauaucacu

SEQ ID NO: 34 - RNA, 21 nt, Artificial Sequence
  Chemically Synthesized
uaaaguaaauaugcaccaaaa

SEQ ID NO: 35 - RNA, 52 nt, Artificial Sequence
  Chemically Synthesized
acaaaggaggguagugguacaaggggugagaagaguuggauuccagcuaguu

SEQ ID NO: 36 - RNA, 21 nt, Artificial Sequence
  Chemically Synthesized
uuggccacaauggguuagaac

SEQ ID NO: 37 - RNA, 52 nt, Artificial Sequence
  Chemically Synthesized
acaaaggaggguagugguacaagggcugagaagaguuggauuccagcuaguu

SEQ ID NO: 38 - RNA, 21 nt, Artificial Sequence
  Chemically Synthesized
uuggccacaauggguuagaac

SEQ ID NO: 39 - RNA, 52 nt, Artificial Sequence
  Chemically Synthesized
gcauguuuggguuuuaauaauagaauuuuaaaagacugguagaucacuugug

SEQ ID NO: 40 - RNA, 23 nt, Artificial Sequence
  Chemically Synthesized
cagugcaauaguauugucaaagc

SEQ ID NO: 41 - RNA, 52 nt, Artificial Sequence
  Chemically Synthesized
gcauguuuggguuuuaauaauagaacuuuaaaagacugguagaucacuugug

SEQ ID NO: 42 - RNA, 23 nt, Artificial Sequence
  Chemically Synthesized
cagugcaauaguauugucaaagc

SEQ ID NO: 43 - RNA, 52 nt, Artificial Sequence; identical to SEQ ID NO: 41
  Chemically Synthesized

SEQ ID NO: 44 - RNA, 22 nt, Artificial Sequence
  Chemically Synthesized
uuauaauacaaccugauaagug

SEQ ID NO: 45 - RNA, 52 nt, Artificial Sequence; identical to SEQ ID NO: 39
  Chemically Synthesized

SEQ ID NO: 46 - RNA, 22 nt, Artificial Sequence
  Chemically Synthesized
uuauaauacaaccugauaagug

SEQ ID NO: 47 - RNA, 52 nt, Artificial Sequence; identical to SEQ ID NO: 41
  Chemically Synthesized

SEQ ID NO: 48 - RNA, 22 nt, Artificial Sequence
  Chemically Synthesized
cagugcaauguuaaaagggcau

SEQ ID NO: 49 - RNA, 52 nt, Artificial Sequence; identical to SEQ ID NO: 39
  Chemically Synthesized

SEQ ID NO: 50 - RNA, 22 nt, Artificial Sequence
  Chemically Synthesized
cagugcaauguuaaaagggcau

SEQ ID NO: 51 - RNA, 52 nt, Artificial Sequence
  Chemically Synthesized
gcaugccaguugcccacuguggcaaucaauaucugagccugugguuuuugcc

SEQ ID NO: 52 - RNA, 72 nt, Artificial Sequence
  Chemically Synthesized
guaguuugugcuguuggucggguugugacauugcccgcuguggagauaacugcgcaagcuacugccuugcua

SEQ ID NO: 53 - RNA, 52 nt, Artificial Sequence
  Chemically Synthesized
gcaugccaguugcccacuguggcaauaaauaucugagccugugguuuuugcc

SEQ ID NO: 54 - RNA, 72 nt, Artificial Sequence; identical to SEQ ID NO: 52
  Chemically Synthesized

SEQ ID NO: 55 - RNA, 52 nt, Artificial Sequence
  Chemically Synthesized
aacagagacuauccaagugcaucacuaaauucacagcagucgcaaggaaccu

SEQ ID NO: 56 - RNA, 22 nt, Artificial Sequence
  Chemically Synthesized
caaucacuaacuccacugccau

SEQ ID NO: 57 - RNA, 52 nt, Artificial Sequence
  Chemically Synthesized
aacagagacuauccaagugcaucacaaaauucacagcagucgcaaggaaccu

SEQ ID NO: 58 - RNA, 22 nt, Artificial Sequence
  Chemically Synthesized
caaucacuaacuccacugccau

SEQ ID NO: 59 - RNA, 52 nt, Artificial Sequence
  Chemically Synthesized
cacccuucugauaaaucuuuguuuguucaugcuuuguuugguaagagucagu

SEQ ID NO: 60 - RNA, 22 nt, Artificial Sequence
  Chemically Synthesized
cgaaucauuauuugcugcucua

SEQ ID NO: 61 - RNA, 52 nt, Artificial Sequence
  Chemically Synthesized
cacccuucugauaaaucuuuguuugcucaugcuuuguuugguaagagucagu

SEQ ID NO: 62 - RNA, 22 nt, Artificial Sequence
  Chemically Synthesized
cgaaucauuauuugcugcucua

SEQ ID NO: 63 - RNA, 52 nt, Artificial Sequence
  Chemically Synthesized
ugaagcuuuucuugucauuuccaucaggauacaaucucuccugcuuuaaacu

SEQ ID NO: 64 - RNA, 21 nt, Artificial Sequence
  Chemically Synthesized
cuccagagggaugcacuuucu

SEQ ID NO: 65 - RNA, 52 nt, Artificial Sequence
  Chemically Synthesized
ugaagcuuuucuugucauuuccaucgggauacaaucucuccugcuuuaaacu

SEQ ID NO: 66 - RNA, 21 nt, Artificial Sequence
  Chemically Synthesized
cuccagagggaugcacuuucu

SEQ ID NO: 67 - DNA, 52 bp, Artificial Sequence
  Chemically Synthesized
attcctgaataaacattgttttcttttagaaagtctccctgtttgttattta

SEQ ID NO: 68 - RNA, 29 nt, Artificial Sequence
  Chemically Synthesized
uuguuuucuuuuagaaagucucccuguuu

SEQ ID NO: 69 - RNA, 29 nt, Artificial Sequence
  Chemically Synthesized
uuguuuugucugagaaaaucucgcuguuu

SEQ ID NO: 70 - RNA, 30 nt, Artificial Sequence
  Chemically Synthesized
ggguucgccgcccucccuugccucccucuu

SEQ ID NO: 71 - RNA, 30 nt, Artificial Sequence
  Chemically Synthesized
gggugcgccgcccucccuugccucccucuu

SEQ ID NO: 72 - RNA, 22 nt, Artificial Sequence
  Chemically Synthesized
aguguucaaucccagagucccu

SEQ ID NO: 73 - DNA, 21 bp, Artificial Sequence
  Chemically Synthesized
ttgcagttggactctcaggga

SEQ ID NO: 74 - RNA, 22 nt, Artificial Sequence
  Chemically Synthesized
aguguucaaucccagagucccu

SEQ ID NO: 75 - DNA, 22 bp, Artificial Sequence
  Chemically Synthesized
gcacagcctagggtcttggggg

SEQ ID NO: 76 - RNA, 17 nt, Artificial Sequence
  Chemically Synthesized
ugggagacuuuccuucg

SEQ ID NO: 77 - DNA, 17 bp, Artificial Sequence
  Chemically Synthesized
acccattattgggaagc

SEQ ID NO: 78 - RNA, 19 nt, Artificial Sequence
  Chemically Synthesized
ugucuaucagauuugugac

SEQ ID NO: 79 - DNA, 19 bp, Artificial Sequence
  Chemically Synthesized
aaacatattctgaacattg

SEQ ID NO: 80 - RNA, 22 nt, Artificial Sequence
  Chemically Synthesized
cuugucuaucagauuugugacc

SEQ ID NO: 81 - DNA, 19 bp, Artificial Sequence
  Chemically Synthesized
gatttgatgatgacactgg

SEQ ID NO: 82 - RNA, 10 nt, Artificial Sequence
  Chemically Synthesized
aauaggauuu

SEQ ID NO: 83 - DNA, 10 bp, Artificial Sequence
  Chemically Synthesized
ttattctaaa

SEQ ID NO: 84 - RNA, 18 nt, Artificial Sequence
  Chemically Synthesized
ucaguucgaauaggauuu

SEQ ID NO: 85 - DNA, 18 bp, Artificial Sequence
  Chemically Synthesized
attcaaacatattctgaa

SEQ ID NO: 86 - RNA, 21 nt, Artificial Sequence
  Chemically Synthesized
uugucuaucagauuugugacc

SEQ ID NO: 87 - DNA, 21 bp, Artificial Sequence
  Chemically Synthesized
ggcaaggggtgggcacattgg

SEQ ID NO: 88 - RNA, 21 nt, Artificial Sequence
  Chemically Synthesized
acucgaggaccuccugucccu

SEQ ID NO: 89 - DNA, 21 bp, Artificial Sequence
  Chemically Synthesized
tgggagcatggcagacaggga

SEQ ID NO: 90 - RNA, 21 nt, Artificial Sequence
  Chemically Synthesized
ucucccucuggguccgagccu

SEQ ID NO: 91 - DNA, 22 bp, Artificial Sequence
  Chemically Synthesized
acccggagactgtgggctcggg

SEQ ID NO: 92 - RNA, 22 nt, Artificial Sequence
  Chemically Synthesized
ccucacuucugugccucggucu

SEQ ID NO: 93 - DNA, 22 bp, Artificial Sequence
  Chemically Synthesized
gaaccccgggttcagggccagg

SEQ ID NO: 94 - RNA, 19 nt, Artificial Sequence
  Chemically Synthesized
gacuccagacccgguccag

SEQ ID NO: 95 - DNA, 19 bp, Artificial Sequence
  Chemically Synthesized
ccgggttcagggccagggc

SEQ ID NO: 96 - RNA, 18 nt, Artificial Sequence
  Chemically Synthesized
guaccucaggagguccaa

SEQ ID NO: 97 - DNA, 17 bp, Artificial Sequence
  Chemically Synthesized
tgtgggaaccccgggtt

SEQ ID NO: 98 - RNA, 21 nt, Artificial Sequence
  Chemically Synthesized
cgacucuugggucuccagagu

SEQ ID NO: 99 - DNA, 21 bp, Artificial Sequence
  Chemically Synthesized
gccctggtcctgggggactca

SEQ ID NO: 100 - RNA, 17 nt, Artificial Sequence
  Chemically Synthesized
cuccacacccgguccag

SEQ ID NO: 101 - DNA, 17 bp, Artificial Sequence
  Chemically Synthesized
gagctccgagccaggtc

SEQ ID NO: 102 - RNA, 21 nt, Artificial Sequence
  Chemically Synthesized
ucguaccucaggagguccaac

SEQ ID NO: 103 - DNA, 22 bp, Artificial Sequence
  Chemically Synthesized
ggcattggtgacgtccaggttg

SEQ ID NO: 104 - RNA, 20 nt, Artificial Sequence
  Chemically Synthesized
gagucuugggucuccagagu

SEQ ID NO: 105 - DNA, 22 bp, Artificial Sequence
  Chemically Synthesized
ttcagaagcaagtcaggtttca

SEQ ID NO: 106 - RNA, 22 nt, Artificial Sequence
  Chemically Synthesized
uugugagauuucccuacgugca

SEQ ID NO: 107 - DNA, 22 bp, Artificial Sequence
  Chemically Synthesized
aactttttaaagaagtgcatga

SEQ ID NO: 108 - RNA, 22 nt, Artificial Sequence
  Chemically Synthesized
ugugagauuuuccuacgugcua

SEQ ID NO: 109 - DNA, 22 bp, Artificial Sequence
  Chemically Synthesized
actttttaaagaagtgcatgaa

SEQ ID NO: 110 - RNA, 22 nt, Artificial Sequence
  Chemically Synthesized
uugugagauuucccuacgugca

SEQ ID NO: 111 - DNA, 20 bp, Artificial Sequence
  Chemically Synthesized
gaagttctcatggtgcacga

SEQ ID NO: 112 - RNA, 22 nt, Artificial Sequence
  Chemically Synthesized
ugugagauuuuccuacgugcua

SEQ ID NO: 113 - DNA, 20 bp, Artificial Sequence
  Chemically Synthesized
aagttctcatggtgcacgag

(19) United States
(12) Patent Application Publication
(10) Pub. No.: US 2010/0130526 A1
(43) Pub. Date: May 27, 2010

(54) Methods for Disease Therapy

(76) Inventor: Gennadi V. Glinsky, Loudonville, NY (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/476,092

(22) Filed: Jun. 1, 2009

Related U.S. Application Data
(60) Provisional application No. 61/057,428, filed on May 30, 2008.
(60) Provisional application No. 61/086,667, filed on Aug. 6, 2008.
(60) Provisional application No. 61/111,069, filed on Nov. 4, 2008.
(60) Provisional application No. 61/118,924, filed on Dec. 1, 2008.

(51) Int. Cl. A61K 31/437; C12Q 1/68; A61K 31/47; C12M 1/34

(52) U.S. Cl. 514/291; 435/6; 514/313; 435/287.2

(57) ABSTRACT

The present invention discloses disease-linked SNPs, microRNAs, and microRNA-targeted mRNAs relevant to the pathogenesis of several major human disorders including, but not limited to, multiple types of cancers, type 2 diabetes, type 1 diabetes, Crohn's disease, coronary artery disease, hypertension, rheumatoid arthritis, bipolar disorder. Also provided are methods for the identification of disease phenotype-defining sets of SNPs, microRNAs, and mRNAs that are defined here as a “consensus disease phenocode” as well as methods of using the information provided by these consensus disease phenocodes for various diagnostic, prognostic, and/or therapeutic applications.

### RELATED APPLICATIONS

This application claims priority to U.S. Provisional Application 61/057,428, filed May 30, 2008, U.S. Provisional Application 61/086,667, filed on Aug. 6, 2008, U.S. Provisional Application 61/111,069, filed on Nov. 4, 2008 and to U.S. Provisional Application 61/118,924, filed on Dec. 1, 2008, all of which are incorporated by reference in their entireties.

### FIELD OF THE INVENTION

The present invention relates generally to disease-linked SNPs, microRNAs, and microRNA-targeted mRNAs.

### BACKGROUND

Recently, knowledge of a genomic universe of human transcriptome dramatically expanded. It unravels remarkable quantitative and qualitative diversities of structural, functional, and regulatory features of the human genome. It is now understood that the human genome, in addition to mRNAs encoded by some 22,000 protein-coding genes, generates hundreds thousands (perhaps, millions) of transcripts with limited or no protein-coding potentials. A remarkable diversity of RNA species of the human transcriptome may contribute to the dramatic increase of regulatory complexity and phenotypic evolution of Homo sapiens despite having similar numbers of the protein coding genes compared to other eukaryotes. However, the critical missing link of this attractive hypothesis is the lack of conceptual or experimental evidence supporting the notion that most short non-coding RNAs (sncRNAs) contribute to phenotypes.

### SUMMARY OF THE INVENTION

The present invention provides methods of identifying a phenotype-linked variant genomic sequence in an individual by providing a genomic sequence, where the genomic sequence is associated with a disease or condition and contains a known sequence variation; assessing expression of the genomic sequence; and correlating the genomic sequence and expression to identify a variant genomic sequence whose expression is altered in a subject with a disease or condition, thereby identifying a phenotype-linked variant genomic sequence. In various embodiments, the genomic sequence is a single-nucleotide polymorphism (SNP); a copy number variation (CNV) loss of heterozygocity (LOH); amplification; deletions; insertions; point mutations; frame-shift; duplication; and/or epigenetic sequence modifications such as DNA methylation; epigenetic silencing or activation of transcription such as modification of histone codes and nucleosomes. Those skilled in the art will recognize that the altered sequence expression is either an increase in expression or a decrease in expression as compared to subject not having the disease or condition.

As provided are methods for displaying, recording, or communicating the identified phenotype-linked variant genomic sequence. The information can be displayed, for example, as a two and/or three dimensional plot, a cascade flowchart, or other two or three pictorial representation of the molecular pathways and elements thereof, on a display device. Display devices include, but are not limited to, computer monitors (e.g., via the INTERNET or INTRANET), television screens, hand-held devices, and the like. Provision of such information can be interactive as for example on a computer screen, printed, or otherwise displayed.

The present invention provides not only a method, system and program for creating and using a phenocode, but also a recording medium in which the phenocode and uses thereof are recorded. The recording medium may be computer-readable. Examples of the medium include a floppy disc (FD), a magneto-optical disc (MO), a CD-ROM, a hard disc, a ROM and a RAM.

The methods of identifying a phenotype-linked variant genomic sequence in an individual provided herein further involve the steps of building a map of the identified phenotype-linked variant genomic sequence; using the identified phenotype-linked variant genomic sequence to identify gene expression signatures with respect to the phenotype-linked variant genomic sequence; and selecting the phenotype-linked variant genomic sequence by cross referencing the gene expression signatures to the map of the identified phenotype-linked variant genomic sequence.

In another aspect, the invention provides methods of identifying a phenocode by, querying a microRNA database with a variant genomic sequence whose expression is altered in a subject with a disease or condition, thereby identifying a microRNA homologous to the variant genomic sequence and identifying an mRNA homologous to the microRNA, thereby identifying a phenocode comprising the variant genomic sequence, the homologous microRNA, and the mRNA. Again the genomic sequence maybe, for example, a single-nucleotide polymorphism (SNP) or a copy number variation (CNV). Those skilled in the art will recognize that other genomic sequences can also be used. The method of identifying a phenocode further also can involve the steps of displaying the phenocode and/or producing a sequence homology map. In one embodiment, the variant genomic sequence is the top scoring variant genomic sequence and wherein the method further involves the step of identifying microRNAs having largest number of homology events.

Those skilled in the art will recognize that, in one embodiment, the identified microRNA is homologous to the variant genomic sequence whose expression is altered in the subject with the disease or condition. Preferably, the identified microRNA targets one or more protein-coding mRNAs, for example, protein-coding mRNAs in the nuclear import pathway or the inflammasome pathway.

Generally, the diseases or conditions include, but are not limited to, breast cancer, prostate cancer, colorectal cancer, lung cancer, ovarian cancer, systemic lupus erythematosus, vitiligo, vitiligo-associated multiple autoimmune disease, type 2 diabetes, type 1 diabetes, Crohn's disease, coronary artery disease, hypertension, rheumatoid arthritis, bipolar disorder, ankylosing spondylitis, Graves' disease, multiple sclerosis, Huntington's disease, ulcerative colitis, Alzheimer's, autism, autoimmune thyroid disease, schizophrenia, ageing and centenarians phenotypes.

These methods of identifying a phenocode also involves the step of identifying those mRNAs that are encoded by protein-coding genes and assessing the expression of the identified mRNAs. For example, the protein-coding gene is part of the nuclear import pathway or the inflammasome pathway. Examples of such protein-coding genes include, but are not limited to, KPNA1, NLRP1, NLRP3, HLA-DRB1, PTPN22, OLIG3/TNFAIP3, STAT4, TRAF1/C5, and any combination(s) thereof. More particularly, genes comprising the ten-gene Crohn's disease signature are: ACAN; WNT5A; MMP14; HOXA11; EN1; DICER1; TSC1; MYB; MYBL1; HMGA1. Further, the genes comprising the ten-gene rheumatoid arthritis signature are: ACAN; WNT5A; MMP14; HOXA11; CEBPB; DICER1; TSC1; MYB; MYBL1; PTEN. More particularly, the protein-coding gene is KPNA1, and the expression of KPNA1 is altered in the disease or condition.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected.

An exemplary method for detecting the presence or absence of a protein or nucleic acid (e.g., mRNA, genomic DNA) in a biological sample involves obtaining a biological sample from a test subject and contacting the biological sample with a compound or an agent capable of detecting protein or nucleic acid that encodes a protein such that the presence of the protein is detected in the biological sample. An agent for detecting mRNA or genomic DNA is a labeled nucleic acid probe capable of hybridizing to mRNA or genomic DNA. The nucleic acid probe can be, for example, a full-length nucleic acid, such as an oligonucleotide of at least 15, 30, 50, 100, 250 or 500 nucleotides in length and sufficient to specifically hybridize under stringent conditions to mRNA or genomic DNA.

The invention further includes methods for detecting or diagnosing the presence of a disease associated with altered levels of a nucleic acid in a sample from a mammal, e.g. a human. For example, such methods include measuring the level of the nucleic acid in a biological sample from the mammalian subject and comparing the level detected to a level of the nucleic acid present in normal subjects, or in the same subject at a different time. An increase or decrease in the level of the nucleic acid as compared to normal levels indicates a disease condition.

These methods may further involve obtaining a control biological sample from a control subject, contacting the control sample with a compound or agent capable of detecting a protein, mRNA, or genomic DNA, such that the presence of a protein, mRNA or genomic DNA is detected in the biological sample, and comparing the presence of a protein, mRNA or genomic DNA in the control sample with the presence of a protein, mRNA or genomic DNA in the test sample.

In another aspect, a computer-readable medium comprising computer executable instructions recorded thereon is utilized for performing the method comprising querying a microRNA database with a variant genomic sequence whose expression is altered in a subject with a disease or condition to identify a microRNA homologous to the variant genomic sequence. The method further includes identifying an mRNA homologous to the microRNA, thereby obtaining a phenocode comprising said variant genomic sequence, the homologous microRNA, and said mRNA and displaying said phenocode on the computer-readable medium.

Also provided are methods of reversing a disease or condition associated with altered gene expression phenotypes of the nuclear import or inflammasome pathways comprising administering an effective amount of a pharmaceutical compound to a subject. By way of non-limiting example, the pharmaceutical compound can be chloroquine or rapamycin. Following administration of the pharmaceutical compound, the alteration of gene expression is reversed in the subject. For example, the gene whose expression is altered, may include, but it not limited to, one or more of the KPNA1, NLRP1, and NLRP3 genes.

The invention also provides an apparatus for evaluating a disease or a risk of disease in a patient, comprising a model predictive of a disease phenocode configured to evaluate a dataset for patient to thereby evaluate the risk of disease in said patient, wherein the model is based on a set of disease-linked SNPs, microRNAs displaying sequence homology or complementarity to the disease-linked SNPs, and mRNAs encoded by protein-coding genes, wherein said mRNAs are targeted by said microRNAs, wherein the disease-linked SNPs exert a regulatory effect in trans.

For example, the apparatus can be used to evaluate a disease or a risk of disease, including by not limited to, breast cancer, prostate cancer, systemic lupus erythematosus, vitiligo-associated multiple autoimmune disease, type 2 diabetes, type 1 diabetes, Crohn's disease, coronary artery disease, hypertension, rheumatoid arthritis, bipolar disorder, ankylosing spondylitis, Graves' disease, multiple sclerosis, Huntington's disease, and ulcerative colitis.

The present invention also includes consensus disease phenocodes comprising a set of disease-linked SNPs, microRNAs displaying sequence homology or complementarity to the disease-linked SNPs, and mRNAs encoded by protein-coding genes, wherein the mRNAs are targeted by the microRNAs, and wherein the disease-linked SNPs exert a regulatory effect in trans. Those skilled in the art will recognize that the information provided by such phenocodes can be utilized in a variety of ways. For example, for genetic counseling, for screening for treatments, for assessment of treatment efficacy, for diagnosis of a disease or condition, etc. The present invention also includes systems for evaluating a disease or risk of disease in a patient, which involves evaluating the patient for a set of disease-linked SNPs, microRNAs displaying sequence homology or complementarity to the disease-linked SNPs, and mRNAs encoded by protein-coding genes, wherein said mRNAs are targeted by said microRNAs, and wherein the disease-linked SNPs exert a regulatory effect in trans.

The present invention also includes methods of screening for candidate compounds capable of reversing a disease or condition associated with an altered gene expression phenotypes of the nuclear import or inflammasome pathways by: detecting the level of gene expression in a subject administered a candidate compound, wherein the subject is suffering from a disease or condition; comparing the level of gene expression for the candidate compound with that of a reference compound known to reverse the altered gene expression associated with the disease or condition; and determining the differences, if any, between the levels of gene expression for the candidate compound and the reference compound, thereby identifying whether the candidate compound is capable of reversing the disease or condition. By way of non-limiting example, the reference compound may be chloroquine or rapamycin.

Also provides are methods of determining susceptibility to a disease or condition in a subject, the method comprising determining for said subject a disease phenocode, wherein said phenocode comprises (i) a set of disease-linked SNPs, (ii) microRNAs displaying sequence homology or complementarity to the disease-linked SNPs, and (iii) mRNAs encoded by protein-coding genes, wherein the mRNAs are targeted by the microRNAs, and wherein the disease-linked SNPs exert a regulatory effect in trans; and assessing susceptibility to the disease in the subject based on the phenocode.

In another aspect, the invention provides methods of assessing prognosis of a disease or condition in a subject comprising determining for said subject a disease phenocode, wherein said phenocode comprises (i) a set of disease-linked SNPs, (ii) microRNAs displaying sequence homology or complementarity to the disease-linked SNPs, and (iii) mRNAs encoded by protein-coding genes, wherein the mRNAs are targeted by the microRNAs, and wherein the disease-linked SNPs exert a regulatory effect in trans; and assessing prognosis of the disease based on said phenocode. In some embodiments, the methods of assessing prognosis of a disease or condition in a subject are performed in computer system such that a reported analysis for said phenocode is presented on a display, stored in a computer-readable medium, determined on a computer, and/or displayed on a readable device.

Another aspect of the invention includes methods of assessing risk of a developing disease or condition, or having a predisposition to develop disease or condition in an individual by assessing the status of the molecular components of a disease phenocode identified according to any of the methods disclosed herein. Further, the invention was also includes methods for the identification of therapeutic and/or preventive compounds by assessing the effect of compounds on profiles one or more molecular components of the disease phenocode identified using any of the methods of the invention and selecting those compounds that cause the reversal of molecular profiles of the disease phenocode associated with specific diseases or conditions.

The invention also describes methods of identification of phenotype-linked SNP variations and associated gene expression signatures by
1) Identifying SNPs with significant associations to the phenotype of interest;
2) Identifying target genes the expression of which is associated with phenotype-linked SNPs identified in the Step 1);
3) Building a map of regulatory SNPs/target genes using data sets defined in Step 1) and Step 2);
4) Using gene sets defined in Steps 2) and 3), to identify gene expression signature(s) discriminating samples with respect to the phenotype of interest; and/or
5) Selecting phenotype-linked SNPs by cross-referencing the gene sets comprising gene expression signatures defined in Step 4) to the map of regulatory SNPs/target genes defined in Step 3).

The invention also describes methods for identifying a consensus disease phenocode comprising a set of disease-linked SNPs, microRNAs, and microRNA-targeted mRNAs encoded by protein-coding genes. A cornerstone of this method is the idea that genetic and molecular targets relevant to disease phenotypes are defined by small non-coding RNA intermediaries displaying sequence homology/complementarity to the disease-linked SNPs/microRNAs and exerting an effect on disease target genes in trans. Such a method may involve any (or all) the following steps:
1) Identifying SNPs with significant associations to the disease of interest;
2) Identifying microRNAs with significant sequence homology/complementarity to the SNP identified in Step 1);
3) Building a sequence homology map of SNPs and microRNAs identified in Step 1) and Step 2);
4) Identifying top-scoring SNPs and microRNAs displaying the most sequence homology events;
5) Identifying mRNAs encoded by protein-coding genes which are targeted by the microRNAs defined in the Step 4); and
6) Identifying top-scoring protein-coding genes encoding mRNAs which are targeted by the largest number of microRNAs defined in Step 4).
   Top-scoring variant genomic sequences are those SNP sequences which manifest homology or complementarity to the most microRNAs at the level equal to or lower than the default level of the statistical threshold for the e-value, for example, of 10. Default levels of e-values are set to capture distinct sequence homology or complementarity of genomic sequences of interest to the relevant counterpart targets, such as microRNAs or mRNAs. Lower e-values reflect higher sequence homology or complementarity events; whereas higher e-values correspond to the lower sequence homology or complementarity between the corresponding sequences. Therefore, distinct levels of e-values are predicted to reflect distinct affinity-driven probability of interactions between homologous or complementary sequences resulting in quantitatively different effects on associated biological processes. Additionally, mRNA are identified that are homologous to microRNAs, however, microRNAs can be identified that are homologous to mRNAs.

### BRIEF DESCRIPTION OF THE DRAWINGS

FIGS. 1A-1F show sequence homology profiling of the sncRNAs that reveals both base pair complementarily and homology between piRNA and microRNA sequences. (A) Examples of the sequence complementarity between human cluster 1 piRNAs and stem-loop sequences of the hsa-mir-665 and hsa-mir-339 and sequences of mature microRNAs hsa-mir-339-5p and hsa-mir-575; (B) Examples of the sequence complementarity between human piRNAs and repeat sncRNAs and sequences of mature microRNAs the hsa-mir-518a-3p and hsa-mir-518-d-3p; (C) Genomic position of the human piRNA cluster 1 manifesting a non-random pattern of the sequence homology profile to 42 human microRNAs; (D) Top scoring 27 human microRNAs homologous to multiple piRNA transcripts encoded by the continuous −24 kb DNA sequence of the chromosome 15 comprising the human piRNA cluster 1; (E) Highly repetitive pattern of the sequential microRNA sequence homology profile generated for 895 piRNA transcripts derived from human piRNA cluster 1. Profiles are presented for 9 consecutive segments of the −24 kb region of the human chromosome 15 divided into segments generating 100 piRNA transcripts; (F) Random pattern of the microRNA sequence homology profile for the 47 sncRNA transcripts derived from repeats.

FIGS. 2A-2F show sequence homology profiling of the master trans-SNP regulatory loci that reveals allele-associated similarity to stem-loop micro-RNA sequences. (A) Example of the sequence homology between rs6852441 and stem-loop sequence of the hsa-mir-553; (B-D) Different alleles of the master trans-SNPs rs6852441 (B), rs14350 (C), rs1889229 (D) manifest distinct sequence homology profiles to human micro-RNAs; (E) Evolutionary conservation of the sequence homology profile of the master trans-SNP rs1413229 to the micro-RNA-34a; (F) Sequence homology profiling of the G/T alleles of the master trans-SNP rs210132 (BAK1 host gene) and A/G allele of the mater trans-SNP rs881878 (EGF host gene) identifies common micro-RNA homolog hsa-mir-125b targeting BAK1 host gene.

FIG. 3 shows an information-centered model of phenotype-defining functions of sncRNAs in processing, alignment, and integration of the flow of genetic information in a cell. By asserting the integrative regulatory effects on flow of genetic information in a cell, the RNP complexes of sncRNAs and Argonaute protein families (informasomes) define the unique quantitative balance of the phenotype-building blocks. The main function of informasomes is postulated to facilitate the stochastic (random and probabilistic) rather than the deterministic mode of choices in a sequence of regulatory events defining the phenotype.

FIG. 4 shows a sequence homology profiling of SNPs linked to multiple common human diseases that reveals allele-associated homology patterns to microRNAs. Notably, in all examples shown, the alleles linked to the increased risks of diseases manifest markedly higher sequence homology to the corresponding microRNAs, which is reflected by the greater values of homology scores and the lower levels of e-values. These data support the hypothesis that allele-associated differences in SNP sequence homology to microRNAs may be causally related to disease phenotypes. In all examples shown in FIG. 4, higher microRNA-targeting potency of the risk alleles is postulated. CD, Crohn's disease; CAD, coronary artery disease; RA, rheumatoid arthritis; T1 D, type 1 diabetes. Risk alleles are shown in boxes and were defined based on previously published data.

FIG. 5 shows the correlations of patterns of allele-associated changes of the sequence homology profiles of disease-associated SNPs and corresponding KPNA1-targeting microRNAs with KPNA1 mRNA expression levels in patients with Crohn's disease, bipolar disorder, and type 2 diabetes.

FIG. 6 shows sequence homology profiling of SNPs linked to multiple common human diseases reveals that allele-associated homology patterns to microRNAs. Notably, in all examples shown, the alleles linked to the increased risks of diseases manifest markedly higher sequence homology to the corresponding microRNAs which is reflected by the greater values of homology scores and the lower levels of e-values. The allele-specific e-values are shown in boxes at the top of bars. CD, Crohn's disease; CAD, coronary artery disease; RA, rheumatoid arthritis; T1D, type 1 diabetes. Risk alleles are shown in boxes and were defined based on previously published data.

FIGS. 7A-7D show correlations of patterns of allele-associated changes of the sequence homology profiles of disease-linked SNPs and corresponding KPNA1-targeting microRNAs with KPNA1 mRNAs expression levels in patients with Crohn's disease and bipolar disorder. Higher homology to KPNA1-targeting microRNAs of the multiple risk alleles in patients with Crohn's disease (FIG. 7A) is predicted to have a cumulative increased microRNA-interference effect, which would diminish cumulative KPNA1 mRNA-targeting potency of microRNAs and is associated with increased KPNA1 mRNA expression levels (FIGS. 7A, C, D). In contrast, lower SNP/microRNA sequence homology-driven decreased microRNA-interference potential of the multiple risk alleles in patients with bipolar disorder (FIG. 7B) is predicted to increase a cumulative KPNA1 mRNA-targeting potency of microRNAs and is associated with decreased KPNA1 mRNA expression levels (FIGS. 7B, C, D). Definitions of top-scoring disease-linked SNPs and corresponding microRNAs are described in the Table 6, infra. Risk alleles are underlined and were defined based on previously published data. P values were calculated using two-tailed T-test. KPNA1 mRNA targeting potency of individual microRNAs was estimated using the context scores shown in boxes at the bases of bars in FIGS. 7A and 7B. The lower values of context scores reflect the higher mRNA targeting potency of the microRNAs. The estimates of the KPNA1 mRNA-targeting potency of individual microRNAs in the allele-specific context (FIG. 7C) were derived by multiplying the corresponding context scores by the sequence homology e-values shown in boxes at the top of bars in FIGS. 7A and 7B.

FIG. 8 shows the segregation of multiple T2D risk alleles into two distinct patterns of microRNA homology profiles of disease-associated SNPs with sequence homology to the KPNA1 mRNA-targeting microRNAs. Examples of patterns of decreased (top left panels) and increased (top right panel) microRNA homology of multiple T2D risk alleles are shown. The bottom left panel shows the summary of the allele-associated changes of the microRNA homology profiles of the identified in this study top-scoring T2D-linked SNPs with homology to the KPNA1-targeting microRNAs. Negative numbers above the homology score bars are the context scores representing the relative strength of the KPNA1 mRNA-targeting potency of individual microRNAs as defined by the TargetScan algorithm. Lower values of the context scores represent higher KPNA1 mRNA-targeting potency. The bottom right panel shows the predicted KPNA1 mRNA-targeting potency in T2D patients and normal subjects for identified in this study 24 T2D-linked microRNAs with distinct allele-associated sequence homology profiles to the T2D-associated SNPs. Note that relatively small changes of the KPNA1-targeting potency driven by individual risk alleles for each microRNA result in ˜4-fold increase of the cumulative KPNA1 mRNA-targeting potency in T2D patients. Risk alleles for each SNP are shown in the left columns.

FIG. 9 shows the segregation of multiple KPNA1 mRNA-targeting microRNAs linked to the human RA phenotype into two distinct classes based on predicted changes of the KPNA1-targeting potency. It is hypothesized that changes in the KPNA1-targeting potency are driven by the risk allele-associated changes in sequence homology of the SNPs to the corresponding microRNAs. The top left panel shows the summary of allele-associated changes of the microRNA homology profiles of identified in this study multiple top-scoring RA-linked SNPs with sequence homology to the KPNA1-targeting microRNAs. Negative numbers above the homology score bars are the context scores representing the relative strength of the KPNA1 mRNA-targeting potency of individual microRNAs as defined by the TargetScan algorithm. Lower values of the context scores represent higher KPNA1 mRNA-targeting potency. The top right panel shows 10 RA-linked microRNAs with increased and 7 RA-linked microRNAs with decreased KPNA1-targeting potency in RA patients identified in this study. The bottom panels summarize the results of a similar analysis carried out for 4 RA-associated SNPs linked to the TRAF1/C5 locus and 4 RA-associated SNPs linked to the STAT4 locus. Note, that in RA patients, the predicted KPNA1-targeting potency is higher for microRNAs with sequence homology to the STA4 locus-linked SNPs, which is reflected by the lower value of the cumulative KPNA1-targeting score. In contrast, the predicted KPNA1-targeting potency is lower for microRNAs with sequence homology to the TRAF1/C5 locus-linked SNPs which is reflected by the higher value of the cumulative KPNA1-targeting score.

FIG. 10 shows the distinct KPNA1 gene expression phenotypes in RA and T2D patients. Microarray analysis reveals decreased KPNA1 mRNA expression level in peripheral blood mononuclear cells (PMBC) and synovial fluid mononuclear cells (SFMC) from RA patients (top two panels), whereas in kidneys of T2D patients with diabetic nephropathy (bottom right panel) and db/db mice with the experimental model of T2D diabetes (bottom left panel), the expression of KPNA1 mRNA is elevated.

FIGS. 11A-11D show a microarray analysis that reveals altered transcriptional balance of the principal components of inflammasome/innate immunity pathways in patients with multiple common human disorders. Examples of statistically significant misbalance manifested by the increased NLRP3/NLRP1 mRNA expression ratio in peripheral blood mononuclear cells (PBMC) of patients with Crohn's disease (A), Huntington's disease (B), and rheumatoid arthritis (C) are shown. An example of altered transcriptional balance of the principal components of nuclear import pathways demonstrates decreased KPNA1/KPNA6 mRNA expression ration in PBMC of RA patients (D).

FIGS. 12A-12E shows that chloroquine therapy reverses disease-associated transcriptional misbalances of the principal components of the nuclear import and inflammasome/innate immunity pathways. FIGS. 12A and 12B show the effect of the chloroquine therapy on NLRP1 and NLRP3 mRNAs expression (A) and NLRP3/NLRP1 expression ratio (B) in PBMC of malaria patients. FIGS. 12C and 12D show the effect of the chloroquine therapy on KPNA1 (C) and KPNA6 (D) mRNAs expression in PBMC of malaria patients. FIG. 12E shows the summary of the chloroquine therapy-induced reversal of disease-associated transcriptional pathology of the nuclear import and inflammasome/innate immunity pathways. In each panel, designations of columns illustrating the average mRNA expression values are (from left to right): Uninfected control subjects; Individuals with experimental asymptomatic infection; Malaria patients with acute untreated infection; Chloroquine-treated malaria patients.

FIG. 13 is a representative examples of allele-specific changes of SNP/microRNA sequence homology profiles of SNPs associated with human “master” disease genes NLRP1 (NALP1) and STAT4. Marked differences of SNP/microRNA sequence homology scores and e values between high-risk and low-risk alleles exist. Increased SNP/microRNA homology corresponds to higher homology scores and lower e values. It is hypothesized that decreased homology of the SNP allele to a given microRNA would reflect an intracellular context favoring enhanced microRNA activity against mRNA targets. Risk alleles are underlined.

FIGS. 14A-14H show a SNP-guided MirMaps of NLRP1- and STAT4-associated disease-linked SNPs that reveal distinct microRNA/mRNA targeting patterns for KPNA1 and KPNA6 genes. FIGS. 14A-B: SNP-guided MirMaps of predicted KPNA1 mRNA (A) and KPNA6 mRNA (B) targeting potency of microRNAs with distinct allele-associated sequence homology profiles to the NLRP1 promoter disease-linked SNPs; FIGS. 14C-D: Expression levels of the KPNA1 (C) and KPNA6 (D) mRNAs in PBMC of UC and CD patients; FIGS. 14E-F: SNP-guided MirMaps of predicted KPNA1 mRNA (E) and KPNA6 mRNA (F) targeting potency of microRNAs with distinct allele-associated sequence homology profiles to the STAT4 loci disease-linked SNPs; FIGS. 14 G-H; Expression levels of the KPNA1 (G) and KPNA6 (H) mRNAs in PBMC and SFMC of RA patients. Note that decreased microRNA targeting potency against the KPNA1 mRNA in a disease-state SNP context of the NLRP1 promoter SNPs is associated with increased expression of the KPNA1 mRNA in PBMC of UC and CD patients. Conversely, increased microRNA targeting potency against the KPNA1 mRNA in a disease-state SNP context of the STAT4 loci SNPs is associated with decreased expression of the KPNA1 mRNA in PBMC and SFMC of RA patients. No significant changes of either microRNA/mRNA targeting potency or mRNA expression levels of the KPNA6 gene were detected.

FIG. 15 shows an allele-specific changes of SNP/microRNA sequence homology profiles of rs2670660 SNP associated with NLRP1 (NALP1) promoter. Note marked differences of SNP/microRNA sequence homology scores and e values between high-risk and low-risk alleles. Increased SNP/microRNA homology corresponds to higher homology scores and lower e values. It is hypothesized that decreased homology of the SNP allele to a given microRNA would reflect an intracellular context favoring enhanced microRNA activity against mRNA targets. rs2670660 risk allele is underlined.

FIGS. 16A-16C show SNP-guided MirMaps of NLRP1 promoter-associated disease-linked SNP rs2670660 that reveal similar microRNA/mRNA targeting patterns for KPNA1 and KPNA4 genes. FIGS. 16A-B: SNP-guided MirMaps of predicted KPNA1 mRNA (A) and KPNA4 mRNA (B) targeting potency of microRNAs with distinct allele-associated sequence homology profiles to the NLRP1 promoter disease-linked SNP rs2670660; Figure C: Expression levels of the KPNA1 and KPNA6 mRNAs in PBMC of CD patients

FIGS. 17A-17H provides examples of relationships between mRNA expression levels and rs2670660 allele-dependent targeted potencies of the miR-374 and miR-130/301 microRNAs against predicted mRNA targets displaying in PBMC of CD and RA patients statistically significant expression changes. FIGS. 17A-D: Examples of relationships between mRNA expression levels (B; D) and rs2670660 allele-dependent mRNA targeting potencies of the miR-374 microRNA (A; C) against predicted mRNA targets displaying in PBMC of CD (B) and RA (D) patients statistically significant expression changes. FIGS. 17E-H: Examples of relationships between mRNA expression levels (F; H) and rs2670660 allele-dependent mRNA targeting potencies of the miR-130/301 microRNAs (E; G) against predicted mRNA targets displaying in PBMC of RA (F) and CD (H) patients statistically significant expression changes. Note that decreased mRNA targeting potency of the miR-374 is associated with increased mRNA expression levels of target genes in a disease state context. Conversely, increased mRNA targeting potency of the miR-130/301 is associated with decreased mRNA expression levels of target genes in a disease state context. G allele is the risk allele for the rs2670660 SNP.

FIGS. 18A-18H show a microarray analysis that reveals rs2670660 allele-associated gene expression signatures of the CD and RA phenotypes. FIGS. 18A-D: Direct correlations between mRNA expression levels and rs2670660 allele-dependent targeting potencies of the miR-374 and miR-130/301 microRNAs against predicted mRNA targets displaying in PBMC of CD and RA patients statistically significant expression changes compared to control subjects. Statistically significant correlations between mRNA expression levels and rs2670660 allele-dependent targeting potencies of the miR-374 (A; D) and miR-130/301 (B; C) microRNA against predicted mRNA targets displaying in PBMC of RA (A; B) and CD (C; D) patients statistically significant expression changes. Correlation analyses were carried out using either raw expression data (A-C) or expression values normalized to expression levels in control subjects (D). FIGS. 18E-H: Ten-gene signature expression profiles (E; G) and ten-gene signature score value distributions (F; H) within populations of UC and CD patients and control subjects (E; F) and populations of RA patients and control subjects (G; H).

FIGS. 19A-19D show SNP-guided MirMaps of NLRP1 promoter-associated disease-linked SNPs that reveal distinct microRNA/mRNA targeting patterns for HMGA1 and MYB genes. FIGS. 19A-B: SNP-guided MirMaps of predicted HMGA1 mRNA (A) and MYB mRNA (B) targeting potency of microRNAs with distinct allele-associated sequence homology profiles to the NLRP1 promoter disease-linked SNPs. FIGS. 19C-D: Expression levels of the HMGA1 and MYB mRNAs (C) and HMGA1/MYB mRNAs expression ratio (D) in PBMC of UC and CD patients. Note that decreased cumulative HMGA1 mRNA targeting potency by the microRNAs homologous to the NLRP1 promoter-associated disease-linked SNPs is correlated with the increased HMGA1 mRNA expression level in a disease state context. Altered transcriptional balance between mRNAs of the HMGA1 and MYB genes is reflected by the elevated HMGA1/MYB mRNA expression ratio in a disease state context (D)

FIGS. 20A-20J show SNP-guided MirMaps of NLRP1- and STAT4-associated disease-linked SNPs that reveal gene-specific patterns of microRNA/mRNA targeting for NLRP1 and NLRP3 genes and common profiles of aberrant NLRP1 and NLRP3 mRNA expression in PBMC of CD and RA patients. FIGS. 20A-B: SNP-guided MirMaps of predicted NLRP1 mRNA (A) and NLRP3 mRNA (B) targeting potency of microRNAs with distinct allele-associated sequence homology profiles to the NLRP1 promoter disease-linked SNPs. FIGS. 20C, 20D and 20I: Expression levels of the NLRP1 (C) and NLRP3 (D) mRNAs and NLRP3/NLRP1 mRNA expression ratio (I) in PBMC of CD patients. FIGS. 20E-F: SNP-guided MirMaps of predicted NLRP1 mRNA (E) and NLRP3 mRNA (F) targeting potency of microRNAs with distinct allele-associated sequence homology profiles to the STAT4 loci disease-linked SNPs. FIGS. 20G, 20H, and 20J: Expression levels of the NLRP1(G) and NLRP3 (H) mRNAs and NLRP3/NLRP1 mRNA expression ratio (J) in PBMC of RA patients. Note that increased microRNA targeting potency against the NLRP1 mRNA in a disease-state SNP context of both NLRP1 promoter and STAT4 loci SNPs is associated with decreased expression of the NLRP1 mRNA in PBMC of UC and CD patients as well as in PBMC and SFMC of RA patients. Conversely, decreased microRNA targeting potency against the NLRP3 mRNA in a disease-state SNP context of both NLRP1 promoter and STAT4 loci SNPs is associated with increased expression of the NLRP3 mRNA in PBMC of CD patients as well as in PBMC and SFMC of RA patients. Risk alleles are shown in boxes. PBMC, peripheral blood mononuclear cells; SFMC, synovial fluid mononuclear cells; CD, Crohn's disease; RA, rheumatoid arthritis; UC, ulcerative colitis.

FIGS. 21A-21K. FIG. 21A shows a SNP-guided microRNA map of Alzheimer's disease. FIG. 21B is a graph of 51-gene Alzheimer's signature score in peripheral blood mononuclear cells (PBMC) of Alzheimer's patients and control subjects. FIG. 21C is a graph of 48-gene Alzheimer's signature score in peripheral blood mononuclear cells (PBMC) of Alzheimer's patients and control subjects. FIG. 21D is a graph of 79-gene Alzheimer's signature score in peripheral blood mononuclear cells (PBMC) of Alzheimer's patients and control subjects. FIG. 21E is a graph of 20-gene Alzheimer's signature score in peripheral blood mononuclear cells (PBMC) of Alzheimer's patients and control subjects. FIG. 21F is a graph of 20-gene Alzheimer's signature in peripheral blood mononuclear cells (PBMC). FIG. 21G is a graph of eleven-gene Alzheimer's disease severity signature. FIGS. 21H(1)-(4) are graphs of HMGA2 Alzheimer's index, PHF17 Alzheimer's index, ITSN1 Alzheimer's index, and CNOT6 Alzheimer's index, respectively. FIG. 211 shows a multi-dimensional matrix of mRNA expression ratios of eleven genes in control subjects and Alzheimer's patients with different clinical manifestations of the severity of Alzheimer's disease. FIG. 21J is a graph of eleven-gene Alzheimer's disease severity index. FIG. 21K is a graph of eleven-gene Alzheimer's disease severity index.

FIGS. 22A-22I. FIG. 22A shows a SNP-guided autism MirMap. FIG. 22B is a graph of 154-gene MirMap-guided autism signature. FIG. 22C is a graph of 69-gene MirMap-guided autism signature. FIG. 22D is a scatter plot of 69-gene MirMap-guided autism signature. FIG. 22E is a graph of 69-gene MirMap autism signature. FIG. 22F is a graph of 69-gene MirMap-guided autism signature. FIG. 22G is a 6-gene MirMap-guided autism signature (zinc ion-binding proteins). FIG. 22H is a graph of ADAMTS9 mRNA targeting. FIGS. 22I(1)-(3) are graphs of 6-gene MirMap-guided autism signature (zinc ion-binding proteins).

FIGS. 23A-23L. FIG. 23A shows a prostate cancer MirMap. FIGS. 23B1-B3 are graphs of PTEN mRNA targeting. FIG. 23C is a graph of increased PTEN mRNA targeting in the context of prostate cancer-associated SNPs. FIG. 23D shows a colorectal cancer MirMap. FIG. 23E shows a breast cancer MirMap. FIG. 23F shows allele-specific SNP/microRNA sequence homology e-values and microRNA expression levels in prostate cancer. FIG. 23G shows allele-specific SNP/microRNA sequence homology e-values and microRNA expression levels in breast cancer. FIG. 23H is a graph of the direct correlation between allele-specific SNP/microRNA sequence homology e-values and microRNA expression levels in prostate and breast cancer patients. FIGS. 23I(1)-1(2) show the expression levels of mRNAs targeted by let-7 and miR-205 microRNAs in prostate tissues of control subjects and AJNT of prostate cancer patients and a scatter plot of their direct correlation. FIGS. 23J(1)-(2) show the expression levels of mRNAs targeted by let-7 and miR-205 microRNAs in breast epithelial cells from hyperplastic enlarged lobular units and normal terminal duct lobular units and a scatter plot of their direct correlation. FIG. 23K shows a 128-gene MirMap-guided prostate cancer signature. FIG. 23L shows a 128-gene MirMap-guided prostate cancer signature.

FIG. 24 shows a SNP-guided MirMap of the 8q24 gene desert harboring multiple loci associated with different human cancers.

FIGS. 25A-25B. FIG. 25A shows a haploview output of the 1.18-Mb 8q24 “desert” showing the five cancer-specific regions reported. Approximate positions of the genes POU5F1P1, c-MYC, and FAM84B are indicated. Correlations between SNPs in the region are indicated. Darker squares equate stronger correlations. FIG. 25B shows the correlations (r²) between SNPs with data in Table 16, infra. Darker shading corresponds to stronger correlations between SNPs.

FIGS. 26A-26D. FIGS. 26A-C are graphs showing the different regions on which 8q24 is located. FIG. 26D are graphs showing 8q24 amplicon identified by array CGH analysis in blood-surviving human prostate carcinoma cells and 8q24 amplicon identified by Q-PCR analysis in blood-surviving human prostate carcinoma cells.

FIGS. 27A-27B. FIG. 27A shows a genome-wide view of chromosomal positions of the 165 genes of PC3LN4/LNCapLN3 consensus class with increased transcript abundance levels. FIG. 27B shows the location of specific cancers on the chromosomes.

FIGS. 28A-28C. FIG. 28A shows a 15q25.1 locus SNP-guided MirMap of lung cancer. FIG. 28B is a graph of PTEN mRNA targeting. FIG. 28C is a graph of KRAS mRNA targeting.

FIGS. 29A-29Y. FIG. 29A shows a SNP-guided microRNA map of type 2 diabetes. FIG. 29B shows how the alleles correspond to the SNPs. FIG. 29C shows graphs of the potency of the mRNA targeting potency and mRNA targeting. FIGS. 29D and 29E are graphs displaying the targeting potency of microRNAs with sequence homology to different SNPs. FIG. 29F is a graph of tissue-specific patterns of the predicted KPNA1 mRNA-targeting potency of microRNAs with sequence homology to PPARG loci T2D-linked SNPs. FIG. 29G is graphs showing the potency of various mRNAs targeting type 2 diabetes and obesity. FIG. 29H shows graphs of mRNAs targeting within distinct allelic context of obesity- and type 2 diabetes-associated SNPs. FIG. 29I shows graphs of PPARG and FTO mRNA expression and a comparison of the same. FIG. 29J shows graphs of NOD2 and NLRP2 mRNA targeting in the context of obesity and NLRP5 and NLRP8 targeting in the context of obesity- and type 2 diabetes as well as PYCARD (ASC) mRNA expression. FIG. 29K shows graphs of KPNA1 and KPNA6 mRNA expression in the context of obese and not diabetic as well as in the context of type 2 diabetes. FIG. 29L shows the NLRP1 and NLRP3 mRNA expression cultured in vitro adipocytes. FIG. 29M shows graphs of KPNA1, KPNA4 and KPNA6 mRNA expression in peripheral blood mononuclear cells (PBMC) of patients treated with rapamycin analog CCI-779. FIG. 29N shows graphs of NLRP1, NLRP3 and NLRP3/NLRP1 mRNA expression in peripheral blood mononuclear cells (PBMC) of patients treated with rapamycin analog CCI-779. FIG. 29O shows graphs of KPNA1/KPNA6 mRNA expression ratio and KPNA1/KPNA6 mRNA expression ratio in peripheral blood mononuclear cells (PBMC) of patients treated with rapamycin analog CCI-779. FIG. 29P shows graphs of KPNA1/KPNA4 mRNA expression ratio and KPNA1/KPNA4 mRNA expression ratio in peripheral blood mononuclear cells (PBMC) of patients treated with rapamycin analog CCI-779. FIG. 29Q is a SNP-guided microRNA map (MirMap) of obesity. FIG. 29R shows graphs of MTB and HMGA1 mRNA targeting. FIG. 29S shows the cumulative MTB and HMGA1 mRNA targeting and their expression ratio. FIG. 29T shows graphs of the high and medium abundance transcripts of MMP11, PPARG, KPNA4, MAT2A, NLRP1, ST5, 1HPK3, RYBP, HMGA1, KPNA1, and TGBR1. FIG. 29U also shows graphs of the medium and low abundance transcripts but for ZNF650 (UBR3), OXR1, USP9X, VDP (USO1), OSMR, MYB, NLRP3, NOD2, PTEN, PTCH1, and MYBL1. FIGS. 29V(1)-V(2) show a scatter plot of 22 obesity-associated SNP/microRNA target transcripts. FIG. 29V(3) shows a scatter plot of 15 obesity associated FTO locus SNP/microRNA target transcripts. FIG. 29V(4) shows a scatter plot of 7 obesity-associated FTO and MC4R loci SNP/microRNA target transcripts. FIG. 29V(5) shows a scatter plot of 4-gene obesity signatures. FIG. 29W shows a graph of 23-gene obesity signature. FIG. 29X shows graphs of 23-, 15-, 7-, and 4-gene obesity signatures. FIG. 29Y shows graphs of HMGA1, MYB and HMGA1/MYB expression rations with rapamycin analog CCI-779.

FIGS. 30A-30I. FIG. 30A shows a SNP-guided microRNA map of schizophrenia. FIG. 30B is a graph of CYFIP1 mRNA targeting in schizophrenia. FIG. 30C is a graph of NIPA2 mRNA targeting in schizophrenia. FIG. 30D is a graph of GJA5 mRNA targeting in schizophrenia brain. FIG. 30E is a graph of NIPA2 mRNA targeting in schizophrenia brain. FIG. 30F is a graph of CYFIP1 mRNA targeting in schizophrenia brain. FIG. 30G is a graph of expression of genes located within the 1q21.1, 15q11.2, and 15q13.3 deletions in schizophrenia brain. FIG. 30H is a graph of 40-gene schizophrenia signature. FIG. 30I is a graph of expression profiles of the 40-gene signature in brain tissues (cortical samples corresponding to the crus I/VIIa area of the cerebellum) of schizophrenia patients and control subjects.

FIG. 31 shows a bipolar disorder MirMap.

FIG. 32 shows a coronary artery disease MirMap.

FIG. 33 shows a Crohn's disease MirMap.

FIG. 34 shows a hypertension MirMap.

FIG. 35 shows a rheumatoid arthritis MirMap.

FIG. 36 shows a type 1 diabetes MirMap.

FIG. 37 shows a type 2 diabetes MirMap.

FIG. 38 shows a type 2 diabetes super MirMap.

FIG. 39 shows a ulcerative colitis MirMap.

FIG. 40 shows a breast cancer MirMap.

FIG. 41 shows a prostate cancer MirMap.

FIG. 42 shows a systemic lupus erythematosus MirMap.

FIG. 43 shows a vitiligo and associated multiple autoimmune diseases (VIT) MirMap.

FIG. 44 shows a multiple sclerosis MirMap.

FIG. 45 shows an autoimmune thyroid disease MirMap.

FIG. 46 shows an ankylosing spondylitis MirMap.

FIG. 47 shows an autoimmune disorders MirMap.

FIG. 48 shows a chart of the development status of gene expression signatures for diagnostic, prognostic, and individualized therapy selection applications.

FIG. 49 depicts that sequence homology profiling of the master trans-SNP regulatory loci reveals marked similarity to stem-loop microRNA sequences.

FIG. 50 depicts allele-associated microRNA homology profiles of various master trans-SNPs.

FIG. 51 depicts examples of allele-associated microRNA homology profiles for disease-linked SNPs.

FIG. 52 depicts various master regulatory loci in human genome (including class I and class II SNP master trans-regulators (class I and class II MTRs)).

FIG. 53 depicts chromosomal positions of microRNA in various master trans-SNP regulatory loci.

FIG. 54 shows cross talk between master-regulators.

FIG. 55 demonstrates competition for common microRNA-binding sequence between master trans-SNP target genes.

FIG. 56 depicts 9q and 4q targeting by multiple master regulatory loci.

FIG. 57 demonstrates that the 4p16 master regulator locus targets 1p22 mater regulatory locus.

FIG. 58 shows regulatory cross-talk within the master trans SNP network. Specifically Type I, Type II, Type III, Type IV, and Type V interactions are shown.

FIG. 59 demonstrates that the network's host genes are targets of network's microRNAs.

FIG. 60 shows that 4q harbors multiple trans-regulatory SNPs and is targeted by multiple master loci. FIG. 60 also shows that the 4q master SNP locus host genes are targets of network's microRNA and that the network's host genes are targets of the 4q microRNAs. The Figure also shows that the PDFRA SNP in the 4q master regulatory locus regulates IRAK1 in Xq28 locus. Finally, this Figure also depicts the location of 4q SNP target genes.

FIG. 61 depicts T(X;4) translocation in blood-borne human prostate carcinoma cells.

FIG. 62 depicts a genomic scan of the chromosomal positions of the top 96 up-regulated genes in blood-borne human prostate carcinoma metastasis precursor cells.

FIG. 63 shows five Type 1 and seven Type 2 master trans-SNP loci.

FIG. 64 is a chart that lists the network's microRNAs with SNPs in bases 1-22 of conserved microRNA binding sites (Patrocles 1).

FIG. 65 is a chart that lists the microRNAs that are homologous to the master SNP loci.

FIG. 66 is a chart that lists the network's master trans-regulatory SNPs.

FIG. 67 is a chart that shows the network's microRNAs that are homologous to the master SNP loci.

FIGS. 68A-68C is a series of graphs showing the “Patrocles” polymorphism: SNP variations in the microRNA sequences and/or in the microRNA-targeted sequences of the mRNAs. FIG. 68A is a graph of relapse-free survival of 79 prostate cancer patients with distinct expression profiled of the 15-gene master trans-SNP host signature (high miRNA polymorphism). FIG. 68B is a graph of the survival of 91 early-stage lung cancer patients with distinct expression profiles of the 15-gene master trans-SNP host signature (high miRNA polymorphism). FIG. 68C is a graph of relapse-free survival of 286 early stage LN(−) breast cancer patients with distinct expression profiles of the 15-gene master trans-SNP host signature (high miRNA polymorphism).

FIGS. 69A-69C are a series of graphs showing the cancer treatment outcome predictor (“CTOP”) signatures comprising genes regulated by the single master trans-SNP locus (rs10061997). FIG. 69A is a graph of relapse-free survival of 79 prostate cancer patients with distinct expression profiles of the 12-gene rs10061997 signature (5q33 locus). FIG. 69B is a graph of the survival of 91 early-stage lung cancer patients with distinct expression profiles of the 8-gene rs10061997 signature 5q33 locus. FIG. 69C is a graph of the reoccurrence-free survival of 286 breast cancer patients with distinct expression profiles of the 12-gene rs10061997 signature (5q33 locus).

FIGS. 70A-70C are a series of graphs showing the CTOP signatures comprising genes regulated by the single master trans-SNP locus (re1202818). FIG. 70A is a graph of relapse-free survival of prostate cancer patients with distinct gene expression profiles of the 12-gene re1202181 (7q21) signature. FIG. 70B is a graph of the survival of 91 early early-stage lung cancer patients with distinct expression profiled of the 9-gene rs1202181 (7q21) signatures.

FIG. 70C is a graph of the survival of 295 breast cancer patients with distinct expression profiles of the 15-gene signature expression profiles of the 15-gene signature re1202181 ABCB1 (7q21).

FIGS. 71A-71C are a series of graphs that show CTOP signatures comprising genes regulated by the multiple SNPs of the ABCB1 (MDR1) master trans-SNP locus (7q21). FIG. 71A is a graph of relapse-free survival of prostate cancer patients with distinct expression profiled of the 20-gene 7q21 signature. FIG. 71B is a graph of the survival of 91 early-stage lung cancer patients with distinct expression profiles of the 27-gene 7q21 locus signature. FIG. 71C is a graph of the survival of 295 breast cancer patients with distinct expression profiles of the 15-gene 7q21 signature.

FIGS. 72A-72C are a series of graphs showing CTOP algorithm signatures comprising multiple SNP-based signatures. FIG. 72A is a graph of relapse-free survival of 79 prostate cancer patients with distinct expression profiles of the 6 SNP-based CTOP signatures. FIG. 72B is a graph of the survival of 91 early stage lung cancer patients with distinct expression profiled of the 9 SNP-based CTOP signatures. FIG. 72C is a graph of the relapse-free survival of 286 early stage LN(−) breast cancer patients with distinct expression profiles of the 5 SNP-based CTOP signatures.

FIGS. 73A-73L are graphs showing the relapse-free survival of breast cancer and prostate cancer patients. FIG. 73A is a graph of the relapse-free survival of 286 early stage LN(−) breast cancer patients with distinct expression profiles of the 49-transcript SNP-associated signature. FIG. 73B is a graph of the relapse-free survival of 286 early stage LN(−) breast cancer patients with distinct expression profiles of the 14-gene SNP-based signature. FIG. 73C is a graph of the relapse-free survival of 286 early stage LN(−) breast cancer patients with distinct expression profiles of the 26-gene SNP-associated signature. FIG. 73D is a graph of the relapse-free survival of 286 early stage LN(−) breast cancer patients with distinct expression profiles of the 35-gene “patrocles” polymorphism signature. FIG. 73E is a graph of the relapse-free survival of 286 early stage LN(−) breast cancer patients with distinct expression profiles of the 25-gene master trans-SNP host signature. FIG. 73F is a graph of the relapse-free survival of 286 early stage LN(−) breast cancer patients with distinct expression profiles of the 5-SNP based CTOP signatures. FIG. 73G is a graph of the relapse-free survival of the prostate cancer patients with distinct expression profiles of the 36-transcript SNP-associated CTOP. FIG. 73H is a graph of the relapse-free survival of 79 prostate cancer patients with distinct expression profiles of the 13-gene master trans-SNP signature. FIG. 73I is a graph of the relapse-free survival of 79 prostate cancer patients with distinct expression profiles of the 26-gene master trans-SNP signature. FIG. 73J is a graph of the relapse-free survival of 79 prostate cancer patients with distinct expression profiles of the 22-gene “patrocles” polymorphism signature. FIG. 73K is a graph of the relapse-free survival of 79 prostate cancer patients with distinct expression profiles of the 25-gene master trans-SNP host signature. FIG. 73L is a graph of the relapse-free survival of 79 prostate cancer patients with distinct expression profiles of the 5 SNP-based CTOP signatures.

FIGS. 74A-74F show graphs of the survival of lung cancer patients. FIG. 74A is a graph of the survival of 91 early stage lung cancer patients with distinct expression profiles of the 49-gene master-SNP signature. FIG. 74B is a graph of the survival of 91 early stage lung cancer patients with distinct expression profiles of the 10-gene master trans-SNP signature. FIG. 74C is a graph of the survival of 91 early stage lung cancer patients with distinct expression profiles of the 26-gene master trans-SNP signature. FIG. 74D is a graph of the survival of 91 early stage lung cancer patients with distinct expression profiles of the 35-gene “patrocles” polymorphism signature. FIG. 74E is a graph of the survival of 91 early stage lung cancer patients with distinct expression profiles of the 15-gene master trans-SNP host signature. FIG. 74F is a graph of the survival of 91 early stage lung cancer patients with distinct expression profiles of the 5 SNP based CTOP signatures.

FIGS. 75A-75F show graphs regarding lung cancer, prostate cancer and breast cancer. FIG. 75A is a graph of the survival of lung cancer patients with distinct expression profiles of the 5q31 locus signature. FIG. 75B is a graph of the relapse-free survival of prostate cancer patients with distinct expression profiles of the 5q31 locus signature. FIG. 75C is a graph of the recurrence-free survival of breast cancer patients with distinct expression profiles of the 5q31 locus signature. FIG. 75D is a graph of the survival of lung cancer patients with distinct expression profiles of the 7q21 locus signature. FIG. 75E is a graph of the relapse-free survival of prostate cancer patients with distinct expression profiles of the 7q21 locus signature. FIG. 75F is a graph of the recurrence-free survival of breast cancer patients with distinct expression profiles of the 7q21 locus signature.

FIG. 76 is a chart showing the master trans-SNP/microRNA regulatory network CTOP signatures.

### DETAILED DESCRIPTION

The following definitions will be used in the present application.

As used herein, “markers” refers to genes, RNA, DNA, mRNA, or SNPs. A “set or markers” refers to a group of markers.

As used herein, a “set” refers to at least one.

As used herein, a “set of genes” refers to a group of genes. A “set of genes” or a “set of markers” according to the invention can be identified by any method now known or later developed to assess gene, RNA, or DNA expression, including but not limited to measurements relating to the biological processes of nucleic acid amplification, transcription, RNA splicing, and translation. Thus, direct and indirect measures of gene copy number (e.g., as by fluorescence in situ hybridization or other type of quantitative hybridization measurement, or by quantitative PCR), transcript concentration (e.g., as by Northern blotting, expression array measurements or quantitative RT-PCR), and protein concentration (e.g., by quantitative 2-D gel electrophoresis, mass spectrometry, Western blotting, ELISA, or other method for determining protein concentration) are intended to be encompassed within the scope of the definition. In one embodiment, a “set of genes” or a “set of markers” refers to a group of genes or markers that are differentially expressed in a first sample as compared to a second sample. As used herein, a “set of genes” or a “set or markers” refers to at least one gene or marker, for example, 1, 2, 3, 4, 5, 10, 20, 30, 40, 50, 60, 70, 80, 90, 100, 200, 300, 400, 500, 600, 700, 800, 900, 1000 or more genes or markers.

As used herein, “differentially expressed” refers to the existence of a difference in the expression level of a nucleic acid or protein as compared between two sample classes, for example a first sample and a second sample as defined herein. Differences in the expression levels of “differentially expressed” genes preferably are statistically significant. Preferably, there is a 2-fold or more (for example, 2, 3, 4, 5, 6, 7, 8, 9, 10, 20, 30, 40, 50, 60, 70, 80, 90, 100, 200, 500, 1000-fold or more) increase or decrease in the expression levels of differentially expressed nucleic acid or protein. In one embodiment, there is at least a 5% (for example 5, 6, 7, 8, 9, 10, 15, 20, 25, 30, 35, 40, 45, 50, 55, 60, 65, 70, 75, 80, 85, 90, 95, 99, 100%) increase or decrease in the expression levels of differentially expressed nucleic acid or protein.

As used herein, “expression” refers to any one of RNA, cDNA, DNA, and/or protein expression.

“Expression values” refer to the amount or level of expression of a nucleic acid or protein according to the invention. Expression values are measured by any method known in the art and described herein. As used herein, “increased” refers to 2-fold or more (for example, 2, 3, 4, 5, 6, 7, 8, 9, 10, 20, 30, 40, 50, 60, 70, 80, 90, 100, 200, 500, 1000-fold or more) greater than. “Increased” also refers to at least 5% or more (for example 5, 6, 7, 8, 9, 10, 15, 20, 25, 30, 35, 40, 45, 50, 55, 60, 65, 70, 75, 80, 85, 90, 95, 99, 100%) greater than. As used herein, “decreased” refers to 2-fold or more (for example, 2, 3, 4, 5, 6, 7, 8, 9, 10, 20, 30, 40, 50, 60, 70, 80, 90, 100, 200, 500, 1000-fold or more) less than. “Decreased” also refers to at least 5% or more (for example 5, 6, 7, 8, 9, 10, 15, 20, 25, 30, 35, 40, 45, 50, 55, 60, 65, 70, 75, 80, 85, 90, 95, 99, 100%) less than.

As used herein, a “subset of genes” refers to at least one gene of a “set of genes” as defined herein. A subset of genes is predictive of a particular phenotype, for example, disease outcome, diagnosis of a particular disease of interest, prognosis of a particular disease of interest, recurrence, non-recurrence, invasiveness, non-invasiveness, metastatic, non-metastatic, localized, organ confined, tumor grade, Gleason score, survival prognosis, lymph node status, tumor stage, degree of differentiation, age, hormone receptor status, PSA level, histologic type, disease free survival, disease progression, remission, biochemical recurrence, metastatic recurrence, local recurrence, response to therapy, disease relapse, non-relapse, therapy failure and cure.

As used herein, “predictive” means that a set of genes or a subset of genes according to the invention, is indicative of a particular phenotype of interest (for example disease outcome, diagnosis of a particular disease of interest, prognosis of a particular disease of interest, recurrence, non-recurrence, invasiveness, non-invasiveness, metastatic, non-metastatic, localized, organ confined, tumor grade, Gleason score, survival prognosis, lymph node status, tumor stage, degree of differentiation, age, hormone receptor status, PSA level, histologic type, disease free survival, disease progression, remission, biochemical recurrence, metastatic recurrence, local recurrence, response to therapy, disease relapse, non-relapse, therapy failure and cure). A subset of genes, according to the invention that is “predictive” of a particular phenotype correlates with a particular phenotype at least 10% or more, for example 10, 11, 12, 13, 14, 15, 20, 25, 30, 35, 40, 45, 50, 51, 52, 55, 60, 65, 70, 75, 80, 85, 90, 95, 99 or 100%. As used herein, a “phenotype” refers to any detectable characteristic of an organism.

Preferably, a “phenotype” refers to disease outcome, diagnosis of a particular disease of interest, prognosis of a particular disease of interest, recurrence, non-recurrence, invasiveness, non-invasiveness, metastatic, non-metastatic, localized, organ confined, tumor grade, Gleason score, survival prognosis, lymph node status, tumor stage, degree of differentiation, age, hormone receptor status, PSA level, histologic type, disease free survival, disease progression, remission, biochemical recurrence, metastatic recurrence, local recurrence, response to therapy, disease relapse, non-relapse, therapy failure and cure.

As used herein, “diagnosis” refers to a process of determining if an individual is afflicted with a disease or ailment.

“Prognosis” refers to a prediction of the probable occurrence and/or progression of a disease or ailment, as well as the likelihood of recovery from a disease or ailment, or the likelihood of ameliorating symptoms of a disease or ailment or the likelihood of reversing the effects of a disease or ailment. “Prognosis” is determined by monitoring the response of a patient to therapy.

As used herein, preferably a “first sample” and a “second sample” differ with respect to a phenotype, as defined herein. A “first sample” refers to a sample from a normal subject or individual, or a normal cell line.

An “individual” or “subject” includes a mammal, for example, human, mouse, rat, dog, cow, pig, sheep etc. A “subject” includes both a patient and a normal individual.

As used herein, “patient” refers to a mammal who is diagnosed with a disease or ailment.

As used herein, “normal” refers to an individual who has not shown any disease or ailment symptoms or has not been diagnosed by a medical doctor.

A “second sample” refers to a sample from a patient or an unclassified individual, or an animal model for a disease of interest. A “second sample” also refers to a sample from a cell line that is a model for a disease of interest, for example a tumor cell line.

“Tumor” is to be construed broadly to refer to any and all types of solid and diffuse malignant neoplasias including but not limited to sarcomas, carcinomas, leukemias, lymphomas, etc., and includes by way of example, but not limitation, tumors found within prostate, breast, colon, lung, and ovarian tissues. A “tumor cell line” refers to a transformed cell line derived from a tumor sample. Usually, a “tumor cell line” is capable of generating a tumor upon explant into an appropriate host. A “tumor cell line” line usually retains, in vitro, properties in common with the tumor from which it is derived, including, e.g., loss of differentiation or loss of contact inhibition, and will undergo essentially unlimited cell divisions in vitro.

A “control cell line” refers to a non-transformed, usually primary culture of a normally differentiated cell type. In the practice of the invention, it is preferable to use a “control cell line” and a “tumor cell line” that are related with respect to the tissue of origin, to improve the likelihood that observed gene expression differences or differences in RNA or protein levels, are related to gene expression changes underlying the transformation from control cell to tumor.

An “unclassified sample” refers to a sample for which classification is obtained by applying the methods of the present invention. An “unclassified sample” may be one that has been classified previously using the methods of the present invention, or through the use of other molecular biological or pathohistological analyses. Alternatively, an “unclassified sample” may be one on which no classification has been carried out prior to the use of the sample for classification by the methods of the present invention.

In a preferred embodiment, the fold expression change or differential expression data are logarithmically transformed. As used herein, “logarithmically transformed” means, for example, log₁₀ transformed.

As used herein, “multivariate analysis” refers to any method of determining the incremental, statistical power of the members of a set of genes to predict a phenotype of interest. Methods of “multivariate analysis” useful according to the invention include but are not limited to multivariate Cox analysis. As used herein, “multivariate Cox analysis” refers to Cox proportional hazard survival regression analysis as performed by using the program as described in Glinsky et al., 2005, J. Clin. Investig. 115:1503.

As used herein, “survival analysis” refers to a method of verifying that a set of genes or a subset of genes according to the invention is “predictive”, as defined herein, of a particular phenotype of interest. “Survival analysis” takes the survival times of a group of subjects (usually with some kind of medical condition) and generates a survival curve, which shows how many of the members remain alive over time. Survival time is usually defined as the length of the interval between diagnosis and death, although other “start” events (such as surgery instead of diagnosis), and other “end” events (such as recurrence instead of death) are sometimes used.

Survival is often influenced by one or more factors, called “predictors” or “covariates”, which may be categorical (such as the kind of treatment a patient received) or continuous (such as the patient's age, weight, or the dosage of a drug). For simple situations involving a single factor with just two values (such as drug vs placebo), there are methods for comparing the survival curves for the two groups of subjects. For more complicated situations, a special kind of regression that allows for assessment of the effect of each predictor on the shape of the survival curve is required.

A “baseline” survival curve is the survival curve of a hypothetical “completely average” subject˜someone for whom each predictor variable is equal to the average value of that variable for the entire set of subjects in the study. This baseline survival curve does not have to have any particular formula representation; it can have any shape whatever, as long as it starts at 1.0 at time 0 and descends steadily with increasing survival time.

The baseline survival curve is then systematically “flexed” up or down by each of the predictor variables, while still keeping its general shape. The proportional hazards method (for example Cox Multivariate analysis) computes a “coefficient”, or “relative weight coefficient” for each predictor variable that indicates the direction and degree of flexing that the predictor has on the survival curve. Zero means that a variable has no effect on the curve—it is not a predictor at all; a positive variable indicates that larger values of the variable are associated with greater mortality. Knowing these coefficients, a “customized” survival curve for any particular combination of predictor values is constructed. More importantly, the method provides a measure of the sampling error associated with each predictor's coefficient. This allows for assessment of which variables' coefficients are significantly different from zero; that is: which variables are significantly related to survival.

Multivariate Cox analysis is used to generate a “relative weight coefficient”. As used herein, a “relative weight coefficient” is a value that reflects the predictive value of each gene comprising a gene set of the invention. Multivariate Cox analysis computes a “relative weight coefficient” for each predictor variable; for example, each gene of a gene set, that indicates the direction and degree of flexing that the predictor has on a survival curve. Zero means that a variable has no effect on the curve and is not a predictor at all. A positive variable indicates that larger values of the variable are associated with greater mortality. Knowing these “relative weight coefficients” a survival curve can be constructed for any combination of predictor values.

As used herein, a “correlation coefficient” means a number between −1 and 1 which measures the degree to which two variables are linearly related. If there is perfect linear relationship with positive slope between the two variables, there is a correlation coefficient of 1; if there is positive correlation, whenever one variable has a high (low) value, so does the other. If there is a perfect linear relationship with negative slope between the two variables, there is a correlation coefficient of −1; if there is negative correlation, whenever one variable has a high (low) value, the other has a low (high) value. A correlation coefficient of 0 means that there is no linear relationship between the variables.

Any one of a number of commonly used correlation coefficients may be used, including correlation coefficients generated for linear and non-linear regression lines through the data. Representative correlation coefficients include the correlation coefficient, pX;y; that ranges between −1 and +1, such as is generated by Microsoft Excel's CORREL function, the Pearson product moment correlation coefficient, r, that also ranges between −1 and +1, that reflects the extent of a linear relationship between two data sets, such as is generated by Microsoft Excel's PEARSON function, or the square of the Pearson product moment correlation coefficient, r<2>, through data points in known y's and known x's, such as is generated by Microsoft Excel's RSQ function. The r<2> value can be interpreted as the proportion of the variance in y attributable to the variance in x.

In one embodiment, a correlation coefficient, px,y; is greater than or equal to 0.8, or is greater than or equal to 0.9, or is greater than or equal to 0.95, or is greater than or equal to 0.995. One of ordinary skill can readily work out equivalent values for other types of transformations (e.g. natural log transformations) and other types of correlation coefficients either mathematically, or empirically using samples of known classification.

In a refinement of this preferred embodiment, the magnitude of the correlation coefficient can be used as a threshold for classification. The larger the magnitude of the correlation coefficient, the greater the confidence that the classification is accurate. As one of ordinary skill readily will appreciate, the appropriate threshold can be determined through the use of test data that seek to classify samples of known classification using the methods of the present invention. The threshold is adjusted so that a desired level of accuracy (e.g., greater than about 70% or greater than about 80%, or greater than about 90% or greater than about 95% or greater than about 99% accuracy is obtained). This accuracy refers to the likelihood that an assigned classification is correct. Of course, the tradeoff for the higher confidence is an increase in the fraction of samples that are unable to be classified according to the method. That is, the increase in confidence comes at the cost of a loss in sensitivity.

According to one embodiment of the invention, the expression value, or logarithmically transformed expression value for each member of a set of genes is multiplied by a “relative weight coefficient”, as defined herein and as determined by multivariate Cox analysis, to provide an “individual survival score” for each member of a set of genes.

As used herein, a “survival score” refers to the sum of the individual survival scores for each member of a set of genes of the invention.

“Survival analysis” includes but is not limited to Kaplan-Meier Survival Analysis. In one embodiment, Kaplan-Meier survival analysis is carried out using GraphPad Prism version 4.00 software (GraphPad Software) or as described in Glinsky et al., 2005. Statistical significance of the difference between the survival curves for different groups of patients is assessed using Chi square and Logrank tests.

A p-value according to the invention is less than or equal to 0.25, preferably less than or equal to 0.1 and more preferably, less than or equal to 0.075, for example, 0.075, 0.070, 0.065, 0.060, 0.055, 0.050 etc, and most preferably less than or equal to 0.05, for example, 0.05, 0.045, 0.040, 0.035, 0.020, 0.010 etc. A “p-value” as used herein refers to a p-value generated for a set of genes by multivariate Cox analysis. A “p-value” as used herein also refers to a p-value for each member of a set of genes. A “p-value” also refers to a p-value derived from Kaplan-Meier analysis, as defined herein. A “p-value” of the invention is useful for determining if a set of genes or a subset of genes of the invention is predictive of a phenotype.

A “combination of gene sets” refers to at least two gene sets according to the invention. A “combination of gene subsets” refers to at least two gene subsets according to the invention. As used herein, the term “probe” refers to a labeled oligonucleotide which forms a duplex structure with a gene in a gene set or gene subset of the invention, due to complementarity of at least one sequence in the probe with a sequence in the gene. Probes useful for the formation of a cleavage structure according to the invention are between about 17-40 nucleotides in length, preferably about 17-30 nucleotides in length and more preferably about 17-25 nucleotides in length.

As used herein, a “primer” or an “oligonucleotide primer” refers to a single stranded DNA or RNA molecule that is hybridizable to a gene in a gene set or gene subset of the invention and primes enzymatic synthesis of a second nucleic acid strand. Oligonucleotide primers useful according to the invention are between about 10 to 100 nucleotides in length, preferably about 17-50 nucleotides in length and more preferably about 17-45 nucleotides in length.

### Phenotype-Defining Functions of Multiple Non-Coding RNA Pathways

One of the surprising revelations of the initial stage of the ENCODE project was the conclusion that more than 90% of human genome is transcribed. A major component of this vast transcriptional output is represented by highly heterogeneous families of transcripts defined as short non-coding RNAs (sncRNAs) with no or limited protein-coding potentials. The sequence homolog profiling of the 2301 human sncRNAs was carried out and sequence identities were confirmed [including 943 transintrons; 235 expressed distal intergenic sequences (EDIS); and 1005 piRNAs] as well as >1000 hypothetical transcripts derived from allelic variants of human SNP sequences with strong associations to human diseases or linkages to phenotypes established in genome-wide association studies. Unexpectedly, this analysis reveals a structural feature common for 85% of analyzed sncRNA sequences and 488 human microRNAs. This structural feature common for multiple, seemingly unrelated sncRNA pathways points to a multitude of potential functional and regulatory implications involving mechanisms of gene expression regulation, control of biogenesis, stability, and bioactivity of microRNAs, sncRNA-guided macromolecular interactions, and transcriptional basis of self/non-self discrimination by immune system. The analysis implies that hundreds thousands of non-protein-coding transcripts are contributing to phenotype-defining regulatory and structural features of a cell. Therefore, definitions of genes as structural elements of a genome contributing to phenotypes should be expanded beyond the physical boundaries of mRNA-encoding units.

Thus, an information-centered model of a cell suggesting that informasomes (the RNP complexes of sncRNAs and Argonaute proteins) represent the intracellular structures that provide the increasingly complex structural framework of genomic regulatory functions in higher eukaryotes to facilitate the stochastic (i.e. random and probabilistic) rather than the deterministic mode of choices in a sequence of regulatory events defining the phenotype. Argonaute proteins are the catalytic components of the RNA-induced silencing complex (RISC), which is the protein complex responsible for the gene silencing phenomenon known as RNA interference (RNAi). Argonaute proteins bind small interfering RNA (siRNA) fragments and have endonuclease activity directed against messenger RNA (mRNA) strands that are complementary to their bound siRNA fragment. The proteins are also partially responsible for selection of the guide strand and destruction of the passenger strand of the siRNA substrate.

Common Features of sncRNAs as Non-Protein-Coding Elements of a Genome Contributing to Phenotypes

Depictions of genes as genomic regions with strict physical boundaries, which are primarily designed to generate polyadenylated protein-encoding mRNAs are rapidly evolving into a more complex and less protein-centric image of highly efficient conversion of linear genetic code into multidimensional transcriptional RNA vectors collectively contributing to quantitative features of a phenotype. This rapid evolution is captured in the definition of a gene as “ . . . a union of genomic sequences encoding a coherent set of potentially overlapping functional products,” which underscores the central role of experimental identification of the genome-driven biological function-altering events for conceptually sound segregation of phenotype-defining elements of a genetic code into structure-associated definitions of genes. (Gerstein et al., What is a gene, post-ENCODE? History and updated definition. Genome Res. 17:669-681 (2007)).

One of the most compelling experimental lines of evidence supporting this concept has emerged from recent whole-genome transcript mapping studies in which genome-scale highly efficient transcription of introns and intergenic sequences was documented and promoter-associated short RNAs (PASR) and termini-associated short RNAs (TASR) were discovered. Thus, linear genomic units previously defined as protein-coding genes appear to generate a family of transcriptional products comprising highly complex networks of interleaved, structurally diverse RNAs that are likely functionally associated to contribute to phenotypes.

Concurrently, several common features of biogenesis and structural-functional characteristics for seemingly unrelated sncRNA families have also been documented:
1. Biogenesis of many sncRNAs utilizes nuclease-mediated mechanisms of post-transcriptional processing of large precursor transcripts
2. Mechanisms of action of sncRNAs involve nucleic acid's complementarities, which drives target recognition and nuclease targeting and which does not require a perfect Watson-Crick pairing
3. Biologically active forms of sncRNAs are bound to specific proteins and represent essential structural components of specialized RNP complexes which often posses a nuclease activity
4. Expression profiles of sncRNAs manifest clearly defined tissue- and cell type-specific patterns which is consistent with their regulatory and phenotype-defining functions
5. Many sncRNAs have no or very limited protein-coding potentials

One of the logical consequences of a genome-wide pervasive transcription rule and an apparent lack of perfect Watson-Crick pairing requirement for bioactivity of sncRNAs is the prediction that transcriptional output has the capacity to generate multiple transcripts, the sequence homology potentials of which would be sufficient to affect the biogenesis, stability, and bioactivity of sncRNAs.

MicroRNA and piRNA

The most famous member of the sncRNA clan is the microRNA super-family. Expression of at least one-third of all protein-coding genes is negatively regulated by several microRNA-mediated nuclease-targeting mechanisms, most of which appear linked to the translation-associated events. Phenomenologically essential role of microRNAs is firmly established in a multitude of physiological and pathological conditions such as development, cell division and differentiation, inflammation, etc. Altered expression and function of microRNAs have been documented for a broad spectrum of human disease ranging from multiple types of cancer to heart diseases. Biogenesis of microRNAs derived from both the canonical microRNA pathway and the recently discovered mirtron microRNA pathway requires sequential processing of larger primary precursor transcripts by the consecutive cleavages by specific endonuclease enzymes.

Strictly deterministic models driven by the analogy with the siRNA mode of action would postulate a uniform mechanism of microRNA/mRNA targeting primarily mediated by the perfect Watson-Crick pairing of the seed/target sequences. However, this is clearly not the case for microRNAs. Seed/target pairing is necessary, but not sufficient, to reliably predict the in vivo mRNA targets for microRNAs. A recent breakthrough in the understanding of the structural determinants of specificity of microRNA-mediated mRNA targeting beyond the seed pairing explains why mRNAs with indistinguishable primary sequence-defined seed target potential have markedly distinct response to microRNA targeting in vivo. Most significantly, protein expression-based assays demonstrate that mRNAs identified as potential microRNA targets based on seed pairing uniformly failed to respond to microRNA challenge in vivo when target regions reside within unfavorable sequence context defined by the target prediction algorithm. Interestingly, at least some mRNAs with identical favorable context scores demonstrate markedly different response to the microRNA challenge in vivo.

Several experimental observations seem to expose apparent logical gaps in current theoretical models of microRNA biogenesis and functions. It not know why mRNAs of some genes appear targeted by only a few microRNAs. However, many mRNAs are potential targets for dozens or even several hundreds of microRNAs. This conclusion remains correct regardless of which microRNA target prediction algorithm is used to define the microRNA/mRNA targeting. Likewise, it is not completely understood why microRNAs with indistinguishable primary sequence-defined seed target potential may have markedly distinct mRNA targeting activities in vivo. Finally, except for highly abundant mRNAs, most microRNAs and potential mRNA targets are co-expressed in the same cells and tissues, which is in apparent contradiction with the postulated gene expression inhibitory function of microRNAs mediated by targeting of corresponding mRNA for degradation.

Mature sncRNA species generated in canonical small interfering RNAs (siRNAs) and microRNAs pathways are derived from double-stranded RNA precursors by the Dicer endonuclease-mediated cleavage. siRNAs and microRNAs function in complexes with Argonaute-family proteins to silence translation or to destroy mRNA targets. However, the most diverse class of sncRNAs is a product of alternative biogenesis pathways, which does not require the cleavage of double-stranded RNA precursors. Recently, a distinct class of 24- to 30-nucleotide-long RNAs was discovered which are produced by a Dicer-independent mechanism and associates with Piwi-class Argonaute proteins. Small RNA partners of Piwi proteins were termed Piwi-interacting RNAs (piRNAs). Piwi proteins and piRNAs form a regulatory system distinct from the canonical siRNA and miRNA pathways. piRNA populations are extremely complex, with recent estimates placing the number of distinct mammalian pachytene piRNAs at >500,000.

Similar to microRNAs, piRNAs guide Argonaute protein complexes to trigger the target silencing through a complementary base-pairing. Silencing is achieved by target destruction via co-recruitment of accessory factors or through the endonucleolytic activity of Argonaute protein itself. Like microRNAs, piRNAs carry a 5′ monophosphate group and exhibit a preference for a 5′ uridine residue.

Several lines of experimental evidence support a model of piRNA biogenesis in which a single transcript traverses an entire piRNA cluster and is subsequently processed by endonuclease cleavage into mature piRNAs. The sequential combinations of the piRNA biogenesis steps generating mature piRNAs associated with Argonaute proteins and complementary base pairing-guided target cleavage steps mediated by the piRNA/Argonaute complexes can form a feed-forward self-amplifying loop. It is important to note that comparisons at high stringency of D. melanogaster piRNAs to transposons present in related Drosophilids show a lack of perfect complementarity. However, when even a few mismatches are permitted, it seems evident that piRNA loci might have a potential to protect against horizontal transmission of these heterologous transposable elements. Thus, similar to the bioactivity of microRNAs, piRNA-guided endonuclease-mediated degradation of target sequences also does not require a perfect Watson-Crick base pairing.

The existence of a feed-forward amplification loop has been related to clonal expansion of immune cells with the appropriate specificity following antigen stimulation, leading to a robust and adaptable response. Thus, similar to the adaptive immune response, this piRNA-guided transposon silencing pathway has both genetic and adaptive components leading to self-amplification of complementary RNA sequences.

Taken together, these observations indicate that important details of biogenesis, stability, and bioactivity of sncRNAs, in particular, biologically significant regulatory mechanisms of editing microRNA activity in vivo, remain unknown. Here, the sequence homolog profiling of the 2301 human small non-coding RNA transcripts was carried out with confirmed sequence identities [including 943 transintrons; 235 expressed distal intergenic sequences (EDIS); and 1005 piRNAs; 47 sncRNAs derived from repeats; 71 sncRNA transcripts, including 12 PASRs and 34 TASRs, expression of which was identified by microarray analysis and validated using independent analytical methods such as Northern and/or quantitative RT-PCR] as well as >1000 hypothetical transcripts derived from allelic variants of human SNP sequences with strong associations to human diseases or linkages to phenotypes established in genome-wide association studies. Unexpectedly, this analysis reveals a structural feature common for ˜85% of analyzed sncRNA sequences and 488 human microRNAs. Based on these findings, an information-centered model of a cell postulates that informasomes (the RNP complexes of sncRNAs and Argonaute proteins) constitute the intracellular structures which provide the increasingly complex regulatory functions in higher eukaryotes to facilitate the stochastic (random and probabilistic) rather than deterministic mode of regulatory choices in a sequence of events defining the phenotypes.

piRNAs, Transposon Silencing, and MicroRNA Biogenesis and Stability

Post-transcriptional control of mRNA abundance levels is an important component of global gene expression regulation in mammalian cells. It has been suggested that extreme diversity of pachytene piRNAs may allow MIWI and MILI complexes to exert broad effects on the transcriptome through miRNA-like mechanism. Consistent with this idea, the loss of Miwi protein has been linked to changes in the abundance levels of several developmentally-important mRNAs.

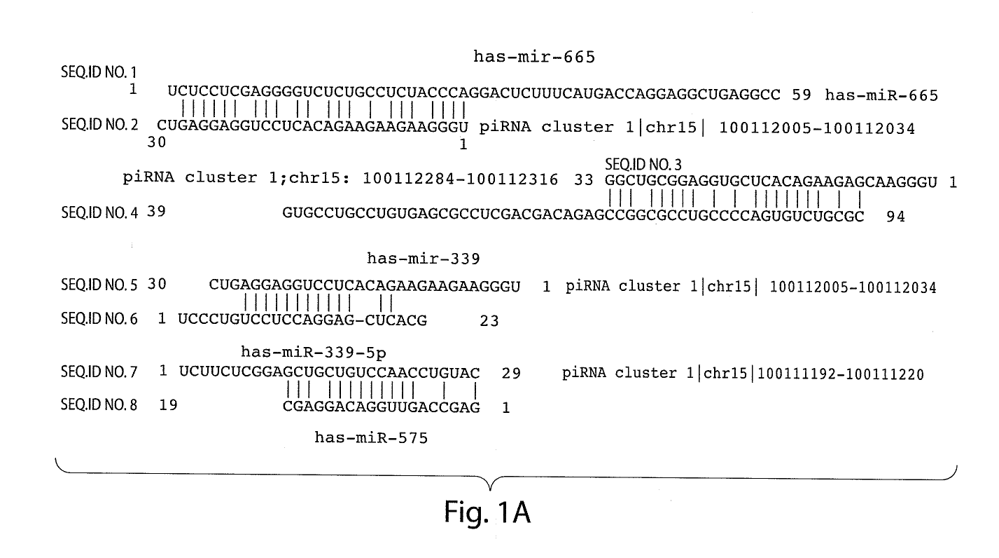

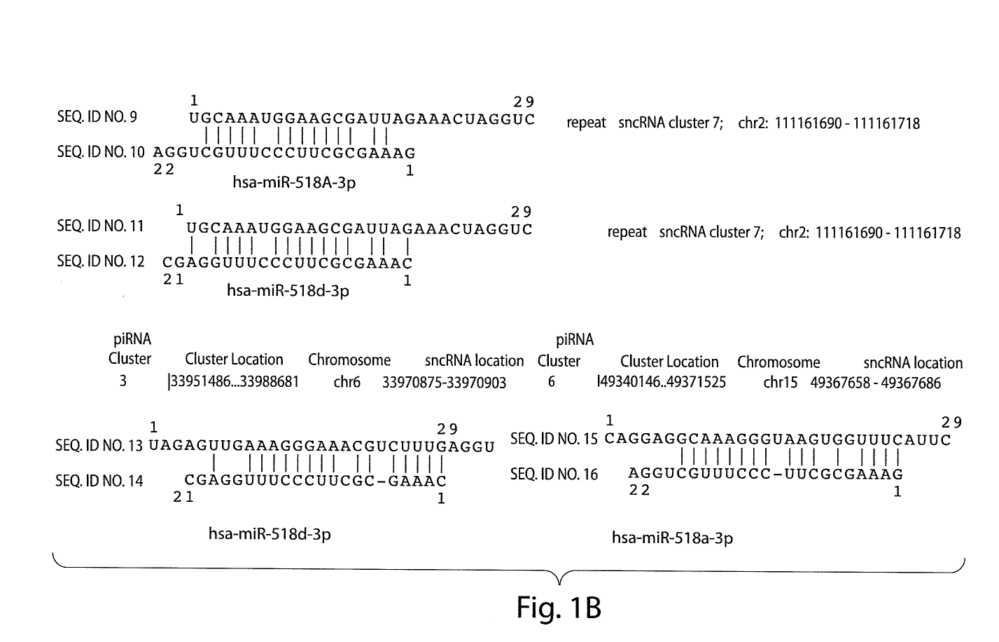

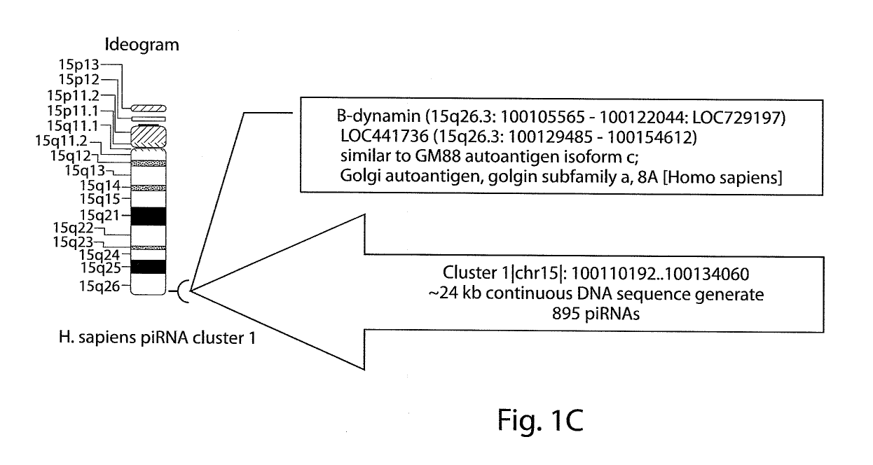

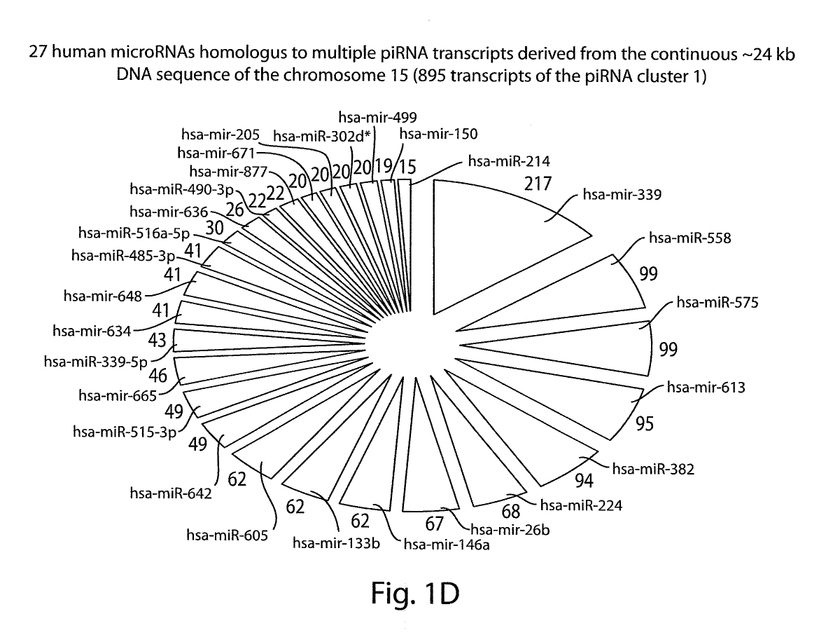

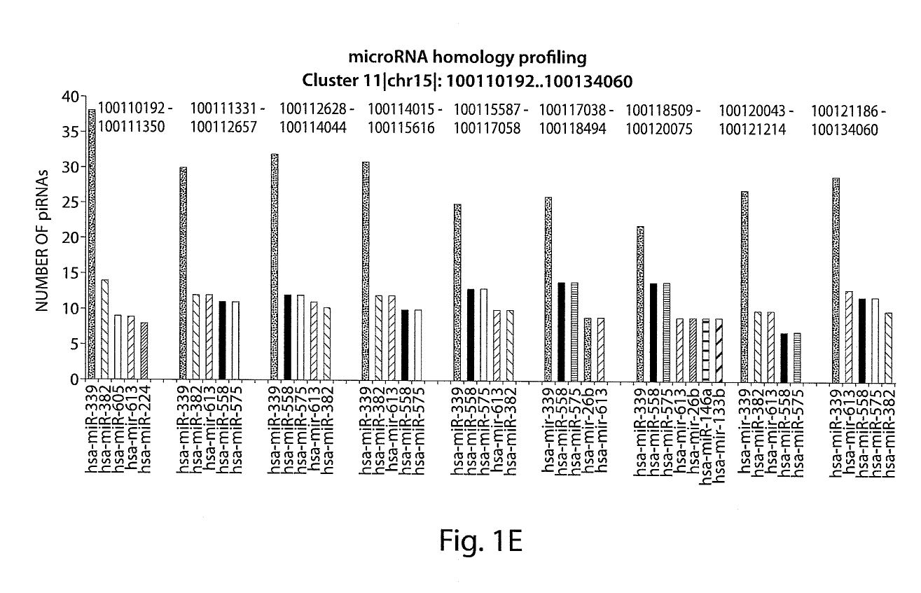

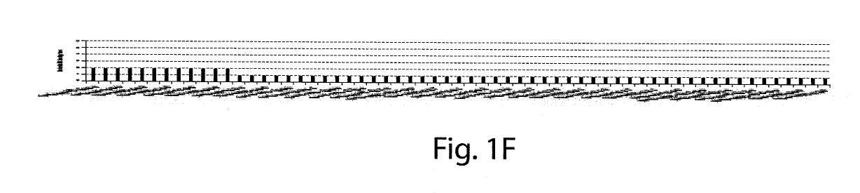

It is highly likely that potential multiple regulatory effects of distinct classes of sncRNAs on gene expression involve a base complementarity-driven guiding mechanism mediating the specificity of regulatory interactions. One of the intriguing regulatory concepts exploiting this idea may be focused on sequence complementarity of multiple sncRNA classes to the microRNAs. To explore the validity of this hypothesis, the sequence homology profiling of the human piRNAs and microRNAs was carried out. As shown in FIG. 1, this analysis reveals examples of apparent systematic sequence complementarity and homology between microRNAs and different sncRNAs, including piRNAs and repeat sncRNAs. These results suggest that microRNAs, similar to piRNAs, may contribute to the transposon silencing and may trigger the function of a feed-forward amplification loop which may initiate a generation cycle of the primary sets of piRNAs in a cell. Alternatively, piRNAs and piRNA-containing RNP complexes may influence the biogenesis, stability, and bioactivity of microRNAs via base complementarity-guiding mechanisms. Consistent with this idea, sequence homology profiling demonstrates non-random patterns of sequence homology interactions between microRNAs and 895 piRNAs derived from human piRNA cluster 1 (FIG. 1). In contrast, repeat sncRNAs manifest a random pattern of sequence homology to the human microRNAs (FIG. 1). Overall, analysis of 1005 human piRNAs derived from 14 clusters residing on 9 chromosomes identifies 570 sequence homology interactions manifesting sequence homology to the 191human microRNAs (SSEARCH algorithm; E value cutoff: 10)

Transintrons: Transcribed Intronic Sequences Displaying Marked Homology to the Stem-Loop Sequences of Hundreds MicroRNA Genes

Human genome tiling array experiments identify thousands of transcribed sense/antisense sequences not detected by other methods, including more than 3,500 transcriptionally active intronic regions. The biological functions of transcribed intronic sequences remain unknown. A sequence homology profiling of the 314 intronic transcripts encoded by the DNA sequences located in regions distal from previously annotated genes (at least 10 kb) was carried out. These transcribed intronic sequences (transintrons) are derived from 149 antisense, 137 sense, and 28 sense/antisense transcriptionally active introns. Analysis identifies 113 statistically significant sequence homology interactions between sense/antisense transintrons and stem-loop microRNA sequences (search method: Wublastn; sequence database: Hairpin; E value cutoff: 10); 468 sequence homology interactions between sense transintrons and stem-loop microRNA sequences; 509 sequence homology interactions between antisense transintrons and stem-loop microRNA sequences. Overall, 89% of sense/antisense transintrons, 87% of sense transitrons, and 91% of antisense transintrons manifest significant homology to the stem-loop sequences of 70, 178, and 208 microRNAs, respectively. Collectively, 280 transintrons, many of which have statistically significant homology defined by BLAST analysis to sequences in the mouse genome, are highly homologous to the stem-loop sequences of 286 microRNAs. Most of transintrons manifest marked SNP variations and many transintron-linked SNPs display allele-associated sequence homology profiles to the stem-loop and/or mature microRNAs (SSEARCH algorithm; E value cutoff: 10). A general significance of these findings was validated by analysis of additional set of 629 transintrons identified for the −1% of the human genome in the ENCODE regions. These data suggest a possible biological function for transintrons acting as exon guardians to protect the flow of genetic information by interfering with the microRNA/mRNA interactions and/or affecting the biogenesis of the microRNAs.

Little is known regarding the biogenesis of transintrons. However, some helpful analogies may be derived from the mechanisms of biogenesis of the recently identified class of mirtron-derived microRNAs. Mirtron hairpins are defined by the action of the splicing machinery and lariat-debranching enzyme, which yield pre-miRNA-like hairpins, suggesting a role for the lariat-debranching enzymes in the generation of transintrons and implying that similar mechanisms are likely to govern initial stages of the biogenesis of transintrons.

Promoter-Associated Short RNAs (PASR) and Termini-Associated Short RNAs (TASR): Transcriptional Siblings of Protein-Coding mRNAs

Perhaps, one of the most compelling experimental illustrations supporting the concept of non-linear phenotype-defining units of a genome emerged from a recent whole-genome transcript mapping study in which promoter-associated short RNAs (PASR) and termini-associated short RNAs (TASR) were discovered. (See Kapranov P., et al., RNA maps reveal new RNA classes and a possible function for pervasive transcription, Science. 316:1484-1488 (2007)). It appears that during transcription of protein-coding genes a multitude of sncRNA species is generated, which includes sncRNAs structurally defined as PASR and TASR transcripts. Sequences of PASR and TASR transcripts often mark boundaries of the protein-coding genes, and expression of PASRs and TASRs appears to correlate with the expression state of corresponding protein-coding genes. Most recent experimental evidence supports the idea that low-copy promoter-associated RNAs are required for RNA-directed transcriptional gene silencing by guiding the epigenetic silencing complexes to the promoters of corresponding target genes. (See Kapranov P., et al., RNA maps reveal new RNA classes and a possible function for pervasive transcription, Science. 316:1484-1488 (2007)). Consistently, an appreciable fraction of protein-coding genes have expression only in the first exon and intron and the ends of almost half of human protein-coding genes are bracketed by PASRs and TASRs. However, for ˜80% of silent genes (defined as genes with <10% exons detected), no PASRs were detected by microarray analysis, suggesting that corresponding PASRs may be retained by the RNP complexes. A sequence homology profiling of 71 sncRNA transcripts, including 12 PASRs and 34 TASRs, was carried out and the expression of which was validated using independent analytical methods such as Northern and/or quantitative RTPCR. Analysis reveals that 31 of 34 (91%) TASRs, 10 of 12 (83%) PASRs, 12 of 12 (100%) sncRNAs of syntenic human-mouse regions, and 20 of 23 (87%) of sncRNAs derived from intergenic/intronic/exonic sequences manifest significant sequence homology to 125 human microRNAs (SSEARCH algorithm; E value cutoff: 10). These data suggest that base complementarity-guided interactions between sncRNAs and microRNAs constitutes an important component of gene expression regulatory network in a cell. Similar to siRNAs, microRNAs may assist in guiding the epigenetic silencing complexes to targeted promoters. Alternatively, retention of PASRs by the microRNA/Argonaute complexes may interfere with PASRmediated transcriptional gene silencing, suggesting that in certain circumstances microRNAs may elicit a stimulatory effect on gene expression. Indeed, the stimulatory effect of microRNAs on gene expression has been demonstrated experimentally. (See Vasudevan S., et al., Switching from repression to activation: microRNAs can up-regulate translation, Science. 318:1931-1934 (2007)).

Exon Guardian Functions of EDIS Transcripts: Sequence Homology Profiling Identifies Expressed Distant Intergenic Sequences (EDIS) with Marked Homology to Sequences of Hundreds MicroRNA Genes

Recent releases of the ENCODE project identify thousands of RNA molecules in human cells derived from transcriptionally active regions (TAR) of human genomes, which do not contain either previously annotated genes or detectable classical ORF sequences. Biological functions of this novel class of non-coding RNA molecules remain unknown. A sequence homology profiling of 235 intergenic transcripts was carried out and identified for the ˜1% of the human genome in the ENCODE regions. DNA sequences encoding these intregenic transcripts are located in regions distal from previously annotated genes (at least 5 kb). Analysis reveals 416 statistically significant sequence homology interactions between 163 expressed distal intergenic sequences (EDIS) and 208 stem-loop microRNA sequences (for sequences>100 bp: search method: Wublastn algorithm; E value cutoff: 10; for sequences= or <100 bp: search method: SSEARCH algorithm; E value cutoff: 10; sequence database: Hairpin. 125 EDIS transcripts manifesting 212 significant sequence homology interactions with the mature microRNA sequences were identified (sequence database: Mature;). Overall, this demonstrates that 200 of 235 (85%) of EDIS transcripts manifest 628 statistically significant sequence homology interactions with either stem-loop or mature sequences of 278 microRNAs. Importantly, sequences of many of EDIS transcripts appear evolutionary conserved and have statistically significant homology defined by BLAST analysis to sequences in the mouse genome. Sequence homology profiling reveals that most of EDIS transcripts manifest marked SNP variations and many EDIS-linked SNPs display allele-associated sequence homology profiles to the stem-loop and/or mature microRNAs (SSEARCH algorithm; E value cutoff: 10. As with transintrons, these data suggest an important biological function for EDIS transcripts acting as exon guardians to protect the flow and phenotypic expression of genetic information by interfering with the microRNA/mRNA interactions and/or affecting the biogenesis of the microRNAs.

Preliminary Evidence for a Genome-Scale Intra-Nuclear Exon Guardian Regulatory Mechanism at the Drosha/DGCR8 Stage of the miRNA Biogenesis Revealed by the Sequence Homology Profiling of the Human Trans-SNP Master Regulatory Loci

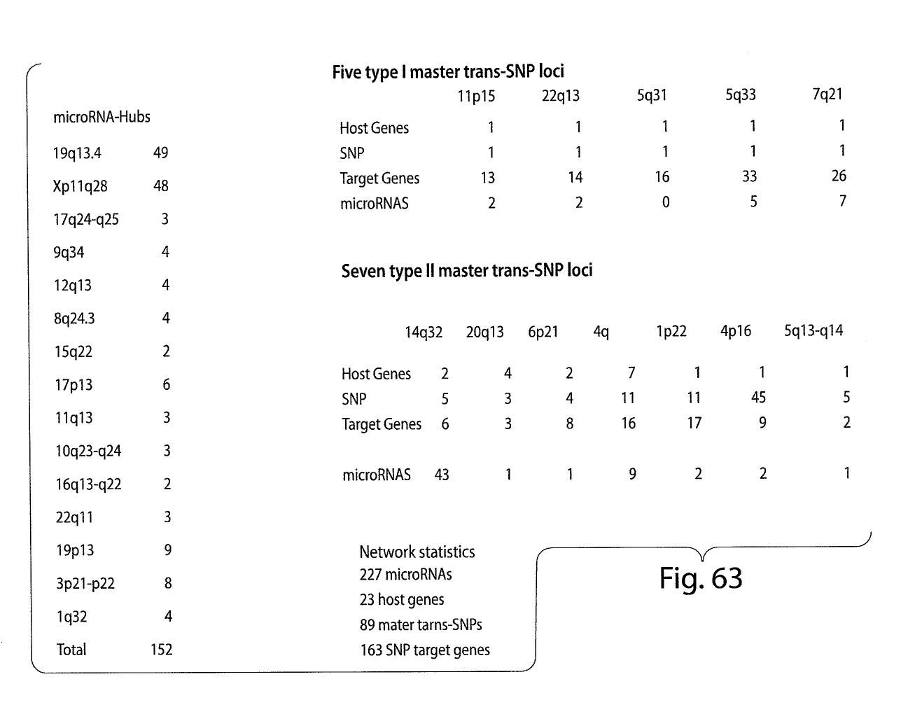

Database releases of the HapMap and ENCODE projects revolutionize the ability to understand a complex architecture of structural and functional elements of human genomes. For example, a novel class of human master regulatory SNPs manifesting statistically robust effect on expression of multiple target genes in trans were recently discovered. Despite a consensus view that this discovery holds a significant promise of unraveling the genetic basis of individual and ethnic diversities of H. sapiens, the molecular nature and precise mechanisms of these important regulatory interactions remain unknown. A database was built using the 89 master trans-SNP regulatory loci located at 12 distinct chromosomal regions of human genome (11p15; 22q13; 5q31; 5q33; 7q21; 14832; 20q13; 6p21; 4q11-q35; 4p16; 1p22; 5q13-q14) (See, FIG. 63). These master trans-SNP regulatory loci affect expression of 163 target genes in trans. Sequence homology profiling of the master trans-SNP regulatory loci using hairpin microRNA database revealed systematic marked homology between master trans-SNP sequences and 157 stem-loop miRNA sequences. This analysis identified 219 sequence homology interactions with the homology score>90.0; 126 sequence homology interactions with the homology score at least 95.0; 56 sequence homology interactions with the homology score>99.0 and E values<5.0 (SSEARCH algorithm). Many of these interactions manifest allele-specific sequence homology profiles, thereby suggesting a potential intra-nuclear regulatory mechanism at pri/pre-microRNA stages of the miRNA biogenesis. This regulatory step involves the Watson-Crick complementarity-based binding of the SNP-derived non-coding RNAs to the pri/pre-miRNAs and interference with the miRNA biogenesis at the Drosha/DGCR8 nuclear complex/nuclear export stages.

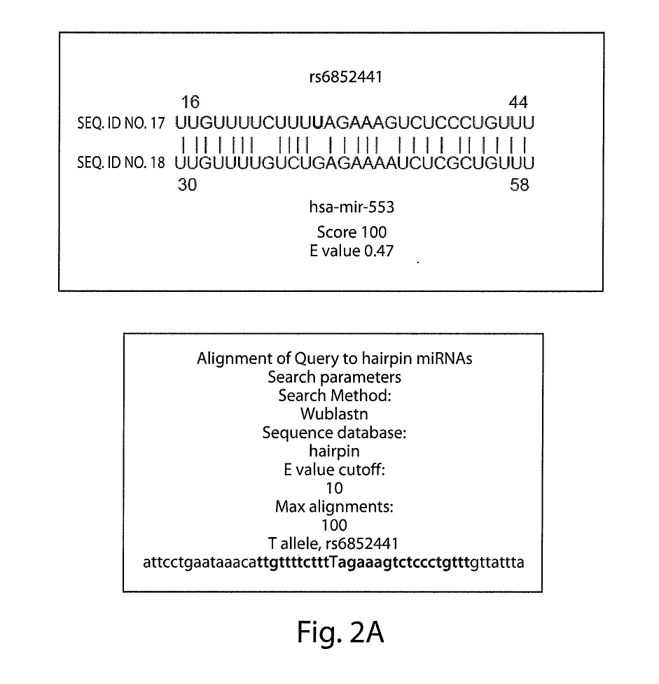

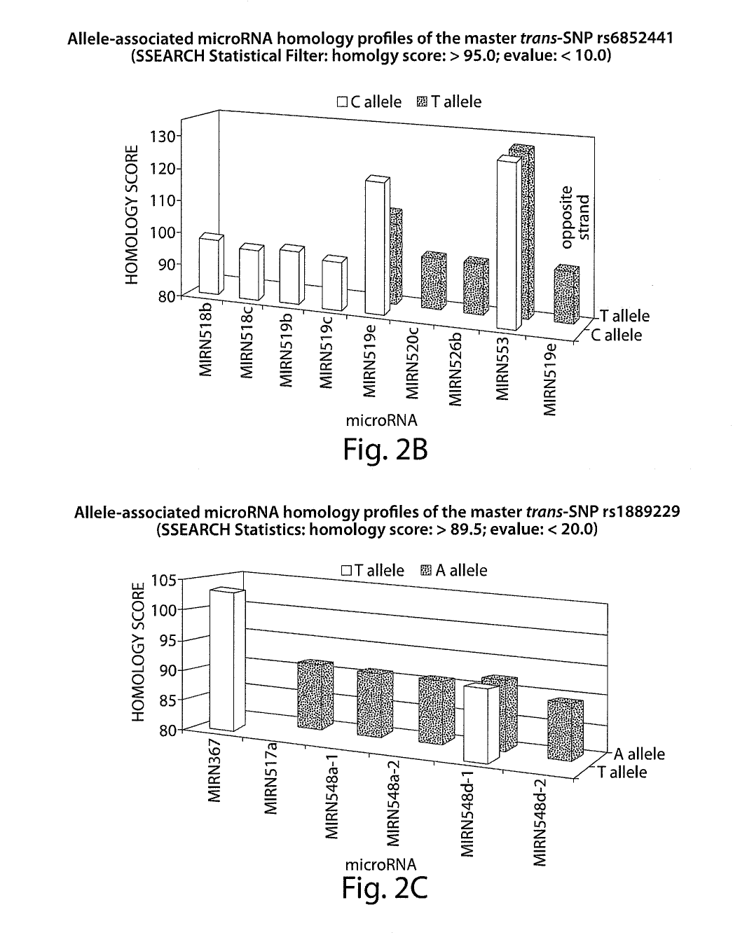

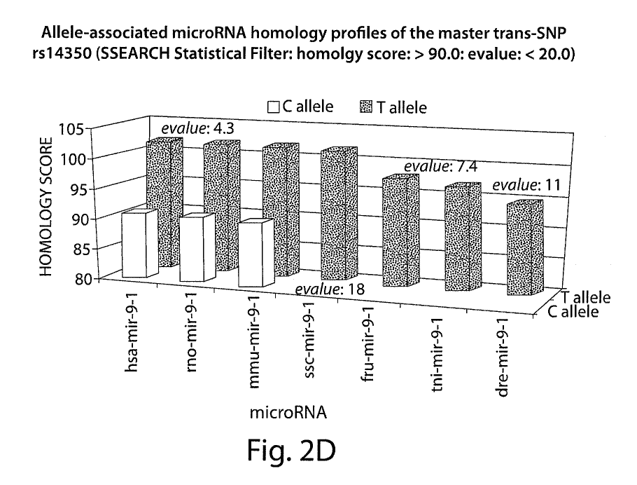

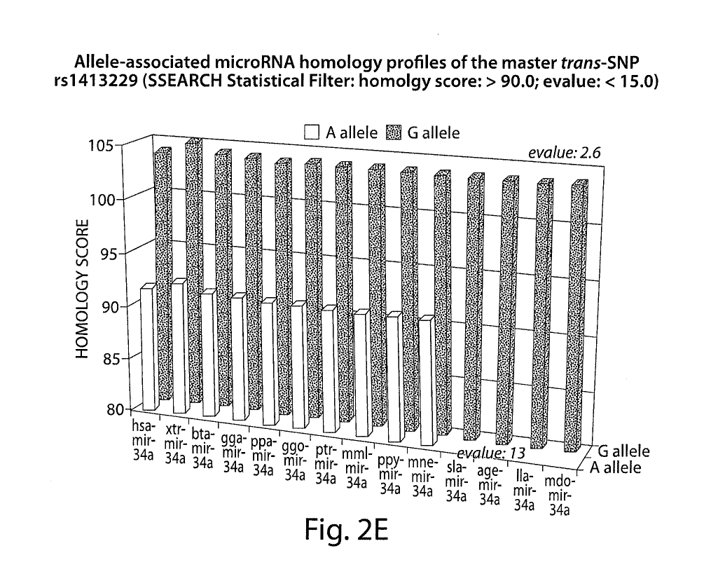

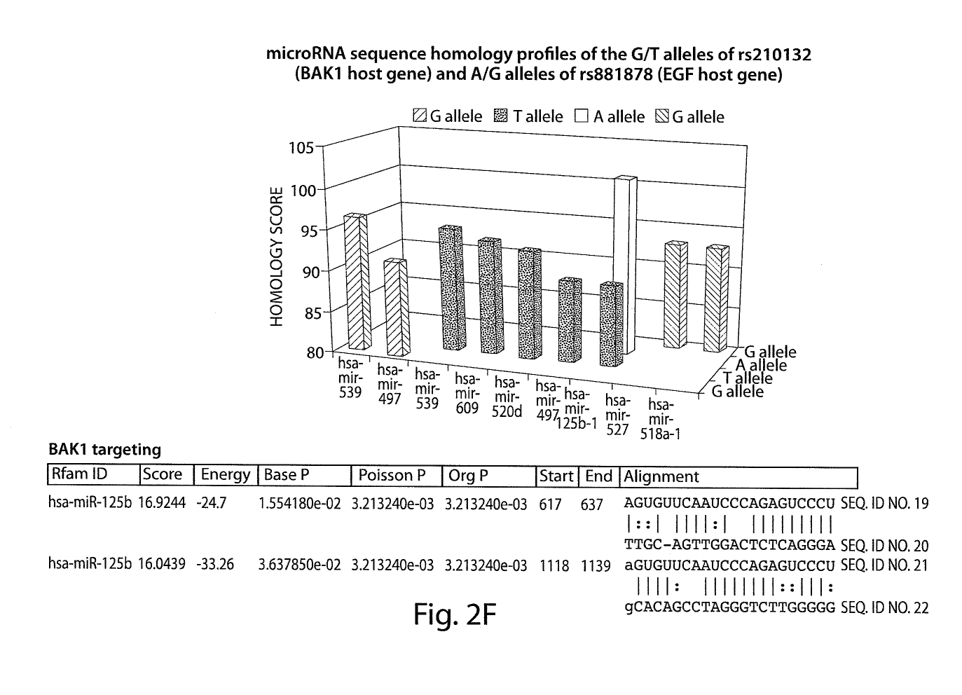

Six sequence homology interactions were identified between intronic master trans-SNP and microRNAs targeting the corresponding SNP host genes, suggesting a coordinated regulatory intron/exon cross-talk mediated by the intra-nuclear RNA/RNA interactions (FIG. 2). Many sequence homology interactions are between intronic master trans-SNP and microRNAs targeting heterologous trans-SNP host genes (196 events) or homo- and heterologus trans-SNP target genes. These data suggest the previously unrecognized biological function of the intronic sequences as the exon guardians protecting corresponding mRNAs by interfering with the biogenesis of the mRNA-targeting microRNAs.

Further analysis revealed several instances of striking evolutionary conservation of the master trans-SNP/microRNA sequence homology interactions extending across as many as 11 and 13 species, which suggests a common evolutionary origin of the trans-SNP master regulatory loci and microRNAs. Mature microRNA database searches revealed less profound sequence homology interactions between master trans-SNPs and microRNAs. Interestingly, when such interactions were detected for a given master trans-SNP locus, microRNA sequence homology profiles derived from mature and stem-loop sequences manifest both overlapping and distinct allele-specific features. A growing body of evidence supports the significance of the interactions between microRNA and their targets and SNP variations in microRNA binding sites on targeted mRNAs in heritability of complex quantitative traits. (See, Wong K K, et al., A comprehensive analysis of common copy-number variations in the human genome. Am. J. Hum. Genet. 80:91-104 (2007)). Most of the master trans-SNP homologous microRNAs identified manifest a Patrocles polymorphism (polymorphic miRNA-target interactions), thus adding a novel level of regulation to a remarkable complexity of epistatic, regulatory interactions of SNP polymorphisms and microRNAs in the heritability of the complex genetic traits in human. Consistent with this concept, 75 of 89 master trans-regulatory SNPs are targets of large-scale segmental copy number variations (CNV) in the human genome.

Trans-SNP/microRNA Master Regulatory Network

Genome-scale integration of the HapMap-based SNP pattern analysis and gene expression profiling reveals a novel class of master regulatory SNPs in human genomes manifesting statistically robust effect on expression of multiple target genes in trans. There is a broad consensus regarding the major significance of these regulatory interactions for understanding of the genetic underpinnings of population-based and inter-individual physiological and pathological diversities of H. sapiens. (See Huang R. S., et al., Identification of genetic variants contributing to cisplantin-induced cytotoxicity by use of a genome wide approach. Am. J. Hum. Genet., 81:427-437 (2007); Huang R. S., et al., A genome-wide approach to identify genetic variants that contribute to etoposide-induced cytotoxicity. Proc. Natl. Acad. Sci. USA. 104:9758-9763 (2007); Spielman R. S., et al., Common genetic variants account for differences in gene expression among ethnic groups. Nat. Genet. 39:226-231 (2007); Morley M. et al., Genetic analysis of genome-wide variation in human gene expression. Nature. 430:743-747 (2004); Cheung V. G., et al., Mapping determinants of human gene expression by regional and genome-wide association. Nature. 437:1365-1369 (2005); and Kristensen V. N., et al., Genetic variation in putative regulatory loci controlling gene expression in breast cancer. Proc. Natl. Acad. Sci. USA. 103:7735-7740 (2006)).

A master trans-SNP/microRNA network hypothesis postulates that the regulatory effect of master trans-SNP on gene expression is mediated by non-coding RNA intermediaries interacting with microRNAs. It predicts that genetic loci harboring master trans-SNP regulatory sequences are transcriptionally active and should exist as detectable transcripts. Microarray-guided genomic scans of expression of host and target genes, microRNAs, and SNPs of the master trans-SNP regulators (MTSRs) located at 12 distinct chromosomal regions of human genome were carried out. Analysis identified 5 type I MTSRs located at 11p15; 22q13; 5q31; 5q33; 7q21; and 7 type II MTSRs residing at 14q32; 20q13; 6p21; 4q11-q35; 4p16; 1p22; 5q13-q14. (See, FIG. 63). Host genes of the type I MTSRs harbor a single regulatory SNP affecting expression of multiple target genes in trans. Type II MTSRs harbor two or more (often, multiple) regulatory SNPs located in the same genomic region (often, within the boundaries of the same host gene) affecting expression of multiple target genes in trans. Chromosomal locations of the host genes of 11 of the 12 MTSRs are in close proximity to at least one (3 MTSRs), two (4 MTSRs), 5 (5q33 MTSR), 7 (7q21 MTSR), 9 (4p16 MTSR), and 43 (14q32 MTSR) of the microRNA-encoding genes. These data suggest that the trans-regulatory effects of MTSRs may be mediated by microRNAs.

Consistent with this idea, it was determined that mRNAs of essentially all MTSR host and target genes share sequences with target potentials for common sets of microRNAs. Furthermore, subsets of MTSR target genes, the expression of which are affected by multiple distinct MTSRs, are often located in the same chromosomal regions. Twelve of these chromosomal regions harboring common genetic targets of multiple MTSRs are located in close proximity to at least 2 (16q13-q22; 15q22); 3 (22q11; 10q23-q24; 11q13; 17q24-q25); 4 (1q32; 8q24.3; 12q13; 9q34); 6 (17p13); 8 (3p21-p22); 9 (19p13); 48 (Xpl 1-q28); and 49 (19q13) of the microRNA-encoding genes. These chromosomal regions are defined as microRNA “hubs”. Finally, chromosomal coordinates of subsets of MTSR targets genes are in close proximity to MTSR host genes residing on distinct chromosomes. Notably, most of the master trans-regulatory SNPs are located within introns of host genes.

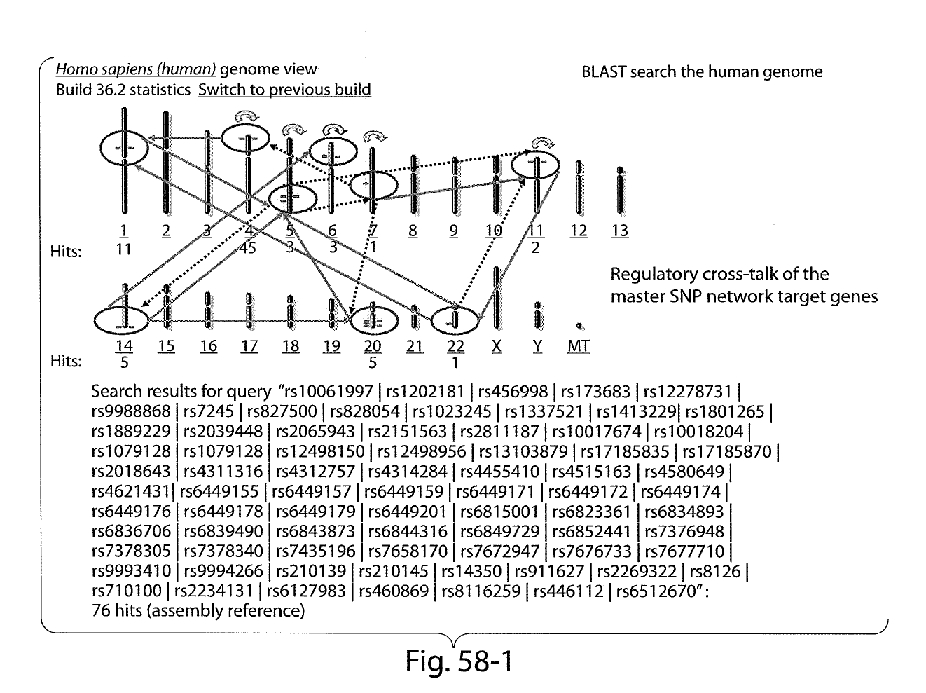

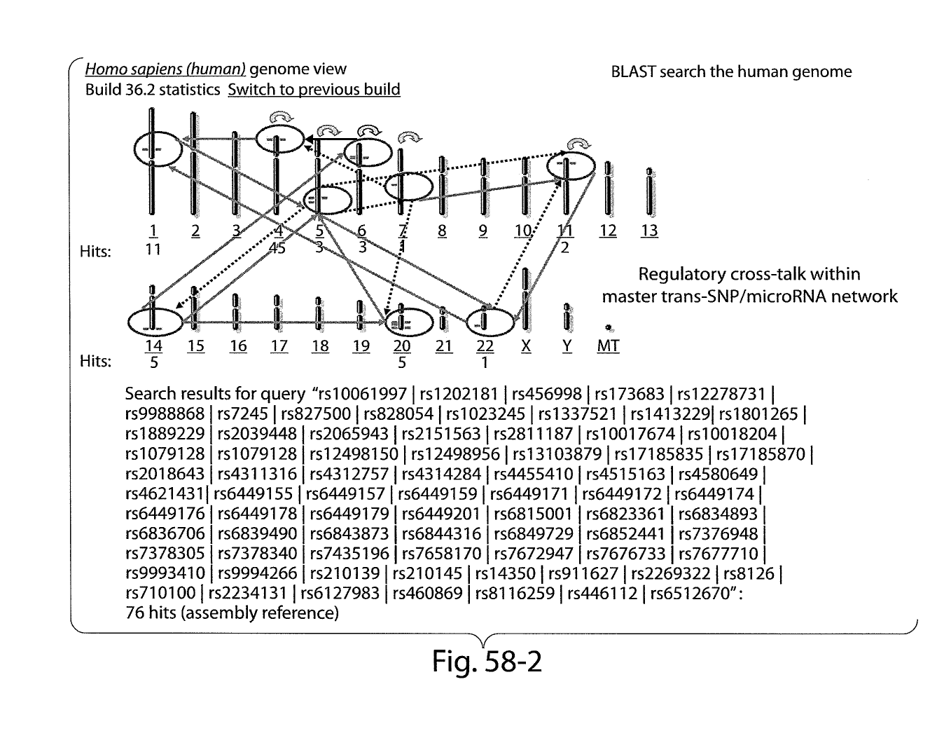

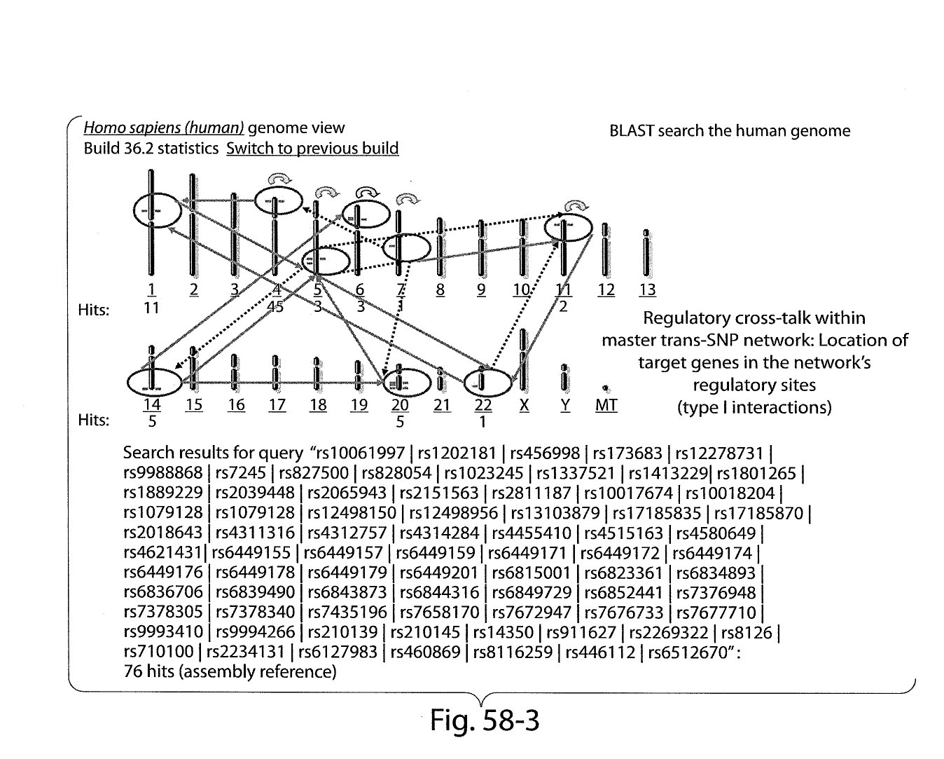

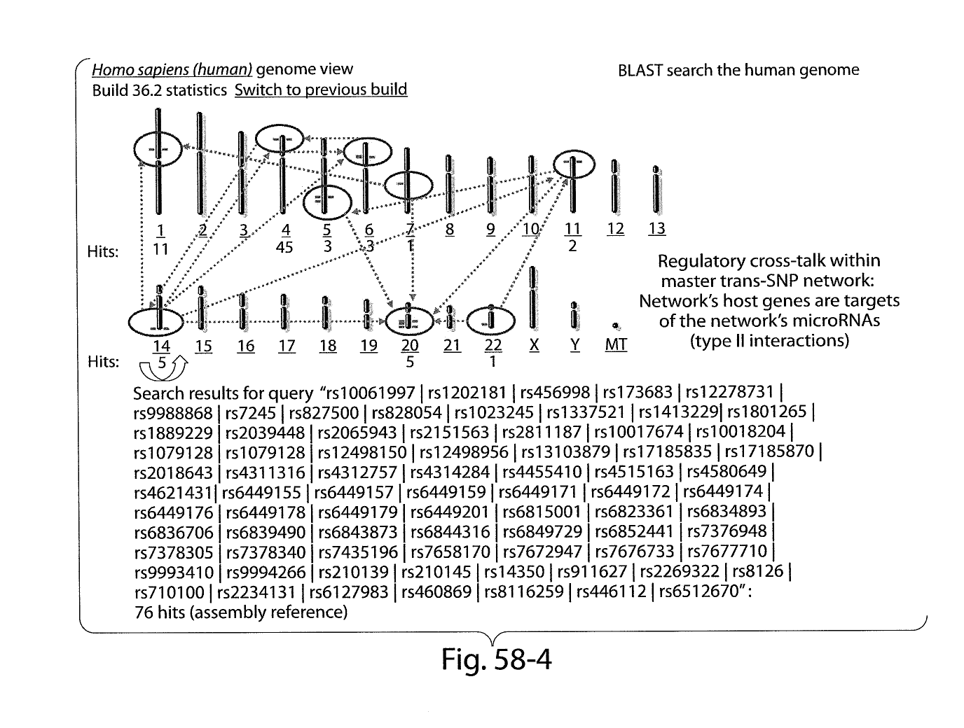

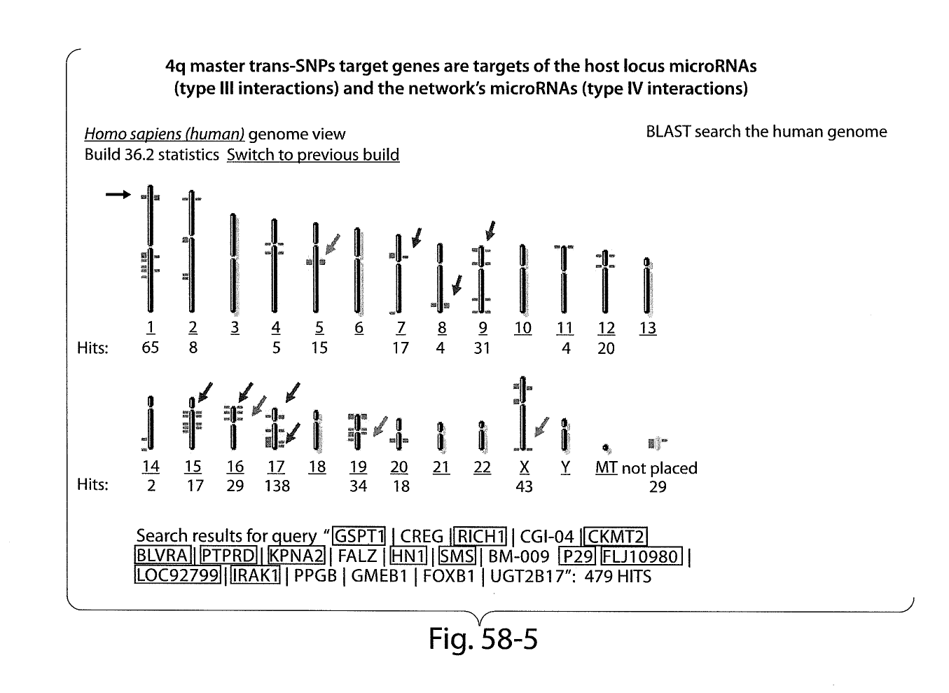

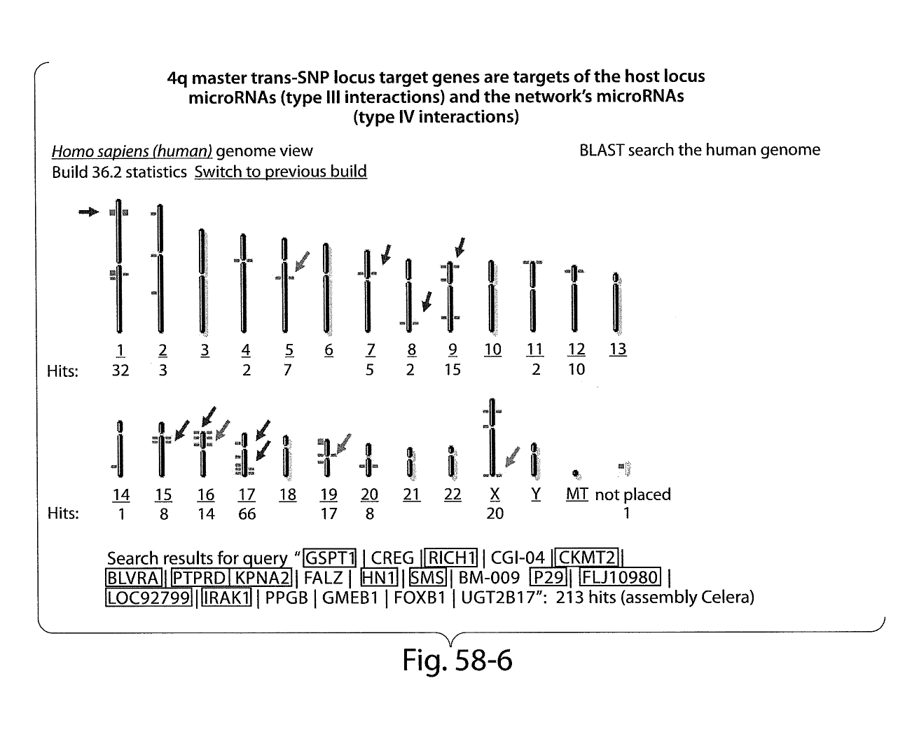

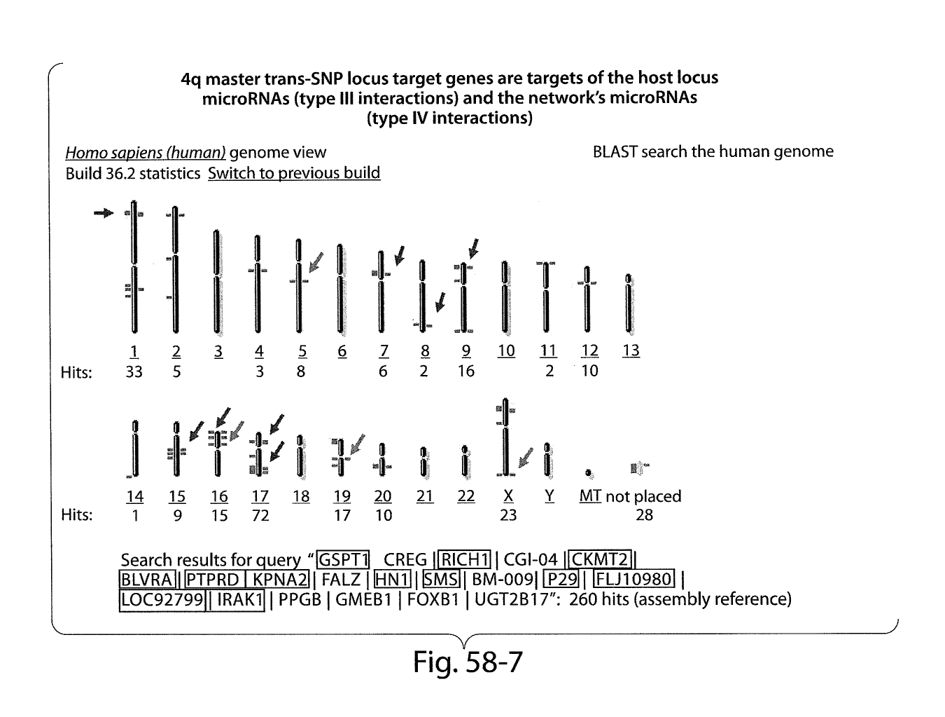

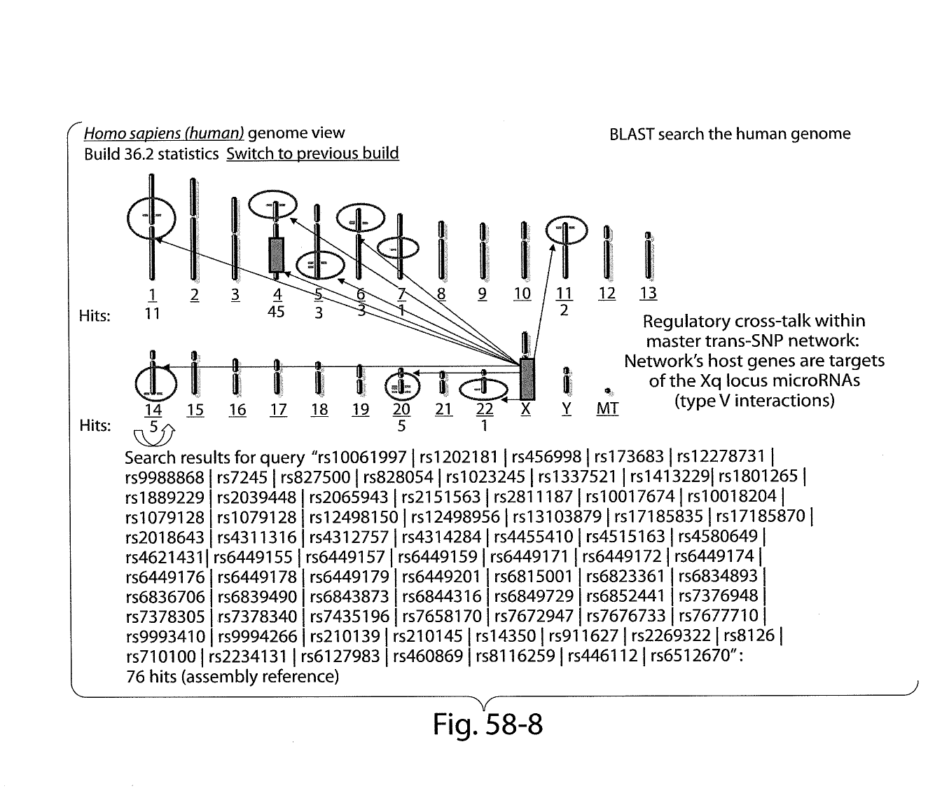

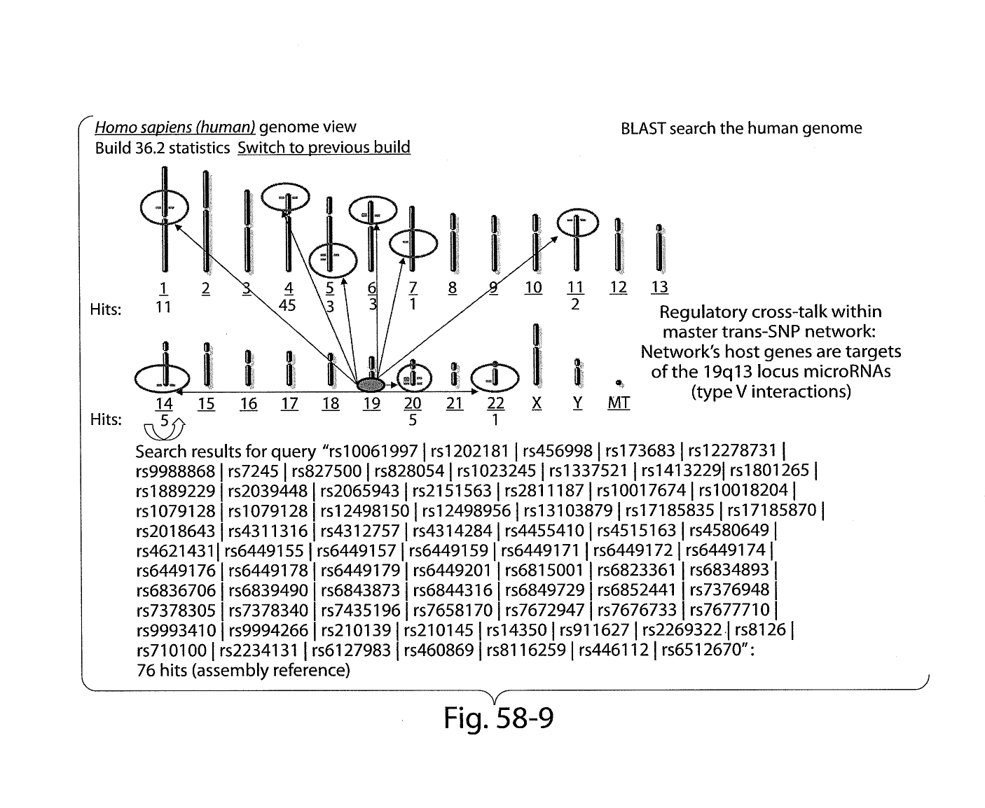

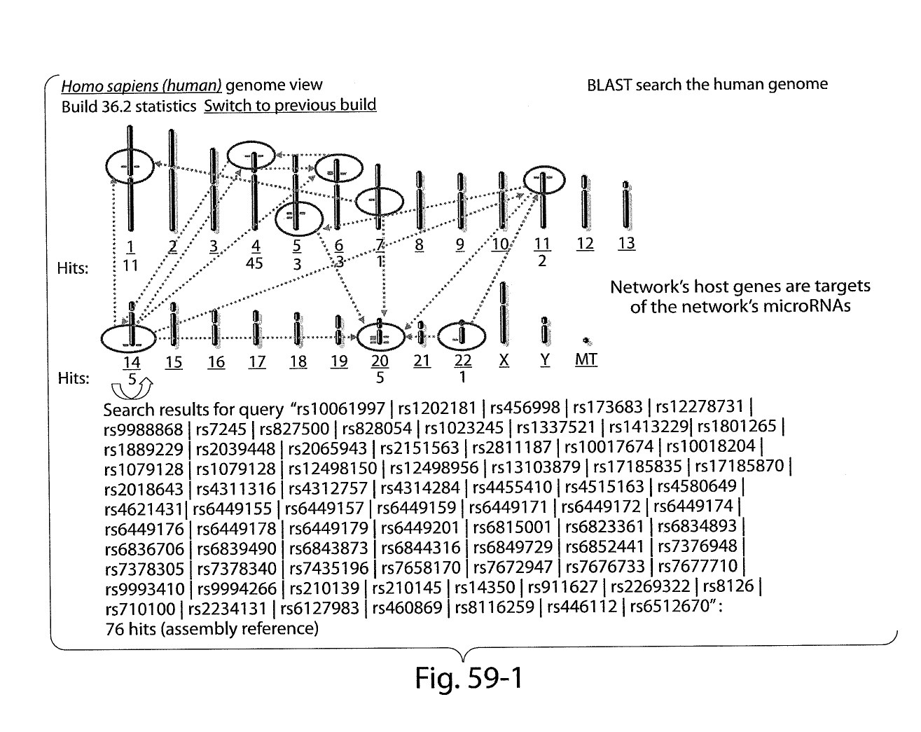

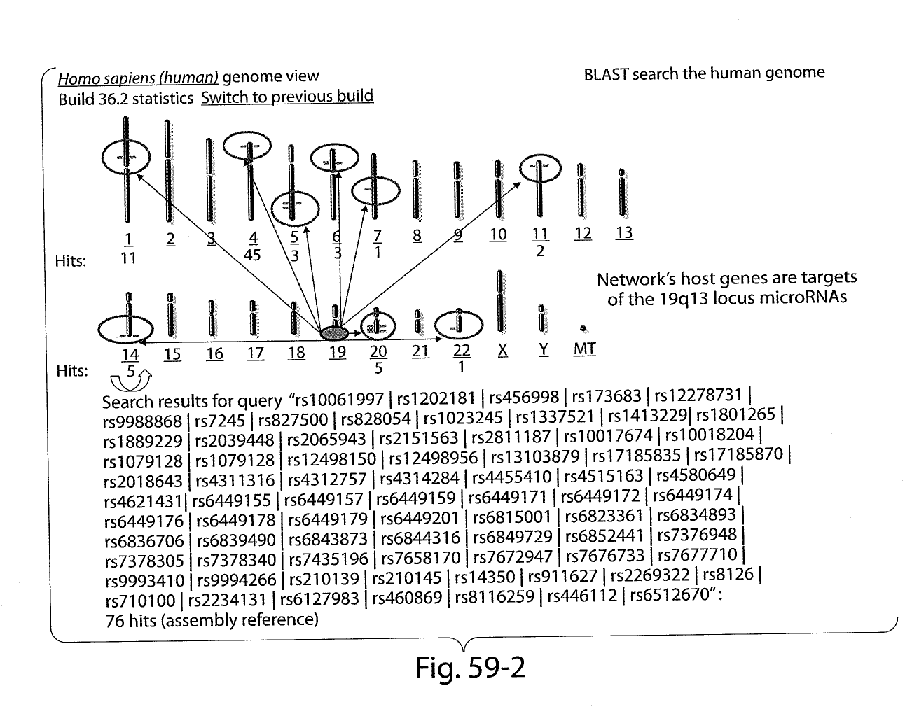

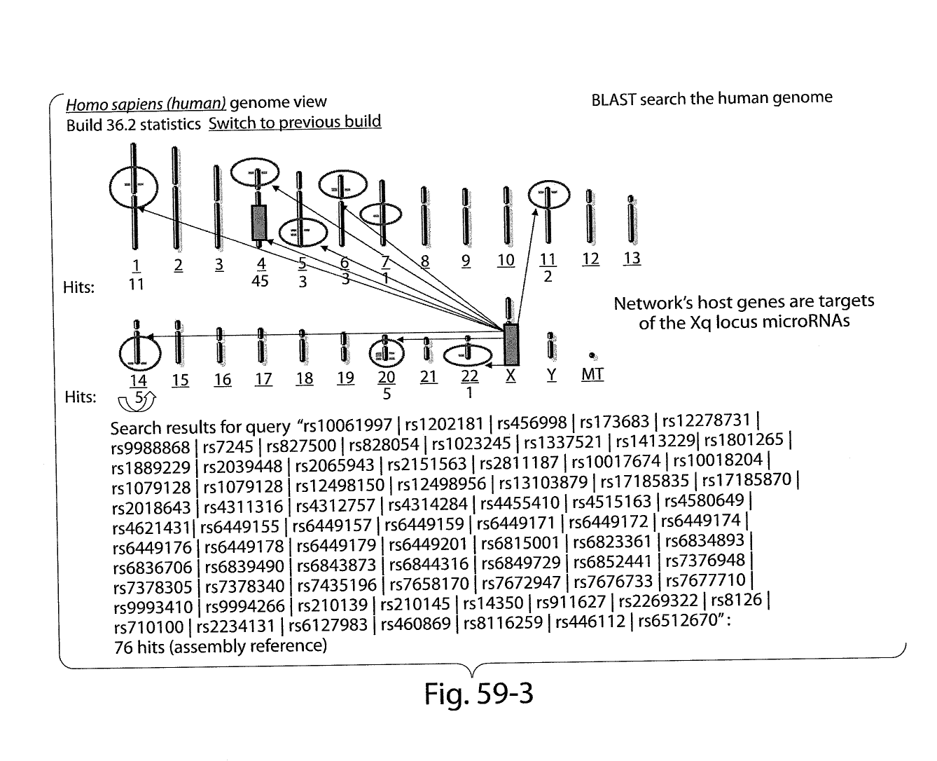

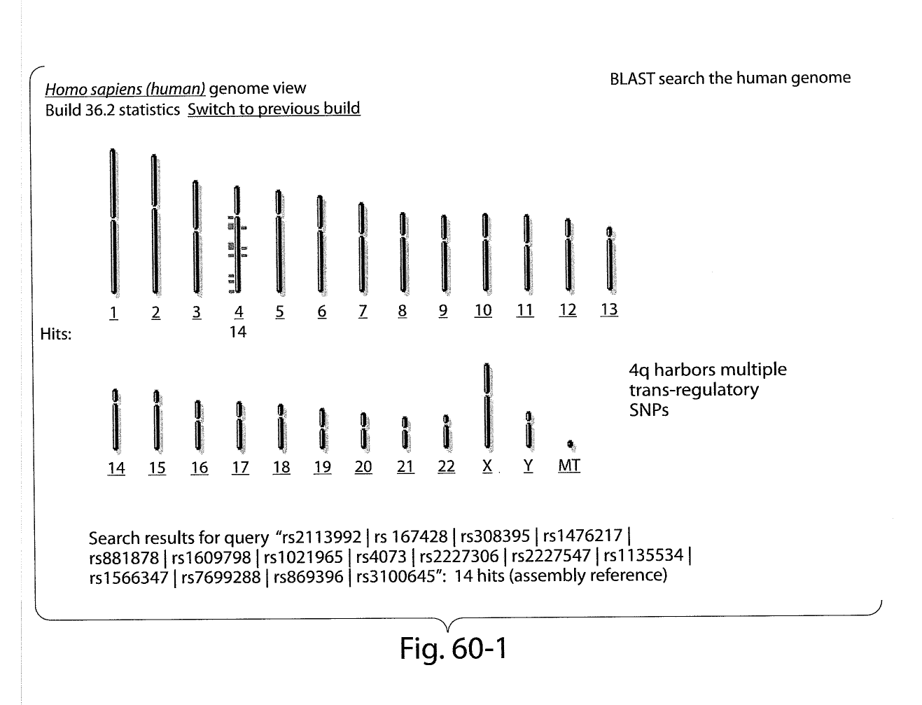

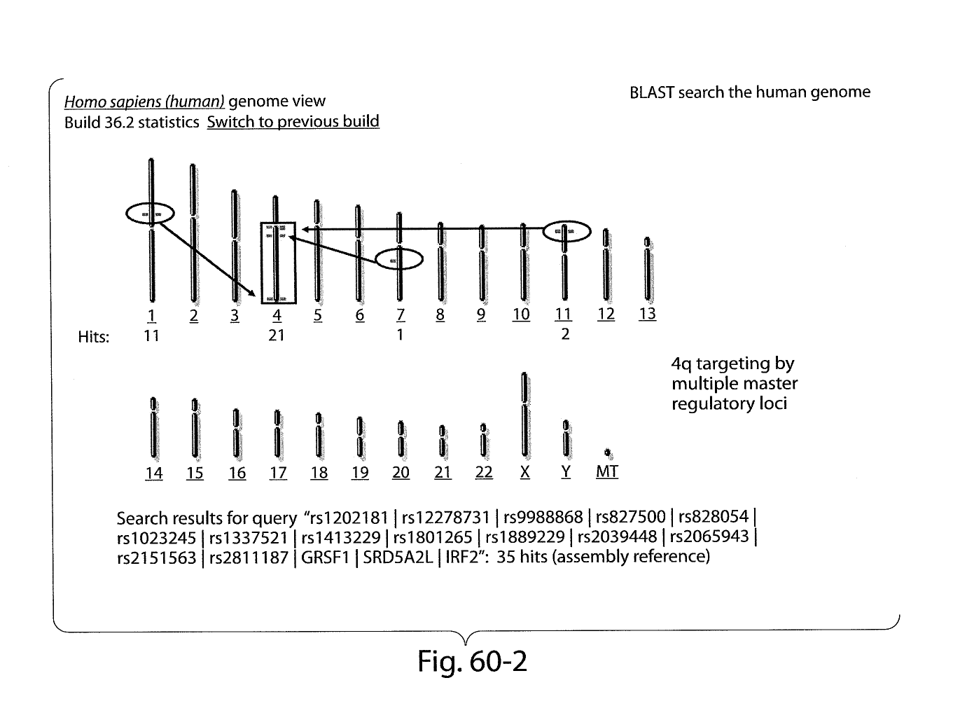

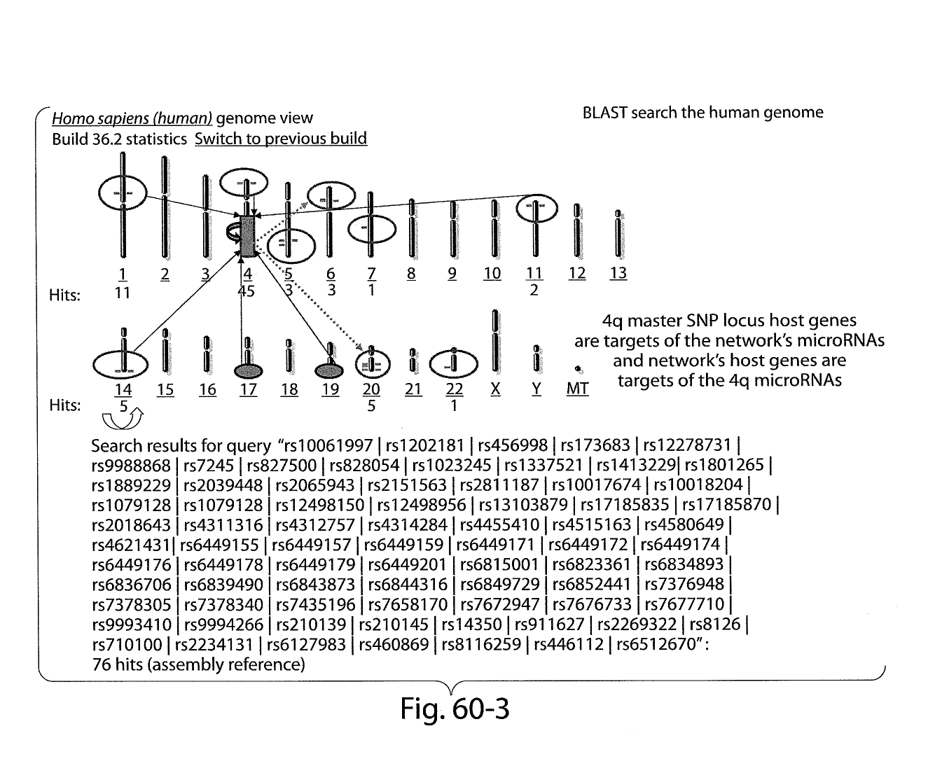

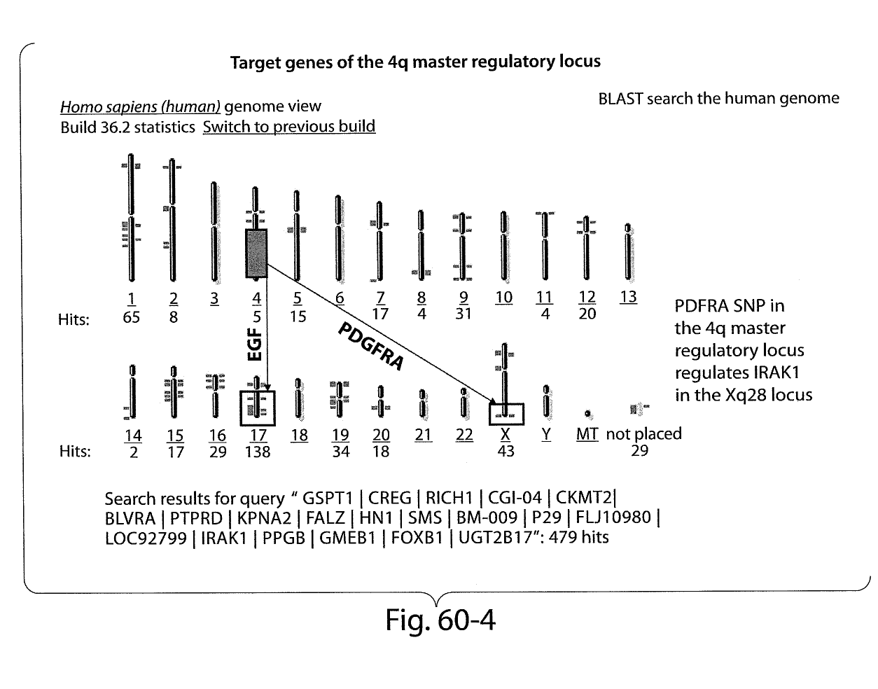

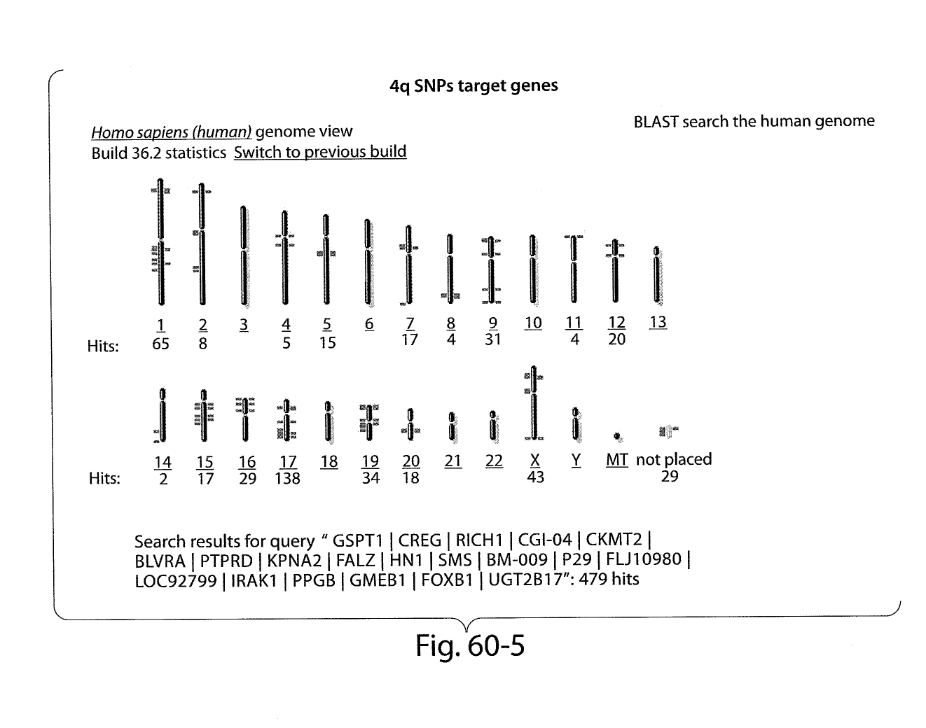

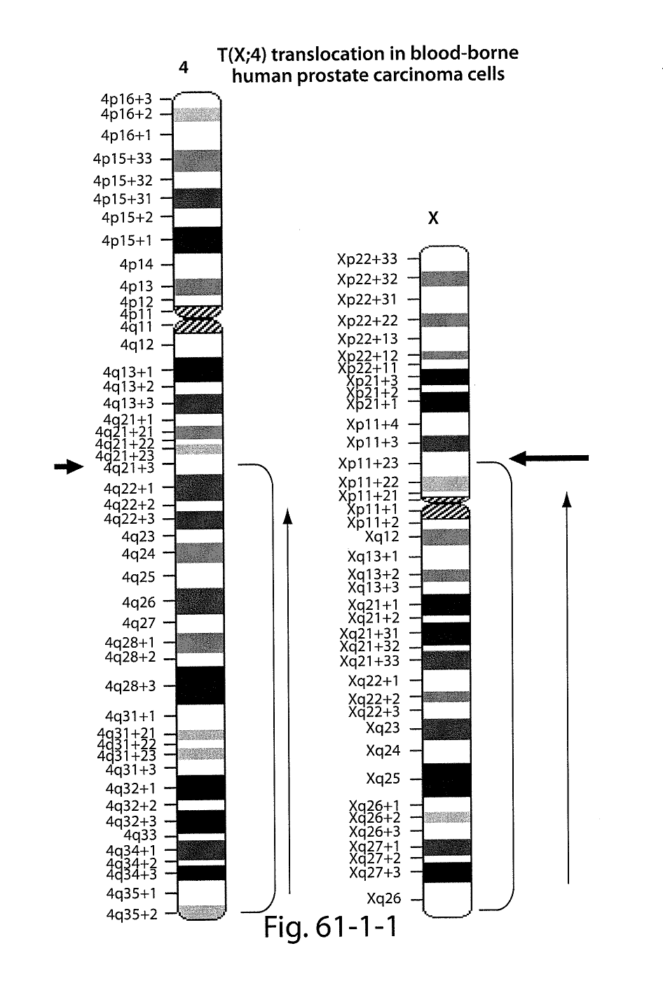

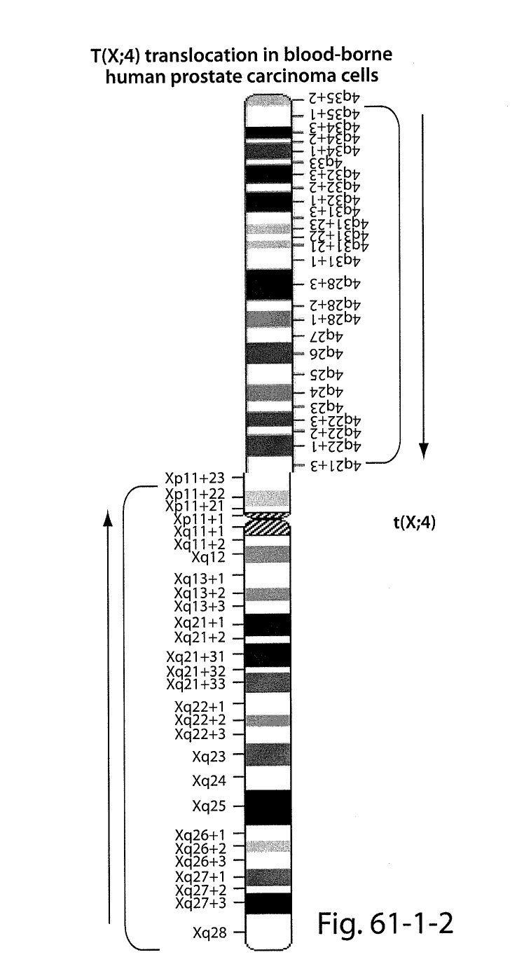

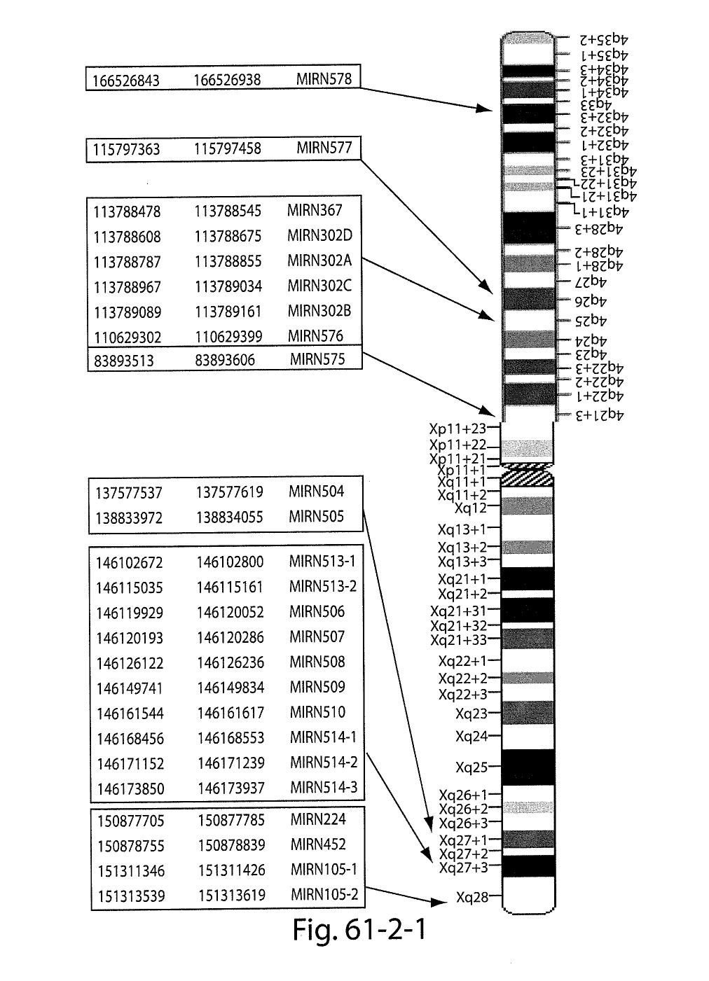

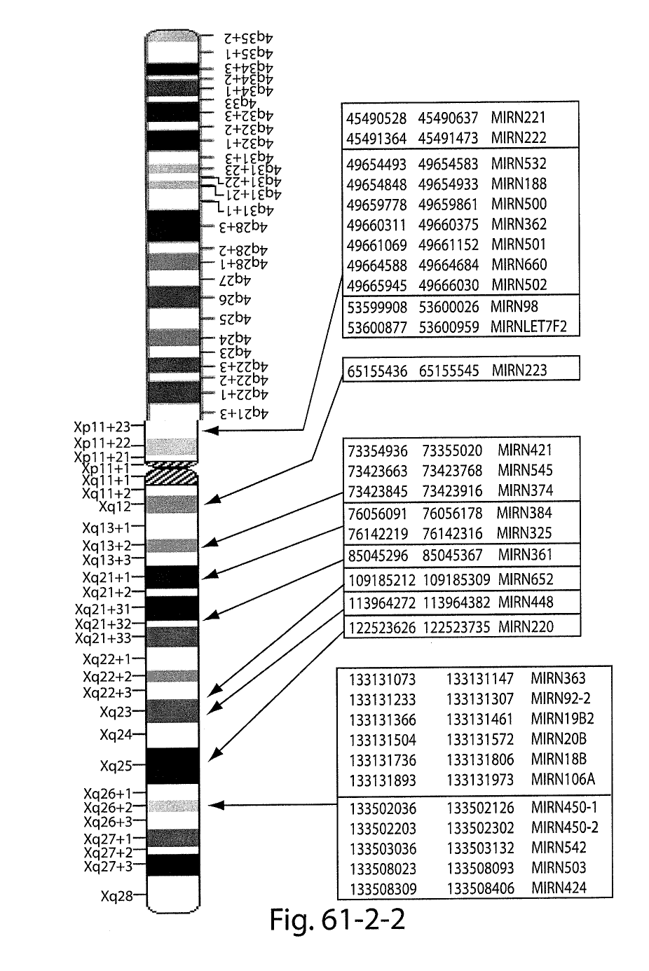

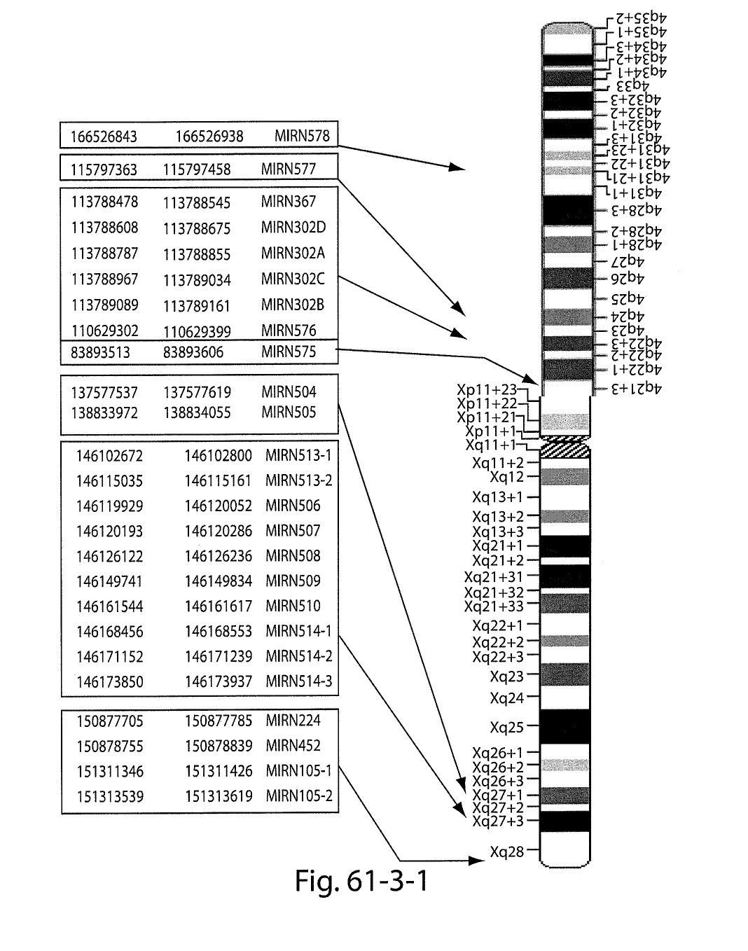

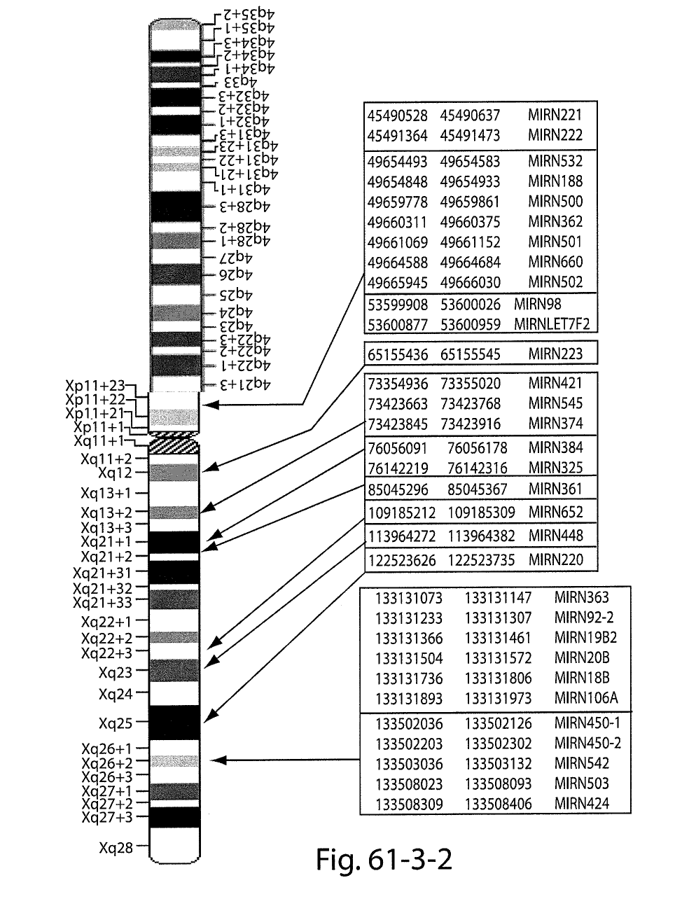

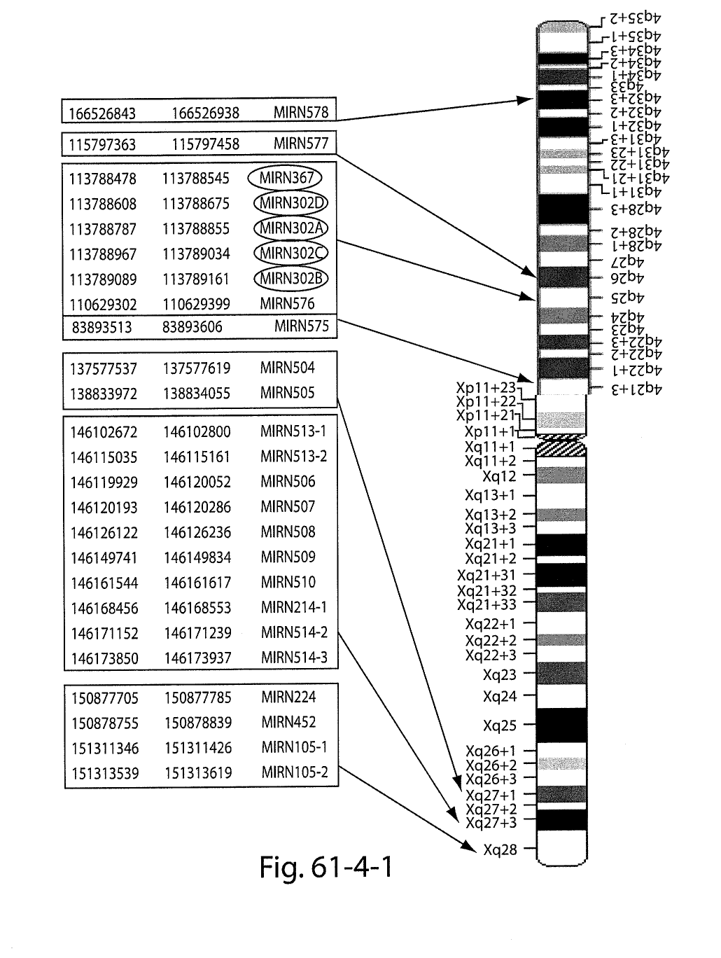

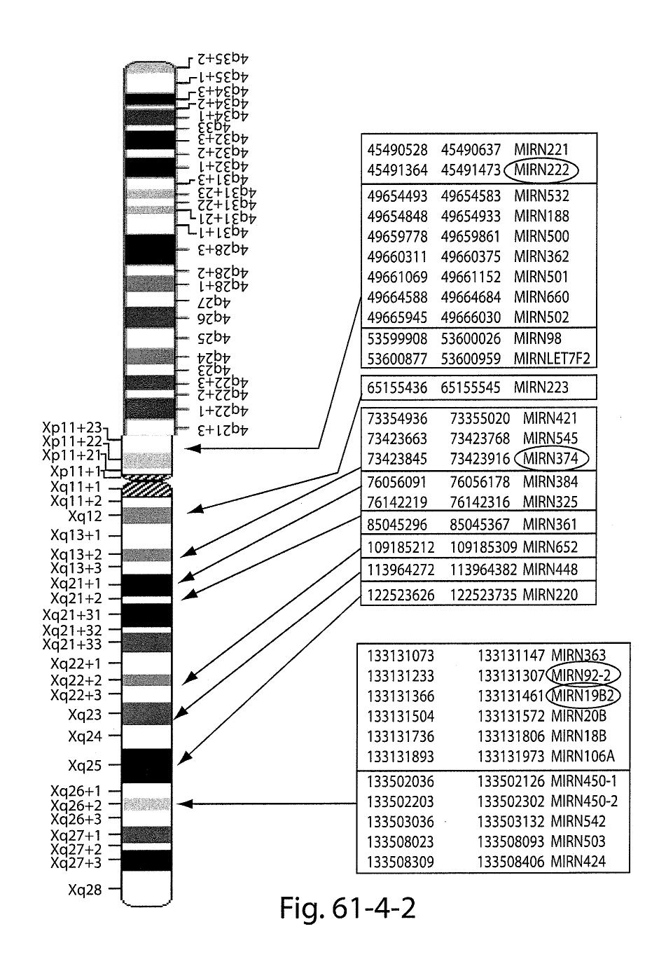

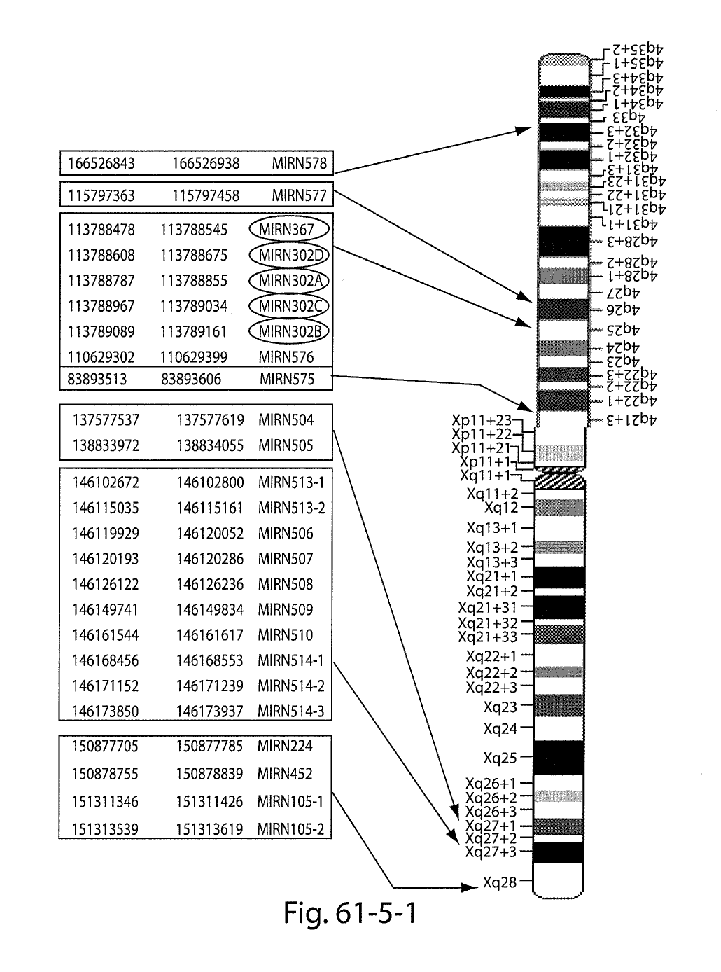

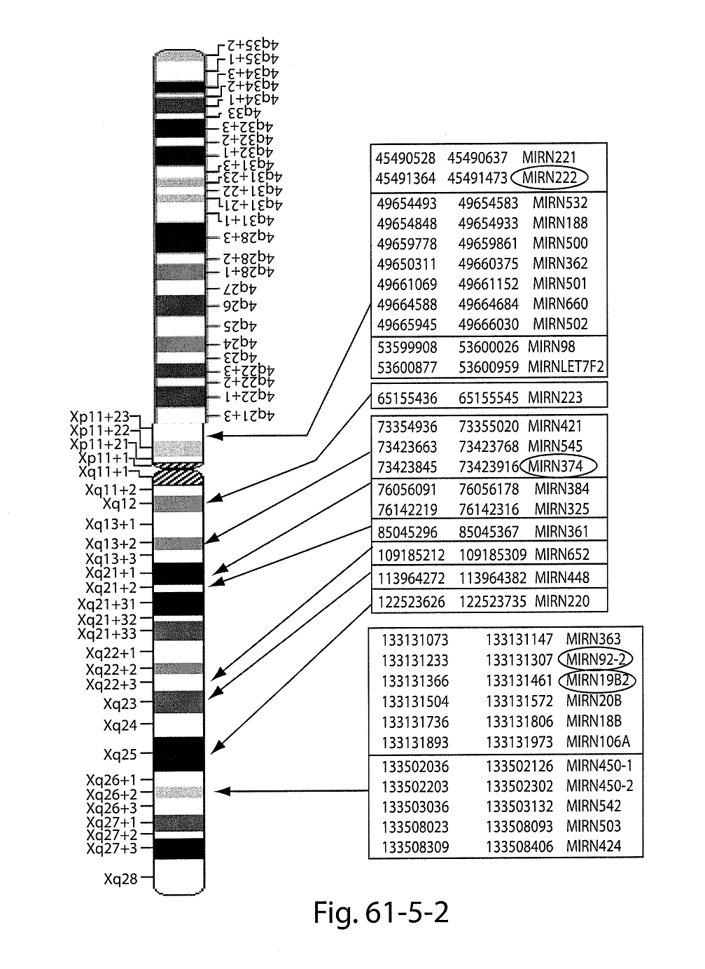

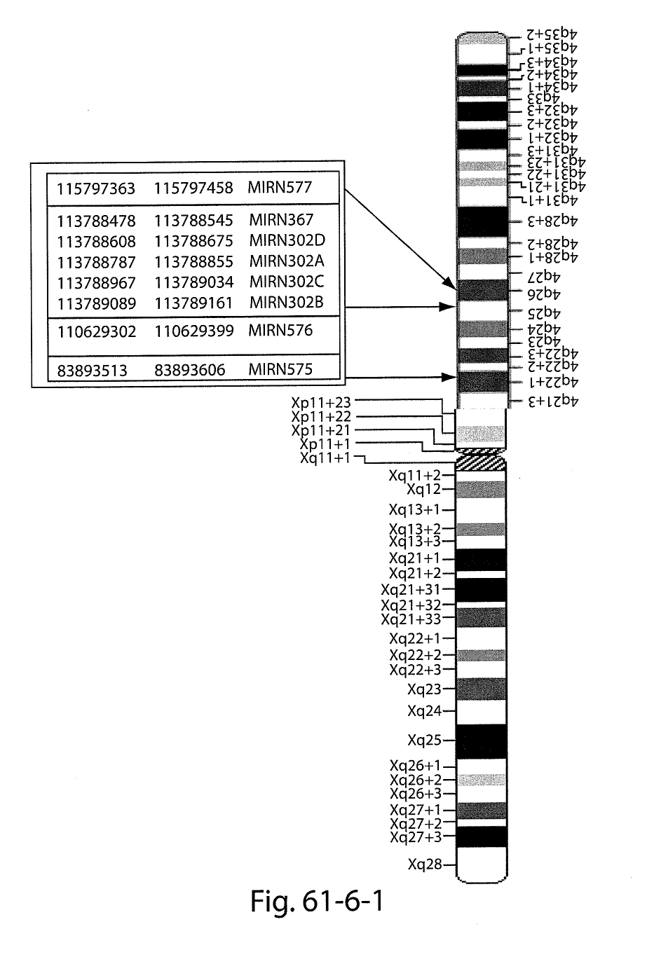

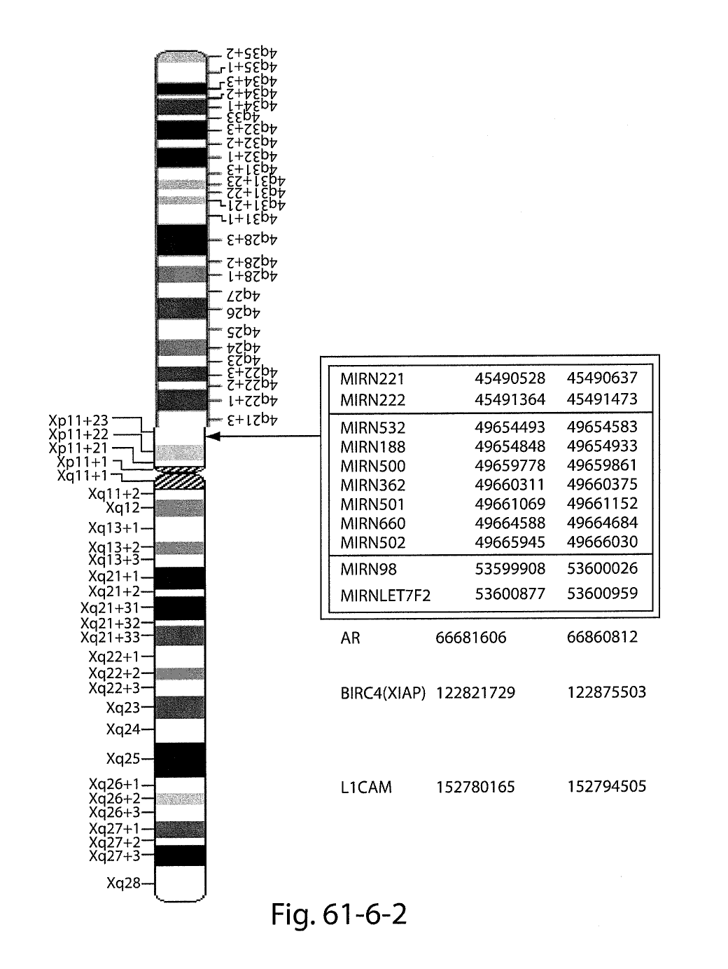

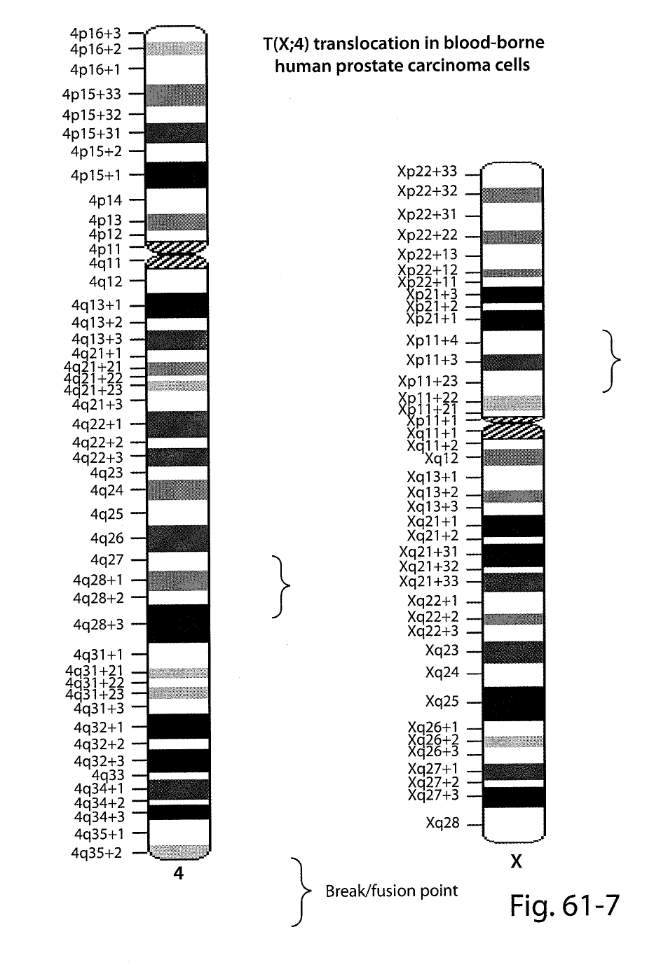

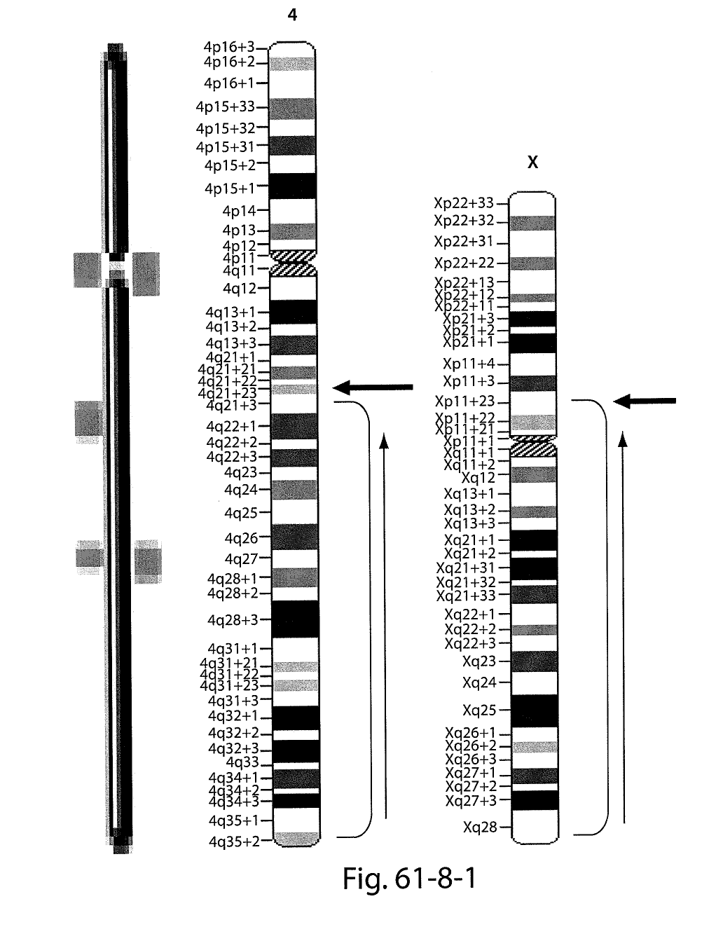

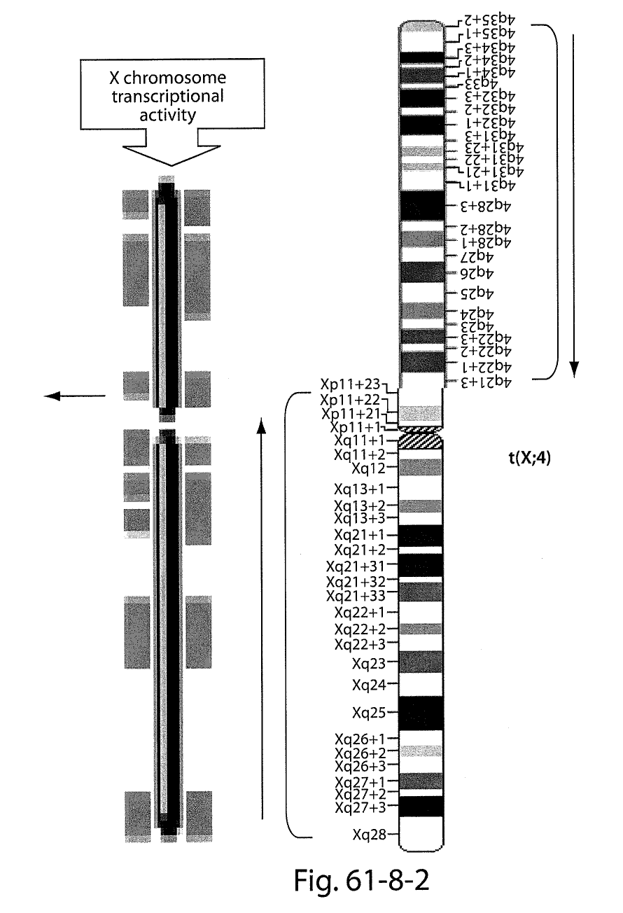

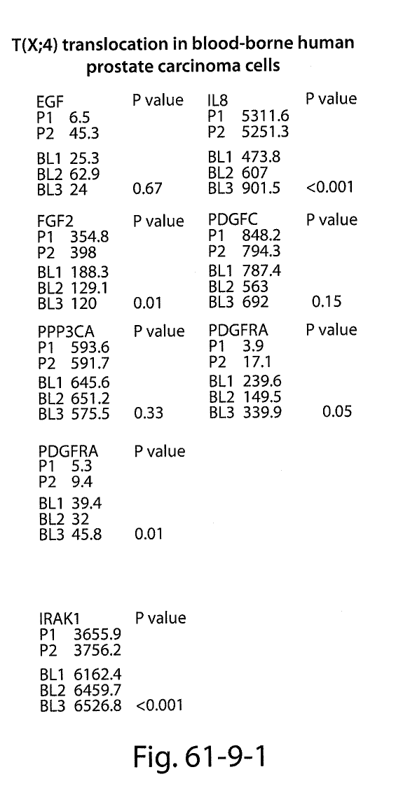

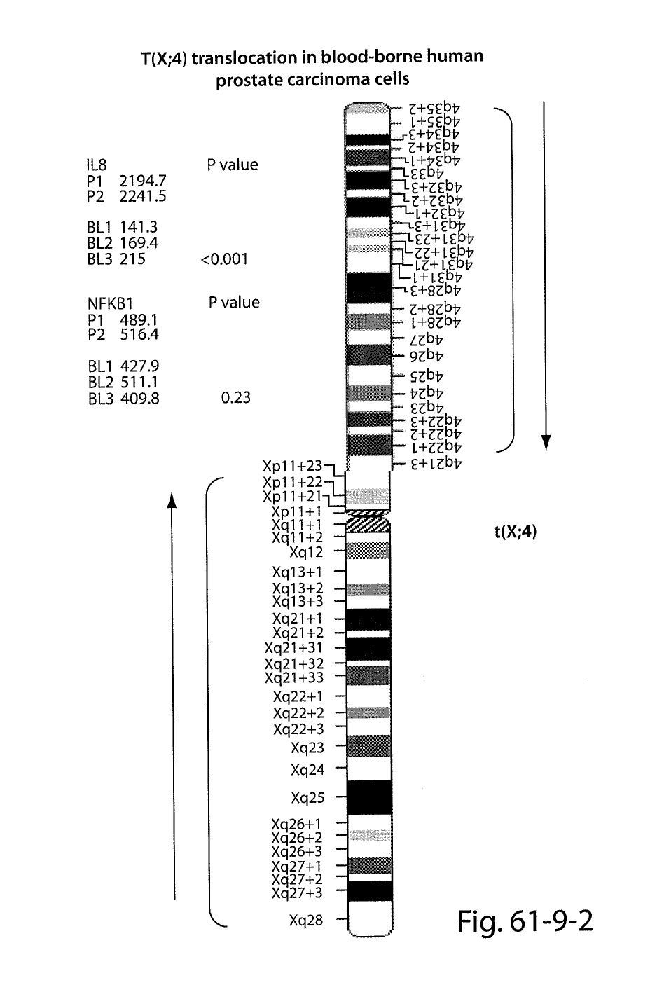

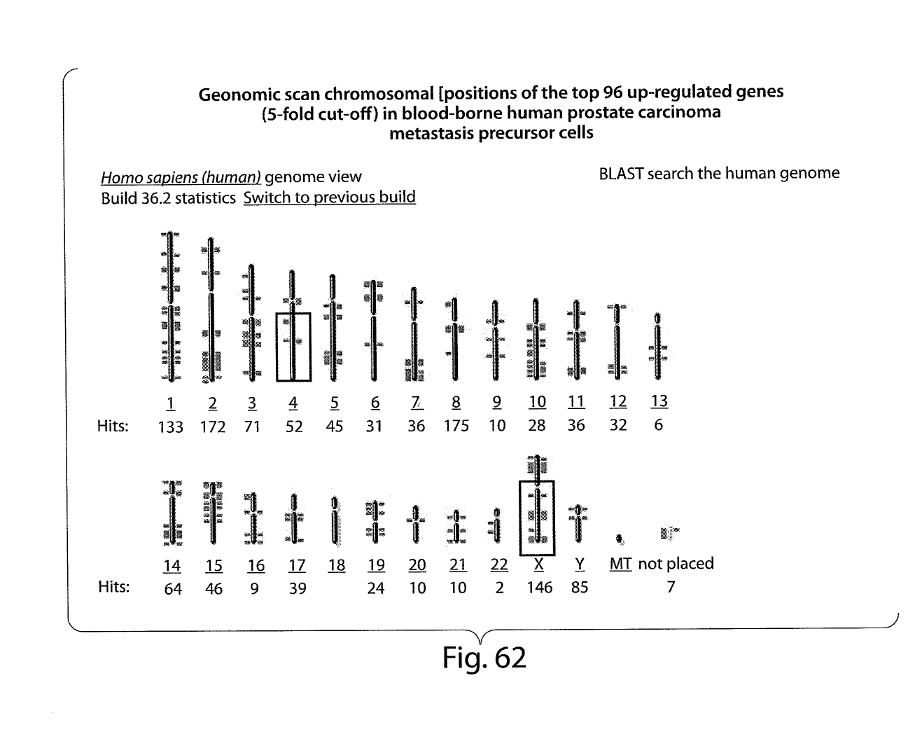

Taken together, these observations support the concept of a trans-SNP/microRNA master regulatory network. One of the main operational features of this network is microRNA signaling and intron/exon cross-talk between transcripts derived from SNP sequences of network's host genes and microRNAs aiming at network's target genes. Six types of informational and potential regulatory interactions within the trans-SNP/microRNA master regulatory network were defined (See, FIG. 58):
Type I interactions reflect associations between SNP variations and gene expression changes (they define the coordinates of the given regulatory locus, regulatory SNP host gene and target genes, as well as interacting regulatory loci comprising the regulatory network);
Type II interactions reflect potential regulatory effects of host regulatory locus microRNAs (microRNAs residing in close proximity to MTSRs) on SNP host genes;
Type III interactions reflect predicted effects of host regulatory locus microRNAs on SNP target genes;
Type IV interactions reflect potential regulatory effects of network's “hub” microRNAs (residing in close proximity to genetic loci targeted by multiple network's SNPs) on network's host genes;
Type V interactions reflect effects of network's “hub” microRNAs on network's target genes;
Type VI interactions reflect the Watson-Crick base pairing-mediated effect reflecting sequence homology between master trans-SNPs and microRNAs;

A simple theoretical model can be envisioned demonstrating how these interactions based solely on RNA/RNA communications would integrate all 12 MTSRs into a highly interconnected gene expression regulatory network comprising 23 host genes; 89 regulatory SNPs; 163 SNP target genes; and 227 microRNAs. The postulated main regulatory signals driving the functional integration of this network and the feed-forward communications between distinct MTSRs are based on predicted competitive interactions between microRNAs, mRNAs, and non-coding RNAs with common target sequences. Sequence homology profiling analysis supports the concept of the trans-SNP/microRNA master regulatory network operating via microRNA signaling and intron/exon cross-talk between SNP host genes and microRNA target genes. Intriguingly, many chromosomal components of this regulatory network were previously defined as chromosomal regions frequently targeted for palindrome-driven DNA amplification in human cancers as well as common malignancy-associated regions of recurrent transcriptional activation (MARTA) in human breast, prostate, ovarian, and colon cancers.

Evolutionary Consequences of the Genome-Scale Pervasive Transcription

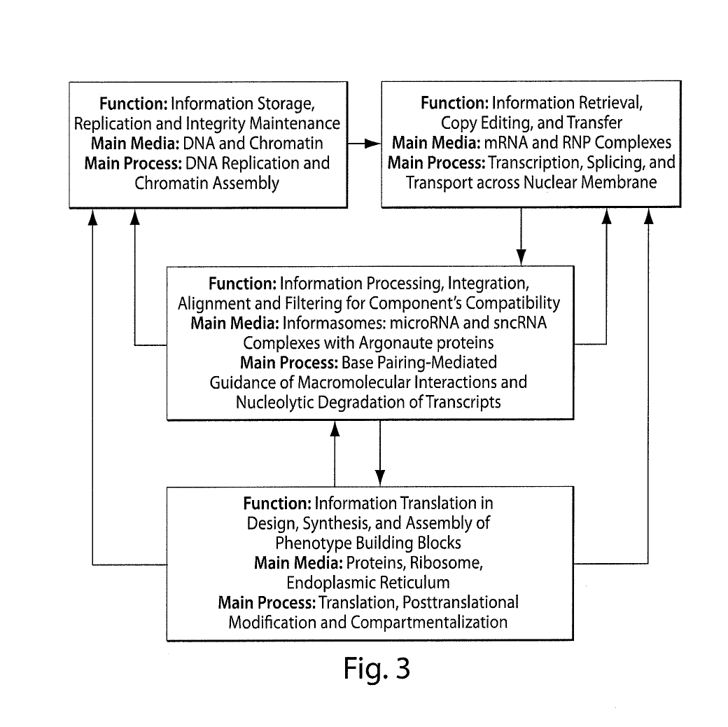

Recent studies demonstrate the enormous complexity of the human transcriptome generating the vast amount of RNA transcripts from alternative splicing and protein coding and non-protein-coding DNA sequences. It has been suggested, that the remarkable diversity of RNA species of the human transcriptome coupled with multitude of its regulatory functions and structural features may help find the answer to the “genome complexity conundrum” by explaining the dramatic increase of regulatory complexity and phenotypic variations in higher eukaryotes despite having similar numbers of protein coding genes. An information-centered model of a cell suggesting that informasomes represent the intracellular structures which provide the increasingly complex framework of genomic regulatory functions in higher eukaryotes has been proposed (See, FIG. 3).

Analysis of novel TARs as well as some random regions of the genome indicates that much of the human genome produces transcripts that are present in the polyA+ RNA form, at least at a level of 10⁻⁸ to 10⁻¹⁰ in total RNA. The finding that much of the genome is likely to be expressed and that RNA is translated has been previously reported for yeast. (See, Ross-Macdonald, et al., Large-scale analysis of the yeast genome by transposon tagging and gene disruption. Nature 402:413-418 (1999) and Coelho P S, et al., Genome-wide mutant collections: toolboxes for functional genomics. Curr. Opin. Microbiol. 3:309-315 (2000)). It has been suggested, that the ability to continuously express novel regions of the genome could ultimately be useful in evolution for selecting new functionally beneficial sequences. Moreover, it may provide an evolutionary-compatible mechanism of generation of the subtle incremental combinatorial variations of gene expression without dramatic alterations of the phenotype and overall “fitness” of an organism.

One of the remarkable consequences of the genome-scale pervasive transcription and translation would be the generation of highly specific individual genomic scans of nearly all possible combinations of peptide sequences which are uniquely tailored to the individual's DNA sequence variations including specific SNP patterns. It can be envisioned as a critical component of the pervasive transcription- and translation-driven mechanisms of the ontogenesis of immunological competence including mechanisms of self/non-self discrimination and tolerance. A recent study concluded that it is likely that many (and possibly the majority) of known protein-coding genes are expressed and spliced in most human tissues and cell lines and that multiple transcripts are produced from most gene loci at least at a low level, suggesting that these conclusions are valid for antigen-presenting cells as well. (See, Wu J Q, et al., Systematic analysis of transcribed loci in ENCODE regions using RACE sequencing reveals extensive transcription in the human genome, Genome Biol. 9:R3 (2008)).

For many years, the understanding of biological systems was shaped by strictly deterministic lock-and-key types of models which were influenced by the astonishing beauty of the enzyme-substrate interactions and the remarkable molecular precision of the Watson-Crick pairing. However, it turns out that many critically important biological processes are most likely relying on stochastic (random and probabilistic) mode of actions. (See FIG. 3). Thus, stochastic rather than deterministic mode of choices in a sequence of events essentially eliminates the probability of phenotype duplication during ontogeny and creates a base for the infinite phenotype diversity during adaptation. It also presents an enormous challenge in the quest to understand how the RNA universe-guided affinity-driven interactions between macromolecules affect the probability of regulatory choices defining phenotypes.

The sequence homology profiling of 2301 human small non-coding RNAs transcript that were previously identified and are accessible in publicly available databases was carried out as shown in Example 1, infra.

### A SNP-Guided MicroRNA Map of Six Common Human Disorders Identifies a Consensus Disease Phenocode Aiming at a Single Target Gene

Molecular definition of the mechanistic links of genetic variations to disease phenotypes remains one of the most formidable obstacles to understanding the underlying mechanisms of common human disorders. Recent large-scale high-powered genome-wide association (GWA) studies identified SNP variants manifesting highly significant associations with many common human disorders, which strongly imply that these genetic variations may have the potential causal effects on phenotypes of several major human diseases. These carefully designed studies identified highly significant genetic variants (e.g. SNPs), which are associated with disease phenotypes at the unprecedented levels of statistical confidence and supported by convincing replication. Therefore, it is highly likely that identified genetic traits may contribute to pathogenesis of human disorders, and this knowledge will enable the precise molecular understanding of how genetic variations contribute to pathological phenotypes. Mechanistic considerations of candidate genetic loci contributing to disease pathogenesis were limited to protein-coding genes within or near physical boundaries of which these genomic variants and SNPs are located. Most recently, this approach was extended to include the SNP variants residing within boundaries of genes encoding microRNAs and SNPs within microRNA-target sites in mRNAs (a concept known as the Patrocles polymorphism). A majority of most significant disease-linked SNPs identified to date is located within introns or non-genic regions of human genomes, which have no direct relations to known protein-coding sequences or microRNA genes, suggesting that non-canonical mechanisms of phenotype-altering effects of genetic variations may be relevant.

The idea that variations in DNA sequences associated with multiple major human disorders may affect phenotypes in trans, namely via non-protein-coding RNA intermediaries interfering with the biogenesis and/or functions of microRNAs, was tested. It was reasoned that, if RNA transcripts have the potential to interfere with the biogenesis or bioactivity of microRNAs, they must exhibit the apparent sequence homology/complementarity features to the targeted microRNAs. The analysis revealed a systematic primary sequence homology/complementarity-driven pattern of associations between disease-linked SNPs, microRNAs, and protein-coding mRNAs defined here as a human disease phenocode. Specifically, a human disease phenocode of 72 SNPs and 18 microRNAs with an apparent targeting bias to mRNA sequences derived from a single protein-coding gene, KPNA1, was uncovered. Each of the microRNAs in this elite set appears linked to at least three common human diseases and has potential protein-coding mRNA targets among the principal components of the nuclear import pathway suggesting that genetic and molecular pathology of the nuclear import pathway contributes to pathogenesis of many common human disorders. Remarkably, practical application of this concept reveals a common phenocode for six major human disorders namely bipolar disease (BP); rheumatoid arthritis (RA); coronary artery disease (CAD); Crohn's disease (CD); type 1 diabetes (T1D); and type 2 diabetes (T2D). A consensus human disease phenocode comprises 29 SNPs and 10 microRNAs with an apparent propensity to target mRNA sequences derived from a single protein-coding gene, KPNAI.

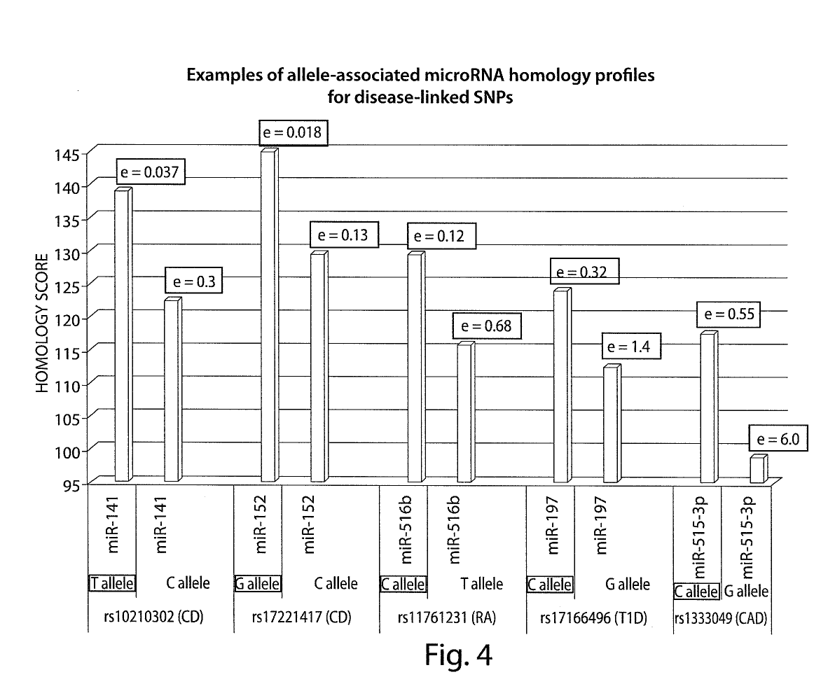

It was reasoned that, if RNA transcripts have the potential to interfere with the biogenesis or bioactivity of microRNAs, they must exhibit the apparent sequence homology/complementarity features to the targeted microRNAs. To test the validity of this concept, the sequence homology profiling of 81 SNPs was carried out using those SNPs that are most significantly associated with seven common human disorders, namely bipolar disease (BP); rheumatoid arthritis (RA); coronary artery disease (CAD); Crohn's disease (CD); type 1 diabetes (T1D); type 2 diabetes (T2D); and hypertension (HT). It was found that 77 of 93 SNP sequences (83%) manifest homology or complementarity to 153 human microRNAs exceeding the default level of statistical threshold for the e-value of 10. Interestingly, a majority of SNPs (12 of 16; 75%) with no detectable homology to human microRNAs at the default level of significance was derived from the SNPs with moderate disease association levels, suggesting that SNPs with the strongest disease association are enriched for sequences homologous to the microRNAs. Consistently, 90% of SNPs (34 of 38) with the most significant disease associations manifest sequence homology to human microRNAs compared to 78% of SNPs (43 of 55) with the moderate disease association levels. It was noted that, in many instances, the profiles of sequence homology interactions between SNPs and microRNAs manifest distinct allele-specific patterns (See FIG. 4), which is consistent with the postulated regulatory and/or disease-causing functions of these sequences.

An elite set of 10 microRNAs which have at least 3 sequence homology counterparts among 29 top-scoring disease-associated SNPs was identified (Table 1).

TABLE 1
29 SNPs and 10 microRNAs comprising a consensus phenocode of six common human diseases

| | | microRNA/ | | | | | | | | | | | |
|---|---|---|---|---|---|---|---|---|---|---|---|---|---|
| | SNP | reported | | miR- | | | miR- | miR- | | | | | P value, |
| Disease | Chromosome | SNP | mir-125 | 181 | miR-29 | mir-519 | 541 | 147 | mir-199 | miR-297 | mir-520 | mir-558 | SNP |
| BD | 16p12 | rs420259 | | | 1 | | | | | | | | 0.00058 |
| BD | 9q32 | rs10982256 | | | | | | | | 1 | | | 0.00058 |
| BD | 14q32 | rs11622475 | | | | | | | 1 | | | | 0.00058 |
| BD | 8p12 | rs2609653 | | | | | | 1 | | | | | 0.00058 |
| BD | 6p21 | rs6458307 | | | 1 | | | | | | | | 0.00058 |
| CAD | 9p21 | rs1333049 | | | | 1 | | | | | 1 | | 1.5E−07 |
| CAD | 5q21 | rs383830 | | | | | | | | | | 1 | 0.00058 |
| CD | 5q33 | rs1000113 | | | | | | | | | | 1 | 0.00058 |
| CD | 1p31.3 | rs11209026 | 1 | | | | | | 1 | | | | 1.5E−07 |
| CD | 1p31 | rs11805303 | | | | 1 | | | | | | | 0.00058 |
| CD | 5p13 | rs17234657 | | | | | | 1 | | | | | 0.00058 |
| CD/T1D | 18p11 | rs2542151 | 1 | | | | | | | | | | 0.00058 |
| CD | 6p22 | rs6908425 | | 1 | | | | | | | | | 0.00058 |
| CD | 7q36 | rs7807268 | 1 | | | | | | | | | | 0.00058 |
| CD | 6p21 | rs9469220 | | 1 | | | | | | | | | 0.00058 |
| RA/TID | 1p13.3-p13.1 | rs2476601 | | | | | 1 | | | | | | 0.00058 |
| RA | 6p21.3 | rs615672 | | | | | 1 | | | | | | 0.00058 |
| RA | 6 MHC | rs6457617 | | | | | | | | 1 | | | 0.00058 |
| RA | 1p36 | rs6684865 | | 1 | | | | | | | | | 0.00058 |
| RA | 6p23 | rs6920220 | | | | 1 | | | | | | | 0.00058 |
| RA | 13q12 | rs9550642 | | | | | | | 1 | | | | 0.00058 |
| T1D | 12p13 | rs3764021 | | | | | | 1 | | | | | 0.00058 |
| T1D | 2q24 | rs3788964 | | | 1 | | | | | | | | 0.00058 |
| T1D | 12p13 | rs11052552 | | | | | | | | 1 | | 1 | 1.5E−07 |
| T1D | 1q42 | rs2639703 | | | 1 | | | | | | | | 0.00058 |
| T2D | 3p25 | rs1801282 | | | | 1 | | | | | 1 | | 1.5E−07 |
| T2D | 1p31 | rs4655595 | | 1 | | | | | | | | | 0.00058 |
| T2D | 11p15.1 | rs5219 | | | | | 1 | | | | | | 0.00058 |
| T2D | 10q25.3 | rs7903146 | | | | | | | | | 1 | | 0.00058 |
| | | SCORE | 3 | 4 | 4 | 4 | 3 | 3 | 3 | 3 | 3 | 3 | |
| | | P value, miR | 0.00638 | 0.00045 | 0.00045 | 0.00045 | 0.00638 | 0.00638 | 0.00638 | 0.00638 | 0.00638 | 0.00638 | |

Legend:
Abbreviations:
BD, bipolar disease;
CAD, coronary artery disease,
CD, Crohn's disease,
RA, rheumatoid arthritis;
TID, type 1 diabetes;
T2D, type 2 diabetes.
Numbers (1) in the table indicate the SNPs with sequence homology to corresponding microRNAs.
The score values represent the total number of SNPs with sequence homology to a given microRNA.
500000 SNPs were analyzed for associations with common human diseases to indentify 81 SNPs with most significant associations (1). Sequences homology profiling of 81 SNPs identified 29 SNPs with multiple call events of sequence homology to at least 3 microRNAs which are listed in the Table 1. To estimate the liklihood of the occurrence of multiple homology call events by chance, we carried out the hypergeometric distribution test and calculated the corresponding P values.

The probability that multiple sequence homology calls occurred by chance was estimated and it was found to be highly unlikely (Table 1). The sequence homology-driven associations of disease-linked SNP and microRNAs as shown in Table 1 is designated an SNP-guided microRNA map (“MirMap”) of human diseases. It was then determined whether the identified set of 10 microRNAs would have the potential to target a common group of mRNAs. Lists of predicted mRNA targets for each of the 10 microRNAs shown in Table 1 were retrieved using TargetScan database and searched for concordant sets of mRNA targets. Remarkably, the analysis reveals that 70% of the microRNAs identified in Table 1 have the potential to target mRNA sequences derived from a single protein-coding gene, namely KPNAI (importin alpha 5; Table 2).

TABLE 2
Majority of the consensus phenocode microRNAs have targeting potentials
toward mRNAs encoded by the importin alpha 5 (KPNAI) gene

| Targeted | | | | | | |
|---|---|---|---|---|---|---|
| importins/ | | | | | | |
| microRNAs | KPNA1 | KPNA2 | KPNA3 | KPNA4 | KPNA5 | KPNA6 |
| mir-125 | | | | | | mir-125 |
| miR-181 | miR-181 | | | miR-181 | | |
| miR-29 | miR-29 | | | miR-29 | | |
| mir-519 | mir-519 | | mir-519 | | | |
| miR-541 | miR-541 | | | | | |
| miR-147 | miR-147 | | | | | miR-147 |
| mir-199 | | | | mir-199 | | mir-199 |
| miR-297 | | | | | | |
| mir-520 | mir-520 | mir-520 | mir-520 | | | mir-520 |
| Score | 7 | 1 | 2 | 3 | 0 | 4 |
| P value | 0.006034 | 0.302798 | 0.284126 | 0.127604 | 0.68583 | 0.181507 |

Legend:
Human importin-targeting microRNAs were identified using TargetScan database. P values were calculated using hypergeometric distribution tests. They represent the estimates of the likelihood of obtaining score values by chance and take into account the numbers of all screened for homology microRNAs and the number of microRNAs which are predicted to target a given importin gene.

Moreover, 22 of 29 SNPs listed in Table 1 manifest sequence homology to microRNAs which are predicted to target KPNA1 gene, thereby indicating that sequence homology to the KPNA1-targeting microRNAs is a common structural (and, perhaps, functional) feature of many SNPs associated with multiple major human diseases.

To test whether KPNA1 targeting is specific, it was estimated that the predicted targeting effect by the consensus microRNAs on a distinct set of mRNAs, which are derived from five other importin-encoding genes and are functionally and structurally closely related to the KPNA1 gene. The predicted targeting effect on mRNAs of five distinct importins did not reach the threshold of statistical significance to exclude the likelihood of occurrence of multiple calls by chance. (See, Table 2). This suggests that the predicted KPNA1 mRNA targeting by the consensus microRNAs is specific. Thus, it is tempting to speculate that KPNA1 is the gene representing a common disease target in at least six major human disorders (BD; RA; CAD; CD; T1D; T2D). The sequence homology-driven associations of disease-linked SNPs, microRNAs, and mRNA target genes as a consensus phenocode of human diseases can be defined.

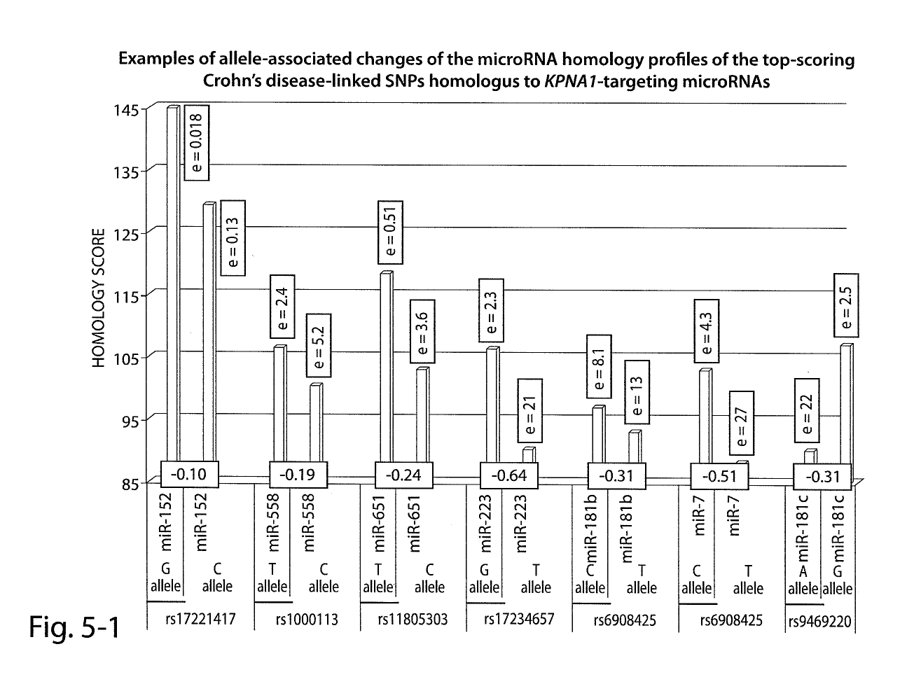

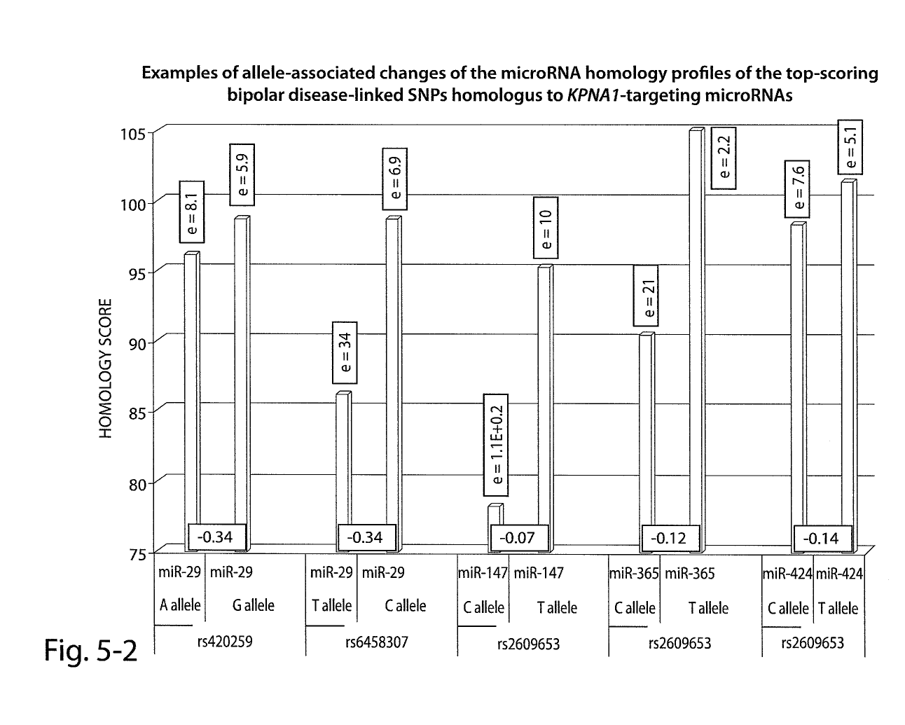

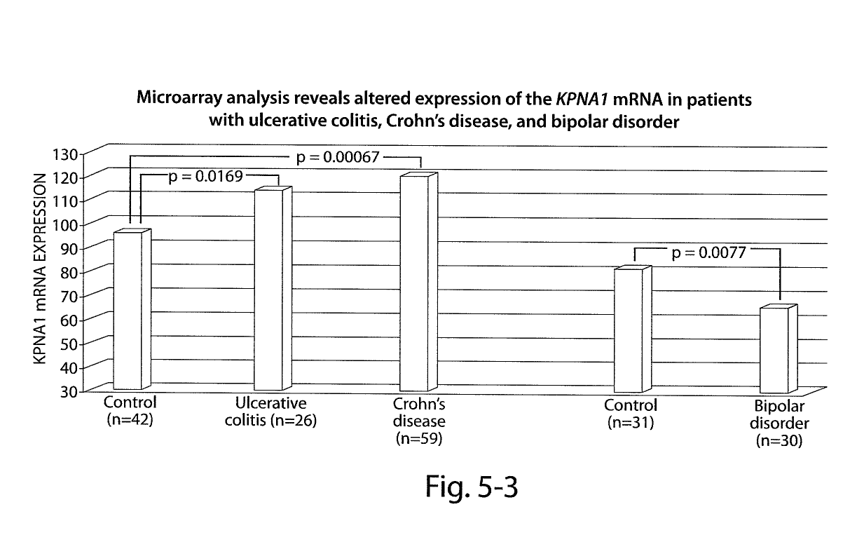

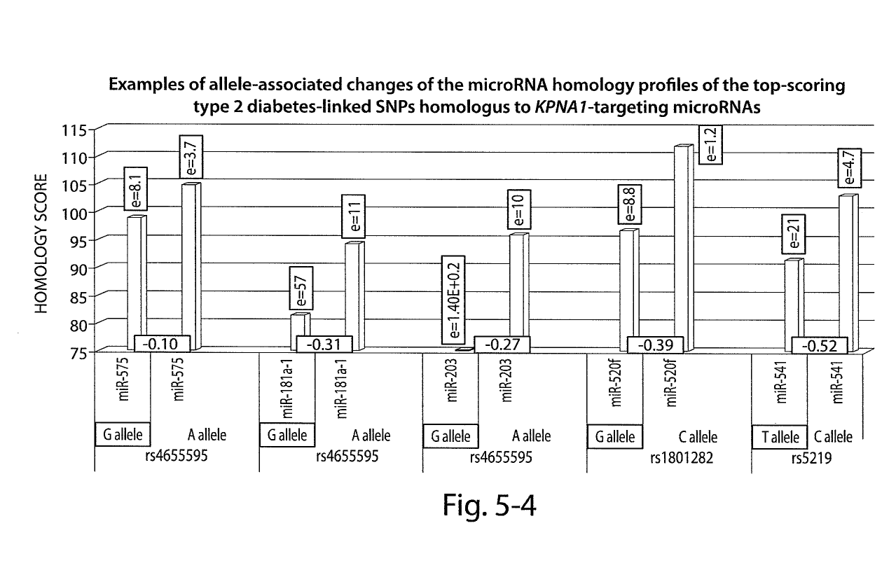

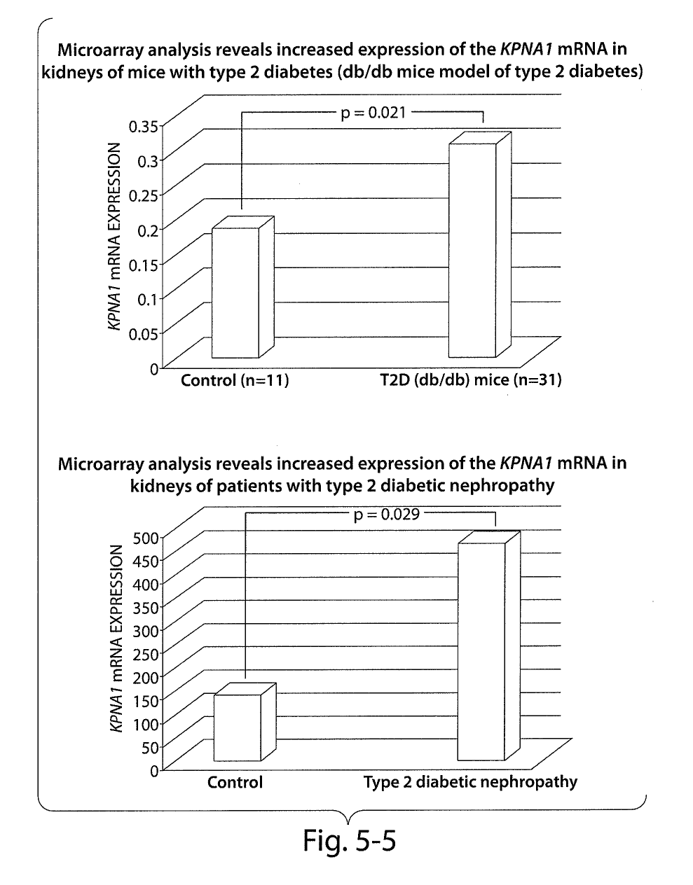

Altered functions of the nuclear import pathway may have a significant contribution to the pathogenesis of many common human disorders. KPNA1 expression was found to be altered in patients diagnosed with many different diseases (see FIG. 5), which can be exploited for diagnostic applications. It would be of interest to determine whether the KPNA1 gene and/or nuclear import pathway are amenable for targeted therapeutic interventions.

To confirm the validity of the findings using disease-linked SNPs identified in separate studies and derived from independent data sets, the sequence homology profiling of 23 SNPs with most significant evidence for associations with type 2 diabetes was carried out. A set of 8 microRNAs which have at least 2 sequence homology counterparts among 12 top-scoring T2D-linked SNPs was identified (Table 3).

TABLE 3
12 SNPs and 8 microRNAs comprising a consensus phenocode of type 2 diabetes (T2D)

| microRANs/ | | | | | | | | | |
|---|---|---|---|---|---|---|---|---|---|
| SNPs | miR-548 | mir-let-7 | mir-1294 | miR-518 | miR-520 | miR-526 | miR-556 | miR-573 | P value, SNP |
| rs13071168 | | 1 | 1 | | | | | | 1.47823E−08 |
| rs1801282 | | | | 1 | 1 | 1 | | | 5.9131E−13 |
| rs4402960 | | 1 | | | | | | | 0.00019197 |
| rs6931514 | | | | | | | 1 | | 0.00019197 |
| rs10282940 | | 1 | | | | | | 1 | 1.47823E−08 |
| rs7020996 | 1 | | | | | | | | 0.00019197 |
| rs7903146 | | | | 1 | 1 | 1 | | | 5.9131E−13 |
| rs8050136 | 1 | | | | | | | | 0.00019197 |
| rs17705177 | 1 | | 1 | | | | | | 1.47823E−08 |
| rs864745 | 1 | | | | | | | | 0.00019197 |
| rs7578597 | | | | | | | 1 | | 0.00019197 |
| rs9472138 | | | | | | | | 1 | 0.00019197 |
| Score | 4 | 3 | 2 | 2 | 2 | 2 | 2 | 2 | |
| P value, mIR | 3.74E−06 | 0.000221 | 0.00732 | 0.00732 | 0.00732 | 0.00732 | 0.00732 | 0.00732 | |

Legend:
Abbreviations:
T2D, type 2 diabetes.
Numbers [1] in the table indicate the SNPs with sequence homology to corresponding microRNAs. Bold blue color highlights microRNAs with target potentials toward mRNAs encoded by the KPNA1 gene. Lists of predicted mRNA targets were identified using the TargetScan database (http://www.targetscan.org). The score values represent the total number of SNPs with sequence honology to a given microRNA. 500000 SNPs were analyzed for associations with T2D to identify 23 SNPs with most significant associations shown in the Table 2 and Table S3(3). Sequence homology profiling of 23 SNPs identified 12 SNPs with multiple call events of sequence homology to at least 2 microRNAs which are listed in the Table 3. To estimate the likelihood of the occurrence of multiple homology call events by chance, we carried out the hypergeometric distribution test and calculated the corresponding P values.

As with the SNPs and microRNAs comprising the consensus phenocode of human diseases shown in Table 1, five of eight T2D-associated microRNAs have the potential to target KPNA1 mRNAs and 10 of 12 SNPs listed in the Table 3 exhibit sequence homology to microRNAs which are predicted to target KPNA1 gene-encoded mRNAs. These results indicate that the proposed approach is broadly applicable for molecular and genetic definitions of disease-specific phenocodes based on analysis of the sequence homology-driven associations of disease-linked SNPs, microRNAs, and mRNA target genes.

Proof of principle validation of this integrative genomics-based approach to identification of a phenocode of human diseases revealing sequence homology-driven associations between disease-linked SNPs, microRNAs, and mRNA targets was carried out. This approach could be utilized for systematic identification and analysis of disease-specific phenocodes and test the practical utility of this strategy.

Sequence homology profiling of the allelic sequences of the 81 SNP loci located at distinct chromosomal regions of human genome and manifesting most significant associations with seven common human diseases was performed as shown in Example 1, infra.

### A SNP-Guided MicroRNA Map of Fifteen Common Human Disorders Identifies a Consensus Disease Phenocode Aiming at Principal Components of the Nuclear Import Pathway

As noted, recent large-scale genome-wide association (GWA) studies of SNP variations captured many thousands individual genetic profiles of H. sapiens and have facilitated identification of significant genetic traits which are highly likely to influence the pathogenesis of several major human diseases. Integrative genomics principles were applied to interrogate relationships between structural features and gene expression patterns of disease-linked SNPs, microRNAs, and mRNAs of protein-coding genes in association to phenotypes of 15 major human disorders, namely bipolar disease (BD); rheumatoid arthritis (RA); coronary artery disease (CAD); Crohn's disease (CD); type 1 diabetes (T1D); type 2 diabetes (T2D); hypertension (HT); ankylosing spondylitis (AS); Graves' disease (autoimmune thyroid disease; AITD); multiple sclerosis (MS); breast cancer (BC); prostate cancer (PC); systemic lupus erythematosus (SLE); vitiligo-associated multiple autoimmune disease (VIT); and ulcerative colitis (UC). A set of 250 SNPs, which were unequivocally associated with common human disorders based on multiple independent studies of 220,124 individual samples comprising 85,077 disease cases and 129,506 controls were selected for sequence homology profiling. The analysis revealed a systematic primary sequence homology/complementarity-driven pattern of associations between disease-linked SNPs, microRNAs, and protein-coding mRNAs defined here as a human disease phenocode.

This approach was utilized to draw SNP-guided microRNA maps of major human diseases and define a consensus disease phenocode for fifteen major human disorders. A consensus disease phenocode comprises 72 SNPs and 18 microRNAs with an apparent propensity to target mRNA sequences derived from a single protein-coding gene, KPNA1. Each of microRNAs in this elite set appears linked to at least three common human diseases and has potential protein-coding mRNA targets among the principal components of the nuclear import pathway. The validity of these findings was confirmed by analyzing independent sets of most significant disease-linked SNPs and demonstrating statistically significant KPNA1-gene expression phenotypes associated with human genotypes of CD, BD, T2D, and RA populations. Variations in DNA sequences associated with multiple human diseases may affect phenotypes in trans via non-protein-coding RNA intermediaries interfering with functions of microRNAs and defines the nuclear import pathway as a potential major target in 15 common human disorders.

Sequence Homology Profiling of Disease-Linked SNPs Identifies the MicroRNA Map of Common Human Disorders

The sequence homology profiling was carried out of 93 SNPs which are most significantly associated with seven common human disorders, namely bipolar disease (BD); rheumatoid arthritis (RA); coronary artery disease (CAD); Crohn's disease (CD); type 1 diabetes (T1D); type 2 diabetes (T2D); and hypertension (HT). It was found that 77 of 93 SNP sequences (83%) manifest homology or complementarity to 153 human microRNAs exceeding the default level of statistical threshold for the e-value of 10). Interestingly, a majority of SNPs (12 of 16; 75%) with no detectable homology to human microRNAs at the default level of significance was derived from the SNPs with moderate disease association levels (see Table 4), suggesting that SNPs with the strongest disease association are enriched for sequences homologous to the microRNAs.

TABLE 4
29 SNPs and 10 microRNAs comprising a consensus phenocode of six common human
diseases

| | SNP | microRNAI | mir- | | | mir- | | | | | | mir- | P value, |
|---|---|---|---|---|---|---|---|---|---|---|---|---|---|
| Disease | Chromosome | reported SNP | 125 | mill.181 | miR.29 | 519 | miR.541 | mill-147 | mir.199 | miR-297 | mir.520 | 558 | SNP |
| BD | 16p12 | rs420259 | | | 1 | | | | | | | | 0.00058 |
| BD | 9q32 | rs10982256 | | | | | | | | 1 | | | 0.00058 |
| BD | 14832 | rs11622475 | | | | | | | 1 | | | | 0.00058 |
| BD | 8p12 | rs2609653 | | | | | | 1 | | | | | 0.00058 |
| BD | 6p21 | m6458307 | | | 1 | | | | | | | | 0.00058 |
| CAD | 9p21 | rs1333049 | | | | 1 | | | | | 1 | | 1.5E−07 |
| CAD | 5821 | rs383830 | | | | | | | | | | 1 | 0.00058 |
| CD | 5q33 | rs1000113 | | | | | | | | | | 1 | 0.00058 |
| CD | 1p31.3 | 1111209026 | 1 | | | | | | 1 | | | | 1.5E−07 |
| CD | 1p31 | rs11805303 | | | | 1 | | | | | | | 0.00058 |
| CD | 5p13 | rs17234657 | | | | | | 1 | | | | | 0.00058 |
| CD/T1D | 18211 | rs2542151 | 1 | | | | | | | | | | 0.00058 |
| CD | 6p22 | rs8908425 | | 1 | | | | | | | | | 0.00058 |
| CD | 7q36 | rs7807268 | 1 | | | | | | | | | | 0.00058 |
| CD | 6p21 | rs9469220 | | 1 | | | | | | | | | 0.00058 |
| RA/T1D | 1p13.3- | rs2476601 | | | | | 1 | | | | | | 0.00058 |
| RA | 6p2I.3 | rs615672 | | | | | 1 | | | | | | 0.00058 |
| RA | 6 MHC | rs6457617 | | | | | | | | 1 | | | 0.00058 |
| RA | 1p36 | rs6684865 | | 1 | | | | | | | | | 0.00058 |
| RA | 6q23 | rs6920220 | | | | 1 | | | | | | | 0.00058 |
| RA | 13q12 | rs9550642 | | | | | | | 1 | | | | 0.00058 |
| T1D | 12p13 | r83764021 | | | | | | 1 | | | | | 0.00058 |
| T1D | 2q24 | rs3788964 | | | 1 | | | | | | | | 0.00058 |
| T1D | 1203 | rs11052552 | | | | | | | | 1 | | 1 | 1.5E−07 |
| T1D | 1q42 | m2639703 | | | 1 | | | | | | | | 0.00058 |
| T2D | 3p25 | rs1801282 | | | | 1 | | | | | 1 | | 1.5E−07 |
| 120 | 1031 | rs4655595 | | 1 | | | | | | | | | 0.00058 |
| T2D | 11015.1 | rs5219 | | | | | 1 | | | | | | 0.00058 |
| T2D | 10q25.3 | rs7903146 | | | | | | | | | 1 | | 0.00058 |
| | | SCORE | 3 | 4 | 4 | 4 | 3 | 3 | 3 | 3 | 3 | 3 | |
| | | P value, | 0.00 | 0.00 | 0000 | 0.00 | 0.006 | 0.006 | 0006 | 0.006 | 0.00 | 0.00 | |

BD, bipolar disease;
CAD, coronary artery disease;
CD, Crohn's disease;
RA, rheumatoid arthritis;
T1D, type 1 diabetes;
T2D, type 2 diabetes.
Numbers [1] in the table indicate the SNPs with sequence homology to corresponding microRNAs. The score values represent the total number of SNPs with sequence homology to a given microRNA. 500000 SNPs were analyzed for associations with common human diseases to identify 93 SNPs with most significant associations. Sequence homology profiling of 93 SNPs identified 29 SNPs with multiple call events of sequence homology to elite set of 10 microRNAs which are listed in the Table 4. To estimate the likelihood of the occurrence of multiple homology call events by chance, we carried out the hypergeometric distribution test and calculated the corresponding p values.

Consistently, 90% of SNPs (34 of 38) with the most significant disease associations manifest sequence homology to human microRNAs compared to 78% of SNPs (43 of 55) with the moderate disease association levels.

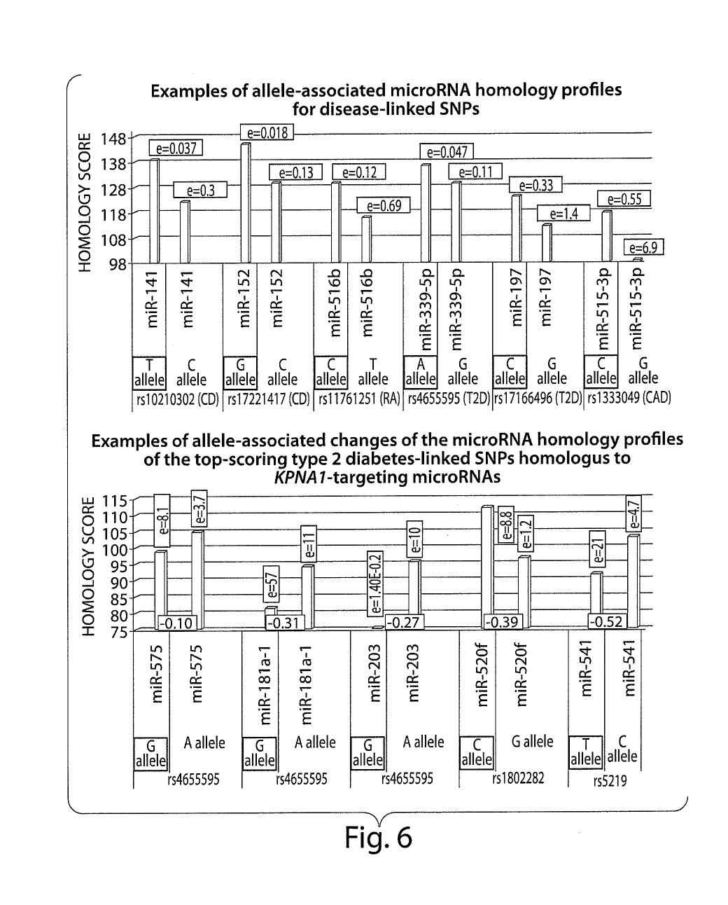

In many instances, the profiles of sequence homology interactions between SNPs and microRNAs manifest distinct allele-specific patterns (see FIG. 6), which is consistent with the postulated regulatory and/or disease-causing functions of these sequences. An elite set of 10 microRNAs which have at least 3 sequence homology counterparts among 29 top-scoring disease-associated SNPs was identified as shown in Table 4. Moreover, the probability that multiple sequence homology calls occurred by chance was estimated and found that it is highly unlikely. This sequence homology-driven associations of disease-linked SNP and microRNAs is designated herein as a SNP-guided microRNA map (“MirMap”) of human diseases (Table 4). Taken together, these data support the hypothesis that allele-associated differences in SNP sequence homology to microRNAs may be causally linked to disease phenotypes. Accordingly, in all examples shown in FIG. 6, higher microRNA-targeting potency of the risk alleles is postulated.

A Consensus MicroRNA Map of Human Disorders Points to mRNA Targets Derived from the Single Protein-Coding Gene, KPNA1

Next, it was determined whether identified set of 10 microRNAs would have the potential to target a common group of mRNAs. The lists of predicted mRNA targets were retrieved for each of the 10 microRNA shown in Table 4 using TargetScan database and searched for concordant sets of mRNA targets based on the context scores. Remarkably, the analysis reveals that 70% of identified microRNAs (Table 4) have the potential to target mRNA sequences derived from a single protein-coding gene, namely KPNA1 (importin alpha 5; Table 5).

TABLE 5
Majority of the consensus phenocode microRNAs have targeting potentials toward
mRNAs encoded by the importin alpha 5 (KPNAJ) gene

| Targeted | | | | | | | | | |
|---|---|---|---|---|---|---|---|---|---|
| importins/ | | | | | | | | | |
| microRNAs | KPNA1 | KPNA2 | KPNA3 | KPNA4 | KPNA5 | KPNA6 | KPNB1 | KPNB2 | KNPB3 |
| mir-125 | | | | | | mir-125 | mir-125 | | |
| miR-181 | miR-181 | | | miR-181 | | | miR-181 | miR-181 | miR-181 |
| miR-29 | miR-29 | | | miR-29 | | | miR-29 | | |
| mir-519 | mir-519 | | mir-519 | | | | | | mir-519 |
| miR-541 | miR-541 | | | | | | | | |
| miR-147 | miR-147 | | | | | miR-147 | miR-147 | | miR-147 |
| mir-199 | | | | mir-199 | | mir-199 | mir-199 | | |
| miR-297 | | | | | | | | | |
| mir-520 | mir-520 | mir-520 | mir-520 | | | mir-520 | mir-520 | mir-520 | mir-520 |
| mir-558 | mir-558 | | | | | | | | mir-558 |
| Score | 7 | 1 | 2 | 3 | 0 | 4 | 6 | 2 | 5 |
| P value | 0.006034 | 0.302798 | 0.284126 | 0.127604 | 0.68583 | 0.181507 | 0.000137 | 0.286379 | 0.077955 |

Human importin-targeting microRNAs were identified using TargetScan database. p values were calculated using hyper-geometric distribution tests. They represent the estimates of the likelihood of obtaining score values by chance and take into account the numbers of all screened for homology microRNAs and the number of microRNAs which are predicted to target a given importin gene.

Moreover, 22 of 29 SNPs listed in the Table 4 manifest sequence homology to microRNAs which are predicted to target KPNA1 gene (Table 4), thereby indicating that sequence homology to the KPNA1-targeting microRNAs is a common structural (and, perhaps, functional) feature of many SNPs associated with multiple major human diseases. To test whether KPNA1-targeting is specific, it was estimated the predicted targeting effect by the consensus microRNAs on distinct set of mRNAs which are derived from five other importin-encoding genes and are functionally and structurally closely related to the KPNA1 gene. The predicted targeting effect on mRNAs of five distinct importins was found not to reach the threshold of statistical significance to exclude the likelihood of occurrence of multiple calls by chance (see Table 5). Thus, KPNA1 mRNA targeting by the consensus microRNAs is specific. It is tempting to speculate that KPNA1 is the gene representing a common disease target in at least six major human disorders (BD; RA; CAD; CD; T1D; T2D). To define the sequence homology-driven associations of disease-linked SNPs, microRNAs, and mRNA target genes as a consensus phenocode of human diseases was proposed.

A Consensus microRNA Map of Type 2 Diabetes (T2D) Identifies KPNA1 mRNA Targets

To confirm validity of these findings using disease-linked SNPs identified in separate studies and derived from independent data sets, the sequence homology profiling of 23 SNPs with most significant evidence for associations with type 2 diabetes was performed. The analysis identifies a set of 8 microRNAs which have at least 2 sequence homology counterparts among 12 top-scoring T2D-linked SNPs (Table 6).

TABLE 6
12 SNPs and 8 microRNAs comprising a consensus phenocode of type 2
diabetes (T2D)

| microRNAs/ | miR- | | mir- | | miR- | miR- | miR- | miR- | P value, |
|---|---|---|---|---|---|---|---|---|---|
| SNPs | 548 | mir-let-7 | 1294 | miR-518 | 520 | 526 | 556 | 573 | SNP |
| rs13071168 | | 1 | 1 | | | | | | 1.47823E−08 |
| rs1801282 | | | | 1 | 1 | 1 | | | 5.9131E−13 |
| rs4402960 | | 1 | | | | | | | 0.00019197 |
| rs6931514 | | | | | | | 1 | | 0.00019197 |
| rs10282940 | | 1 | | | | | | 1 | 1.47823E−08 |
| rs7020996 | 1 | | | | | | | | 0.00019197 |
| rs7903146 | | | | 1 | 1 | 1 | | | 5.9131E−13 |
| rs8050136 | 1 | | | | | | | | 0.00019197 |
| rs17705177 | 1 | | 1 | | | | | | 1.47823E−08 |
| rs864745 | 1 | | | | | | | | 0.00019197 |
| rs7578597 | | | | | | | 1 | | 0.00019197 |
| rs9472138 | | | | | | | | 1 | 0.00019197 |
| Score | 4 | 3 | 2 | 2 | 2 | 2 | 2 | 2 | |
| P value, miR | 3.74E−06 | 0.000221 | 0.00732 | 0.00732 | 0.00732 | 0.00732 | 0.00732 | 0.00732 | |

T2D, type 2 diabetes.
Numbers [1] in the table indicate the SNPs with sequence homology to corresponding microRNAs. Bold color highlights microRNAs with target potentials toward mRNAs encoded by the KPNAI gene. Lists of predicted mRNA targets were identified using the TargetScan database. The score values represent the total number of SNPs with sequence homology to a given microRNA. 500,000 SNPs were analyzed for associations with T2D to identify 23 SNPs with most significant associations shown in the Table 2 and Table 53. Sequence homology profiling of 23 SNPs identified 12 SNPs with multiple call events of sequence homology to the elite set of 8 microRNAs which are listed in the Table 3. To estimate the likelihood of the occurrence of multiple homology call events by chance, the hypergeometric distribution test was carried out and the corresponding p values were calculated.

Similar to the SNPs and microRNAs comprising the consensus phenocode of human diseases as shown in Table 4, five of eight T2D-associated microRNAs have the potential to target KPNA1 mRNAs and 10 of 12 SNPs listed in the Table 6 exhibit sequence homology to microRNAs which are predicted to target KPNA1 gene-encoded mRNAs. (see Tables 6 & 7).

TABLE 7
Majority of the consensus T2D phenocode microRNAs have targeting potentials
toward mRNAs encoded by the importin alpha 5 (KPNA1) gene

| Targeted | | | | | | | | | |
|---|---|---|---|---|---|---|---|---|---|
| importins/ | | | | | | | | | |
| microRNAs | KPNA1 | KPNA2 | KPNA3 | KPNA4 | KPNA5 | KPNA6 | KPNB1 | KPNB2 | KPNB3 |
| miR-548 | miR-548 | | miR-548 | miR-548 | miR-548 | miR-548 | miR-548 | miR-548 | miR-548 |
| mir-let-7 | mir-let-7 | | | mir-let-7 | | mir-let-7 | | | mir-let-7 |
| mir-1294 | | | | | | | | | |
| miR-518 | | | | | | miR-518 | | | |
| miR-520 | miR-520 | miR-520 | miR-520 | | | miR-520 | miR-520 | miR-520 | miR-520 |
| miR-526 | miR-526 | | | | | miR-526 | | | |
| miR-556 | | | | | | | | miR-556 | |
| miR-573 | miR-573 | | | | | miR-573 | miR-573 | miR-573 | |
| Score | 5 | 1 | 2 | 3 | 1 | 6 | 3 | 4 | 3 |
| P value | 0.018957 | 0.243083 | 0.219574 | 0.208116 | 0.207207 | 0.002355 | 0.02418 | 0.054066 | 0.104329 |

Human importin-targeting microRNAs were identified using Target Scan database. P values were calculated using hypergeometric distribution tests. They represent the estimates of likelihood of obtaining score values by chance and take into account the numbers of all screened for homology microRNAs and the number of microRNAs which are predicted to target a given importin gene.

These results indicate that this approach is broadly applicable for molecular and genetic definitions of disease-specific phenocodes based on analysis of the sequence homology-driven associations of disease-linked SNPs, microRNAs, and mRNA target genes.

Microarray Analysis Reveals KPNA1 Gene Expression Phenotypes Associated with Human Genotypes of CD, BD, RA, and T2D Populations

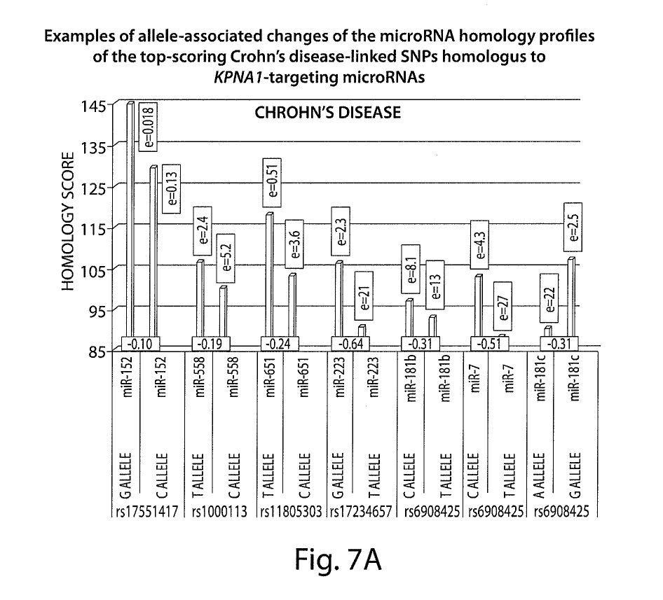

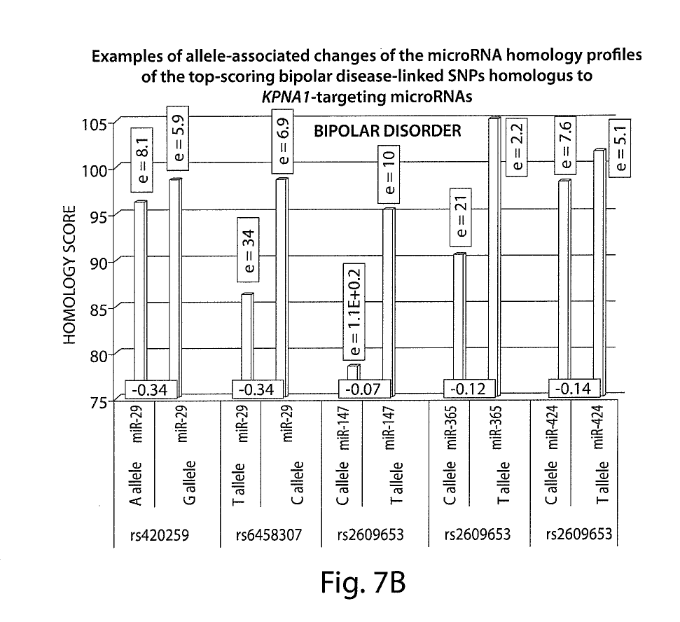

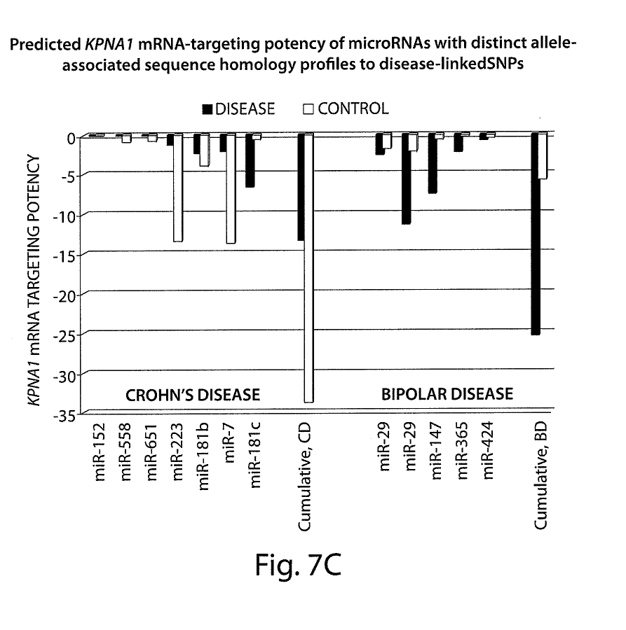

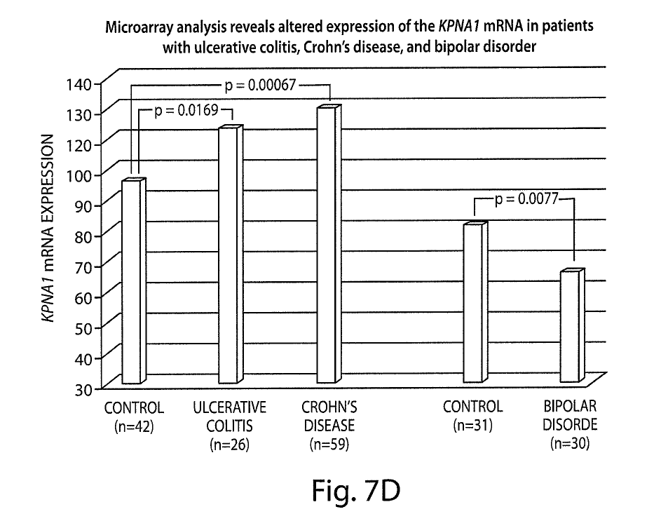

According to the disease phenocode hypothesis, higher homology to KPNA1-targeting microRNAs of the multiple risk alleles in patients with Crohn's disease (CD) is predicted to have a cumulative increased microRNA-interference effect which would diminish a cumulative KPNA1 mRNA-targeting potency of microRNAs. Correspondingly, KPNA1 gene expression analysis demonstrates that the human CD genotype is associated with increased KPNA1 mRNA expression levels (see FIG. 7A, C, D). In contrast, lower SNP/microRNA sequence homology-driven decreased microRNA-interference potential of the multiple risk alleles in patients with bipolar disorder (BD) is predicted to increase a cumulative KPNA1 mRNA-targeting potency of multiple microRNAs. Accordingly, gene expression analysis experiments reveal decreased KPNA1 mRNA expression levels in BD patients (see FIG. 7B, C, D). Thus, it would be of interest to determine whether the KPNA1 gene and/or nuclear import pathway are amenable for targeted therapeutic interventions.

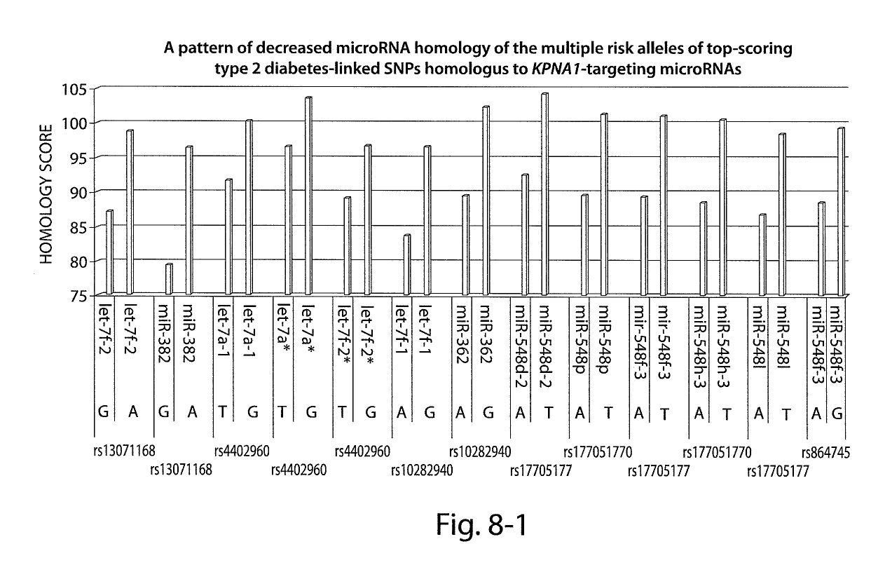

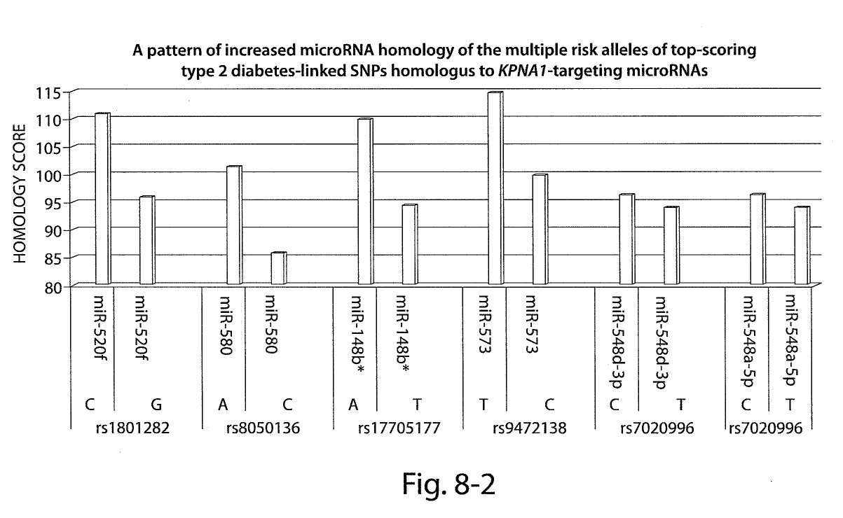

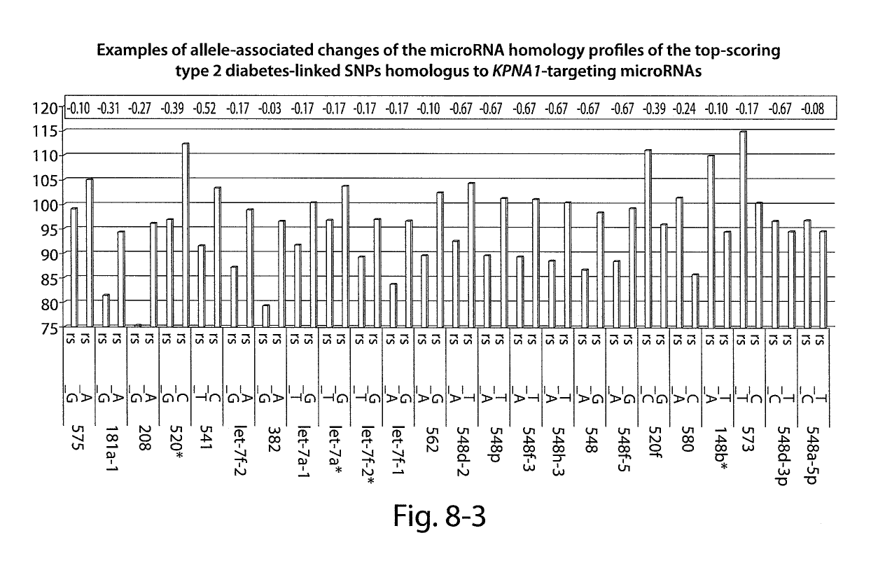

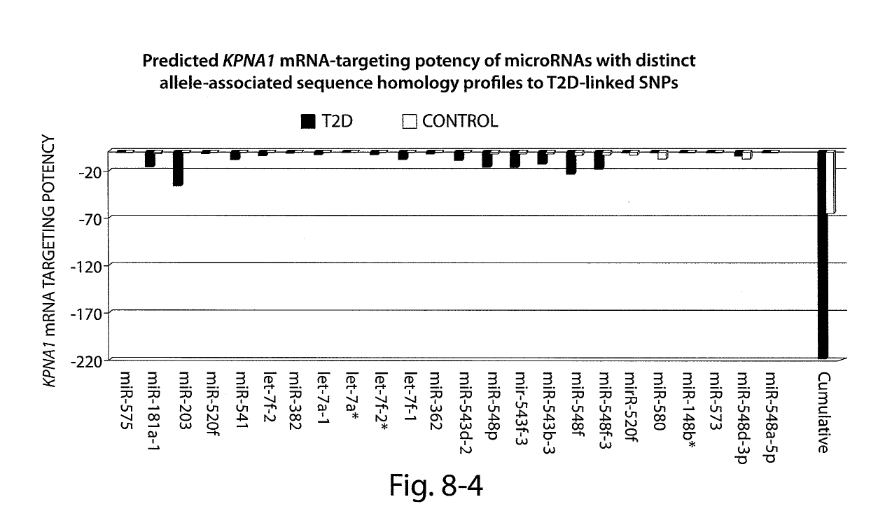

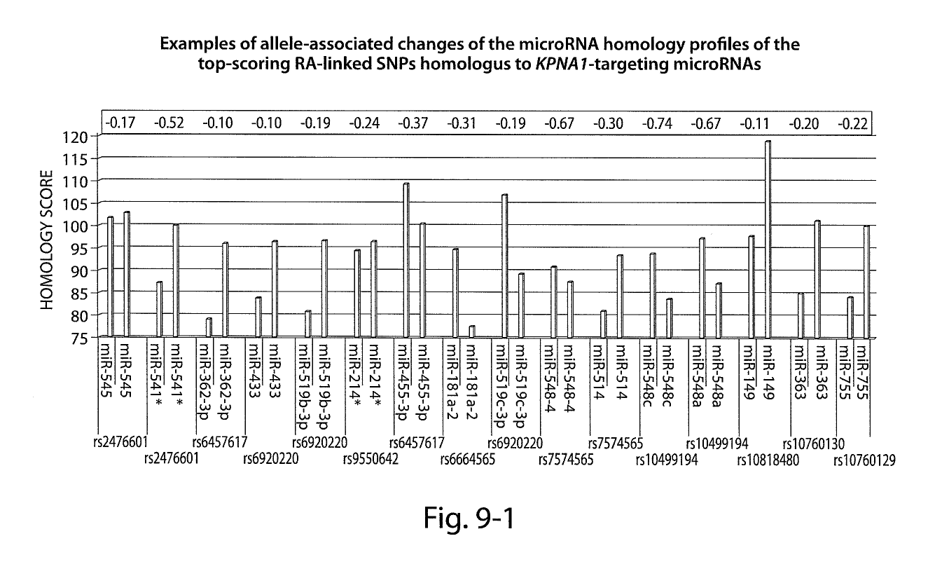

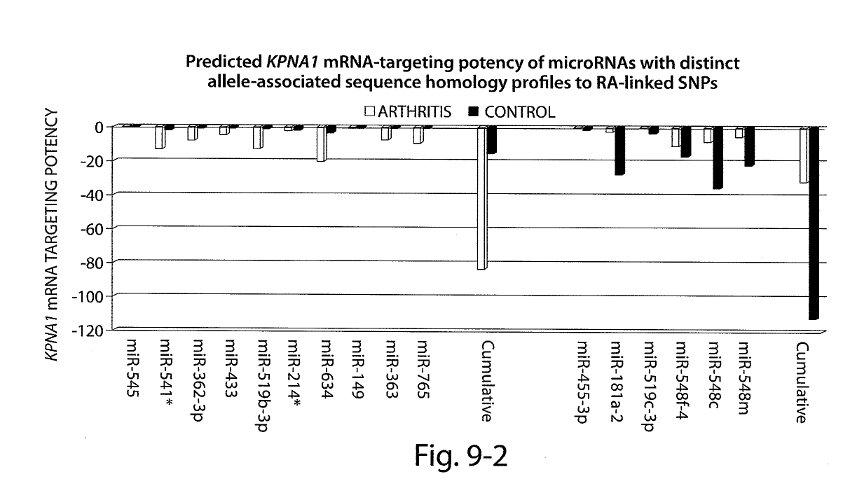

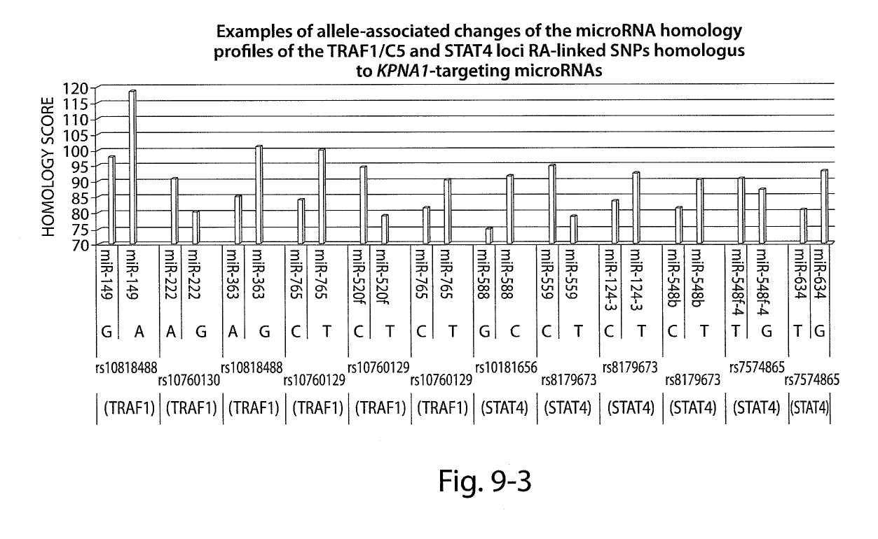

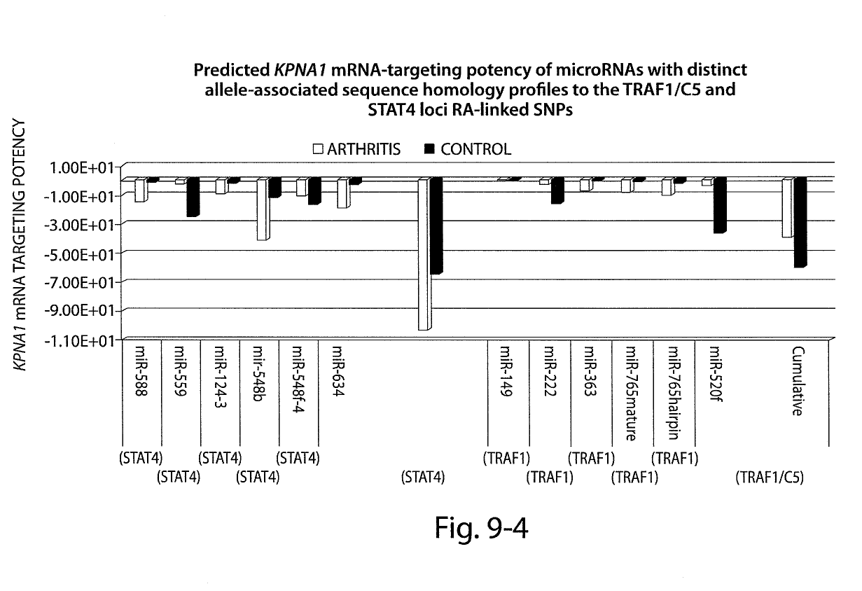

Detailed analysis of the sequence homology profiles of the T2D-linked SNPs reveals two distinct patterns of changes of microRNA-targeting potentials associated with risk alleles (see FIG. 8), thereby suggesting that individual patients may carry highly diverse spectrum of changes in the KPNA1 mRNA expression driven by the unique balance of disease-causing alleles. These observations were verified using independent sets of 16 disease-linked SNPs identified in recent high-powered GWA studies of RA patients which unequivocally confirmed five RA susceptibility genes (HLA-DRB1, PTPN22, OLIG3/TNFAIP3, STAT4 and the TRAF1/C5). As with the T2D patients, the sequence homology profiles of the RA-linked SNPs identify two distinct patterns of changes of microRNA-targeting potentials associated with risk alleles (see FIG. 9).

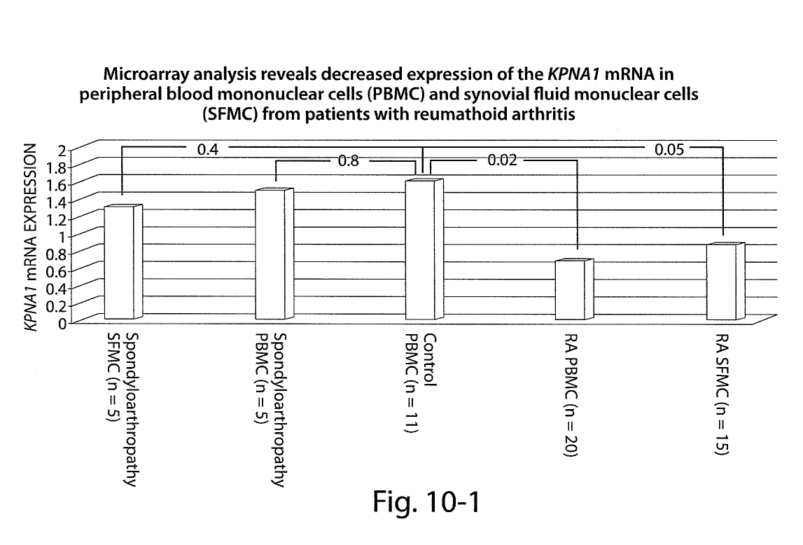

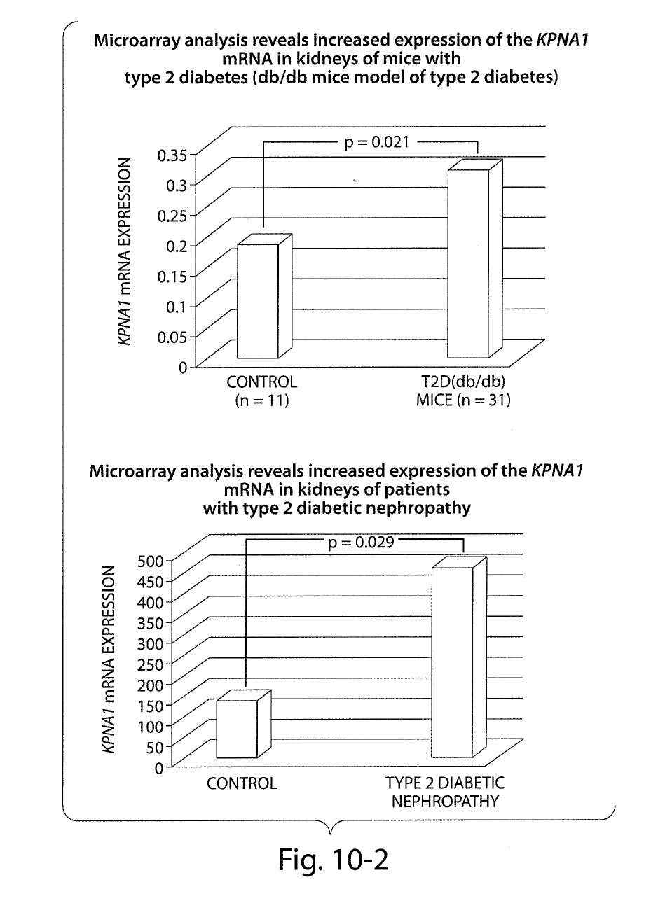

In both T2D and RA patients, the pattern of decreased sequence homology scores of disease-linked SNPs to KPNA1-targeting microRNAs is predicted to facilitate an intracellular context favoring higher KPNA1-targeting potency by multiple microRNAs thus increasing the probability of the KPNA1-deficient phenotypes. (See FIGS. 8 & 9). Conversely, the pattern of increased sequence homology scores of disease-linked SNPs to KPNA1-targeting microRNAs is predicted to facilitate an intracellular context favoring lower KPNA1-targeting potency by multiple microRNAs thus increasing the probability of the KPNA1-overexpression phenotypes (see FIGS. 8 & 9). Interestingly, in RA patients, distinct patterns of changes of the predicted KPNA1 mRNA-targeting potency of disease-linked microRNAs appear to segregate with distinct genetic loci (see FIG. 9), adding further support to the idea of the highly diverse spectrum of the KPNA1 phenotypes which is expected in individual patients within disease-susceptible populations. The direction and amplitude of the KPNA1 mRNA expression changes would depend on the unique balance of the disease-causing SNPs in each patient as well as on tissue-specificity of microRNA and SNP-encoded RNA expression, which would dictate the necessity of highly individualized therapeutic approaches tailored to individual phenotypes. Gene expression analysis identifies distinct KPNA1 phenotypes in T2D and RA patients. (See FIG. 10). Microarray analysis reveals that KPNA1 mRNA expression level is decreased in peripheral blood mononuclear cells (PMBC) and synovial fluid mononuclear cells (SFMC) from RA patients, whereas in kidneys of T2D patients with diabetic nephropathy and db/db mice the expression of KPNA1 mRNA is elevated. (See FIG. 10).

SNP-Guided microRNA Maps of Multiple Human Disorders Reveal a Consensus Disease Phenocode for 15 Common Human Diseases

To explore the utility of the disease phenocode concept, multiple independent sets of SNPs manifesting strong associations with additional seven common human disorders namely ankylosing spondylitis (AS); autoimmune thyroid disease (AITD); multiple sclerosis (MS); breast cancer (BC); prostate cancer (PC); systemic lupus erythematosus (SLE); and ulcerative colitis (UC) were analyzed. To build the SNP-guided microRNA maps of individual human disorders, sequence homology profiling of 18 AITD-linked SNPs; 15 MS-linked SNPs; 12 SNPs associated with autoimmune disorders (AID); 20 AS-linked SNPs; 16 BC-linked SNPs; 18-SLE-linked SNPs; 18 PC-linked SNPs; 18 vitiligo-associated multiple autoimmune disease SNPs (VIT) and 5 UC-linked SNPs which were identified recently in high-power association studies were carried out. Analysis of individual SNP-guided microRNA maps of human diseases confirmed in all instances the apparent propensity to target the KPNA1-encoded mRNAs by the disease-linked microRNAs, suggesting that nuclear import pathway may represent a critically important target in multiple major human disorders. Combination of the analytical power of SNP-guided microRNA maps of 14 human diseases reveals a consensus disease phenocode comprising 65 SNPs and 17 microRNAs. (See Table 8).

TABLE 8
72 SNPs and 18 microRNAs comprising a consensus phenocode of 15 common human disorders

| microRNA/ | miR | | mir- | miR- | | | miR- | miR- | miR- | miR- |
|---|---|---|---|---|---|---|---|---|---|---|
| SNP | 125 | let-7 | 519 | 520 | mir-143 | miR-181 | 541 | 548 | 1238 | 147 |
| ATM-333 | | | | MS | | | | | | |
| rs1000113 | | | | | | | | | | |
| rs10282940 | | T2D | | | | | | | | |
| rs10516487 | | SLE | | | | | | | | |
| rs10982256 | | | | | | | | | | |
| rs11052552 | | | | | | | | | | |
| rs11209026 | CD | | | | | | | | | |
| rs11554159 | | | | | AS/MS | | | | | |
| rs11574422 | | | | | | | | | | |
| rs11574637 | SLE | | | | | | | | | |
| rs11622475 | | | | | | | | | | |
| rs11805303 | | | CD | | | | | | | |
| rs12067507 | | | | | | | | AID | AID | |
| rs12143301 | | AS | | | | | | | | |
| rs13071168 | | T2D | | | | | | | | |
| rs1333049 | | | CAD | CAD | | | | | | |
| rs17234657 | | | | | | | | | | CD |
| rs17266594 | | | | | | | | | | |
| rs1729674 | | | | | | | | AID | | |
| rs17705177 | | | | | | | | T2D | | |
| rs1801282 | | | T2D | T2D | | | | | | |
| rs1859962 | PC | | | | | | | | | |
| rs2229358 | | | | | | | | | | |
| rs2232337 | | | | | | | | AID | | |
| rs2302250 | | | | | AS | | | | | |
| rs2303759 | | | | | | | | | | |
| rs2476601 | | | | | | | RA/T1D | | | |
| rs2542151 | CD/T1D | | | | | | | | | |
| rs2609653 | | | | | | | | | | BD |
| rs27044 | | | | | | | | | AS | |
| rs30187 | | | | AS | | | | | | |
| rs3197999 | | AS/UC | | | | | | | | |
| rs34536443 | | | | | | | | | | |
| rs35285785 | | | | | | | | | AITD | |
| rs3760511 | PC | PC | | | | | | | | |
| rs3764021 | | | | | | | | | | T1D |
| rs3788964 | | | | | | | | | | |
| rs383830 | | | | | | | | | | |
| rs4242382 | | PC | | | | | | | | |
| rs4402960 | | T2D | | | | | | | | |
| rs4430796 | | | | | PC | | | | | |
| rs4548893 | | SLE | | | | | | | | |
| rs465595 | | | | | | T2D | | | | |
| rs4986790 | | | | | | | | | | |
| rs5219 | | | | | | | T2D | | | |
| rs615672 | | | | | | | RA | | | |
| rs6457617 | | | | | | | | | | |
| rs6684865 | | | | | | RA | | | | |
| rs6908425 | | | | | | CD | | | | |
| rs6920220 | | | RA | | | | | | | |
| rs6957669 | MS | | | | | | | | | |
| rs7020996 | | | | | | | | T2D | | |
| rs7313899 | | | | | | BC | | | | |
| rs7501939 | | | | | PC | | | | | |
| rs7578199 | | | | | AITD | | | | | |
| rs7807268 | CD | | | | | | | | | |
| rs7837688 | | PC | | | | | | | | |
| rs7903146 | | | | T2D | | | | | | |
| rs7975069 | | | | | | | | | | |
| rs8050136 | | | | | | | | T2D | | |
| rs864745 | | | | | | | | T2D | | |
| rs9469220 | | | | | | CD | | | | |
| rs9550642 | | | | | | | | | | |
| rs9616915 | | | | | | BC | | | | |
| rs983085 | | | | | | | | | | |
| rs9888739 | | | | | | | | SLE | | |
| rs9939768 | AS | | | | | | | | | |
| rs12150220 | | VIT | | | | | | | | |
| rs2733359 | | | | | | | | | | |
| rs7223628 | | | | | | | VIT | | | |
| rs8182354 | | | VIT | VIT | | | | | | |
| rs961826 | | VIT | | | | | | VIT | | |
| SCORE | 6 | 6 | 5 | 5 | 4 | 4 | 4 | 4 | 3 | 3 |
| DISEASES | AS | AS | CAD | AS | AITD | BC | RA | AID | AITD | BD |
| | CD | PC | CD | CAD | AS | CD | RA/T1D | SLE | AS | CD |
| | CD/T1D | SLE | RA | MS | AS/MS | RA | T2D | T2D | AID | T1D |
| | MS | T2D | T2D | T2D | PC | T2D | VIT | VIT | | |
| | PC | UC | VIT | VIT | | | | | | |
| | SLE | VIT | | | | | | | | |
| P value, | 5.9E−07 | 6E−07 | 1.8E−05 | 1.8E−05 | 0.00038 | 0.00038 | 0.000375 | 0.00038 | 0.00543 | 0.00543 |
| microm RNAs | | | | | | | | | | |

| microRNA/ | mir- | mir- | miR- | | mir- | mir- | mir- | mir- | Disease, |
|---|---|---|---|---|---|---|---|---|---|
| SNP | 199 | 200 | 297 | mir-374 | 558 | 662 | 936 | 30 | SNP |
| ATM-333 | | | | | | | | | MS |
| rs1000113 | | | | | CD | | | | CD |
| rs10282940 | | | | | | | | | T2D |
| rs10516487 | | | | | | | | | SLE |
| rs10982256 | | | BD | | | | | | BD |
| rs11052552 | | | T1D | | T1D | | | | T1D |
| rs11209026 | CD | | | | | | | | CD |
| rs11554159 | | | | | | AS/MS | | | AS/MS |
| rs11574422 | | | | | | | AID | | AID |
| rs11574637 | | | | | | | | | SLE |
| rs11622475 | BD | | | | | | | BD | BD |
| rs11805303 | | | | | | | | | CD |
| rs12067507 | | | | | | | | | AID |
| rs12143301 | | | | | | | | | AS |
| rs13071168 | | | | | | | | | T2D |
| rs1333049 | | | | | | | | | CAD |
| rs17234657 | | | | | | | | | CD |
| rs17266594 | | | | SLE | | | | | SLE |
| rs1729674 | | | | | | | | | AID |
| rs17705177 | | | | | | | | | T2D |
| rs1801282 | | | | | | | | | T2D |
| rs1859962 | | | | | | | | | PC |
| rs2229358 | | MS | | | | | | | MS |
| rs2232337 | | | | | | | | | AID |
| rs2302250 | | | | | | | | | AS |
| rs2303759 | | AID | | | | | | | AID |
| rs2476601 | | | | | | | | | RA/T1D |
| rs2542151 | | | | | | | | | CD/T1D |
| rs2609653 | | | | | | | | | BD |
| rs27044 | | | | | | | | | AS |
| rs30187 | | | | | | | | | AS |
| rs3197999 | | | | | | | AS/UC | | AS/UC |
| rs34536443 | | | | | | AID | | | AID |
| rs35285785 | | | | | | | | | AITD |
| rs3760511 | | | | | | | | | PC |
| rs3764021 | | | | | | | | | T1D |
| rs3788964 | | | | | | | | T1D | T1D |
| rs383830 | | | | | CAD | | | | CAD |
| rs4242382 | | | | | | | | | PC |
| rs4402960 | | | | | | | | | T2D |
| rs4430796 | | | | | | | | | PC |
| rs4548893 | | | | | | | | | SLE |
| rs465595 | | | | | | | | | T2D |
| rs4986790 | | | | BC | | | | | BC |
| rs5219 | | | | | | | | | T2D |
| rs615672 | | | | | | | | | RA |
| rs6457617 | | | RA | | | | | | RA |
| rs6684865 | | | | | | | | | RA |
| rs6908425 | | | | | | | | | CD |
| rs6920220 | | | | | | | | | RA |
| rs6957669 | | | | | | | | | MS |
| rs7020996 | | | | | | | | | T2D |
| rs7313899 | | | | BC | | | | | BC |
| rs7501939 | | | | | | | | | PC |
| rs7578199 | | | | | | | | | AITD |
| rs7807268 | | | | | | | | | CD |
| rs7837688 | | | | | | | | | PC |
| rs7903146 | | | | | | | | | T2D |
| rs7975069 | | AITD | | | | | | | T2D |
| rs8050136 | | | | | | | | | T2D |
| rs864745 | | | | | | | | | T2D |
| rs9469220 | | | | | | | | | CD |
| rs9550642 | RA | | | | | | | | RA |
| rs9616915 | | | | | | | | | BC |
| rs983085 | | | | PC | | | | | PC |
| rs9888739 | | | | | | | | | SLE |
| rs9939768 | | | | | | | | | AS |
| rs12150220 | | | | | | | | | VIT |
| rs2733359 | | | | | | | | VIT | VIT |
| rs7223628 | | | | | | | | | VIT |
| rs8182354 | | | | | | | | | VIT |
| rs961826 | | | | | | | | | VIT |
| SCORE | 3 | 3 | 3 | 3 | 3 | 3 | 3 | | |
| DISEASES | BD | AITD | BD | BC | CAD | AS/MS | AS/UC | | |
| | CD | AID | RA | PC | CD | AID | AID | | |
| | RA | MS | T1D | SLE | T1D | | | | |
| P value, | 0.00543 | 0.00543 | 0.00543 | 0.00543 | 0.00543 | 0.00543 | 0.00543 | | |
| microm RNAs | | | | | | | | | |

Score numbers represent the sum of sequence homology profiling-defined associate events of a given microRNA and disease-linked SNPs. Human disorders BD; RA; CAD; CD; T1D; T2D; HT; AS; AITD; MS; BC; PC; SLE; AID; UC. P values were calculated using hypergeometric test.

All 18 microRNAs of this elite consensus set appear linked to multiple common human diseases. Essentially all consensus microRNAs have potential protein-coding mRNA targets among the importin alpha and/or importin beta genes, which were previously defined as the principal functional components of the nuclear import pathway. (See Table 9).

TABLE 9
Importin mRNA-targeting map of the 18 microRNAs comprising a consensus phenocode
of 15 human disorders

| microRNA | KPNA1 | KPNA2 | KPNA3 | KPNA4 | KPNA5 | KPNA6 | KPNB1 | KPNB2 |
|---|---|---|---|---|---|---|---|---|
| miR-125 | | | | | | mir-125 | mir-125 | |
| let-7 | let-7 | | | let-7 | | let-7 | | |
| mir-519 | mir-519 | mir- | mir-519 | mir- | | mir-519d | | |
| | | 519d | | 519d | | | | |
| miR-520 | miR-520 | miR-520 | miR-520 | | | miR-520 | miR-520 | miR-520 |
| mir-143 | mir-143 | | | | | mir-143 | | |
| miR-181 | miR-181 | | | miR- | | | miR-181 | miR-181 |
| | | | | 181 | | | | |
| miR-541 | miR-541 | | | | | | | |
| miR-548 | miR-548 | | miR-548 | miR- | miR- | miR-548 | miR-548 | miR-548 |
| | | | | 548 | 548 | | | |
| miR-1238 | | | | | | | | |
| miR-147 | miR-147 | | | | | miR-147 | miR-147 | |
| mir-199 | | | | mir- | | mir-199 | mir-199 | |
| | | | | 199 | | | | |
| mir-200 | | | mir-200 | mir- | | mir-200 | | mir-200 |
| | | | | 200 | | | | |
| miR-297 | | | | | | | | |
| mir-374 | mir-374 | | | | | mir-374 | | mir-374 |
| mir-558 | mir-558 | | | | | | | |
| mir-662 | | | | | | mir-662 | | |
| mir-936 | | | | | | | | |
| mir-30 | | mir-30 | mir-30 | | | mir-30 | mir-30 | |
| Score, | 10 | 2 | 5 | 5 | 1 | 11 | 7 | 5 |
| microRNA | | | | | | | | |
| P values | 0.009253 | 0.151896 | 0.086628 | 0.07588 | 0.35627 | 0.002435228 | 0.000992 | 0.198872 |

| microRNA | KNPB3 | SCORE | DISEASES | | | | | |
|---|---|---|---|---|---|---|---|---|
| miR-125 | | 6 | AS | CD | CD/T1D | MS | PC | SLE |
| let-7 | let-7 | 6 | AS | PC | SLE | T2D | UC | VIT |
| mir-519 | mir- | 5 | CAD | CD | RA | T2D | VIT | |
| | 519d | | | | | | | |
| miR-520 | miR-520 | 5 | AS | CAD | MS | T2D | VIT | |
| mir-143 | | 4 | AITD | AS | AS/MS | PC | | |
| miR-181 | miR-181 | 4 | BC | CD | RA | T2D | | |
| miR-541 | | 4 | RA | RA/T1D | T2D | VIT | | |
| miR-548 | miR-548 | 4 | AID | SLE | T2D | VIT | | |
| miR-1238 | | 3 | AITD | AS | AID | | | |
| miR-147 | miR-147 | 3 | BD | CD | T1D | | | |
| mir-199 | | 3 | BD | CD | RA | | | |
| mir-200 | mir-200 | 3 | AITD | AID | MS | | | |
| miR-297 | | 3 | BD | RA | T1D | | | |
| mir-374 | mir-374 | 3 | BC | PC | SLE | | | |
| mir-558 | mir-558 | 3 | CAD | CD | T1D | | | |
| mir-662 | | 3 | AS/MS | AID | | | | |
| mir-936 | | 3 | AS/UC | AID | | | | |
| mir-30 | mir-30 | 3 | BD | T1D | VIT | | | |
| Score, | 9 | | | | | | | |
| microRNA | | | | | | | | |
| P values | 0.002175 | | | | | | | |

microRNA scores represent the number of microRNAs with potential to target mRNAs encoded by a given importin gene. Disease score numbers represent the sum of sequence homology profiling-defined association events of a given microRNA and disease-linked SNPs.
Human disorders:
bipolar disease (BD);
rheumatoid arthritis (RA);
coronary artery disease (CAD);
Crohn's disease (CD);
type 1 diabetes (T1D);
type 2 diabetes (T2D);
hypertension (HT);
ankylosing spondylitis (AS);
autoimmune thyroid disease (AITD);
multiple sclerosis (MS);
breast cancer (BC);
prostate cancer (PC);
systemic lupus erythematosus (SLE);
autoimmune diseases (AID);
and ulcerative colitis (UC).
p values were calculated using by hypergeometric distribution test.

Broadly, the analysis indicates that altered functions of the nuclear import pathway may have a significant contribution to the pathogenesis of many common human disorders. Consistent with this idea, KPNA1 expression is altered in patients diagnosed with Crohn's disease, T2D, RA, and bipolar disorder, suggesting that this knowledge can be exploited for diagnostic and therapeutic gains. Moreover, consistent with the findings of increased KPNA1 mRNA expression in kidneys of T2D patients with diabetic nephropathy and db/db mice with the experimental model of T2D diabetes (see FIG. 10), increased importin alpha protein expression in diabetic nephropathy has been reported. Despite broad recognition of importance of the nuclear import pathway, knowledge of its molecular physiology and pathology remains very limited. A recent report demonstrates that switching of importin-alpha subtypes exerts a selective gate-keeping function in the nuclear import of key transcription factors that regulate stem cell maintenance and differentiation. (See Yasuhara N., et al., Triggering neural differentiation of ES cells by subtype switching of importin-alpha. Nat. Cell Biol. 9:72-79 (2007)). Altered expression of importins in multiple human disorders may directly affect genetic and molecular mechanisms which are critically important for normal functions of the immune system. Genes for immunoglobulins and T-cell receptor are generated by a process known as V(D)J recombination. This process is highly regulated and mediated by the recombination activating proteins RAG-1 and RAG-2. RAG-1 and RAG-2 are lymphoid-specific genes that together induce V(D)J recombinase activity in a variety of nonlymphoid cell types. It has been demonstrated that importins may play a role in V(D)J recombination by directly interacting with the RAG-1 protein. (Cortes O., et al., RAG-1 interacts with the repeated amino acid motif of the human homologue of the yeast protein SRP1. Proc. Nat. Acad. Sci., 91:7633-7637 (1994) and Cuomo C. A., et al., Rch1, a protein that specifically interacts with RAG-1 recombination-activating protein. Proc. Natl. Acad. Sci. USA. 91:6156-6160 (1994)).

A disease phenocode hypothesis postulates that the effect in trans of SNP sequence-bearing RNAs on phenotypes would depend on the level of expression of SNP-harboring genetic loci. Therefore, this concept does not eliminate the important role of classic disease-associated protein-coding loci in the pathogenesis of human disorders. However, it does add a new mechanistic dimension to the understanding of how their expression may affect disease phenotypes which was previously overlooked and, perhaps, deserve further critical experimental and translational interrogation. It would be of interest to apply this approach for systematic identification and analysis, of disease-specific phenocodes and test the practical utility of this strategy for both diagnostic and therapeutic applications.

A sequence homology profiling was carried out profiling of the allelic sequences of the 93 SNP loci located at distinct chromosomal regions of human genome and manifesting most significant associations with seven common human diseases as shown in Example 1, infra.

SNP-Guided microRNA Maps (MirMaps) Of 16 Common Human Disorders Identify a Clinically Accessible Therapy Reversing Transcriptional Aberrations of Nuclear Import and Inflammasome Pathways

A disease phenocode analysis was also used to examine the relationships between structural features and gene expression patterns of disease-linked SNPs, microRNAs, and mRNAs of protein-coding genes in association to phenotypes of 16 major human disorders, enabled by multiple independent studies of up to 451,012 combined samples including 191,975 disease cases and 253,496 controls. SNP sequence homology-guided microRNA maps (“MirMaps”) identify consensus components of a disease phenocode consisting of 81 SNPs and 17 microRNAs. microRNAs of the consensus set are associated with at least 4 common human diseases (range 4 to 7 diseases) and manifest sequence homology/complementarity to at least 4 distinct disease-linked SNPs (range 4 to 14 SNPs). Nearly all microRNAs (15 of 17; 88%) of the consensus set has potential protein-coding mRNA targets among the principal components of the nuclear import pathway (NIP) and/or inflammasome pathways including KPNA1, NLRP1 (NALP1), and NLRP3 (NALP3) genes. Analysis of expression profiling experiments of peripheral blood mononuclear cells (PBMC) demonstrates statistically significant KPNA1-, NLRP1-, and NLRP3-gene expression phenotypes associated with human genotypes of Crohn's disease (CD), Huntington's disease (HD), and rheumatoid arthritis (RA) populations.

Unexpectedly, microarray analysis of PBMC from patients treated with chloroquine reveals a reversal of disease-linked KPNA1-, NLRP1-, and NLRP3-gene expression phenotypes, thereby implying that chloroquine could serve as a readily clinically available drug for targeted correction of identified aberrations. Genetically-defined malfunctions of the NIP and inflammasome pathways are likely to contribute to pathogenesis of multiple common human disorders and PBMC-based genetic tests may be useful for monitoring the individual's response to therapy. Thus, prescription of chloroquine, an FDA-approved drug which is widely utilized for treatment of malaria, RA, and systemic lupus erythematosus (SLE), may have a therapeutic value in clinical management of a large spectrum of human disorders.

As discussed, a disease phenocode hypothesis is proposed stating that DNA sequence variations associated with multiple major human disorders may affect phenotypes in trans via non-protein-coding SNP sequence-bearing RNA intermediaries. According to a disease phenocode hypothesis, one of the physiologically- and pathologically-relevant biological functions of SNP-sequence-bearing sncRNAs is the interference with activity and/or biogenesis of microRNAs, which, in turn, would affect gene expression and phenotypes. If RNA transcripts have the potential to interfere with the biogenesis and/or bioactivity of microRNAs, they must exhibit the apparent sequence homology/complementarity features to the targeted microRNAs.

Proof of principle validation of this approach identified human disease phenocodes which are reflecting sequence homology-driven associations between disease-linked SNPs, microRNAs, and mRNAs of protein-coding genes. A multi-step analytical protocol is designed to facilitate identification of primary sequence-related sets of SNPs, microRNAs, and mRNAs associated with phenotypes of interest. The validity of the disease phenocode concept was confirmed within a genomic context of distinct continuously spaced sets of disease-linked SNPs and mRNAs of relevant protein-coding genes by analyzing two sets of SNPs which are located within continuous genomic regions associated with individual protein-coding genetic loci (NLRP1 and STAT4) and are likely to exhibit common profiles of transcriptional activity. One of the important end-points derived from this approach is the identification of the principal components of the nuclear import pathway as potential common targets across the diverse spectrum of human diseases. However, one of the possible significant limitations of the previous effort is that at the discovery stage of a consensus disease phenocode a single data comprising of 17,000 combined samples including 14,000 disease cases of 7 common human disorders and 3,000 shared controls was utilized.

Sequence Homology Profiling of Disease-Linked SNPs Identifies SNP-Guided MicroRNA Maps (MirMaps) Revealing a Consensus Disease Phenocode Consisting of 81 SNPs and 17 MicroRNAs

A disease phenocode analysis was performed by developing the SNP-guided microRNA maps (“MirMaps”) of individual human disorders. For each pathological condition, disease-linked SNPs were selected which manifest most significant associations with common human disorders based on multiple independent studies of up to 451,012 combined samples including 191,975 disease cases and 253,496 controls. Included in the sequence homology profiling analysis is an original set of 93 SNPs which are most significantly associated with seven common human disorders, namely bipolar disease (BD); rheumatoid arthritis (RA); coronary artery disease (CAD); Crohn's disease (CD); type 1 diabetes (T1D); type 2 diabetes (T2D); and hypertension (HT); 23 SNPs with most significant evidence for associations with T2D (4) and 16 RA-linked SNPs. In addition, sequence homology profiling was carried out of 18 AITD-linked SNPs; 15 MS-linked SNPs; 12 SNPs associated with autoimmune disorders (AID); 20 AS-linked SNPs; 16 breast cancer (BC)-linked SNPs; 18 systemic lupus erythematosus (SLE)-linked SNPs; 18 prostate cancer (PC)-linked SNPs; 18 vitiligo-associated multiple autoimmune disease SNPs (VIT); 5 ulcerative colitis (UC)-linked SNPs; 8 colorectal cancer (CRC)-associated SNPs, all of which were identified and replicated in multiple independent studies. Analysis of individual SNP-guided microRNA maps of human diseases demonstrates in all instances the apparent propensity to target the KPNA1-encoded mRNAs by the disease-linked microRNAs, confirming that nuclear import pathway may represent a critically important target in multiple major human disorders.

At the next step of disease phenocode analysis, individual disease MirMaps were combined into a single spreadsheet representing the integral SNP-guided map of microRNAs homologous to disease-linked SNPs and selected an elite set of the top-scoring SNP/microRNA pairs with the highest numbers of sequence homology calls. (See Table 10).

TABLE 10
81 SNPs and 17 microRNAs comprising a consensus phenocode of 16 common human
disorders

| | SNP/ | | miR- | mir- | mir- | mir- | miR- | miR- | miR- |
|---|---|---|---|---|---|---|---|---|---|
| Disease | microRNA | let-7 | 548 | 125 | 520 | 519 | 181 | 1238 | 200 |
| BD | rs11622475 | | | | | | | | |
| BD | rs7570682 | 1 | | | | | | | |
| CAD | rs1333049 | | | | 1 | 1 | | | |
| CD | rs11209026 | | | 1 | | | | | |
| CD | rs11805303 | | | | | 1 | | | |
| CD | rs6908425 | | | | | | 1 | | |
| CD | rs7807268 | | | 1 | | | | | |
| CD | rs9469220 | | | | | | 1 | | |
| CD/T1D | rs2542151 | | | 1 | | | | | |
| HT | rs2398162 | | | | | | | | |
| RA | rs11761231 | | | | | | | | |
| RA | rs615672 | | | | | | | | |
| RA | rs2837960 | | | | | | | | |
| RA | rs3816587 | | | | | | | | |
| RA | rs6684865 | | | | | | 1 | | |
| RA | rs6920220 | | | | | 1 | | | |
| RA | rs9550642 | | | | | | | 1 | |
| RA/T1D | rs2476801 | | | | | | | | |
| T1D | rs3087243 | | | | | | | | |
| T1D | rs9270986 | | | | | | | | 1 |
| T1D/CD | rs2642151 | | | 1 | | | | | |
| T1D/RD | rs2476601 | | | | | | | | |
| T2D | rs13071168 | 1 | | | | | | | |
| T2D | rs1801282 | | | | 1 | 1 | | | |
| T2D | rs4402960 | 1 | | | | | | | |
| T2D | rs10282940 | 1 | | | | | | | |
| T2D | rs7020996 | | 1 | | | | | | |
| T2D | rs5016480 | | | | | | 1 | | |
| T2D | rs7903146 | | | | 1 | | | | |
| T2D | rs8050136 | | 1 | | | | | | |
| T2D | rs17705177 | | 1 | | | | | | |
| T2D | rs864745 | | 1 | | | | | | |
| T2D | rs1153188 | | | | | | | | |
| UC/AS | rs3197999 | 1 | | | | | | | |
| BC | rs2298083 | | | | | | | | |
| BC | rs4986790 | | | | | | | | |
| BC | rs7313899 | | | | | | 1 | | |
| BC | rs9616915 | | | | | | 1 | 1 | |
| BC | rs889312 | | | | | | | | |
| BC | rs1053485 | | 1 | | | | | | |
| PC | rs4430796 | | | | | | | | |
| PC | rs7501939 | | | | | | | | |
| PC | rs3760511 | 1 | | 1 | | | | | |
| PC | rs1859962 | | | 1 | | | | | |
| PC | rs983085 | | | | | | | | |
| PC | rs16901979 | | 1 | | | | | | |
| PC | rs4242382 | 1 | | | | | | | |
| PC | rs7837688 | 1 | | | | | | | |
| SLE | rs4548893 | 1 | | | | | | | |
| SLE | rs9888739 | | 1 | | | | | | |
| SLE | rs10516487 | 1 | | | | | | | |
| SLE | rs17266594 | | | | | | | | |
| SLE | rs11574637 | | | 1 | | | | | |
| VIT | rs12150220 | 1 | | | | | | | |
| VIT | rs7223628 | | | | | | | | |
| VIT | rs8182354 | | | | 1 | 1 | | | |
| VIT | rs925597 | | | | | | | | |
| VIT | rs961826 | 1 | 1 | | | | | | |
| MS | rs6957669 | | | 1 | | | | | |
| MS | ATM-333 | | | | 1 | | | | |
| MS | rs7162473 | | | | | | | | |
| MS | rs2229358 | | | | | | | | 1 |
| MS | rs11554159 | | | | | | | | |
| MS | rs1800437 | | | | | | | | |
| AITD | rs35285785 | | | | | | | 1 | |
| AITD | rs7578199 | | | | | | | | |
| AITD | rs7302981 | | | | | | | | |
| AITD | rs7975069 | | | | | | | | 1 |
| AS | rs12143301 | 1 | | | | | | | |
| AS/UC | rs3197999 | 1 | | | | | | | |
| AS | rs27044 | | | | | | | 1 | |
| AS | rs30187 | | | | 1 | | | | |
| AS | rs2302250 | | | | | | | | |
| AS | rs9939768 | | | 1 | | | | | |
| AS | rs11554159 | | | | | | | | |
| AS | rs709012 | | | | | | | | |
| AID | rs12067507 | | 1 | | | | | 1 | |
| AID | rs1729674 | | 1 | | | | | | |
| AID | rs2232337 | | 1 | | | | | | |
| AID | rs11171 | | | | | | | | |
| AID | rs11674422 | | | | | | | | |
| AID | rs34536443 | | | | | | | | |
| AID | rs2303759 | | | | | | 1 | | 1 |
| CRC | rs4779584 | | 1 | | | | | | |
| | SCORE, | 14 | 12 | 9 | 6 | 5 | 7 | 5 | 4 |
| | SNP | | | | | | | | |
| | SCORE, | 7 | 7 | 6 | 5 | 5 | 5 | 5 | 5 |
| | DISEASES | | | | | | | | |
| | | T2D | T2D | CD | CAD | CAD | CD | RA | T1D |
| | | PC | PC | T1D | T2D | CD | RA | BC | AITD |
| | | SLE | VIT | PC | MS | RA | T2D | AITD | AID |
| | | VIT | SLE | MS | AS | T2D | BC | AS | MS |
| | | AS | BC | AS | VIT | VIT | AID | AID | MS |
| | | BD | AID | SLE | | | | | |
| | | UC | CRC | | | | | | |
| | microRNA | let-7 | miR- | mir- | mir- | mir- | miR- | miR- | miR- |
| | homolog | | 548 | 125 | 520 | 519 | 181 | 1238 | 200 |
| | P value | 1.5E−08 | 1.5E−08 | 6.3E−07 | 1.9E−05 | 1.9E−05 | 1.9E−05 | 1.9E−05 | 1.9E−05 |

| | miR- | miR- | mir- | miR- | mir- | miR- | miR- | miR- | miR- |
|---|---|---|---|---|---|---|---|---|---|
| Disease | 143 | 374 | 146 | 509 | 602 | 23 | 541 | 612 | 662 |
| BD | | | | | | 1 | | | |
| BD | | | | | | | | | |
| CAD | | | | | | | | | |
| CD | | | | | | | | | |
| CD | | | | | | | | | |
| CD | | | | | | | | | |
| CD | | | | | | | | | |
| CD | | 1 | | | | | | | |
| CD/T1D | | | | | | | | | |
| HT | | | | | | | | 1 | |
| RA | | | | | | | | 1 | |
| RA | | | | | | | 1 | | |
| RA | | | | 1 | 1 | | | | |
| RA | | | 1 | | | | | | |
| RA | | | | | | | | | |
| RA | | | | | | | | | |
| RA | | | | | | | | | |
| RA/T1D | | | | | | | 1 | | |
| T1D | | | | 1 | | | | | 1 |
| T1D | | | | | | | | | |
| T1D/CD | | | | | | | | | |
| T1D/RD | | | | | | | 1 | | |
| T2D | | | | | | | | | |
| T2D | | | | | | | | | |
| T2D | | | | | | | | | |
| T2D | | | | | | | | | |
| T2D | | | | | | | | | |
| T2D | | | | | | | | | |
| T2D | | | | | | | | | |
| T2D | | | | 1 | | | | | |
| T2D | | | | | | | | | |
| T2D | | | | | | | | | |
| T2D | | | 1 | | | | | | |
| UC/AS | | | | | 1 | | | | |
| BC | | | | | | | | 1 | |
| BC | | 1 | | | | | | | |
| BC | | 1 | | | | | | | |
| BC | | | | | | | | | |
| BC | | | 1 | | | | | | |
| BC | | | | | | | | | |
| PC | 1 | | | | | | | | |
| PC | 1 | | | | | | | | |
| PC | | | | | | | | | |
| PC | | | | | | 1 | | | |
| PC | | 1 | | | | | | | |
| PC | | | | | | | | | |
| PC | | | | | | | | | |
| PC | | | | | | | | | |
| SLE | | | | | | | | | |
| SLE | | | | | | | | | |
| SLE | | | | | | | | | |
| SLE | | 1 | | | | | | | |
| SLE | | | | | | | | | |
| VIT | | | | | | | | | |
| VIT | | | | | | | 1 | | |
| VIT | | | | | | | | | |
| VIT | | | | | 1 | | | | |
| VIT | | | | | | | | | |
| MS | | | | | | | | | |
| MS | | | | | | | | | |
| MS | | | | | | 1 | | | |
| MS | | | | | | | | | |
| MS | 1 | | | | | | | | 1 |
| MS | | | | | | 1 | | | |
| AITD | | | | | | | | | |
| AITD | 1 | | | | | | | | |
| AITD | | | 1 | | | | | | |
| AITD | | | | | | | | | |
| AS | | | | | | | | | |
| AS/UC | | | | | 1 | | | | |
| AS | | | | | | | | | |
| AS | | | | | | | | | |
| AS | 1 | | | | | | | | |
| AS | | | | | | | | | |
| AS | 1 | | | | | | | | 1 |
| AS | | | | 1 | | | | | |
| AID | | | | | | | | | |
| AID | | | | | | | | | |
| AID | | | | | | | | | |
| AID | | | | | | | 1 | | |
| AID | | | | | | 1 | | | |
| AID | | | | | | | | 1 | 1 |
| AID | | | | | | | | | |
| CRC | | | | | | | | | |
| | 6 | 5 | 4 | 4 | 4 | 5 | 5 | 4 | 4 |
| | 4 | 4 | 4 | 4 | 4 | 4 | 4 | 4 | 4 |
| | MS | CD | RA | RA | UC | BD | RA | HT | MS |
| | AITD | BC | T2D | T1D | VIT | PC | T1D | RA | AS |
| | AS | PC | BC | T2D | AS | MS | AID | BC | AID |
| | PC | SLE | AITD | AS | RA | AID | VIT | AID | T1D |
| | miR- | miR- | mir- | miR- | mir- | miR-23 | miR- | miR- | miR- |
| | 143 | 374 | 148 | 509 | 602 | | 541 | 612 | 682 |
| | 0.00039 | 0.00039 | 0.00039 | 0.00039 | 0.00039 | 0.00039 | 0.00039 | 0.00039 | 0.00039 |

Score numbers represent the sum of sequence homology profiling-defined association events of a given microRNA and disease-linked SNPs.
Human disorders:
BD, bipolar disease;
RA, rheumatoid arthritis;
CAD, coronary artery disease;
CD, Crohn's disease;
T1D, type 1 diabetes;
T2D, type 2 diabetes;
HT, hypertension;
AS, ankylosing spondylitis;
AITD, autoimmune thyroid disease;
MS, multiple sclerosis;
BC, breast cancer;
PC, prostate cancer;
CRC, colorectal cancer;
SLE, systemic lupus erythematosus;
AID, autoimmune diseases;
UC, ulcerative colitis.
p values were calculated using hypergeometric distribution test. Scored sequence homology events between SNPs and microRNAs are designated by the number 1 in the table.

As shown in Table 10, a systematic primary sequence homology-driven pattern of associations between disease-linked SNPs and microRNAs reveals a consensus SNP-guided MirMap of human diseases consisting of 81 SNPs and 17 microRNAs. microRNAs of the consensus set are associated with at least 4 common human diseases (range 4 to 7 diseases; see Table 10) and manifest sequence homology and/or complementarity to at least 4 distinct disease-linked SNPs (range 4 to 14 SNPs; see Table 10). Moreover, the probability that multiple sequence homology calls occurred by chance was estimated and found that it is highly unlikely (Table 10).

Next, whether a consensus set of 17 microRNAs would have the propensity to target mRNAs of importin genes, which were recently identified as potential targets in several human diseases was determined. The lists of importin-targeting microRNAs were retrieved and predicted mRNA targets for each of the 17 microRNA listed in Table 10 using TargetScan database and searched for concordant sets of microRNAs and mRNA targets. The analysis reveals that 88% of identified microRNAs (see Table 11) have the potential to target mRNA sequences derived from importin genes.

TABLE 11
Importin mRNA-targeting map of the 17 microRNAs comprising a consensus phenocode
of 16 human disorders

| microRNA | KPNA1 | KPNA2 | KPNA3 | KPNA4 | KPNA5 | KPNA6 | KPNB1 | KPNB2 |
|---|---|---|---|---|---|---|---|---|
| let-7 | let-7 | | | let-7 | | let-7 | | |
| mir-125 | | | | | | mir-125 | mir-125 | |
| miR-548 | miR-548 | | miR-548 | miR-548 | miR-548 | miR-548 | miR-548 | miR-548 |
| mir-520 | mir-520 | mir-520 | mir-520 | | | mir-520 | mir-520 | mir-520 |
| mir-519 | mir-519 | | mir-519 | | | | | |
| miR-181 | miR-181 | | | miR-181 | | | miR-181 | miR-181 |
| miR-1238 | | | | | | | | |
| miR-200 | | | miR-200 | miR-200 | | miR-200 | | miR-200 |
| miR-143 | miR-143 | | | | | miR-143 | | |
| miR-374 | miR-374 | | | | | miR-374 | | miR-374 |
| mir-146 | mir-146 | | | | | mir-146 | | |
| miR-509 | | | | | | | | |
| mir-602 | | | | | | | | |
| miR-23 | miR-23 | | miR-23 | miR-23 | | | | miR-23 |
| miR-541 | miR-541 | | | | | | | |
| miR-612 | miR-612 | | | | | miR-612 | | |
| miR-662 | | | | | | miR-662 | | |
| Score, | 11 | 1 | 5 | 5 | 1 | 10 | 4 | 6 |
| microRNA | | | | | | | | |
| P values | 0.0006609 | 0.360965 | 0.049809429 | 0.042488 | 0.33156 | 0.00158991 | 0.045375 | 0.086413 |

| microRNA | KNPB3 | SCORE | DISEASES | | | | | | |
|---|---|---|---|---|---|---|---|---|---|
| let-7 | let-7 | 7 | T2D | PC | SLE | VIT | AS | BD | UC |
| mir-125 | | 6 | CD | T1D | PC | MS | AS | SLE | |
| miR-548 | miR-548 | 7 | T2D | PC | VIT | SLE | BC | AID | CRC |
| mir-520 | mir-520 | 5 | CAD | T2D | MS | AS | VIT | | |
| mir-519 | | 5 | CAD | CD | RA | T2D | VIT | | |
| miR-181 | miR-181 | 5 | CD | RA | T2D | BC | AID | | |
| miR-1238 | | 5 | RA | BC | AITD | AS | AID | | |
| miR-200 | miR-200 | 5 | T1D | AITD | AID | MS | MS | | |
| miR-143 | | 4 | MS | AITD | AS | PC | | | |
| miR-374 | miR-374 | 4 | CD | BC | PC | SLE | | | |
| mir-146 | | 4 | RA | T2D | BC | AITD | | | |
| miR-509 | miR-509 | 4 | RA | T1D | T2D | AS | | | |
| mir-602 | | 4 | UC | VIT | AS | RA | | | |
| miR-23 | miR-23 | 4 | BD | PC | MS | AID | | | |
| miR-541 | | 4 | RA | T1D | AID | VIT | | | |
| miR-612 | | 4 | HT | RA | BC | AID | | | |
| miR-662 | | 4 | MS | AS | AID | T1D | | | |
| Score, | 8 | | | | | | | | |
| microRNA | | | | | | | | | |
| P values | 0.002347 | | | | | | | | |

microRNA scores represent the number of microRNAs with potential to target mRNAs encoded by a given importin gene. Disease score numbers represent the sum of sequence homology profiling-defined association events of a given microRNA and disease-linked SNPs.
Human disorders:
BD, bipolar disease;
RA, rheumatoid arthritis;
CAD, coronary artery disease;
CD, Crohn's disease;
T1D, type 1 diabetes;
T2D, type 2 diabetes;
HT, hypertension;
AS, ankylosing spondylitis;
AITD, autoimmune thyroid disease;
MS, multiple sclerosis;
BC, breast cancer;
PC, prostate cancer;
CRC, colorectal cancer;
SLE, systemic lupus erythematosus;
AID, autoimmune diseases;
UC, ulcerative colitis.
Human importin-targeting microRNAs were identified using TargetScan database. p values were calculated using hypergeometric distribution tests. They represent the estimates of the likelihood of obtaining score values by chance and take into account the numbers of all screened for homology microRNAs and the number of microRNAs which are predicted to target a given importin gene.

Moreover, all 81 disease-linked SNPs listed in Table 10 manifest sequence homology to microRNAs which are predicted to target mRNAs of importin genes (see Table 11), indicating that sequence homology to the importin-targeting microRNAs is a common structural feature of many SNPs associated with multiple major human diseases.

mRNAs of the Principal Components of Inflammasome Pathways are Potential Targets of the Consensus Disease Phenocode microRNAs

Recent experimental observations implicate components of inflammasome pathways and innate immune system in the pathogenesis of multiple autoimmune and autoinflammatory disorders. (See Jin Y. et al., NALP1 in vitiligo-associated multiple autoimmune disease. N. Engl. J. Med. 356:1216-1225 (2007)). However, the underlying molecular causes of the inflammasome malfunction in human diseases remain obscure. Thus, a disease phenocode analysis was applied to look for changes of the microRNA targeting potency against mRNAs of the inflammasome-related genes. The lists of microRNAs were obtained with targeting potentials against nine inflammasome-related genes (see Table 12) and the predicted mRNA targets for each of the 17 microRNA listed in Table 10 were obtained using TargetScan database and searched for using concordant sets of microRNAs and mRNA targets.

TABLE 12
Inflammasome mRNA-targeting map of the 17 microRNAs comprising a consensus
phenocode of 16 human disorders

| mRNA/ | | | | | | | | |
|---|---|---|---|---|---|---|---|---|
| microRNA | NLRP1 | NLRP2 | NLRP3 | NLRP4 | NLRP5 | NLRP7 | NLRP8 | NLRP9 |
| let-7 | | | | | | | | |
| mir-125 | mir-125 | mir-125 | | | | | | |
| miR-548 | miR-548 | | miR-548 | miR-548 | | | | |
| mir-520 | mir-520 | | mir-520 | | | | mir-520 | |
| mir-519 | | | | | | | | |
| miR-181 | | | | | | | miR-181 | |
| miR-1238 | | | | | | | | |
| miR-200 | | | miR-200 | miR-200 | | | | |
| miR-143 | miR-143 | | | | | | | |
| miR-374 | | | | | | | | |
| mir-146 | | | | | | | | |
| miR-509 | | | | | | | | |
| mir-602 | | | | | | | | |
| miR-23 | | | | | | | | |
| miR-541 | | | | | | | | |
| miR-612 | | | | | | | | |
| miR-662 | miR-662 | | | | | | | |
| Score, | 5 | 1 | 3 | 2 | 0 | 0 | 2 | 0 |
| microRNA | | | | | | | | |
| P values | 0.000672 | 0.267683 | 0.036323 | 0.032745 | 0.516475 | 0.763468 | 0.140624 | 0.636009 |

| mRNA/ | | | | | | | | | |
|---|---|---|---|---|---|---|---|---|---|
| microRNA | NLRP10 | SCORE | DISEASES | | | | | | |
| let-7 | | 7 | T2D | PC | SLE | VIT | AS | BD | UC |
| mir-125 | | 6 | CD | T1D | PC | MS | AS | SLE | |
| miR-548 | | 7 | T2D | PC | VIT | SLE | BC | AID | CRC |
| mir-520 | | 5 | CAD | T2D | MS | AS | VIT | | |
| mir-519 | | 5 | CAD | CD | RA | T2D | VIT | | |
| miR-181 | | 5 | CD | RA | T2D | BC | AID | | |
| miR-1238 | | 5 | RA | BC | AITD | AS | AID | | |
| miR-200 | | 5 | T1D | AITD | AID | MS | MS | | |
| miR-143 | | 4 | MS | AITD | AS | PC | | | |
| miR-374 | | 4 | CD | BC | PC | SLE | | | |
| mir-146 | | 4 | RA | T2D | BC | AITD | | | |
| miR-509 | | 4 | RA | T1D | T2D | AS | | | |
| mir-602 | | 4 | UC | VIT | AS | RA | | | |
| miR-23 | | 4 | BD | PC | MS | AID | | | |
| miR-541 | | 4 | RA | T1D | AID | VIT | | | |
| miR-612 | | 4 | HT | RA | BC | AID | | | |
| miR-662 | | 4 | MS | AS | AID | T1D | | | |
| Score, | 0 | | | | | | | | |
| microRNA | | | | | | | | | |
| P values | 0.914459 | | | | | | | | |

microRNA scores represent the number of microRNAs with potential to target mRNAs encoded by a given NLRP gene. Disease score numbers represent the sum of sequence homology profiling-defined association events of a given microRNA and disease-linked SNPs.
Human disorders:
BD, bipolar disease;
RA, rheumatoid arthritis;
CAD, coronary artery disease;
CD, Crohn's disease;
T1D, type 1 diabetes;
T2D, type 2 diabetes;
HT, hypertension;
AS, ankylosing spondylitis;
AITD, autoimmune thyroid disease;
MS, multiple sclerosis;
BC, breast cancer;
PC, prostate cancer;
CRC, colorectal cancer;
SLE, systemic lupus erythematosus;
AID, autoimmune diseases;
UC, ulcerative colitis.
Human inflammasome-targeting microRNAs were identified using TargetScan database. p values were calculated using hypergeometric distribution tests. They represent the estimates of the likelihood of obtaining score values by chance and take into account the numbers of all screened for homology microRNAs and the number of microRNAs which are predicted to target a given importin gene.

Forty one percent of consensus microRNAs shown in Table 12 have the potential to target mRNA sequences derived from inflammasome-related genes. The probability of the occurrence of multiple sequence homology calls by chance and found that predicted targeting effect on mRNAs of six NLRP genes did not reach thresholds of statistical significance to exclude the likelihood of occurrence of multiple calls by chance (see Table 12). A consensus set of 17 microRNAs appears to have the propensity to target mRNAs of the selected inflammasome-related genes, namely NLRP1, NLRP3, NLRP4. Of note, both NLRP1 and NLRP3 genes are the principal components of the corresponding NLRP1- and NLRP3-inflammasomes and NLRP4 protein modulates NF-kappa B induction by inflammatory cytokines, in particular, by the interleukin-1-beta, production of which is increased during inflammasome activation. (See Fiorentino L. et al., A novel PAAD-containing protein that modulates NF-kappaB induction by cytokines tumor necrosis factor-alpha and interleukin-1-beta. J. Biol. Chem. 277:35333-40 (2002)).

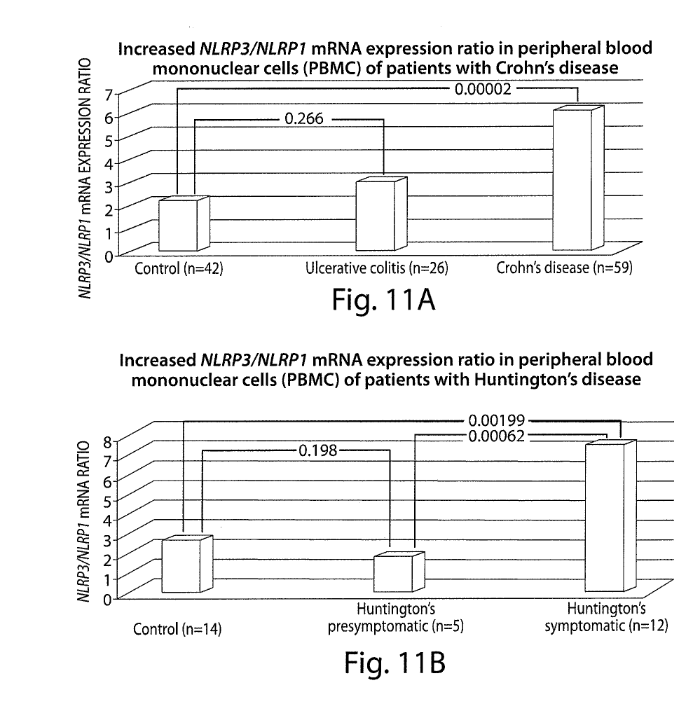

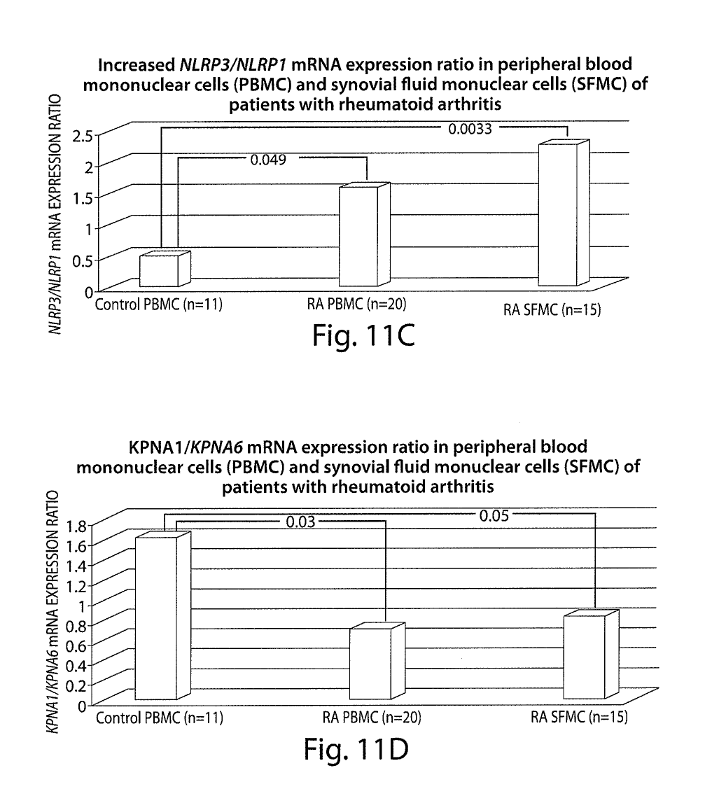

Microarray analysis reveals common gene expression changes in the peripheral blood mononuclear cells (PBMC) of CD and RA patients constituting a decreased NLRP1 mRNA expression and an increased NLRP3 mRNA expression. Gene expression profiling experiments indicate that altered expression phenotypes of the principal inflammasome components common for CD and RA patients is also evident in patients with symptomatic Huntington's disease (HD). Microarray analysis demonstrates statistically significant increased expression of the NLRP3 mRNAs and decreased expression of the NLRP1 mRNAs in PBMC of patients with Huntington's disease (See, FIGS. 11A-D). Consequently, NLRP3/NLRP1 mRNA expression ratio in PBMC of CD, RA, HD patients is increased by 2.8-fold, 4.5-fold, and 2.8-fold, respectively.

Chloroquine Therapy Reverses Disease-Associated Gene Expression Phenotypes of Nuclear Import and Inflammasome Pathways

As noted, a common pattern of altered gene expression of the principal components of inflammasome pathways in PBMC of CD, RA, and HD patients was observed, thereby suggesting that these findings can be exploited for development of a simple blood-based surrogate marker test for diagnostic and therapy selection and monitoring applications. Moreover, these findings could also be utilized to search for potential therapeutics by looking for drugs, which would cause a reversal of identified disease-associated gene expression phenotypes.

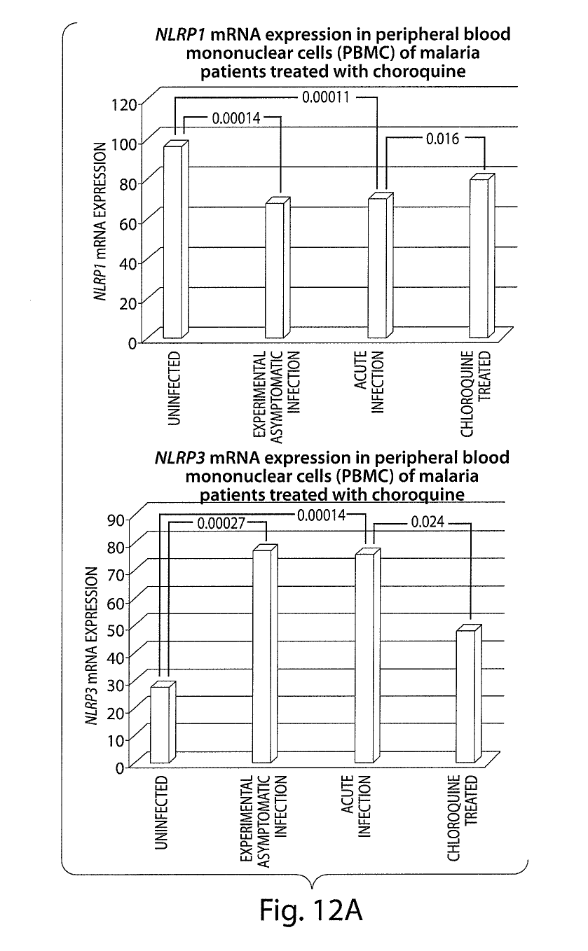

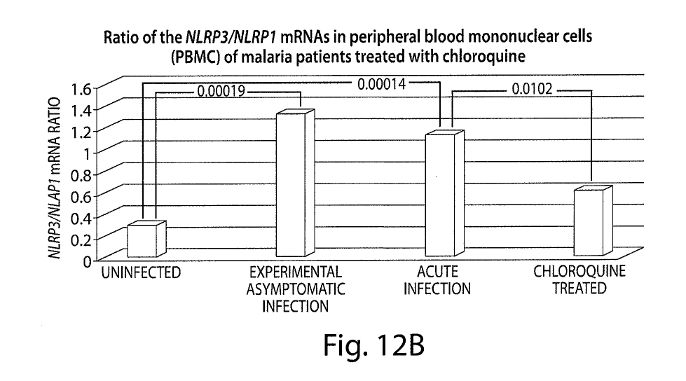

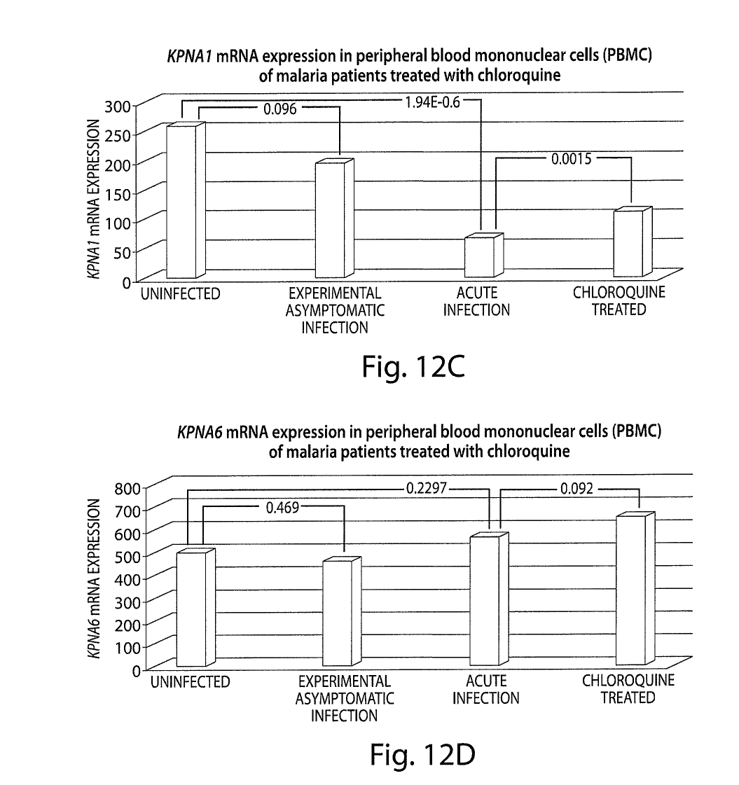

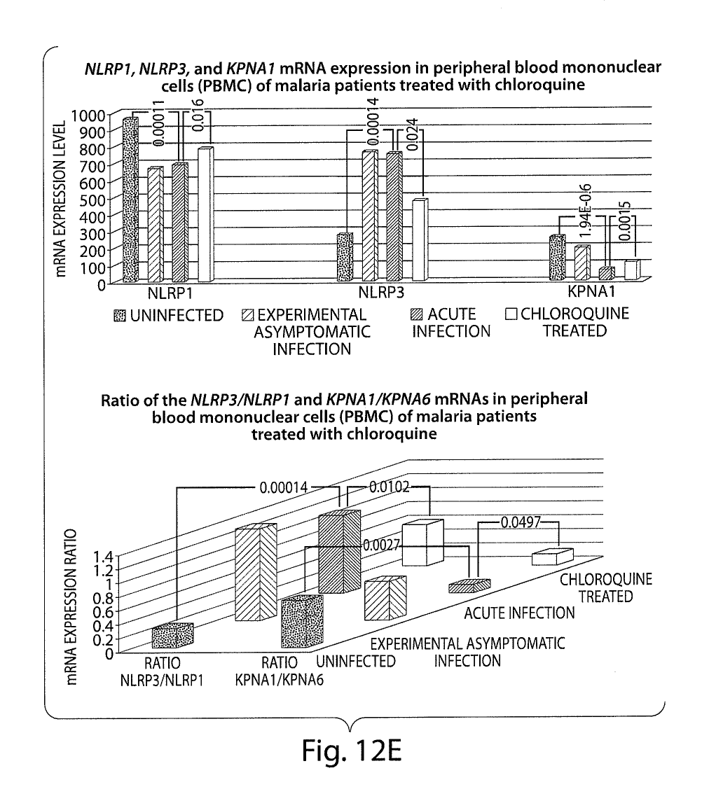

Remarkably, microarray analysis of PBMC from malaria patients treated with chloroquine revealed that chloroquine therapy appears to reverse disease-associated mRNA expression changes of the KPNA1, NLRP1, and NLRP3 genes. (See FIG. 12). As shown in FIG. 12, decreased expression levels of the KPNA1 (3.9-fold) and NLRP1 (1.4-fold) mRNAs were elevated by 66% (p=0.0015) and 13% (p=0.016), respectively; whereas increased expression of the NLRP3 mRNA (2.7-fold) was reduced by 57% (p=0.024) after chloroquine therapy. In contrast, expression levels of the KPNA6 mRNA as well as multiple other importins and inflammasome-related genes do not manifest statistically significant changes. (See FIG. 12).

Consequently, the 3.8-fold (p=0.00014) elevated NLRP3/NLRP1 mRNA expression ratio is reduced by 1.9-fold (p=0.0102) after chloroquine therapy. (See FIG. 12). Conversely, the 5.6-fold (p=0.0027) decreased KPNA1/KPNA6 mRNA expression ratio is increased by 1.4-fold (p=0.0497) after administration of chloroquine therapy. (See FIG. 12). PBMC from malaria patients treated with chloroquine manifest drug-induced partial reversal of the aberrant gene expression phenotypes of the principal components of nuclear import and inflammasome pathways.

Disease Phenocode Hypothesis

Elucidation of genetic causes of human diseases should enable the precise molecular understanding of how genetic variations contribute to pathological phenotypes. A dominant concept remains to consider the potential effects of sequence variations on protein-coding host genes or nearby genetic loci. Recently, a novel strategy has emerged which takes into account the SNP variants residing within boundaries of genes encoding microRNAs and SNPs within microRNA-target sites in mRNAs. Many statistically most significant disease-associated SNPs are located within introns, integenic, and non-genic regions of a genome, suggesting that alternative non-orthodox mechanisms linking SNP variations to disease phenotypes should be considered.

Thus, it was hypothesized that DNA sequence variations associated with multiple major human disorders may affect phenotypes in trans via non-protein-coding RNA intermediaries which would interfere with functions and/or biogenesis of microRNAs and affect gene expression. If RNA transcripts have the potential to interfere with the biogenesis and/or bioactivity of microRNAs, they must exhibit the apparent sequence homology/complementarity features to the targeted microRNAs. Proof of principle validation of this approach identified phenocodes of several human diseases reflecting sequence homology-driven associations between disease-linked SNPs, microRNAs, and mRNAs of protein-coding genes. A disease phenocode concept employs the multi-step analytical protocol facilitating identification of a set of SNPs, microRNAs, and mRNAs associated with phenotypes of interest. One of the significant end-points derived from this approach is the identification of the principal components of the nuclear import pathway as potential common targets across the diverse spectrum of human diseases. However, one of limitations of the previous effort is that at the discovery stage of a consensus disease phenocode a single data comprising of 17,000 combined samples including 14,000 disease cases of 7 common human disorders and 3,000 shared controls was utilized. It is formally possible that results of analysis of even such a large data set derived from a single study may have unanticipated analytical and/or methodological biases.

The recent dramatic expansion of the volume of samples and a spectrum of diseases analyzed in GWA studies was utilized to carry out the robust and stringent evaluation of the validity and utility of a disease phenocode concept in a broad clinically relevant context of human pathological conditions. Here, a disease phenocode analysis of pathology-linked SNPs was reported which manifest significant associations with 16 common human disorders based on multiple independent studies of up to 451,012 combined samples including 191,975 disease cases and 253,496 controls. The current analysis refines a systematic primary sequence homology-driven pattern of associations between disease-linked SNPs, microRNAs, and protein-coding mRNAs which is defined here as a consensus disease phenocode consisting of 81 SNPs and 17 microRNAs. It was determined that microRNAs of the consensus set are associated with at least 4 common human diseases (range 4 to 7 diseases) and manifest sequence homology/complementarity to at least 4 distinct disease-linked SNPs (range 4 to 14 SNPs).

A majority of the consensus disease phenocode microRNAs have the potentials to target mRNAs of genes constituting the principal components of the nuclear import and inflammasome pathways. microRNAs with targeting potentials against mRNAs of the KPNA1, KPNA6, NLRP1, and NLRP3 genes appear to form a statistically overlapping network.

One of the end-points of analytical definition of disease phenocodes based on a systematic primary sequence homology-driven pattern of associations between disease-linked SNPs, microRNAs, and mRNAs of protein-coding genes is the identification of a consensus SNP-guided MirMap of human diseases (Table 10). Comparisons of the previously reported MirMaps and those identified in this study reveal a significant level of consistency despite differences in analytical approaches and sample sizes utilized to generate the input lists of disease-linked SNPs (220,124 and 451,012 samples in the previous and current studies, respectively). There are 12 microRNAs (71% overlap; p=5.93E-18) and 58 SNPs (72% overlap; p=1.15E-24) in common between two consensus human disease phenocode MirMaps shown in Table 8 (Table 8: 72 SNPs and 18 microRNAs comprising a consensus phenocode of 15 common human disorders) and Table 10 (Table 10: 81 SNPs and 17 microRNAs comprising a consensus phenocode of 16 common human disorders).

A consensus set of 17 microRNAs appears to have the propensity to target mRNAs of importin genes which were recently identified as potential targets in several human diseases. 88% of identified microRNAs (see Table 11) have the potential to target mRNA sequences derived from importin genes. Moreover, all 81 disease-linked SNPs listed in Table 10 manifest sequence homology to microRNAs which are predicted to target mRNAs of importin genes (see Table 11), indicating that sequence homology to the importin-targeting microRNAs is a common structural feature of many SNPs associated with multiple major human diseases.

Therapeutic Implications of the Inflammasome Pathway Activation in Multiple Human Disorders

NLRP1 (NALP1) gene is responsible for activation of the innate immune system in response to bacterial peptides. NLRP1 is a key component of a multi-protein complex named the NLRP1 inflammasome, which also contain the adapter protein ASC and caspases 1 and 5. NALP1 also appears to play a role in activation of caspase-mediated apoptosis in a variety of cell types. NLRP3 (NALP3/CIAS1) gene product is a key component of a multi-protein complex termed the NLRP3 inflammasome. In response to pathogen challenge inflammasomes activate the proinflammatory cytokine interleukin-1(3 and trigger inflammation. Mutations in several inflammasome-related genes (NLRP); NLRP3; NOD2) are associated with multiple autoimmune/autoinflammatory diseases, suggesting interleukin-1β pathway activation and malfunction of the innate immune system. Consistent with this hypothesis, the administration of an interleukin-10 inhibitor or a caspase-1 inhibitor appears clinically beneficial in patients with these disorders. (See Hawkins P. N., et al., Interleukin-1β-receptor antagonist in the Muck-Wells syndrome. N. Engl. J. Med. 348:2583-2584 (2006); Goldbach-Mansky R., et al., Neonatal-onset multisystem inflammatory disease responsive to interleukin-1β inhibition. N. Engl. J. Med. 355:581-292 (2006); and Stach J. H., IL-converting enzyme/capase-1 inhibitor VX-765 blocks the hypersensitive response to an inflammatory stimulus in monocytes from familial cold autoinflammatory syndrome in patients. J. Immunol. 175:2630-2634 (2005)). The data suggests that interleukin-1β and caspase inhibitors might be effective in the treatment of multiple human disorders with autoimmune and autoinflammatory components of disease pathogenesis.

Nearly all microRNAs (15 of 17; 88%) of the consensus set have potential protein-coding mRNA targets among the principal components of the nuclear import pathway (NIP) and/or inflammasome/innate immunity pathways including KPNA1, NLRP1, and NLRP3 gene, thereby suggesting that malfunctions of these pathways may constitute an important element of pathogenesis of multiple human disorders.

Unexpectedly, gene expression profiling of PBMC from malaria patients treated with chloroquine reveals a reversal of disease-linked KPNA1-, NLRP1-, and NLRP3-gene expression phenotypes. These data suggest that chloroquine could serve as a clinically available drug for targeted correction of identified aberrations. It will be of interest to determine whether prescription of chloroquine, an FDA-approved drug which is broadly utilized for treatment of malaria, RA, and SLE, is therapeutically useful in clinical management of the larger spectrum of human disorders.

Increasing evidence in support of phenotype-defining functions of small non-coding RNAs (sncRNAs) prompted a conceptual recognition of informasomes as regulatory RNP complexes of sncRNAs with Argonaute proteins which are mediating information processing, alignment, and integration functions during the flow of genetic information in a cell. In support of the idea that informasomes represent stable structurally-defined organelles, recent experiments demonstrate that most of the endogenous microRNAs are tightly bound to RISC complexes in vivo and only a very small proportion of them are free in cells. (See Tang F., et al., microRNAs are tightly associated with RNA-induced gene silencing complexes in vivo. Biochem. Biophys. Res. Commun. 372:24-29 (2008). Informasome malfunctions may contribute to pathogenesis of multiple common human disorders with autoimmune/autoinflammatory components, which suggests that therapeutic strategies aimed at targeted informasome reprogramming from pathology-enabling states to physiological conditions. A fully competent microRNA biogenesis pathway is necessary to preserve regulatory T cell functions under inflammatory conditions.

A sequence homology profiling was carried out of the allelic sequences of the 93 SNP loci located at distinct chromosomal regions of human genome and manifesting most significant associations with seven common human diseases as shown in Example 1, infra.

Disease Phenocode Analysis Identifies SNP-Guided MicroRNA Maps (MirMaps) and Gene Expression Signatures Associated with Human “Master” Disease Genes

The results of a genome-wide disease phenocode analysis examining the relationships between structural features and gene expression patterns of disease-linked SNPs, microRNAs, and mRNAs of protein-coding genes in association to phenotypes of 15 common human disorders was recently reported. (See Glinsky G., Disease phenocode analysis identifies SNP-guided microRNA maps (MirMaps) associated with human “master” disease genes, Cell Cycle, 7:2570-83 (2008)). One of the main implications of this analysis is that transcriptionally co-regulated SNP sequence-bearing RNAs are more likely to exert a cumulative effect in trans on phenotypes.

The validity of a disease phenocode concept was tested within a genomic context of distinct continuously spaced sets of disease-linked SNPs and mRNAs of relevant protein-coding genes. A sequence homology profiling of two sets of disease-linked SNPs which are located within continuous genomic regions associated with individual protein-coding genetic loci (NLRP1 and STAT4) was reported and are likely to exhibit common profiles of transcriptional activity. Most of microRNAs (15 of 19; 79%) homologous to the NLPRP1-associated disease-linked SNPs have potential protein-coding mRNA targets among the principal components of the nuclear import pathway (NIP) and/or inflammasome pathways, including KPNA1, NLRP1, and NLRP3 genes. Estimates of cumulative targeting effects of microRNAs on mRNAs within distinct allelic contexts of disease-linked SNPs are in agreement with microarray analysis-defined gene expression phenotypes associated with human genotypes of Crohn's disease (CD) and rheumatoid arthritis (RA) populations. Microarray experiments and disease phenocode analysis identify ten-gene expression signatures which seem to reflect the activated status of disease-linked SNPs/microRNAs/mRNAs axis in peripheral blood mononuclear cells (PBMC) of 66% CD patients and 80% RA patients.

Comparisons of ten-gene signature expression profiles and NLRP3/NLRP1 mRNA expression ratios in PBMC of individual CD and RA patients and control subjects indicate that measurements of these markers may be useful for diagnostic applications. NLPRP1- and STAT4-associated disease-linked SNPs have common sequence-defined features, which recapitulate the essential phenotype-affecting features of genome-wide disease-linked SNPs, thereby suggesting that NLRP1 (NALP1) and STAT4 genetic loci may constitute “master” disease genes. Thus, it was concluded that both genome-wide SNP variations and SNP polymorphisms associated with “master” disease genes may cause similar genetically-defined malfunctions of the NIP and inflammasome/innate immunity pathways which are likely to contribute to pathogenesis of multiple common human disorders.

DNA sequence variations associated with multiple major human disorders may affect phenotypes in trans via non-protein-coding RNA intermediaries, which would interfere with functions and/or biogenesis of microRNAs and affect gene expression. It was reasoned that if RNA transcripts have the potential to interfere with the biogenesis and/or bioactivity of microRNAs, they must exhibit the apparent sequence homology/complementarity features to the targeted microRNAs. Proof of principle validation of this approach identified phenocodes of several human diseases reflecting sequence homology-driven associations between disease-linked SNPs, microRNAs, and mRNAs of protein-coding genes. A disease phenocode concept employs a multi-step analytical protocol facilitating identification of a set of SNPs, microRNAs, and mRNAs associated with phenotypes of interest. One of the significant end-points derived from this approach is identification of the principal components of the nuclear import pathway as potential common targets across a diverse spectrum of human diseases.

Recently, the volume of samples and a spectrum of diseases analyzed in GWAS have been expanded dramatically. These advances were used to carry out the robust and stringent evaluation of the validity and utility of a disease phenocode concept in a broad clinically relevant context of human pathological conditions. It was reported that a disease phenocode analysis of pathology-linked SNPs which manifest significant associations with 16 common human disorders based on multiple independent studies of up to 451,012 combined samples including 194,258 disease cases and 256,754 controls. (See Glinsky G. V., SNP-guided microRNA maps (MirMaps) of 16 common human disorders identify a clinically-accessible therapy reversing transcriptional aberrations of nuclear import and inflammasome pathways. Cell Cycle. 7:2570-2583 (2008)). The analysis refined a systematic primary sequence homology-driven pattern of associations between disease-linked SNPs, microRNAs, and protein-coding mRNAs which was defined as a consensus disease phenocode consisting of 81 SNPs and 17 microRNAs. Moreover, it was found that microRNAs of the consensus set are associated with at least 4 common human diseases (range 4 to 7 diseases) and manifest sequence homology/complementarity to at least 4 distinct disease-linked SNPs (range 4 to 14 SNPs). Nearly all microRNAs (15 of 17; 88%) of the consensus set have potential protein-coding mRNA targets among the principal components of the nuclear import pathway (NIP) and/or inflammasome pathways, including KPNA1, NLRP1, and NLRP3 genes.

One of the key elements of the disease phenocode hypothesis is a prediction that phenotype-altering effects in trans of SNP sequence-bearing RNAs would depend on level of expression of SNP-harboring genetic loci and transcriptionally co-regulated SNP sequence-bearing RNAs are more likely to exert a cumulative effect on phenotypes. Tiling array genome-wide expression profiling studies indicate that expression of non protein-coding RNAs are coincidental with corresponding protein-coding genetic loci, suggesting a common mechanism of transcriptional regulation. In this work, the validity of a disease phenocode concept was confined within a genomic context of distinct continuously spaced sets of disease-linked SNPs and mRNAs of relevant protein-coding genes by analyzing two sets of SNPs which are located within continuous genomic regions associated with individual protein-coding genetic loci (NLRP1 and STAT4) and are likely to exhibit common profiles of transcriptional activity.

Sequence Homology Profiling of Disease-Linked SNPs Associated with NLRP1 and STAT4 Loci Identifies Allele-Specific MirMaps with Distinct Targeting Potentials Against mRNAs of the Importin Genes

Analysis of transcriptional regulation of genetic loci harboring disease-linked SNPs began with a disease phenocode analysis two sets of SNPs were selected for, which are derived from continuous genomic regions associated with individual protein-coding genes and are likely to exhibit common profiles of transcriptional activity. The results focus on eight SNPs associated with the NLRP1 (NALP1) loci, including six disease-linked SNPs of the NLRP1 (NALP1) promoter region, strong association of which with vitiligo and multiple associated autoimmune disorders was recently reported. The analysis included two major independent association signals which are represented by the rs6502867 and rs4790797 markers as well as SNPs located within a 64.7 kb linkage disequilibrium block tagged by the rs12150220 and six NLRP1 promoter-region SNPs (rs2670660, rs878329, rs7223628, rs8182352, rs4790796, and rs4790797). The SNPs rs878329, rs7223628, rs8182352, rs4790796 are in almost perfect linkage disequilibrium with rs4790797, and all 5 of these SNPs are located within a continuous genomic region which span only 2.1 kb. Disease phenocode analysis of NLRP1-associated SNPs identifies an SNP-guided MirMap comprising 7 SNPs and 27 microRNAs, 16 of which are represented in the TargetScan database. (See Table 13).

TABLE 13
Overlapping network of microRNAs with targeting potentials
against mRNAs of the KPNA1, KPNA6, NLRP1, NLRP3 and
STAT4 genes

| GENE | KPNA1 | KPNA6 | NLRP1 | NLRP3 | STAT4 |
|---|---|---|---|---|---|
| KPNA1 | 192 | 1.4E−22 | 0.00013 | 0.0002 | 0.01568 |
| KPNA6 | 107 | 191 | 7.1E−06 | 3E−05 | 0.00149 |
| NLRP1 | 21 | 23 | 37 | 0.0324 | 0.37463 |
| NLRP3 | 20 | 22 | 5 | 37 | 0.02196 |
| STAT4 | 11 | 13 | 1 | 4 | 22 |

Numbers of human KPNA1-, KPNA6-, NLRP1-, NLRP3- and STAT4-targeting microRNAs (in bold) were identified using TargetScan database. p values were calculated using hypergeometric distribution tests. They represent the estimates of the likelihood of obtaining score values by chance and take into account the numbers of all screened for homology microRNAs and the number of microRNAs which are predicted to target a given target gene.

Sixty nine percent of identified microRNAs shown in Table 13 have the potential to target mRNA sequences derived from importin genes and 6 of 7 SNPs (86%) have sequence homology to the importin mRNA-targeting microRNAs.

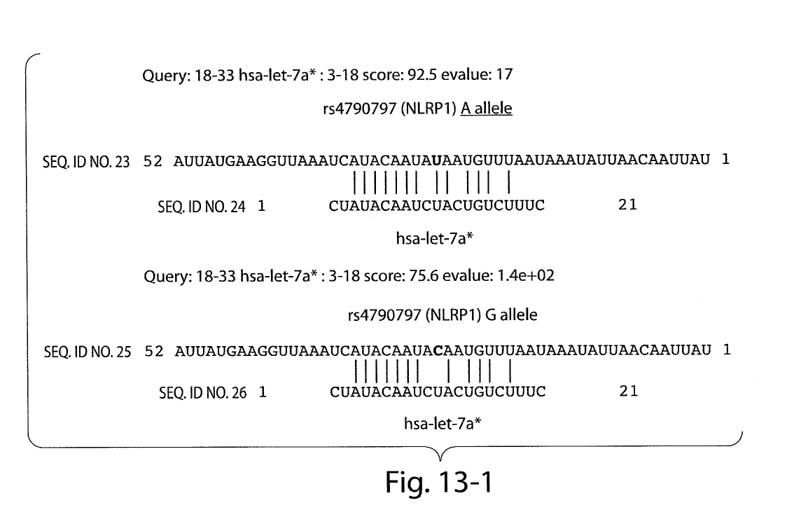

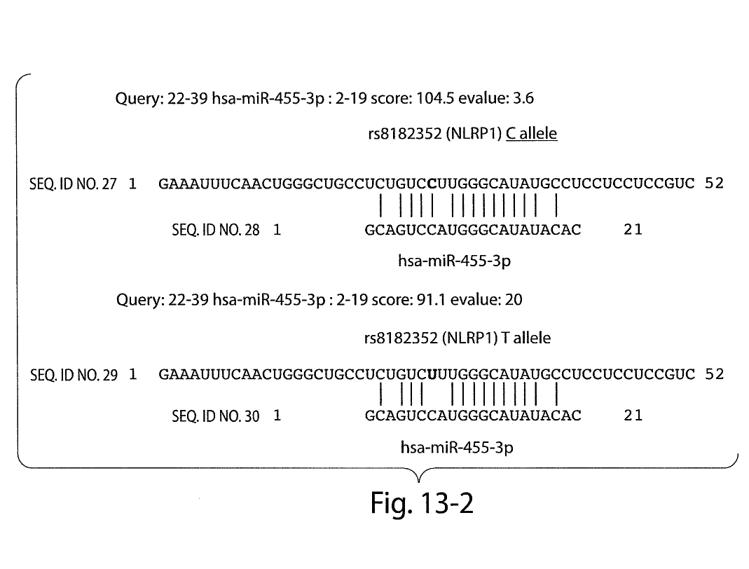

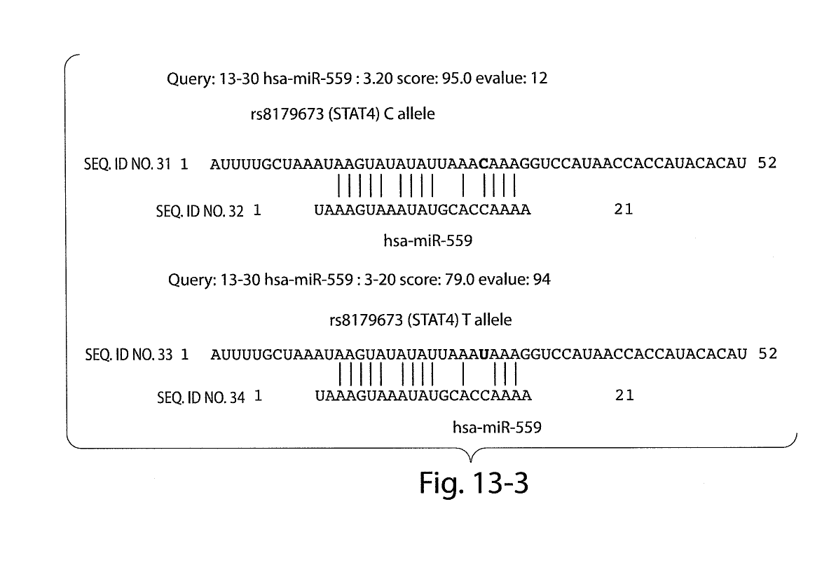

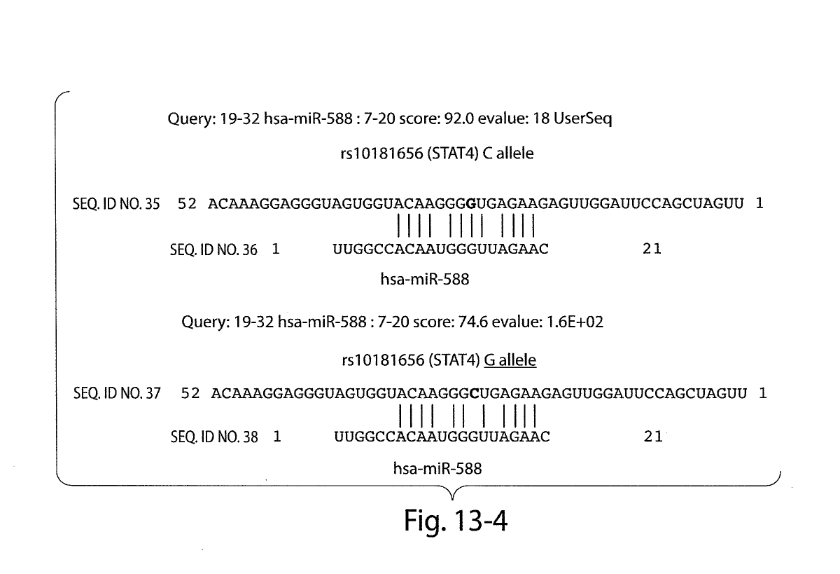

The effects of allele-specific changes of disease-linked SNPs/microRNAs homology profiles on microRNA-targeting potency against mRNAs encoded by the KPNA1 and KPNA6 genes was also explored. According to a disease phenocode hypothesis, decreased sequence homology scores and increased e-values of disease-linked SNPs to KPNA1-targeting microRNAs reflected diminished capacity of SNP sequence-bearing transcripts to interfere with bioactivity/biogenesis of homologous microRNAs. Thus, an intracellular context favoring higher KPNA1-targeting potency of multiple microRNAs was facilitated, which increased the probability of the KPNA1-deficient phenotypes. Conversely, increased sequence homology scores and decreased e-values of disease-linked SNPs to KPNA1-targeting microRNAs reflected the augmented capacity of SNP sequence-bearing transcripts to interfere with bioactivity/biogenesis of homologous microRNAs. This scenario would facilitate an intracellular context favoring lower KPNA1-targeting potency of multiple microRNAs thus increasing the probability of the KPNA1-overexpression phenotypes. Representative examples of primary sequence alignments illustrating allele-specific changes of sequence homology profiles of identified in this study disease-linked SNP/microRNA pairs which are associated with NLRP1 and STAT4 loci are shown in FIG. 13.

Targeting potential of individual microRNAs against specific mRNA targets is estimated using the values of the context scores as defined by the TargetScan algorithm, according to which the lower values of the context scores reflect the higher mRNA targeting potency of a microRNA. To calculate formal numerical values reflecting the mRNA-targeting potency of a given microRNA within the allele-specific context of a homologous disease-linked SNP, the microRNA/mRNA pair-specific context score was multiplied by the allele-specific microRNA/SNP sequence homology e-value, so the relationships between the lower values of the calculated allele-specific microRNA/mRNA context scores and higher mRNA targeting potency of a given microRNA would be maintained. Cumulative disease mRNA-targeting scores were obtained by adding individual mRNA-targeting scores calculated for each microRNAs within the context of high-risk SNP alleles. Conversely, cumulative control mRNA-targeting scores were obtained by adding individual mRNA-targeting scores calculated for each microRNAs within the context of low-risk SNP alleles.

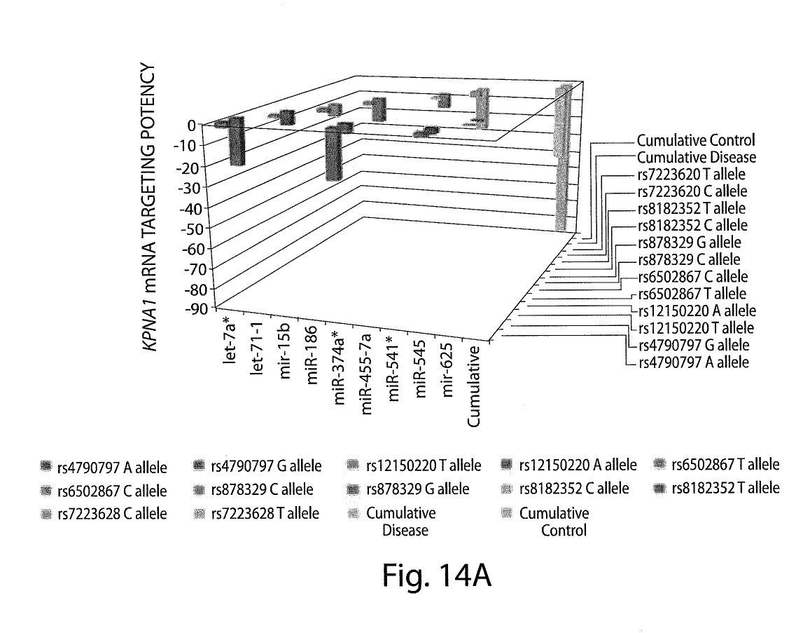

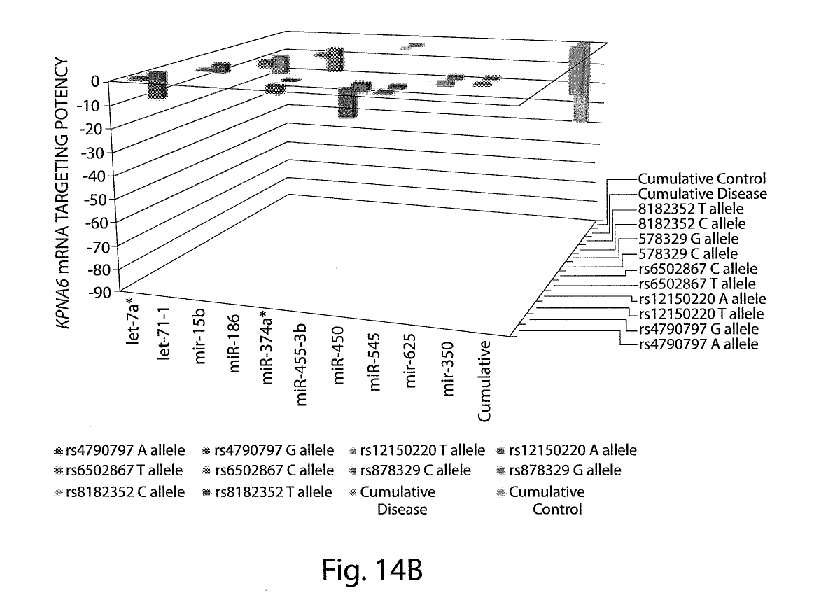

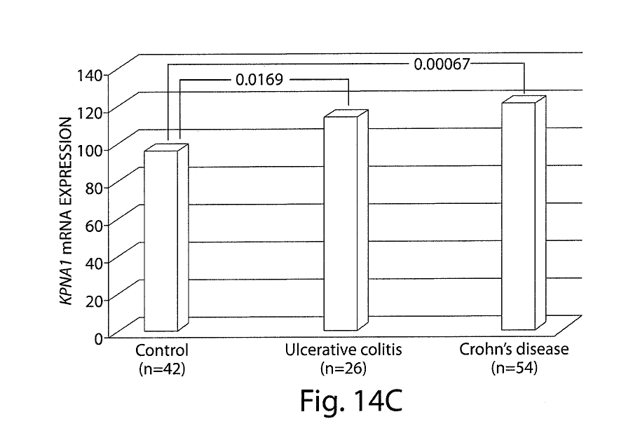

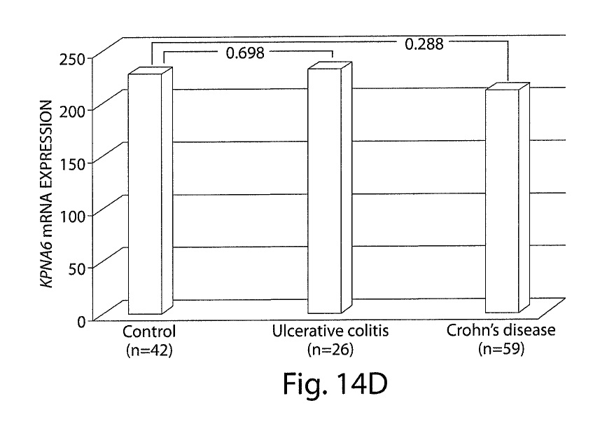

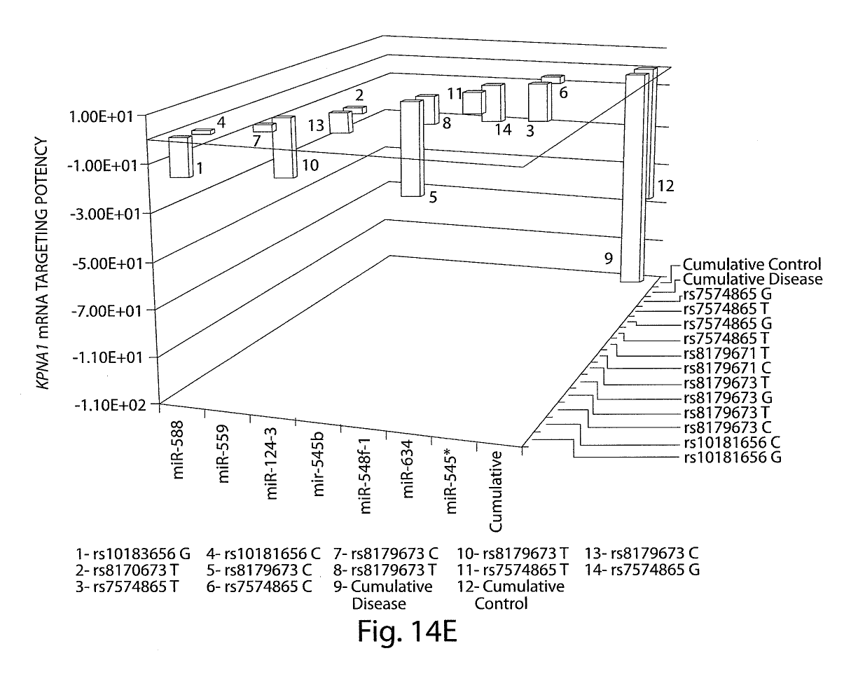

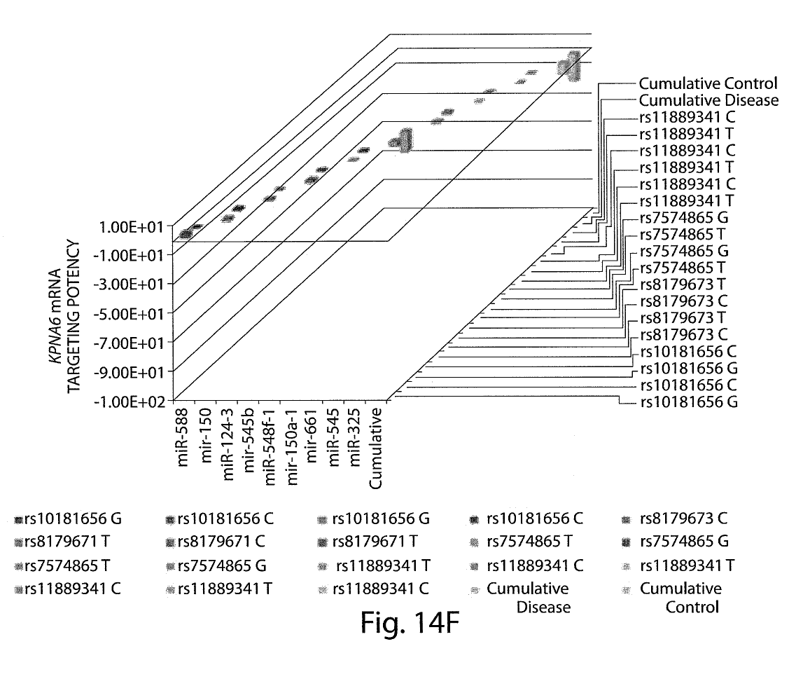

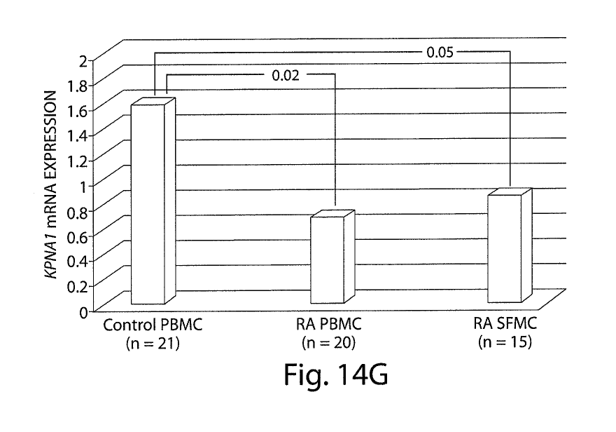

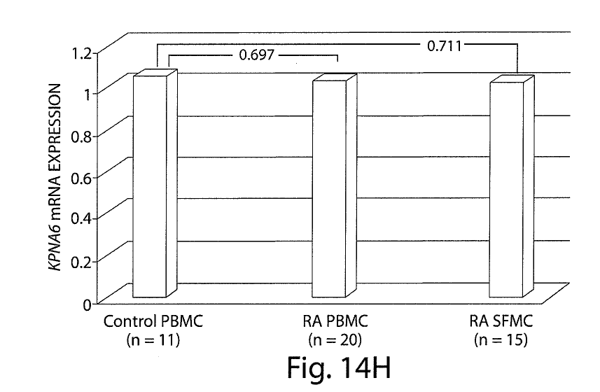

Allele-specific maps of microRNA-targeting potency against mRNA of the KPNA1 and KPNA6 genes demonstrate that, while the predicted targeting potentials are diminished for both genes in a disease state context, the magnitude of changes for KPNA1 mRNA targeting appears 3-fold greater compared to the KPNA6 mRNA targeting (FIGS. 14A, 14B). Cumulative targeting scores for a disease state compared to control are higher by 48.7 and 16.2 relative targeting potency units (RTPUs) for KPNA1 and KPNA6 mRNAs, respectively, suggesting that the expression levels of the KPNA1 mRNA should be increased relatively to the KPNA6 mRNA expression in patients with autoimmune disorders. Microarray analysis reveals increased expression of the KPNA1 mRNA in peripheral blood mononuclear cells (PBMC) of patients with UC and CD, whereas expression of a closely related importin alpha gene, KPNA6, is not altered (FIGS. 14C, 14D).

Four SNPs linked with increased risk of rheumatoid arthritis (RA) (rs10181656; rs8179673; rs7574865; rs11889341) which are located within a continuous genomic region associated with the STAT4 gene were analyzed. Allele-specific maps of microRNA-targeting potency reveal increased predicted cumulative targeting potentials for KPNA1 mRNAs in a disease state context, whereas cumulative targeting potentials for KPNA6 mRNAs seems lower for high-risk allele's context compared to controls. (See FIGS. 14E, 14F). The magnitude of changes for the predicted KPNA1 mRNA targeting appears 3.2-fold greater compared to the KPNA6 mRNA targeting. (See FIGS. 14E, 14F). Cumulative targeting scores for a disease state compared to controls are lower by 37.8 RTPUs (relative targeting potency unit as defined in the Example 1 herein for the mRNA-targeting potential of individual microRNAs and cumulative mRNA-targeting scores for disease states and control subjects) and higher by 11.7 RTPUs for KPNA1 and KPNA6 mRNAs, respectively, thereby suggesting that the expression levels of the KPNA1 mRNA should be decreased relatively to the KPNA6 mRNA expression in patients with RA. Microarray analysis demonstrates decreased expression of the KPNA1 mRNA in mononuclear cells of patients with RA, whereas expression of a closely related importin alpha gene, KPNA6, is not altered. (See FIGS. 14G, 14H). Thus, in contrast to disease-linked SNPs associated with the NLRP1 gene, a disease state context of the STAT4-associated SNPs seems to reflect a regulatory balance favoring decreased expression of the KPNA1 gene.

Disease Phenocode Analysis of the MicroRNAs Homologous to the NLRP1 Promoter Region SNP rs2670660

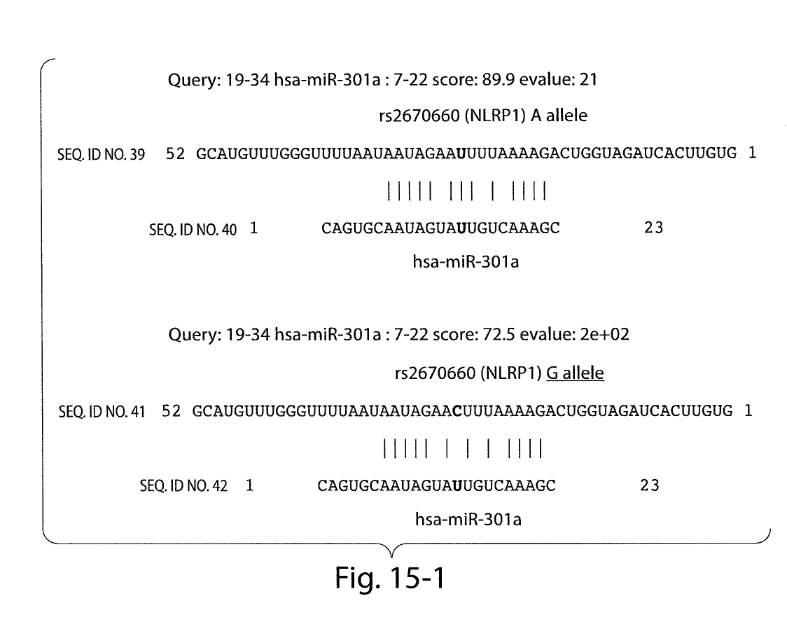

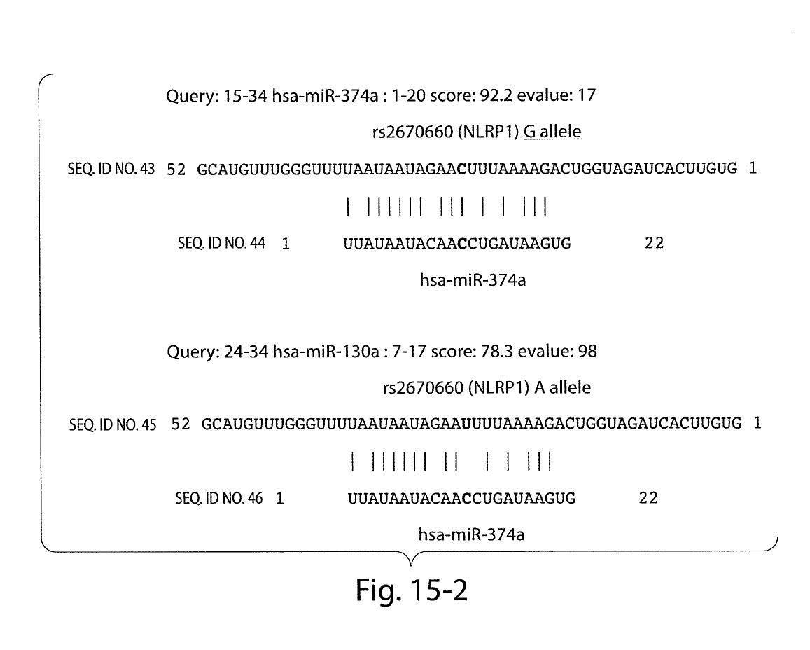

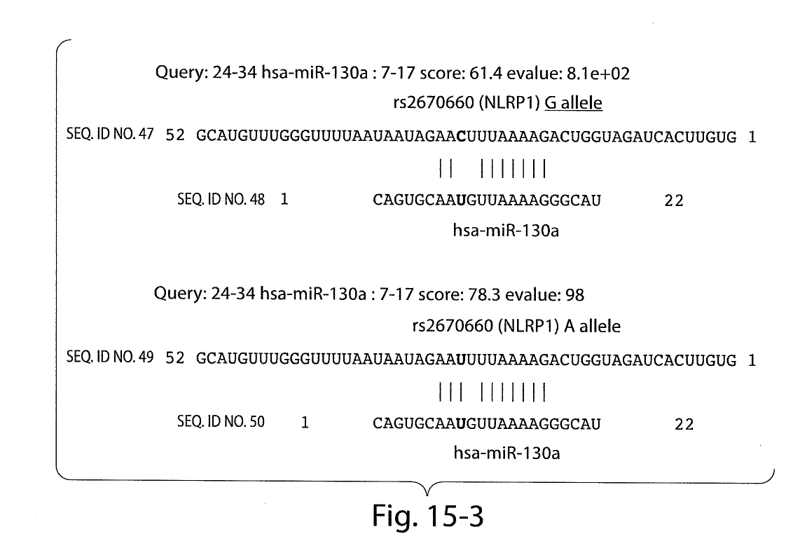

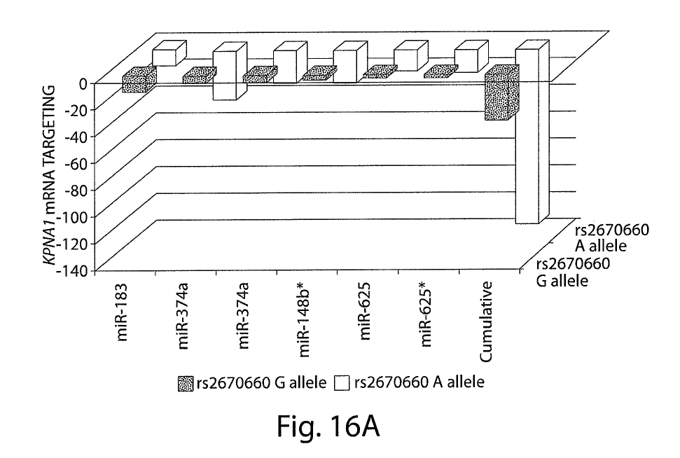

One of the disease-linked SNPs associated with the promoter region of the NLRP1 gene, rs2670660, is of particular interest. It has been noted that rs2670660 is located within a genomic segment which is highly evolutionary conserved in the human, chimpanzee, macaque, bush baby, cow, mouse, and rat. (See Jin Y., et al., NALP1 in vitiligo-associated multiple autoimmune disease. N. Engl. Med. 356:1216-1225 (2007)). Furthermore, rs2670660 variants appear to alter the predicted transcription factor binding sites for HMGA1 and MYB, which is consistent with the postulated regulatory role of this SNP. Sequence homology profiling identifies 7 microRNAs homologous to the rs2670660 (e value cut-off 50), five of which are listed in the TargetScan database. All five rs2670660-homologous microRNAs are predicted to target mRNAs encoded by importin genes (Table 14), indicating that importin mRNA-targeting is a common feature of this set of microRNAs. Examples of allele-associated changes of the rs2670660 sequence homology to the hsa-miR-301a, hsa-miR-374a, and hsa-miR130a are shown in FIG. 15.

TABLE 14
Targeting of mRNAs of principal components of NIP and inflammasomes pathways by
microRNAs homologous to the disease-associated SNPs of the NLRP1 promoter region

| mRNA/ | | | | | | | |
|---|---|---|---|---|---|---|---|
| microRNA | KPNA1 | KPNA2 | KPNA3 | KPNA4 | KPNA5 | KPNA6 | KPNB1 |
| let-7 | let-7 | | | let-7 | | let-7 | |
| miR-141* | | | miR-141 | miR-141 | | | |
| mir-150 | | | | | | mir-150 | |
| mir-15a | mir-15 | | mir-15 | mir-15 | | mir-15 | |
| miR-185* | | | | | | | |
| miR-186 | miR-186 | | miR-186 | | | miR-186 | |
| mir-337 | | | | | | | |
| miR-374a* | miR-374 | | | | | miR-374 | |
| mir-422a | | | | | | | |
| mir-450b | | | | | | mir-450 | |
| miR-455-3p | miR-455 | | miR-455 | | | miR-455 | |
| mir-521-2 | | | | | | | |
| miR-541* | miR-541 | | | | | | |
| miR-545 | miR-545 | | | miR-545 | | miR-545 | miR-545 |
| miR-553 | | | | | | | |
| mir-625 | mir-625 | | | mir-625 | | mir-625 | |
| Score | 8 | 0 | 4 | 5 | 0 | 9 | 1 |
| P value | 0.0356 | 0.4929 | 0.1252 | 0.04248 | 0.5668 | 0.00704 | 0.3391 |

| mRNA/ | | | | | | |
|---|---|---|---|---|---|---|
| microRNA | KPNB2 | KNPB3 | NLRP1 | NOD2 | NLRP3 | PYCARD |
| let-7 | | let-7 | | | | |
| miR-141* | miR-141 | miR-141 | | | miR-141 | |
| mir-150 | | mir-150 | | | | |
| mir-15a | mir-15 | | | | | |
| miR-185* | | | | | | |
| miR-186 | miR-186 | miR-186 | | | miR-186 | |
| mir-337 | | | mir-337 | | | |
| miR-374a* | miR-374 | miR-374 | | | | |
| mir-422a | | | | | | |
| mir-450b | mir-450 | | | | | |
| miR-455-3p | miR-455 | | | | | |
| mir-521-2 | | | | | | |
| miR-541* | | | | | | |
| miR-545 | miR-545 | | | | | |
| miR-553 | | | | | | |
| mir-625 | mir-625 | | | | | |
| Score | 8 | 5 | 1 | 0 | 2 | 0 |
| P value | 0.0112 | 0.0914 | 0.3778 | 0.2993 | 0.1511 | 0.8942 |

Human importin- and inflammasome-targeting microRNAs were identified using TargetScan database. p values were calculated using hypergeometric distribution tests. They represent the estimates of the likelihood of obtaining score values by chance and take into account the numbers of all screened for homology microRNAs and the number of microRNAs which are predicted to target a given target gene. For mir-1243 and mir-1245: No data in the TargetScan database.

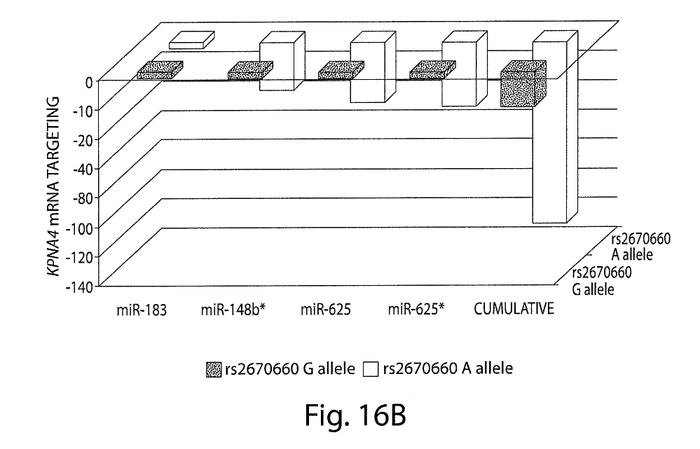

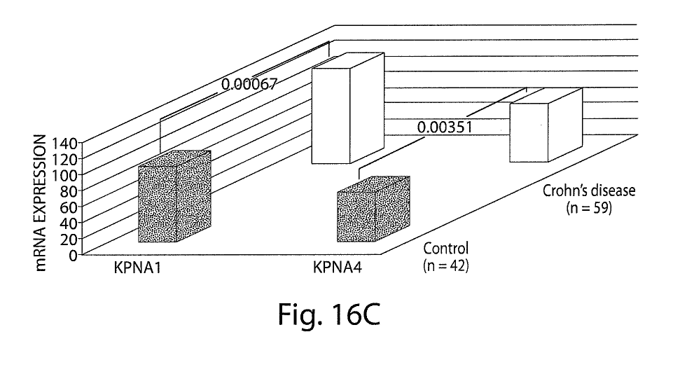

Allele-specific maps of microRNA-targeting potency against KPNA1 and KPNA4 mRNAs demonstrate that the predicted targeting potentials are decreased for both genes in a disease-state context and the magnitude of changes of mRNA targeting potencies is similar for KPNA1 and KPNA4 genes. (See FIGS. 16A, 16B). Expression of both KPNA1 and KPNA4 genes is likely to be elevated in patients with autoimmune/autoinflammatory disorders. Consistent with this analysis, gene expression profiling experiments reveal increased levels of KPNA1 (p=0.00067) and KPNA4 (p 0.00351) mRNAs in PBMC of CD patients. (See FIG. 16C).

Whether changes of the microRNA targeting potency within the rs2670660 risk allele context would manifest a similar pattern of association with mRNA expression of a broader set of genes was also explored. In particular, predicted mRNA targets for hsa-miR-130/301 and hsa-miR-374 (see FIG. 15) were examined. High-risk allele sequence of the rs2670660 has increased homology to the miR-374 microRNA, which is reflected by 5.8-fold lower e value of the high-risk variant compared to the low-risk allele. (See FIG. 15). In contrast, rs2670660 high-risk allele manifests a decreased homology to the miR-301 and miR-130 microRNAs, which is reflected by the 9.5-fold and 5.8-fold higher e values of the high-risk variants compared to the low-risk alleles, respectively. (See FIG. 15). microRNA-interfering potentials of the rs2670660 high-risk allele would be higher with respect to the miR-374 and lower with respect to the miR-301 and miR-130 microRNAs.

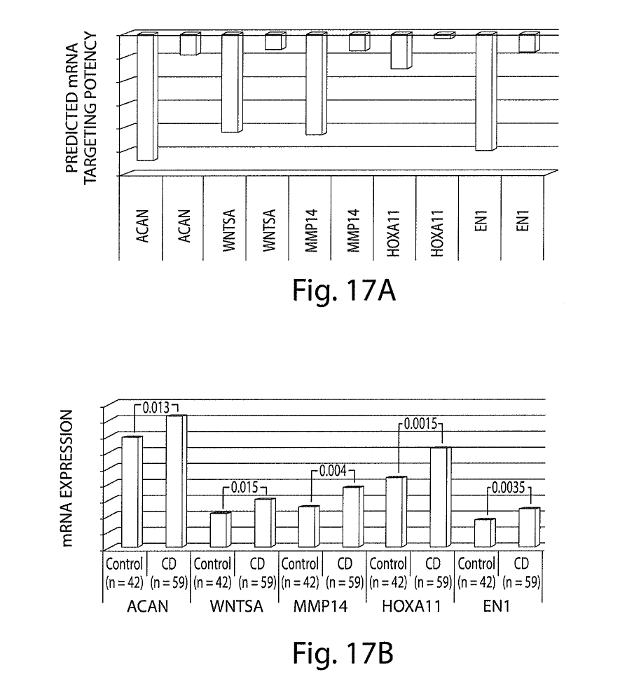

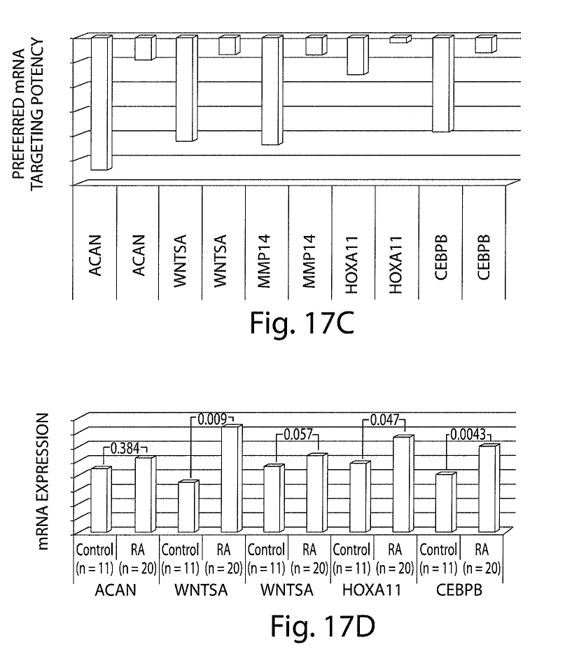

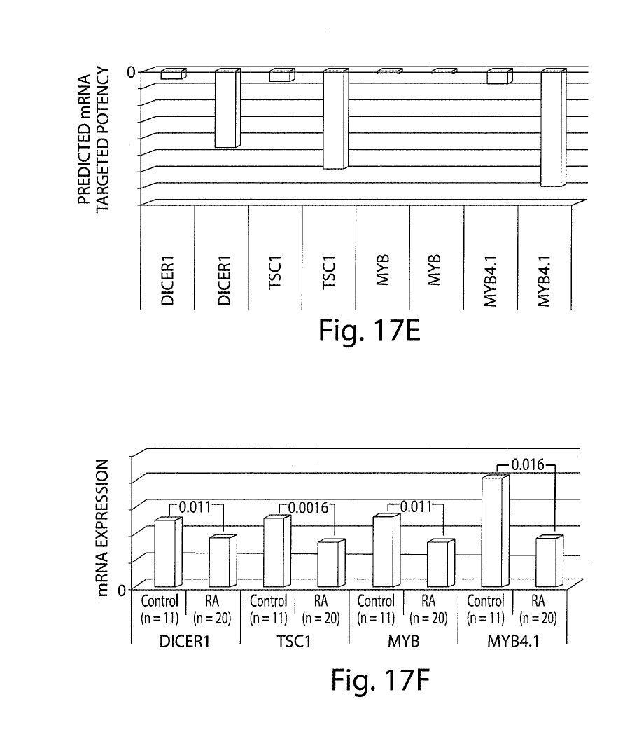

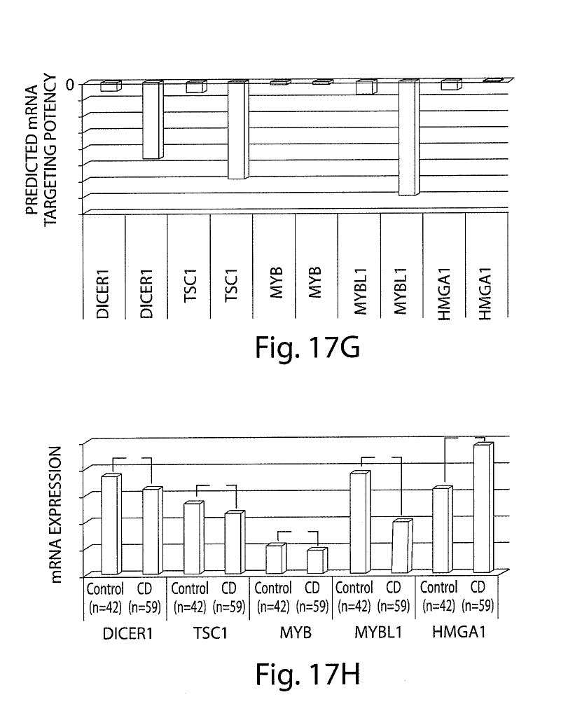

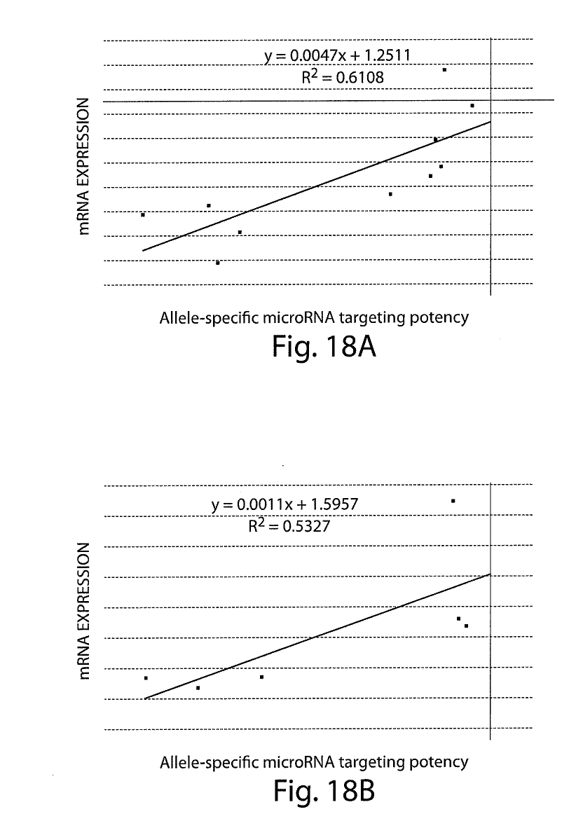

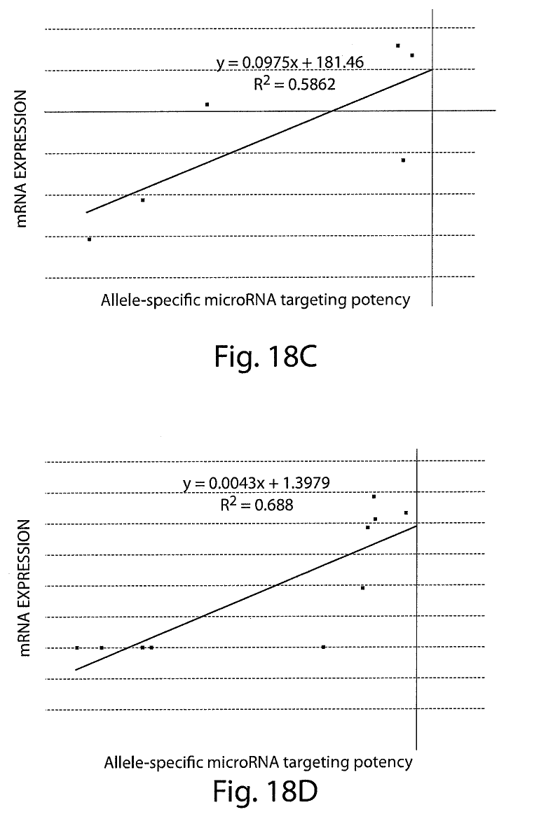

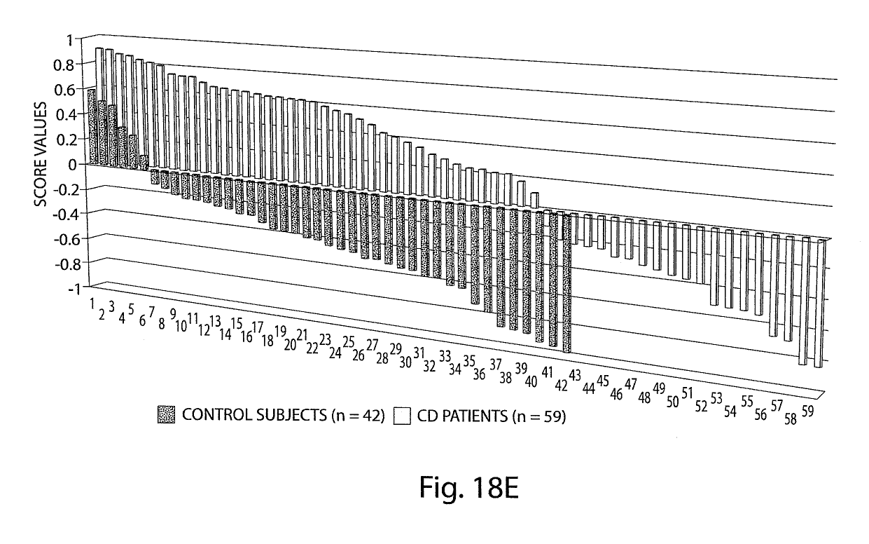

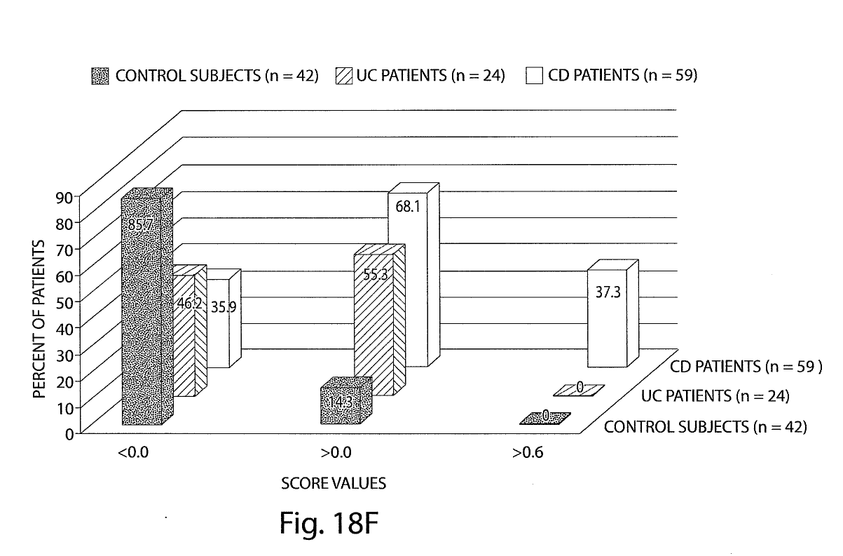

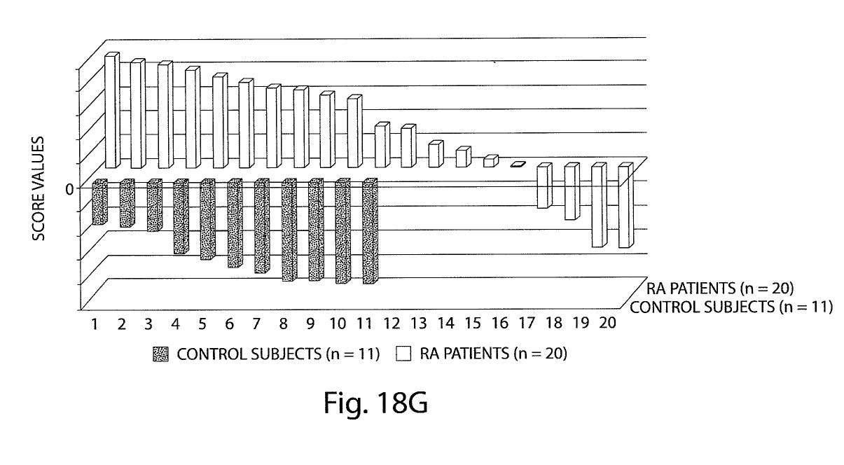

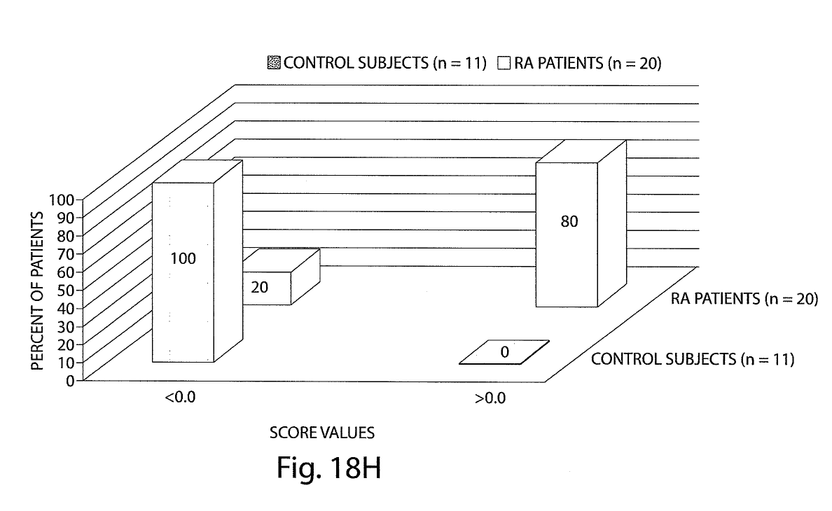

Correspondingly, mRNA-targeting potency of the miR-374 is predicted to be lower within a disease state context, whereas mRNA-targeting potency of the miR-301 and miR-130 is predicted to be higher within a disease state context. The estimates of the mRNA-targeting potency within disease state and control contexts for sets of genes mRNAs were calculated of which are potential targets for miR-374 and miR-130/301 and show distinct expression in the PBMC of CD and RA patients compared to control subjects. (See FIG. 17). In agreement with the predicted decreased mRNA-targeting potency of the miR-374 microRNA (see FIGS. 17A, 17B), the mRNA expression levels of the ACAN, WNT5a, MMP14, and HOXA11 target genes are higher in PBMC of both CD and RA patients. (See FIGS. 17B, 17D). Conversely, in agreement with the predicted increased mRNA-targeting potency of the miR-130 and miR-301 microRNAs (see FIGS. 17E, 17G), the mRNA expression levels of the DICER1, TSC1, and MYBL1 target genes are lower in PBMC of both CD and RA patients. (See FIGS. 17F, 17H). The corresponding Pearson correlation coefficients between the allele-specific microRNA/mRNA targeting estimates and mRNA expression values were calculated, and found that correlations seem statistically significant in all instances: Pearson coefficients r=0.83 (p=0.003) and r=0.78 (p=0.0076) for miR-374 targets in CD and RA patients, respectively; Pearson coefficients r=0.85 (p=0.0329) and r=0.98 (p=0.0006) for miR-130/301 targets in CD and RA patients, respectively. Graphical illustrations of these observations are presented in FIG. 18.

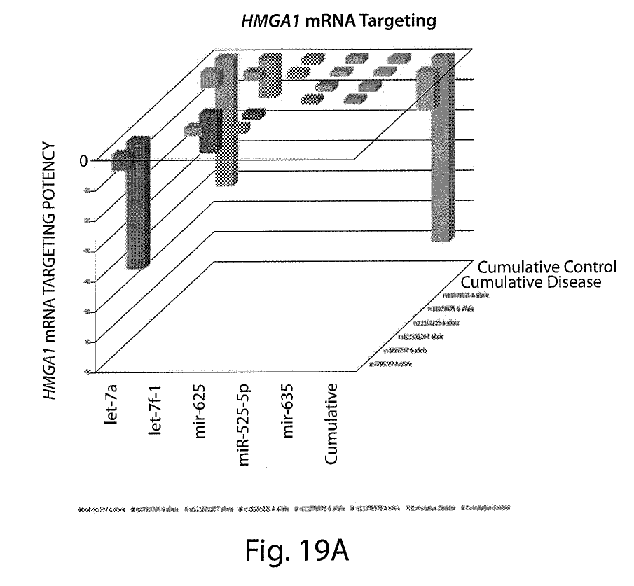

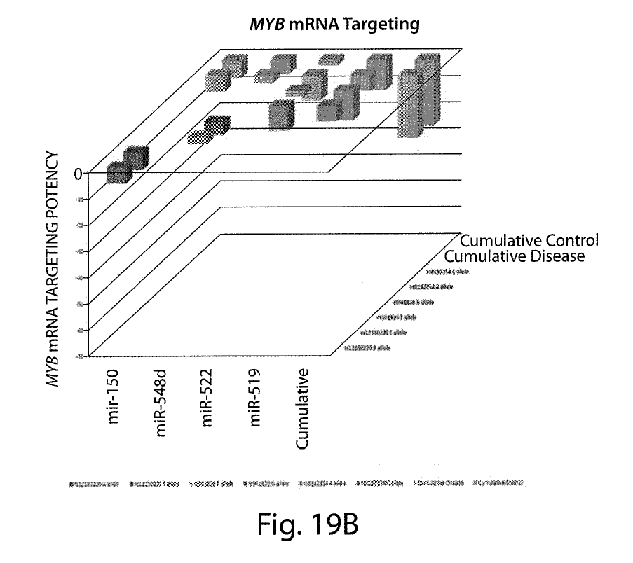

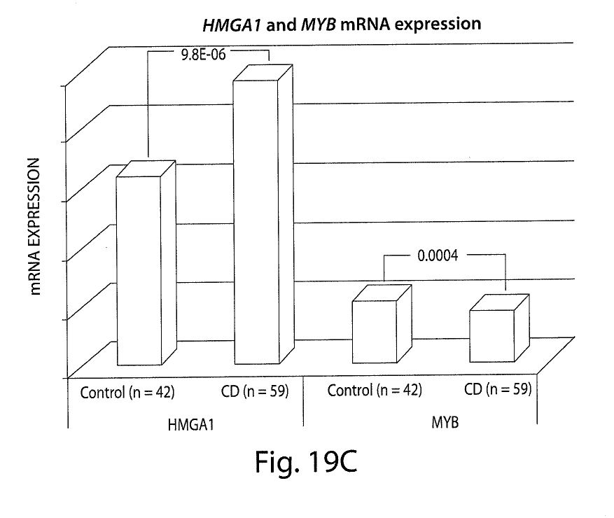

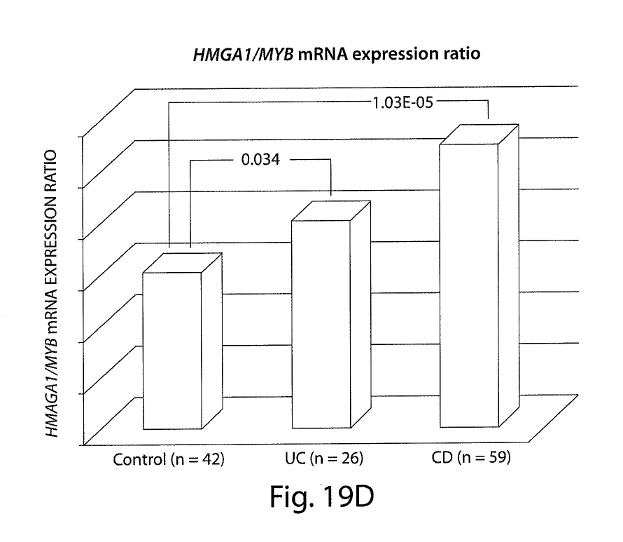

These results indicated that the rs2670660 sequence may represent a transcription binding site and suggested that rs2670660 variants may alter the predicted binding motifs for HMGA1 and MYB transcription factors. The allele-specific targeting potency was compared against HMGA1 and MYB mRNAs of microRNAs homologous to the rs2670660 SNP. Comparisons of HMGA1- and MYB-targeting allele-specific MirMaps (see FIGS. 19A, 19B) reveal that cumulative HMGA1 mRNA-targeting potency is 4.8-fold lower in a risk-allele context (−12.7 RTPUs versus −60.7 RTPUs in disease and control states, respectively), whereas MYB mRNA targeting appears not altered (−23.8 RTPUs versus −24.9 RTPUs in disease and control states, respectively). These data suggest that the intracellular context associated with NLRP1 gene-driven autoimmune/autoinflammatory phenotypes would facilitate regulatory balance favoring increased level of expression of the HMGA1 mRNA. (See FIGS. 19A, 19B). Gene expression profiling experiments demonstrate that the expression level of the HMGA1 mRNA is higher and the ratio of HMGA1/MYB mRNAs is elevated in PBMC of CD patients (See FIGS. 19C, 19D).

Disease Phenocode Analysis Identifies Common Profiles of the SNP Risk Allele-Associated Changes of MicroRNA Targeting Potency and mRNA Expression of the Principal Components of Inflammasome Pathways in CD and RA Patients

Despite mounting experimental evidence implicating components of inflammasome pathways and innate immune system in the pathogenesis of multiple autoimmune and autoinflammatory disorders (see Jin Y., et al., NALP1 in vitiligo-associated multiple autoimmune disease. N. Engl. Med. 356:1216-1225 (2007)), the underlying molecular causes of the inflammasome malfunction in human diseases remain obscure.

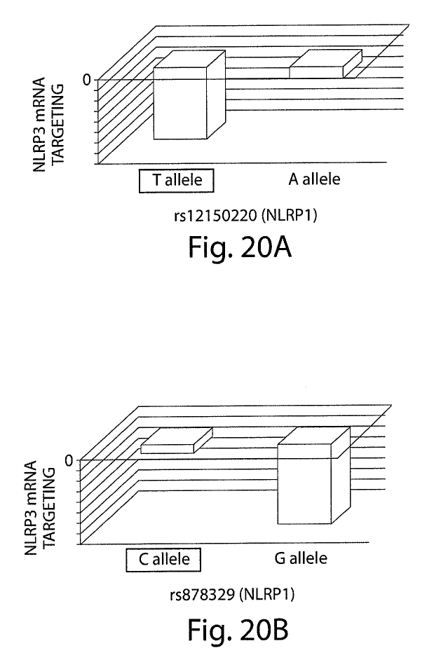

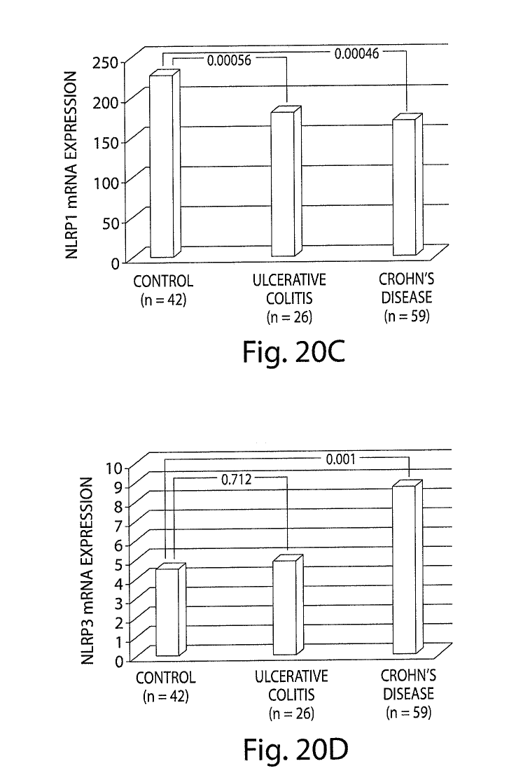

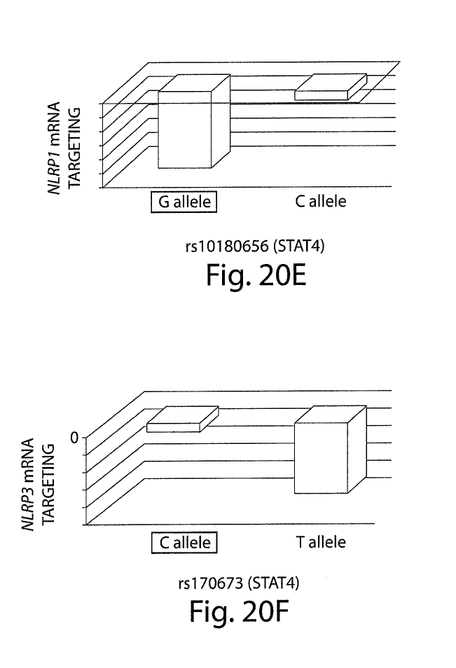

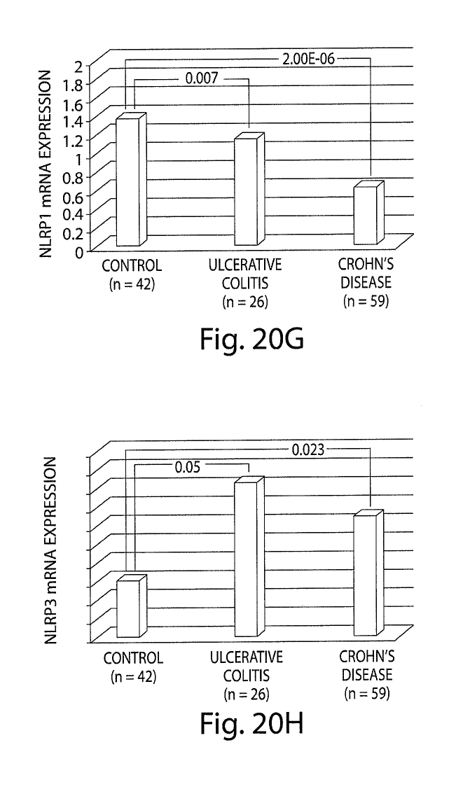

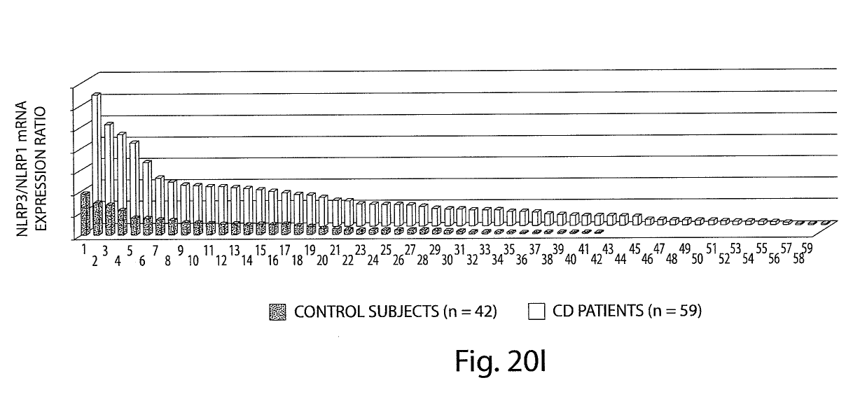

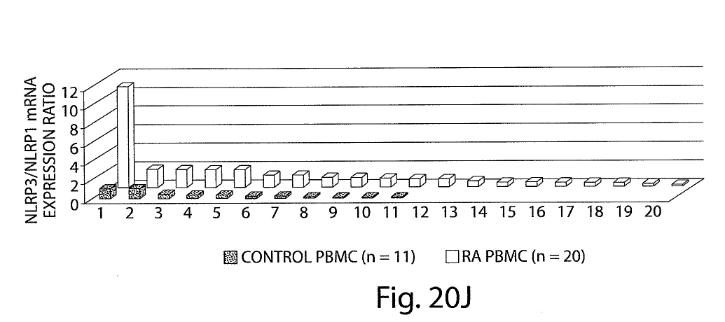

Analysis of microRNA targeting potency against mRNAs of principal components of inflammasomes pathways by microRNAs homologous to the disease-linked SNPs associated with the NLRP1 and STAT4 genes was performed. Four microRNAs with distinct patterns of low-risk allele- and high-risk allele-associated changes of mRNA targeting were identified. (See FIGS. 20A, 20B, 20E, 20F). Both NLRP1 mRNA-targeting microRNAs (a sequence homologue of NLRP1 SNP rs12150220, hsa-mir-337; and a sequence homologue of STAT4 SNP rs10181656, hsa-miR-588) manifest markedly higher mRNA targeting potency in a disease state context having targeting scores lower by 5.6-fold and 8.9-fold for hsa-mir-337 and hsa-mir-588, respectively. (See FIGS. 20A and 20E). These data suggest that mRNA expression of the NLRP1 gene should be decreased in both CD and RA patients. (See FIGS. 20A and 20E). Conversely, both NLRP3 mRNA-targeting microRNAs (a sequence homologue of NLRP1 SNP rs878329, hsa-miR-186; and a sequence homologue of STAT4 SNP rs8179673, hsa-miR-559) manifest markedly lower mRNA targeting potency in a disease state context having targeting scores higher by 7.75-fold and 7.83-fold for hsa-miR-186 and hsa-miR-559, respectively (See FIGS. 20B and 20F). This implies that mRNA expression of the NLRP3 gene should be elevated in both CD and RA patients. (See FIGS. 20B and 20F). Microarray analysis reveals consistent statistically significant gene expression changes in mononuclear cells of both CD (see FIGS. 20C and 20D) and RA. (See FIGS. 20C and 20D) patients, constituting a decreased NLRP1 mRNA expression and an increased NLRP3 mRNA expression (See, FIGS. 20C, 20D, 20G, 20H).

Gene Expression Signatures of miR-374 and miR-130/301 mRNA Targets Reflect Activated States of the Disease-Linked SNP/MicroRNA/mRNA Axis in a Majority of CD and RA Patients

Microarray analysis demonstrates altered expression in PBMC of CD and RA patients of multiple genes mRNAs of which are potential targets of microRNAs homologous to disease-linked SNPs. However, comparisons of the average gene expression values between disease cohorts and control groups do not provide information regarding the prevalence within patient populations of the associations between gene expression alterations and disease phenotypes. A gene expression signature approach was applied to estimate how frequently the postulated functional axis of disease-linked SNPs/microRNAs/mRNAs is engaged in individual CD and RA patients. Gene signatures comprising of miR-374 and miR-130/301 mRNA targets were designed and ten-gene signature score values were calculated for individual patients and control subjects using the previously described Pearson correlation method. The signature analysis results demonstrate that 39 of 59 (66%) CD patients and 16 of 20 (80%) RA patients have ten-gene signature score values>0.0. (See FIGS. 18E-H). In contrast, 47 of 53 (89%) control subjects manifest ten-gene signature score values<0.0. (See FIGS. 18E-H). Notably, a significant fraction of both CD and RA patients [22 of 59 (37%) CD patients and 9 of 20 (45%) RA patients] and none of control subjects have ten-gene signature score values>0.6. The data suggests that disease-linked SNP/microRNA/mRNA axis is activated in a majority of CD and RA patients (See FIGS. 18E-H). Independent analysis of the frequency of alterations of NLRP1 and NLRP3 mRNA expression in PBMC of CD and RA patients appears to support this conclusion. (See FIGS. 201 and 20J).

Genome-wide sequence homology profiling analysis identifies SNP-guided MirMaps which reveal common features of disease-linked SNPs and microRNAs of a consensus disease phenocode. Nearly all consensus microRNAs (15 of 17; 88%) have potential protein-coding mRNA targets among the principal components of the nuclear import pathway (NIP) and/or the inflammasome/innate immunity pathways. Many microRNAs of the genome-wide consensus set have the apparent propensity to target mRNAs of the selected inflammasome-related genes, namely NLRP1, NLRP3, NLRP4, NLRP1 and NLRP3 genes are the principal components of the corresponding NLRP1- and NLRP3-inflammasomes and NLRP4 protein modulates NF-kappa B induction by inflammatory cytokines, in particular, by the interleukin-1-beta, production of which is increased as a consequence of inflammasome activation. All 81 disease-linked SNPs of the consensus set manifest sequence homology to microRNAs that are predicted to target mRNAs of importin genes, which indicates that sequence homology to the importin-targeting microRNAs is a common structural feature of many SNPs associated with multiple major human diseases. microRNAs with targeting potentials against mRNAs of the KPNA1, KPNA6, NLRP1, STAT4, and NLRP3 genes appear to form a statistically valid overlapping network (see Table 13), underscoring the presence of common structural features in the 3′ UTR regions of these genes.

As noted, the disease phenocode hypothesis postulates that in trans effects on phenotypes of SNP sequence-bearing RNAs would depend on level of expression of SNP-harboring genetic loci, implying that transcriptionally co-regulated SNP-sequence-bearing RNAs are more likely to exert a cumulative effect on phenotypes. Compelling experimental evidence generated by tiling array expression profiling studies indicates that expression of non protein-coding RNAs are coincidental with corresponding protein-coding genetic loci, suggesting a common mechanism of transcriptional regulation. Therefore, DNA segments within continuous genomic regions associated with individual protein-coding genetic loci are likely to exhibit common profiles of transcriptional activity. The results presented herein support the validity of utilizing a disease phenocode concept for the genomic contexts of distinct continuously spaced sets of disease-linked SNPs and mRNAs of relevant protein-coding genes by analyzing two sets of SNPs, which are located within continuous genomic regions associated with the NLRP1 and STAT4 genes.

NLPRP1- and STAT4-associated disease-linked SNPs have sequence-defined features which are recapitulate common phenotype-affecting features of genome-wide disease-linked SNPs, thereby suggesting that NLRP1 and STAT4 genetic loci may constitute “master” disease genes. Similar to microRNAs homologous to genome-wide disease-linked SNPs, 15 of 19 (79%) of microRNAs homologous to NLRP1-associated disease-linked SNPs have potential mRNA targets among principal components of nuclear import and/or inflammasome/innate immunity pathways (see Tables 14 and 15).

TABLE 15
Importin mRNA-targeting map of the microRNA homologous to the re2670660 of the
NLRP1 promoter region

| mRNA/microRNA | KPNA1 | KPNA2 | KPNA3 | KPNA4 | KPNA5 | KPNA6 | KPNB1 | KPNB2 | KNPB3 |
|---|---|---|---|---|---|---|---|---|---|
| miR-148 | miR-148 | | | miR-148 | | | miR-148 | miR-148 | miR-148 |
| miR-183 | miR-183 | | | miR-183 | | | | miR-183 | |
| miR-301 | | | | | | | | miR-301 | |
| miR-374 | miR-374 | | | | | miR-374 | | miR-374 | miR-374 |
| miR-625 | miR-625 | | | miR-625 | | miR-625 | | miR-625 | |
| Score, microRNA | 4 | 0 | 0 | 3 | 0 | 2 | 1 | 5 | 2 |
| p values | 0.022426 | 0.791357 | 0.427395 | 0.023357 | 0.828738 | 0.294375 | 0.334937 | 0.000853 | 0.193689 |

microRNA scores represent the number of microRNAs with potential to target mRNAs encoded by a given importin gene. Human importin-targeting microRNAs were identified using TargetScan database. p values were calculated using hypergeometric distribution tests. They represent the estimates of the likelihood of obtaining score values by chance and take into account the numbers of all screened for homology microRNAs and the number of microRNAs which are predicted to target a given importin gene. For mir-1243 and mir-1245: No data in the TargetScan database.

Furthermore, 7 of 8 (88%) NLRP1-associated disease-linked SNPs manifest sequence homology to microRNAs which have targeting potentials against mRNAs encoded by the importin genes. Both genome-wide SNP variations and SNP polymorphisms associated with “master” disease genes may cause similar genetically-defined malfunctions of the NIP and inflammasome pathways, which are likely to contribute to pathogenesis of multiple common human disorders.

Theoretical estimates of cumulative targeting effects of microRNAs on mRNAs within distinct allelic contexts of disease-linked SNPs are in agreement with experimentally-defined gene expression phenotypes associated with human genotypes of CD and RA populations. Microarray analysis of peripheral blood mononuclear cells (PBMC) demonstrates statistically significant KPNA1-, NLRP1-, and NLRP3-gene expression phenotypes associated with human genotypes of CD, HD, and RA populations. Gene expression profiling of PBMC from patients treated with chloroquine reveals a reversal of disease-linked KPNA1-, NLRP1-, and NLRP3-gene expression phenotypes, implying that chloroquine could serve as a readily available drug for targeted correction of identified aberrations. Taken together, these results set up an experimental framework for development of PBMC-based tests, which may be clinically useful for targeted therapy selection and monitoring of individual's response to treatment.

Increasing evidence in support of phenotype-defining functions of small non-coding RNAs (sncRNAs) prompted a conceptual recognition of informasomes as regulatory RNP complexes of sncRNAs with Argonaute proteins which are mediating information processing, alignment, and integration functions during the flow of genetic information in a cell. Theoretical and experimental considerations support the idea that altered informasome functions may play an important role in the pathogenesis of common human disorders. Individual informasome profiles in a cell are evolving within unique genome-defined context of the sncRNA spectrum, structural/functional features of which are determined by sequence variations. This implies a mechanism of the informasome reprogramming during development and ontogeny which may affect phenotypes in a manner described as a “butterfly” effect in chaotic systems. If this hypothesis is correct, it will open the avenue for development of therapeutic approaches for targeted prevention and/or reversal of SNP “butterfly” effects on phenotypes and informasome reprogramming from pathology-enabling to physiological states. It seems attractive to envision the individual SNP-pattern-related personalized approaches to disease management entailing companion diagnostic tests for individualized therapy selection and disease profile-tailored RNA-based therapeutics for informasome reprogramming. Based on this therapeutic strategies targeting expression of protein-coding “master” disease genes appear particularly promising.

Sequence homology profiling was carried out of the allelic sequences of eight disease-linked SNPs associated with the NLRP1 locus (rs6502867; rs4790797; rs12150220; rs2670660; rs878329; rs7223628; rs8182352; rs4790796) as well as four disease-linked SNPs (rs10181656; rs8179673; rs7574865; rs11889341) which are located within a continuous genomic region associated with the STAT4 gene as shown in Example 1, infra.

The invention will be further described in the following examples, which do not limit the scope of the invention described in the claims

### EXAMPLES

### Example 1

### Materials and Methods

A sequence homology profiling was carried out of 2301 human small non-coding RNAs transcript that were previously identified and are accessible in publicly available databases. 314 intronic transcripts encoded by DNA sequences which are located in regions distal from previously annotated genes (at least 10 kb) as defined in the previously published work were analyzed. The general significance of these findings was validated by analysis of additional set of 629 transintrons identified for the ˜1% of the human genome in the ENCODE regions. A sequence homology profiling was carried out of 71 sncRNA transcripts, including 12 PASRs and 34 TASRs, expression of which was identified by microarray analysis and validated using independent analytical methods such as Northern and/or quantitative RT-PCR. 235 intergenic transcripts were analyzed and identified for the ˜1% of the human genome in the ENCODE regions. DNA sequences encoding these intregenic transcripts are located in regions distal from previously annotated genes (at least 5 kb). Sequence homology profiling was carried out of the 1005 human piRNAs derived from 14 clusters residing on 9 chromosomes. The allelic sequences were analyzed of the 89 master trans-SNP regulatory loci located at 12 distinct chromosomal regions of human genome (11p15; 22q13; 5q31; 5q33; 7q21; 14q32; 20q13; 6p21; 4q11-q35; 4p16; 1p22; 5g13-q14) and affecting expression of the 163 target genes in trans. Utilizing BLASTN algorithm to search for a miRNA in a sequence>100 nt (Wublastn; E value cutoff: 10. For sequences<100 nt, and utilizing SSEARCH algorithm (SSEARCH; E value cutoff: 10, which is useful for finding a short sequence within the library of microRNAs.

The identities of genes representing potential targets for corresponding microRNAs were obtained using the TargetScan database. The sequences of the stem-loop and mature microRNAs were retrieved from the Hairpin and Mature databases, respectively, of the MirBase. The identities of all sequences were validated using the BLASTN program to search nucleotide databases using a nucleotide query. All analyzed sequences and computational tools reported in this study are publicly available as web-accessible resources.

Additionally, sequence homology profiling of the allelic sequences of the 81 SNP loci located at distinct chromosomal regions of human genome and manifesting most significant associations with seven common human diseases was performed.

Further, sequence homology profiling of the allelic sequences of the 93 SNP loci located at distinct chromosomal regions of human genome and manifesting most significant associations with seven common human diseases was also carried out. The sequence homology was performed profiling of independent sets of 23 SNPs with most significant evidence for associations with type 2 diabetes as well as an independent set of 16 disease-linked SNPs identified in recent high-powered GWA studies of RA patients which unequivocally confirmed five RA susceptibility genes namely HLA-DRB1, PTPN22, OLIG3/TNFAIP3, STAT4 and the TRAF1/C5. In addition, sequence homology profiling of 18 AITD-linked SNPs; 15 MS-linked SNPs; 12 SNPs associated with autoimmune disorders (AID); 20 AS-linked SNPs; 16 BC-linked SNPs; 18-SLE-linked SNPs; 18 PC-linked SNPs; 18 vitiligo-associated multiple autoimmune disease SNPs (VIT) and 5 UC-linked SNPs which were identified recently in multiple high-power association studies was also carried out.

Gene expression analysis data of peripheral blood mononuclear cells (PBMCs) from Crohn's disease (CD), rheumatoid arthritis (RA), spondyloarthropathy (SA), and ulcerative colitis (UC) patients; synovial fluid mononuclear cells of patients with RA and SA; kidneys of patients with type 2 diabetic nephropathy (T2D); as well as of dorsolateral prefrontal cortex from patients with bipolar (BP) disorder were obtained from the GEO database (accession numbers GDS1615, GDS961, GDS711, and GDS2190). The expression data for two most significantly differentially regulated probe sets representing the KPNA1 gene mRNA is shown (202059_s_at for CD and UC; 40474_r_at for T2D, SA, and RA; and 202058_s_at for BD). All analyzed sequences and computational tools reported in this study are publicly available as web-accessible resources.

The Affymetrix data sets of the control subjects, experimentally infected individuals, and malaria patients before and after chloroquine therapy were previously reported and can be accessed under accession numbers GSE5418.

Sequence homology profiling of the allelic sequences of eight disease-linked SNPs associated with the NLRP1 locus (rs6502867; rs4790797; rs12150220; rs2670660; rs878329; rs7223628; rs8182352; rs4790796) as well as four disease-linked SNPs (rs10181656; rs8179673; rs7574865; rs11889341) which are located within a continuous genomic region associated with the STAT4 gene was also carried out.

The mRNA-targeting potential of individual microRNAs was estimated against specific mRNA targets using the values of the context scores as defined by the TargetScan algorithm according to which the lower values of the context scores reflect the higher mRNA targeting potency of a microRNA. To calculate formal numerical values reflecting the mRNA-targeting potency of a given microRNA within the allele-specific context of a homologous disease-linked SNP, the microRNA/mRNA pair-specific context score was multiplied by the allele-specific microRNA/SNP sequence homology e-value, so the relationships between the lower values of the calculated allele-specific microRNA/mRNA context scores and higher mRNA targeting potency of a given microRNA would be maintained. Cumulative mRNA-targeting scores for disease states were obtained by adding individual mRNA-targeting scores calculated for each microRNAs within the context of high-risk SNP alleles. Cumulative mRNA-targeting scores for control alleles were obtained by adding individual mRNA-targeting scores calculated for each microRNAs within the context of low-risk SNP alleles. The significance of associations between the allele-specific microRNA/mRNA targeting scores and mRNA expression values in control and disease states was estimated using the Pearson correlation coefficients. Analyses of both raw microarray expression data and mRNA expression values normalized to controls were carried out and the most significant p values are reported.

The expression data for most significantly differentially regulated probe sets representing corresponding mRNAs are shown. Gene expression signature analysis was performed using previously reported Pearson correlation method. Briefly, each gene expression signature was designed as multidimensional reference vector (MRV) numerical values of which are represented by the log 10-transformed ratios of the average expression values for individual genes in a disease cohort versus control group. Signature score values for individual patients were calculated as a Pearson correlation coefficient of the MRV versus corresponding normalized log 10-transformed gene expression measurements of each patient. Genes comprising the ten-gene CD signature are: ACAN; WNT5A; MMP14; HOXA11; EN1; DICER1; TSC1; MYB; MYBL1; HMGA1; genes comprising the ten-gene RA signature are: ACAN; WNT5A; MMP14; HOXA11; CEBPB; DICER1; TSC1; MYB; MYBL1; PTEN.

### Example 2

### Practical Utility of Application of the Disease Phenocode Concept to Individual Human Disorders

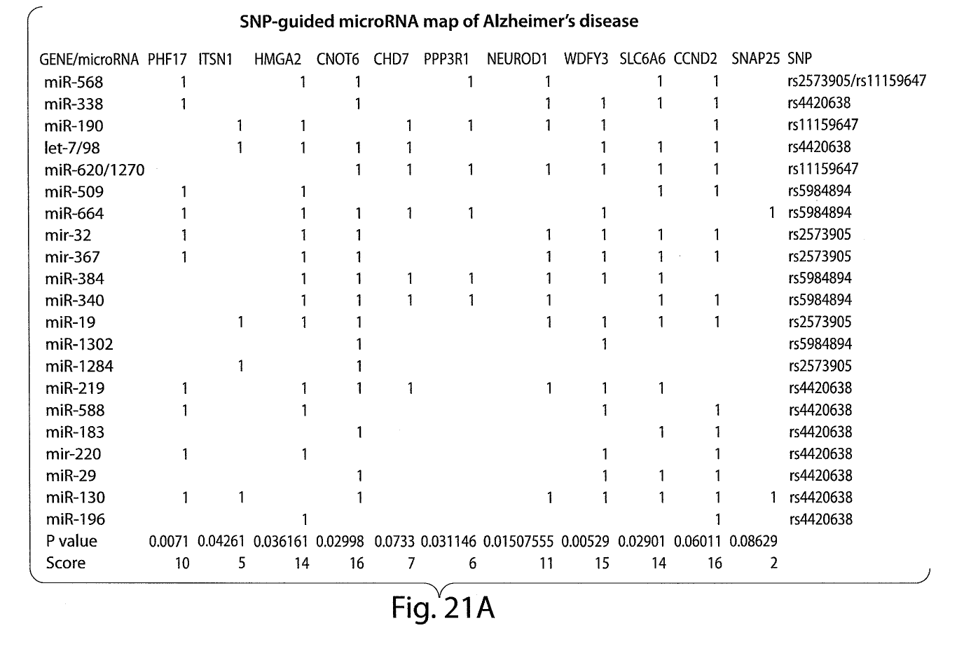

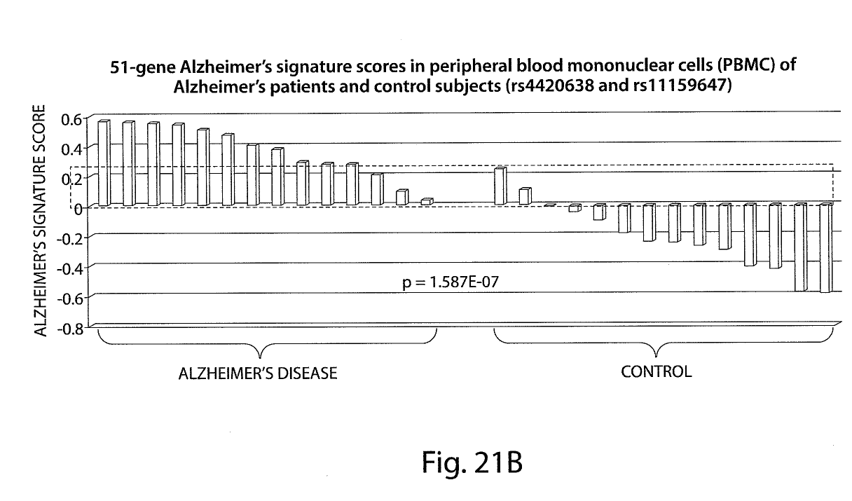

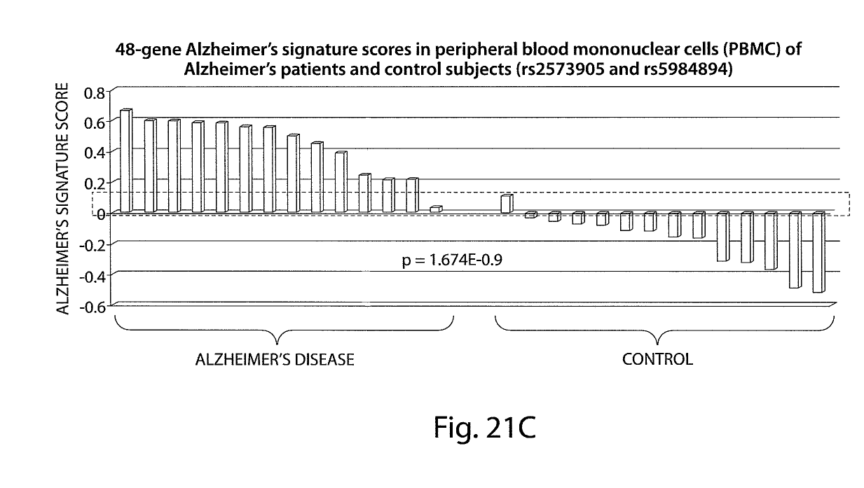

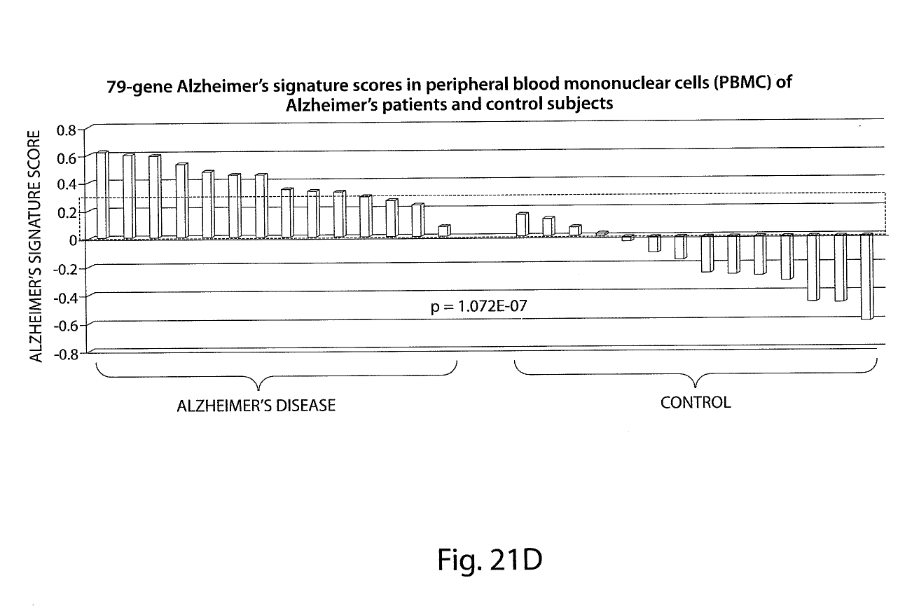

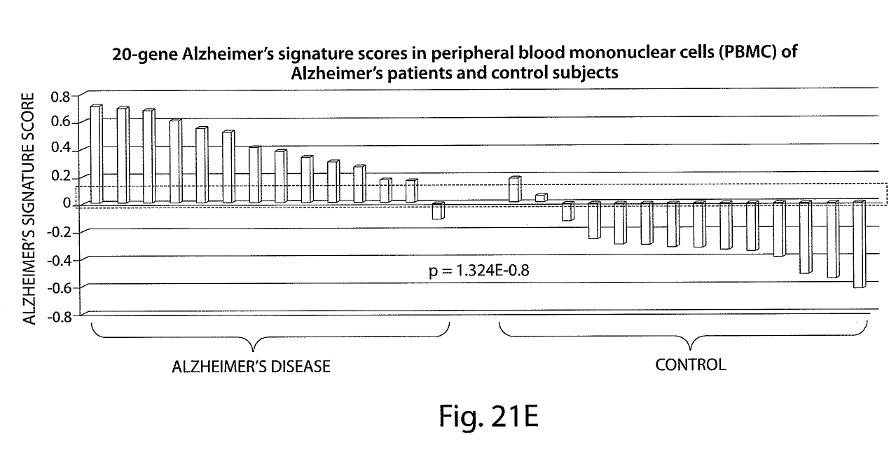

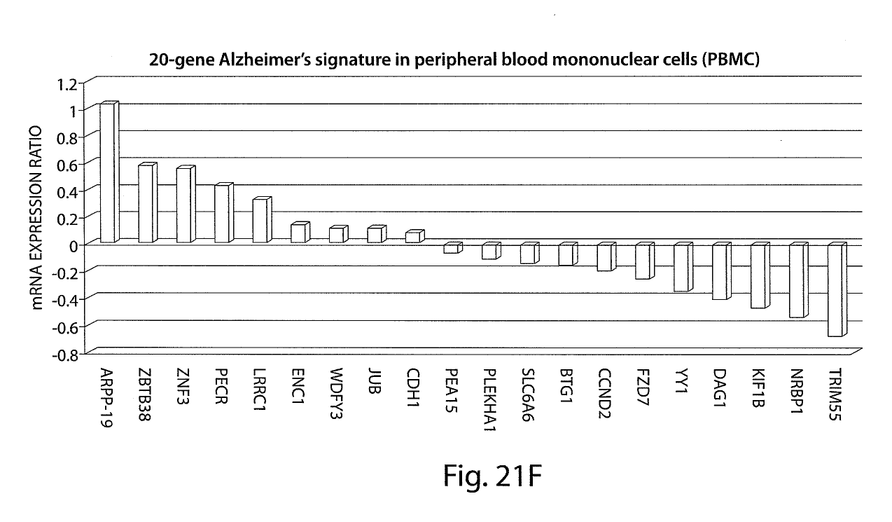

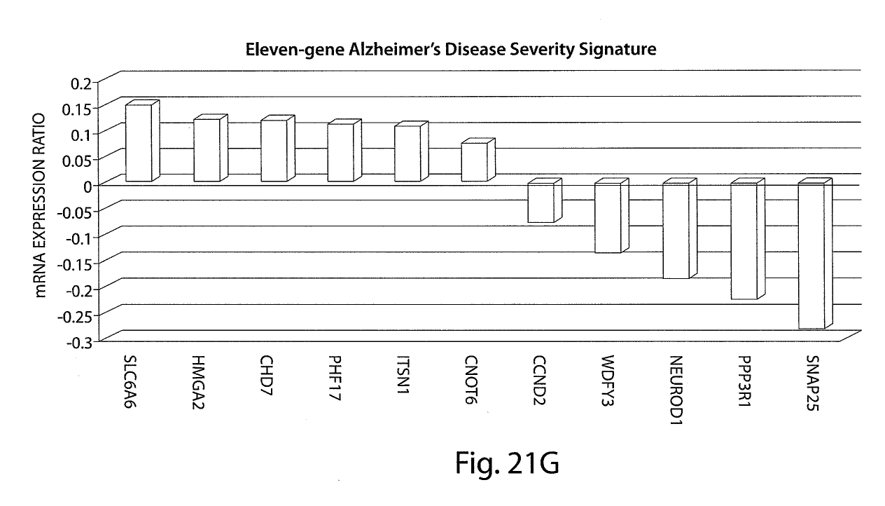

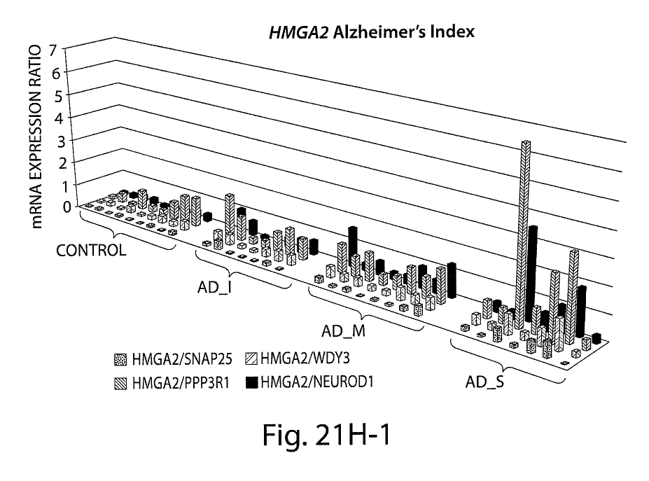

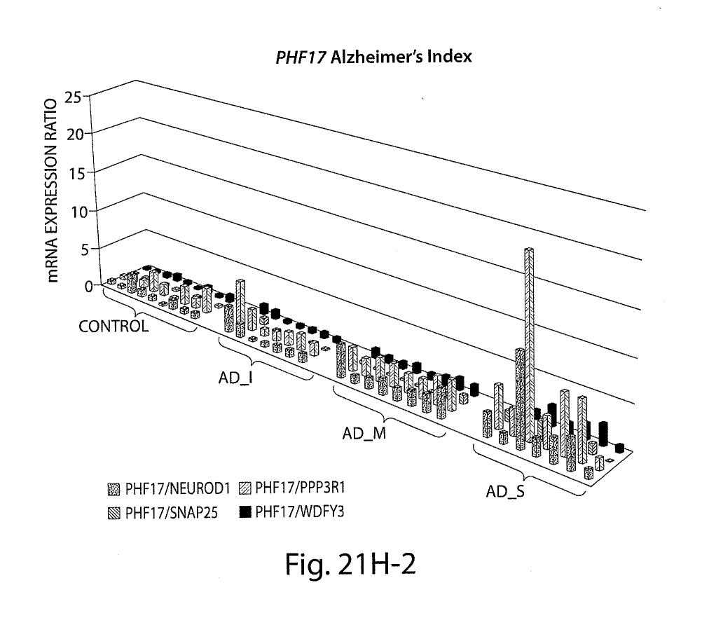

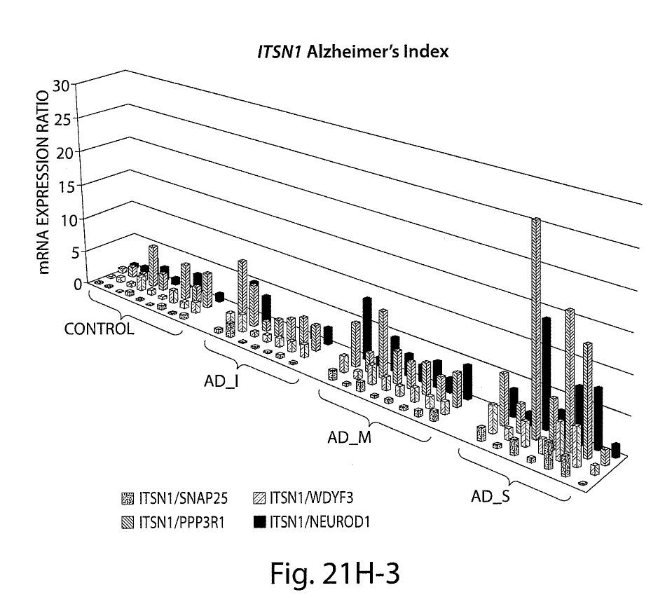

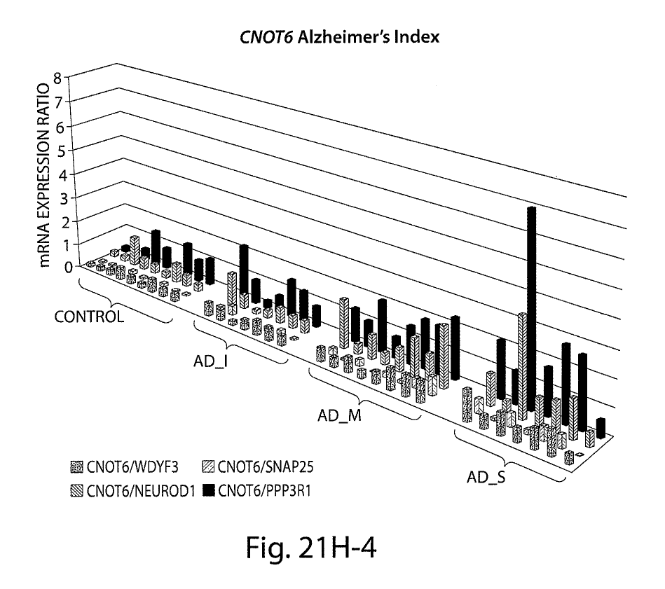

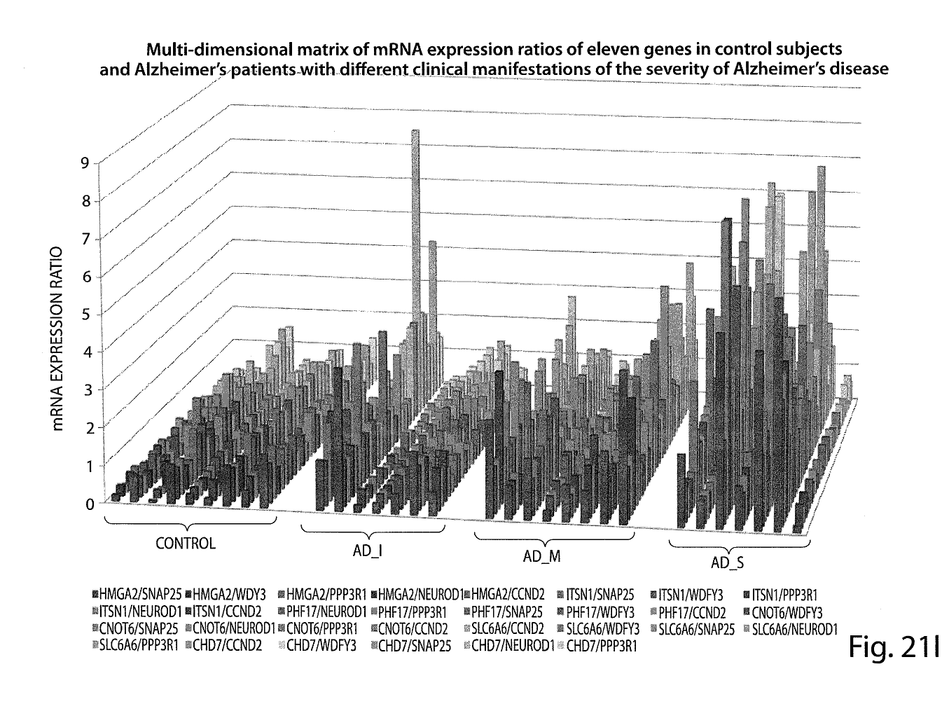

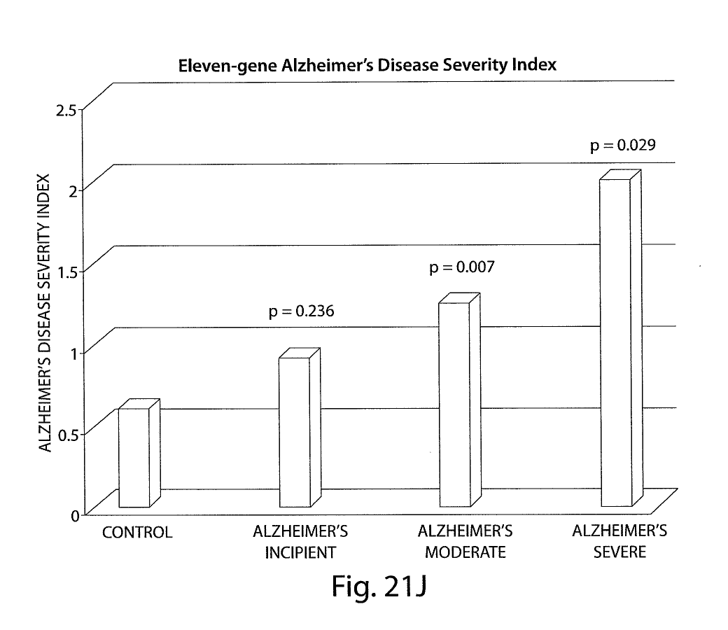

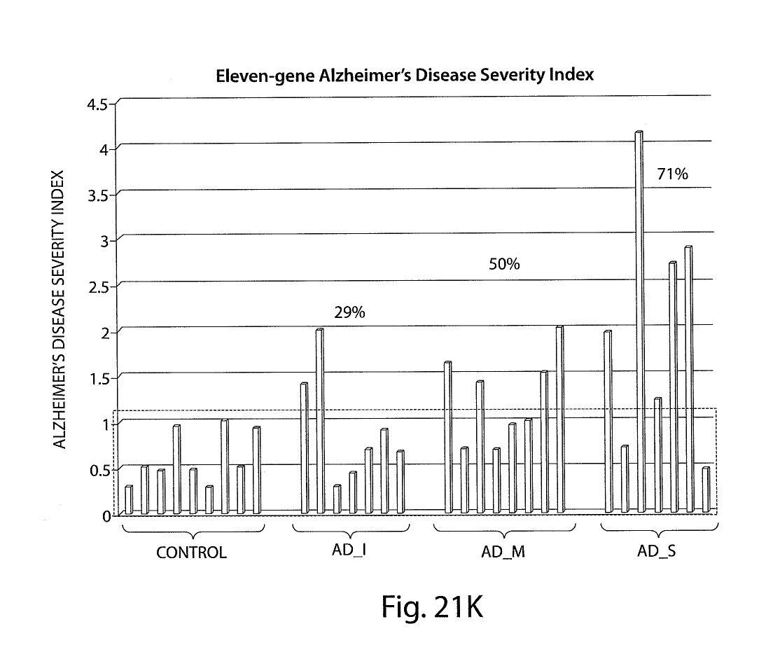

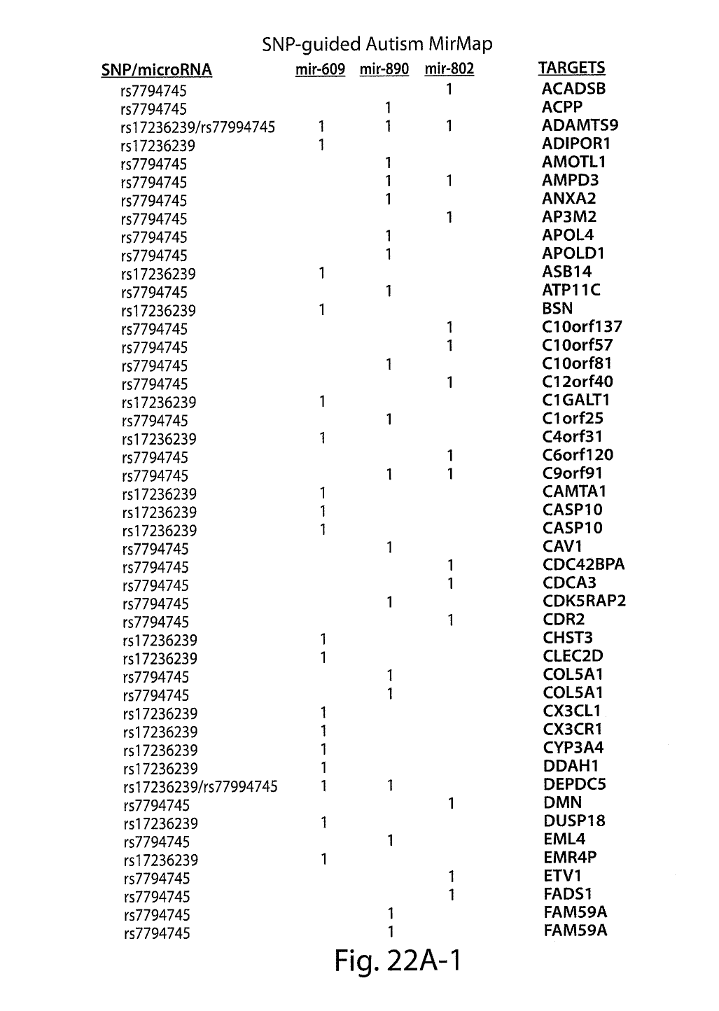

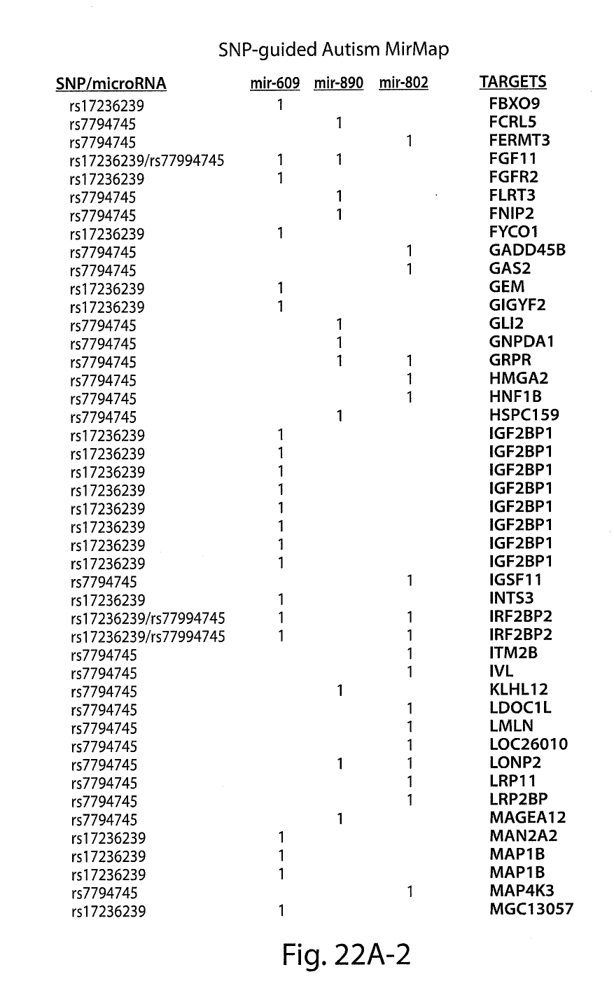

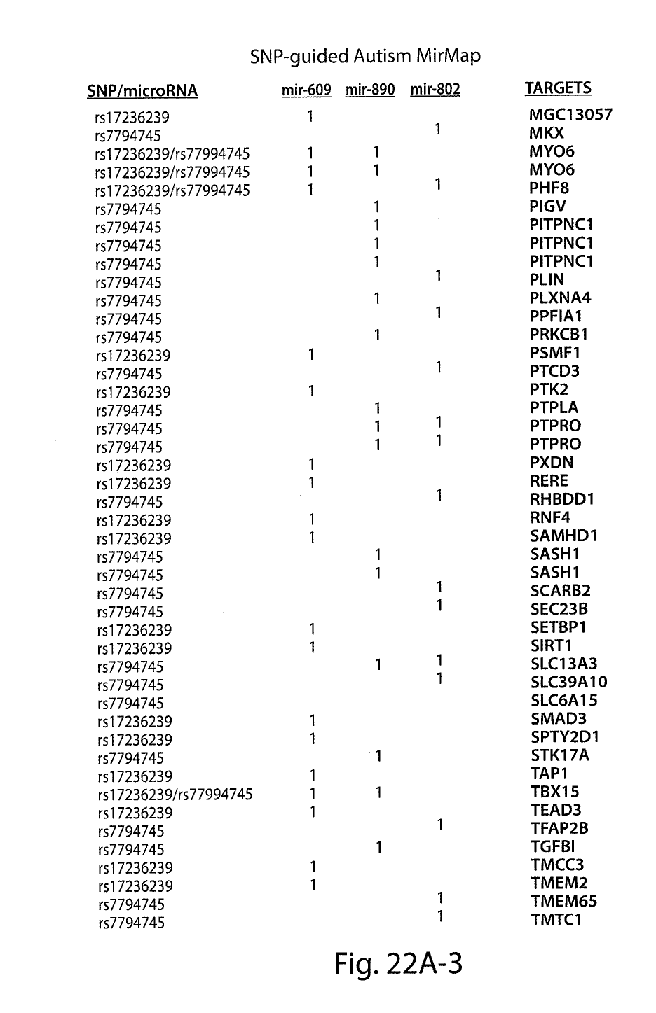

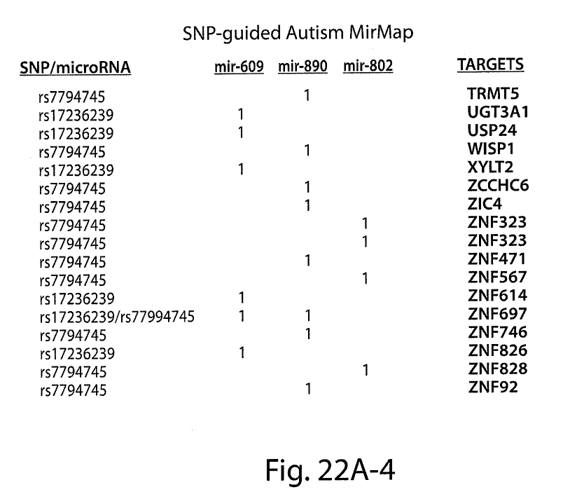

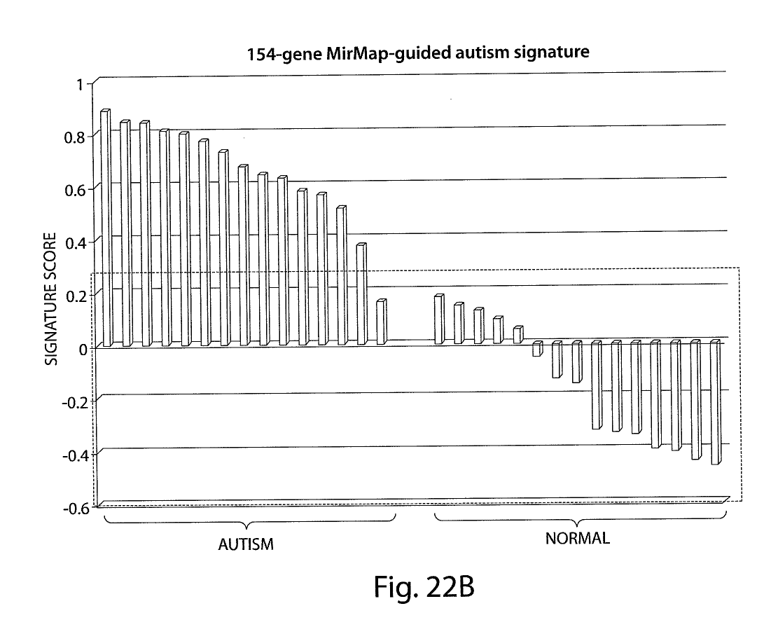

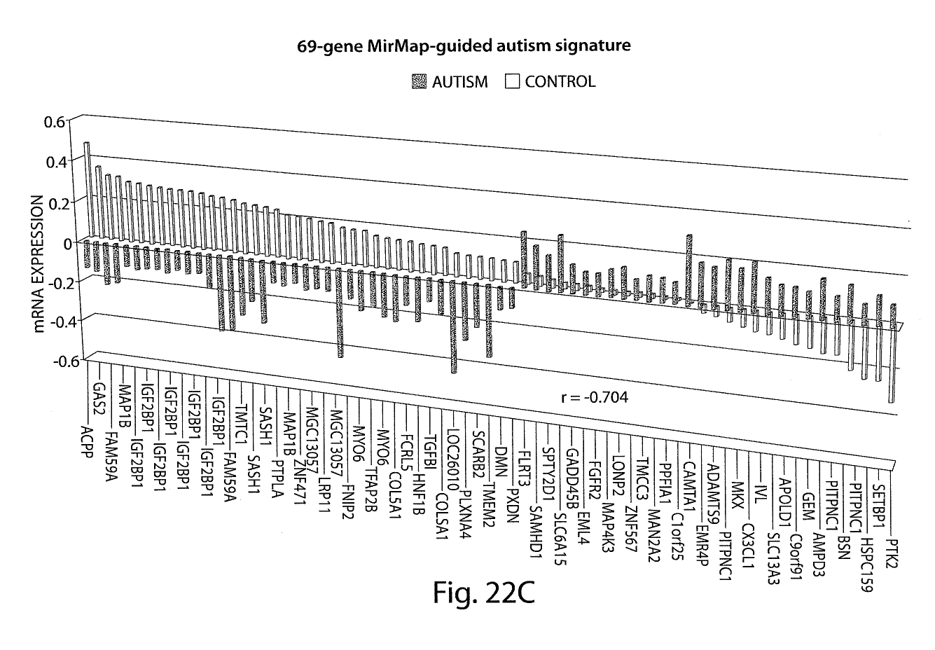

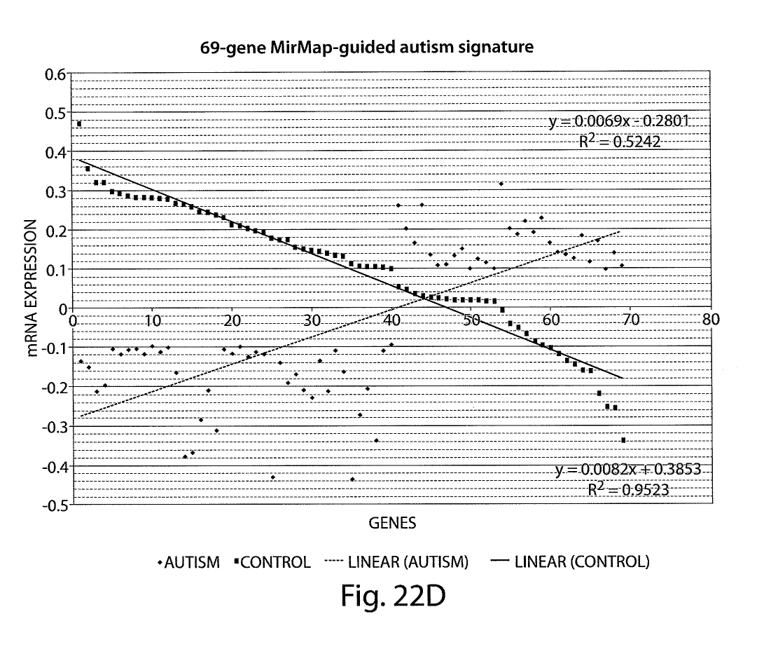

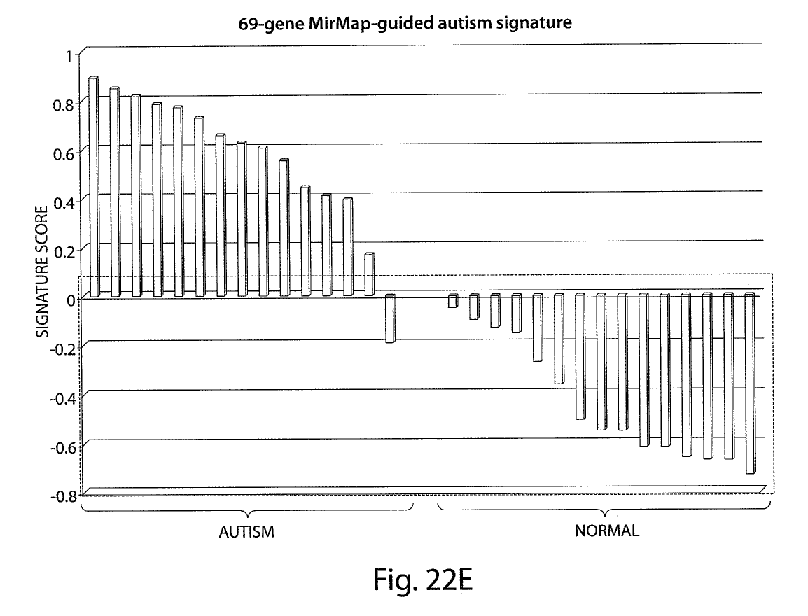

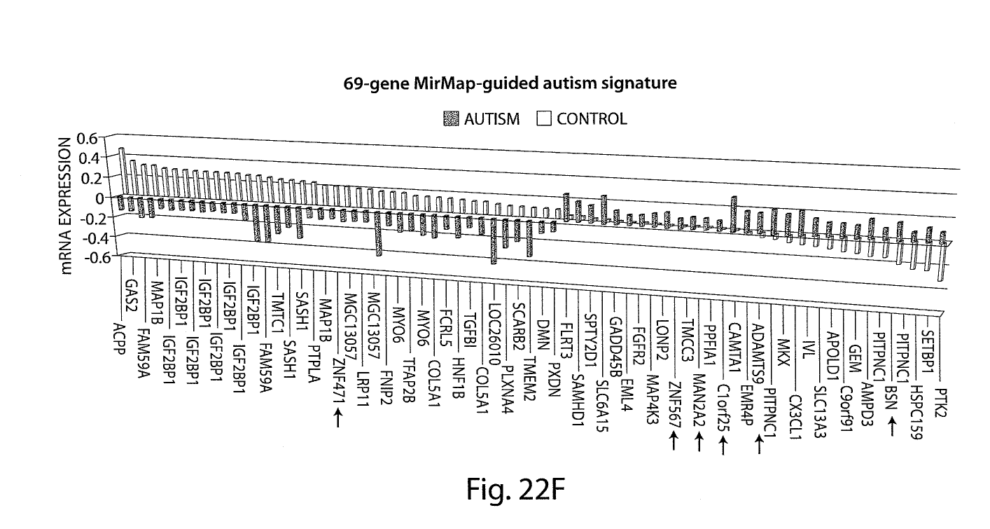

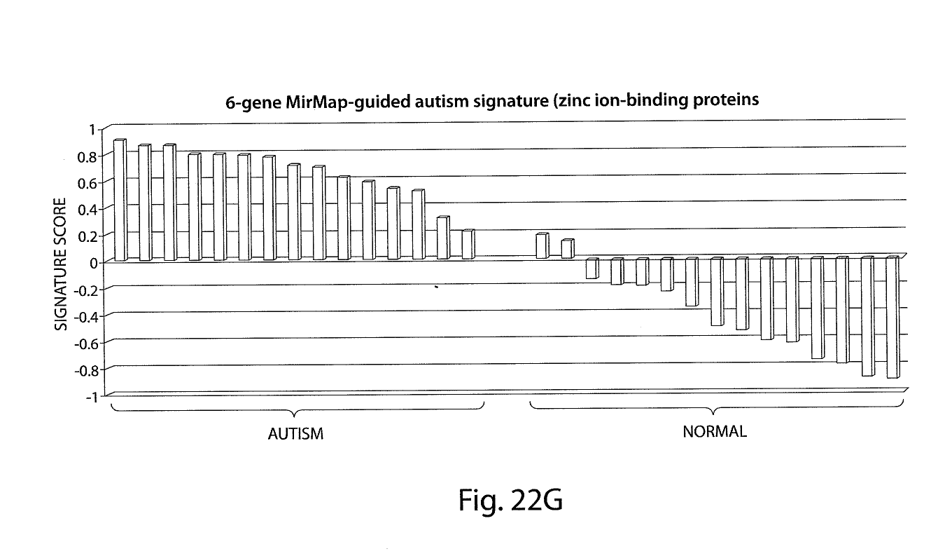

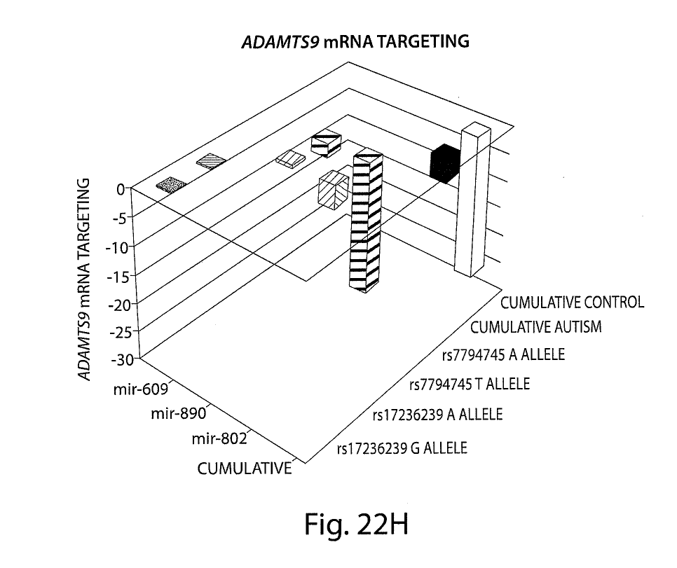

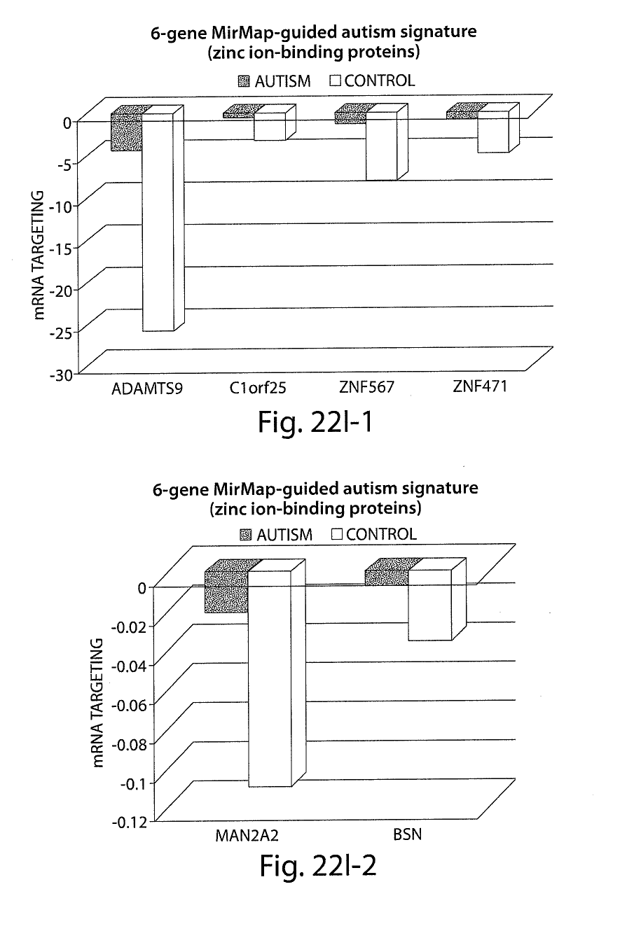

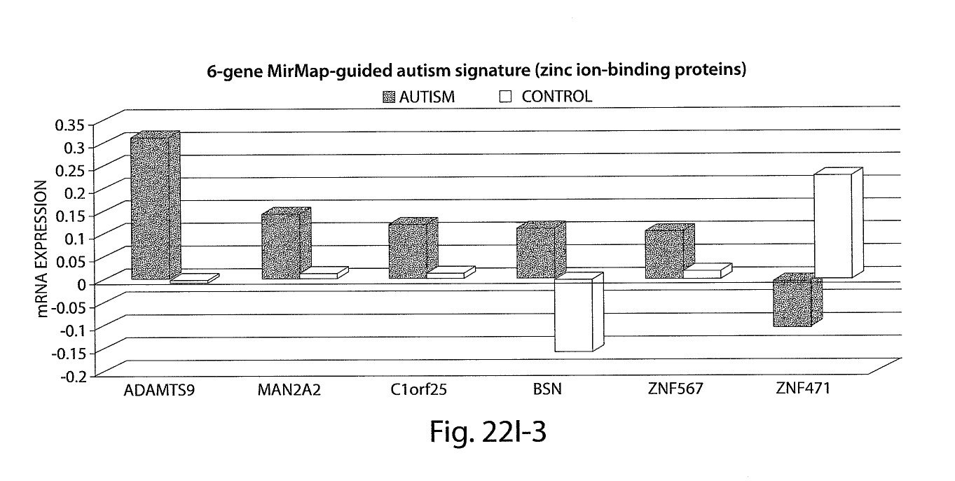

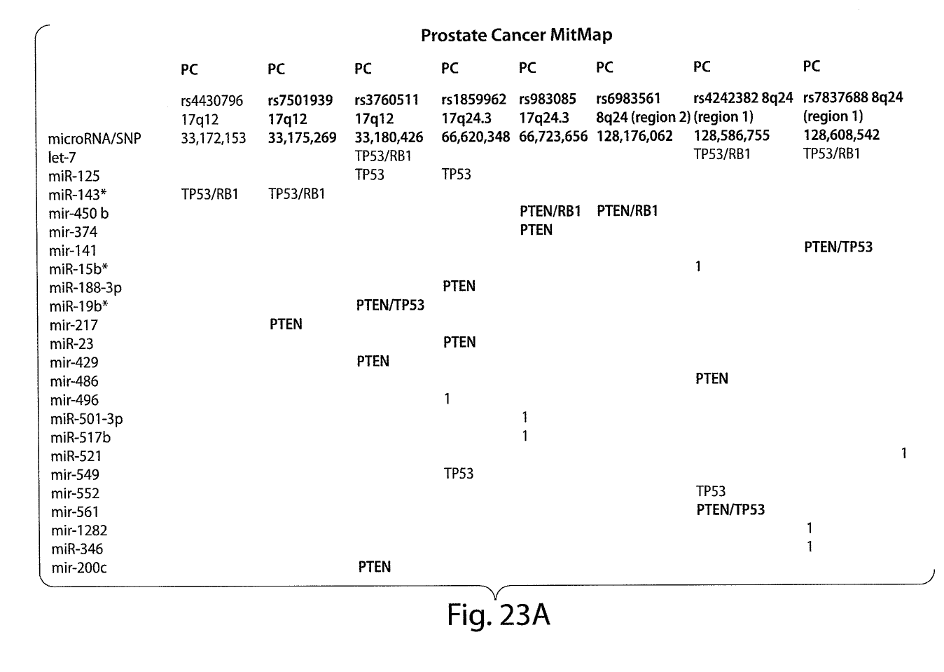

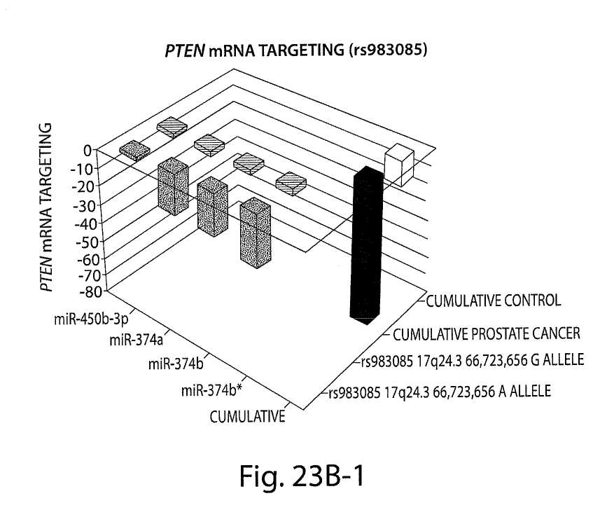

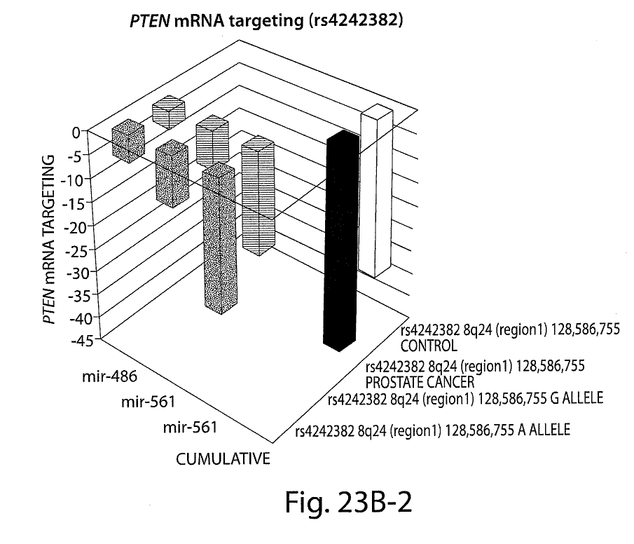

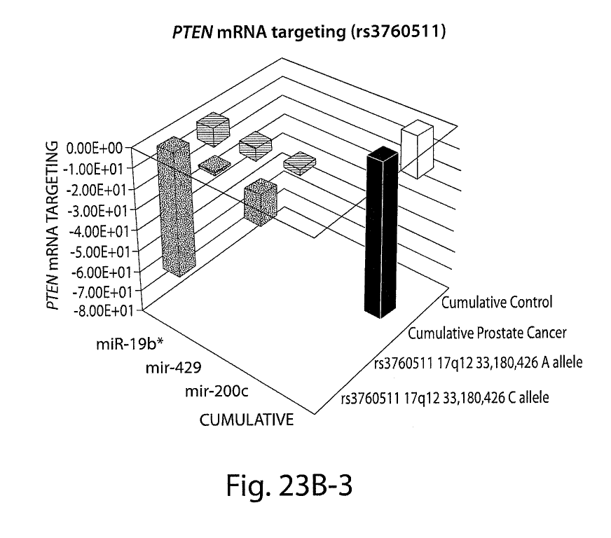

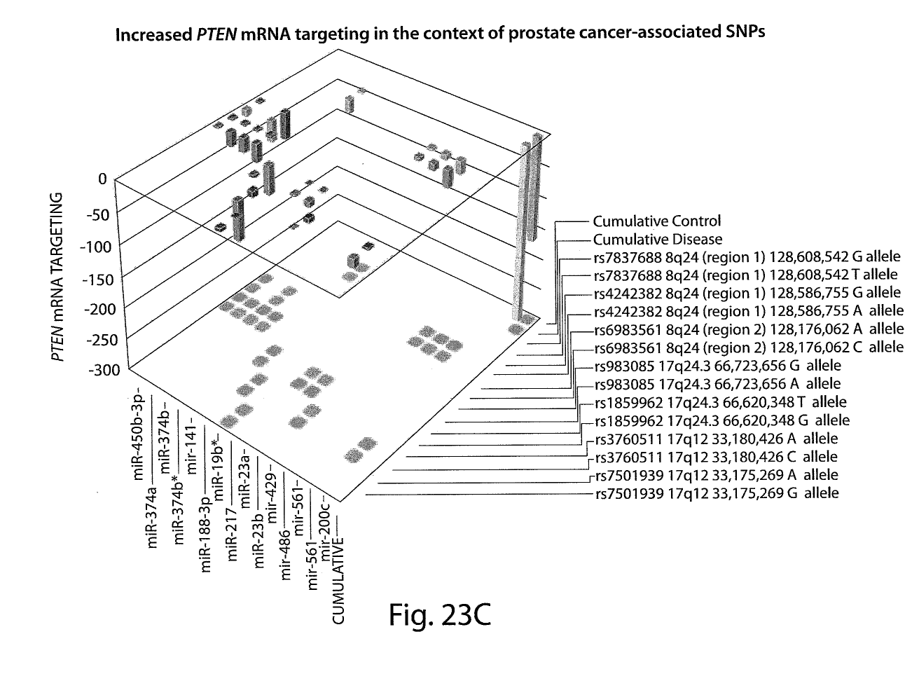

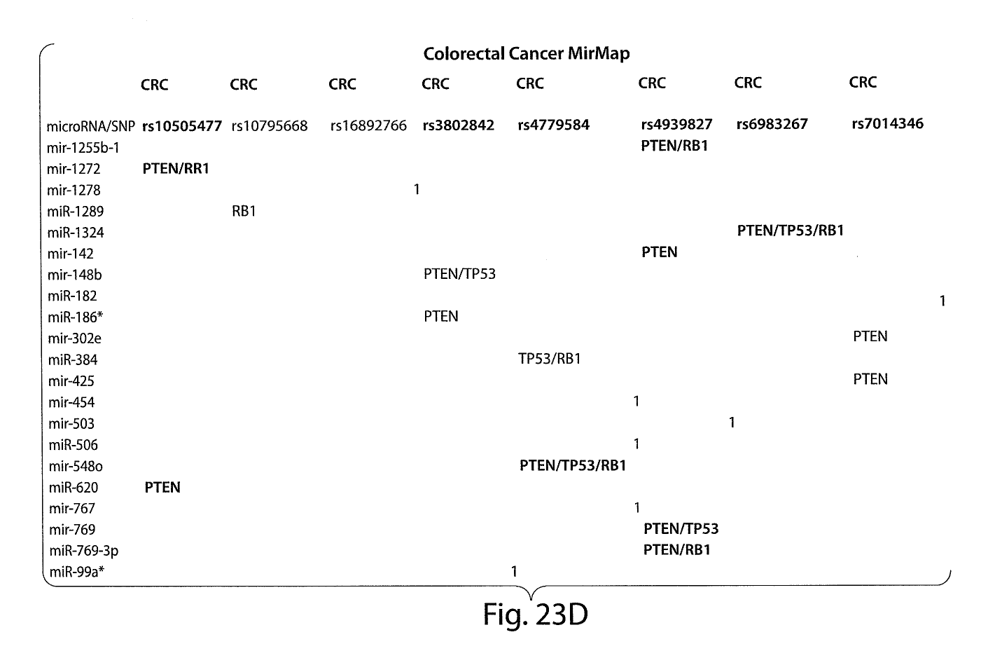

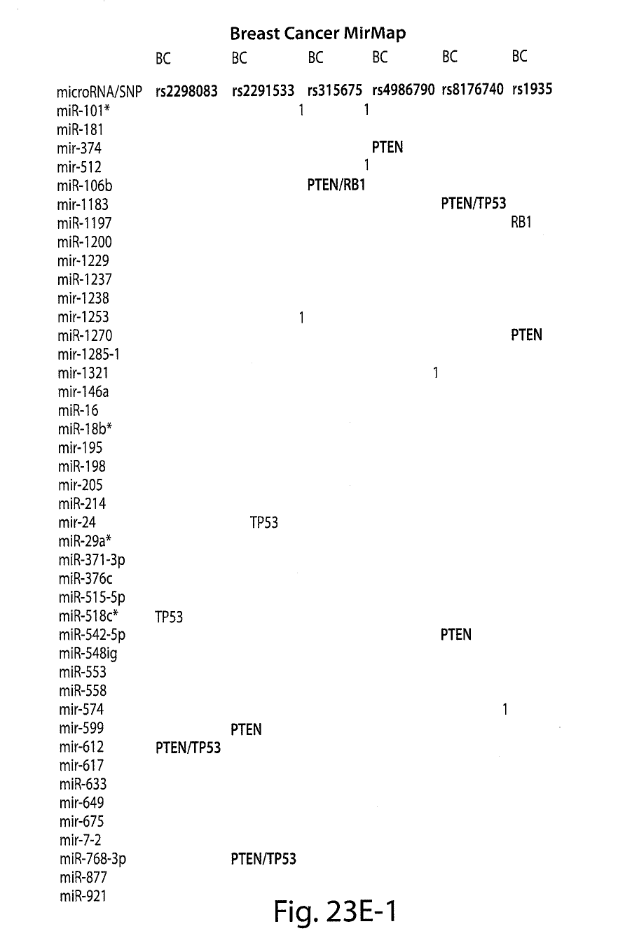

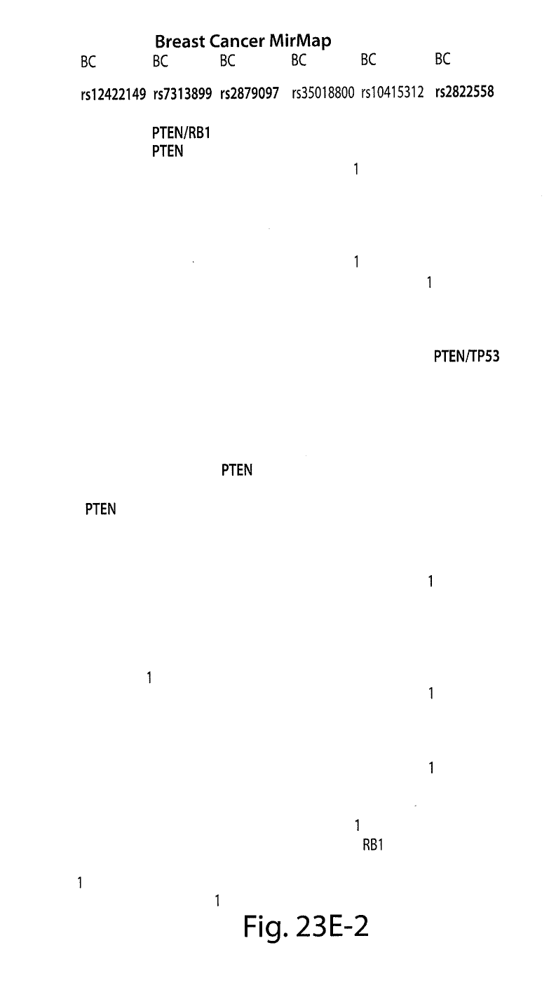

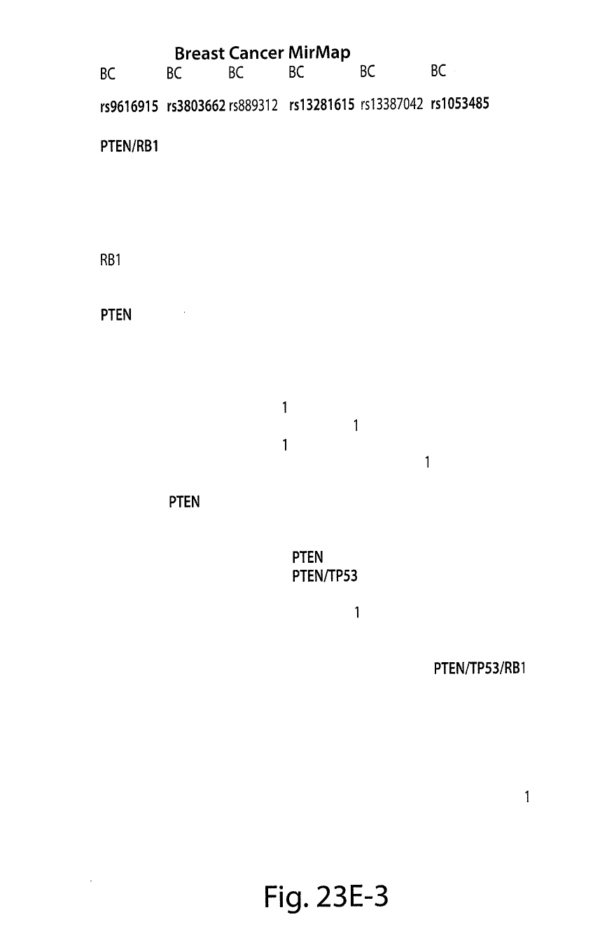

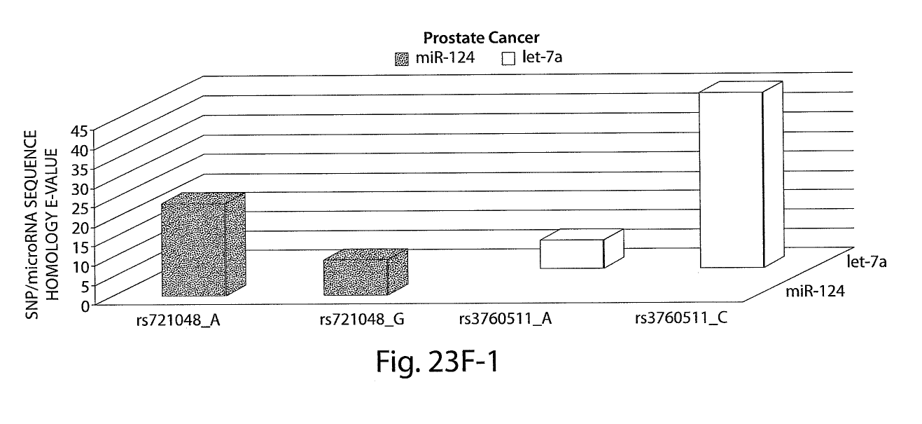

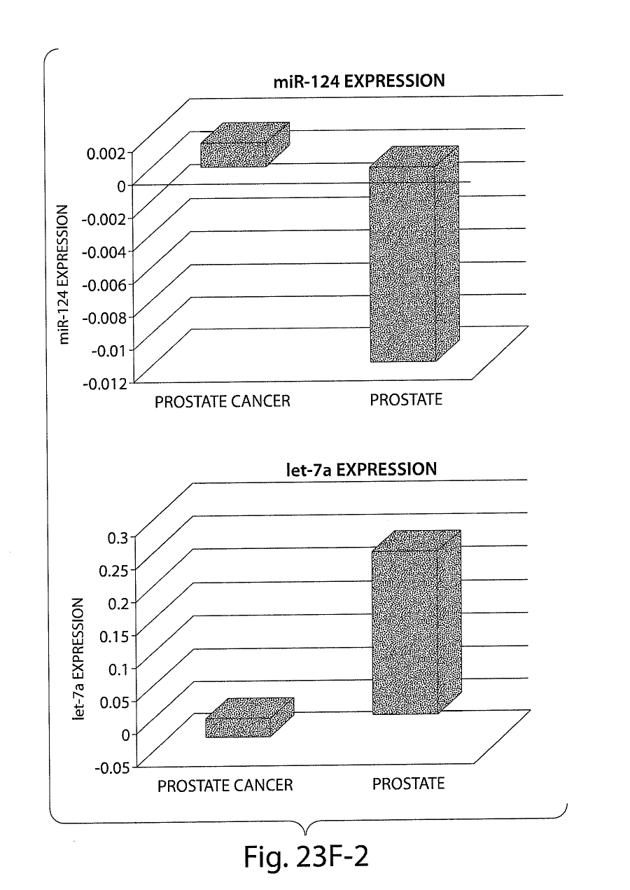

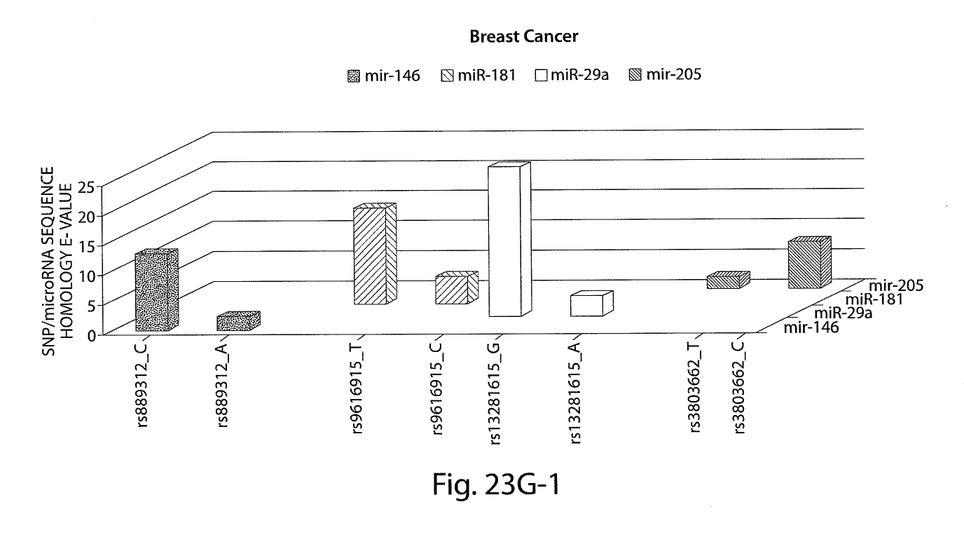

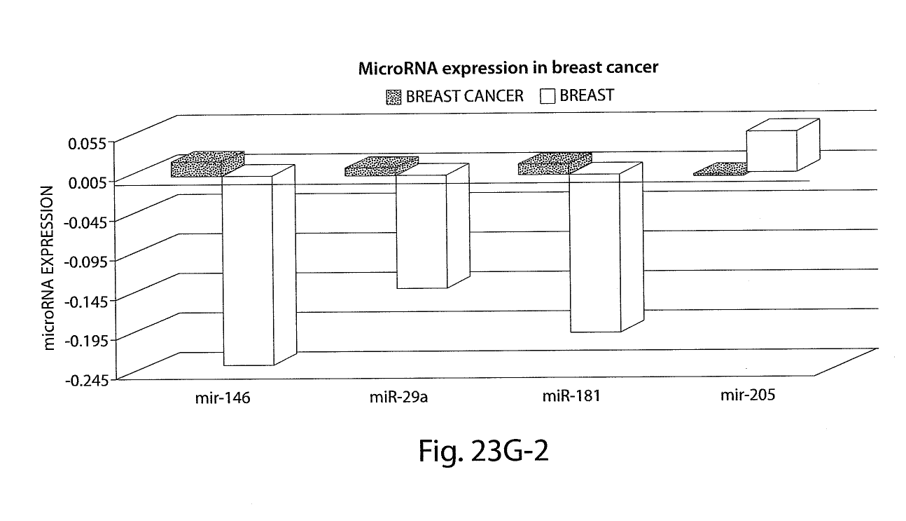

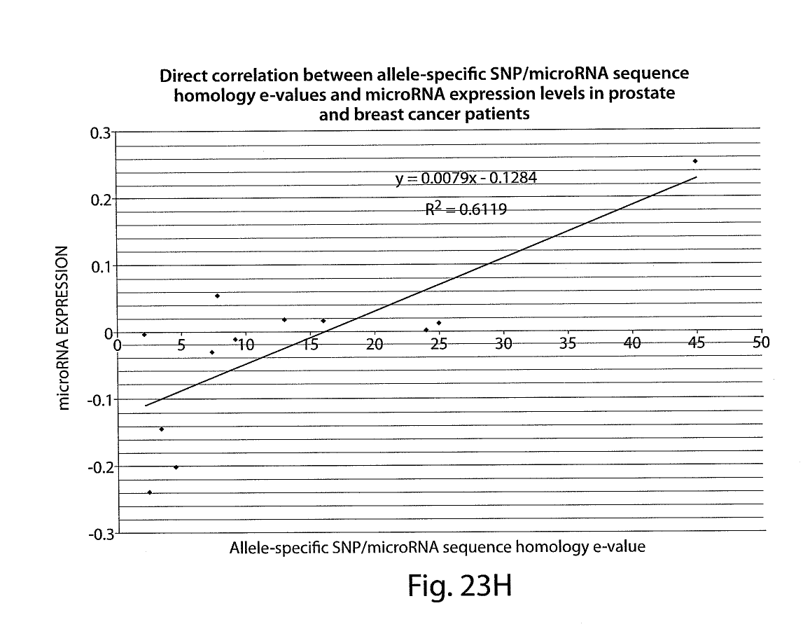

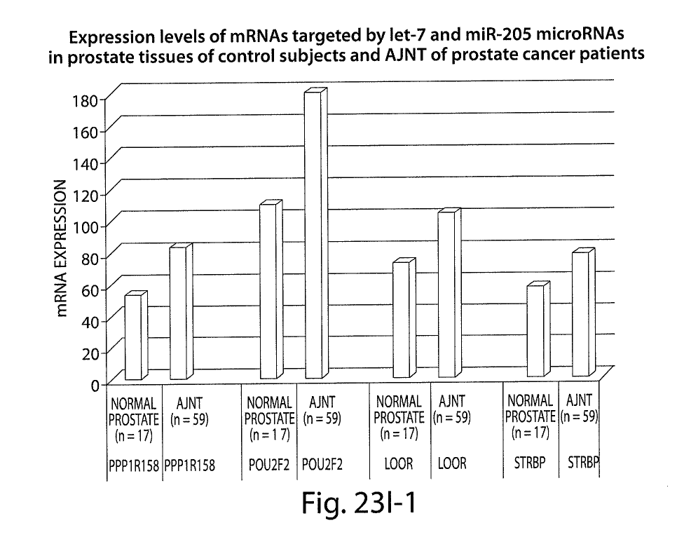

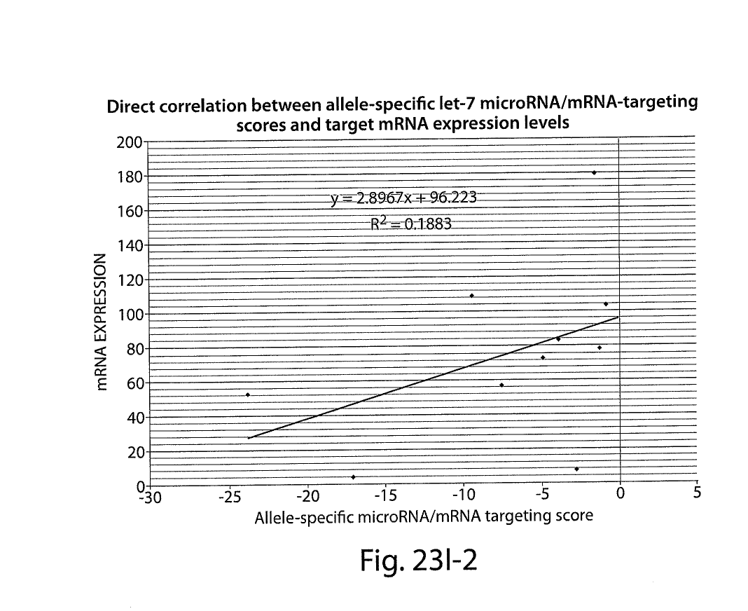

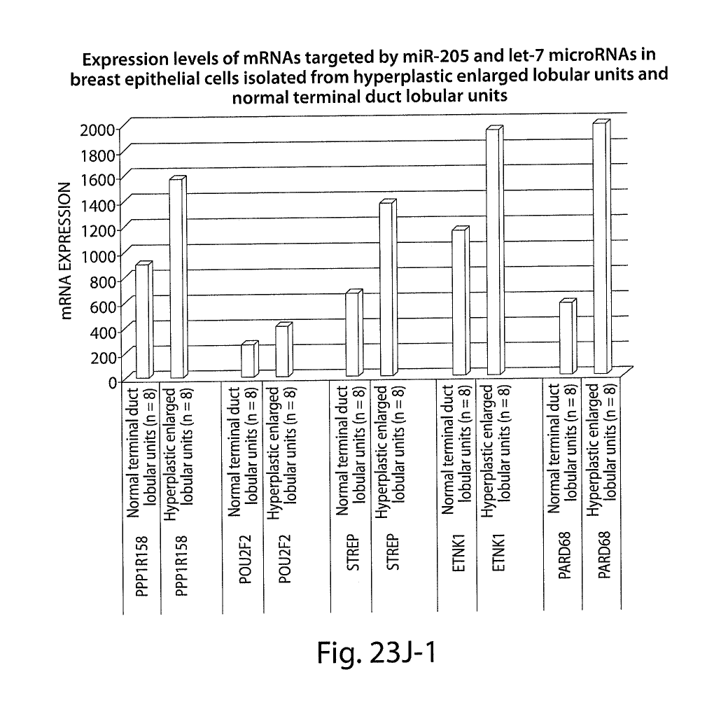

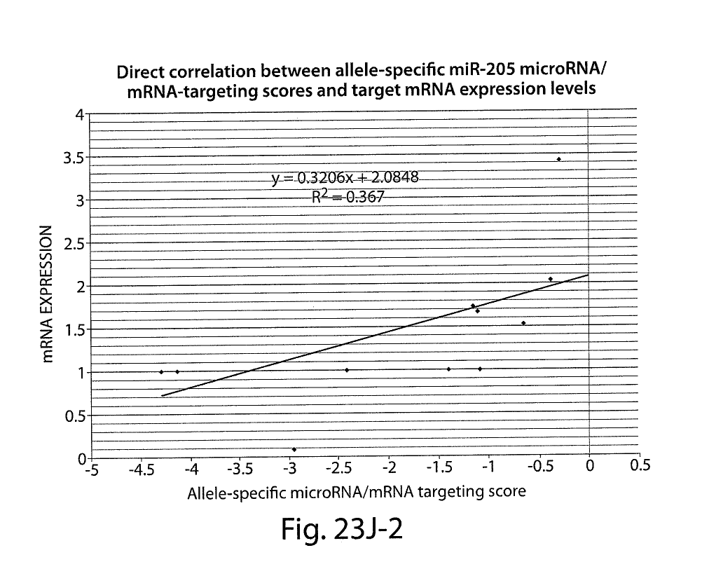

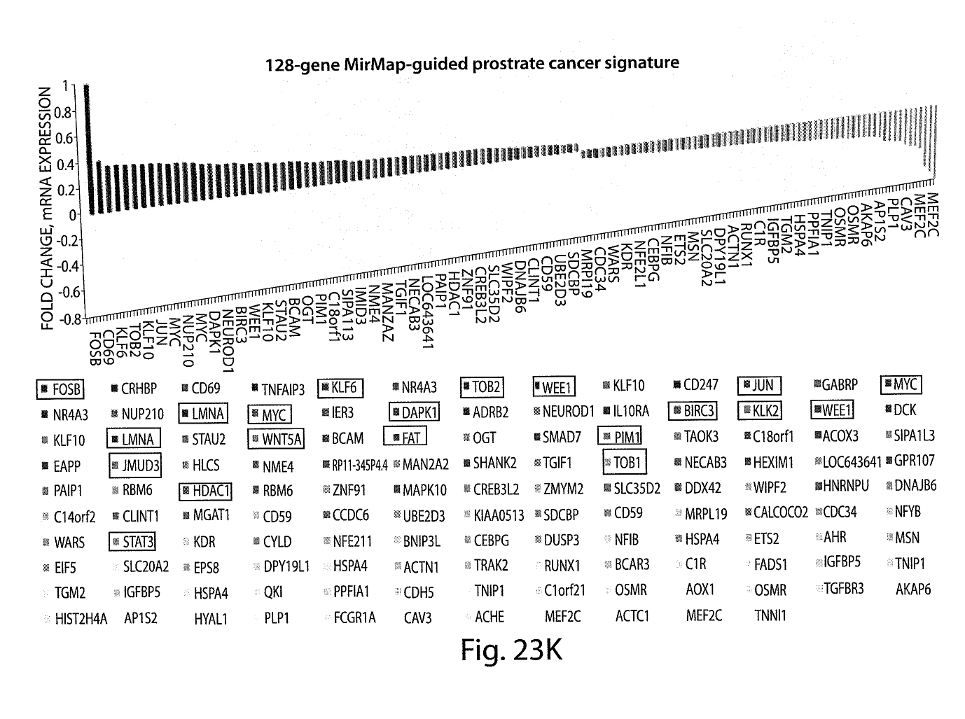

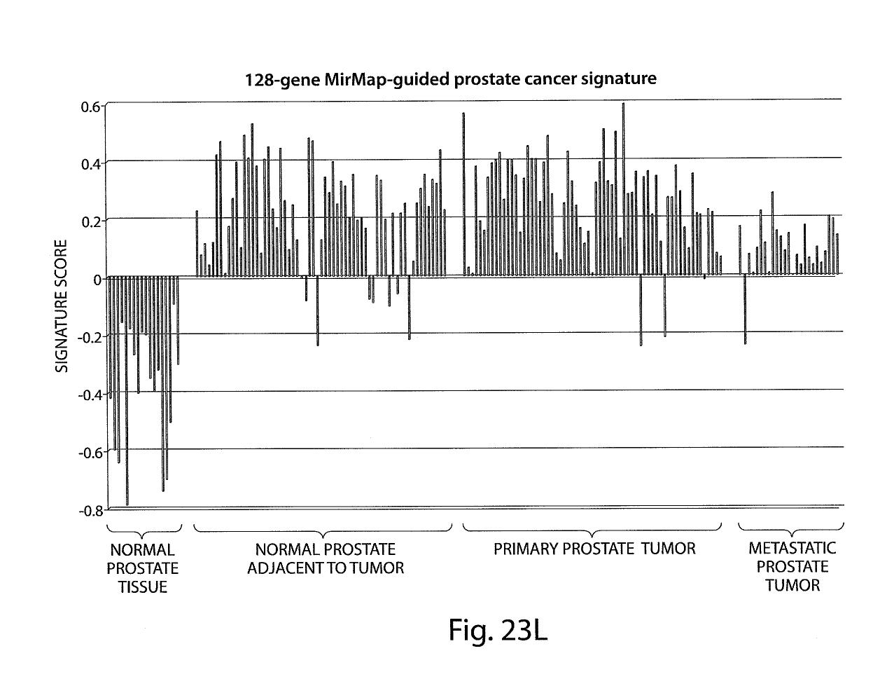

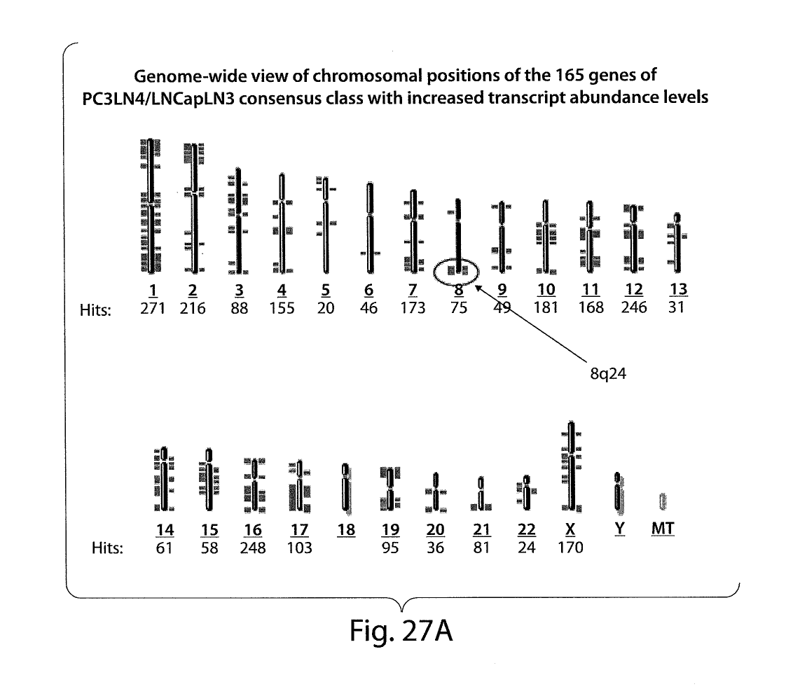

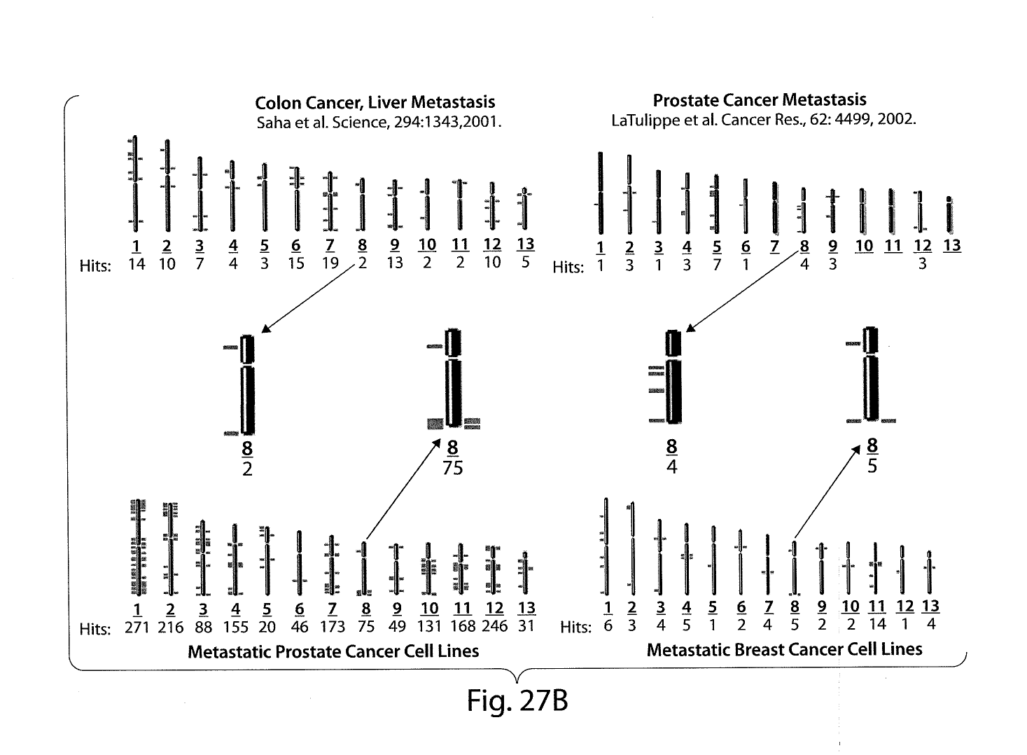

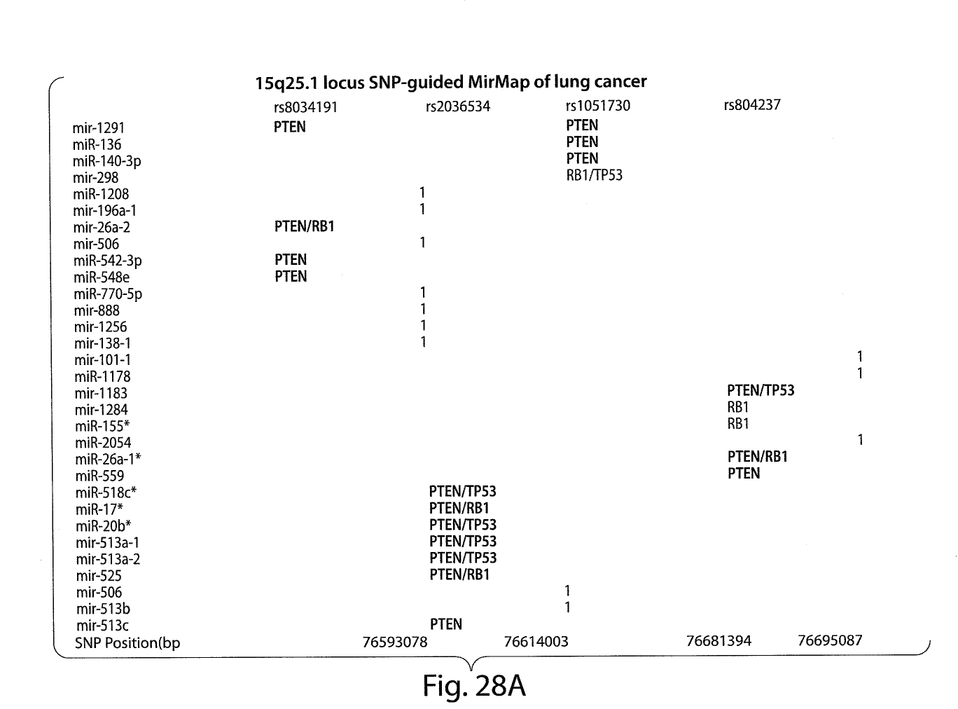

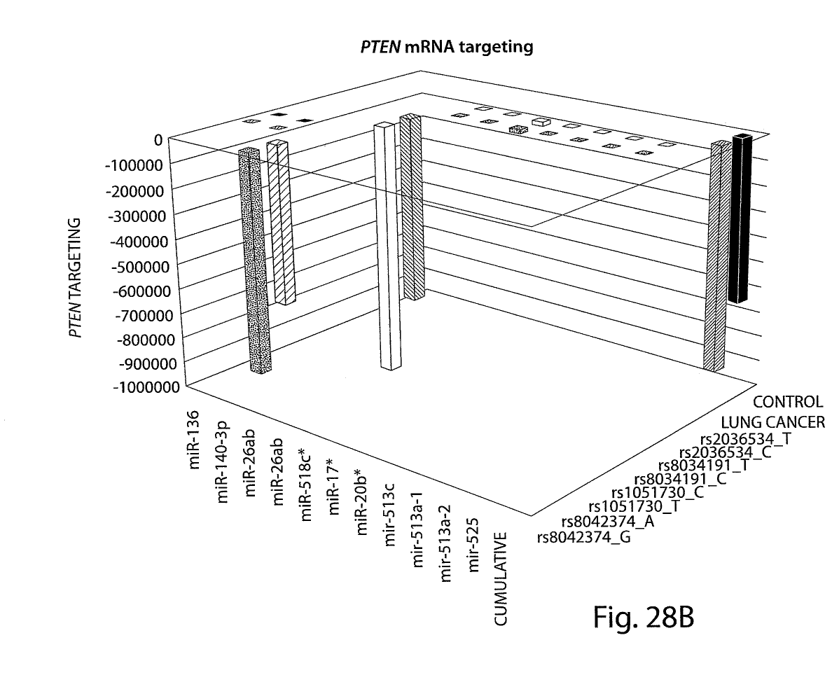

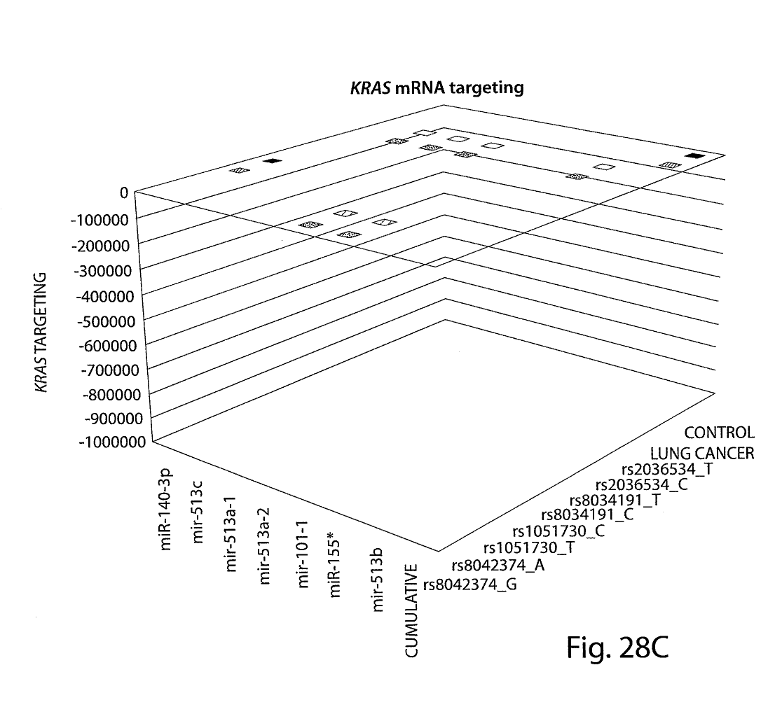

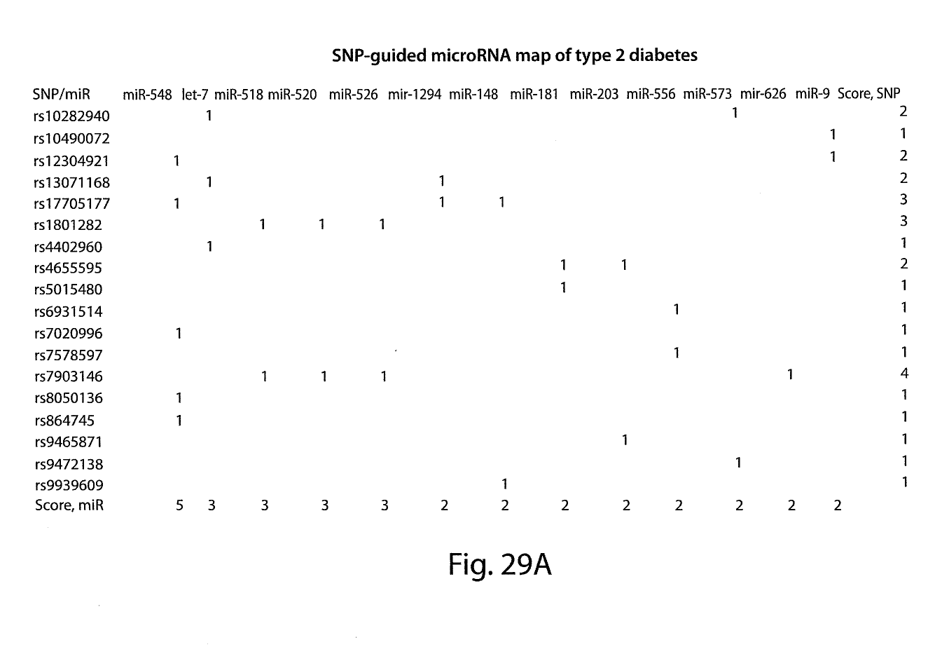

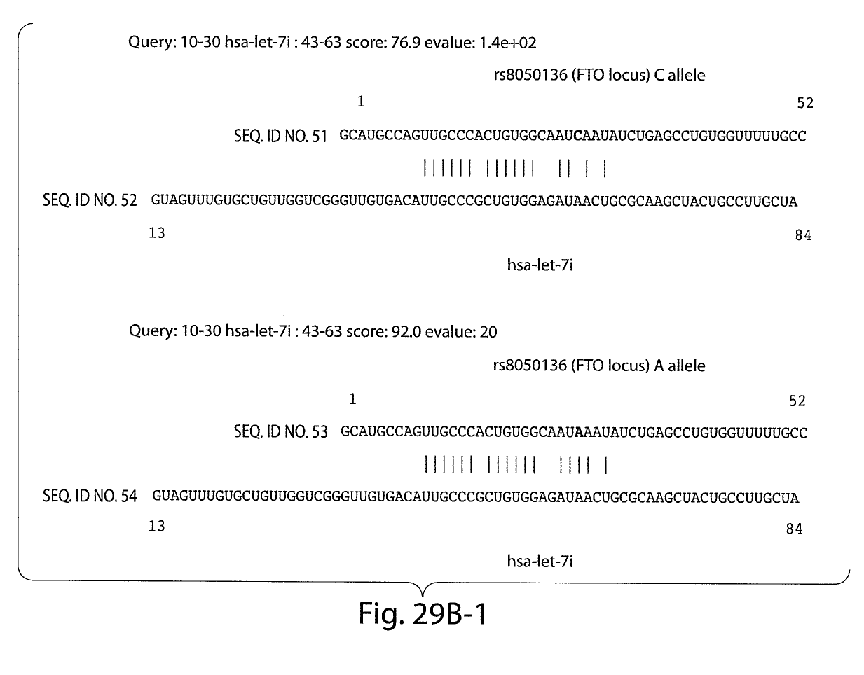

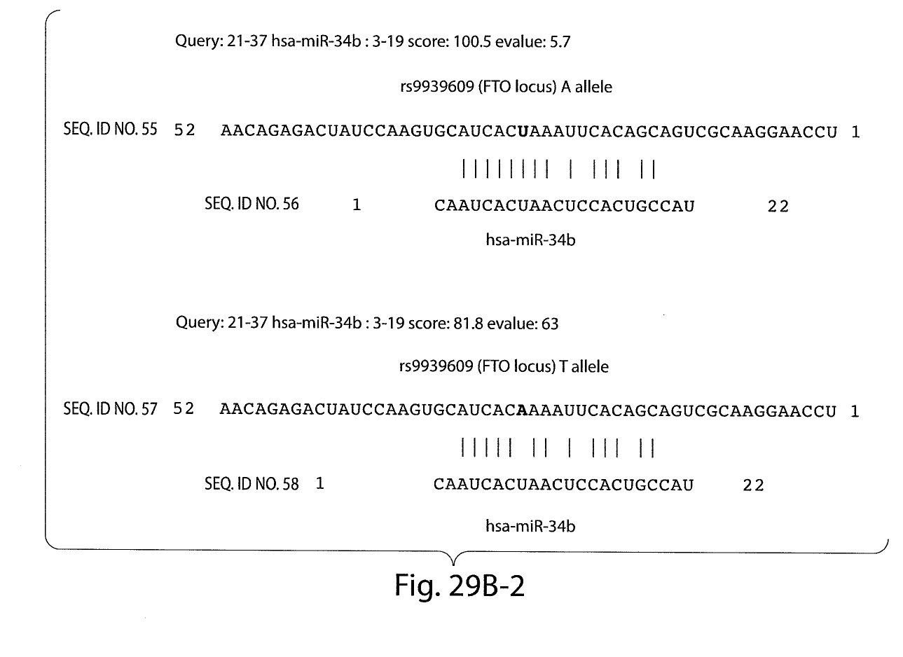

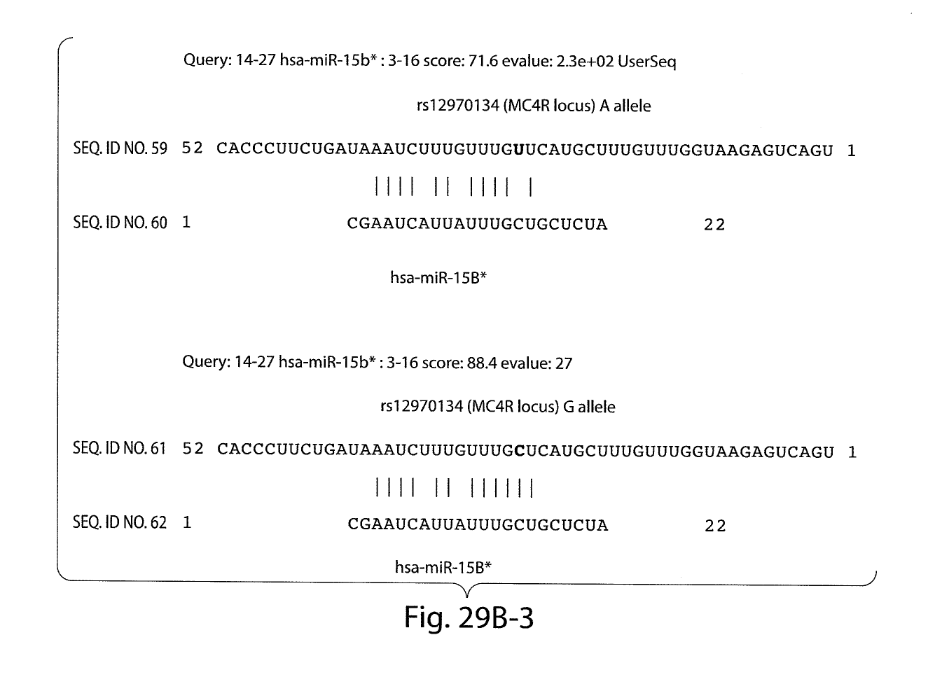

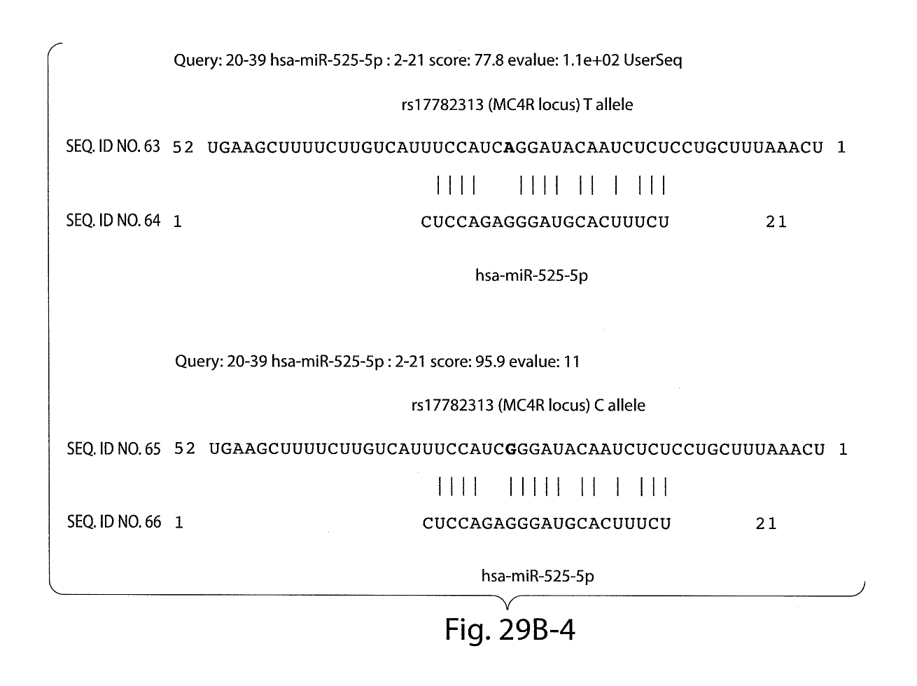

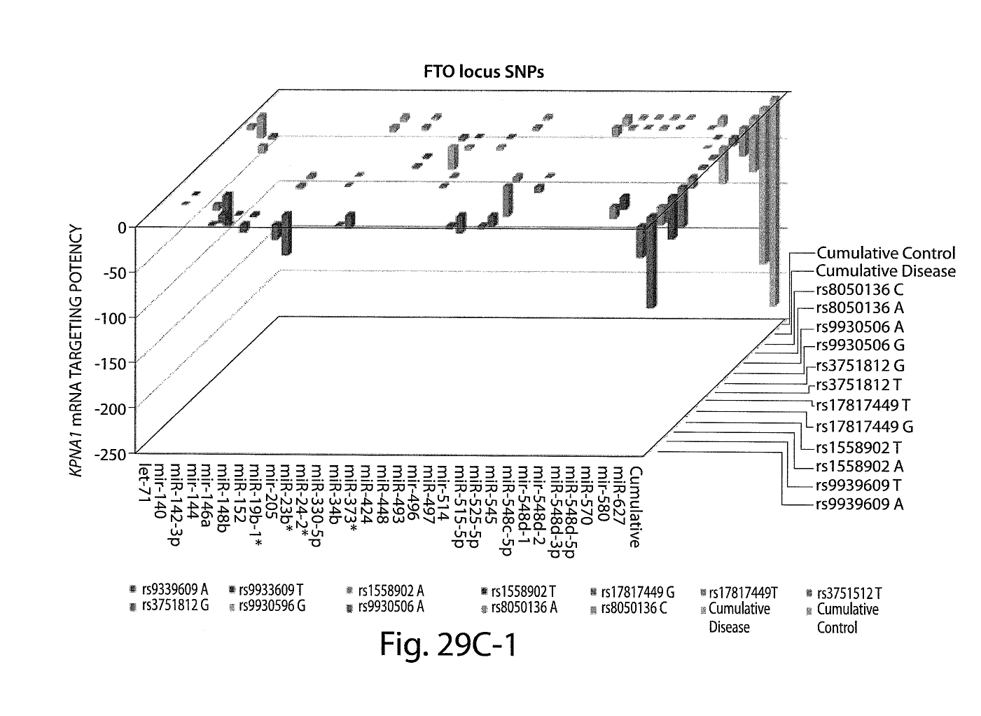

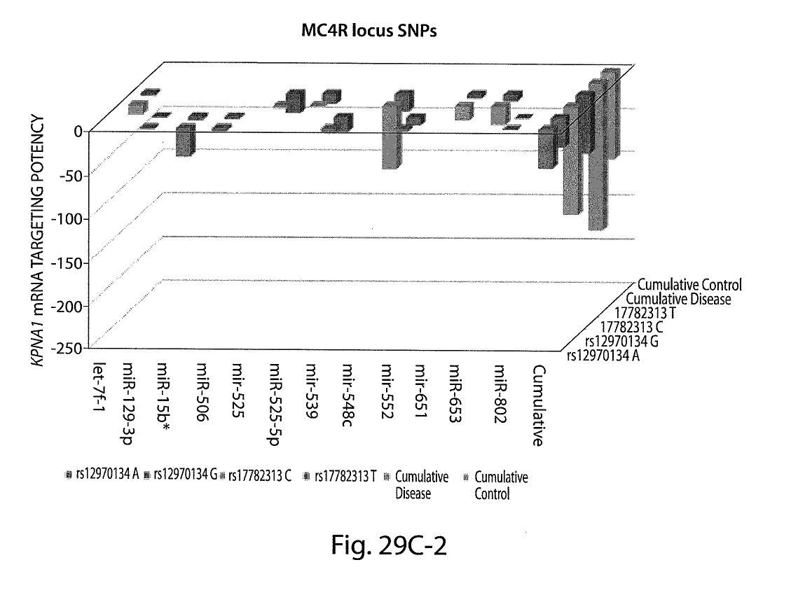

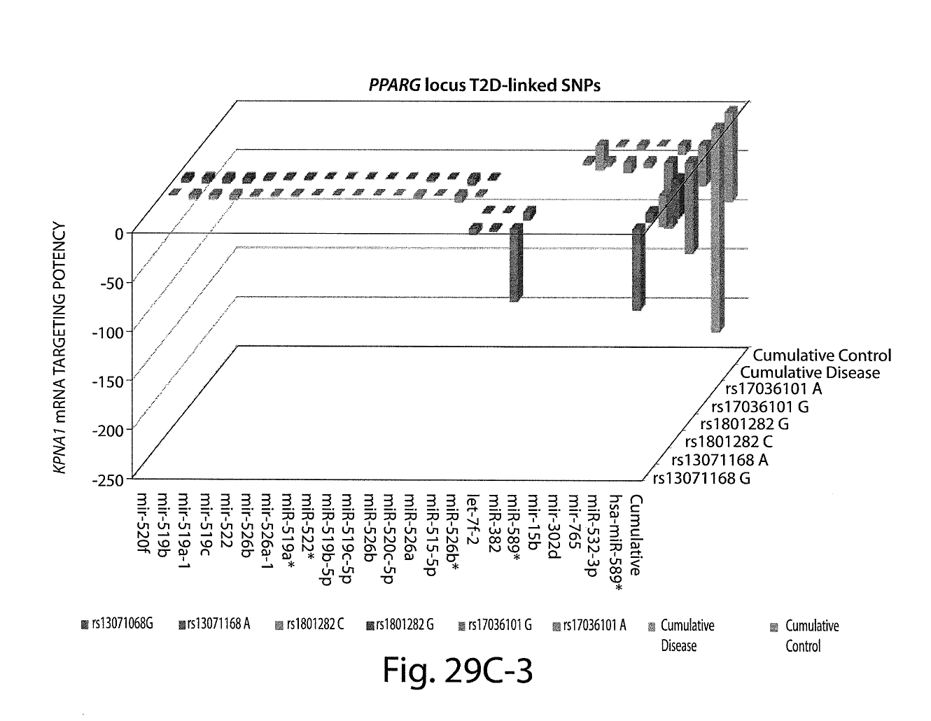

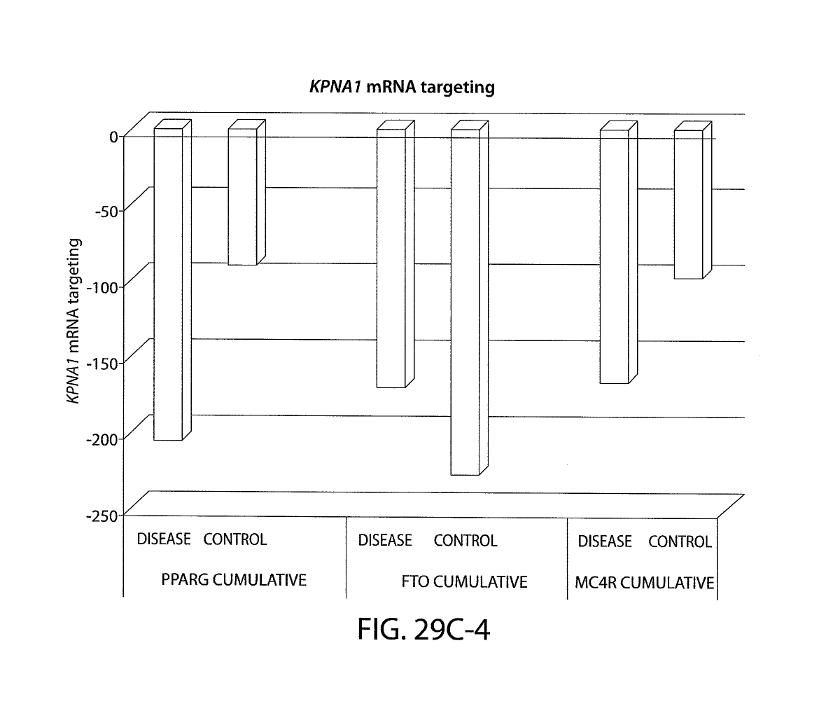

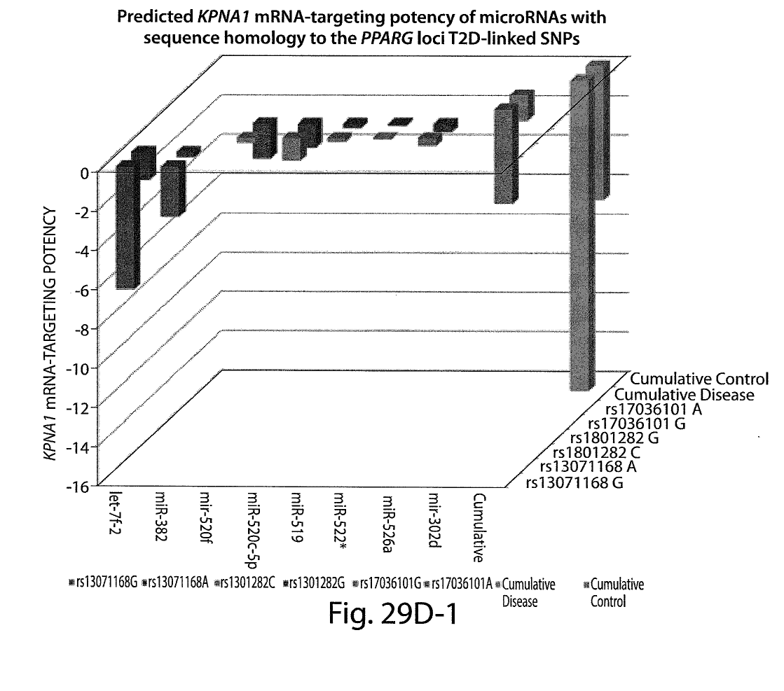

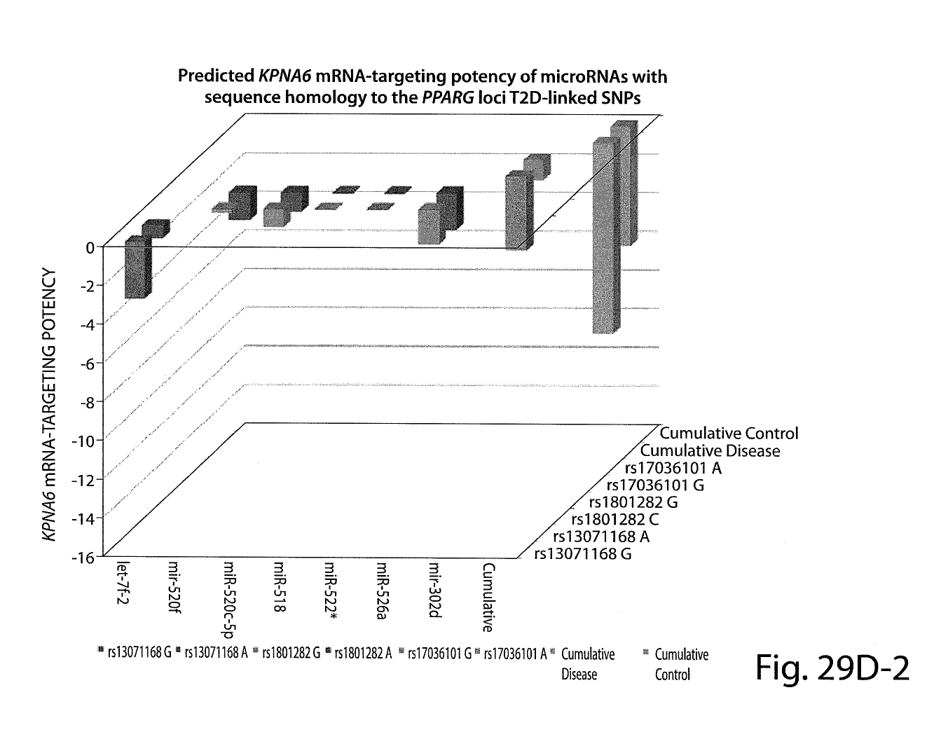

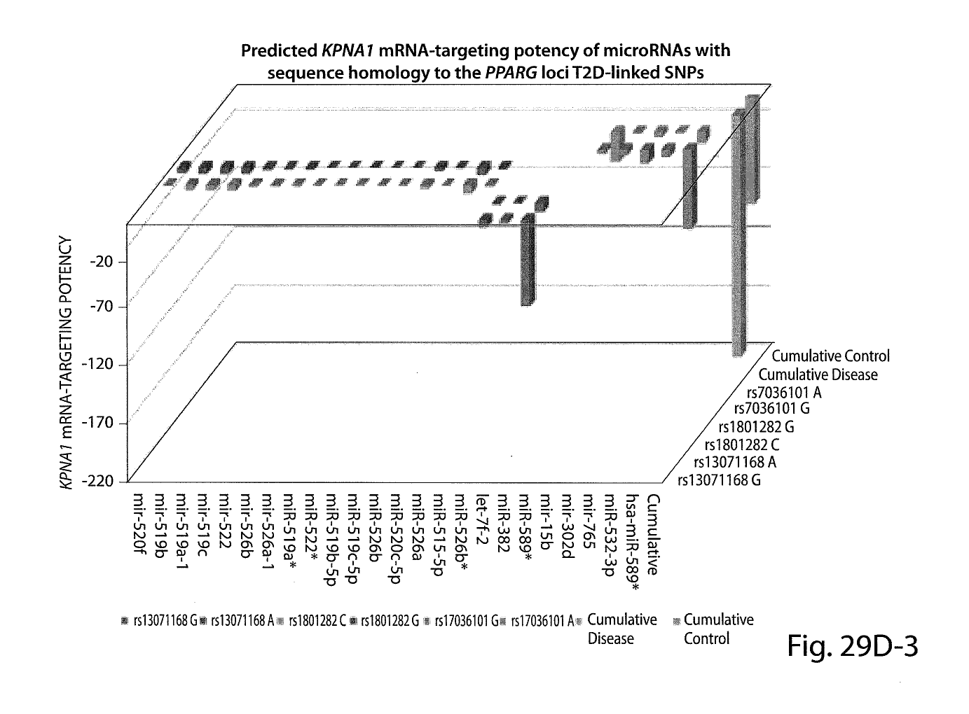

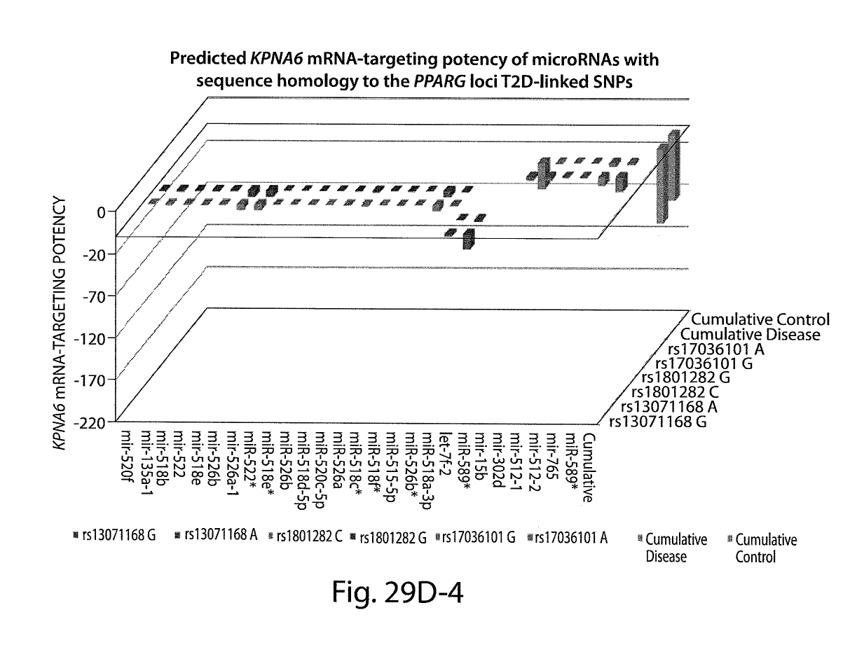

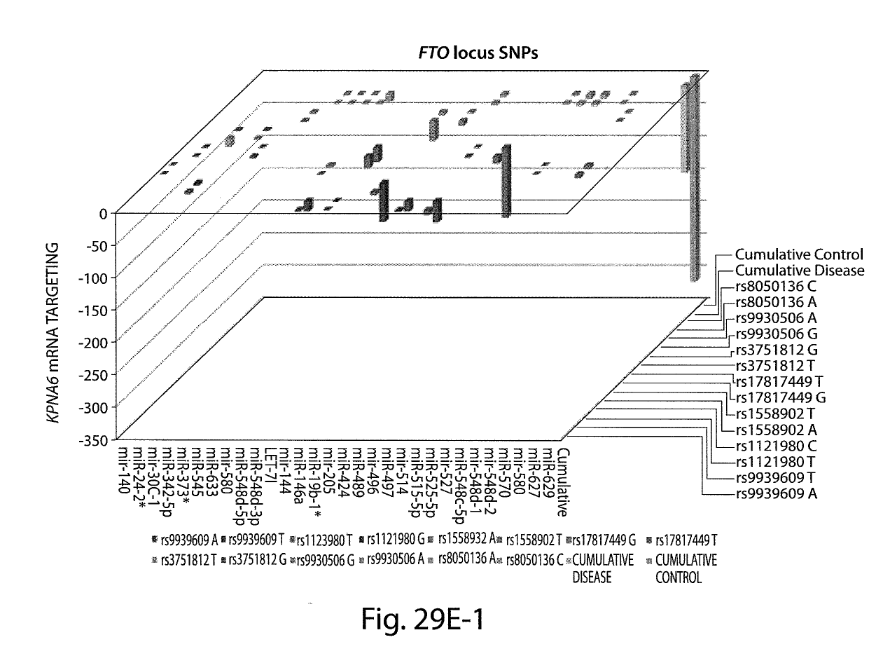

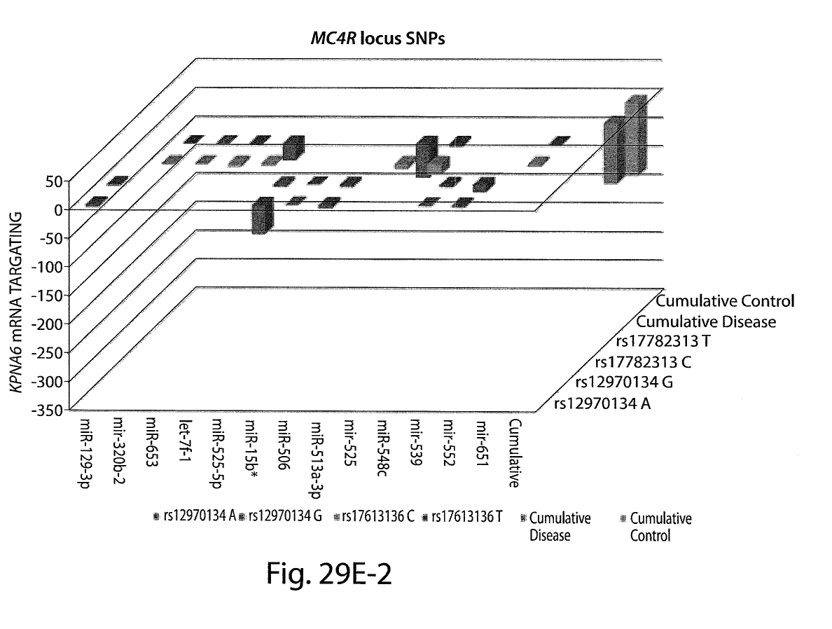

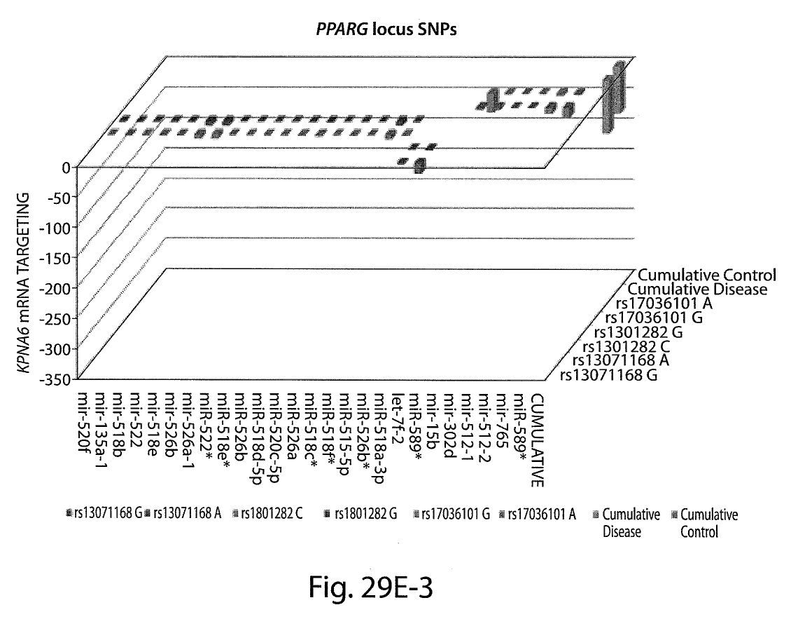

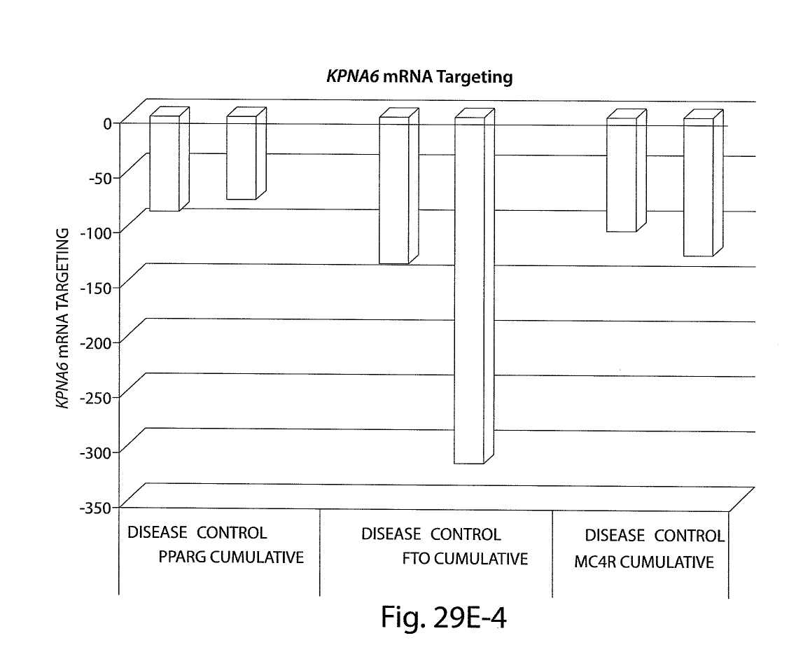

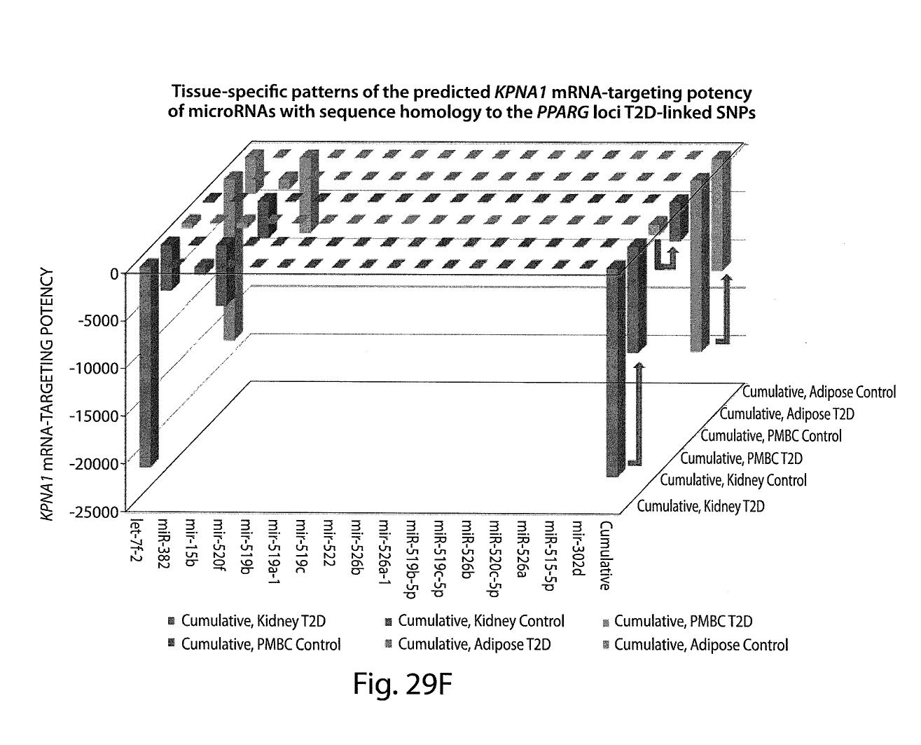

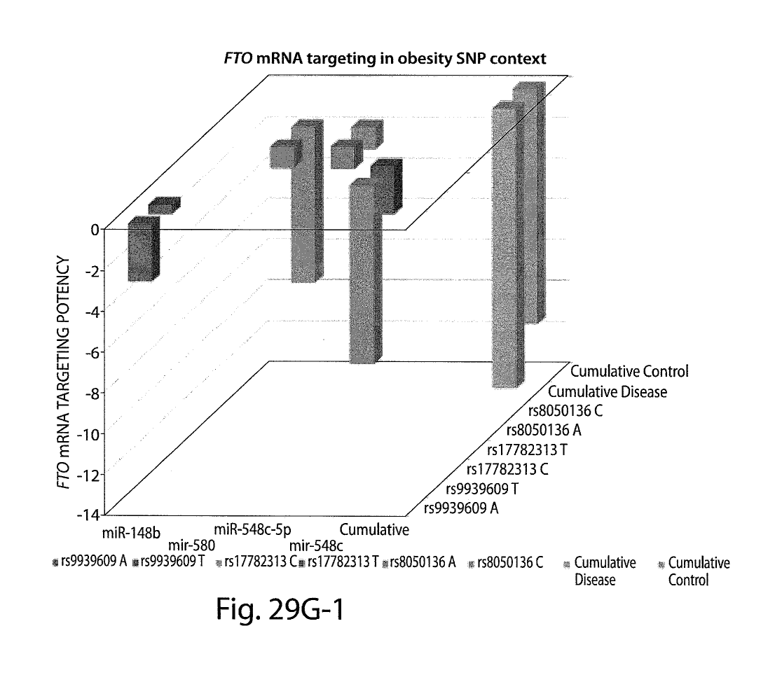

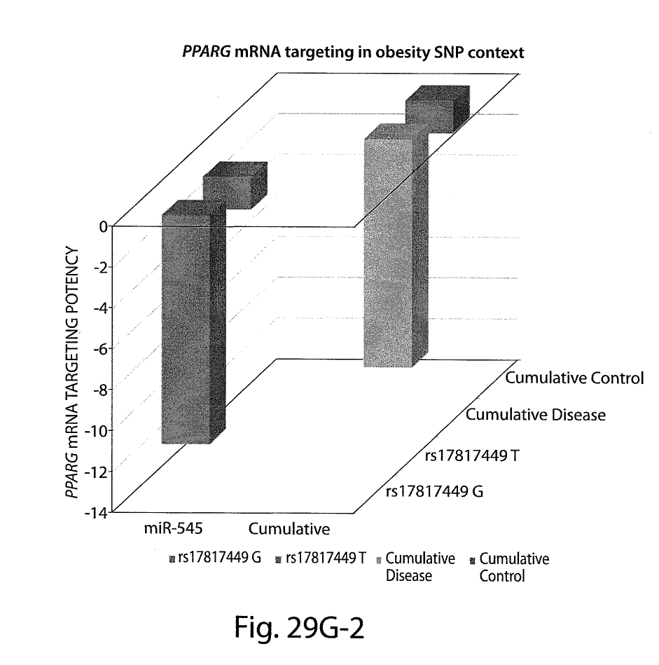

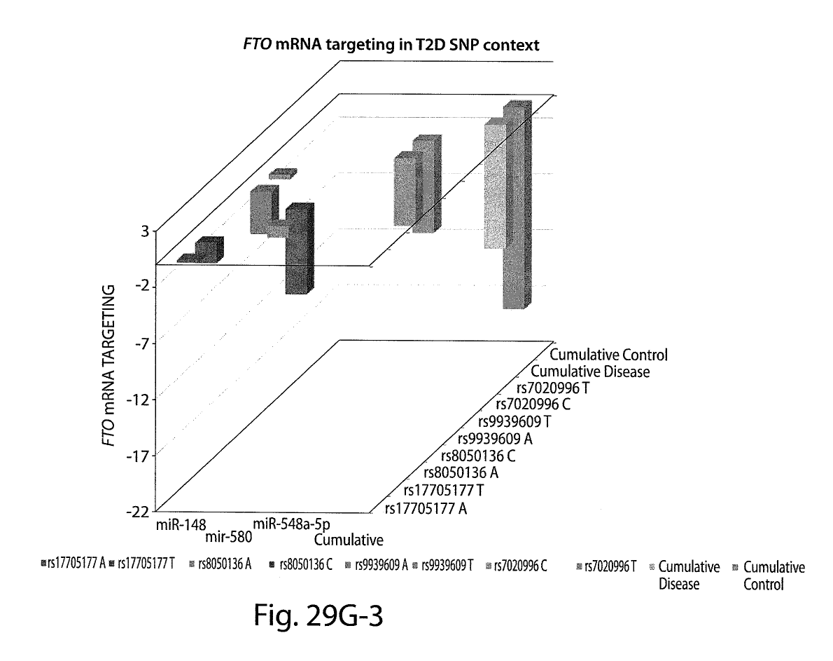

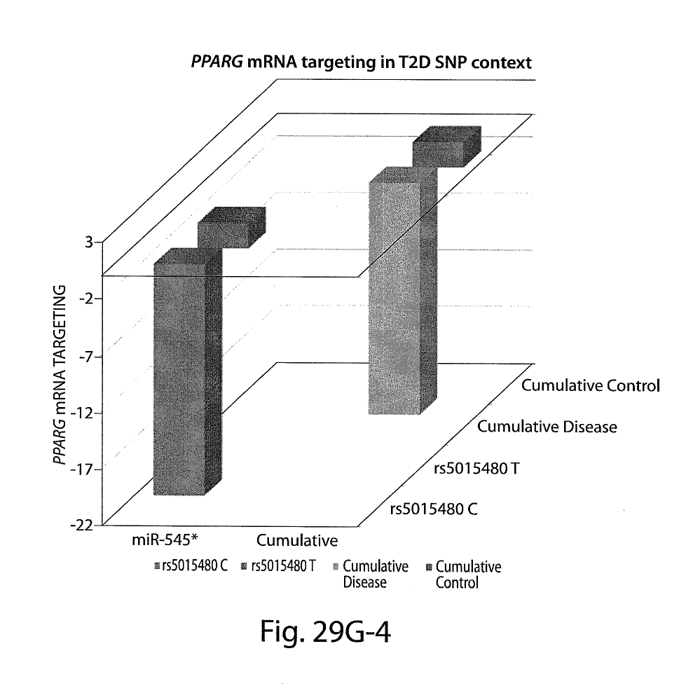

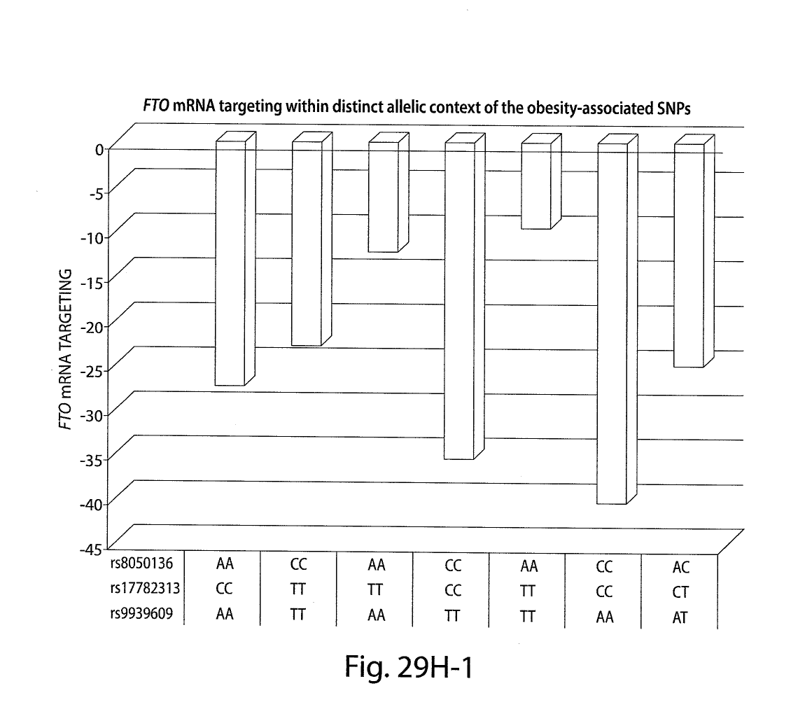

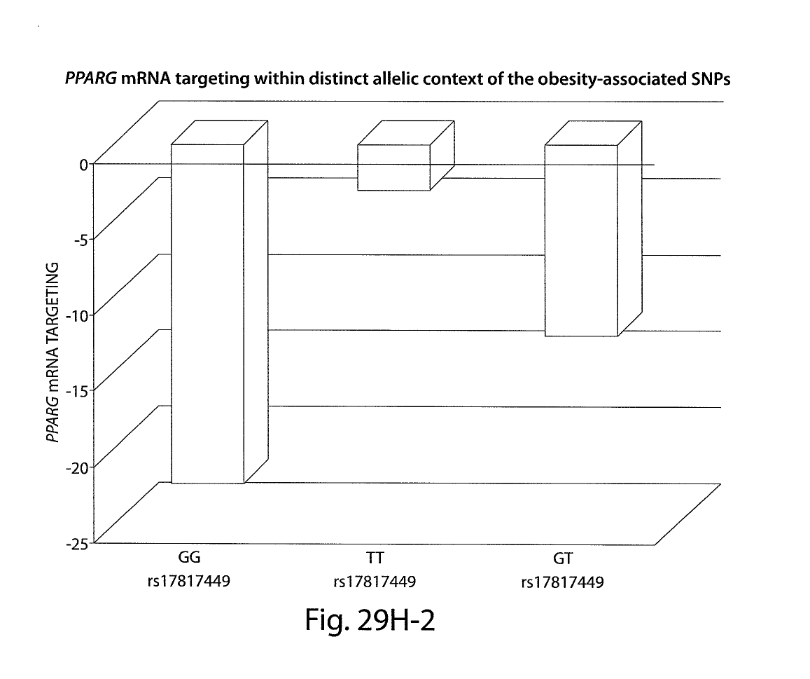

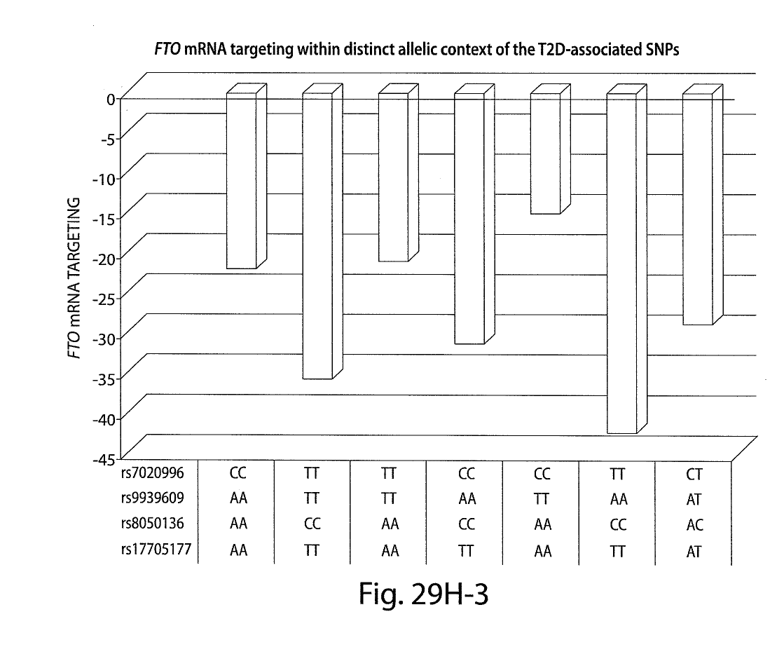

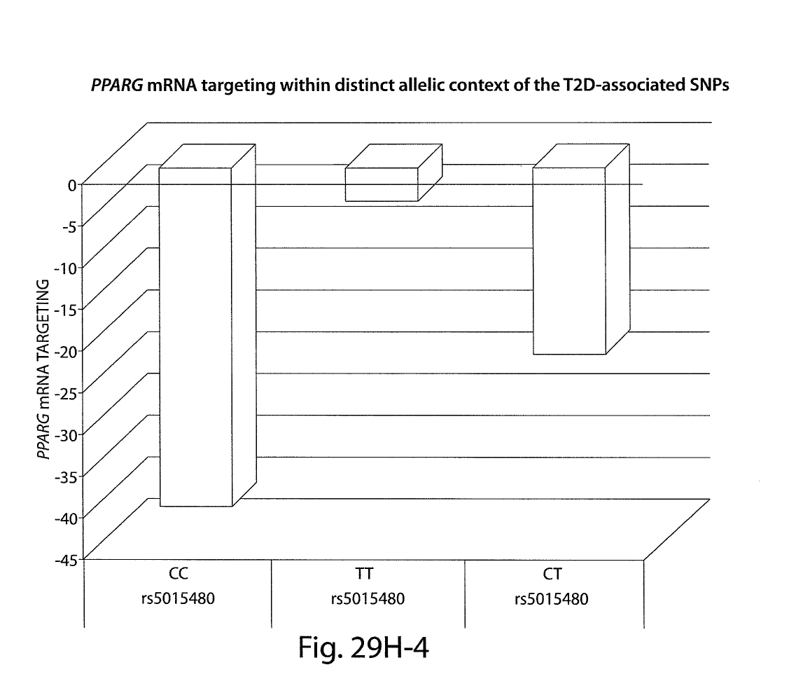

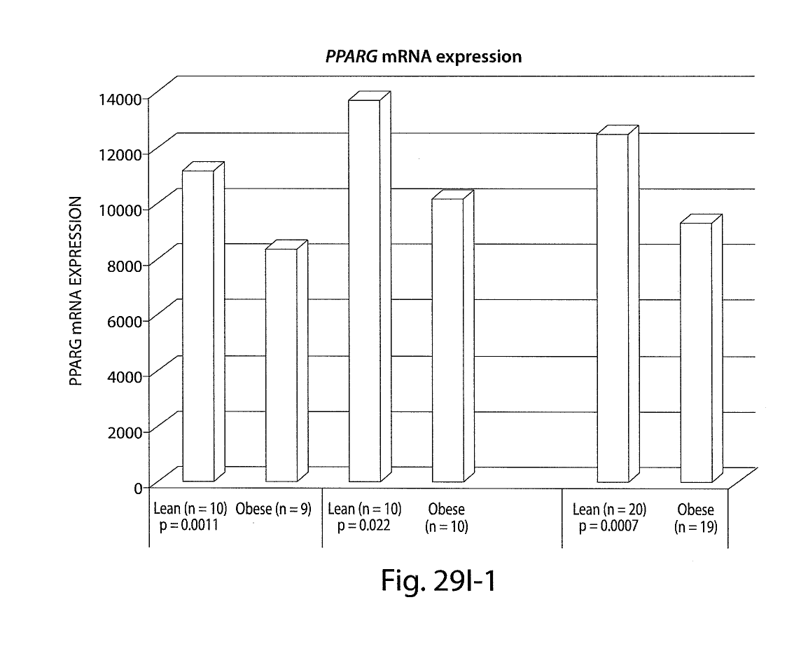

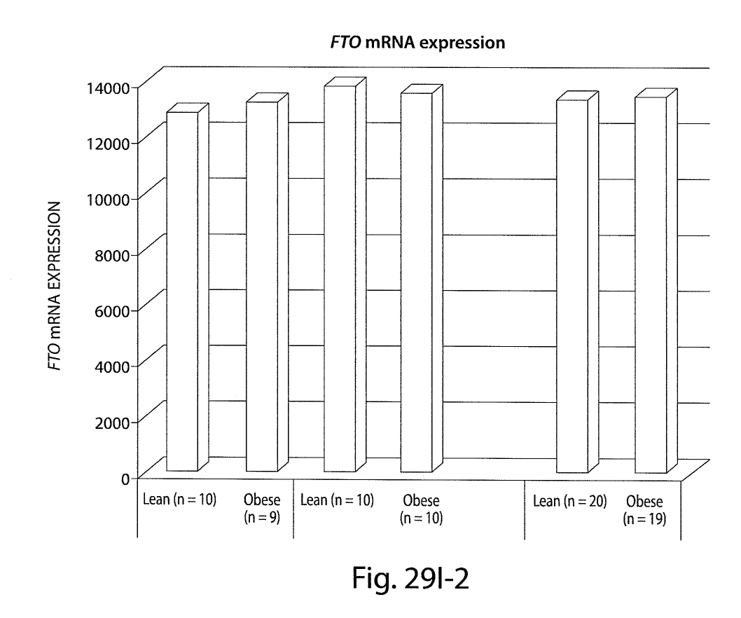

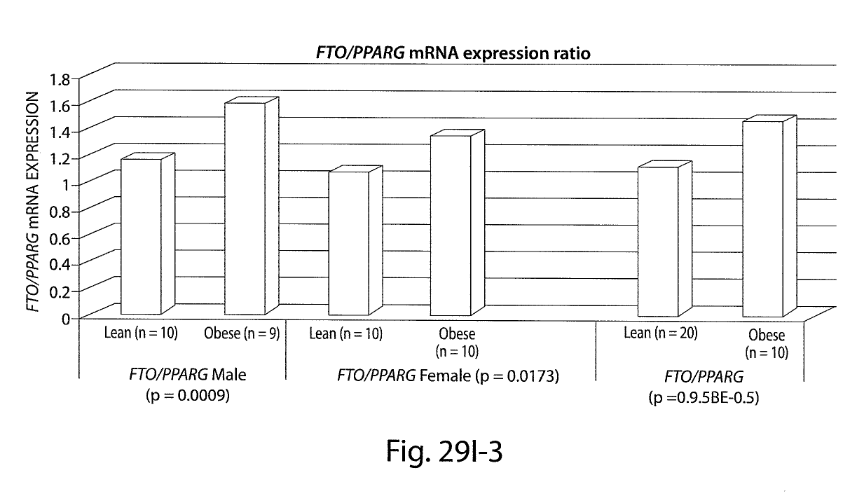

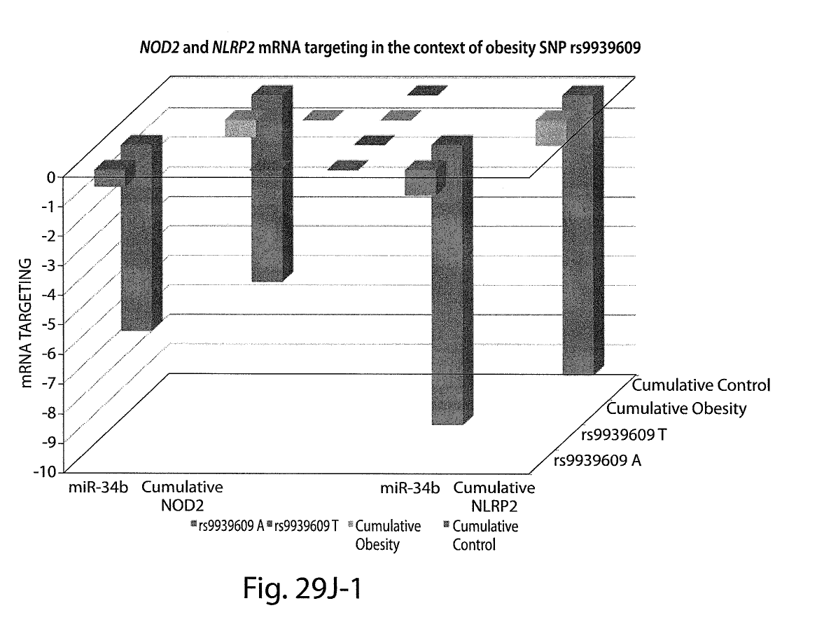

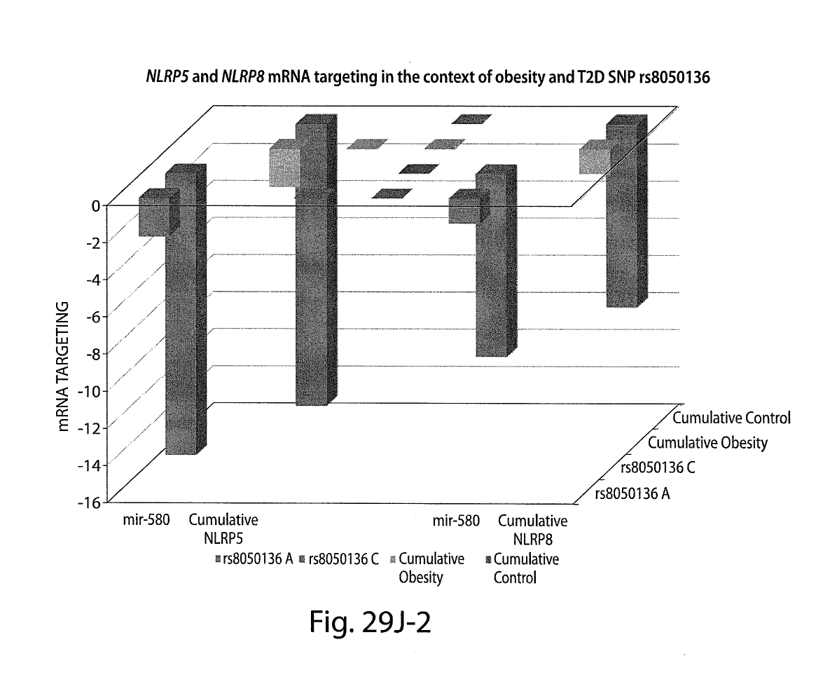

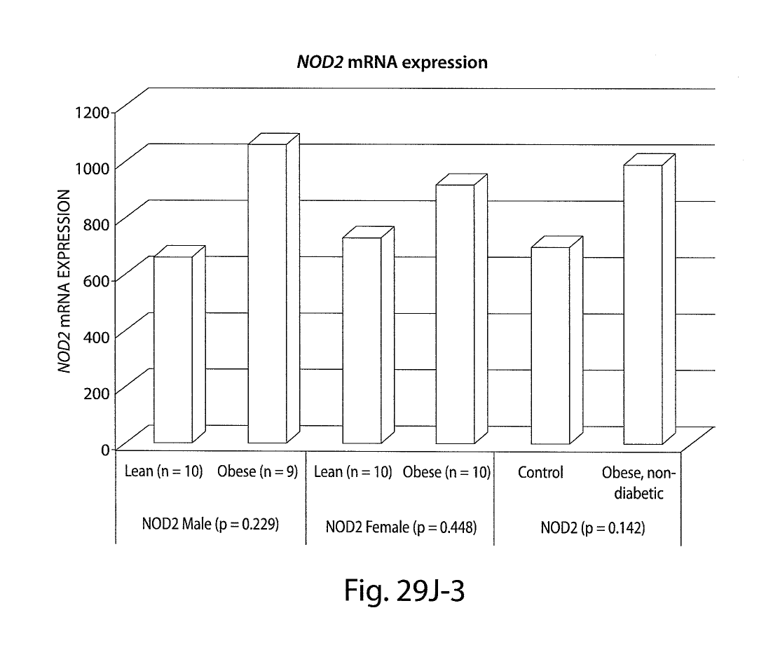

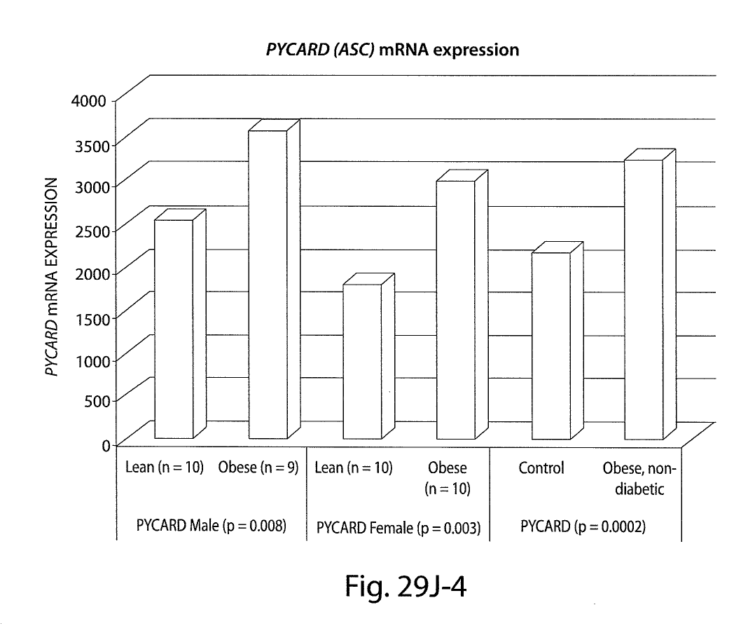

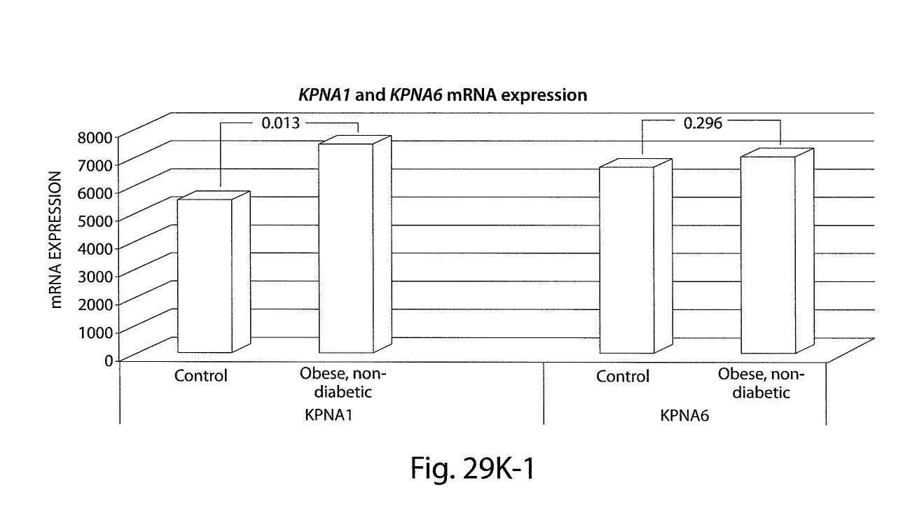

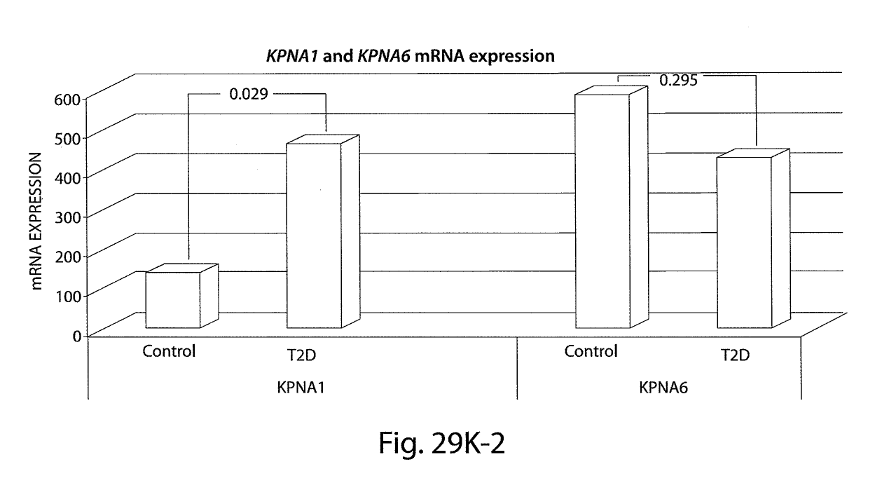

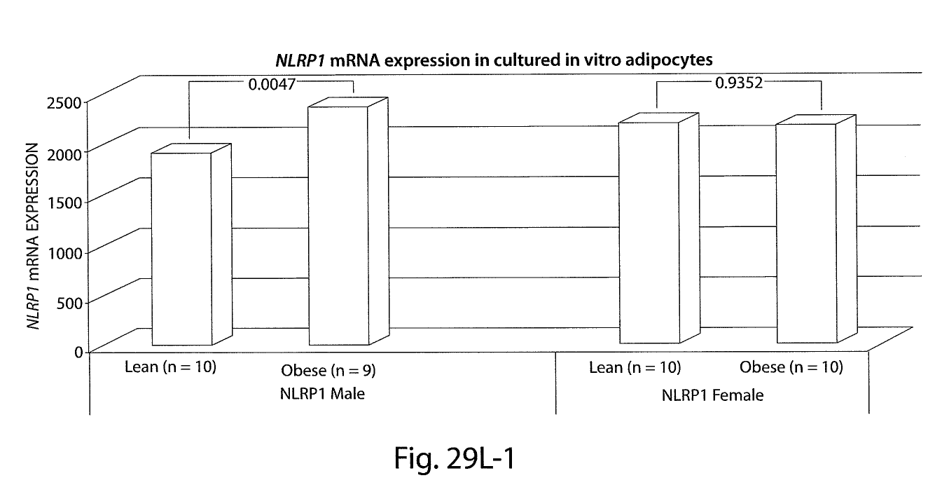

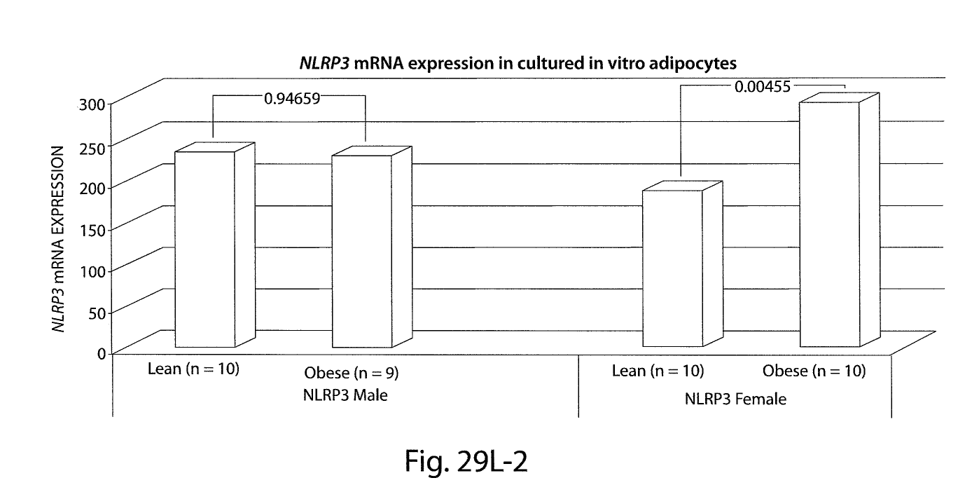

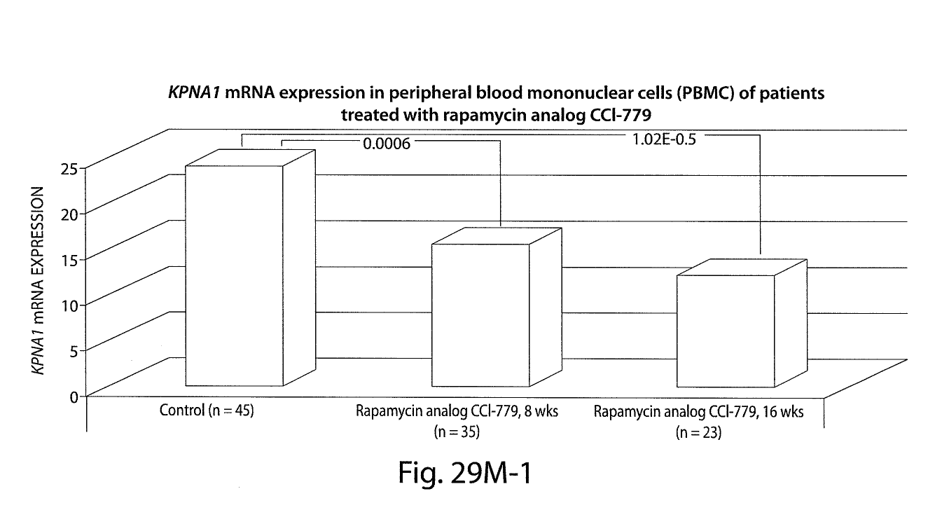

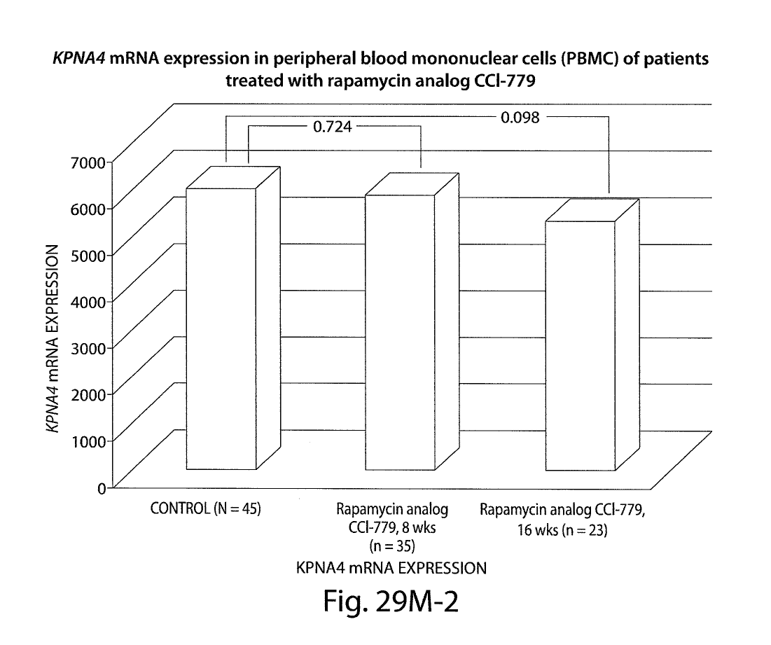

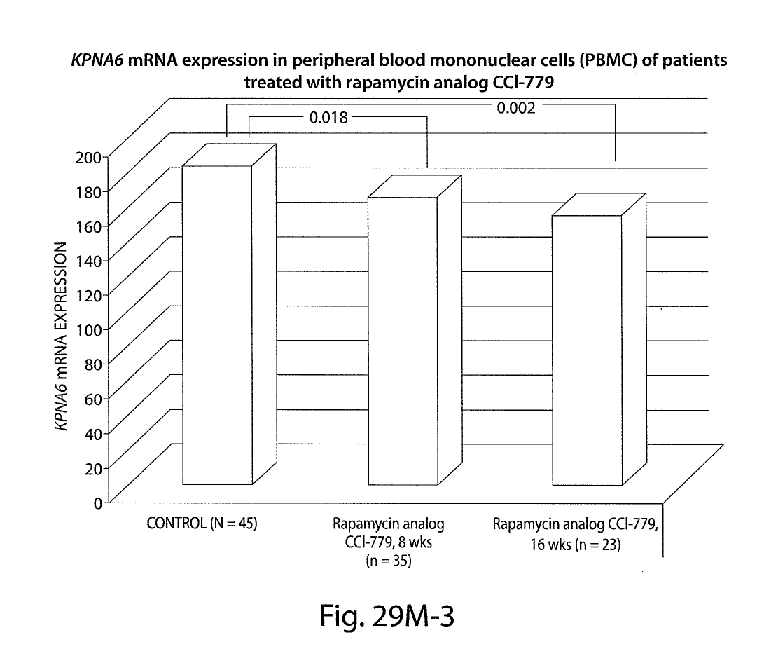

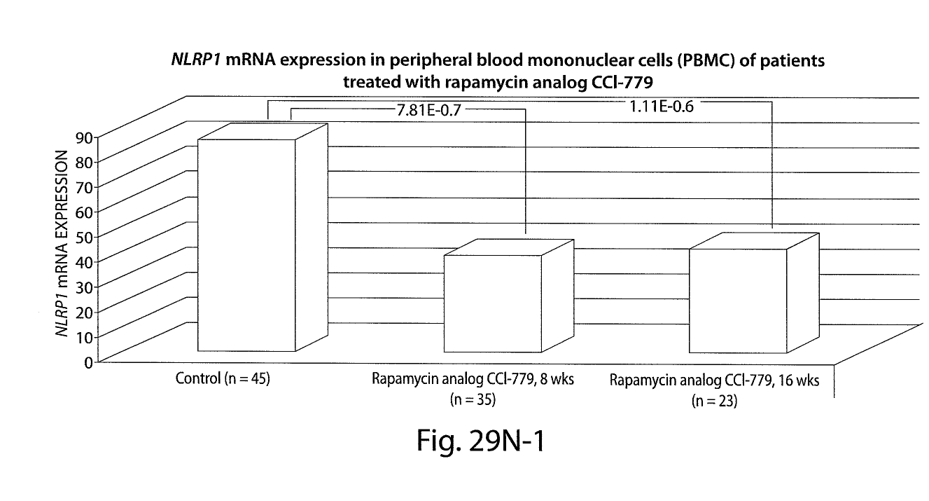

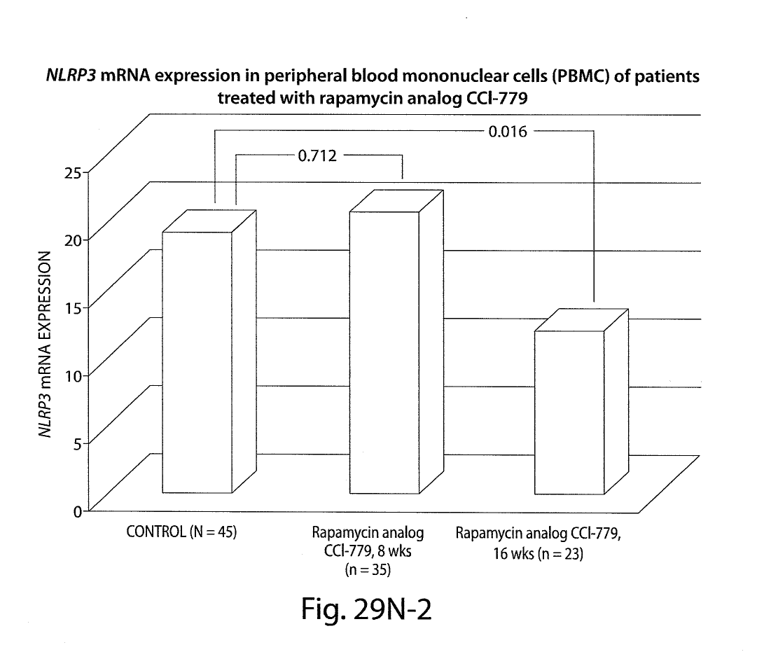

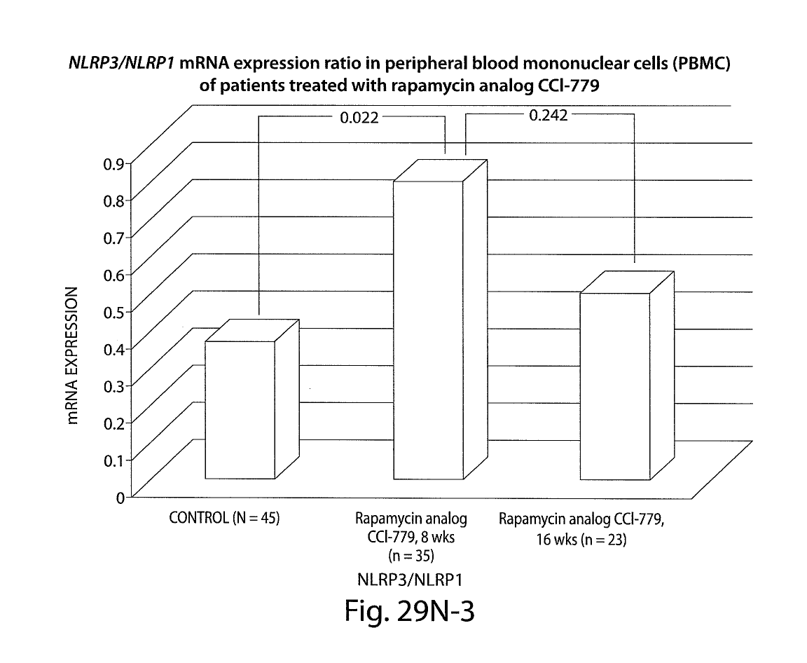

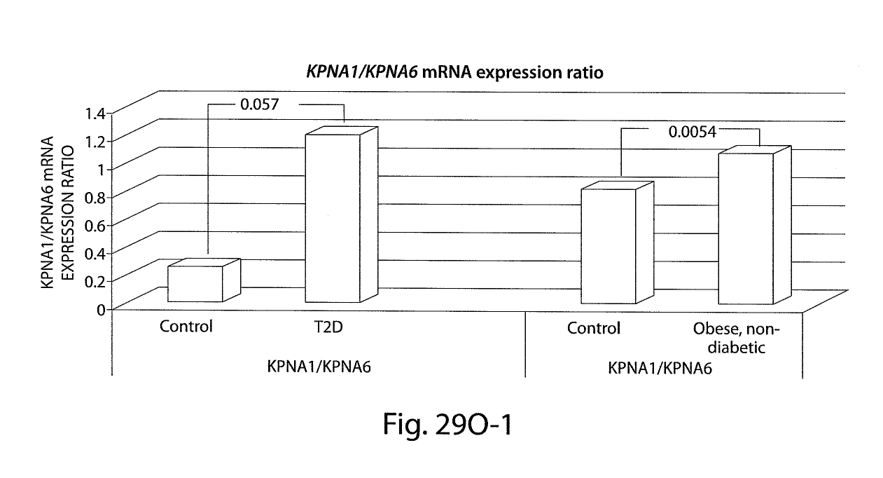

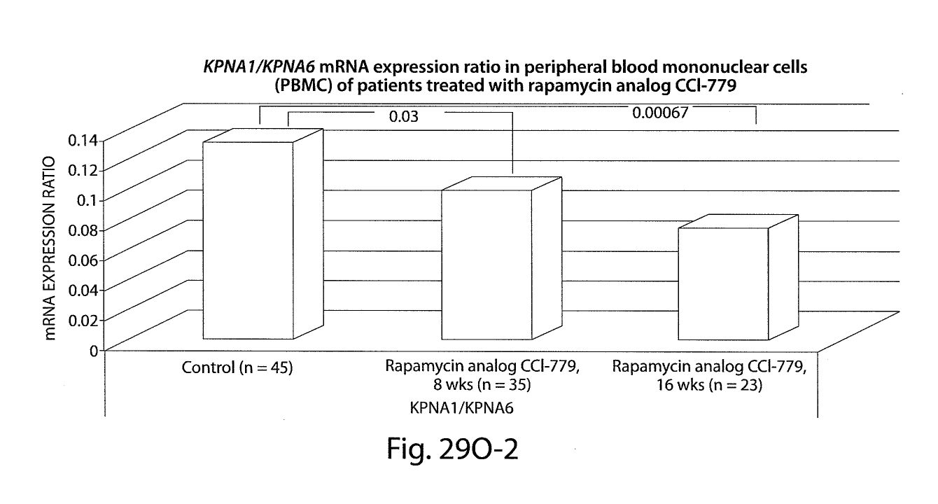

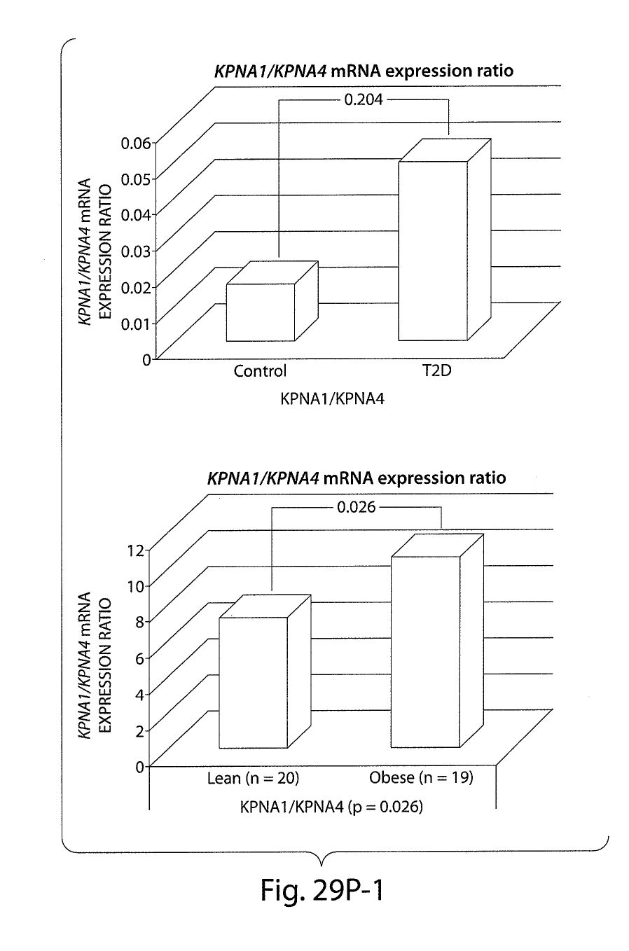

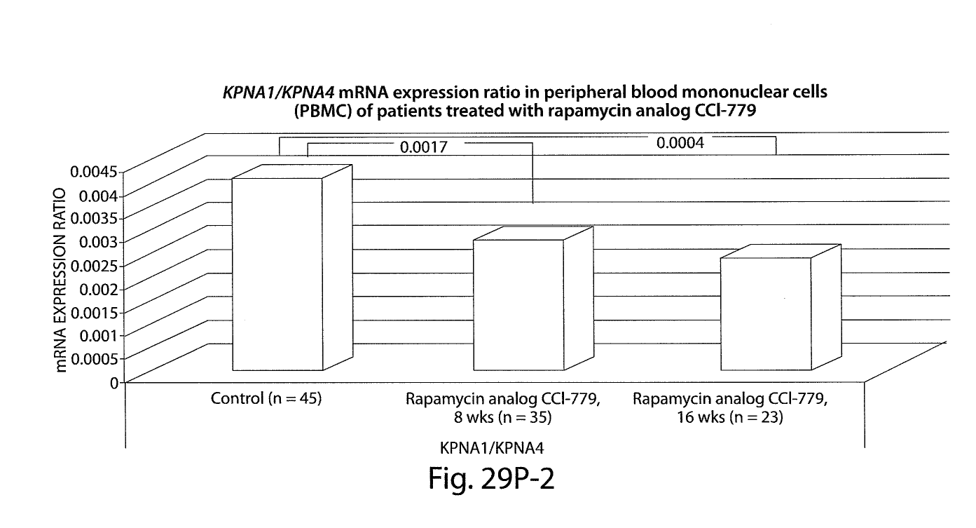

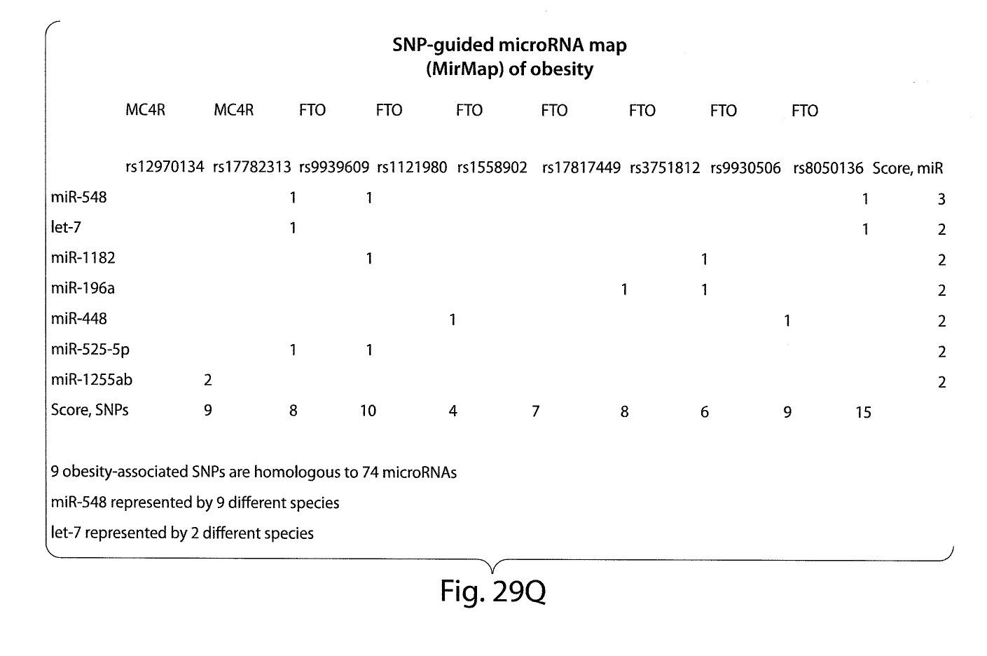

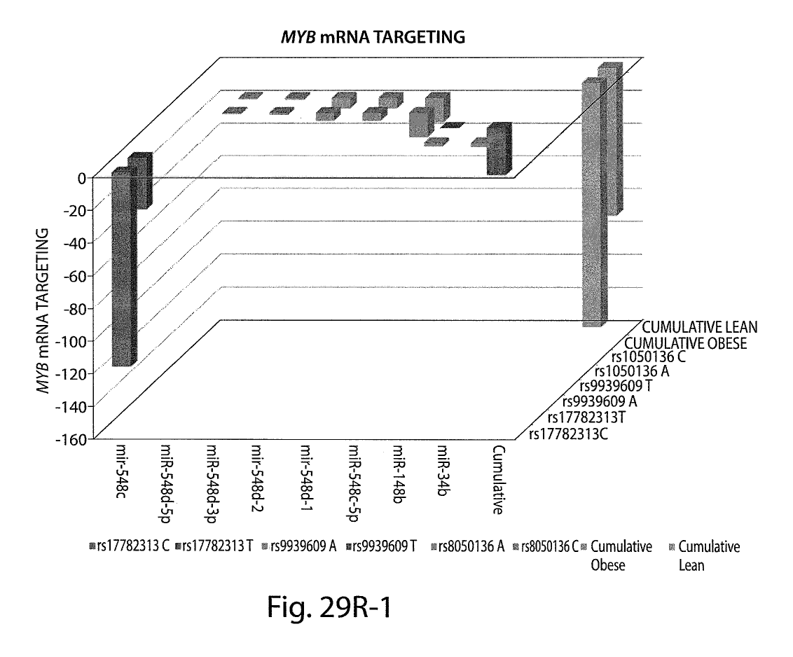

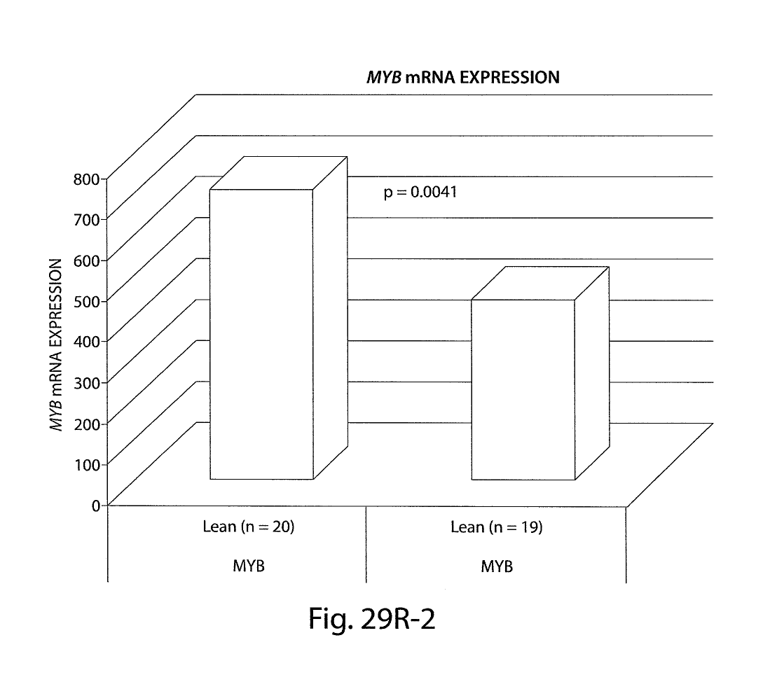

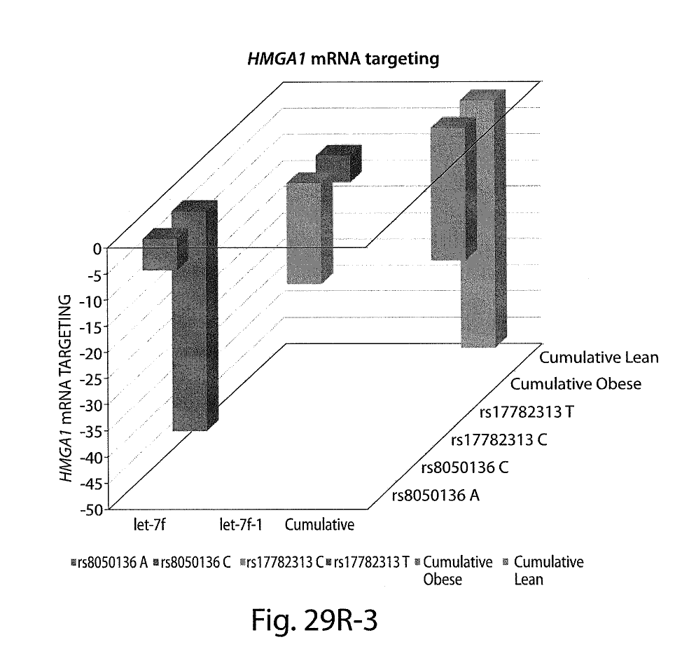

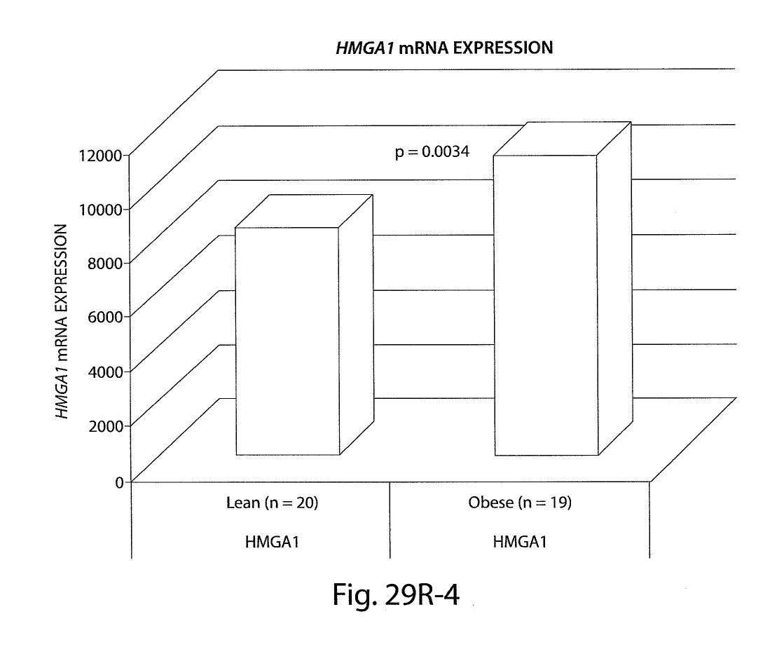

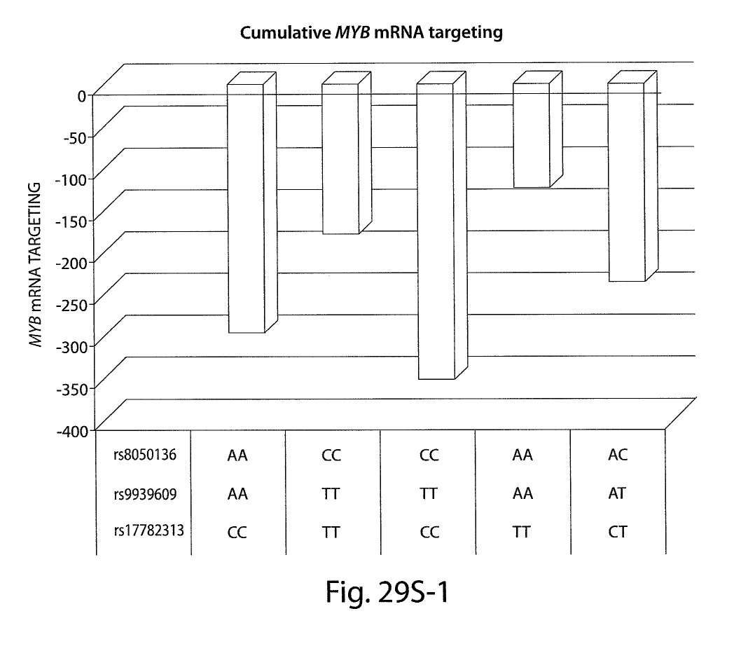

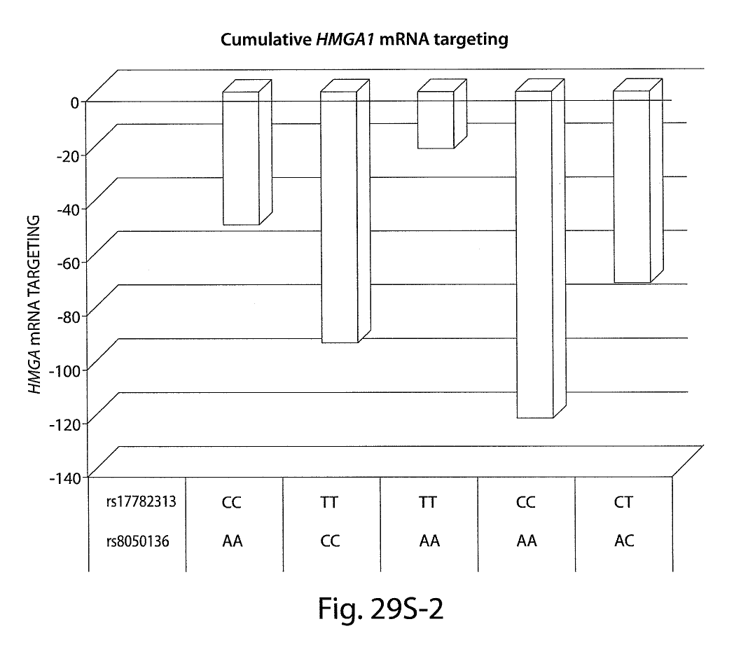

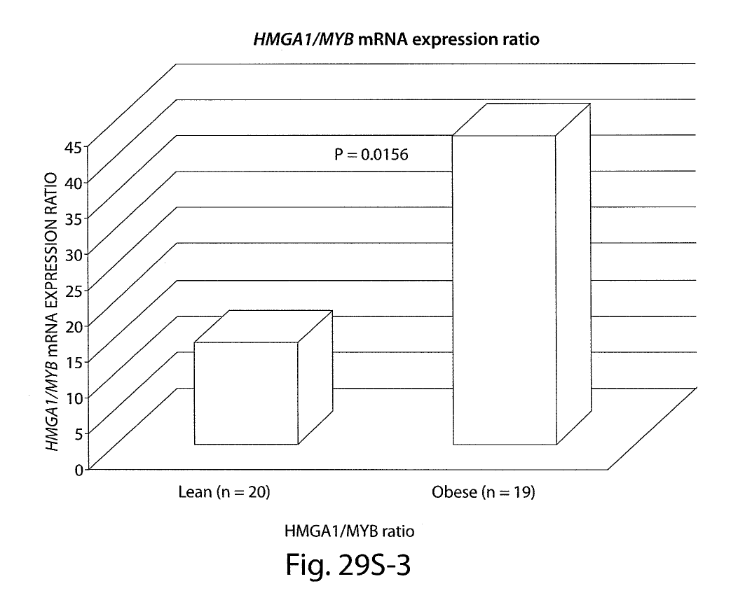

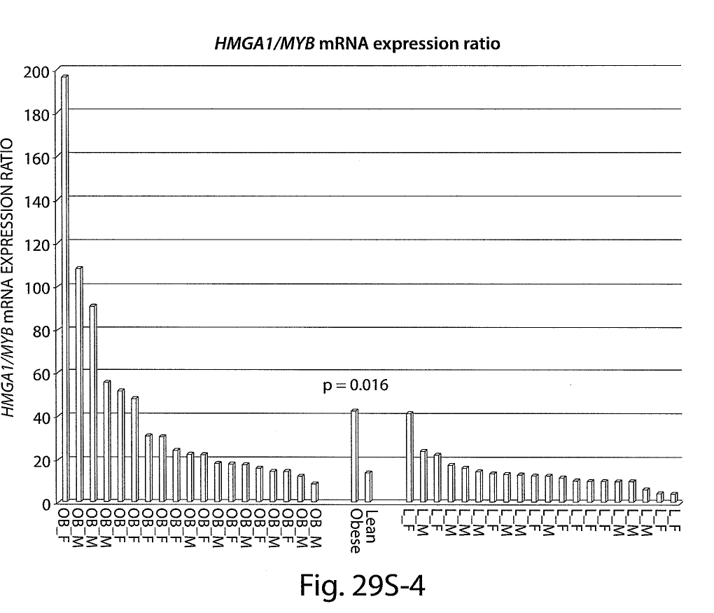

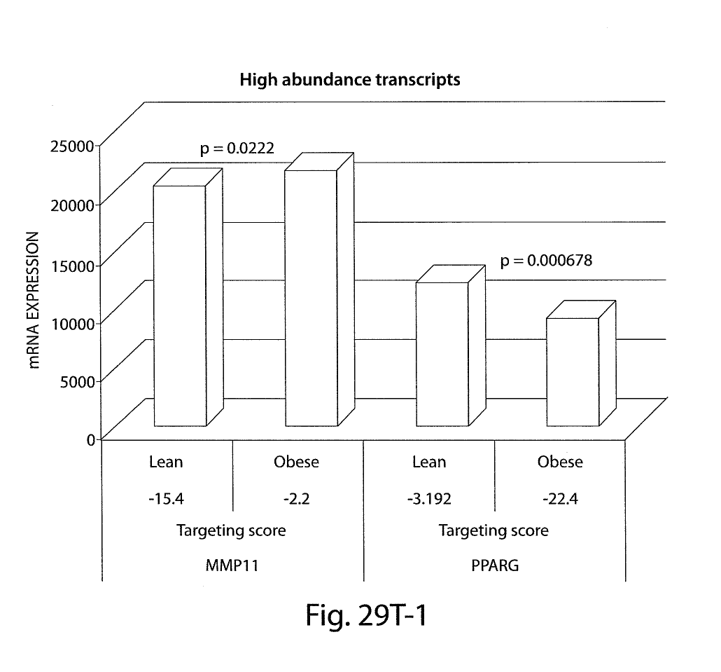

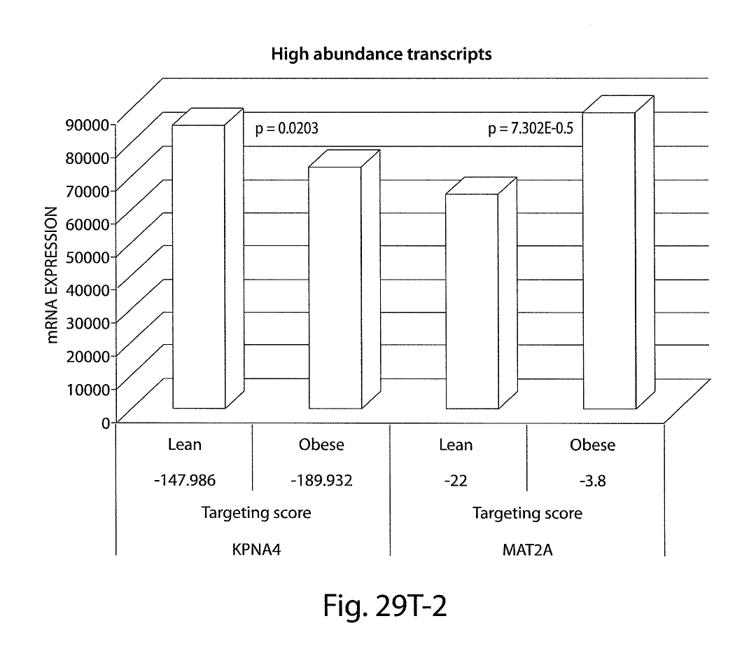

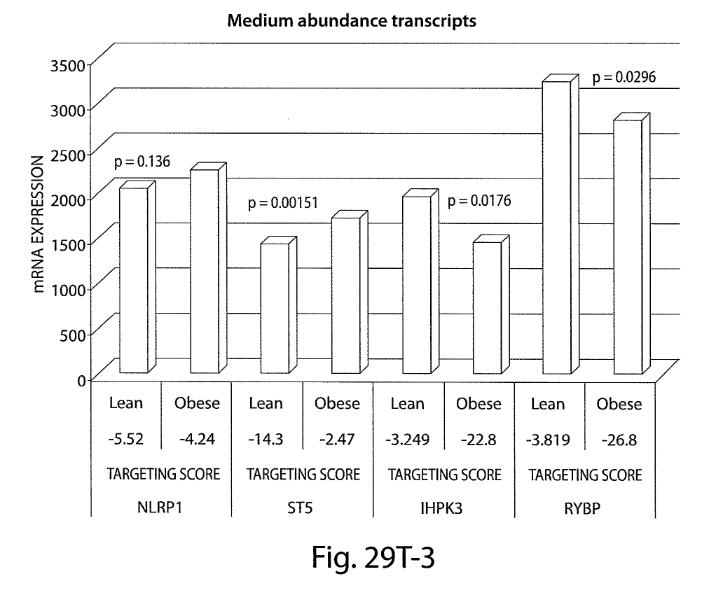

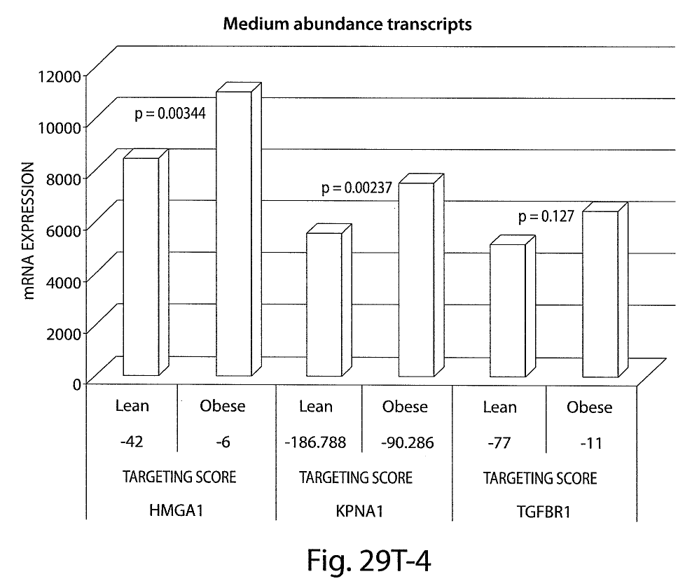

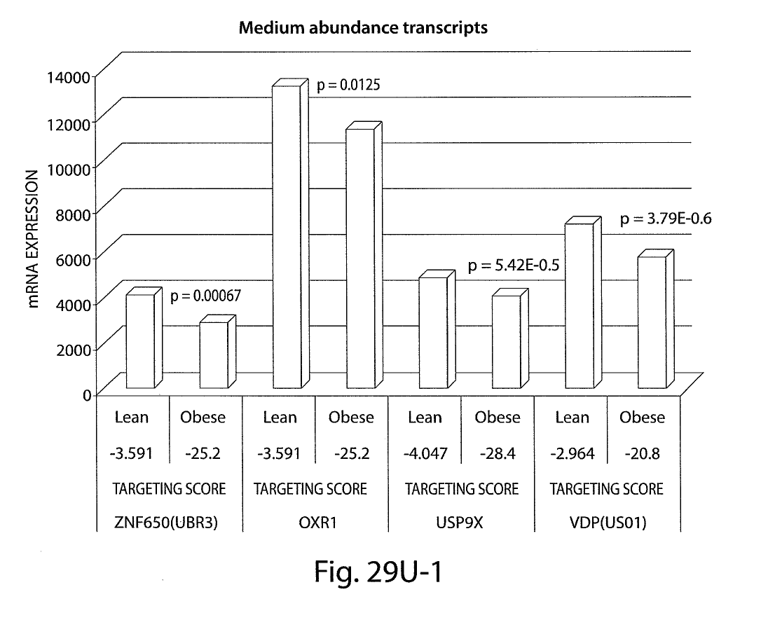

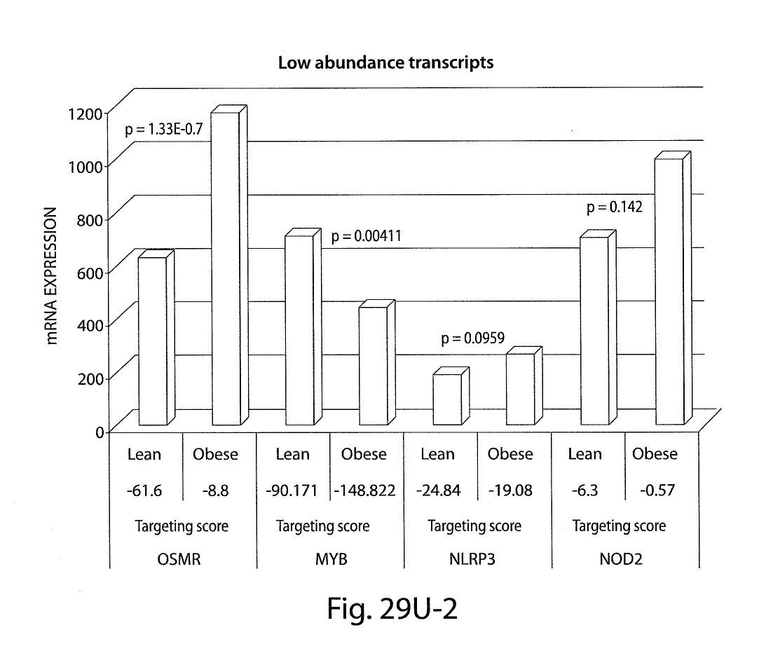

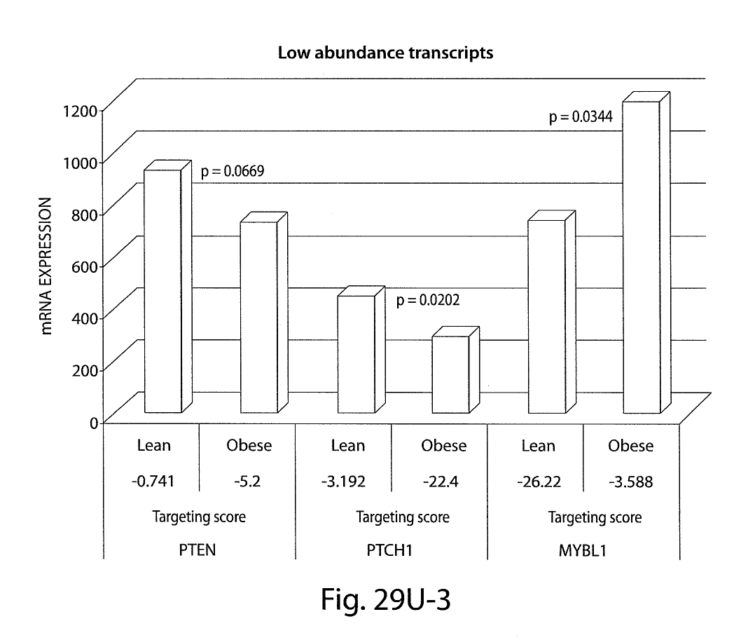

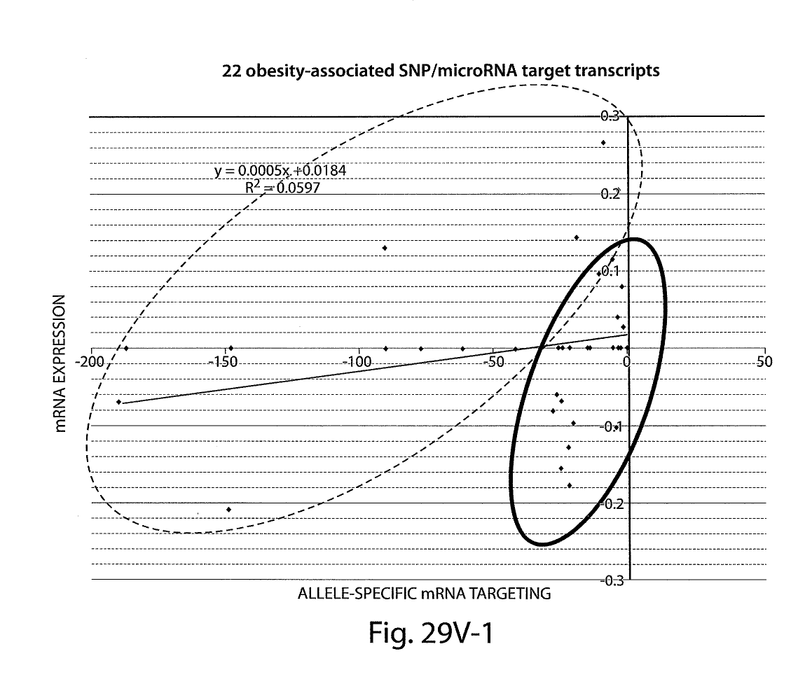

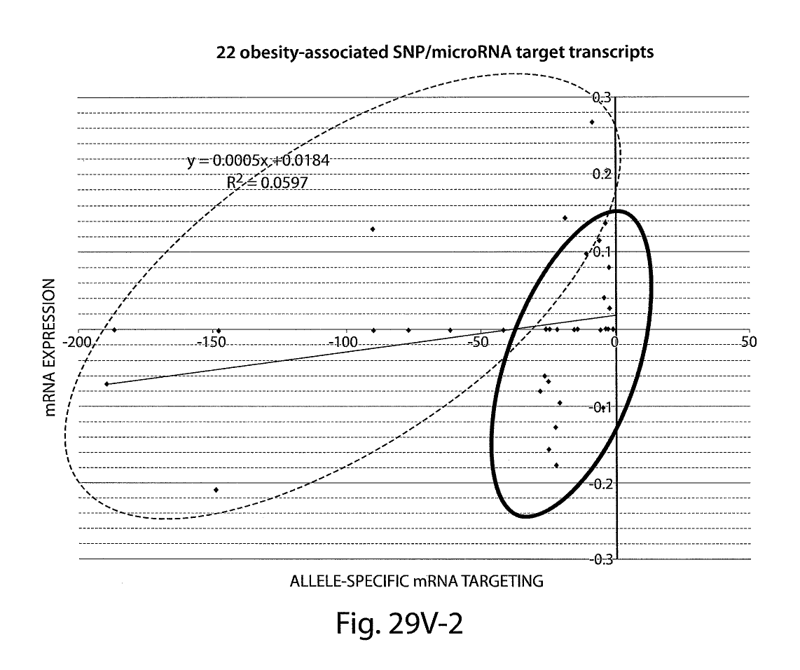

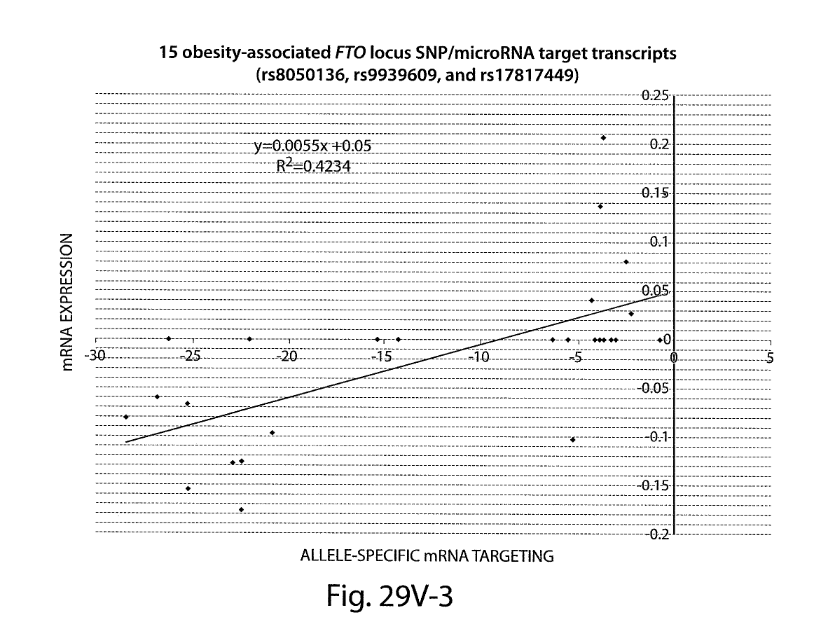

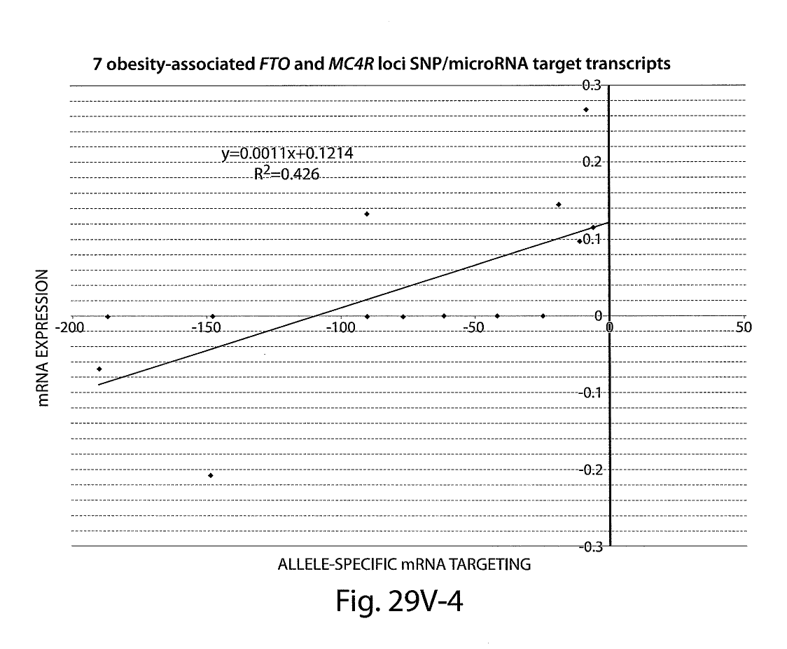

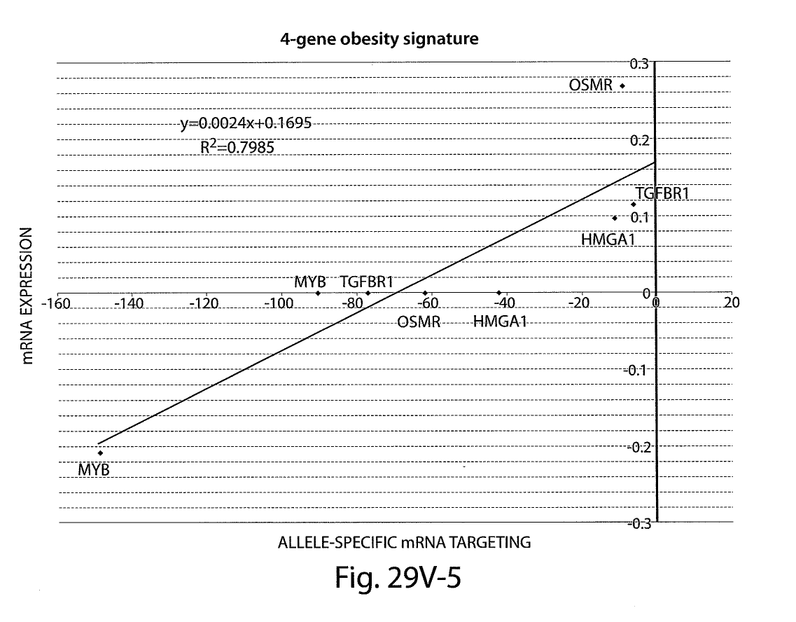

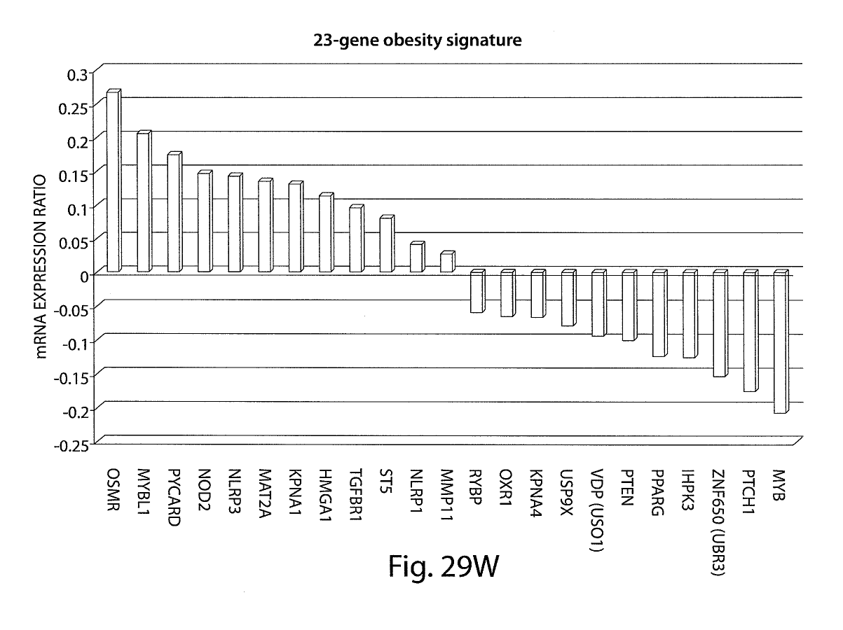

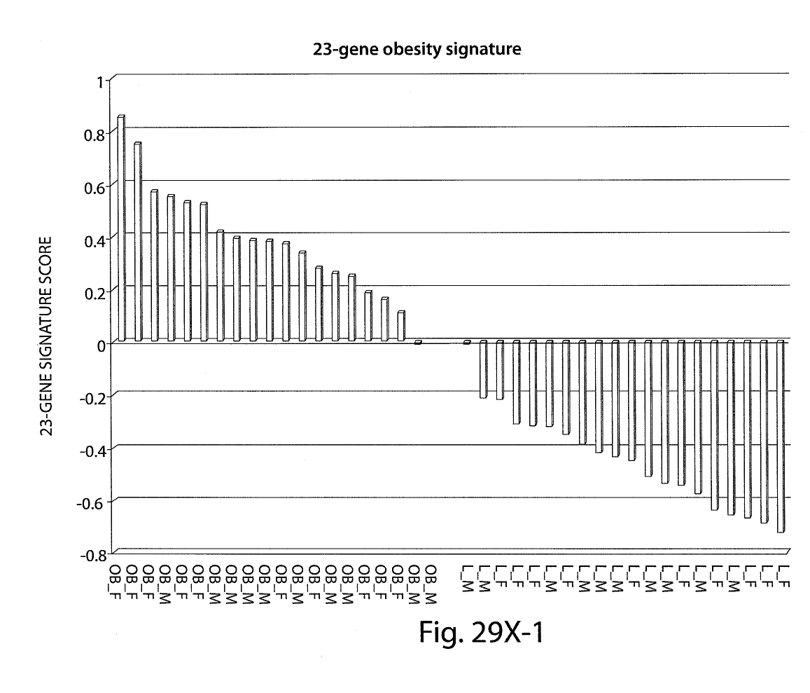

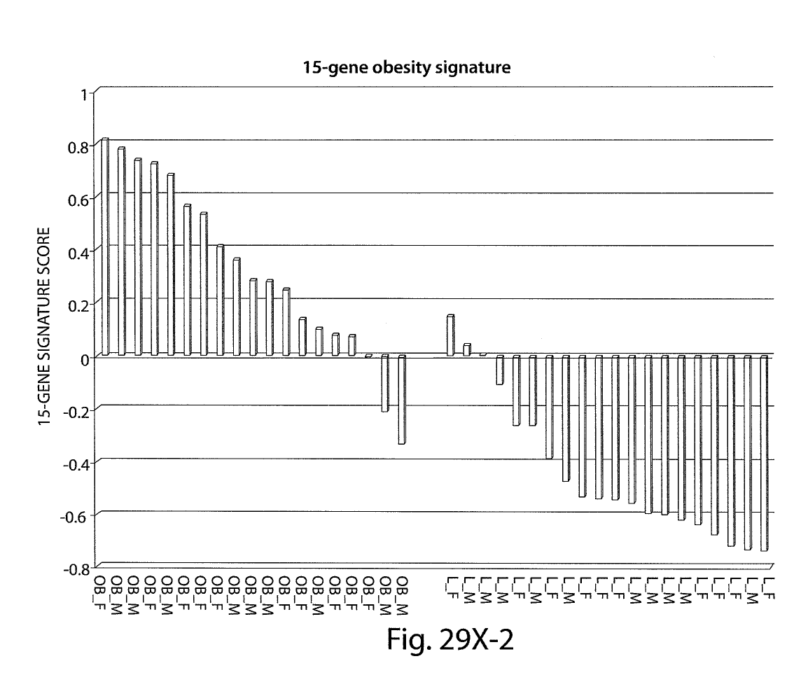

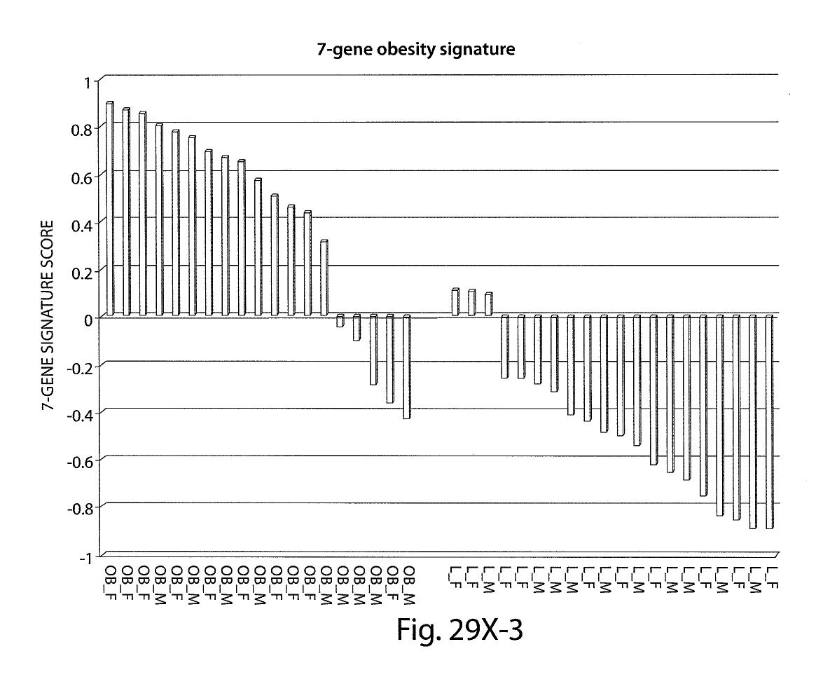

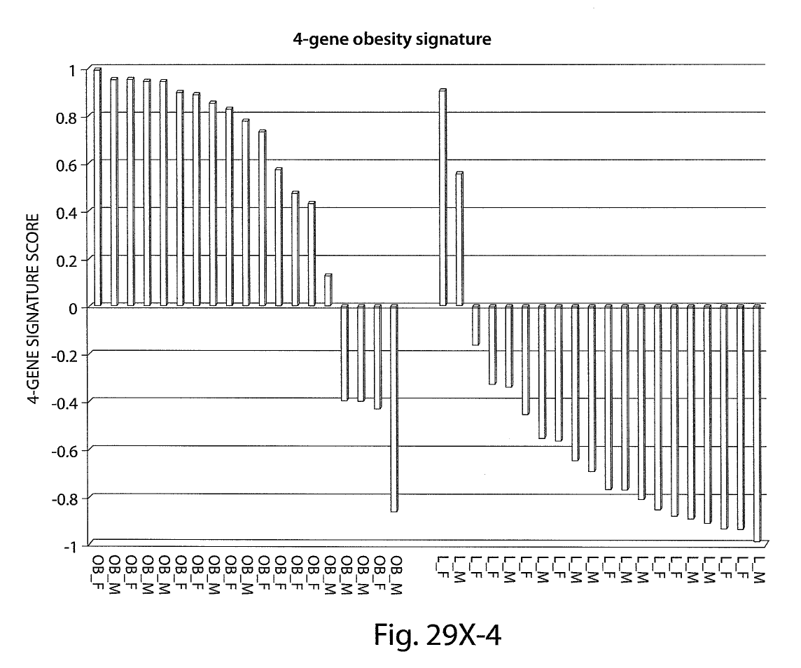

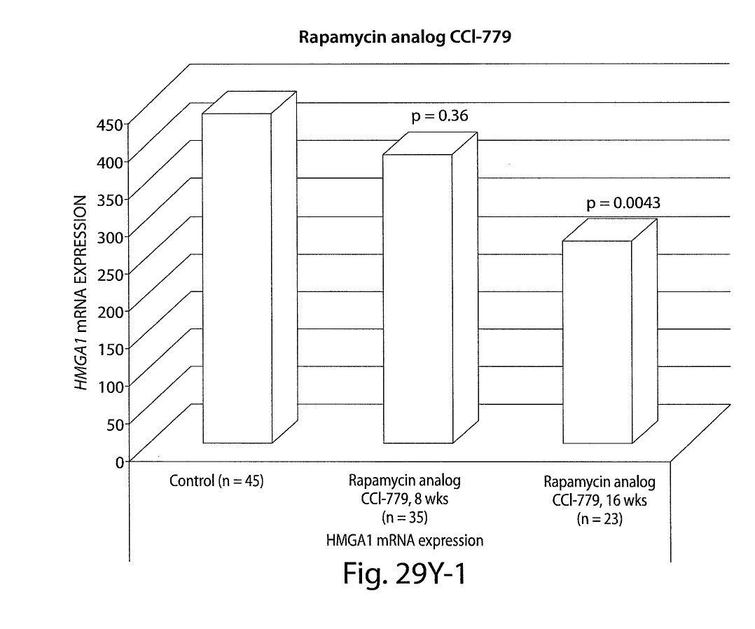

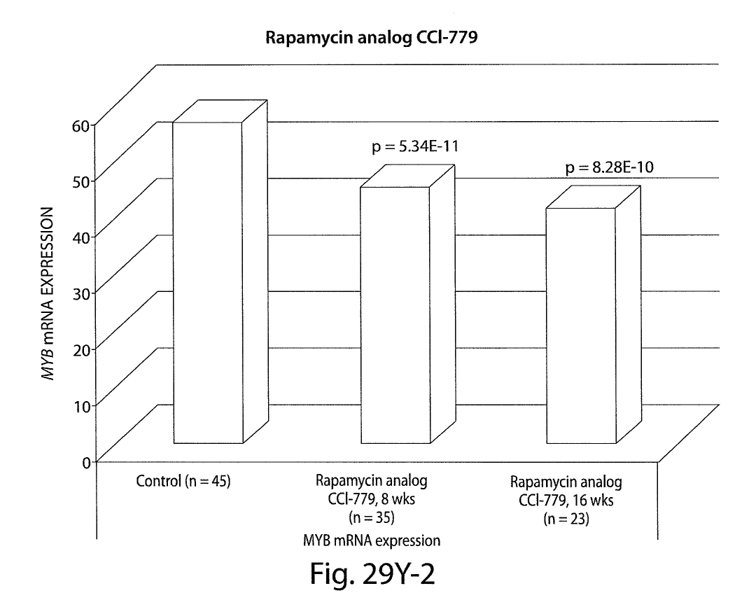

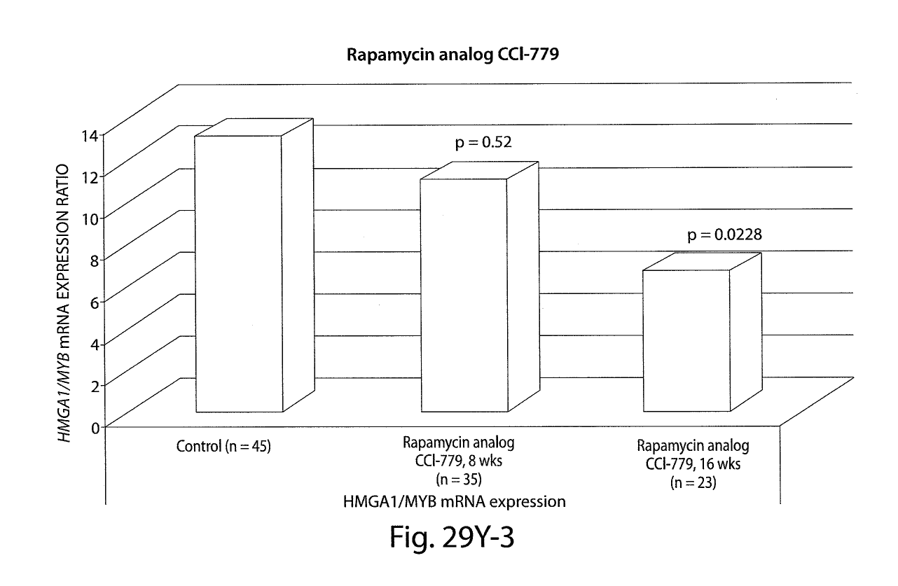

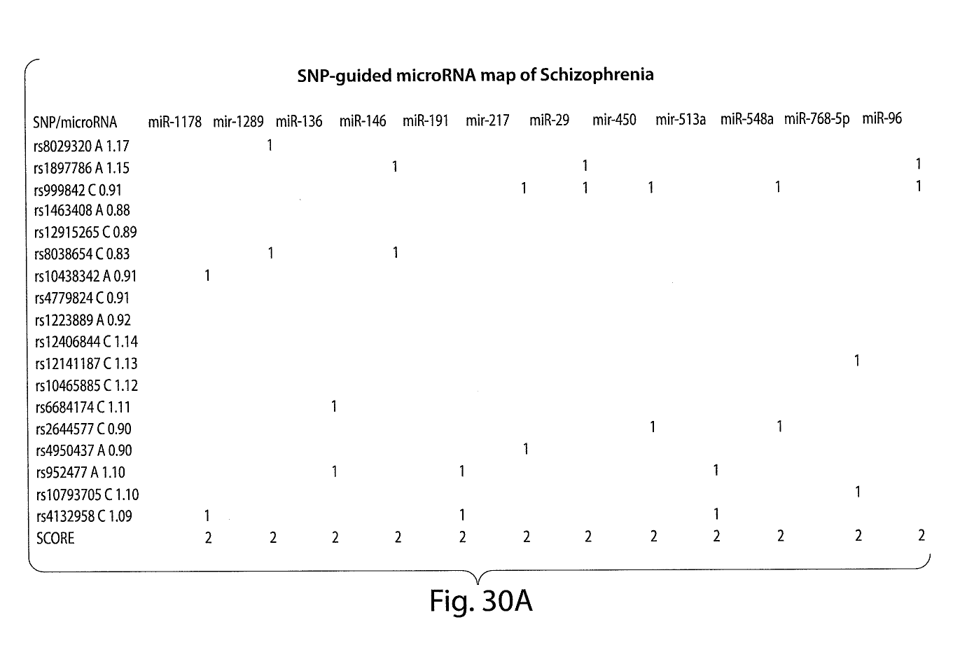

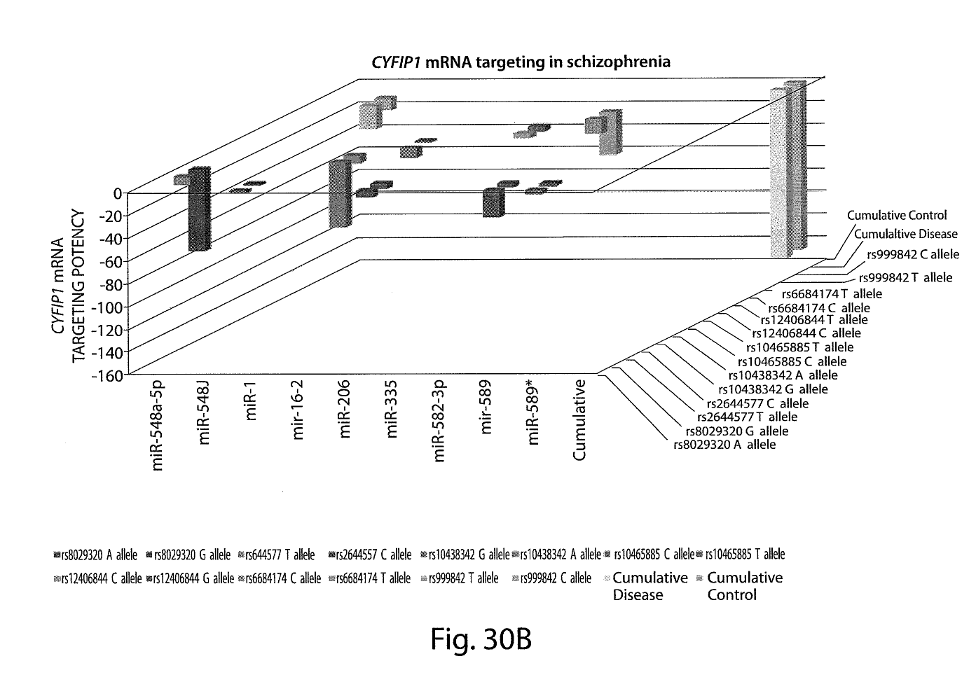

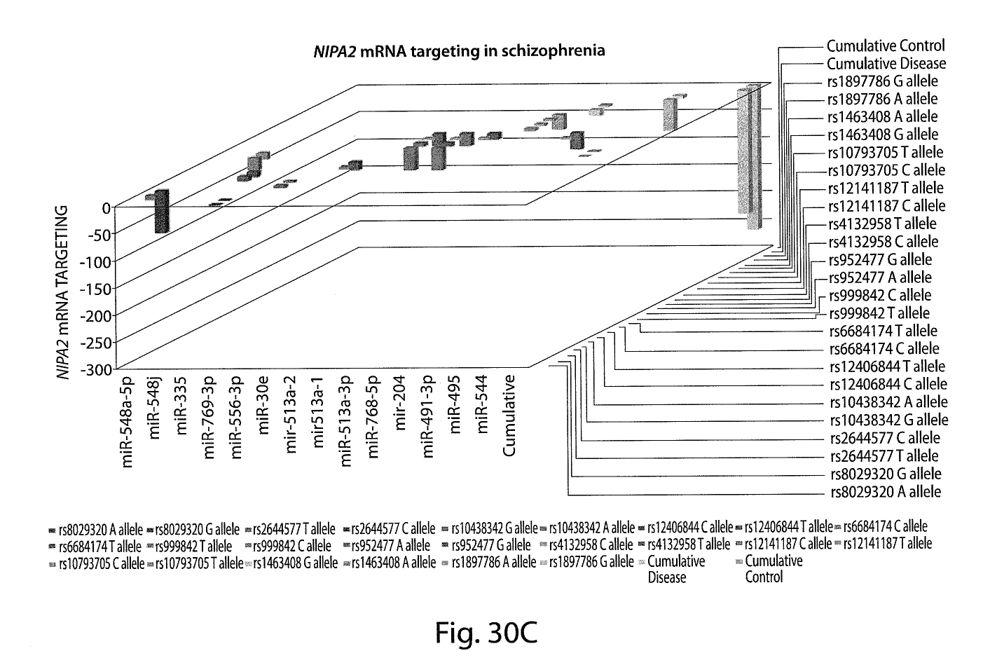

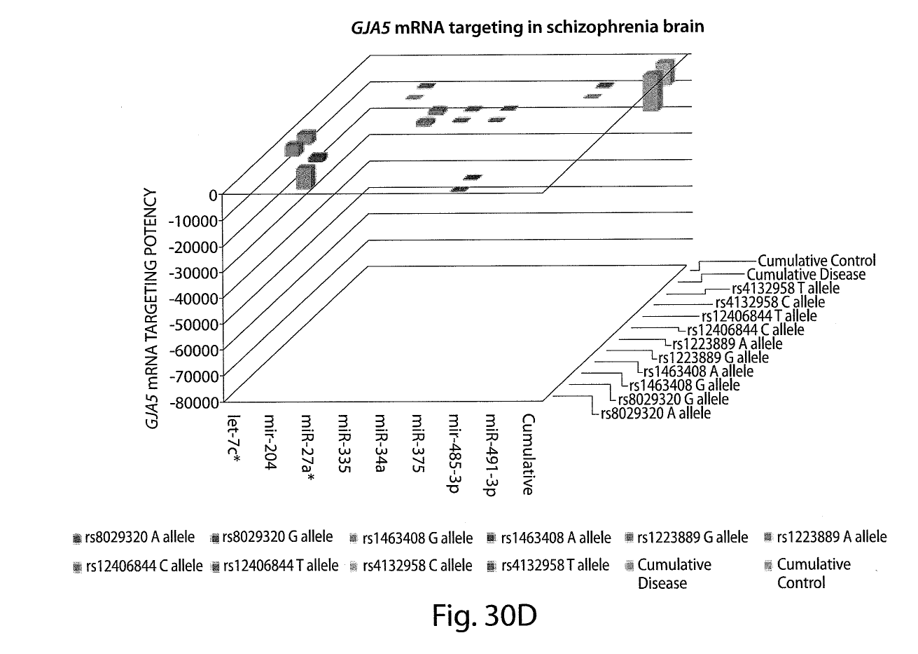

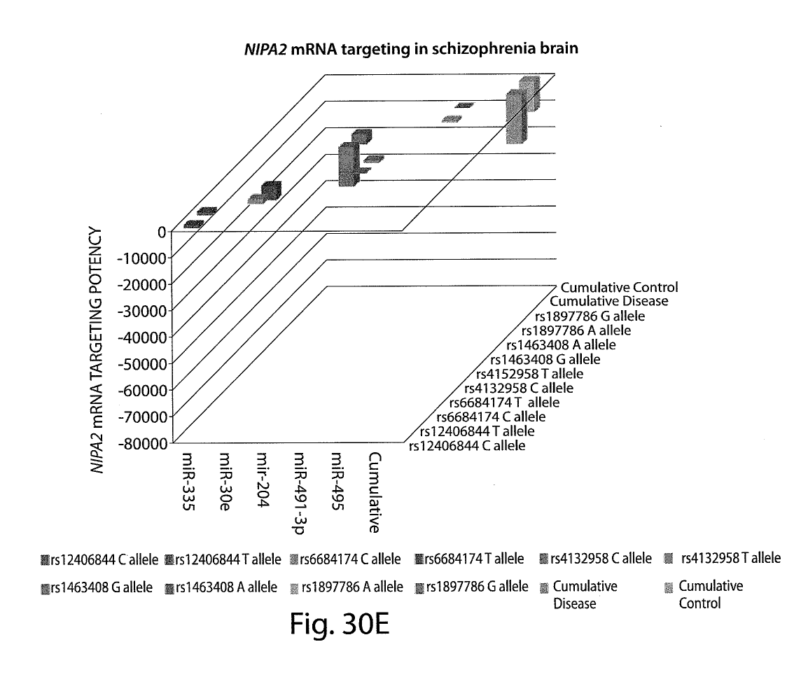

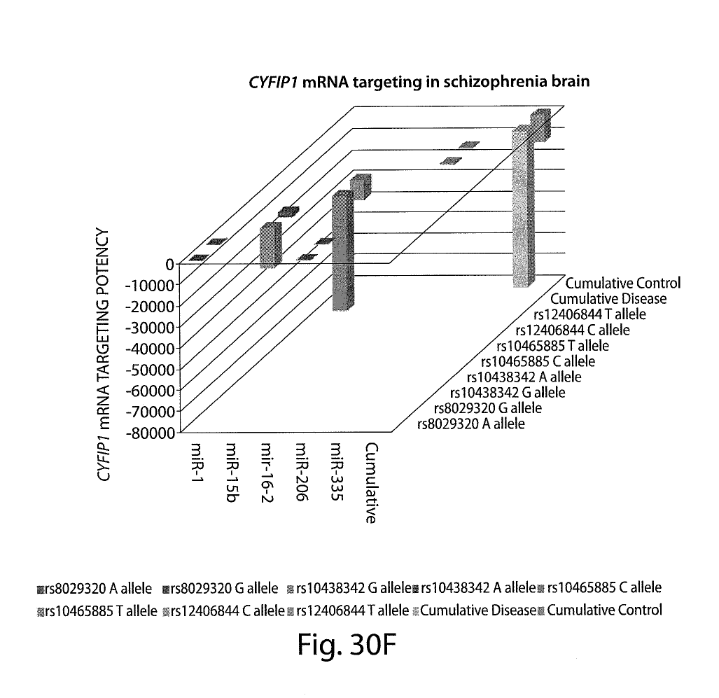

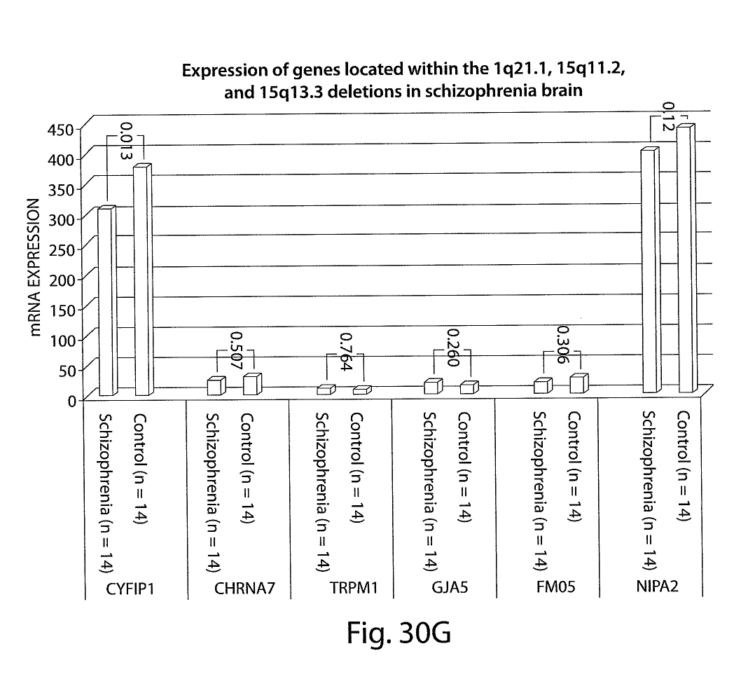

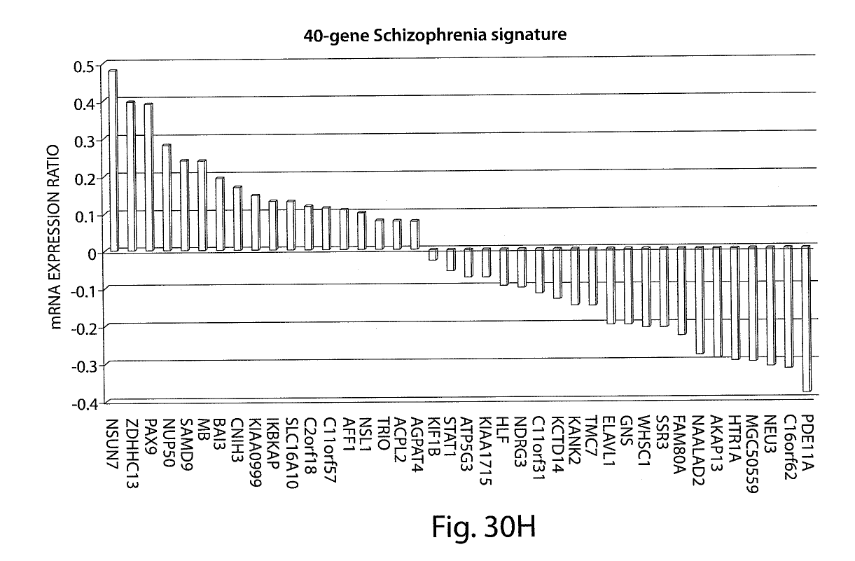

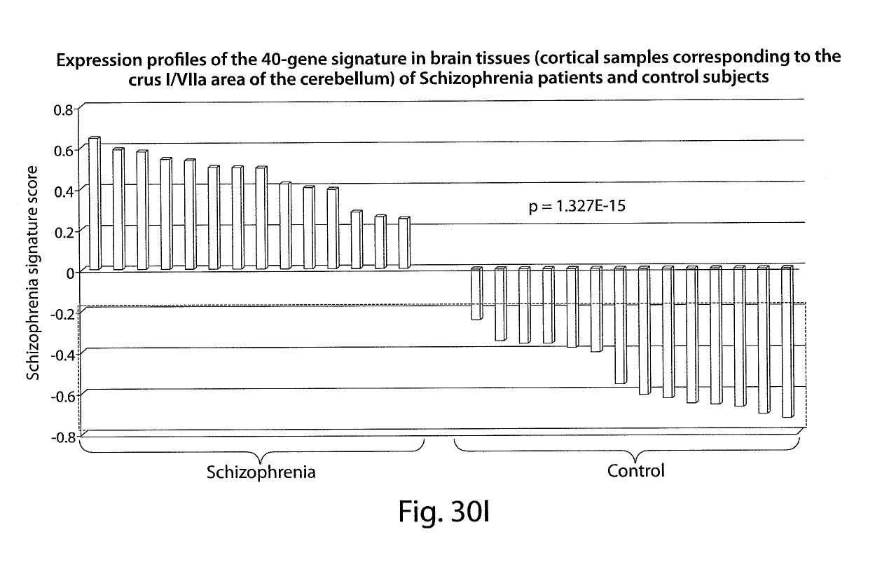

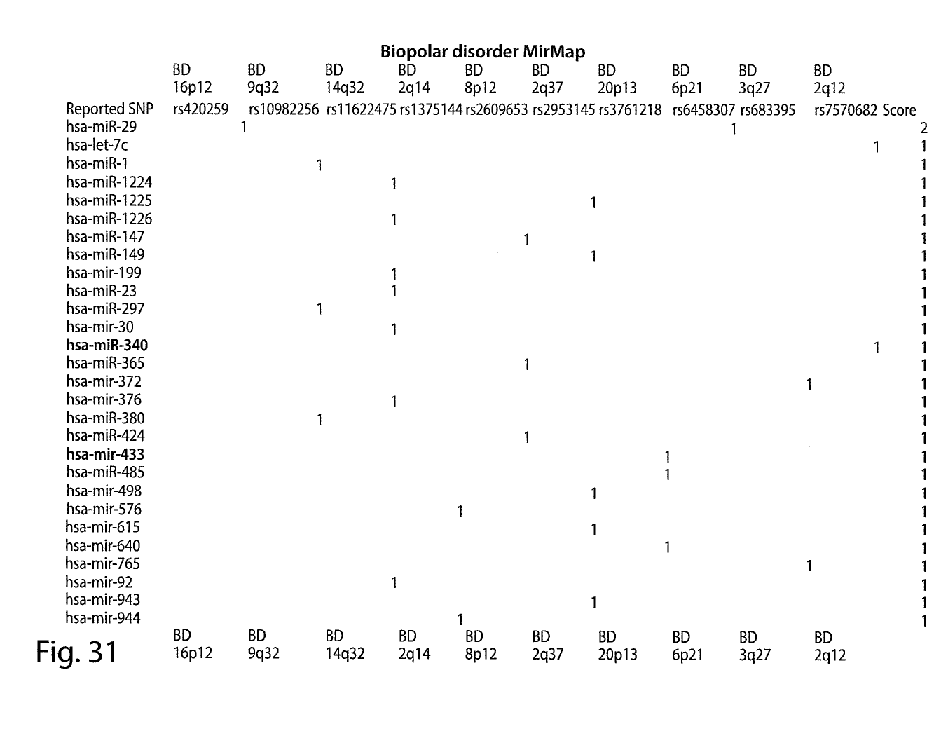

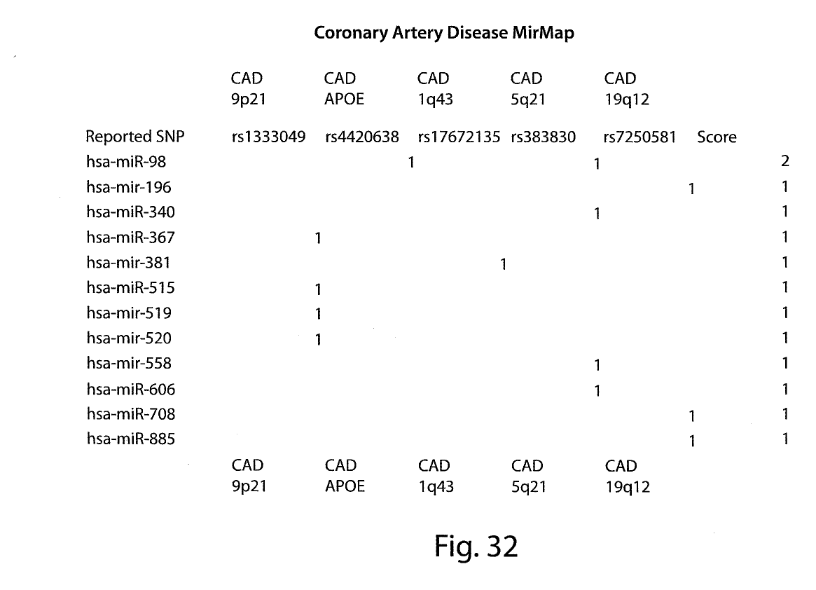

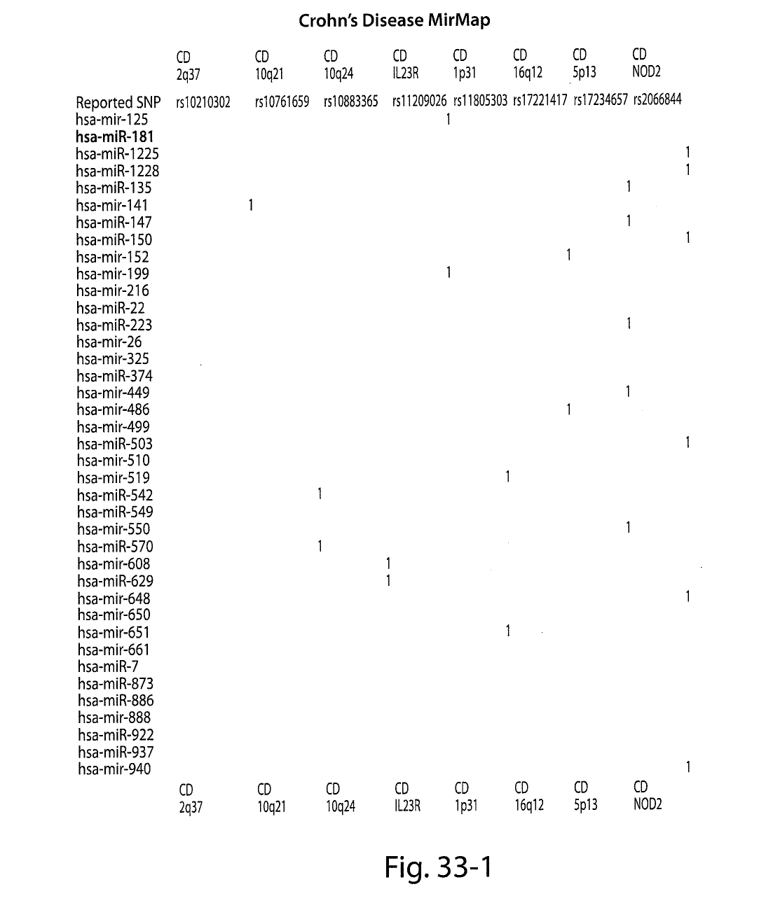

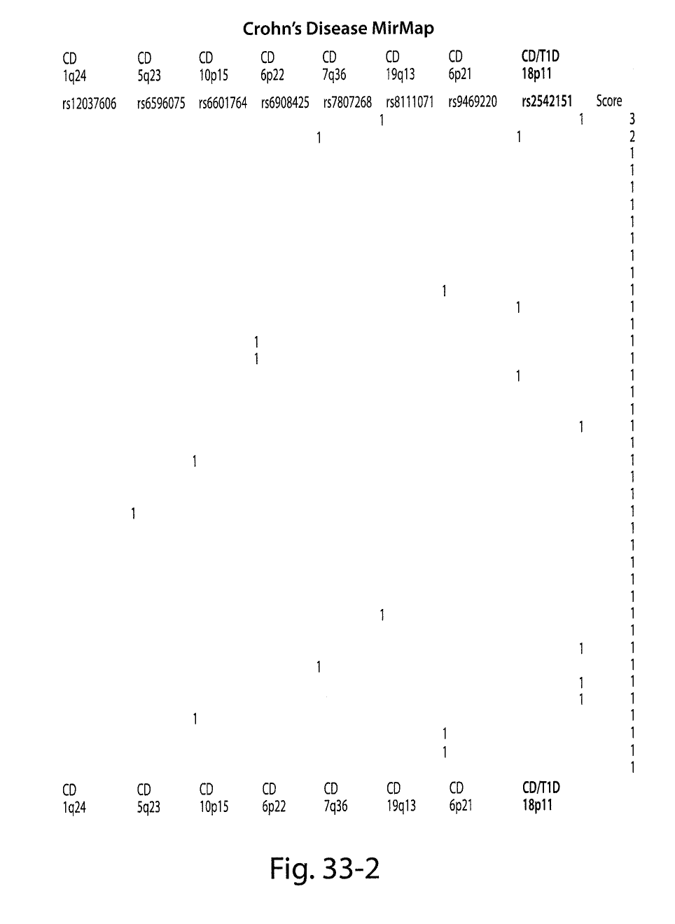

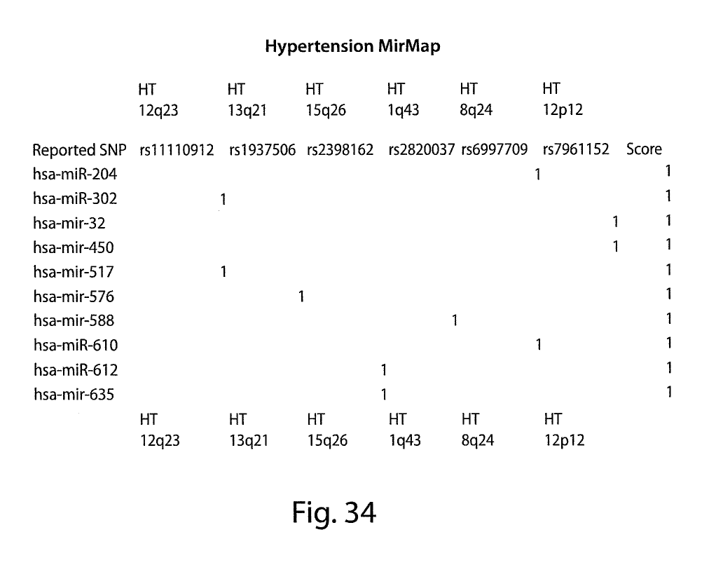

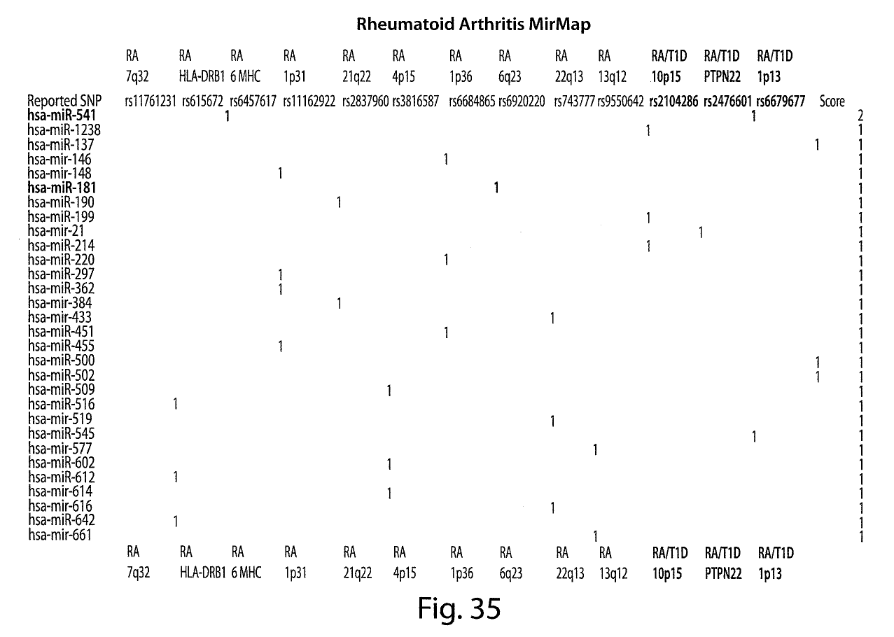

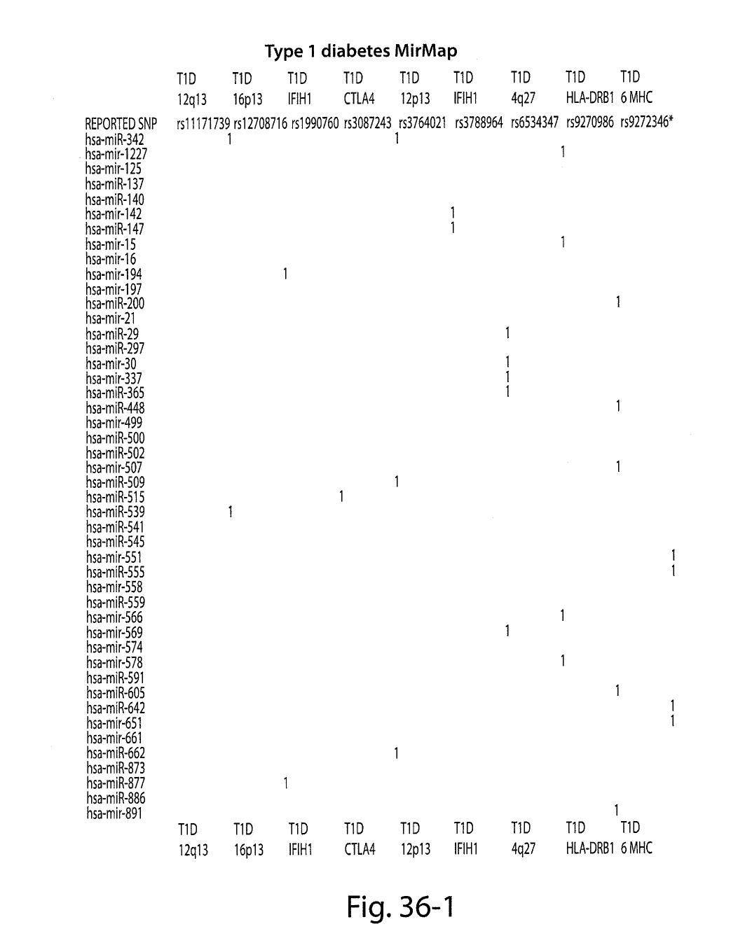

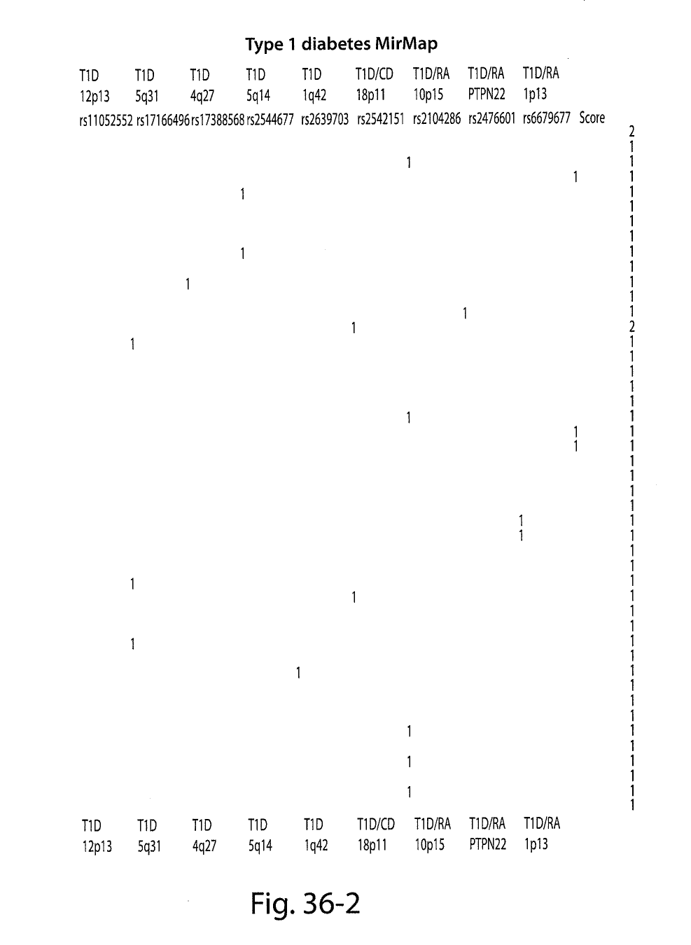

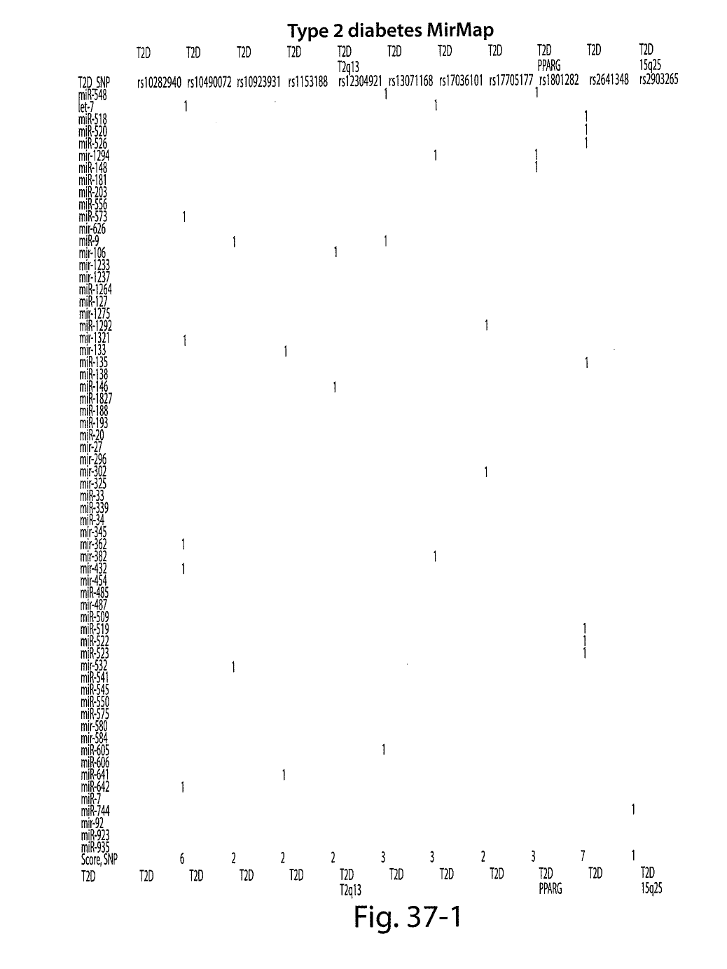

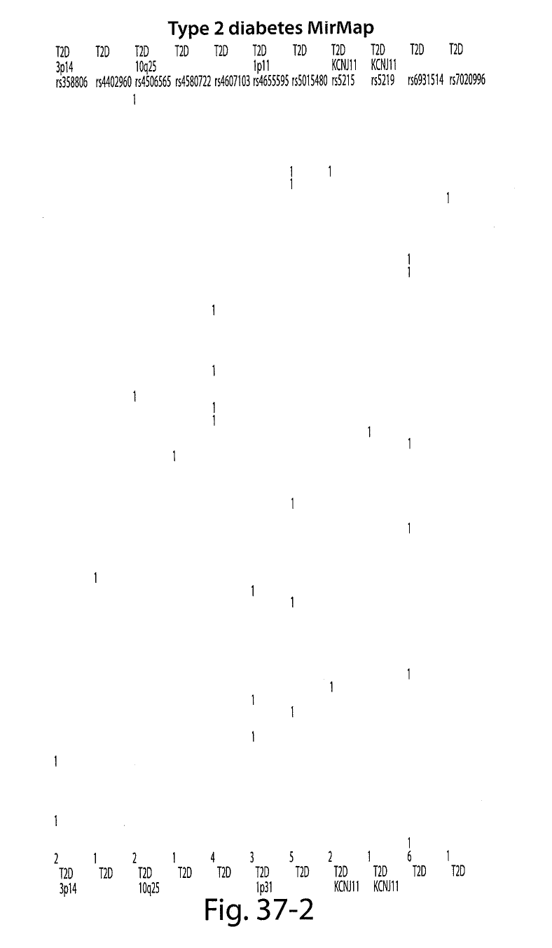

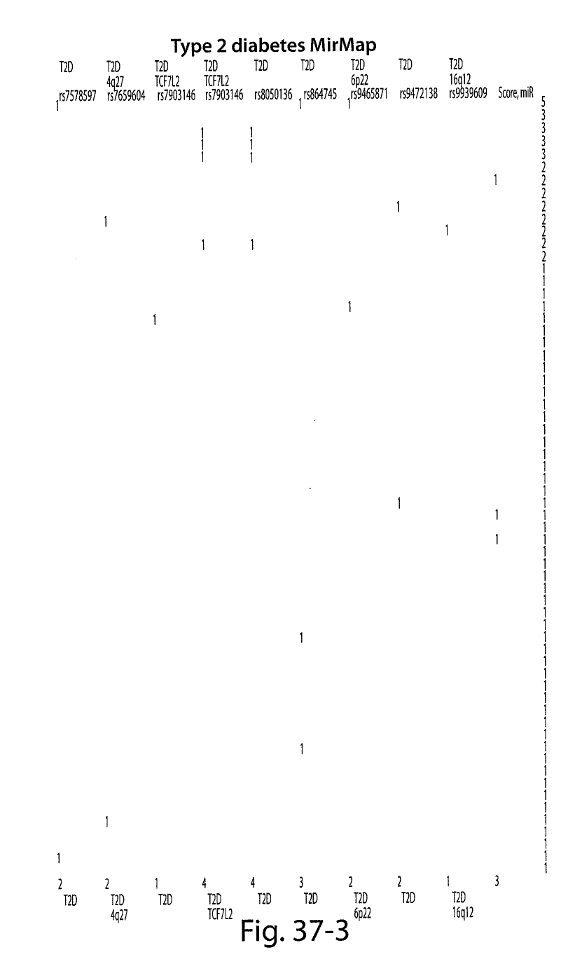

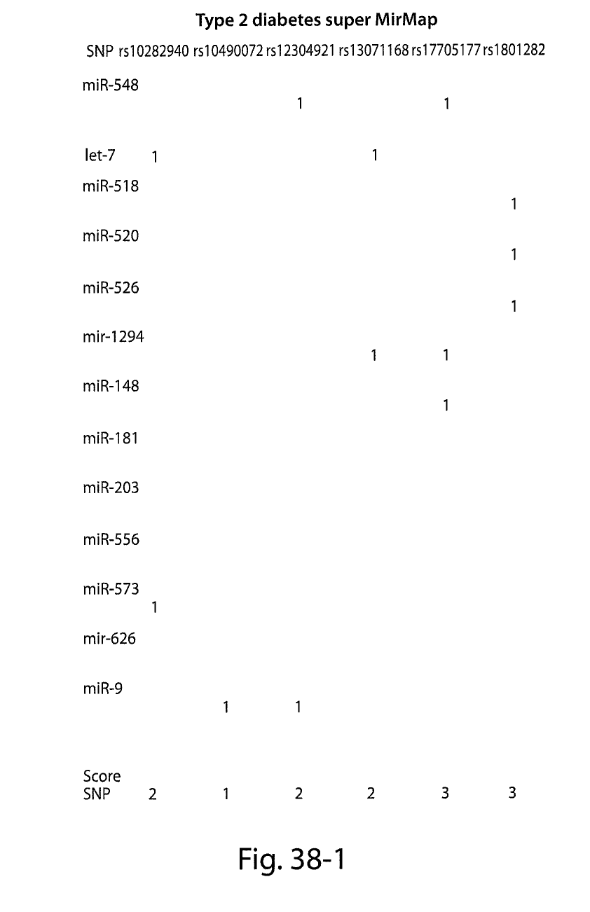

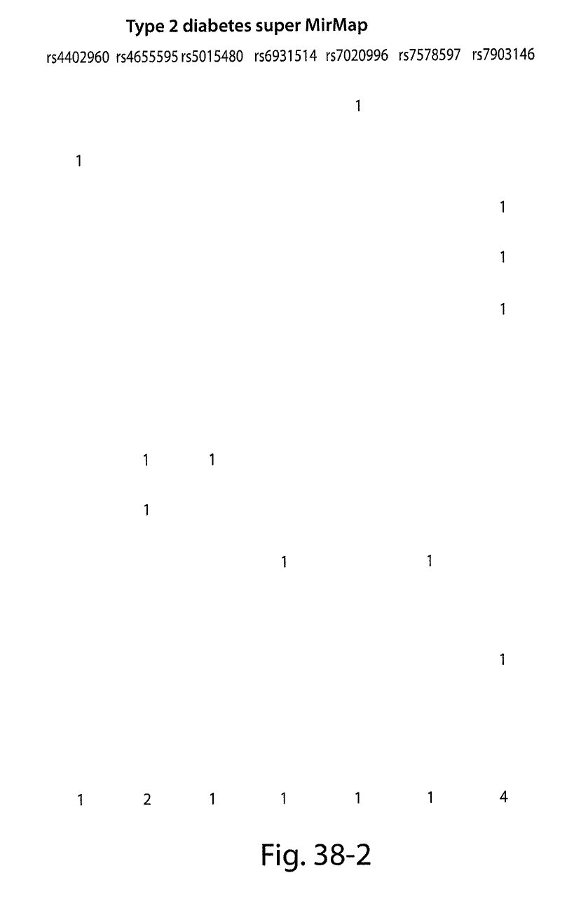

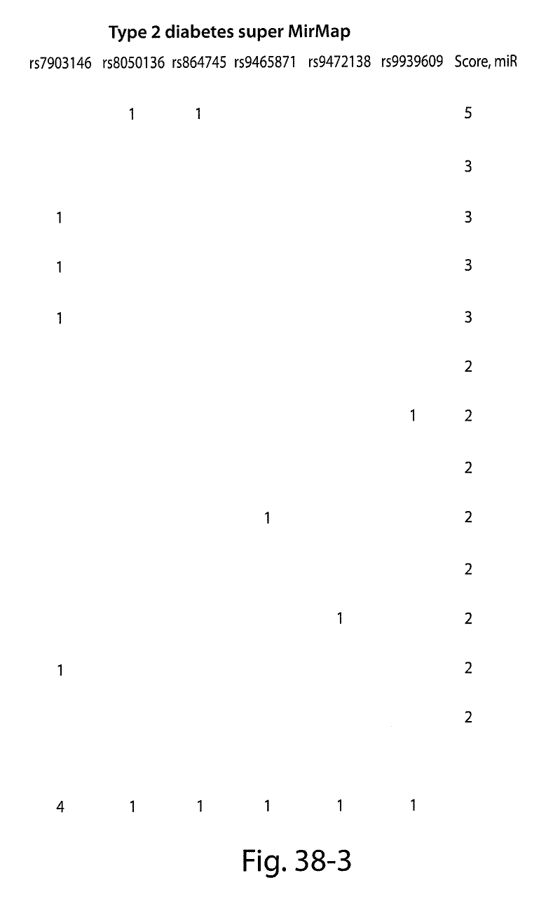

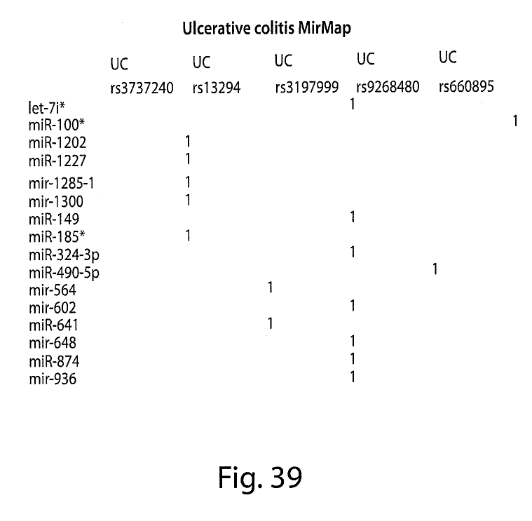

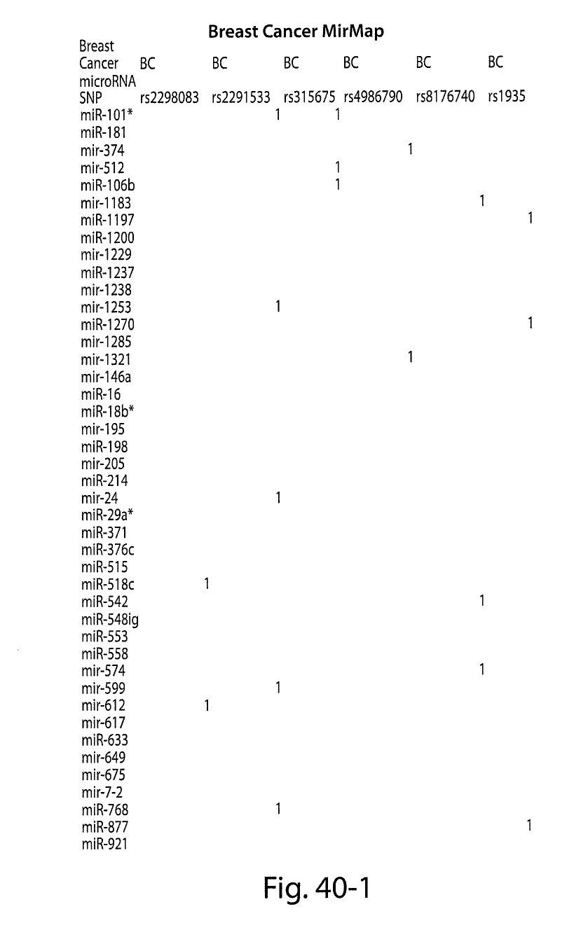

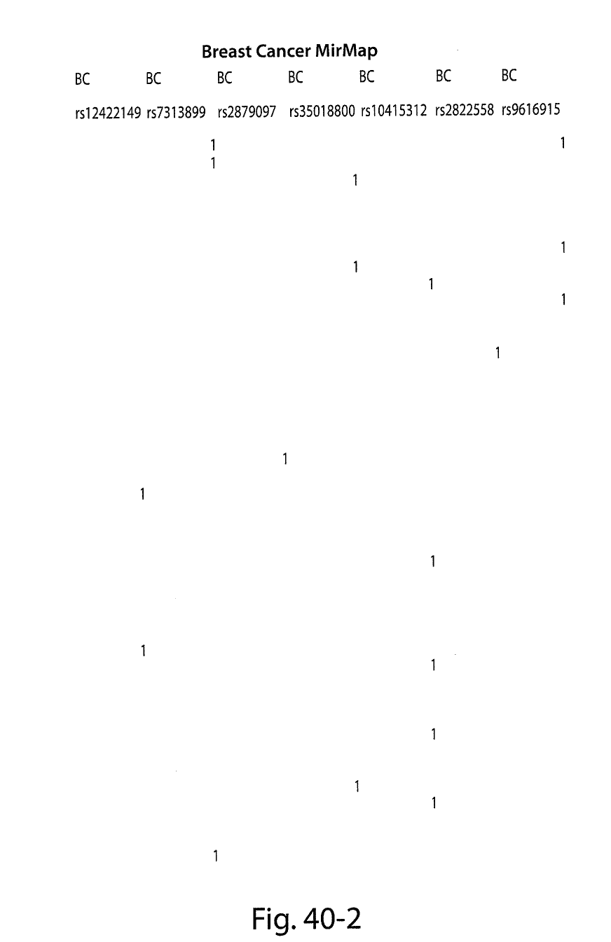

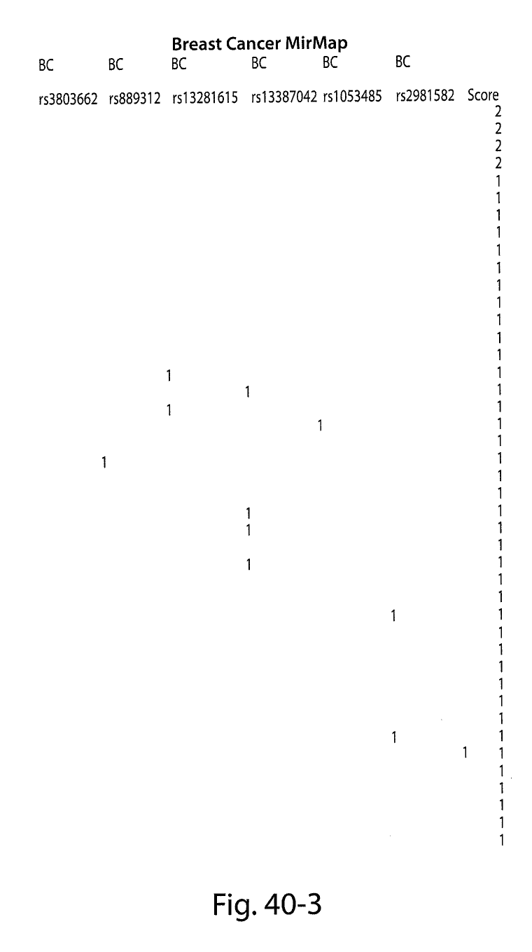

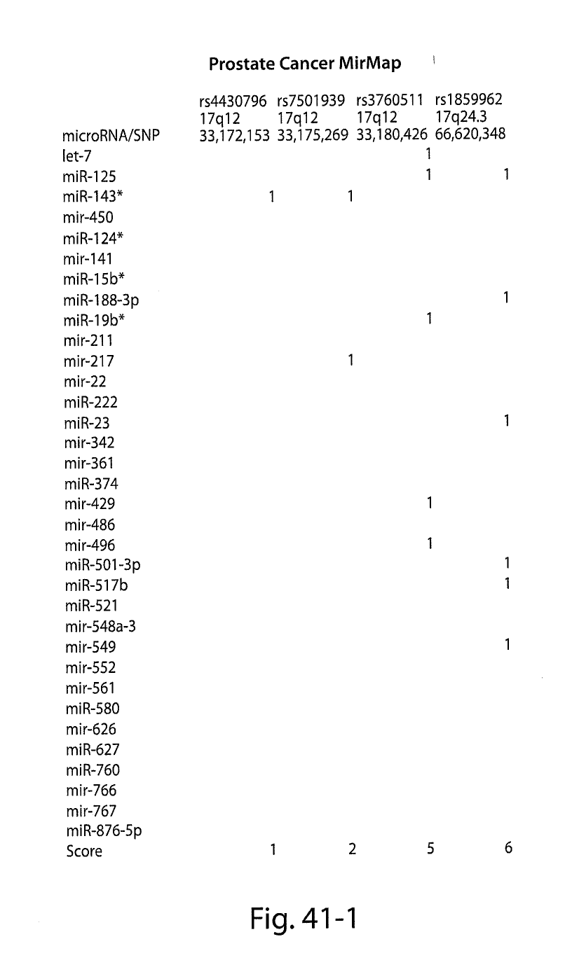

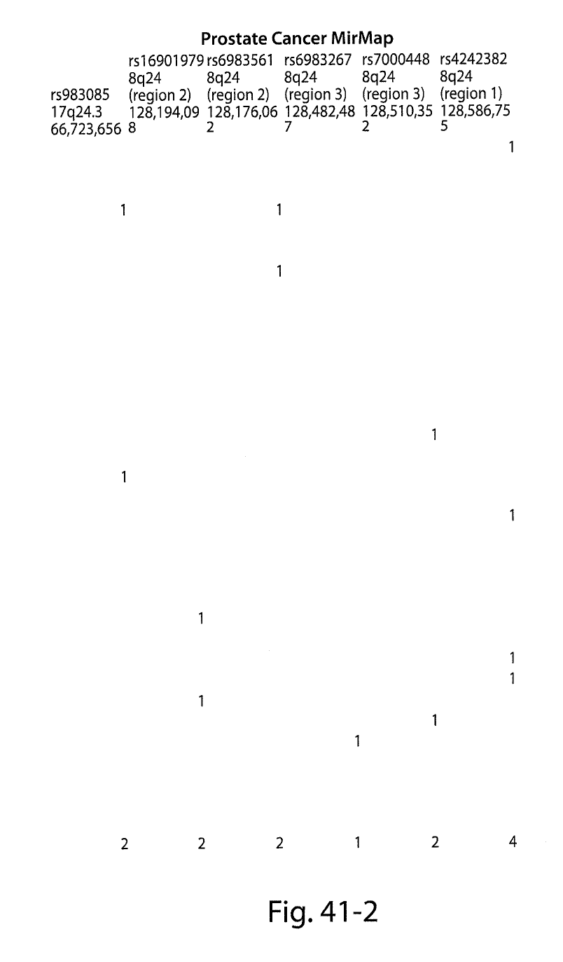

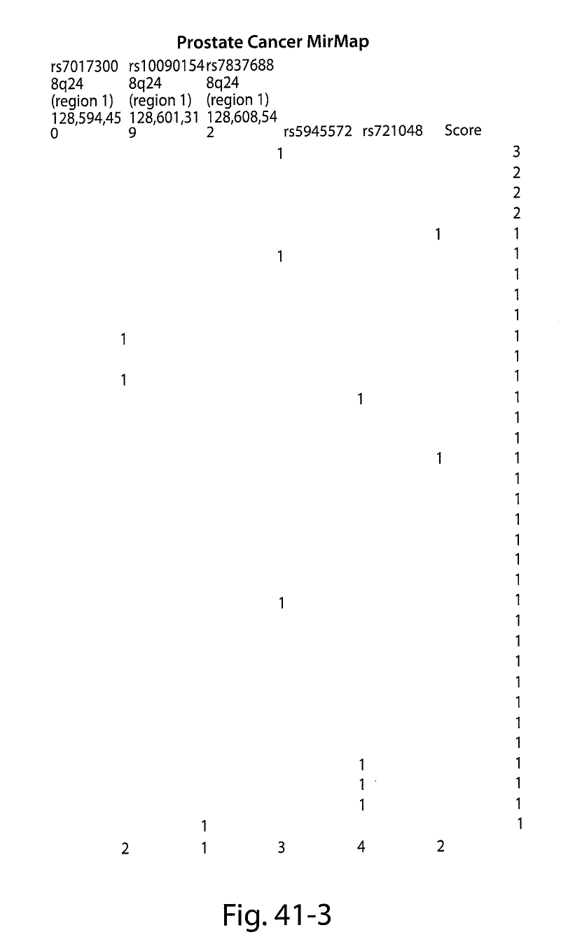

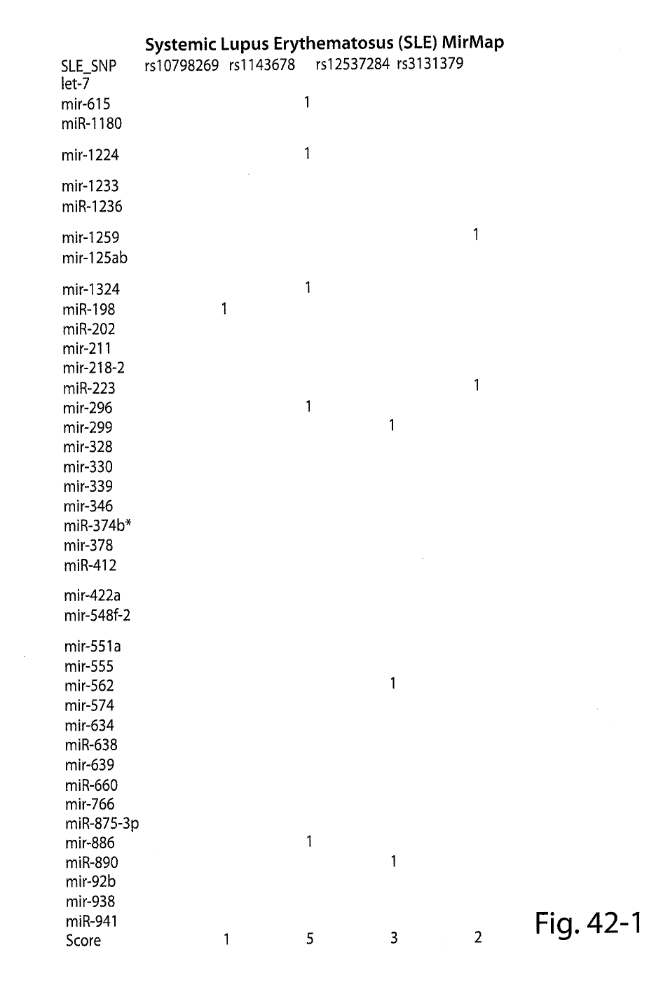

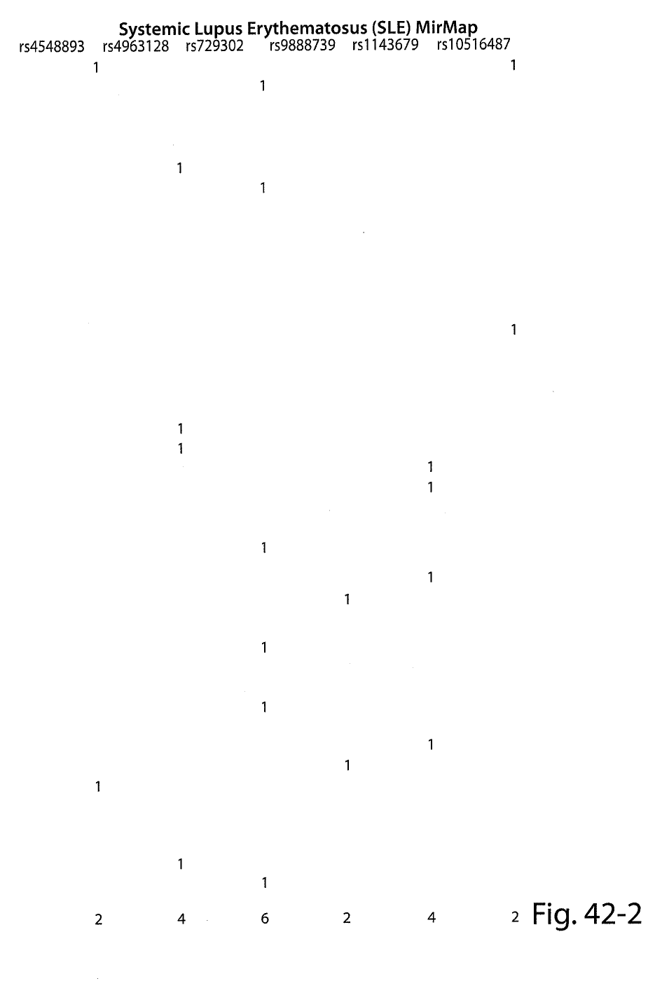

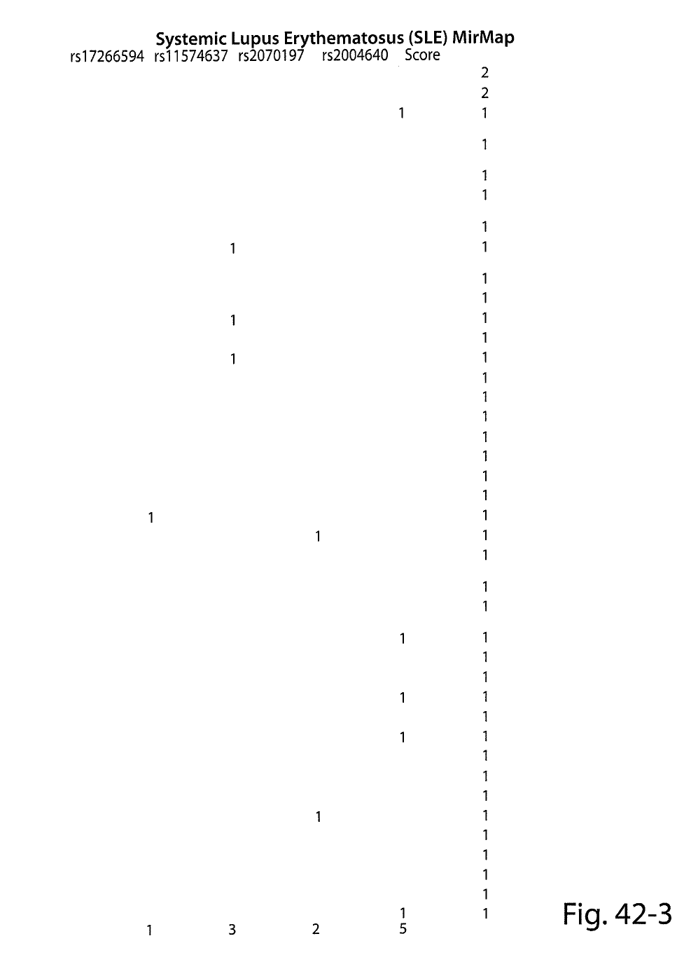

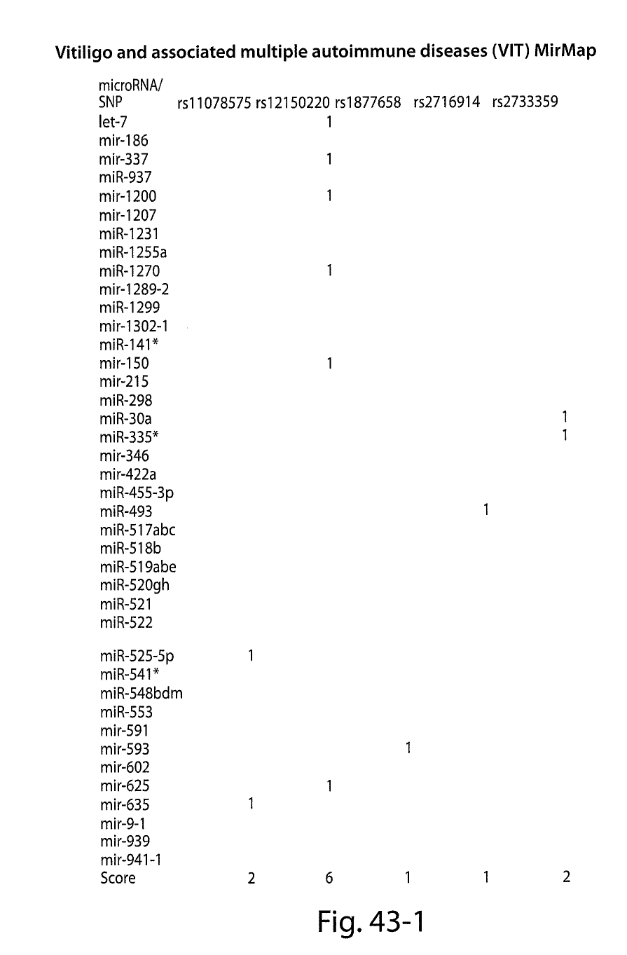

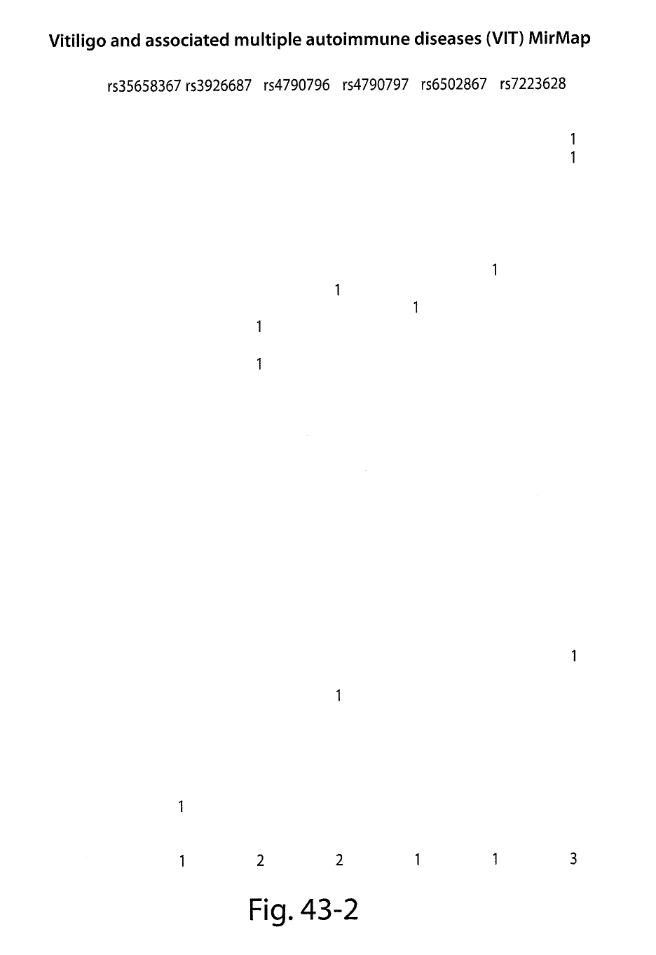

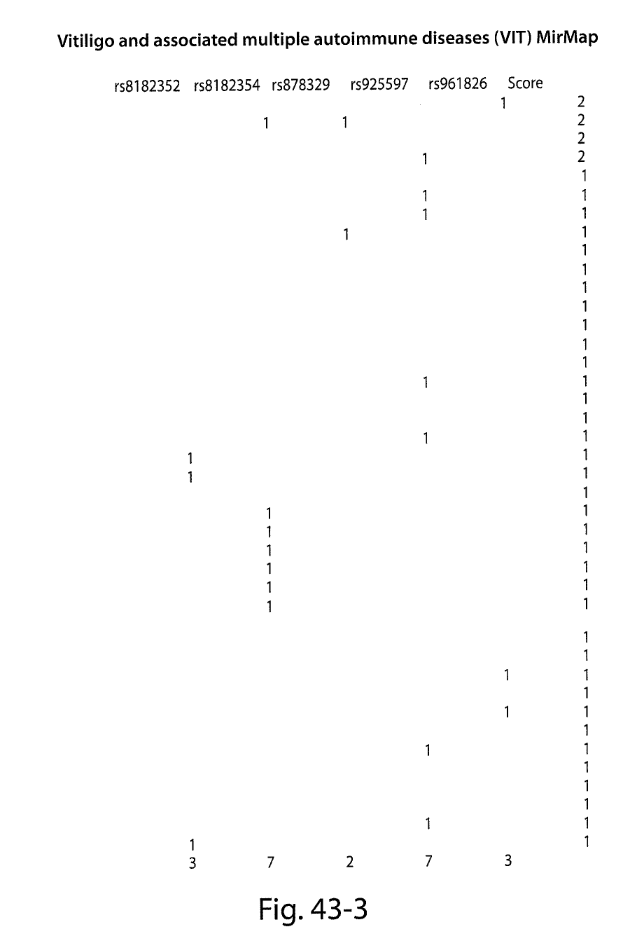

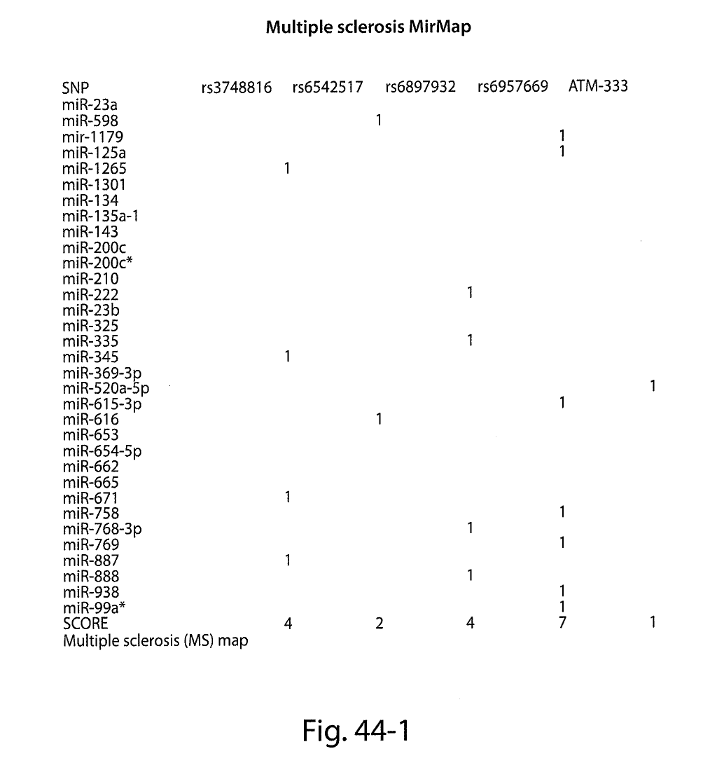

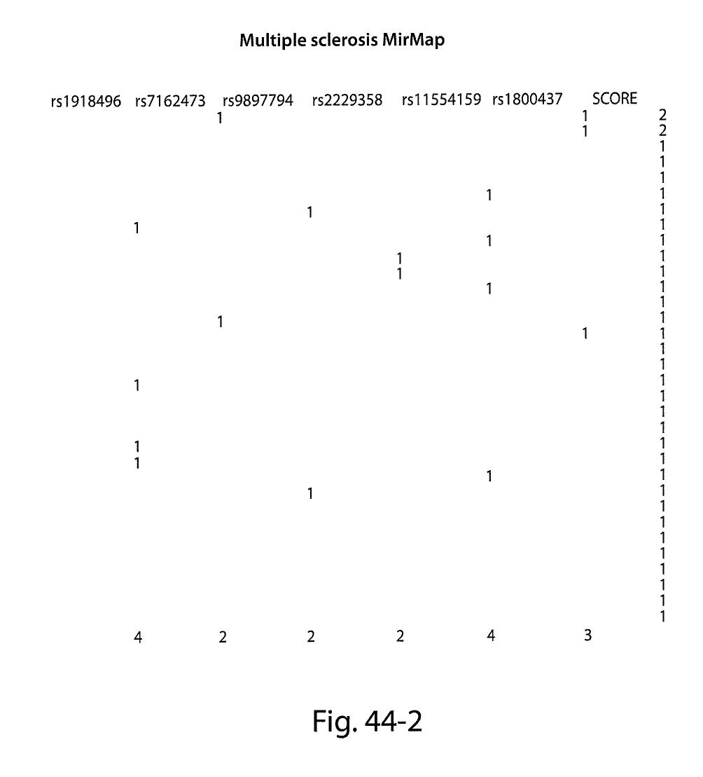

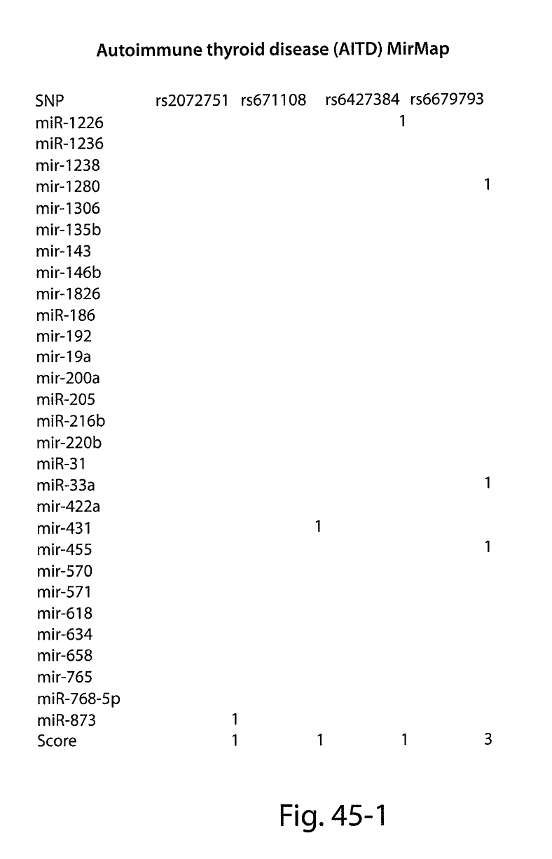

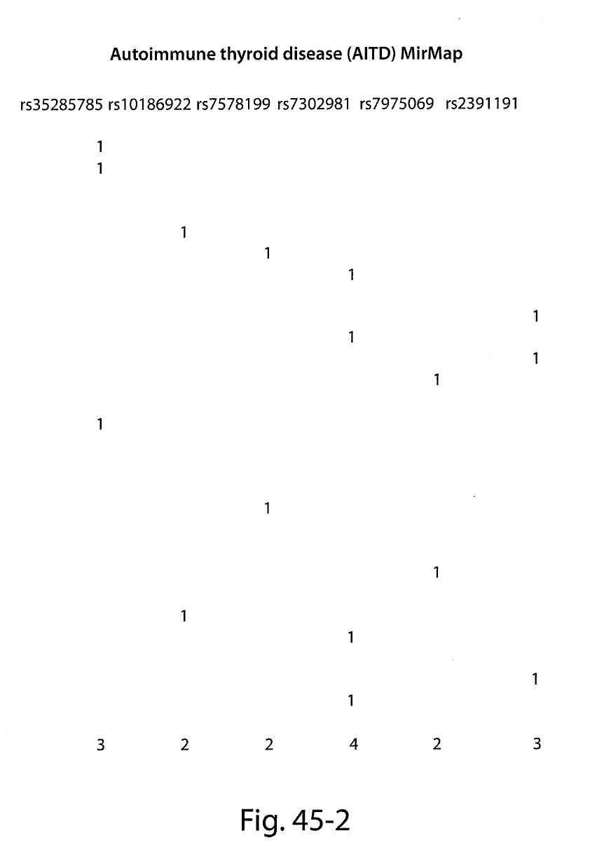

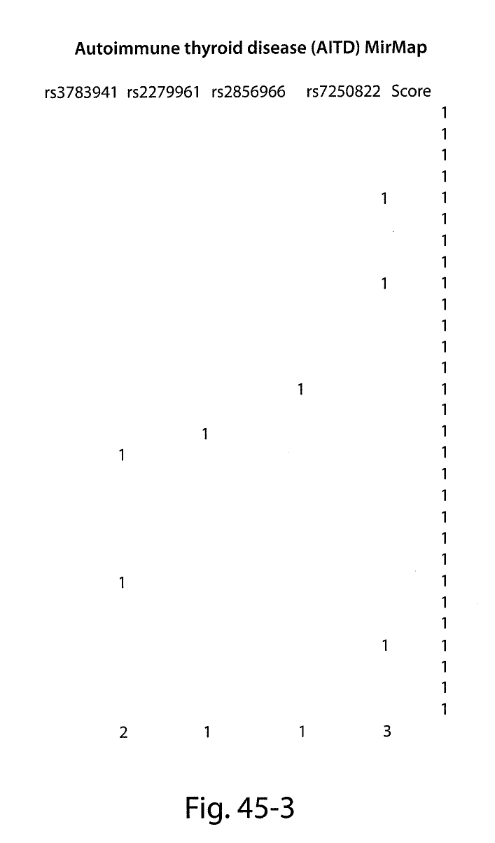

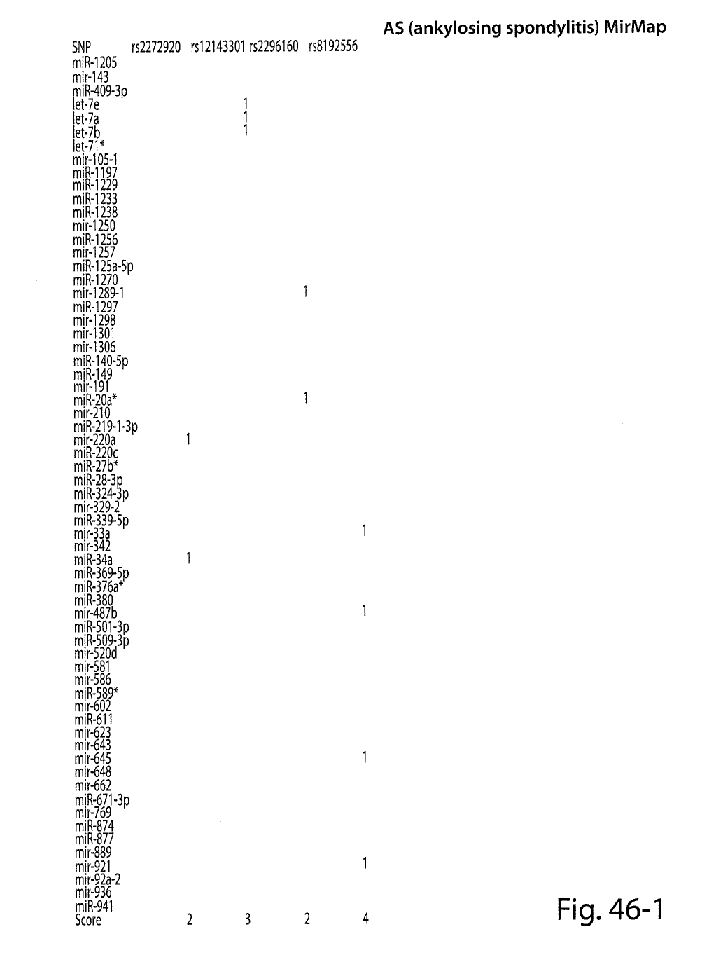

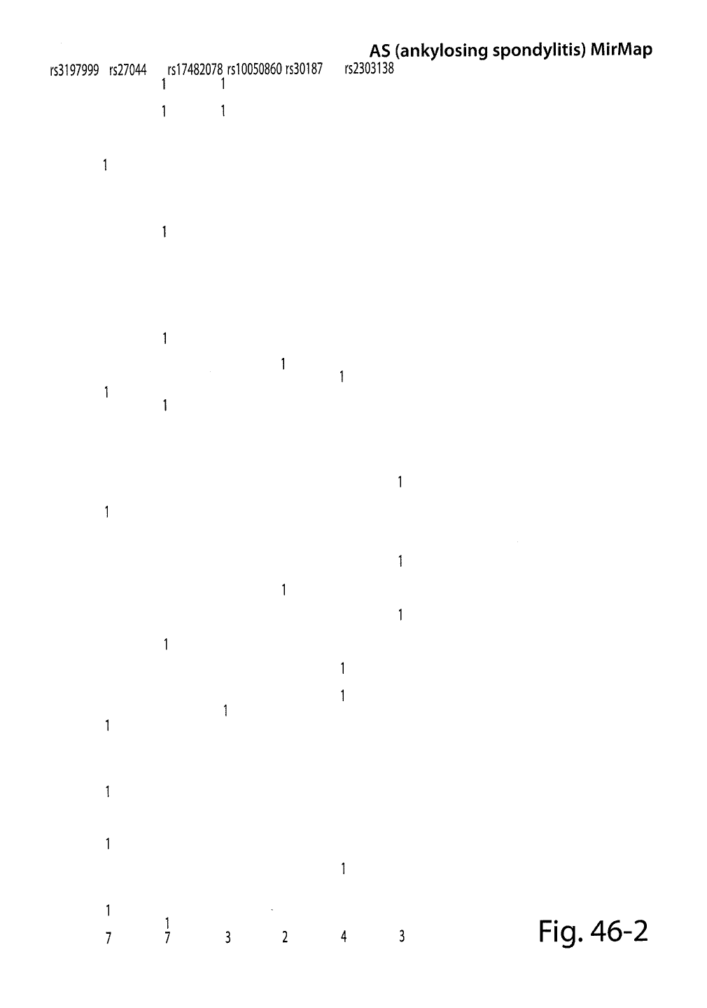

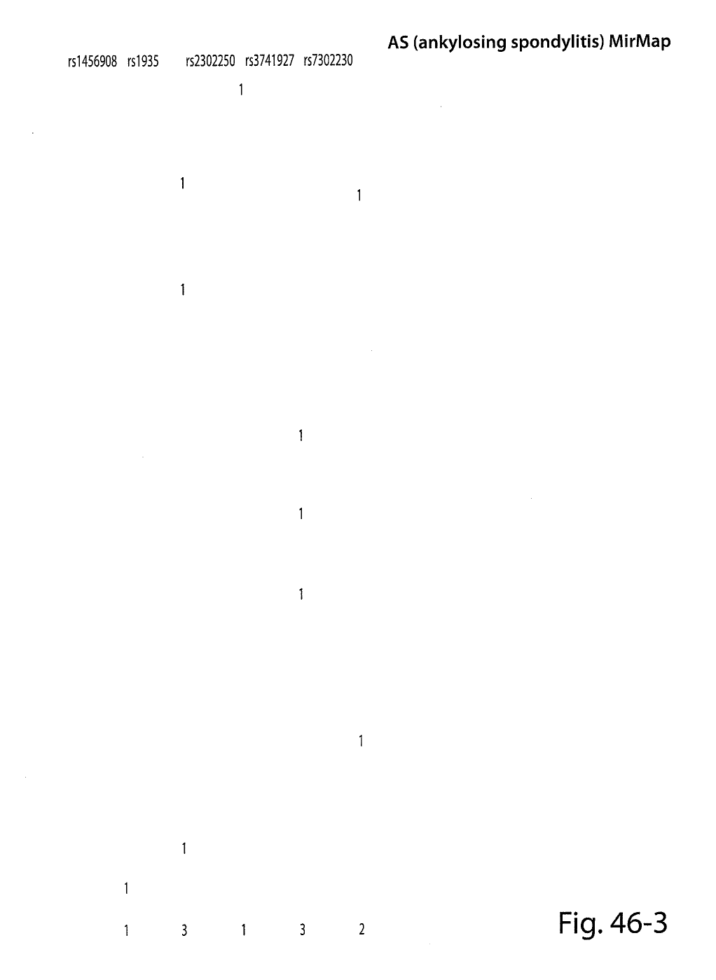

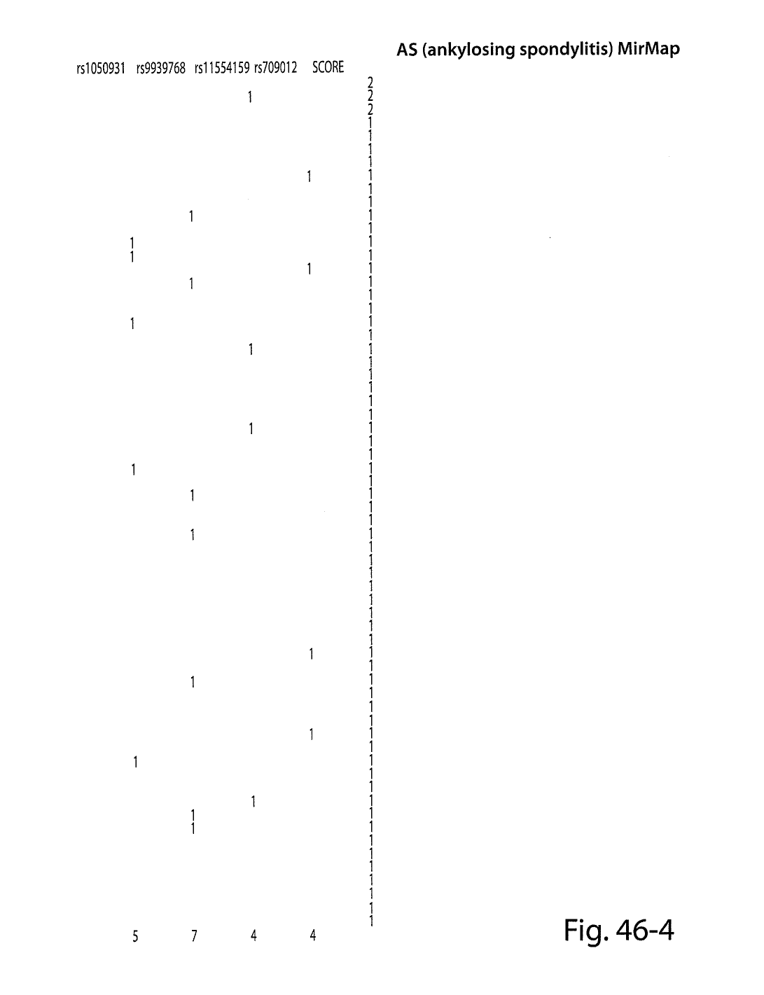

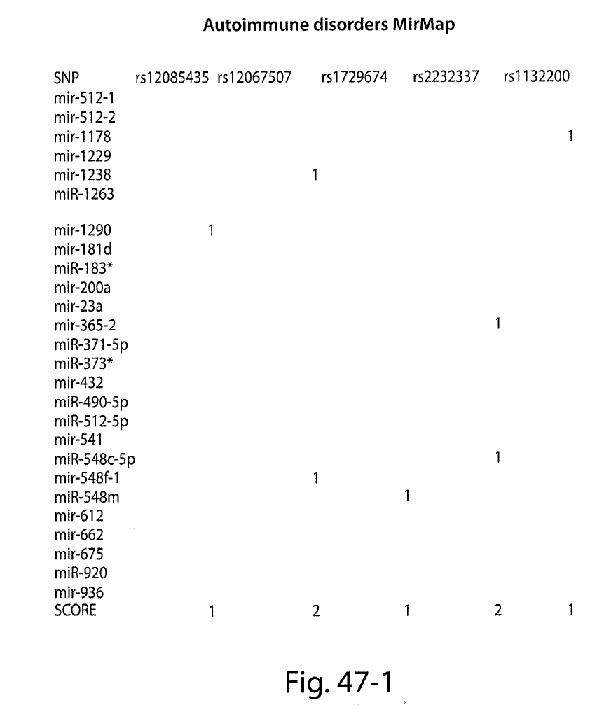

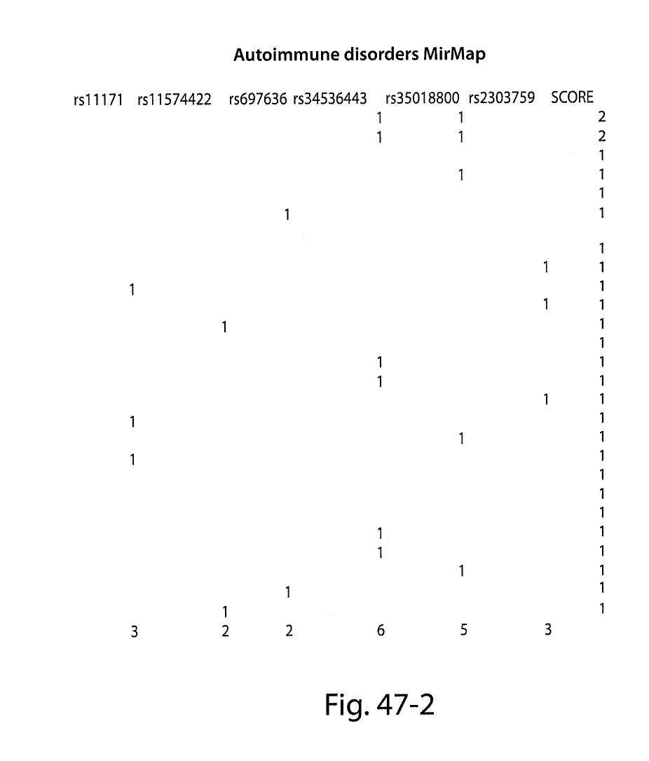

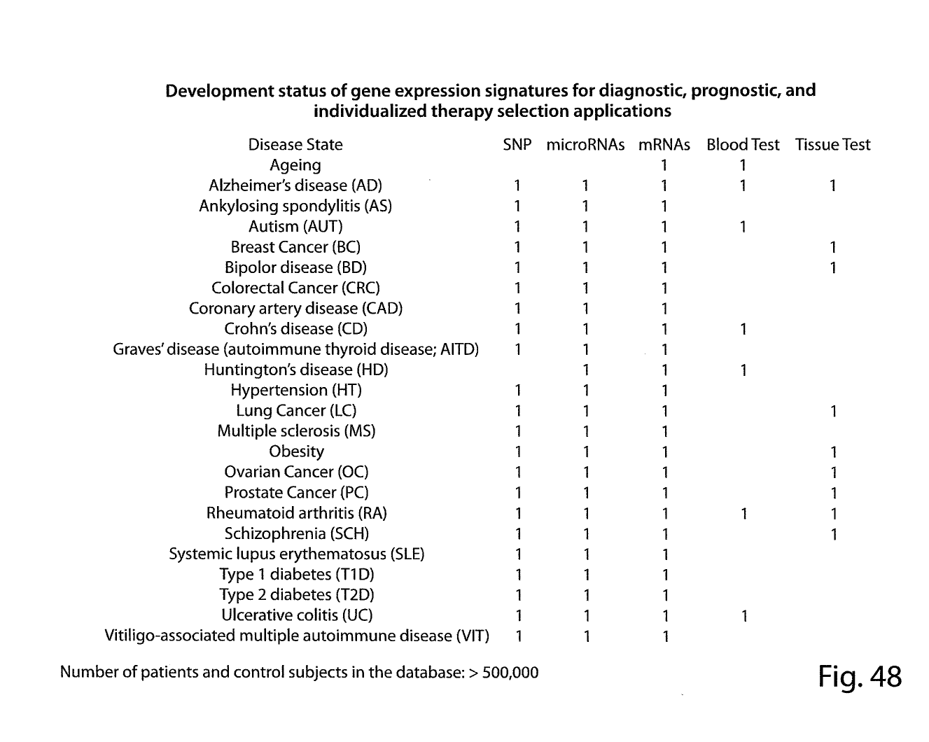

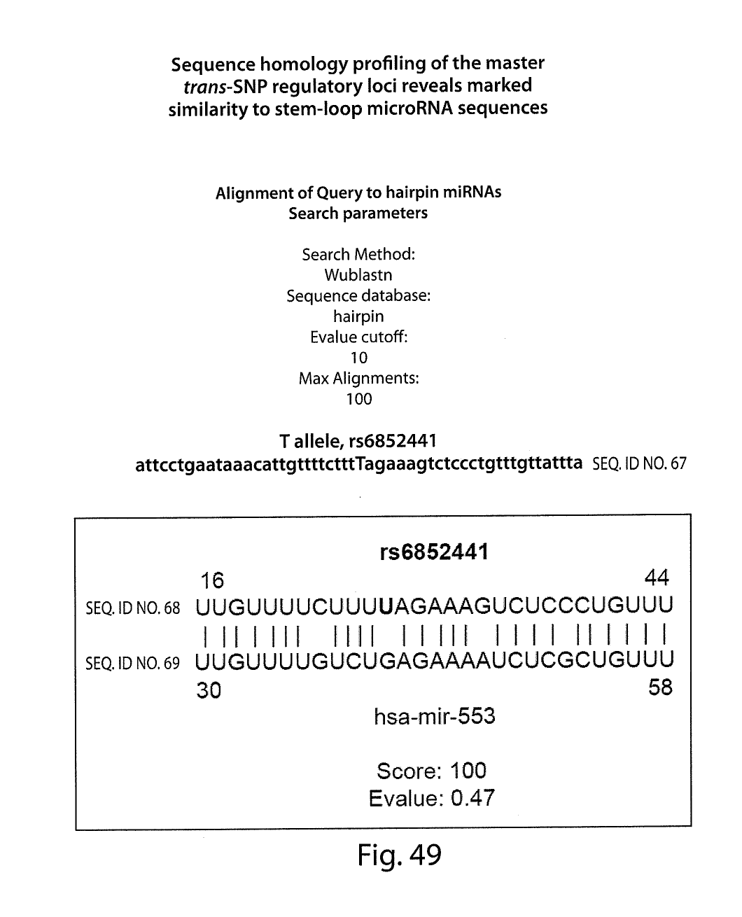

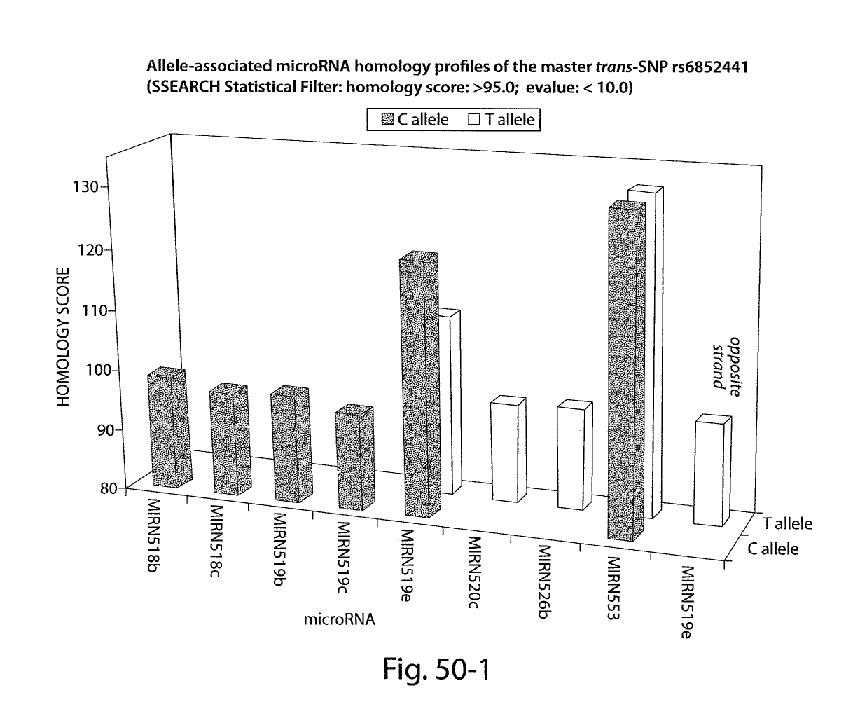

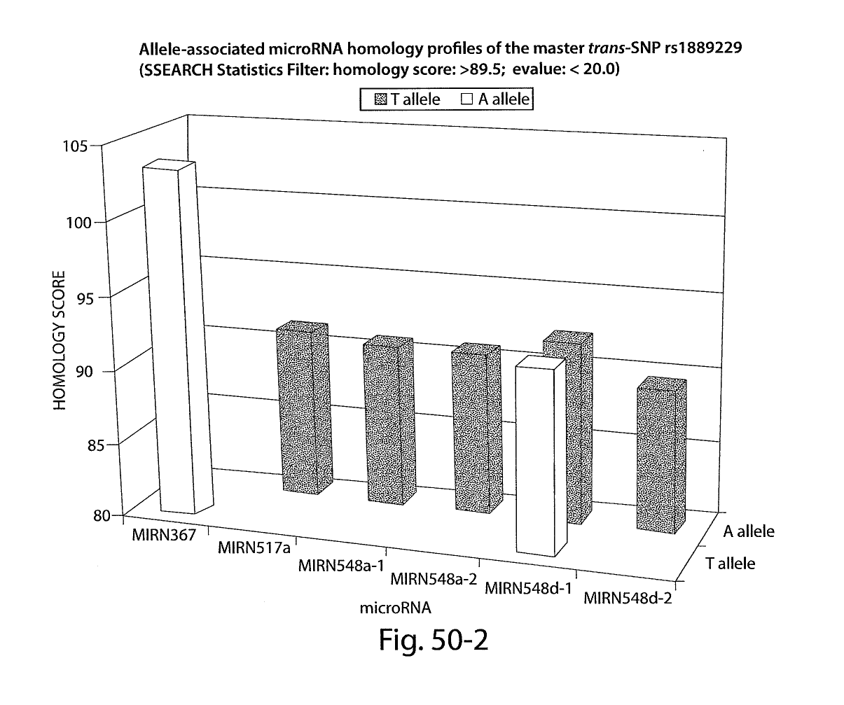

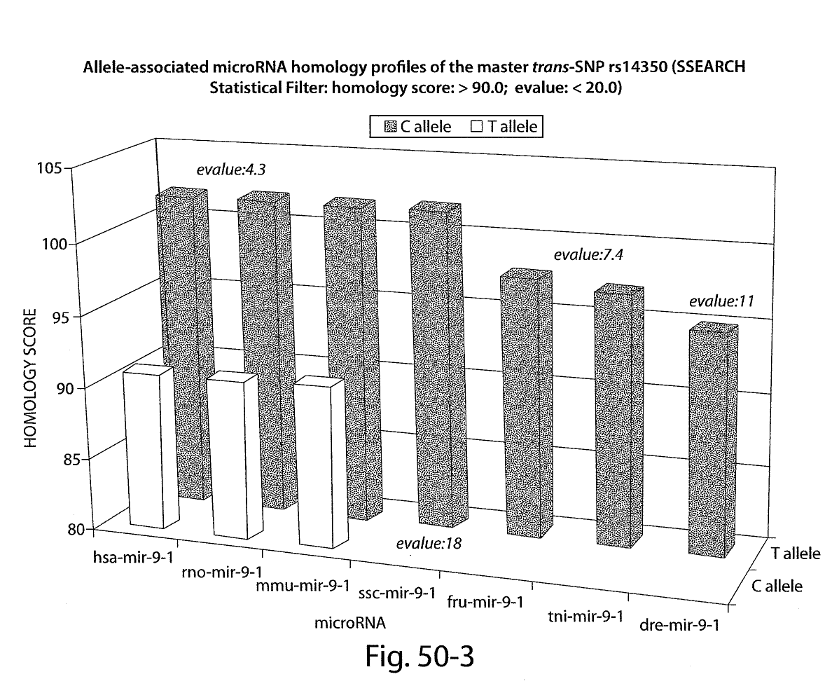

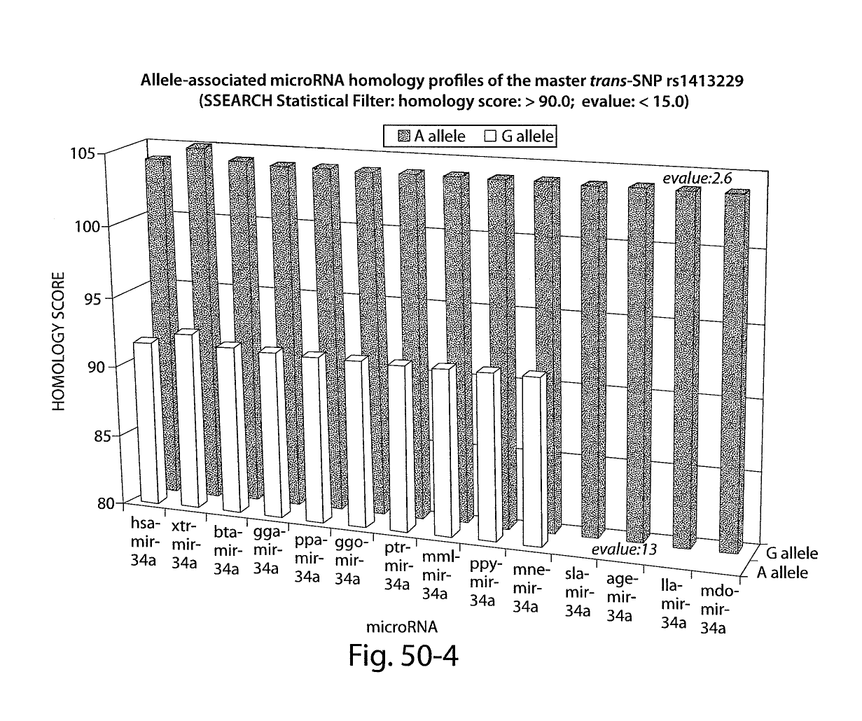

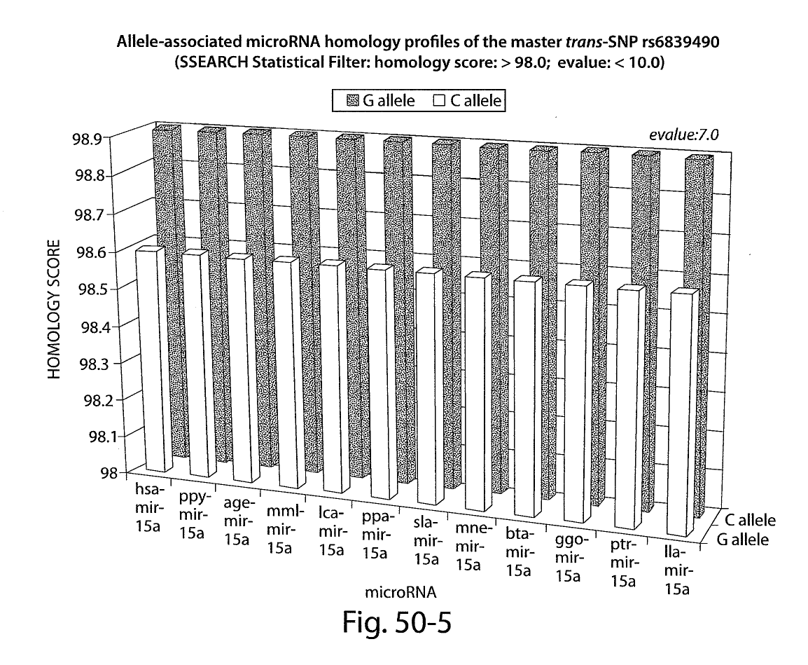

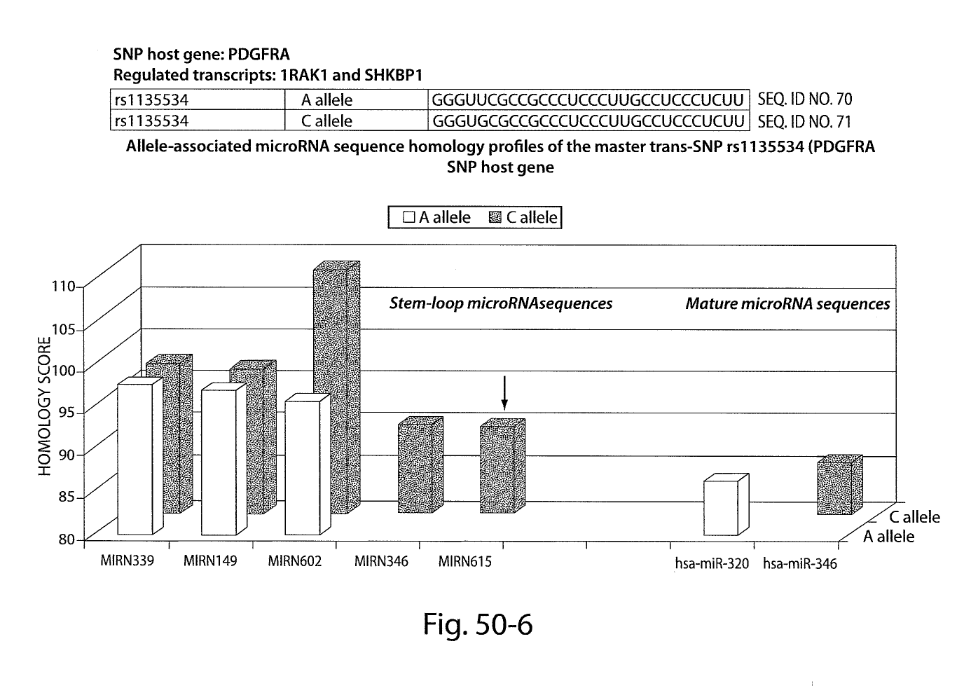

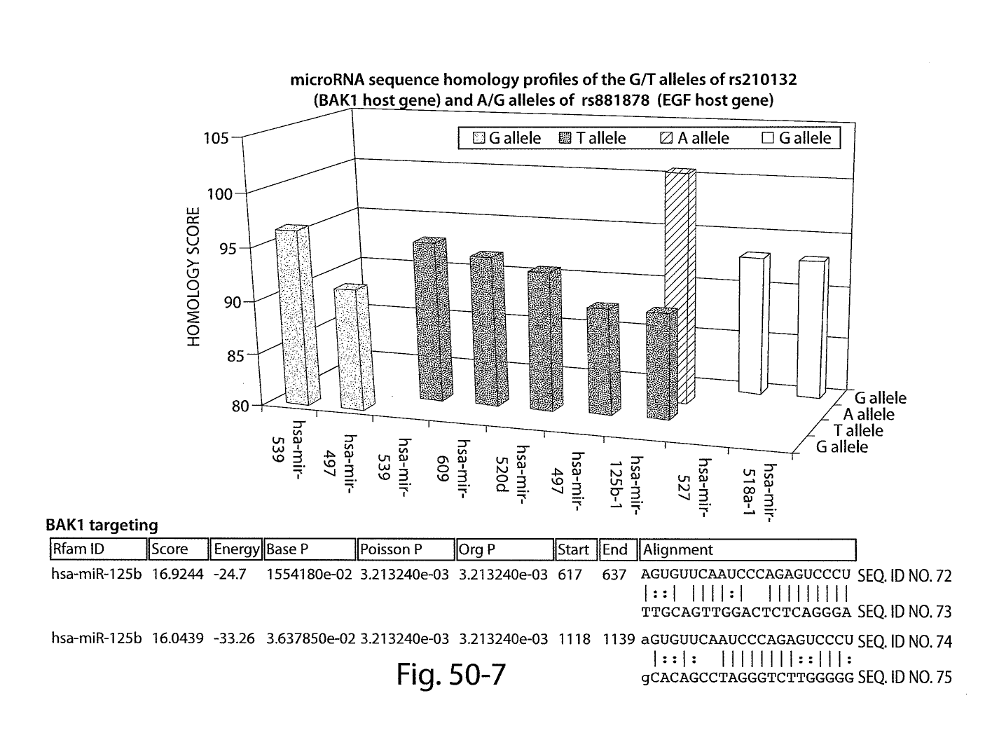

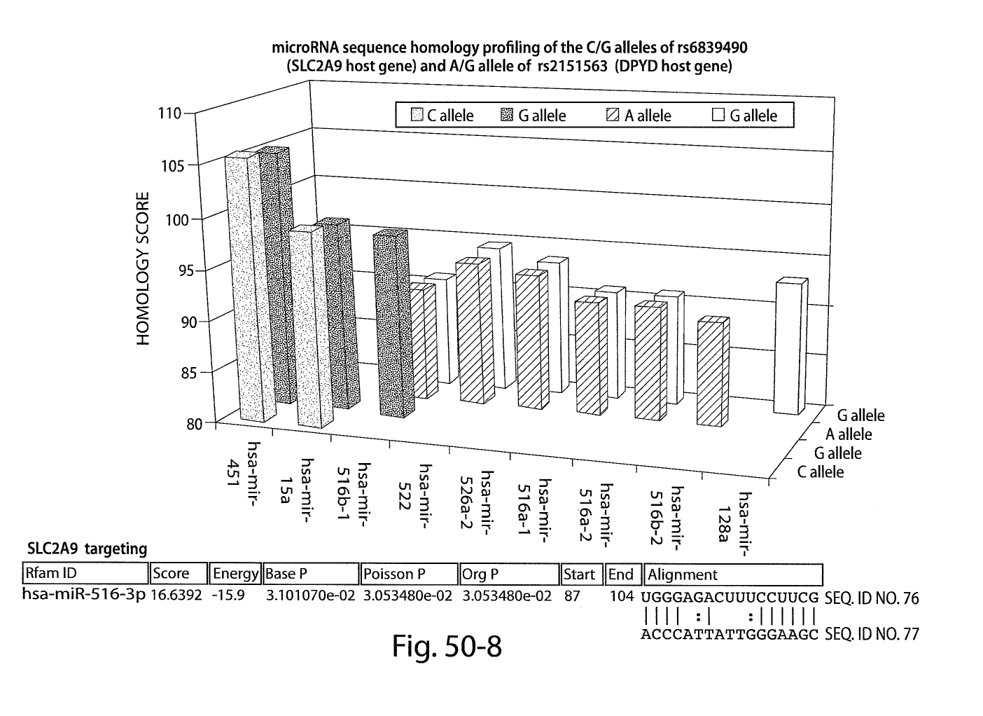

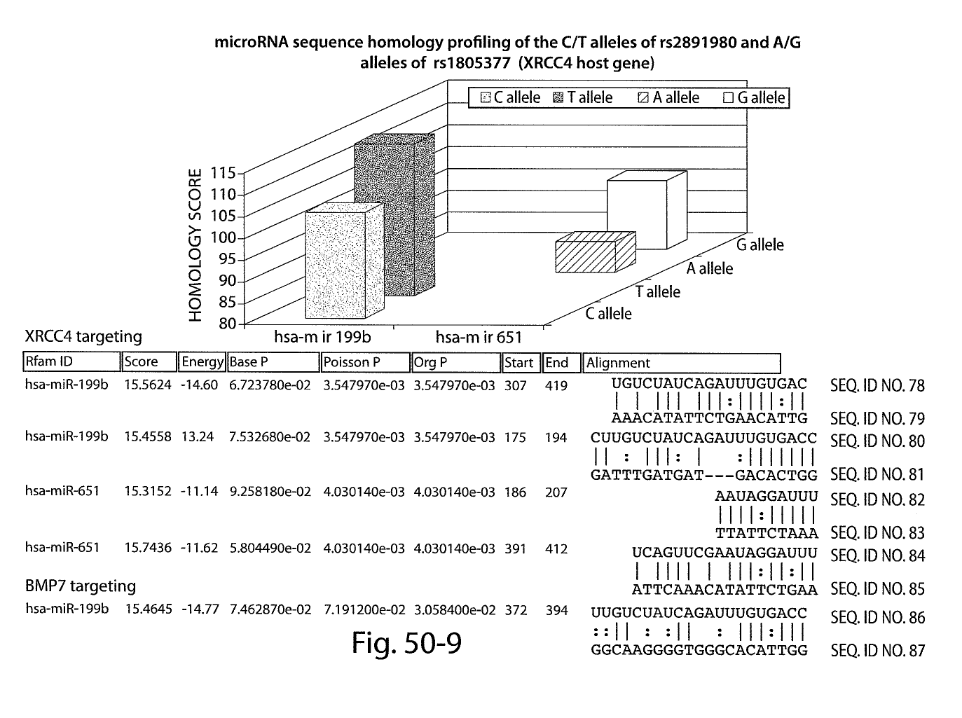

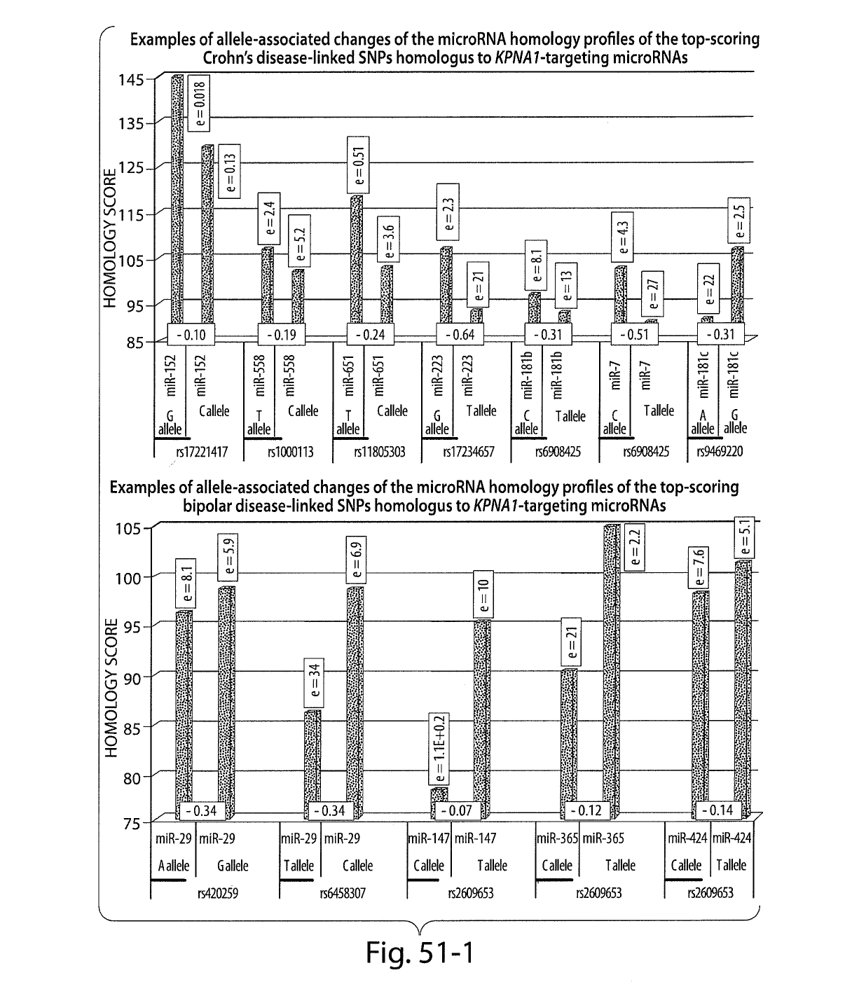

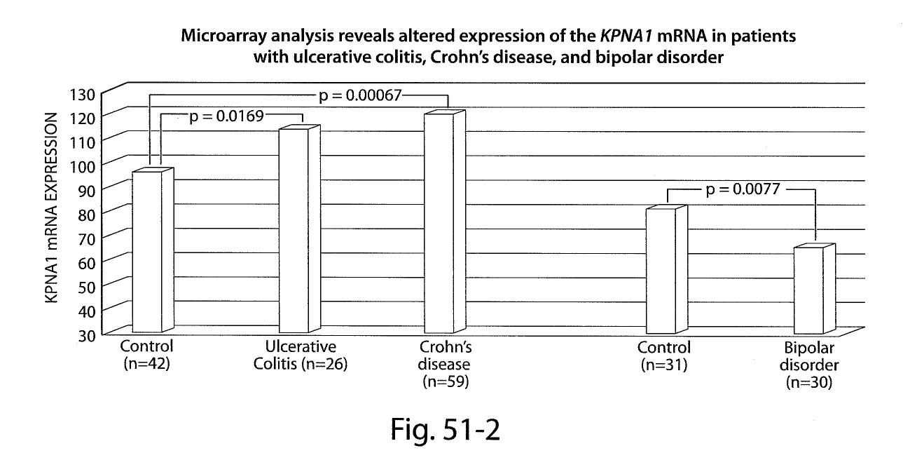

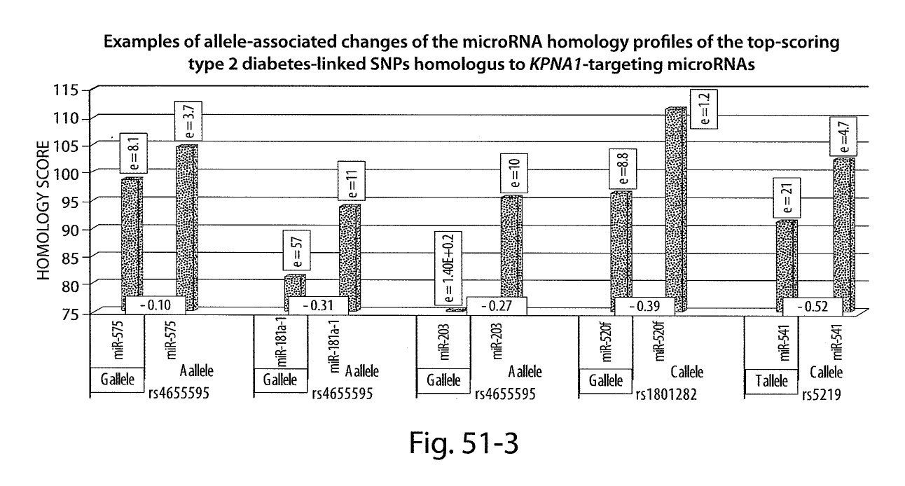

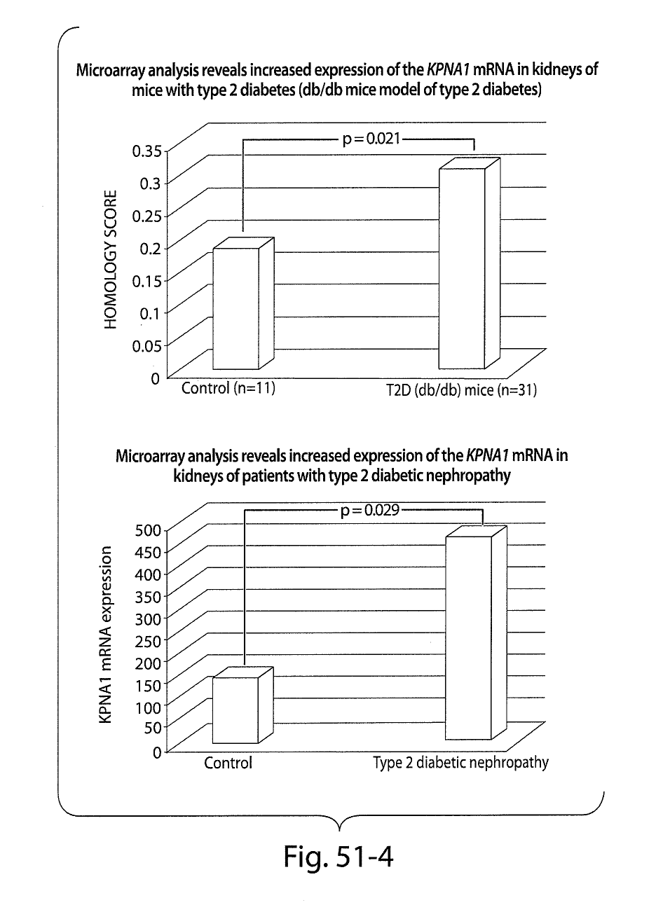

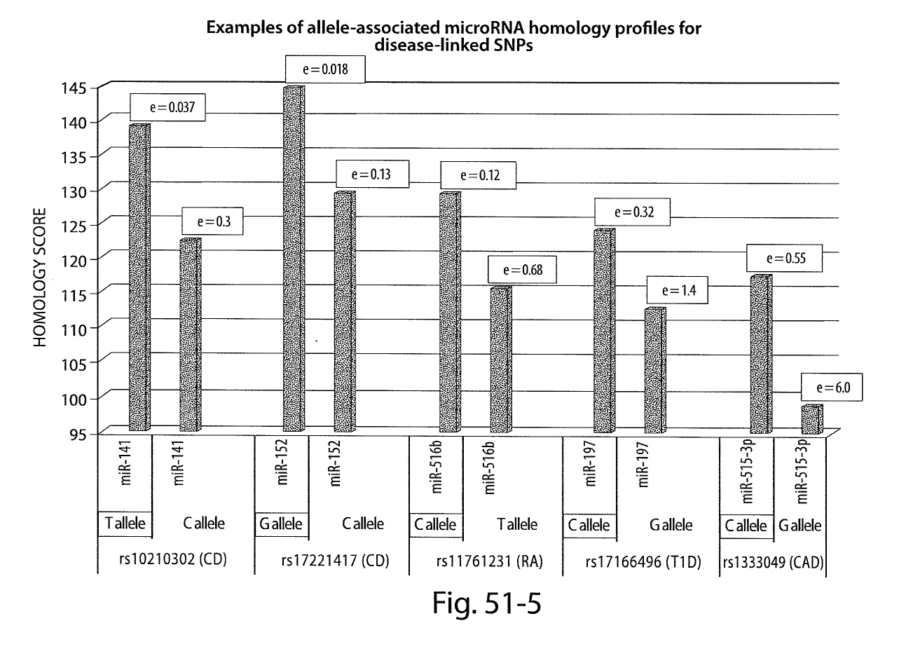

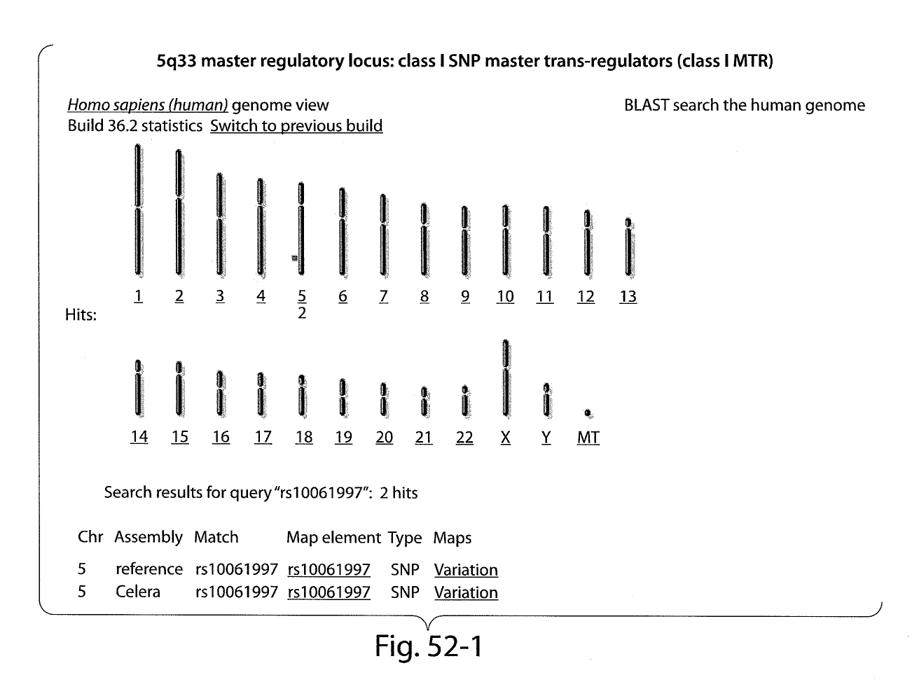

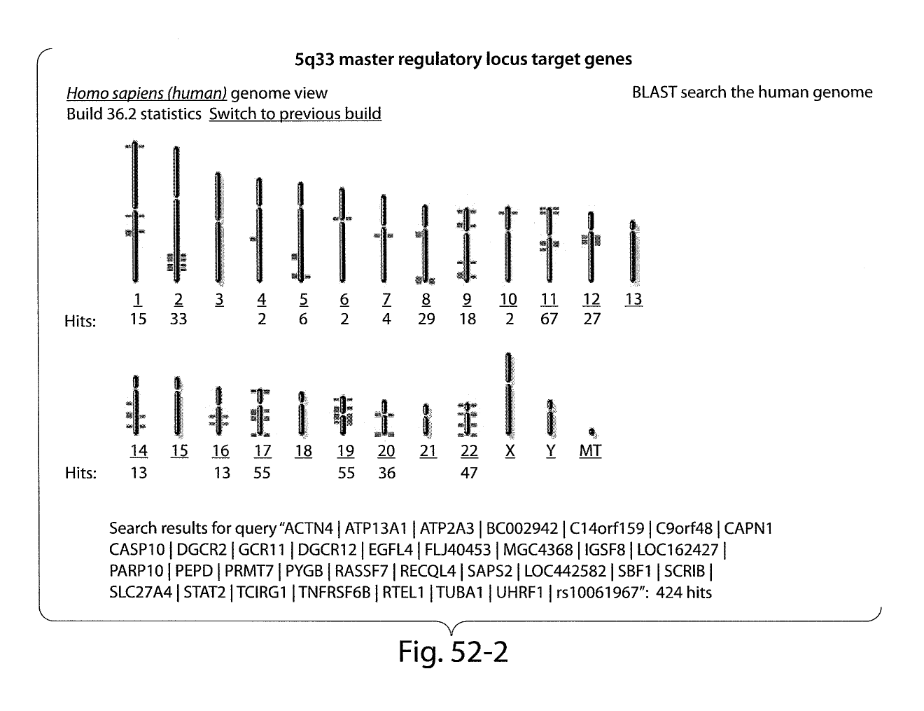

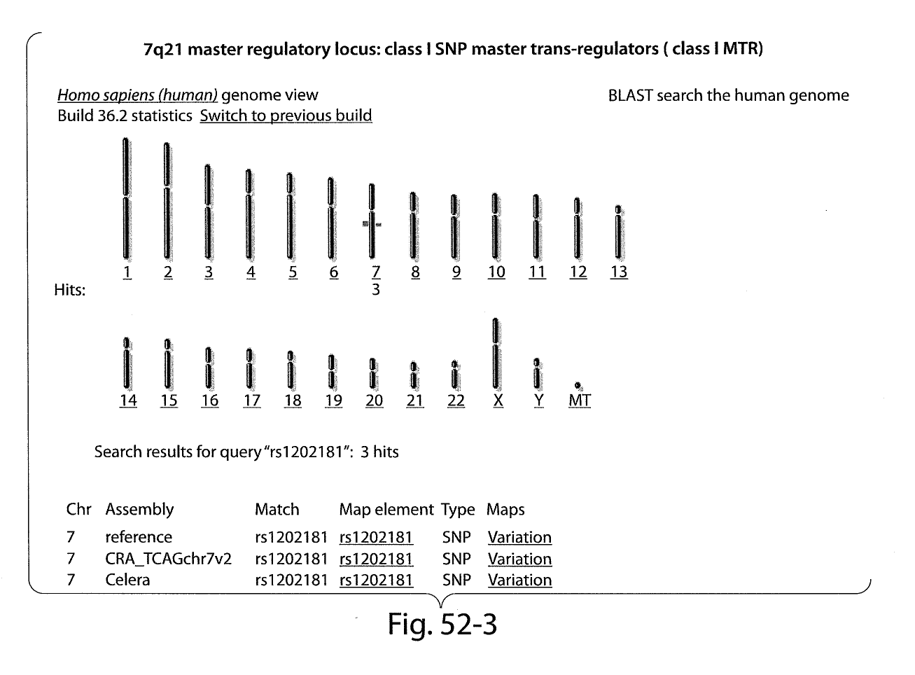

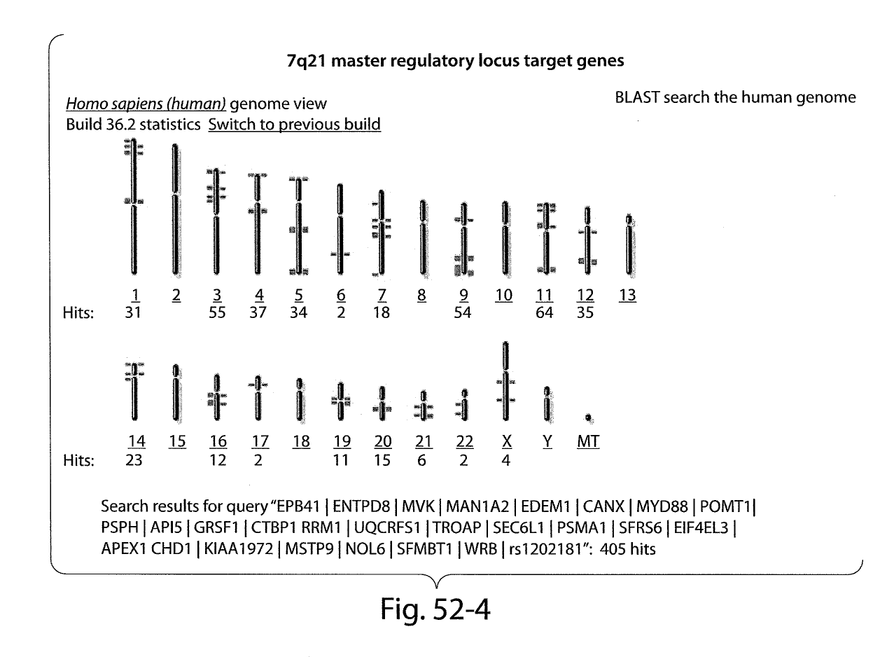

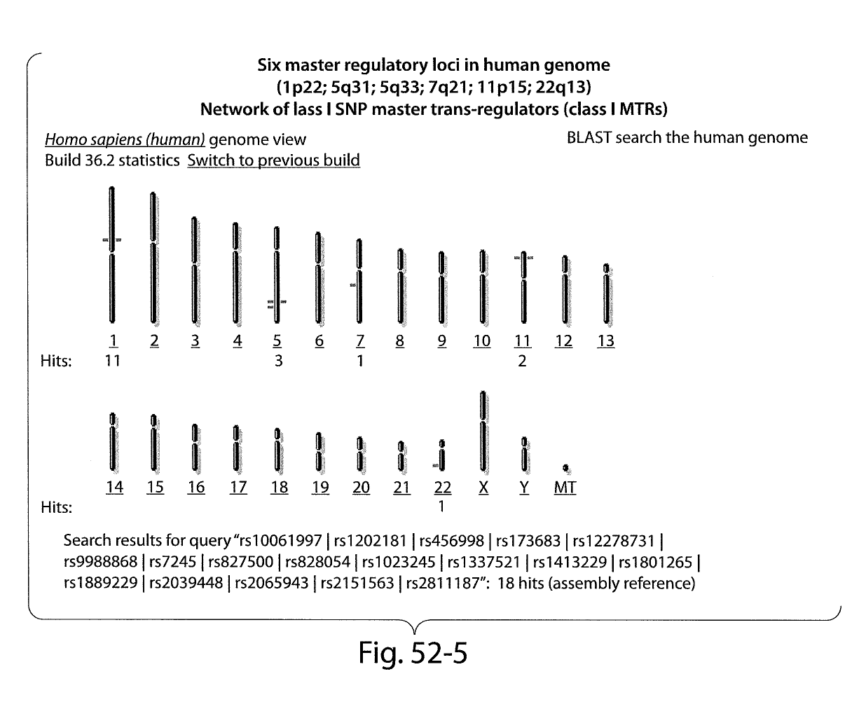

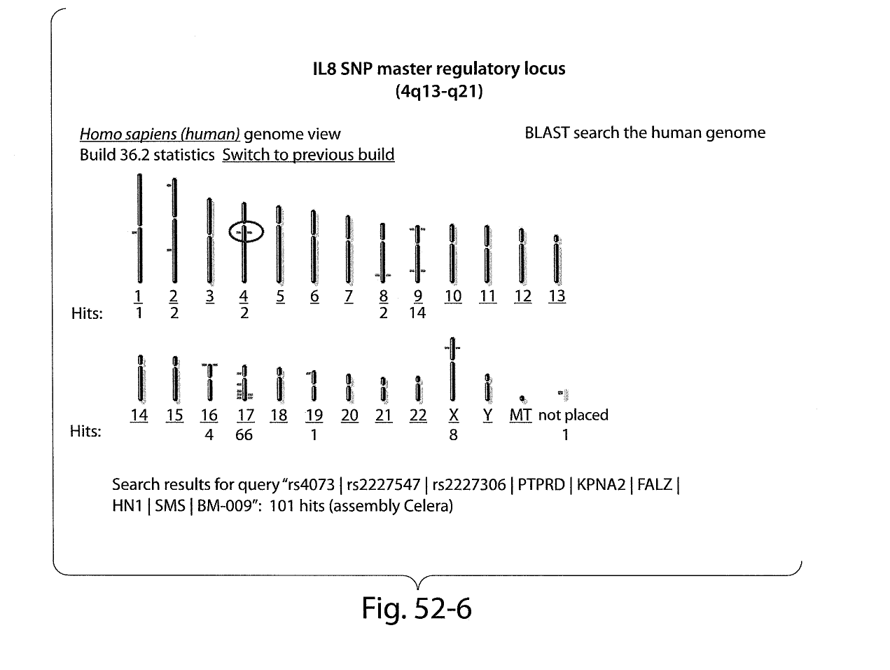

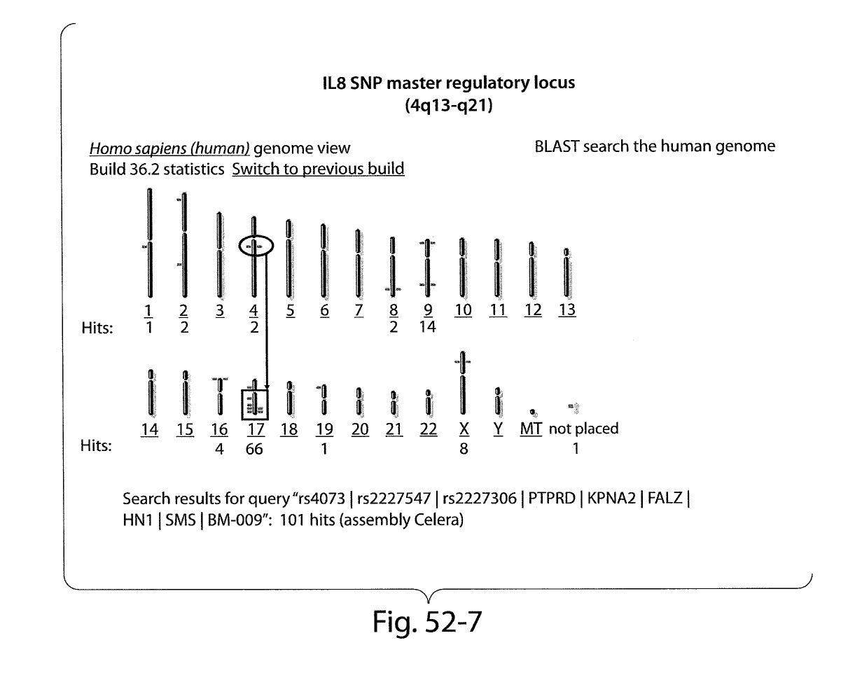

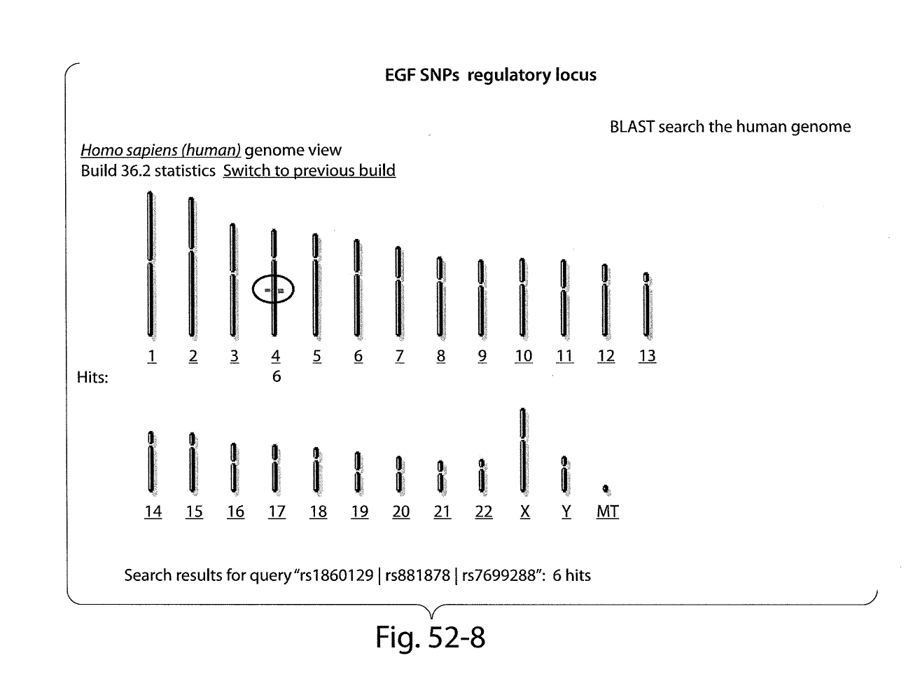

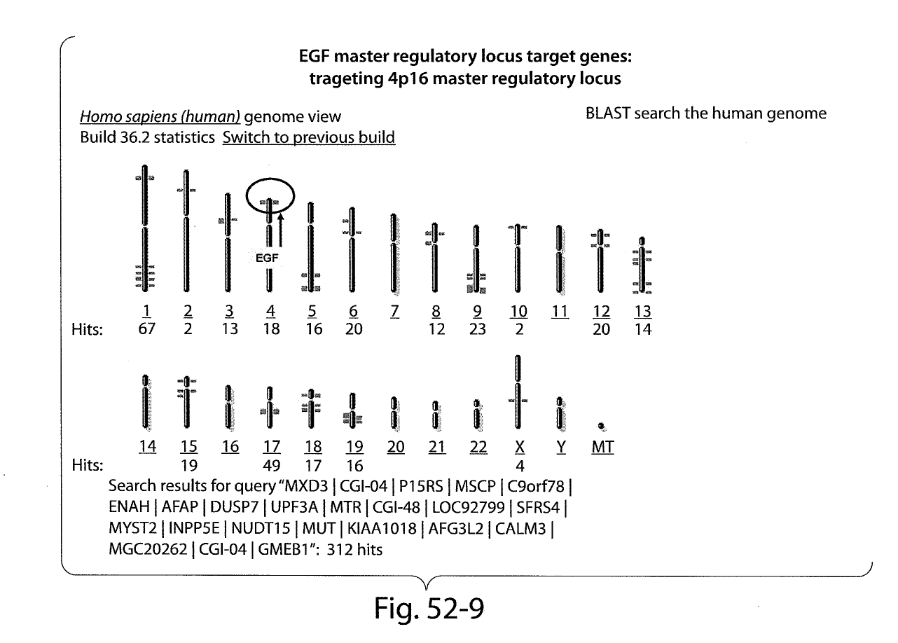

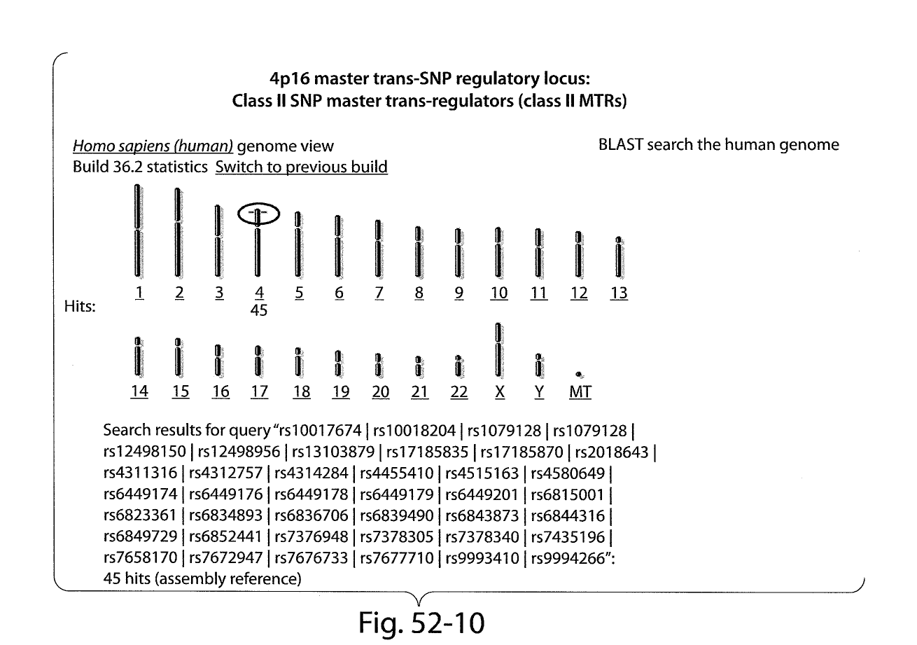

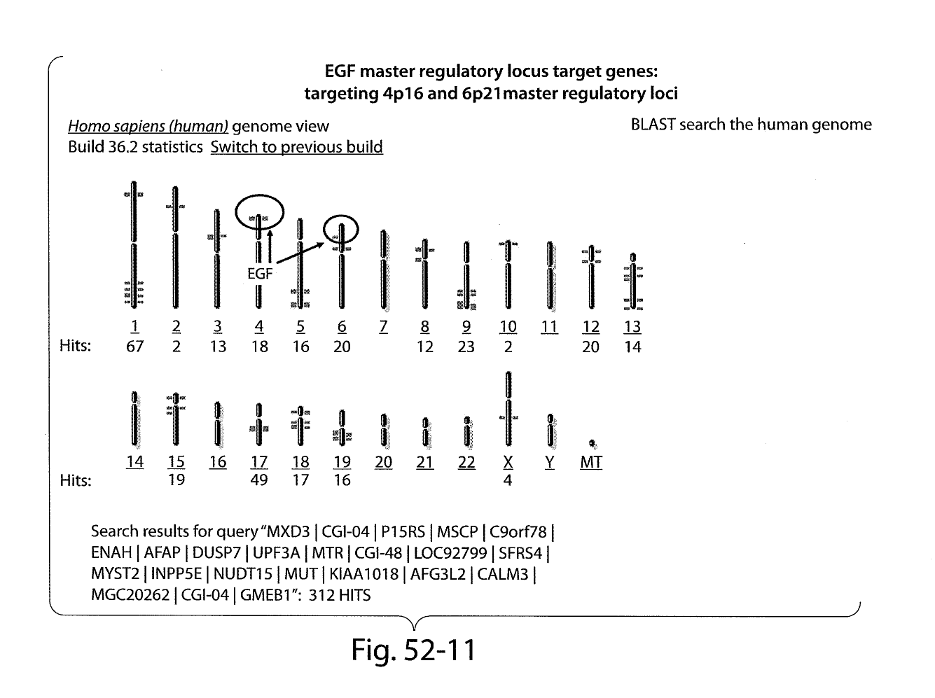

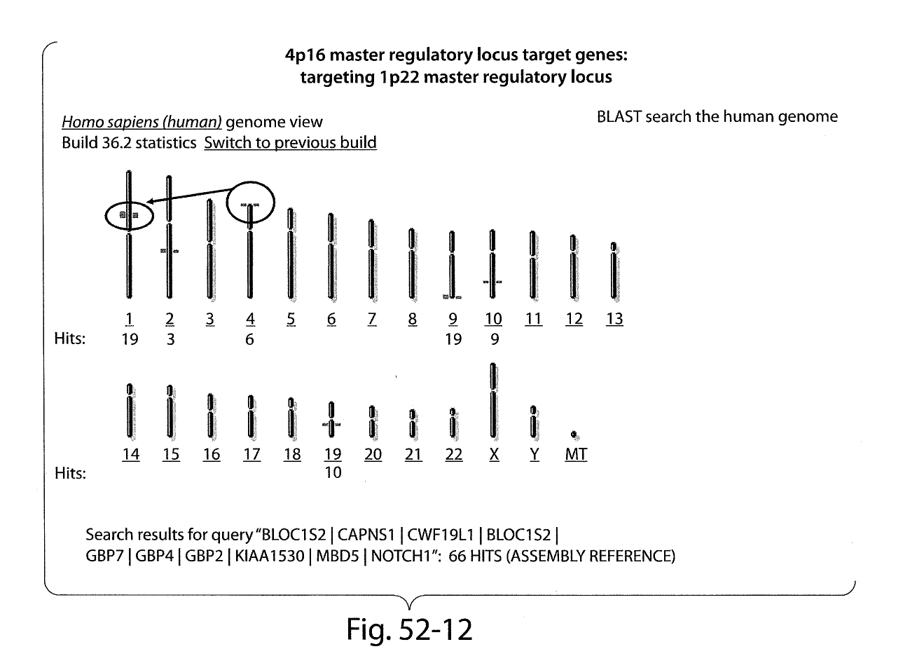

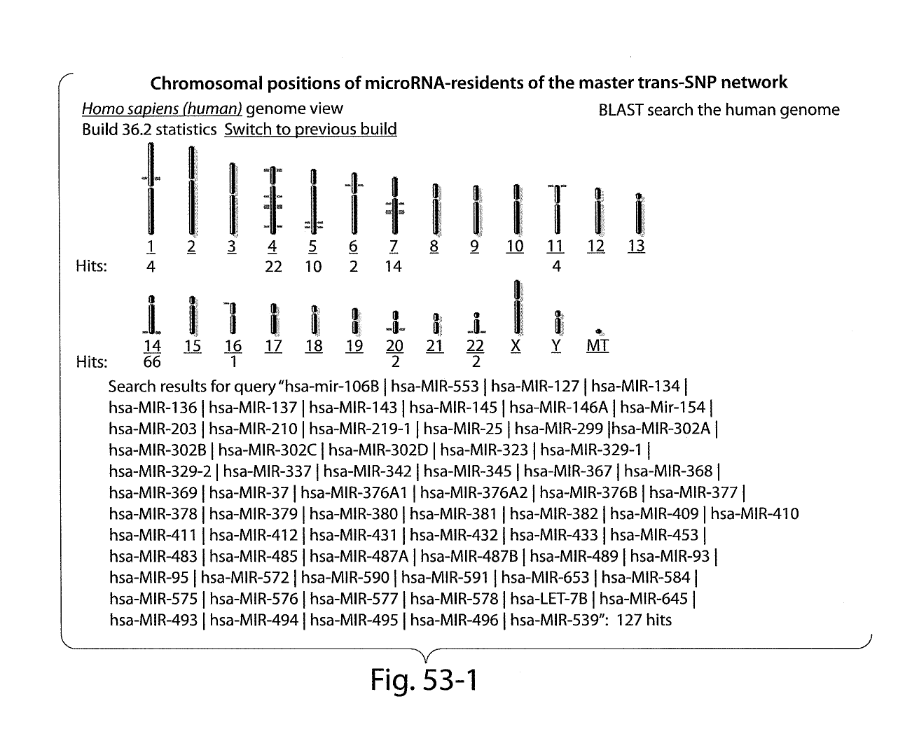

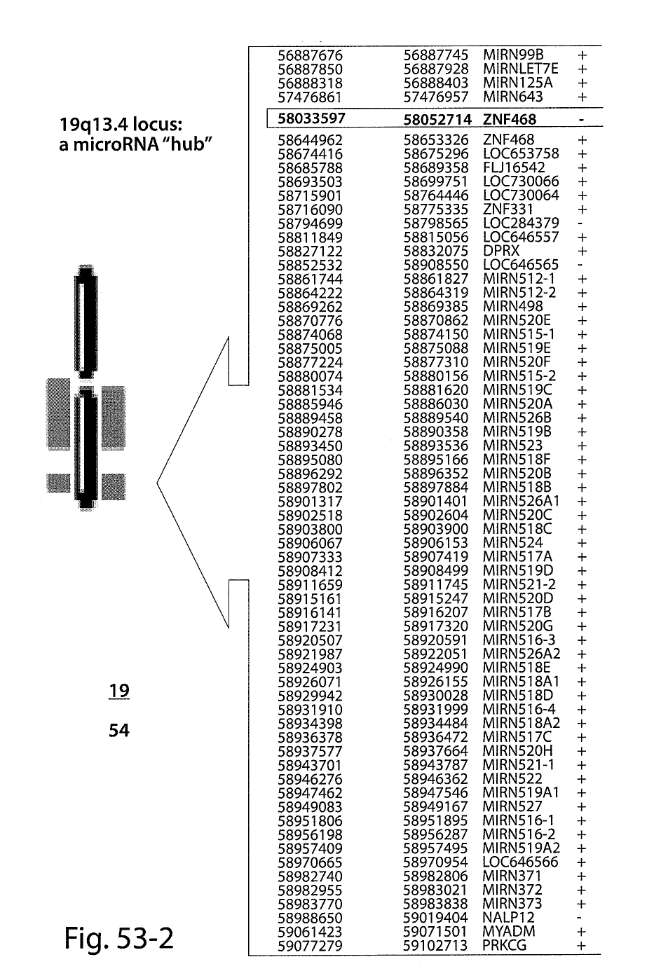

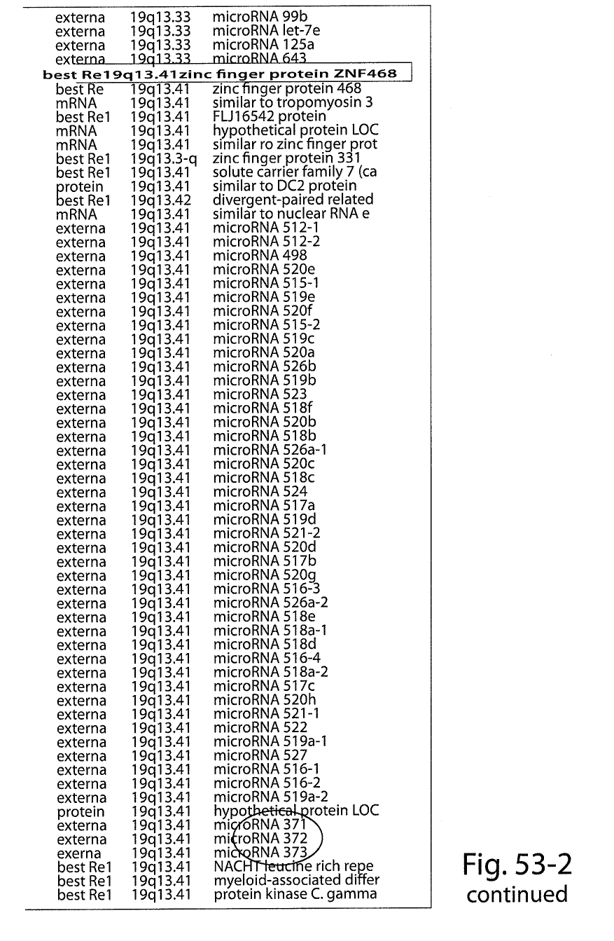

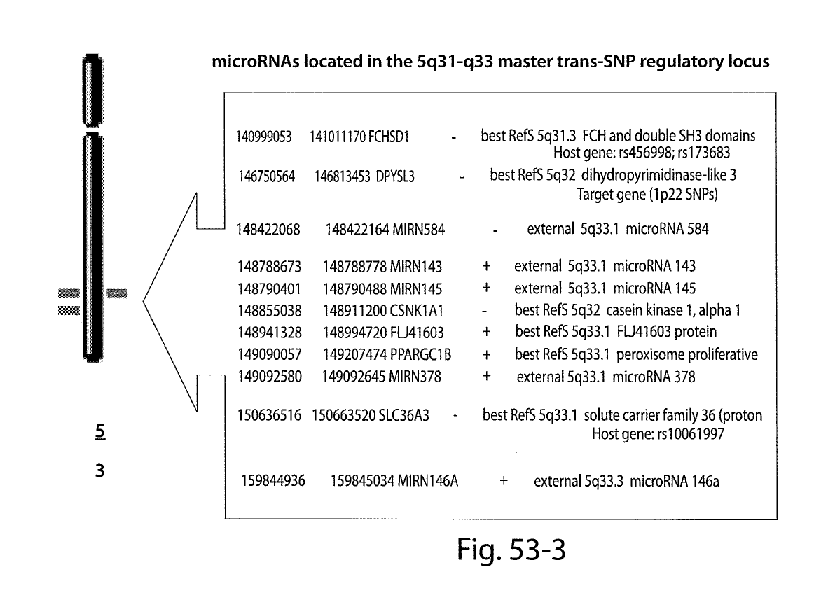

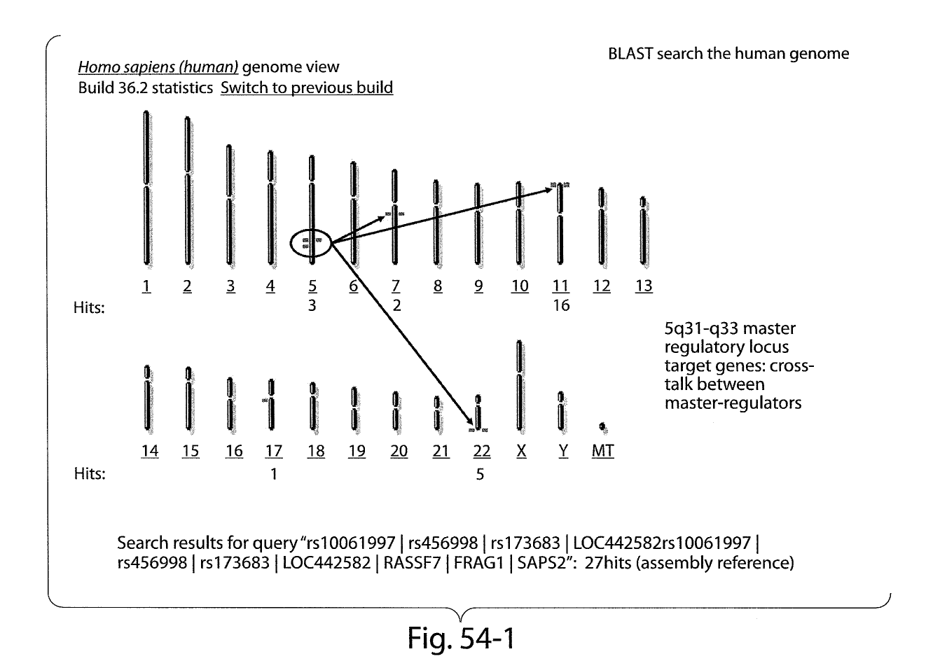

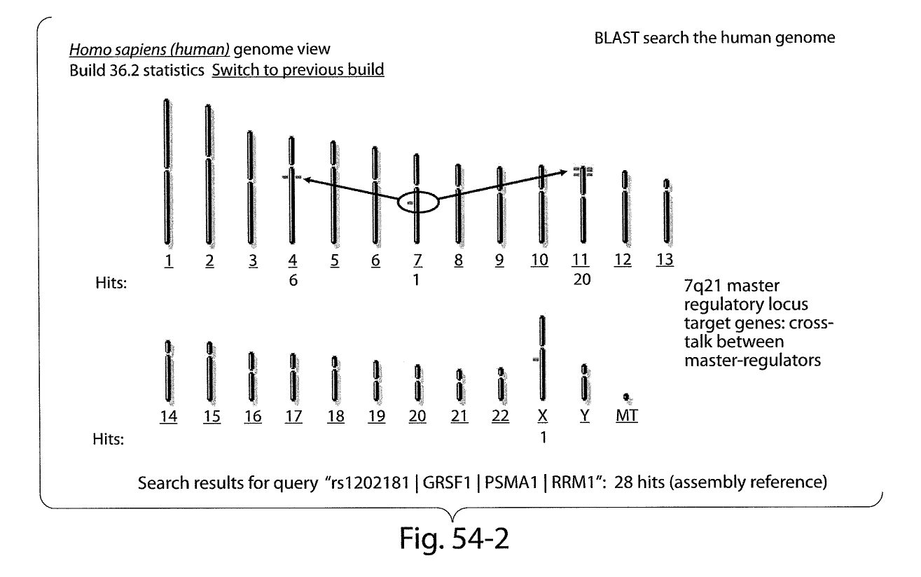

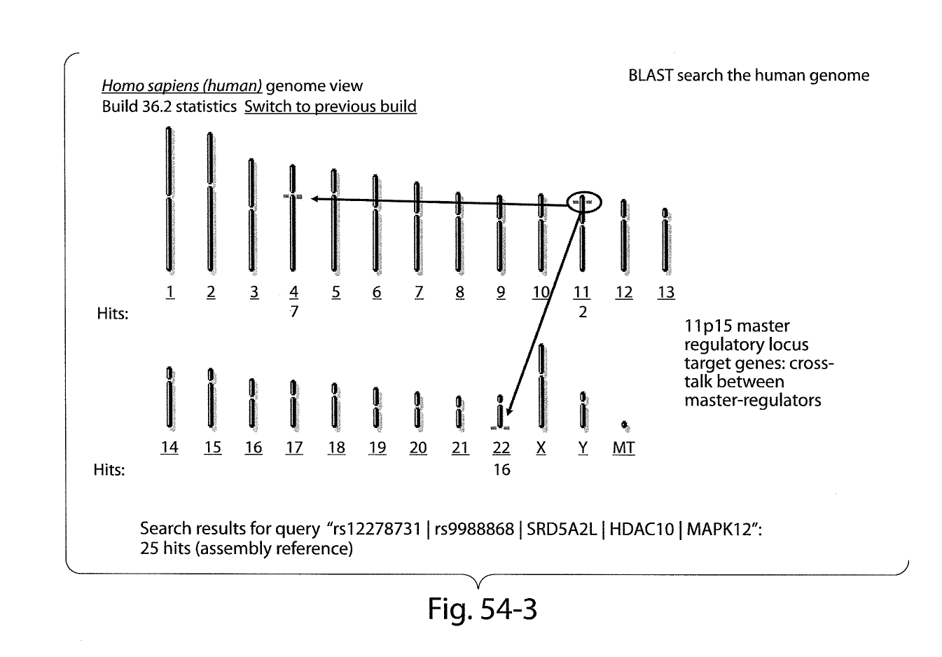

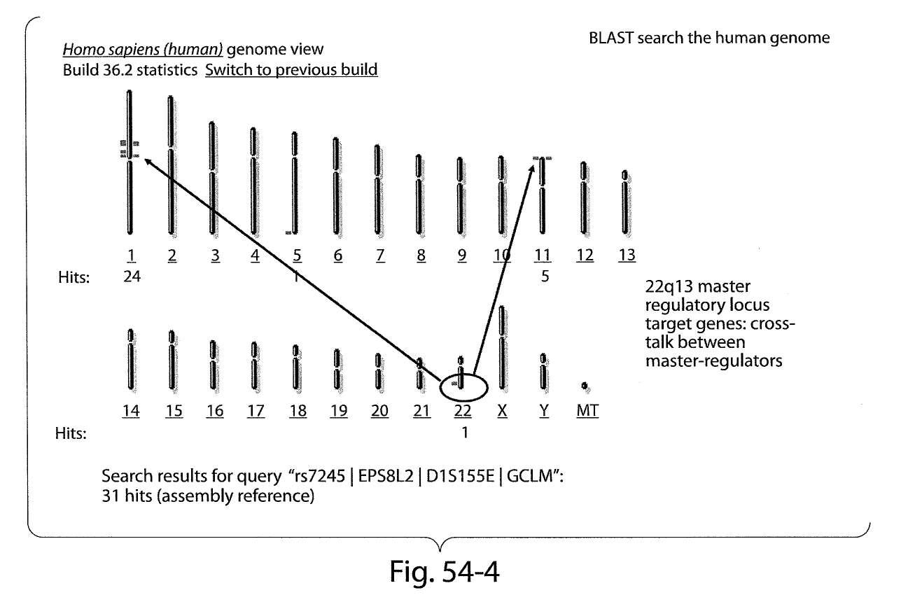

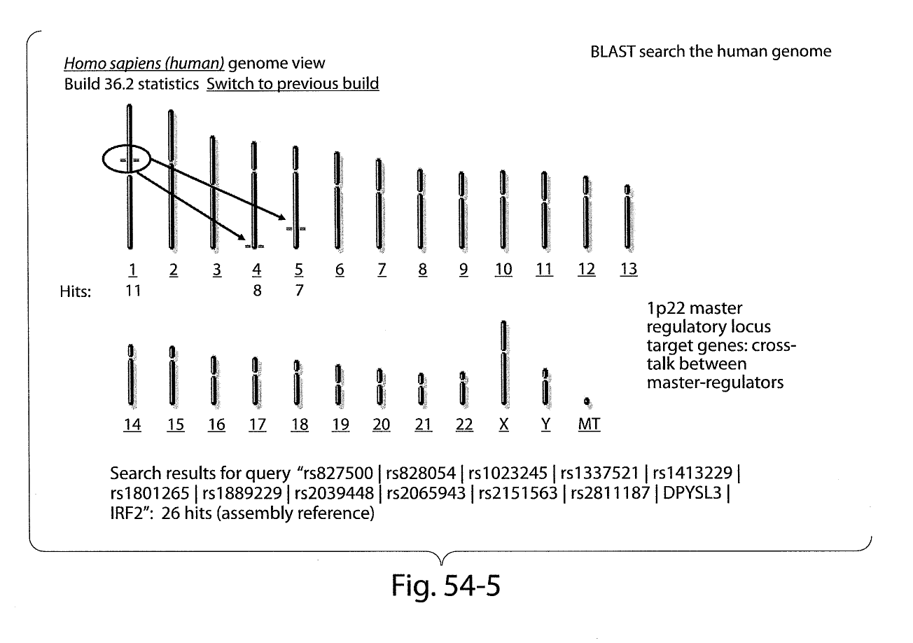

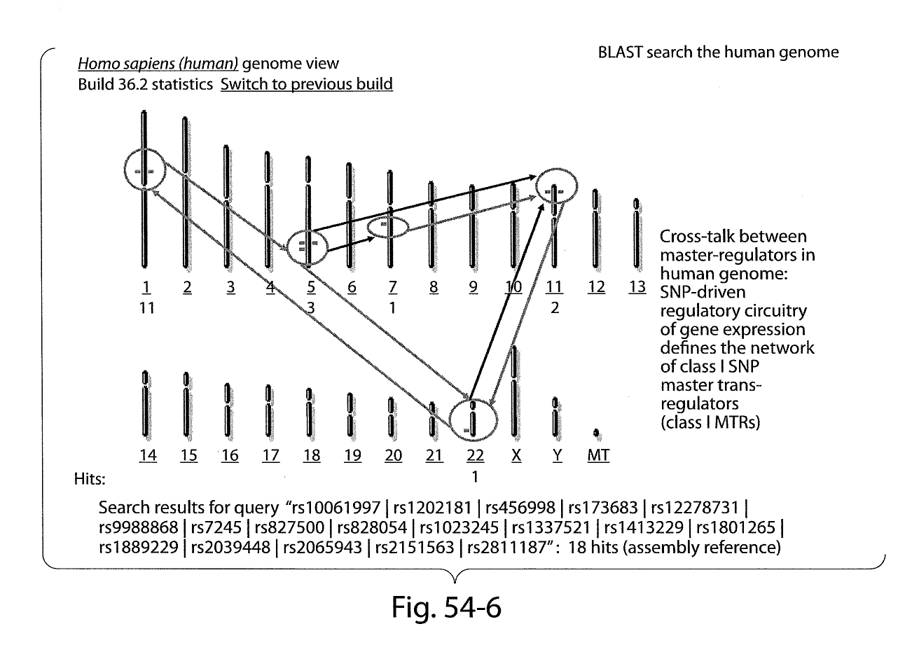

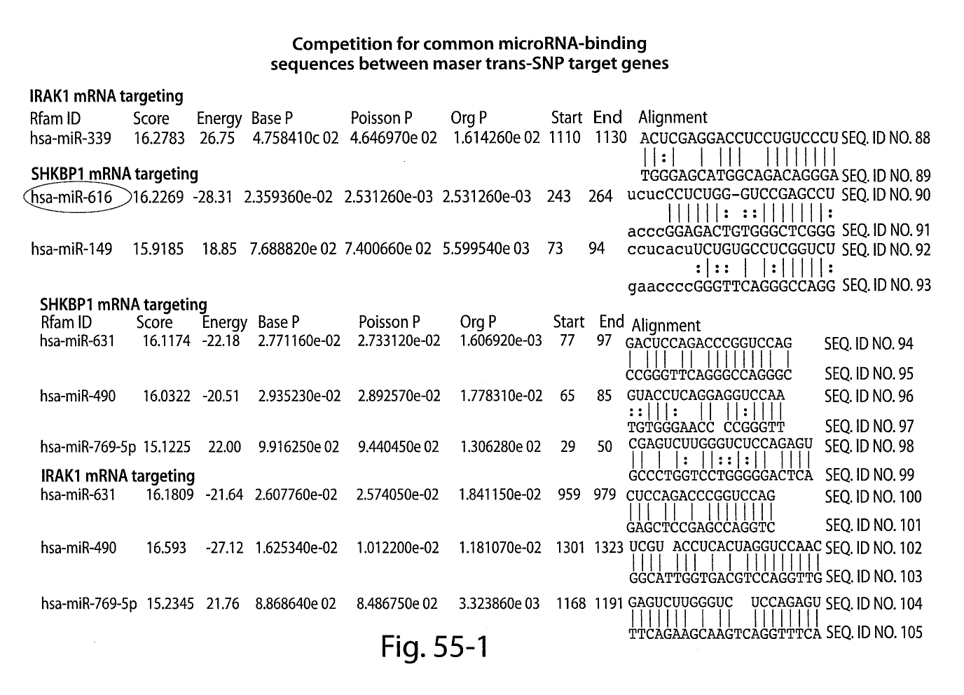

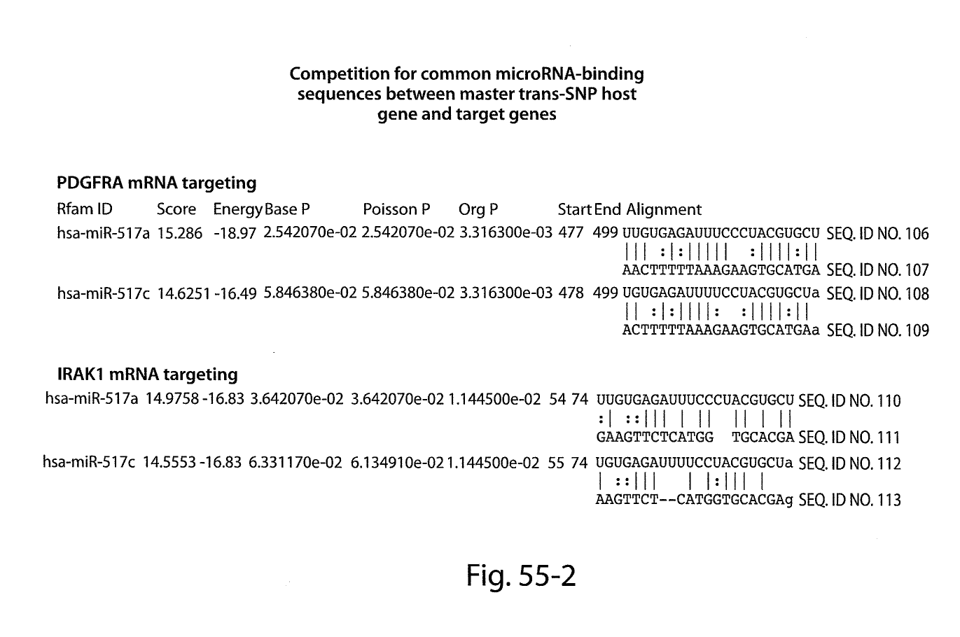

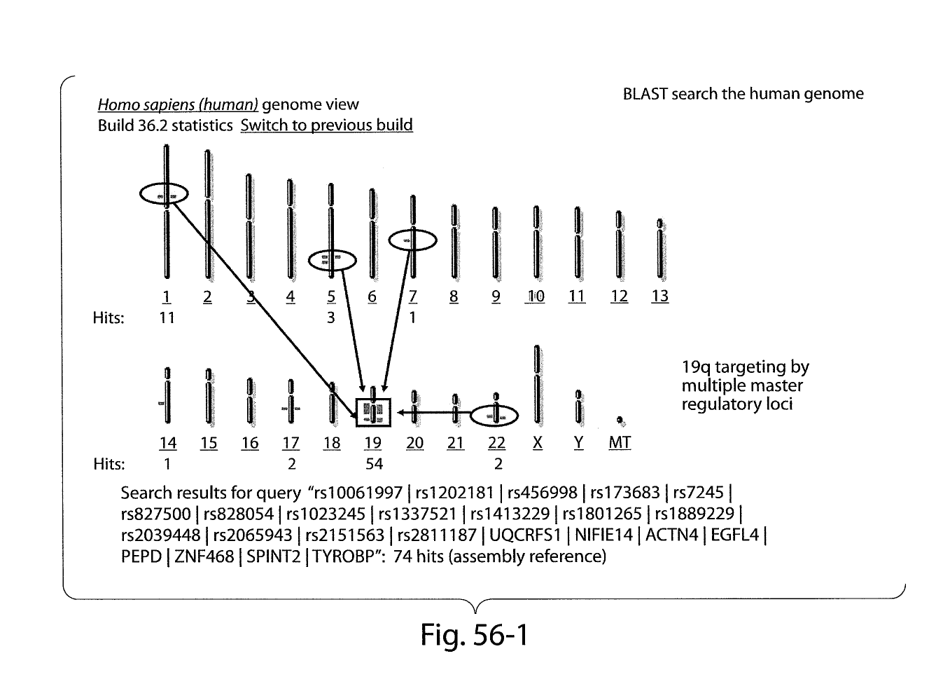

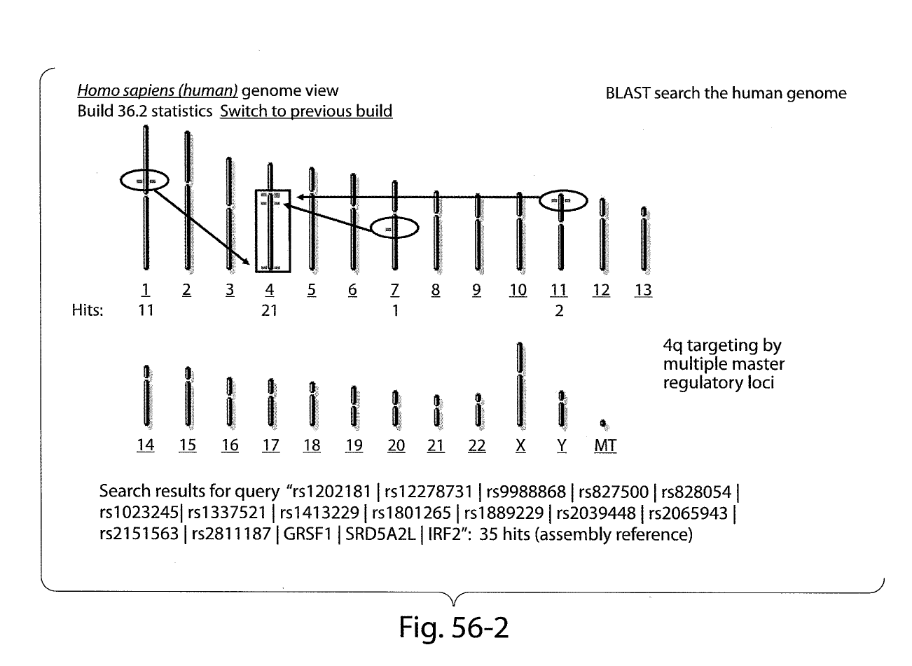

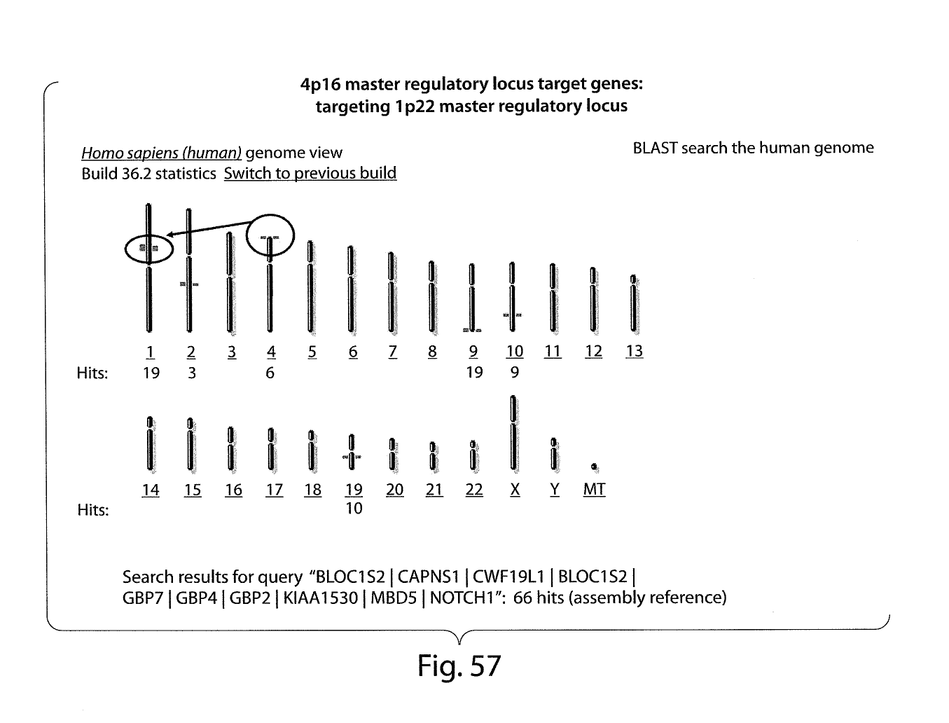

Practical implementation of the disease phenocode concept offers unique opportunities for development of a new family of blockbuster drugs with potential broad clinical utility across the large spectrum of common human disorders. In addition, applications of the disease phenocode concept to individual human disorders can create a net of roadmaps to personalized health care management specifically tailored to genetically-defined diagnosis of pathological conditions and individual's disease profile. Specific examples of implementation of the disease phenocode concept to individual human disorders are outlined below. (See, e.g. FIGS. 21-23 and 27-47). In one example, the type 2 diabetes super MirMap shown in FIG. 38, includes only top-scoring SNPs and microRNAs, i.e. only those that manifest most sequence homology or complementarity events and it represents a subset of SNPs and microRNAs shown in complete type 2 diabetes MirMap, which includes all identified SNPs and microRNAs.

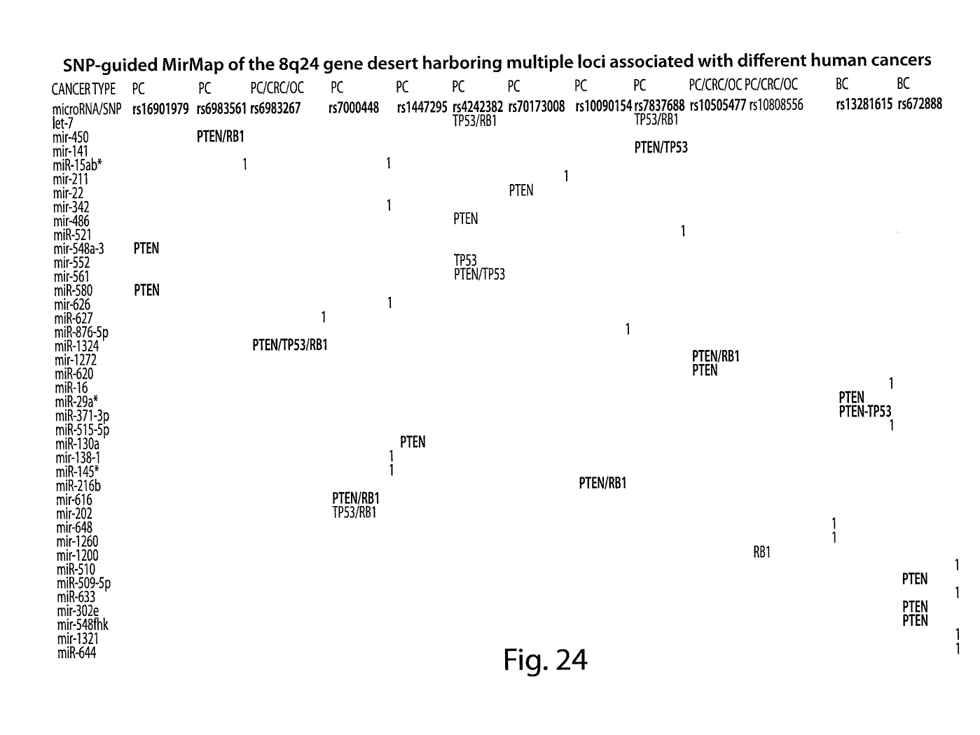

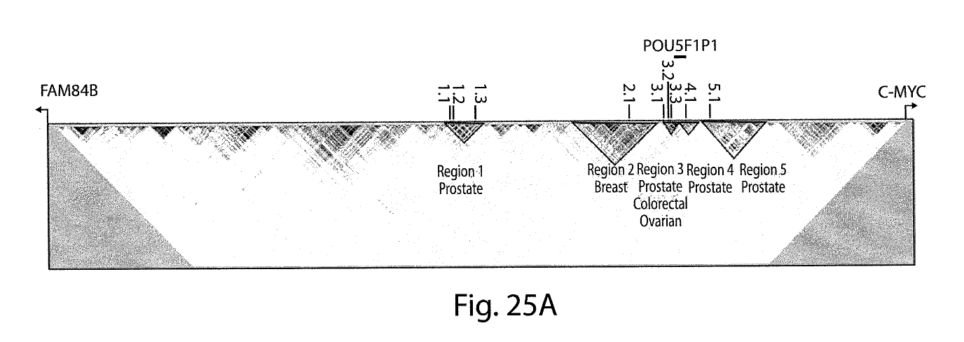

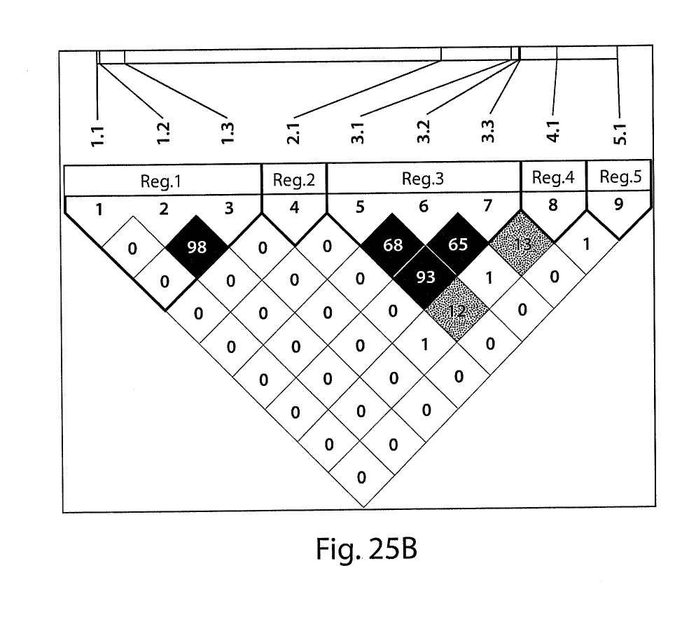

Multiple loci with different cancer specificities within the 8q24 gene desert have been determined. (See FIG. 24). Recent studies based on genome-wide association, linkage, and admixture scan analysis have reported associations of various genetic variants in 8q24 with susceptibility to breast, prostate, and colorectal cancer. This locus lies within a 1.18 MB region that contains no known genes but is bounded at its centromeric end by FAM84B and at its telomeric end by c-MYC, two candidate cancer susceptibility genes. (See FIG. 25). To investigate the associations of specific loci within 8q24 with specific cancers, the nine previously reported cancer-associated single-nucleotide polymorphisms across the region in four case-control sets of prostate (1854 case subjects and 1894 control subjects), breast (2270 case subjects and 2280 control subjects), colorectal (2299 case subjects and 2284 control subjects), and ovarian (1975 case subjects and 3411 control subjects) cancer were genotyped. Five different haplotype blocks within this gene desert were specifically associated with risks of different cancers. One block was solely associated with risk of breast cancer, three others were associated solely with the risk of prostate cancer, and a fifth was associated with the risk of prostate, colorectal, and ovarian cancer, but not breast cancer. Thus there are at least five separate functional variants in this region. Table 16 shows the association of 8q24 single nucleotide polymorphisms with colorectal, ovarian, breast and prostate cancers.

TABLE 16

| | Reference | | | | | | | | |
|---|---|---|---|---|---|---|---|---|---|
| Marker SNP | allele | | | | | | | | |
| (region, | (frequency | | | | | | | | |
| relative | in controls | Colorectal cancer† | | Ovarian cancer | | Breast cancer | | Prostate cancer | |
| position) | subjects) | OR (95% CI) | P value‡ | OR (95% CI) | P value‡ | OR (95% CI) | P value‡ | OR (95% CI) | P value‡ |
| rs13254738 | A (0.70) | 1.06 | 0.22 | 1.02 | 0.64 | 0.96 | 0.35 | 1.12 | 0.029 |
| (A/C) (1.1) | | (0.99 to 1.13) | | (0.94 to 1.11) | | (0.88 to 1.05) | | (1.01 to 1.24) | |
| (region 1, | | | | | | | | | |
| 128173525) | | | | | | | | | |
| rs6983561 | A (0.97) | 0.95 | 0.65 | 0.90 | 0.36 | 0.96 | 0.76 | 2.11 | 1.4 × 10⁻ [symbol] |
| (A/C) (1.2) | | (0.81 to 1.11) | | (0.72 to 1.13) | | (0.77 to 1.21) | | (1.65 to 2.71) | |
| (region 1, | | | | | | | | | |
| 128176062) | | | | | | | | | |
| rs16901979 | G (0.97) | 0.89 | 0.36 | 0.89 | 0.30 | 0.98 | 0.98 | 2.06 | 4.9 × 10⁻ [symbol] |
| (G/T) (1.3) | | (0.77 to 1.06) | | (0.71 to 1.11) | | (0.80 to 1.25) | | (1.61 to 2.65) | |
| (region 1, | | | | | | | | | |
| 128194098) | | | | | | | | | |
| rs13281615 | A (0.80) | 0.94 | 0.17 | 0.99 | 0.75 | 1.21 | 1 × 10⁻ [symbol] | 0.95 | 0.33 |
| (A/G) (2.1) | | (0.89 to 1.00) | | (0.91 to 1.07) | | (1.11 to 1.32) | | (0.87 to 1.05) | |
| (region 2, | | | | | | | | | |
| 128424800)§ | | | | | | | | | |
| rs10505477 | G (0.50) | 1.27 | 2.9 × 10⁻ [symbol] | 1.14 | 2.0 × 10⁻ [symbol] | 0.96 | 0.35 | 1.43 | 7.7 × 10⁻ [symbol] |
| (G/A) (3.1) | | (1.19 to 1.33) | | (1.04 to 1.23) | | (0.88 to 1.04) | | (1.30 to 1.56) | |
| (region 3, | | | | | | | | | |
| 128476625) | | | | | | | | | |
| rs10808556 | A (0.59) | 1.26 | 5.1 × 10⁻ [symbol] | 1.13 | 1.7 × 10⁻ [symbol] | 0.99 | 0.80 | 1.31 | 4.2 × 10⁻ [symbol] |
| (A/G) (3.2) | | (1.16 to 1.37) | | (1.04 to 1.22) | | (0.91 to 1.09) | | (1.19 to 1.44) | |
| (region 3, | | | | | | | | | |
| 128482329) | | | | | | | | | |
| rs6983267 [symbol] | A (0.49) | 1.27 | 3.6 × 10⁻ [symbol] | 1.11 | 9.9 × 10⁻ [symbol] | 0.97 | 0.50 | 1.43 | 7.7 × 10⁻ [symbol] |
| (A/G) (3.3) | | (1.16 to 1.37) | | (1.03 to 1.20) | | (0.89 to 1.05) | | (1.30 to 1.56) | |
| (region 3, | | | | | | | | | |
| 128482487)§ | | | | | | | | | |
| rs7000449 | G (0.64) | 1.04 | 0.32 | 1.04 | 0.33 | 0.96 | 0.38 | 1.23 | 2.8 × 10⁻⁵ |
| (G/A) (4.1) | | (0.98 to 1.11) | | (0.96 to 1.13) | | (0.88 to 1.05) | | (1.11 to 1.35) | |
| (region 4, | | | | | | | | | |
| 128510352) | | | | | | | | | |
| rs1447295 | G (0.90) | 0.98 | 0.62 | 1.07 | 0.35 | 0.92 | 0.28 | 1.86 | 6.9 × 10⁻ [symbol] |
| (G/T) (5.1) | | (0.89 to 1.08) | | (0.93 to 1.22) | | (0.80 to 1.07) | | (1.60 to 2.15) | |
| (region 5, | | | | | | | | | |
| 128554220) | | | | | | | | | |

Genotype results in Table 16 were obtained for more than 95% of all subjects. rs10090154 was not evaluated because it was perfectly correlated with rs1447295 in the European population sample. All genotyping was performed by Taqman assay unless otherwise indicated. No deviation from Hardy-Weinberg equilibrium was observed in the genotype distributions of the control subjects for any of the SNPs. OR = odds ratio; CI = confidence interval; SNP = single nucleotide polymorphism. The bold font refers to significant P values (<0.05) and their corresponding OR. The P values were from the Cochran-Armitage trend test. Re6983267 was genotyped using the illumine 550K chip covering approximately 550,000 SNPs across the genome. Therefore, the SNPs were replaced by alternative tags on the illumine chip: rs13281615 by rs672888 (r² = 0.97) and rs10505477 by rs6983267 (r² = 0.93). [symbol]
indicates data missing or illegible when filed

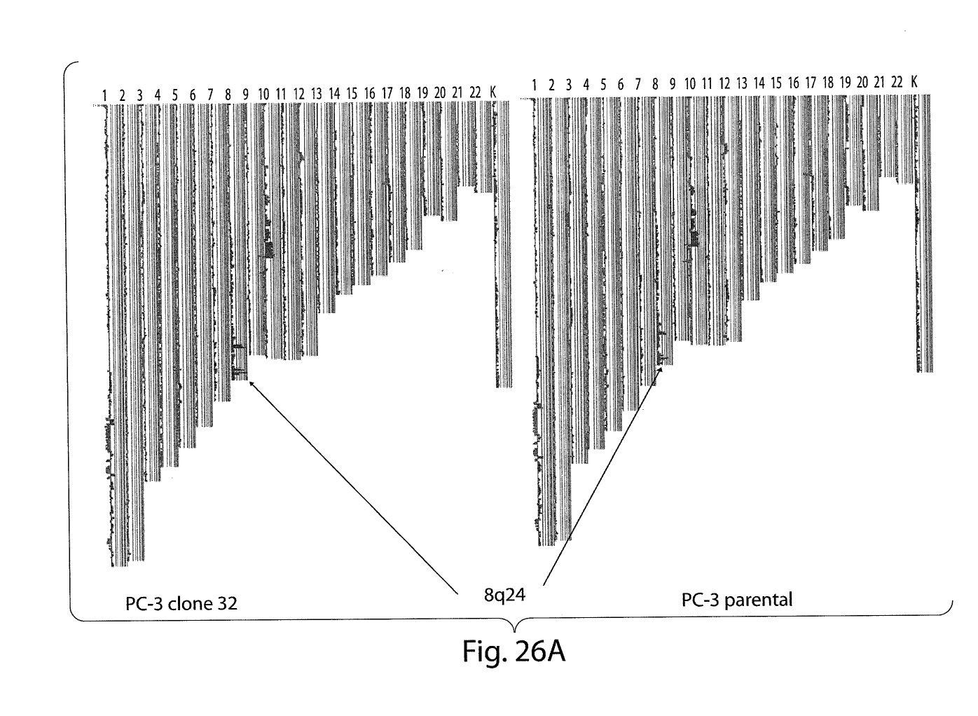

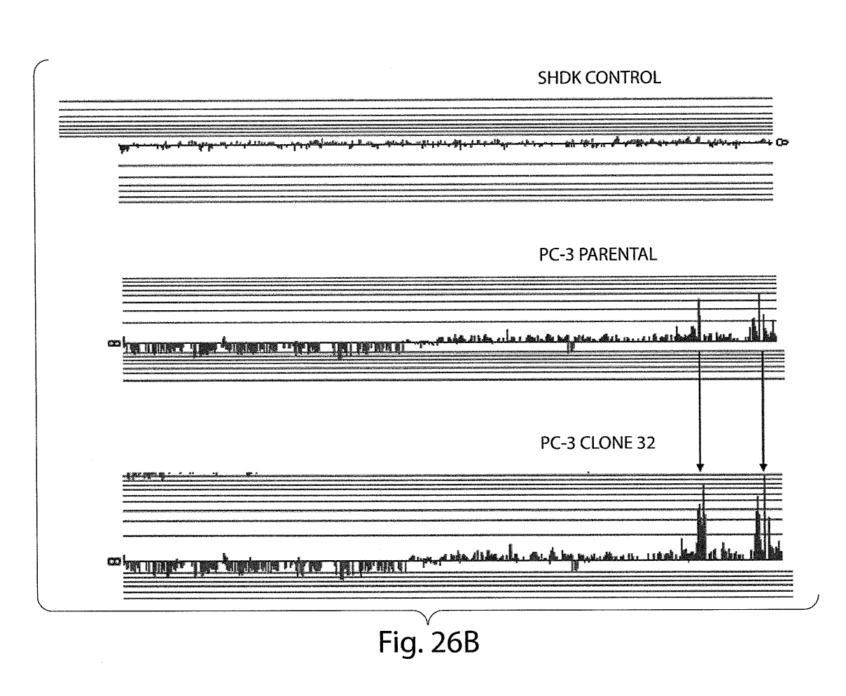

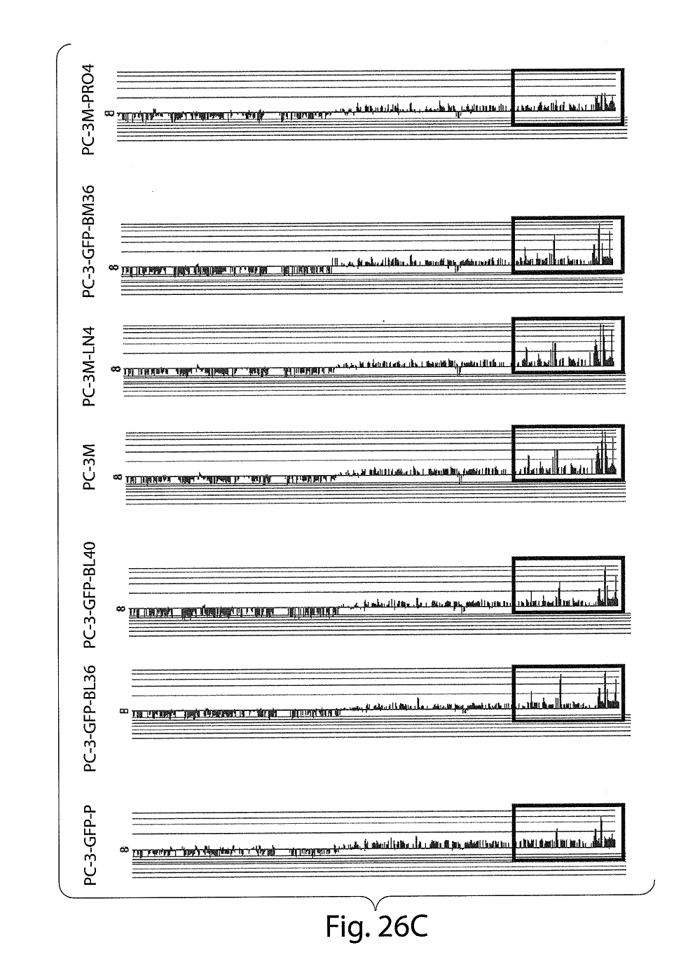

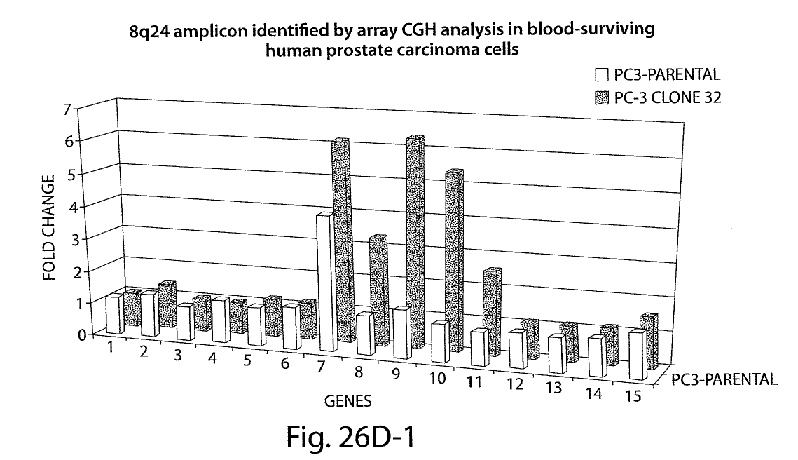

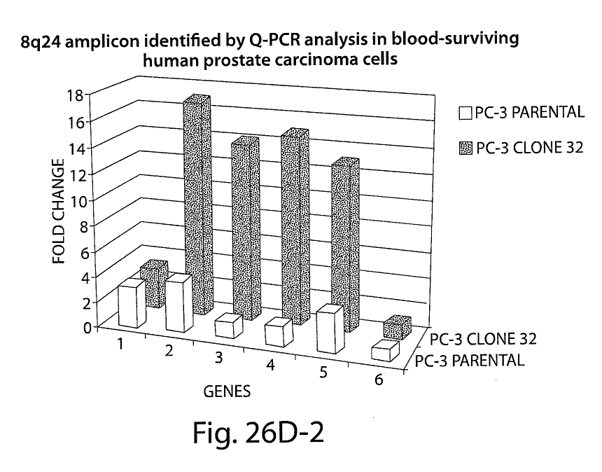

FIG. 26 shows a SNP-guided MirMap of schizophrenia. Reduced fecundity, which is associated with severe mental disorders, places negative selection pressure on risk alleles and may explain, in part, why common variants have not been found that confer risk of disorders such as autism, schizophrenia, and mental retardation. Thus, rare variants may account for a larger fraction of the overall genetic risk than previously assumed. In contrast to rare single nucleotide mutations, rare copy number variations (CNVs) can be detected using genome-wide single nucleotide polymorphism arrays, which has led to the identification of CNVs associated with mental retardation and autism.

In a genome-wide search for CNVs associated with schizophrenia, a population-based sample was used to identify de novo CNVs by analyzing 9,878 transmissions from parents to offspring. The 66 de novo CNVs identified were then tested for association in a sample of 1,433 schizophrenia cases and 33,250 controls. Three deletions at 1q21.1, 15q11.2 and 15q13.3 showing nominal association with schizophrenia in the first sample (phase I) were followed up in a second sample of 3,285 cases and 7,951 controls (phase II). All three deletions significantly associate with schizophrenia and related psychoses in the combined sample.

The identification of these rare, recurrent risk variants, having occurred independently in multiple founders and being subject to negative selection, is important in itself. Moreover, CNV analysis may also point the way to the identification of additional and more prevalent risk variants in genes and pathways involved in schizophrenia.

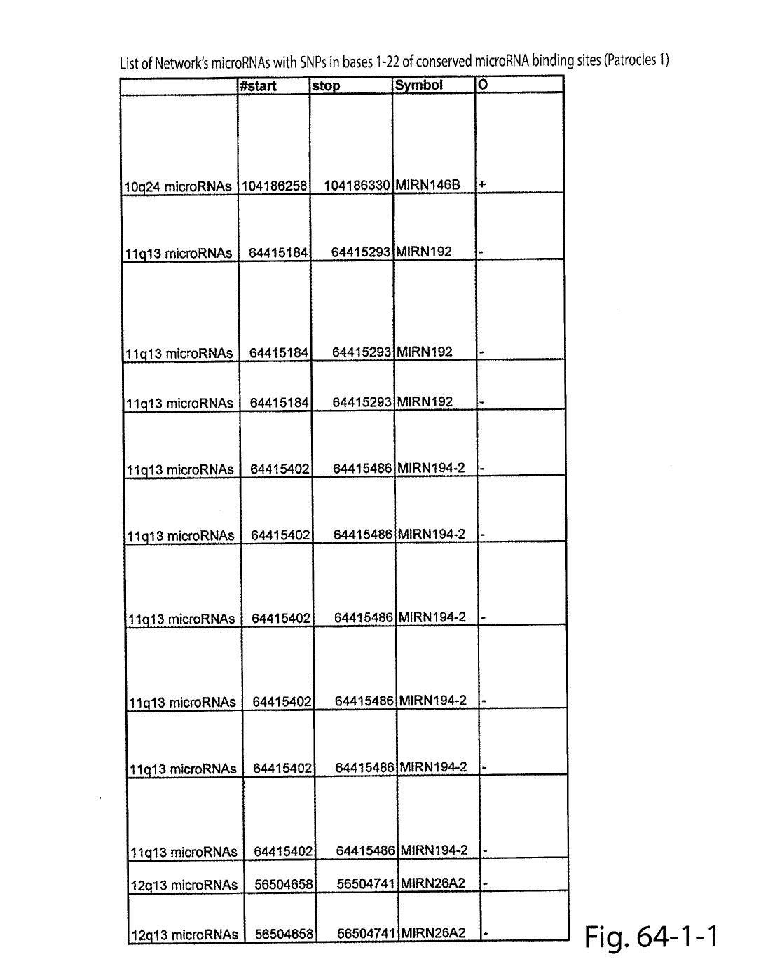

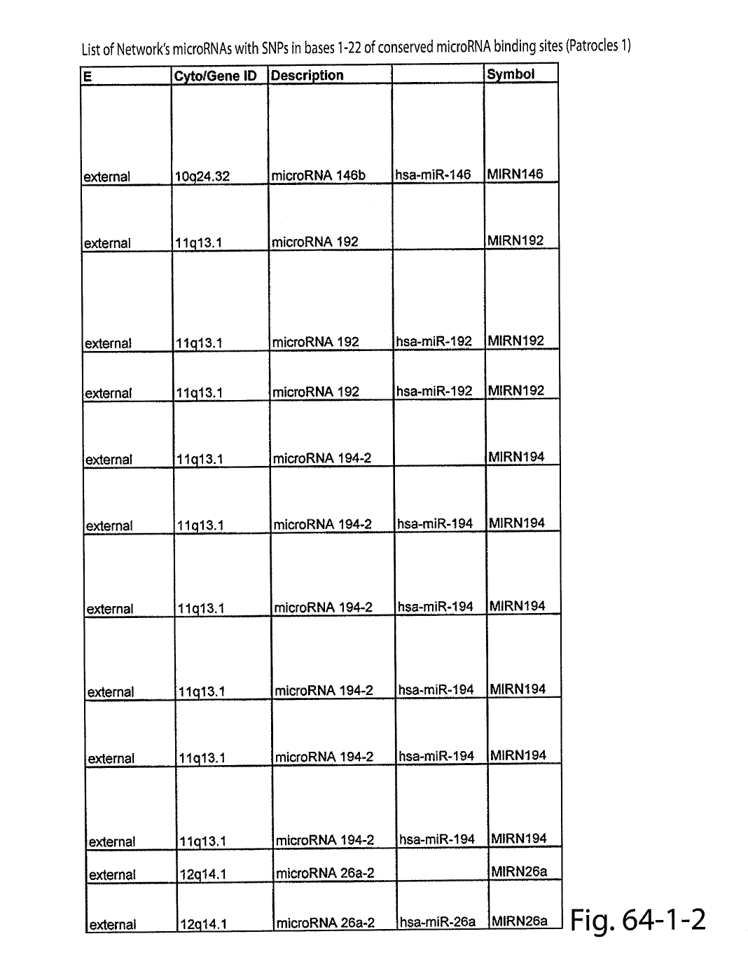

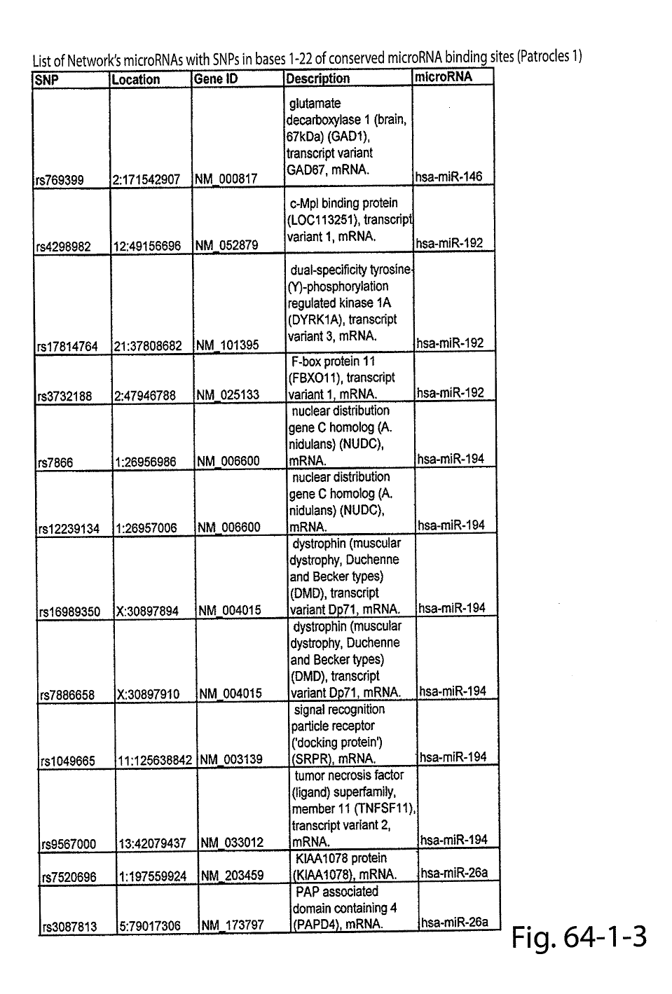

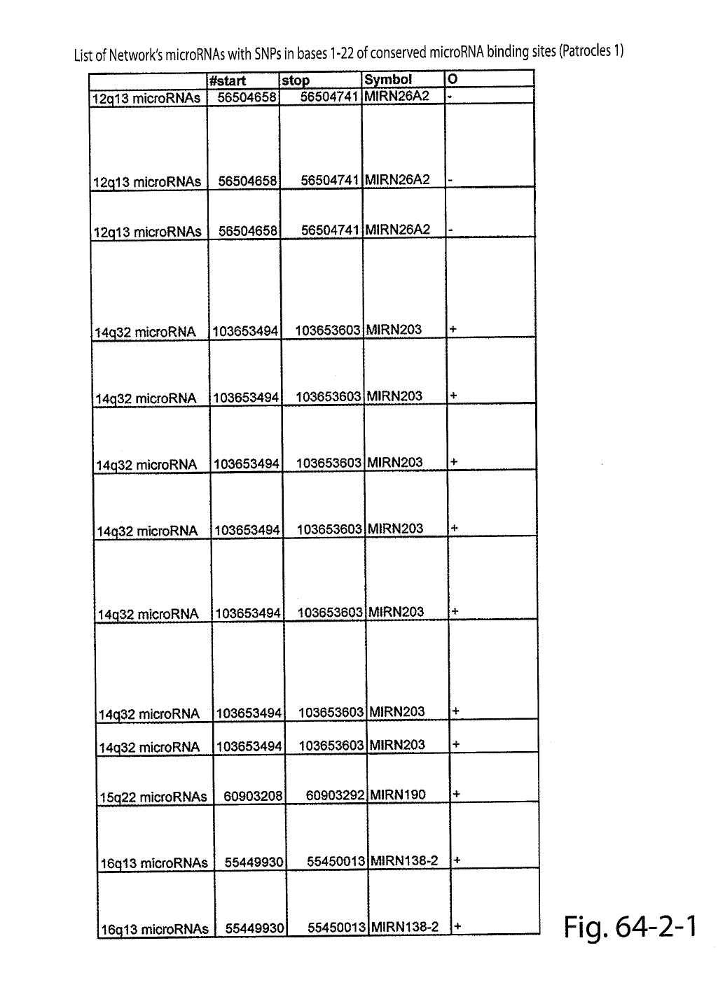

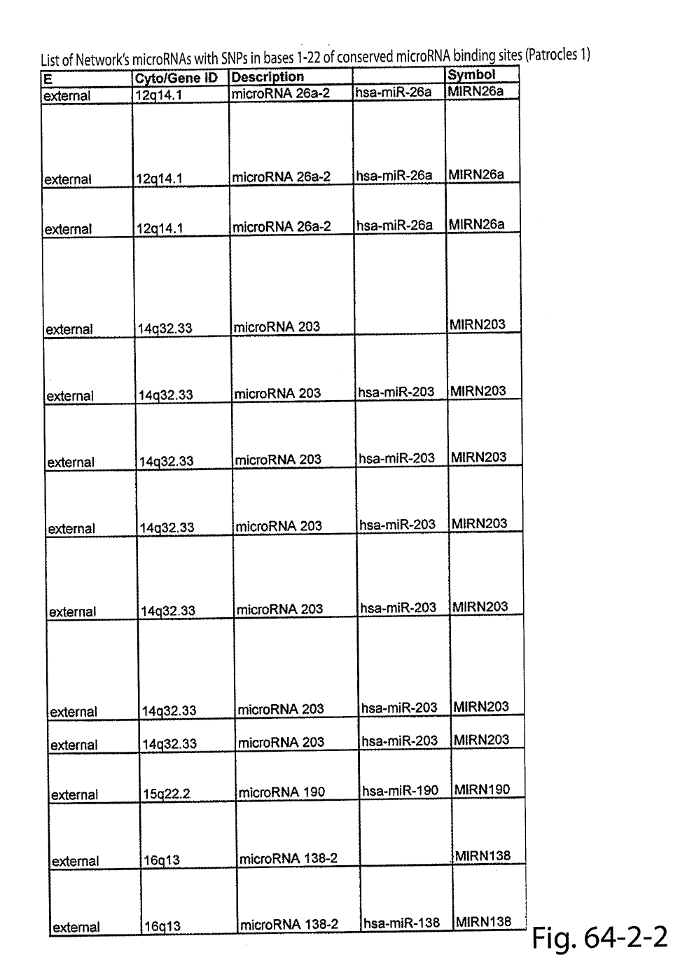

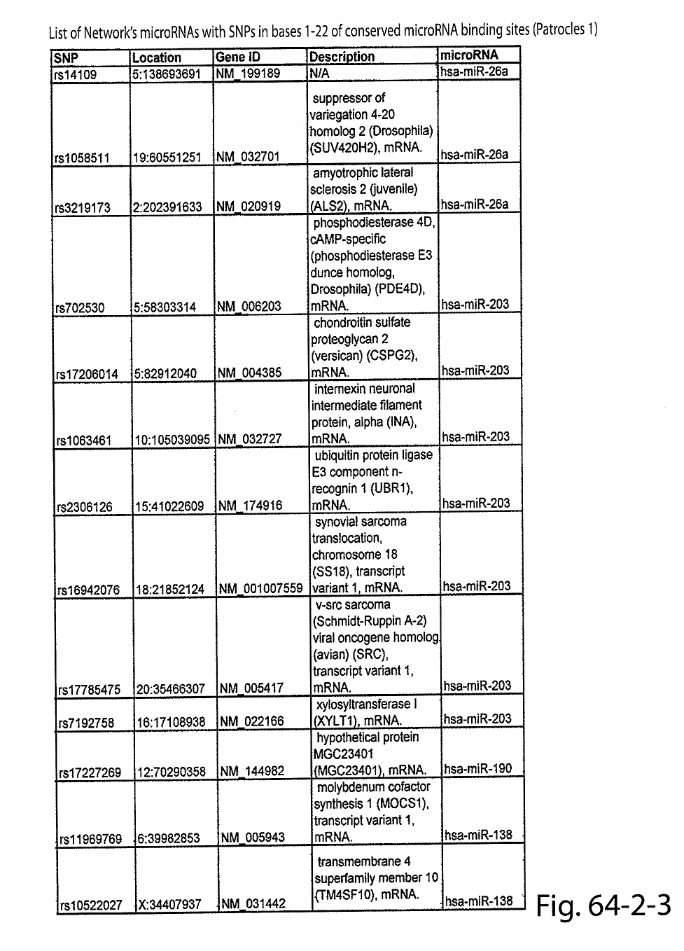

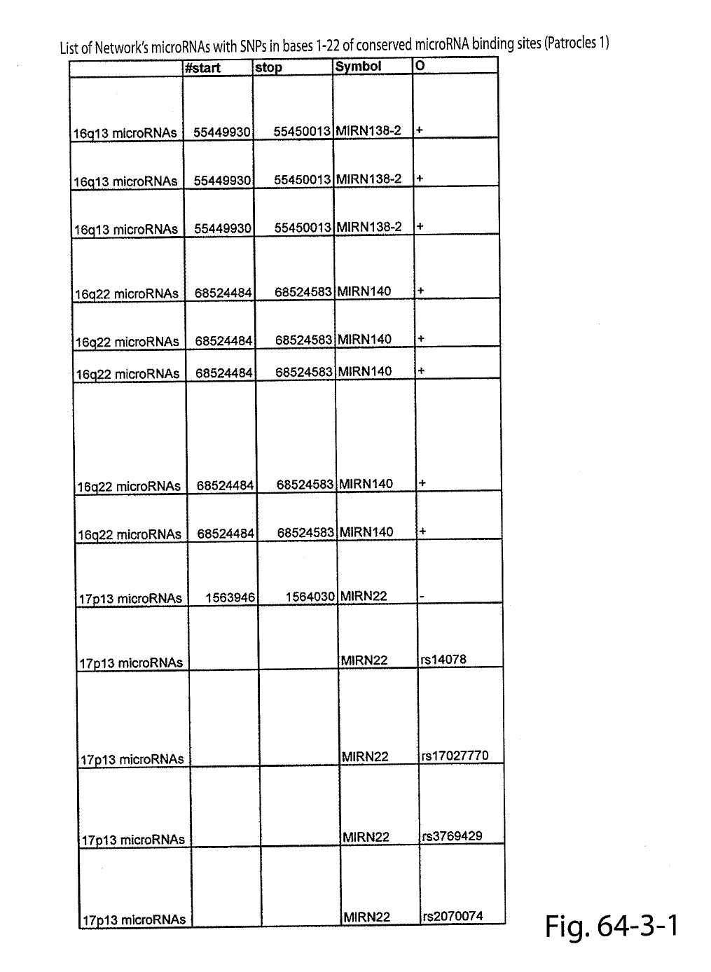

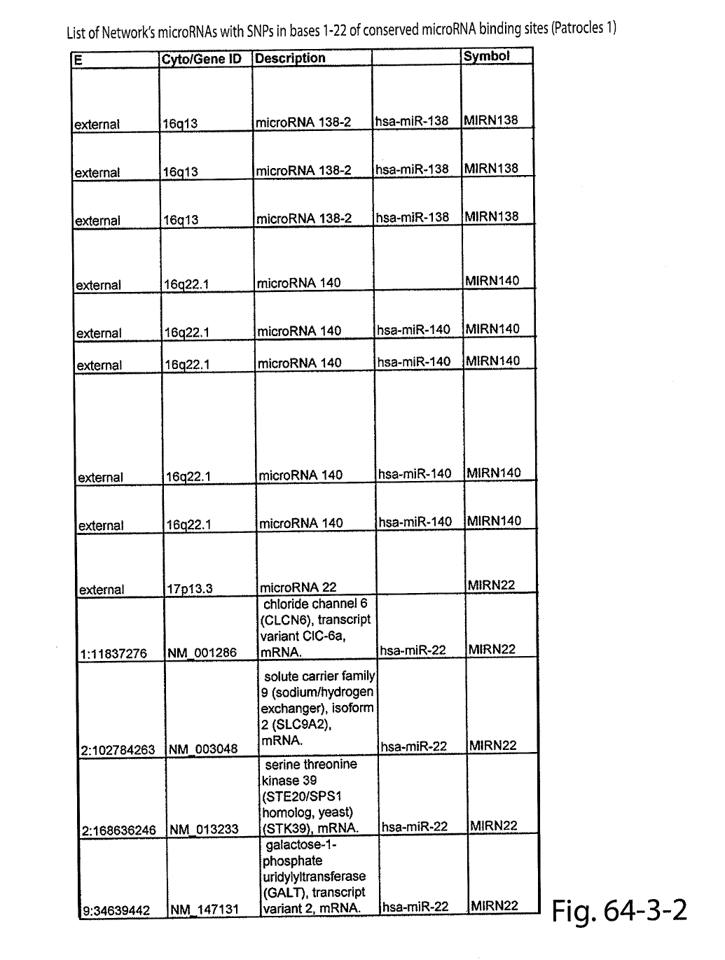

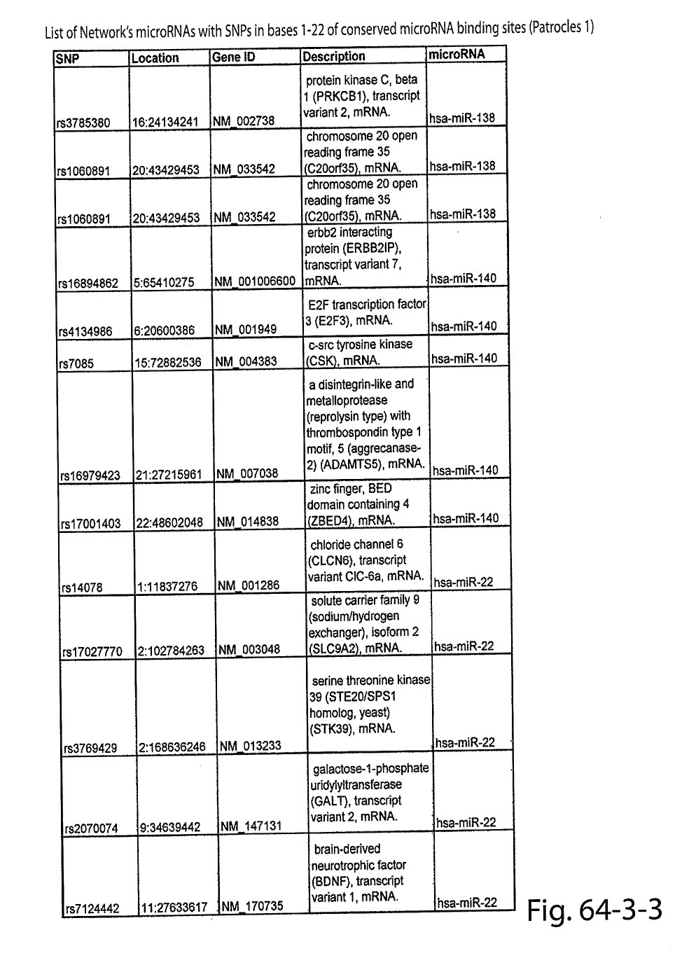

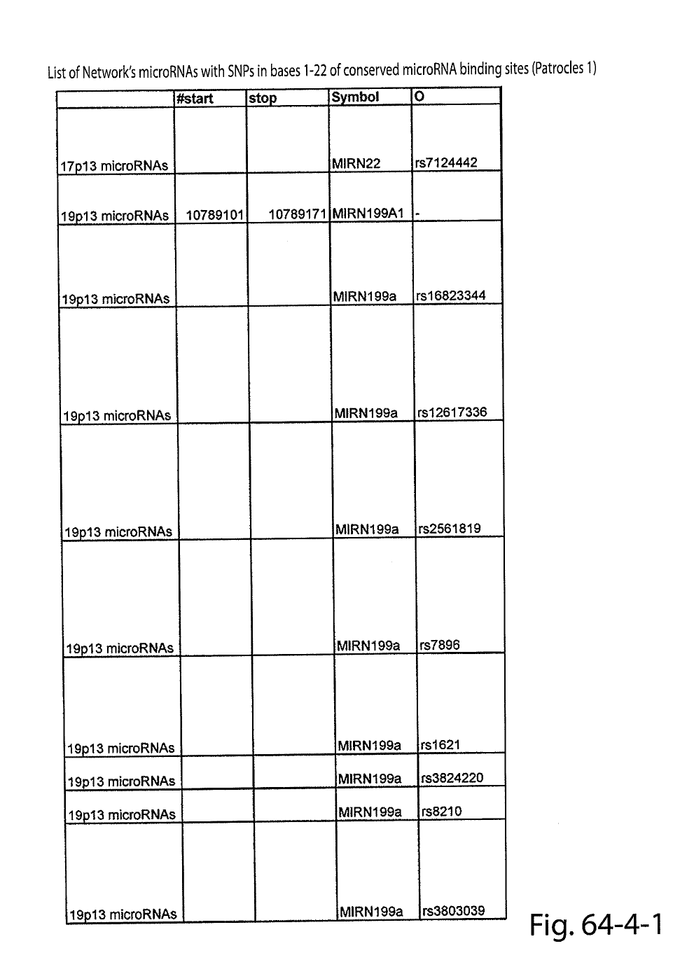

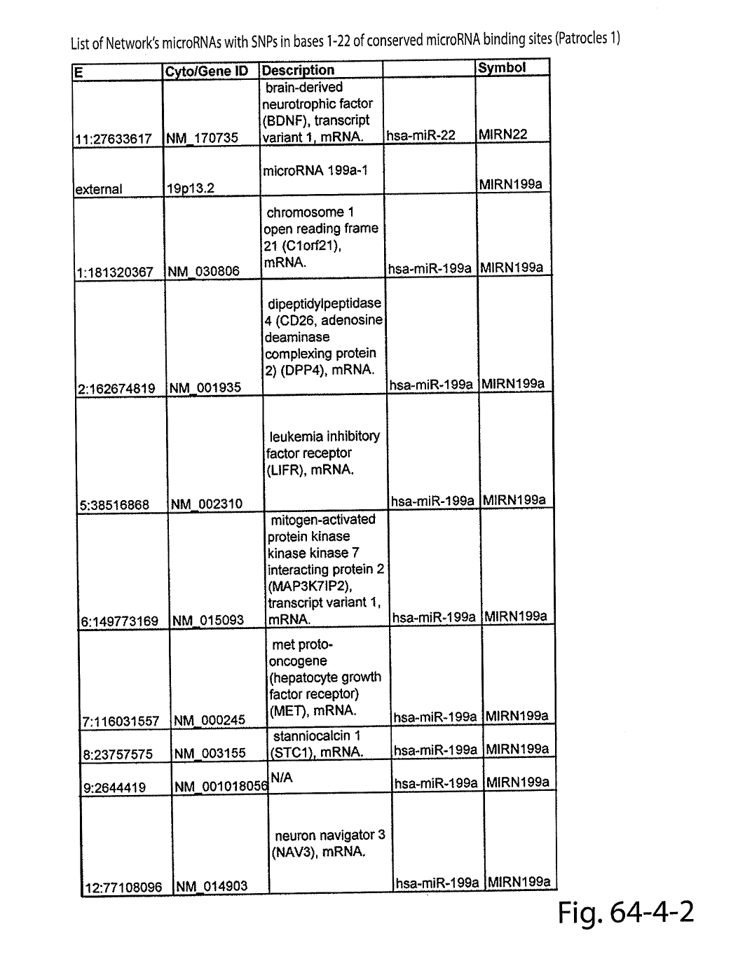

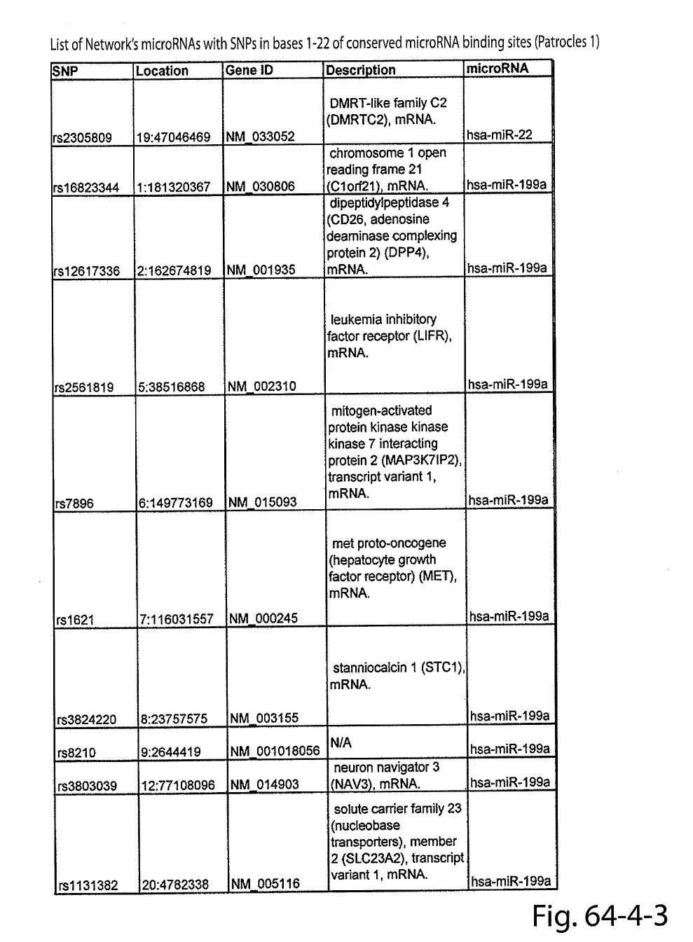

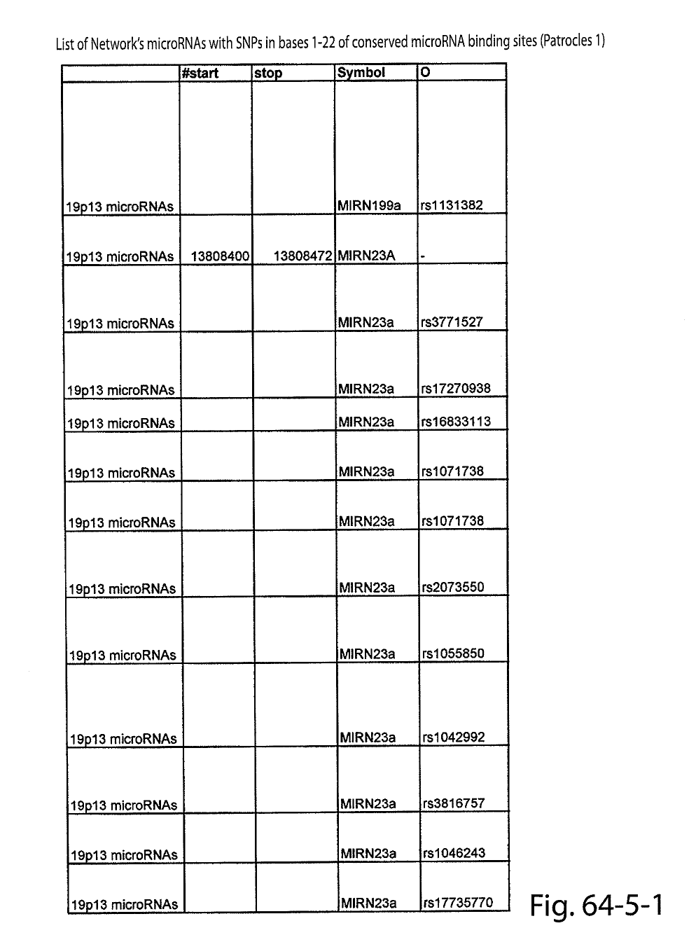

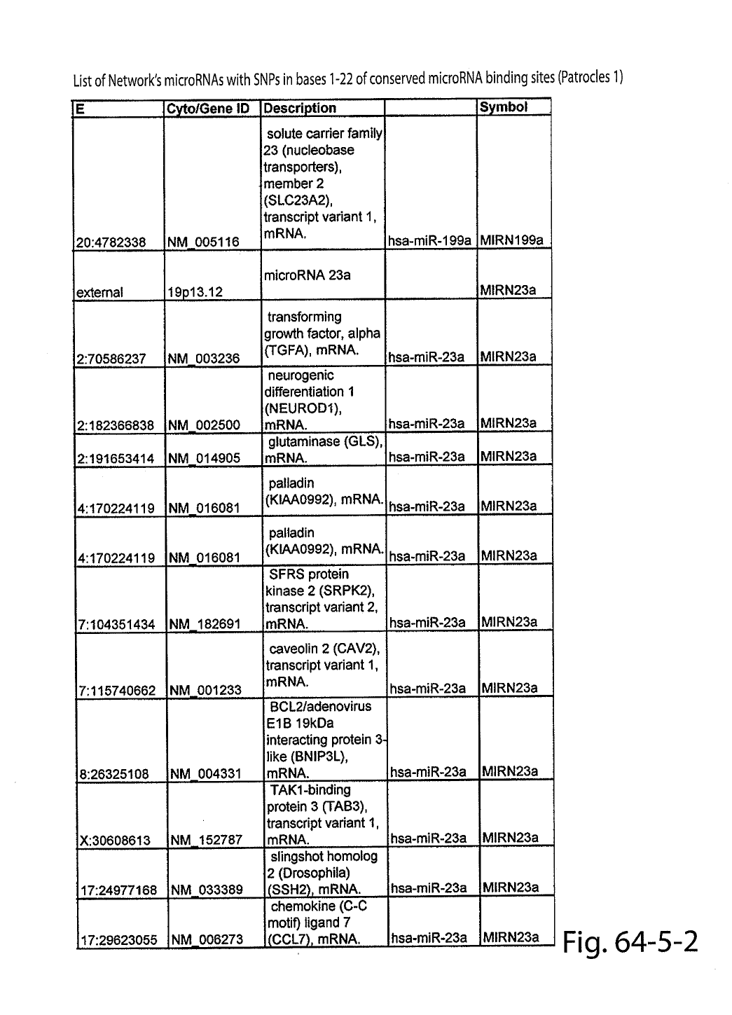

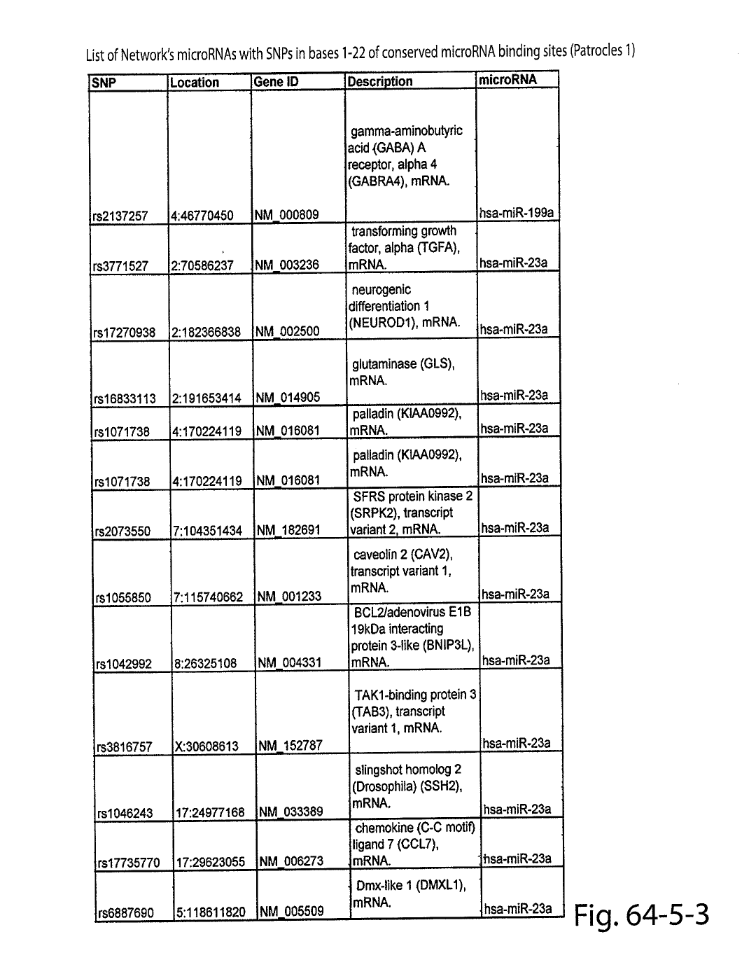

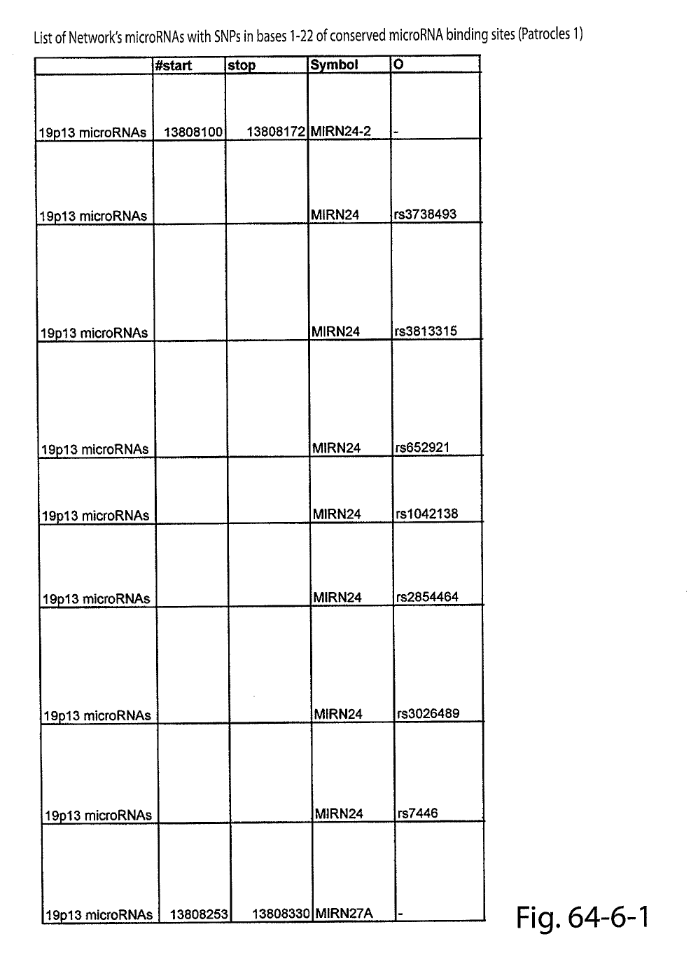

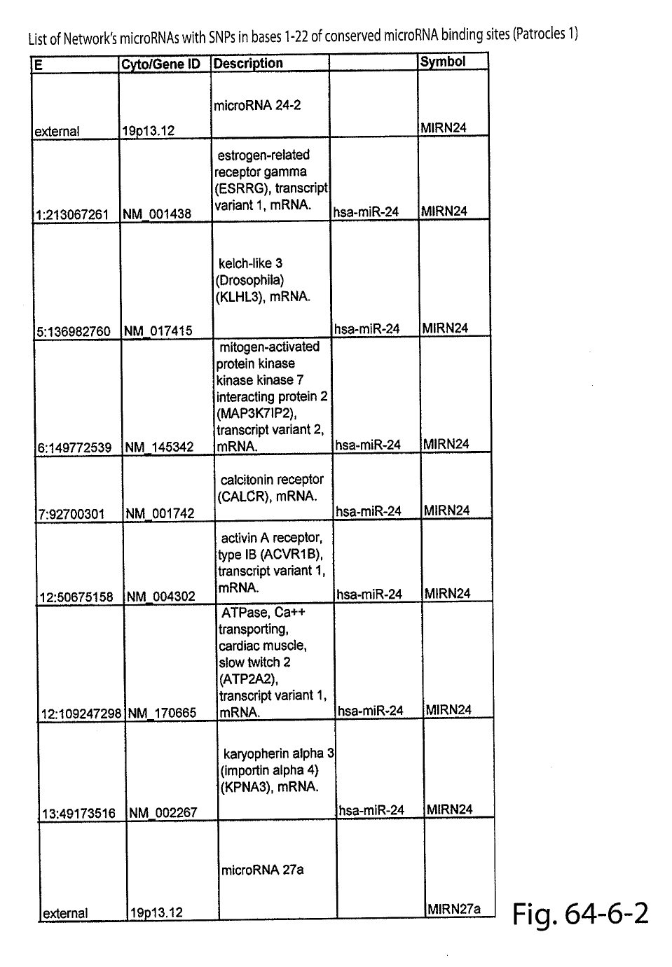

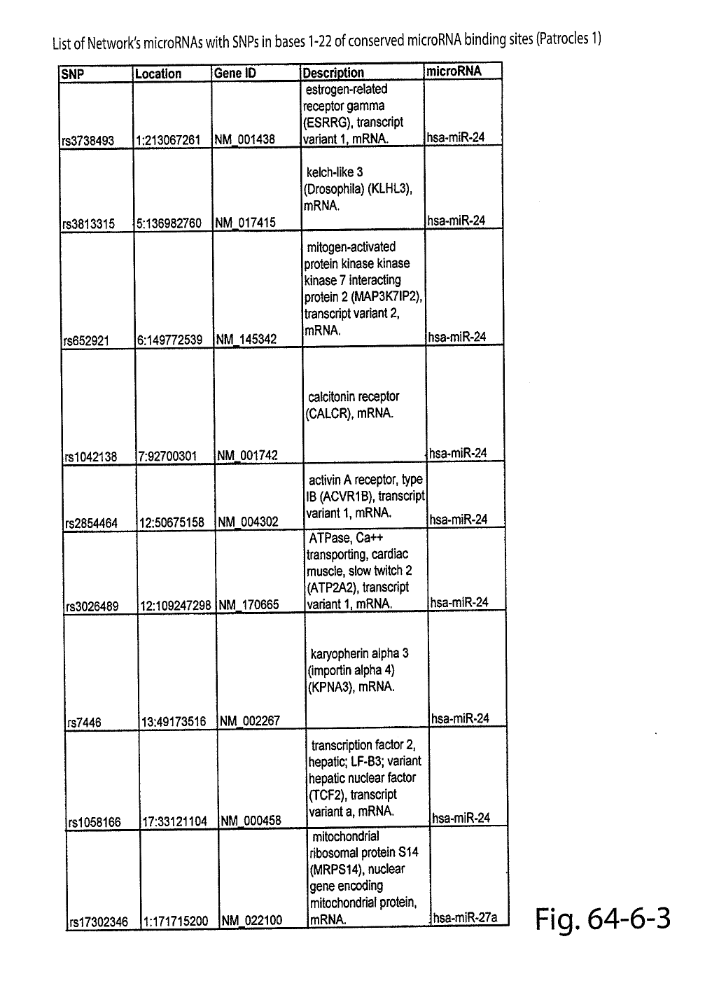

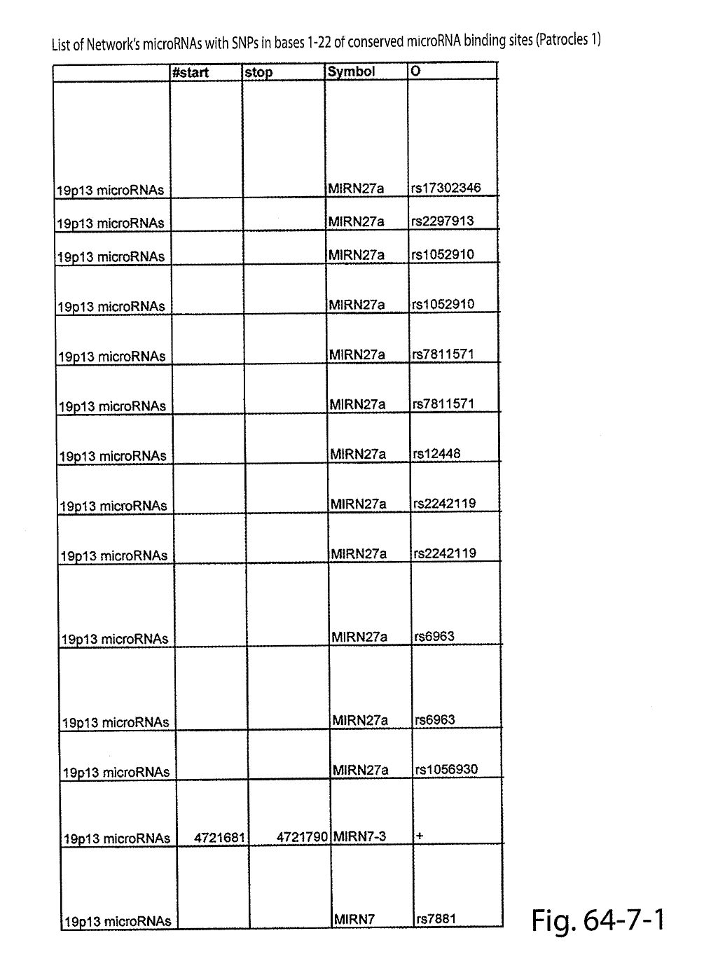

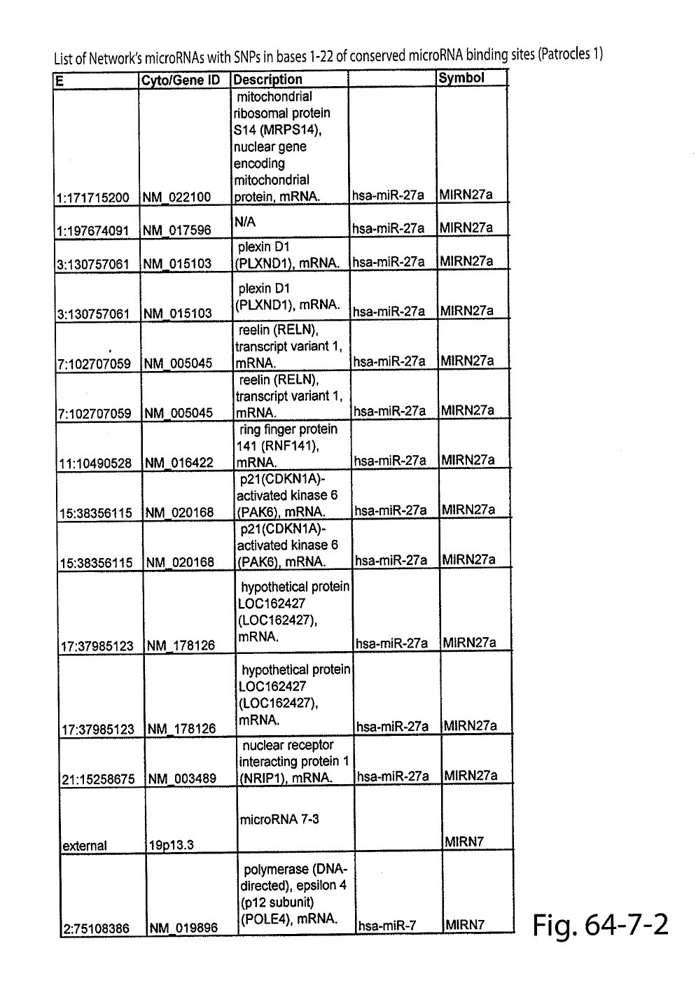

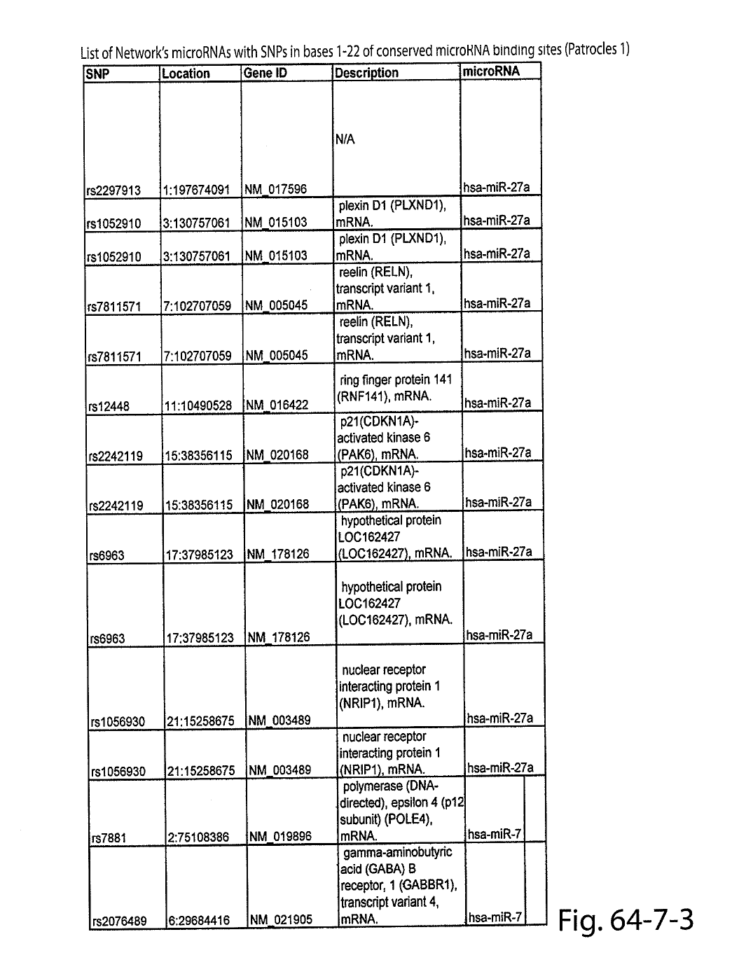

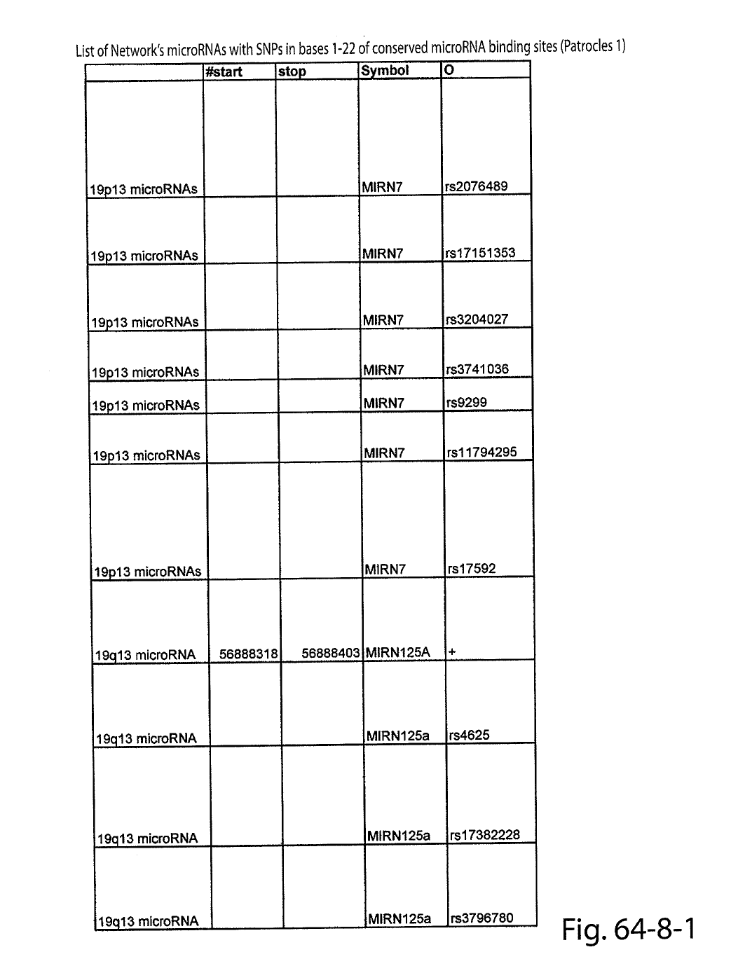

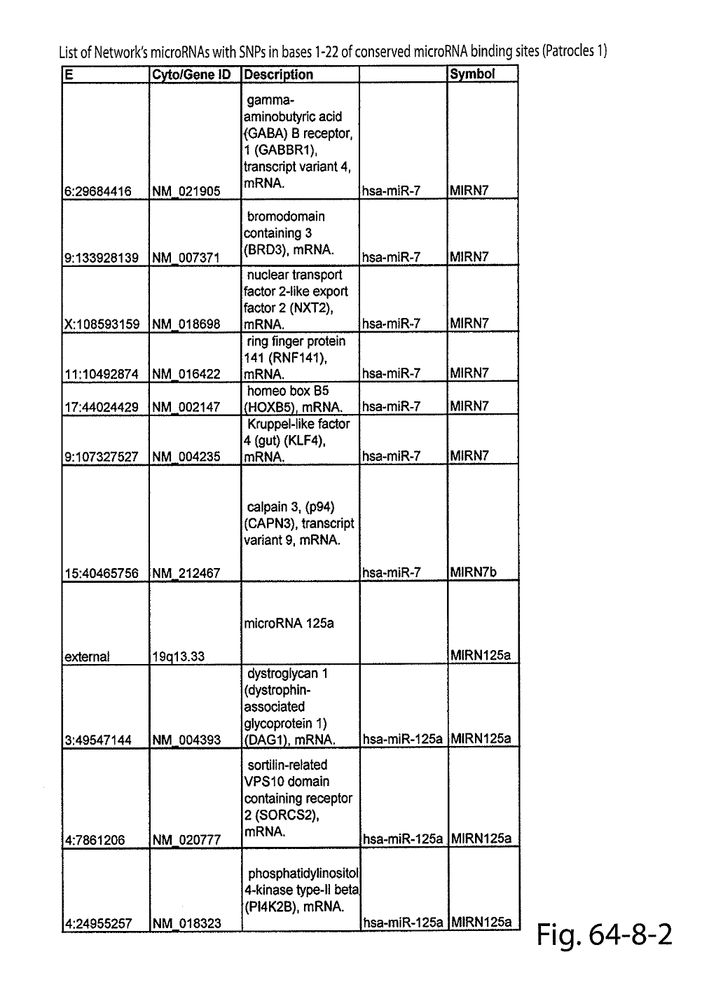

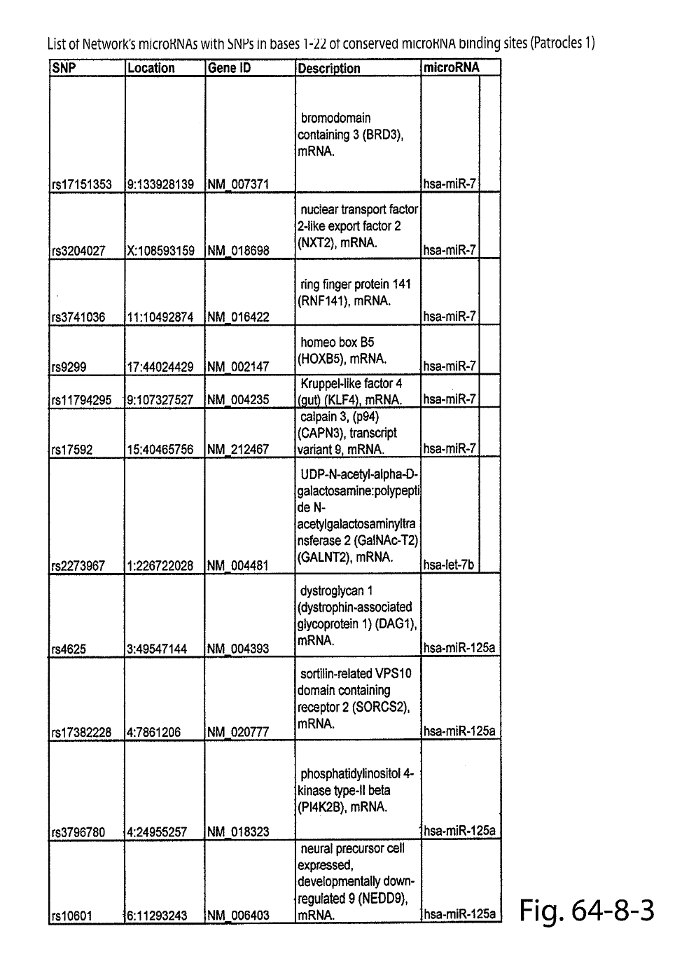

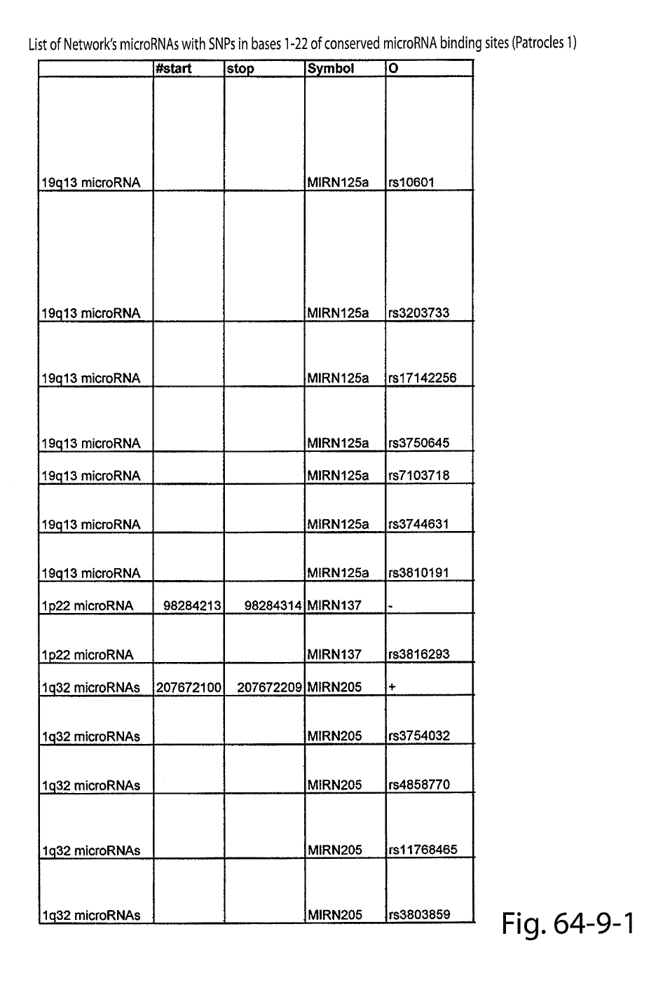

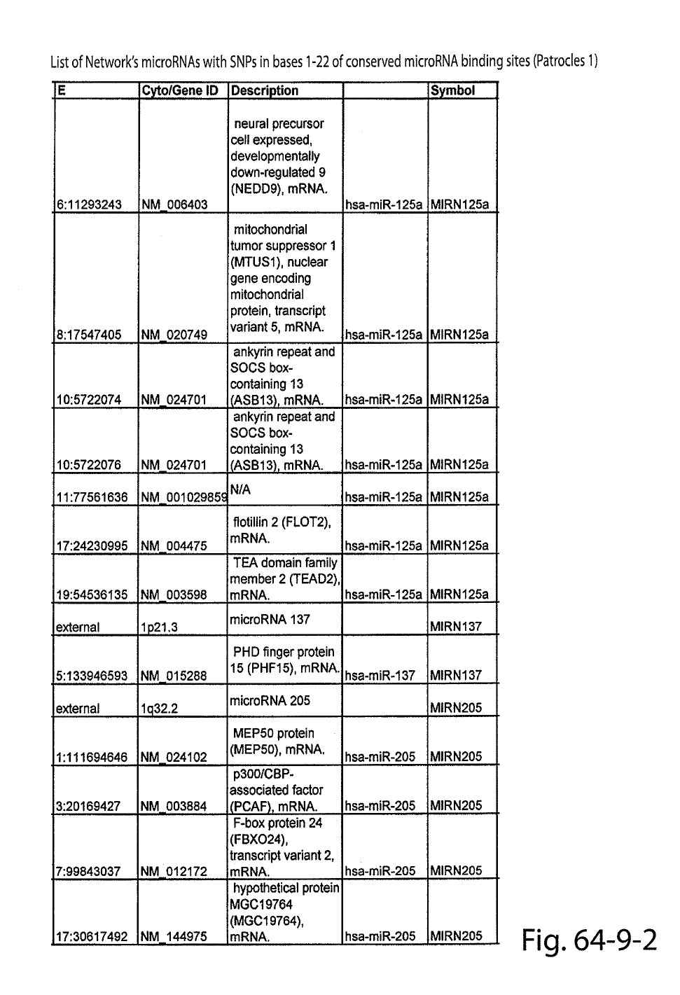

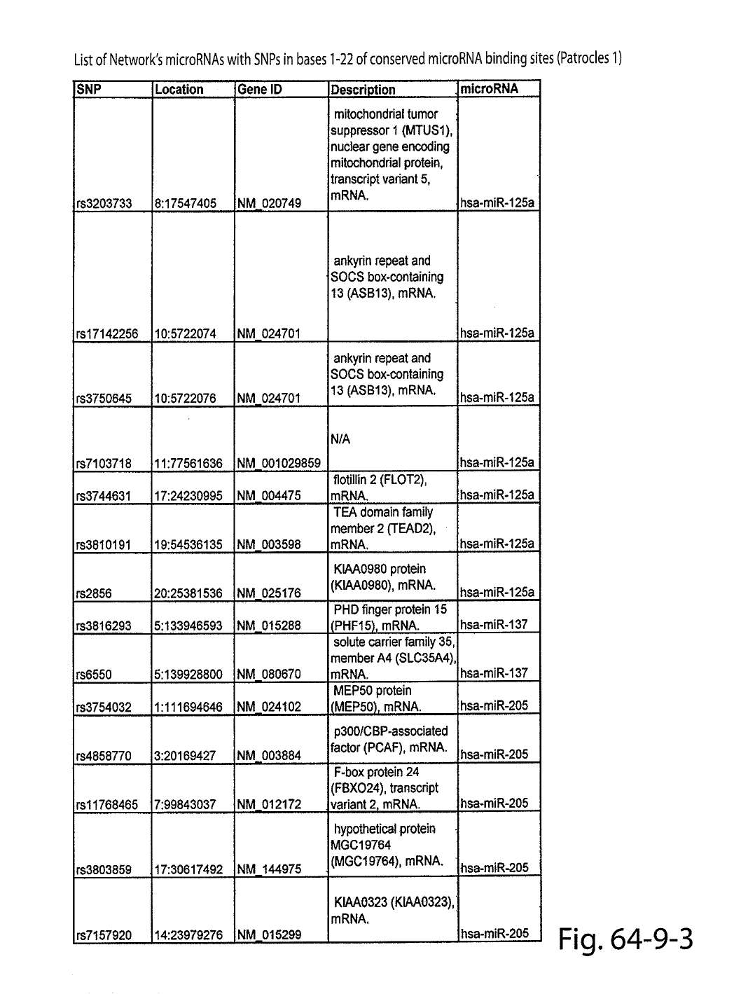

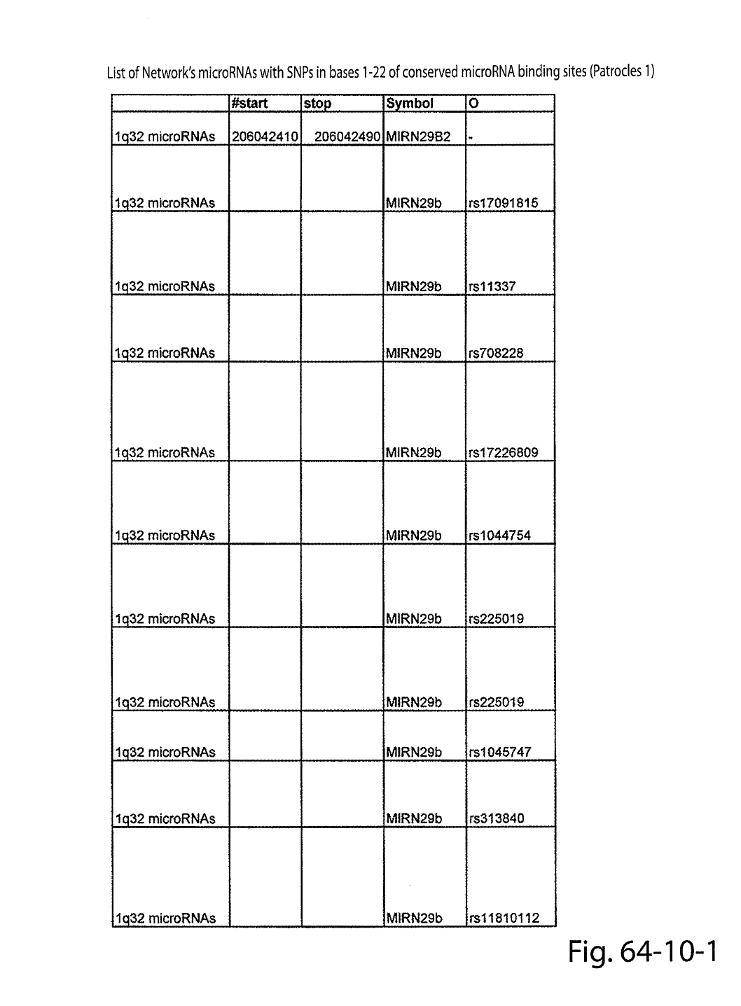

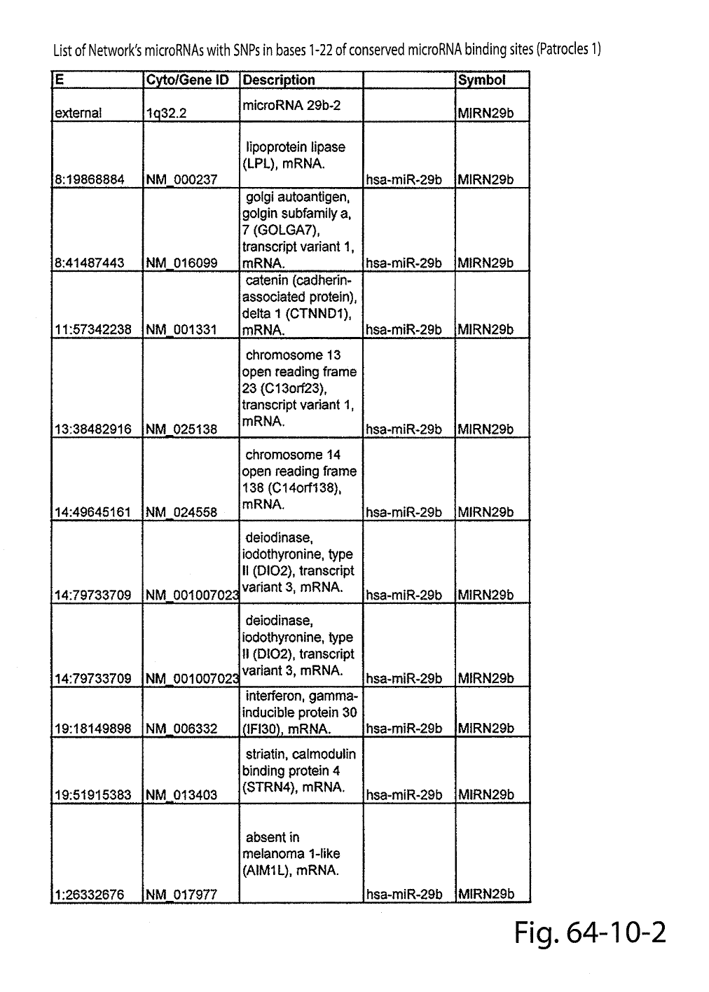

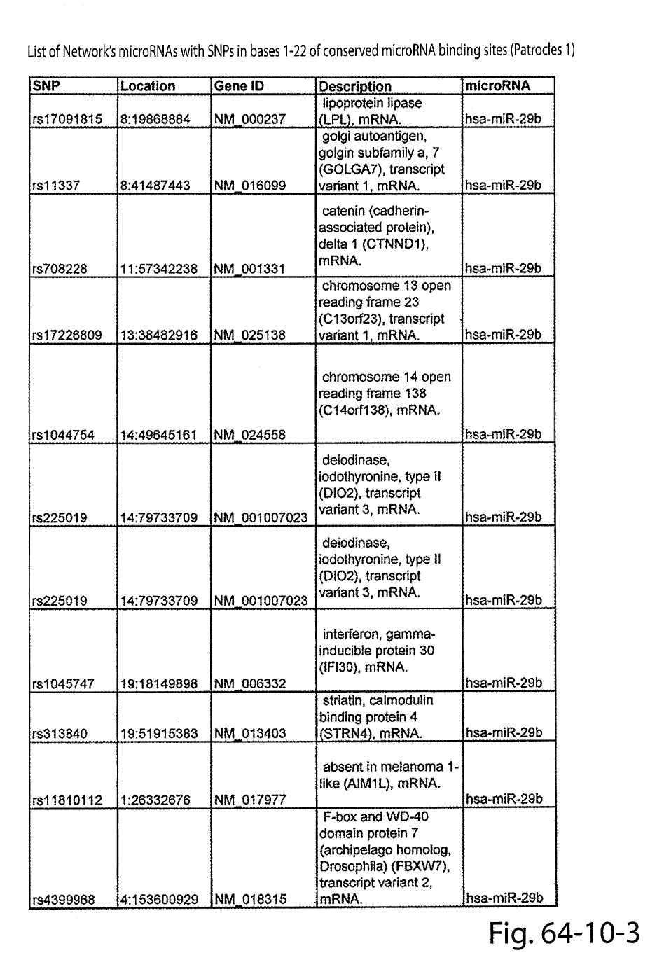

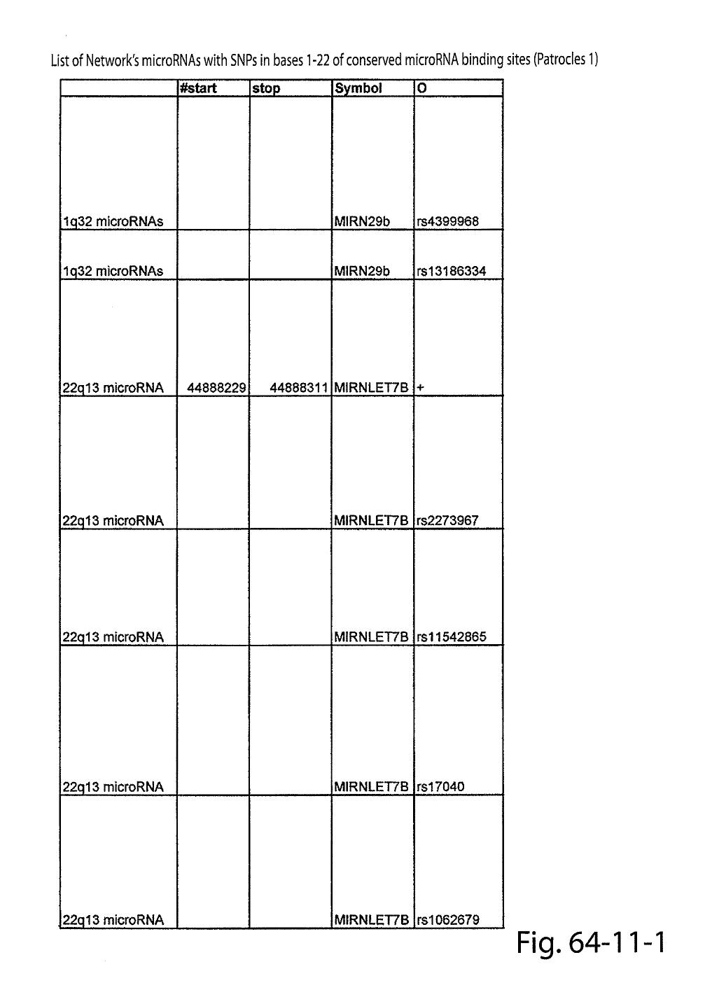

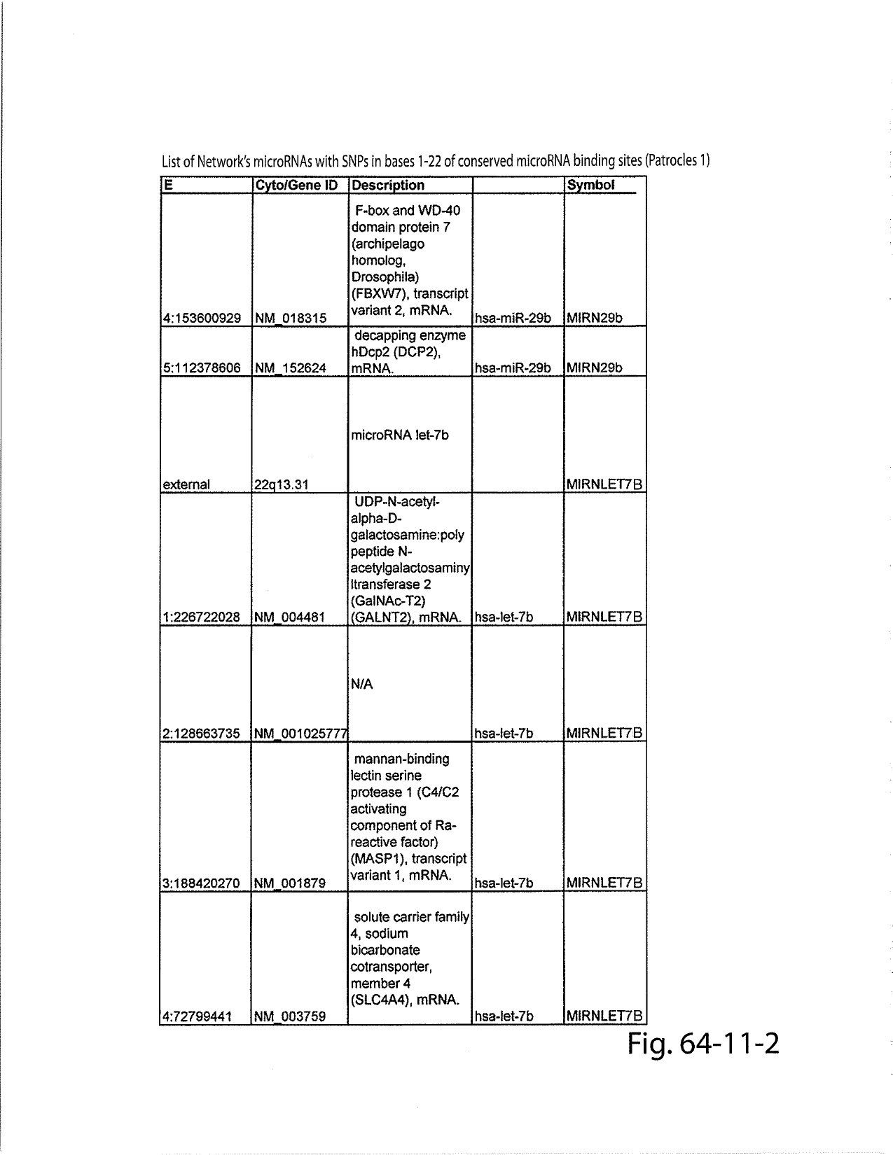

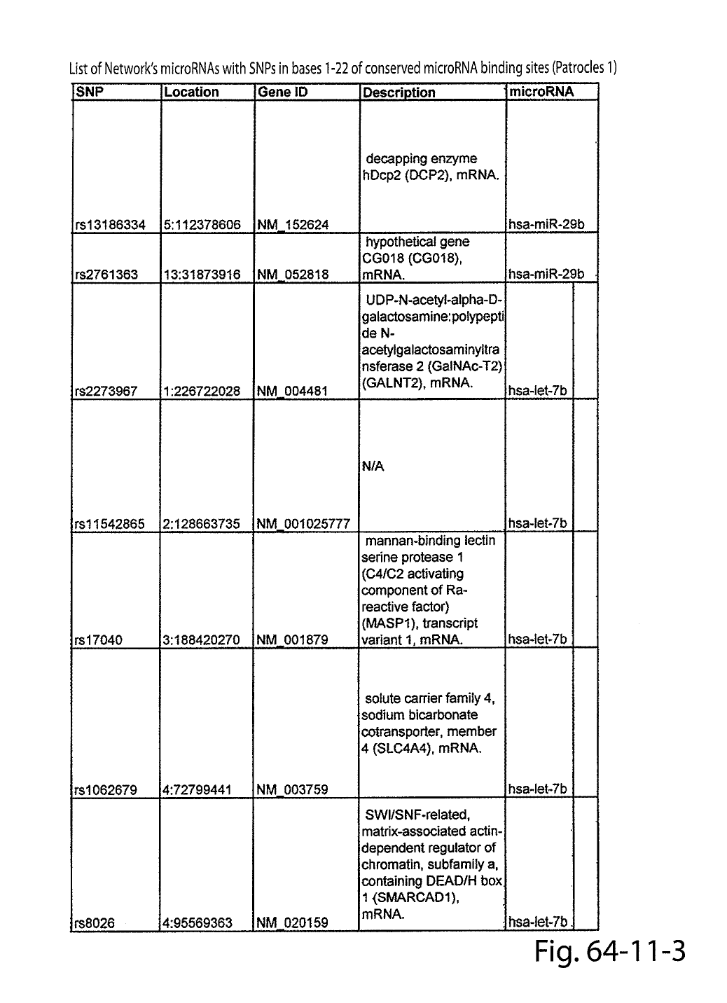

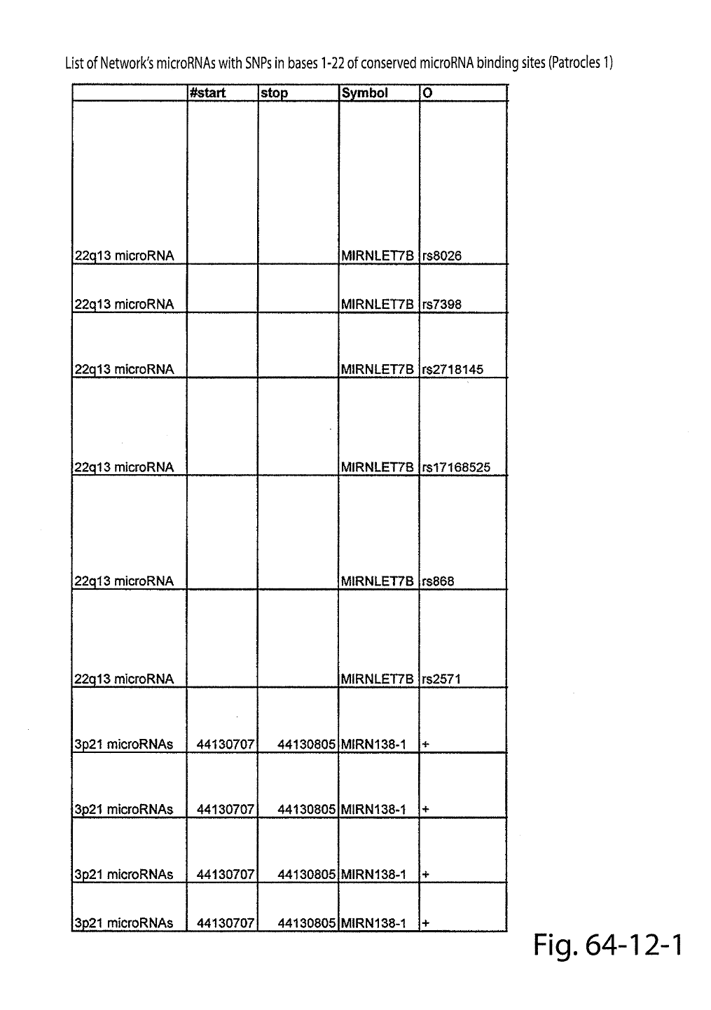

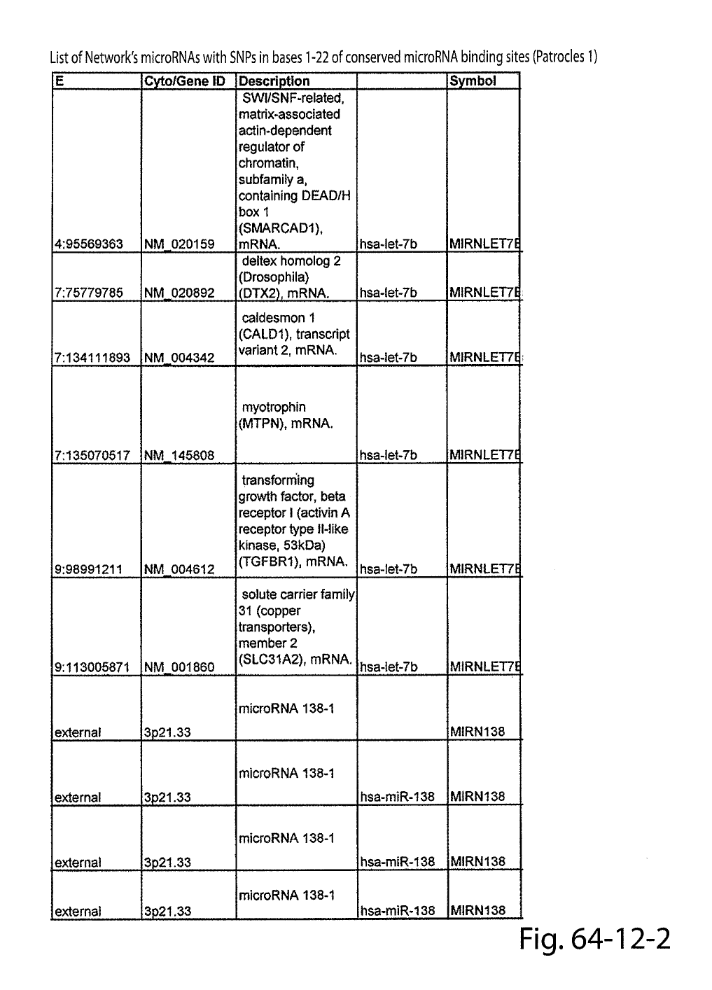

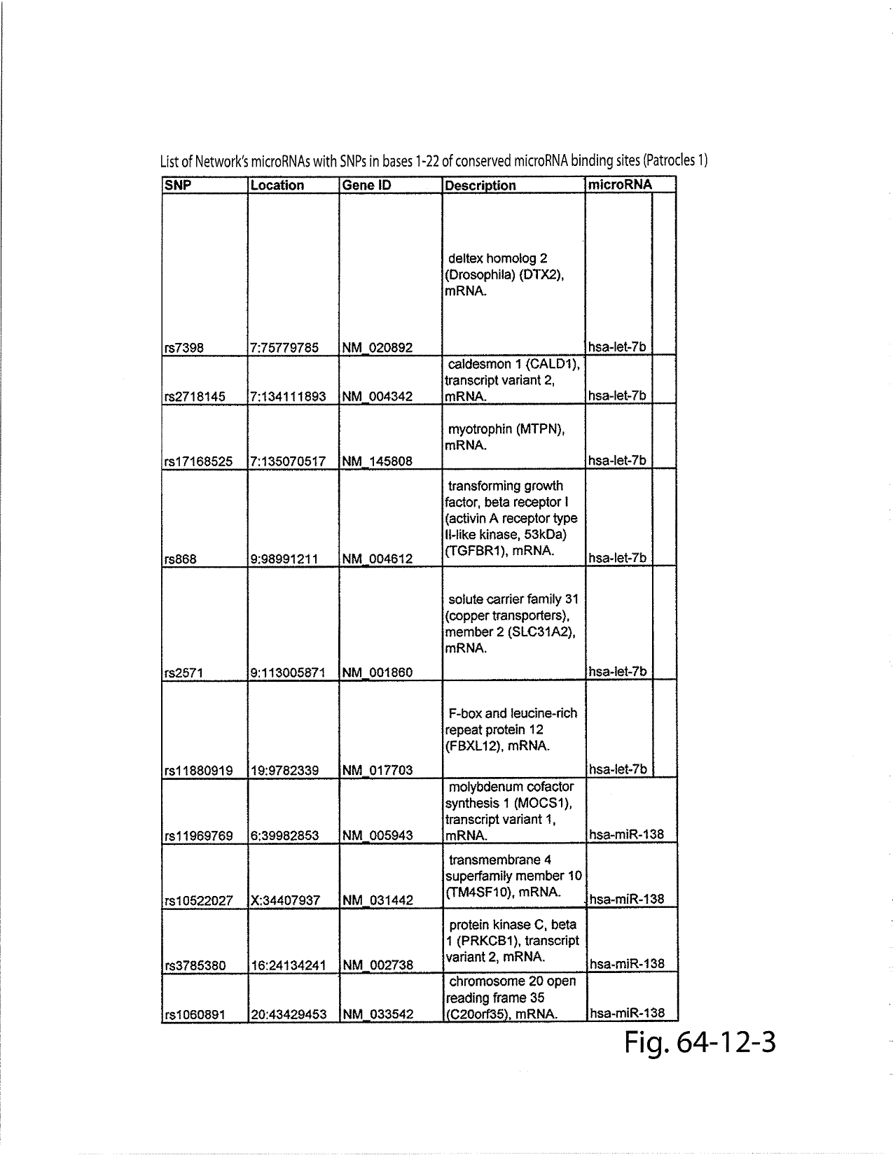

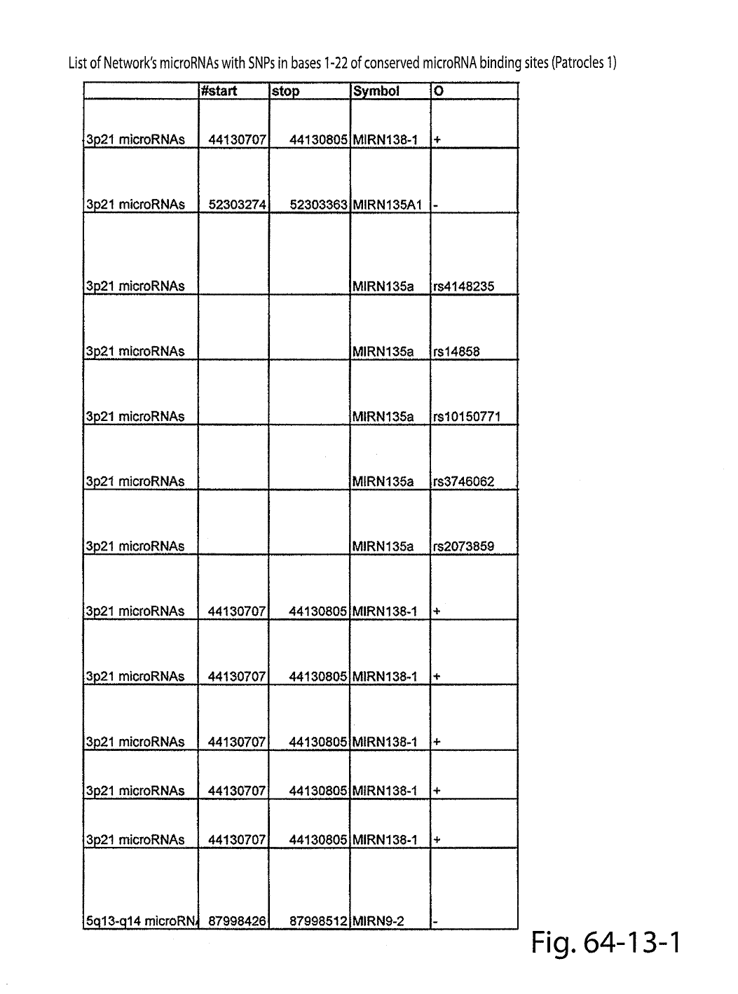

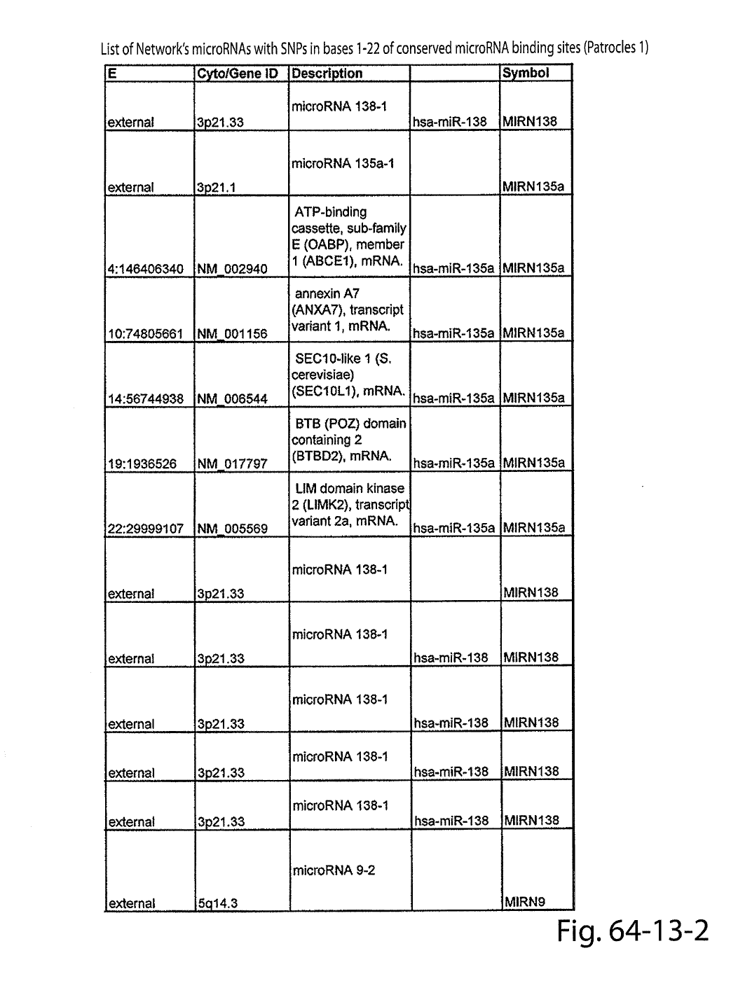

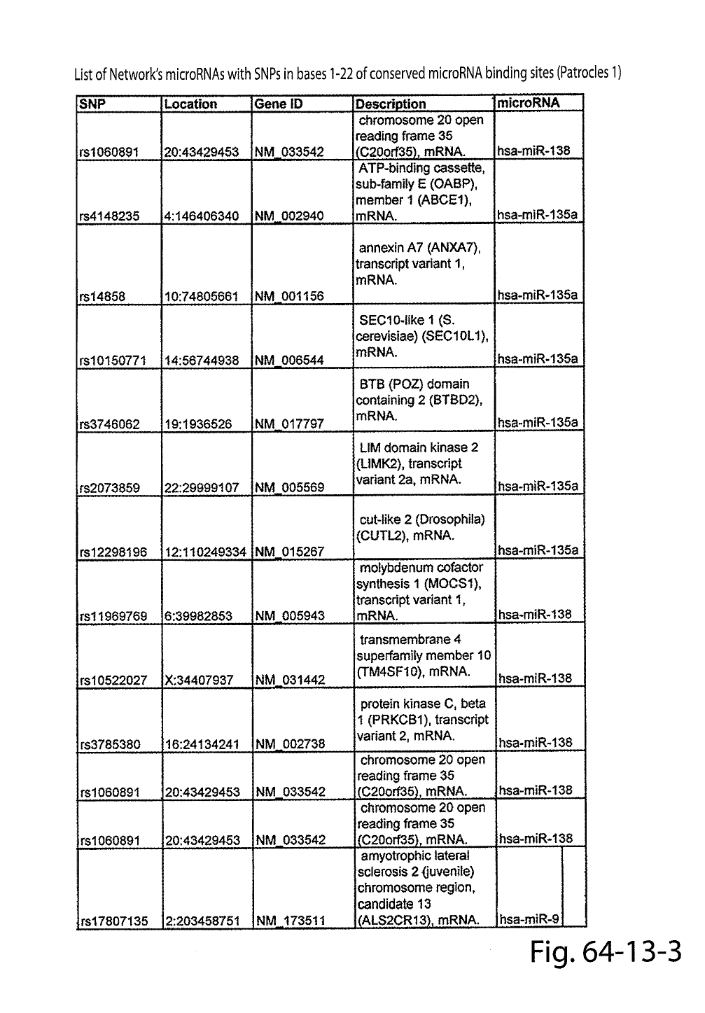

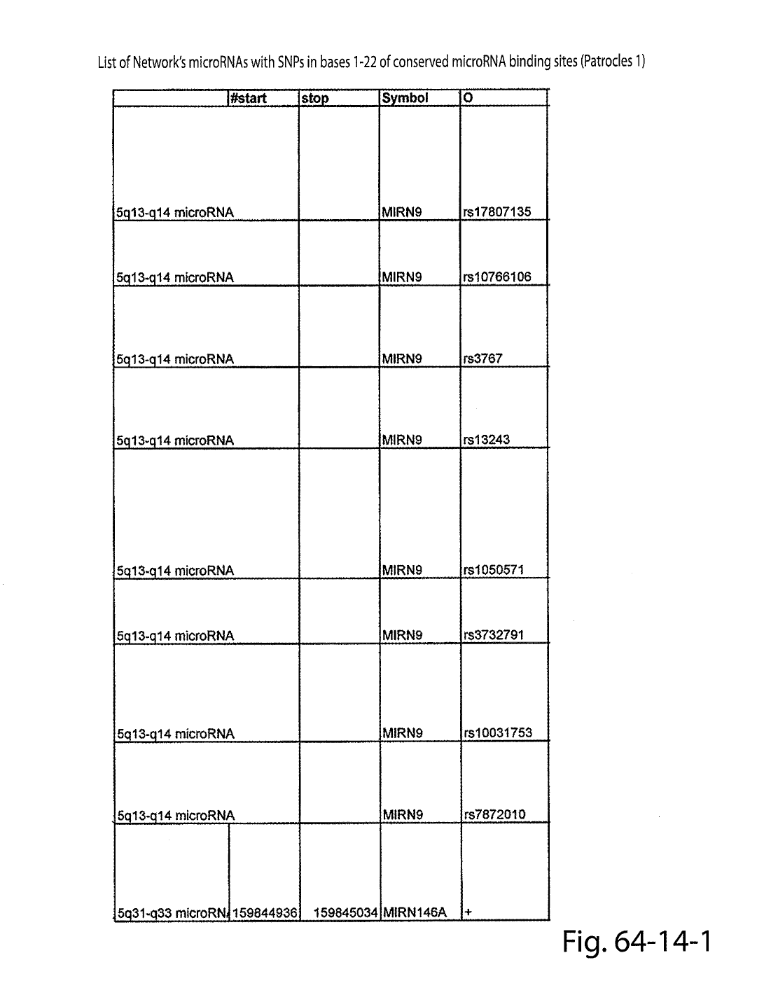

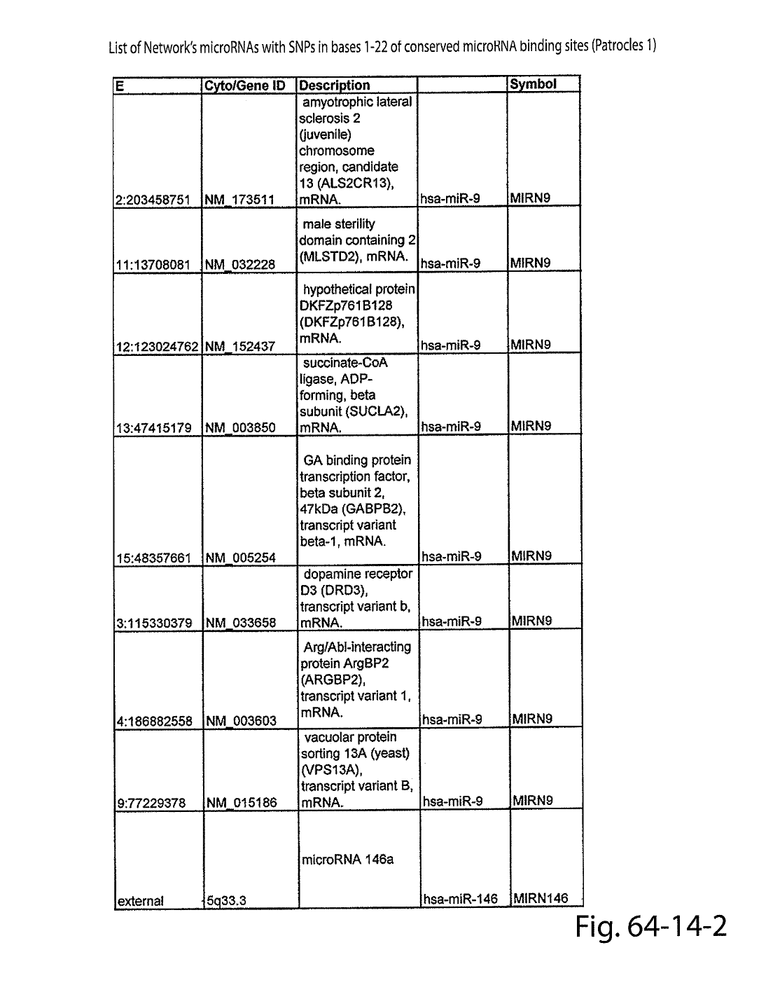

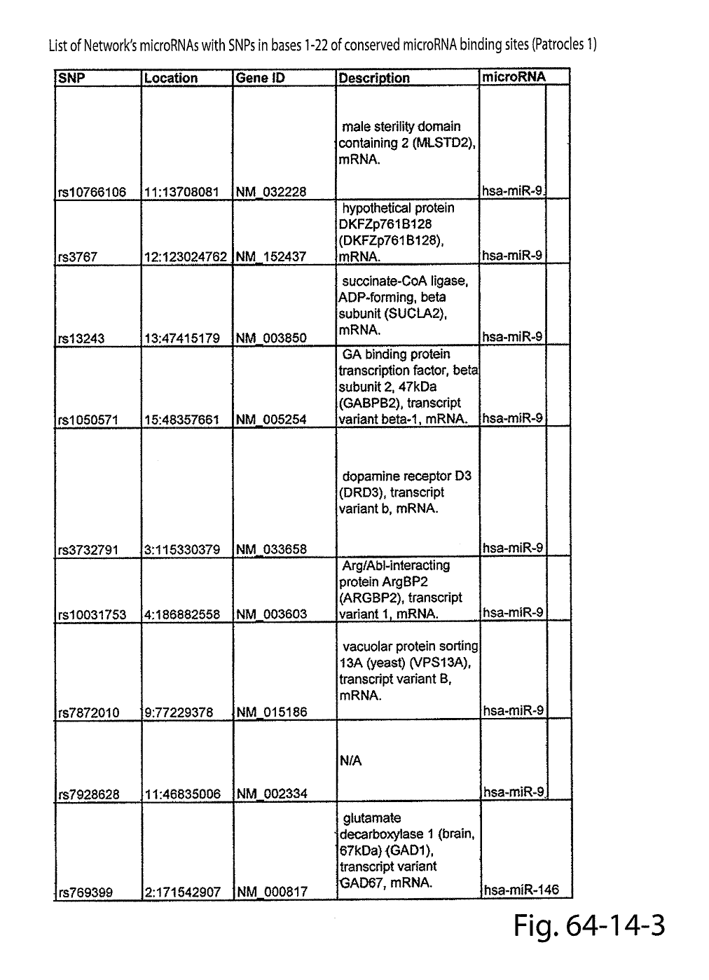

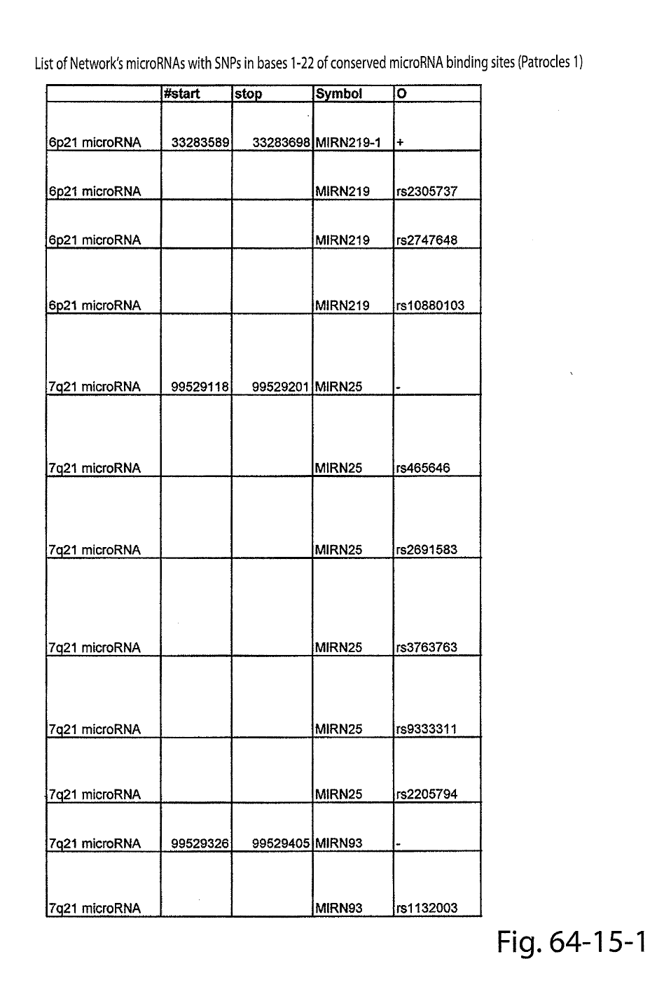

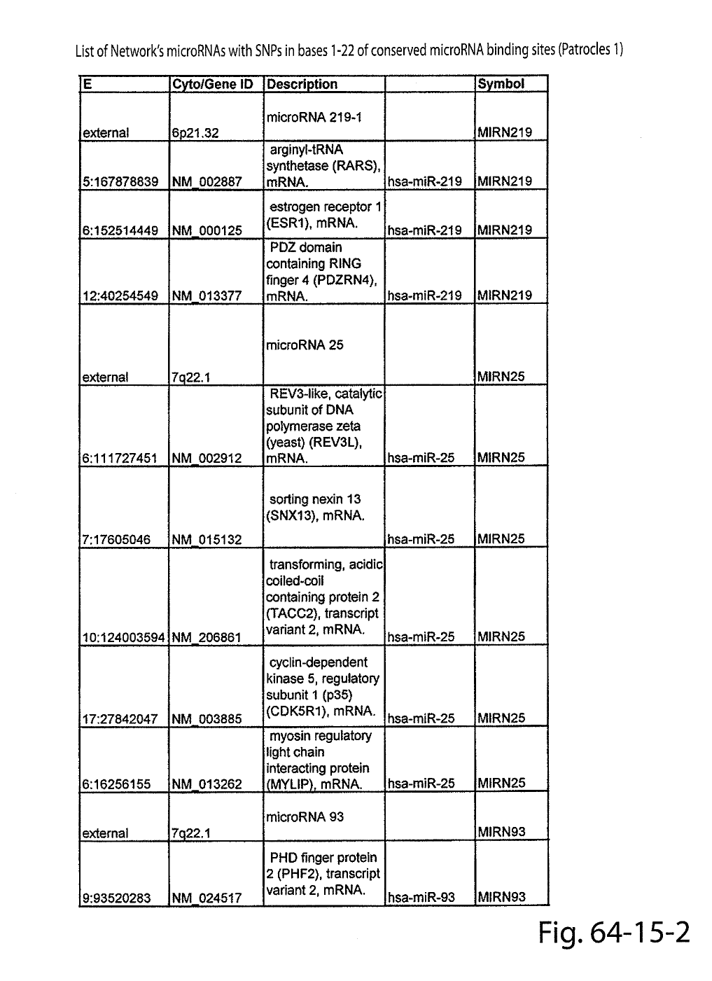

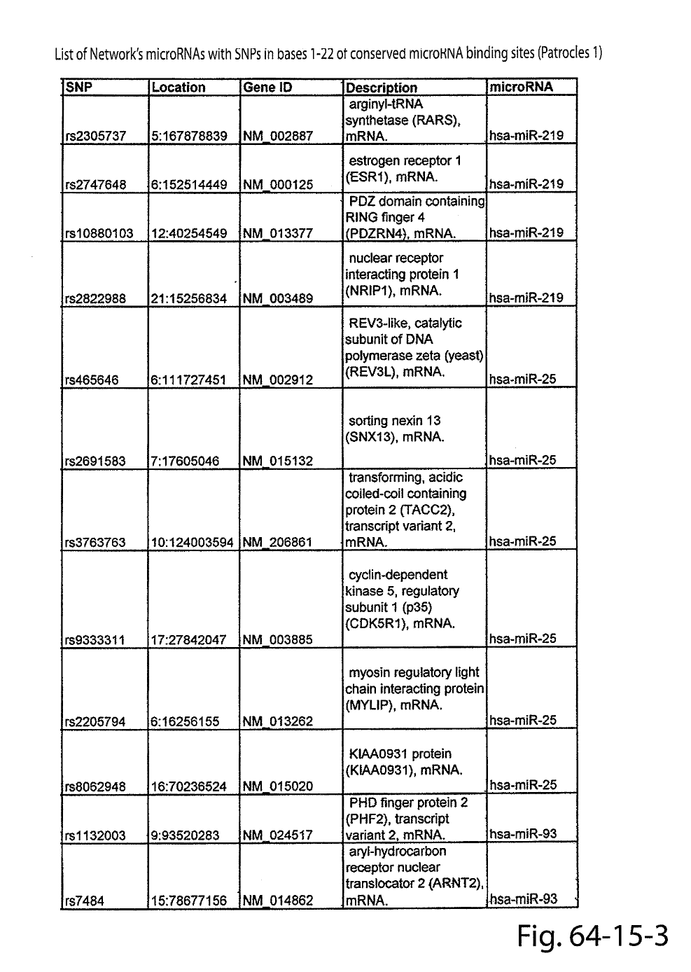

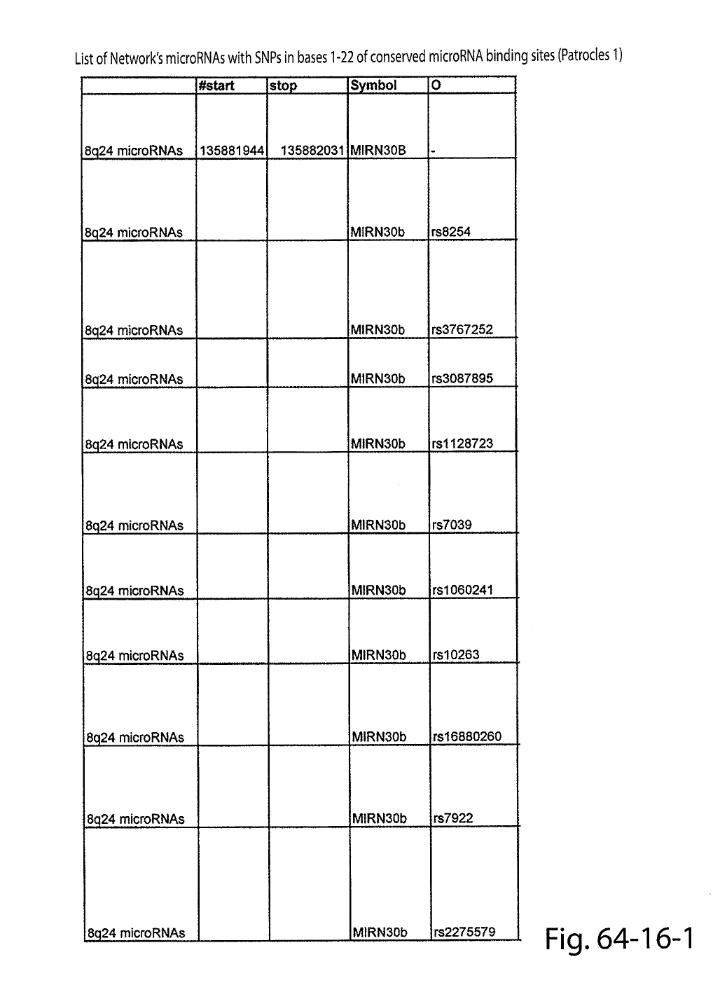

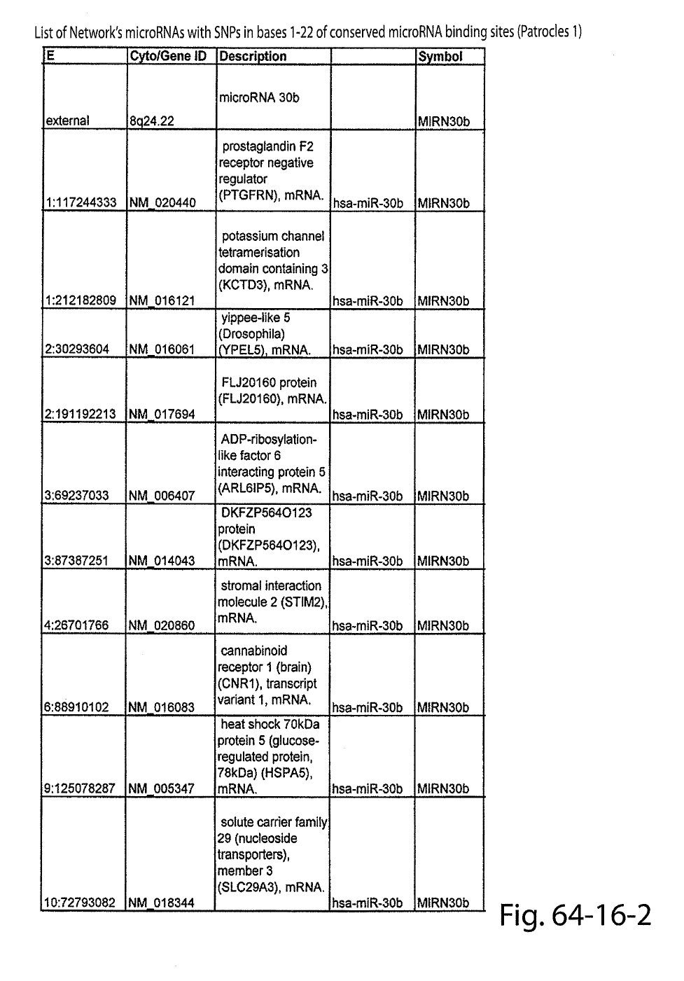

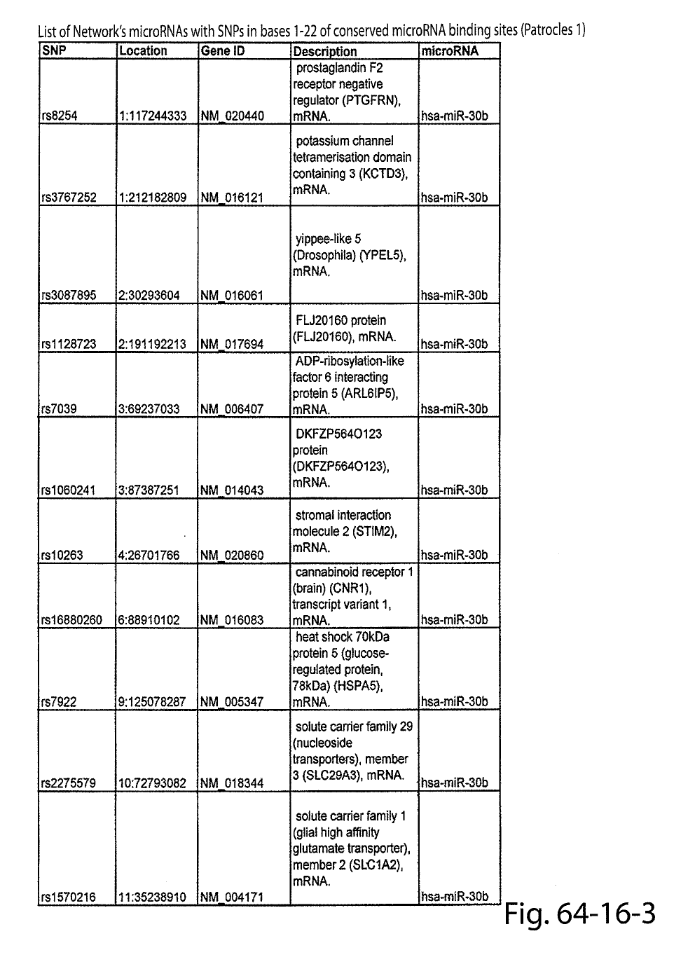

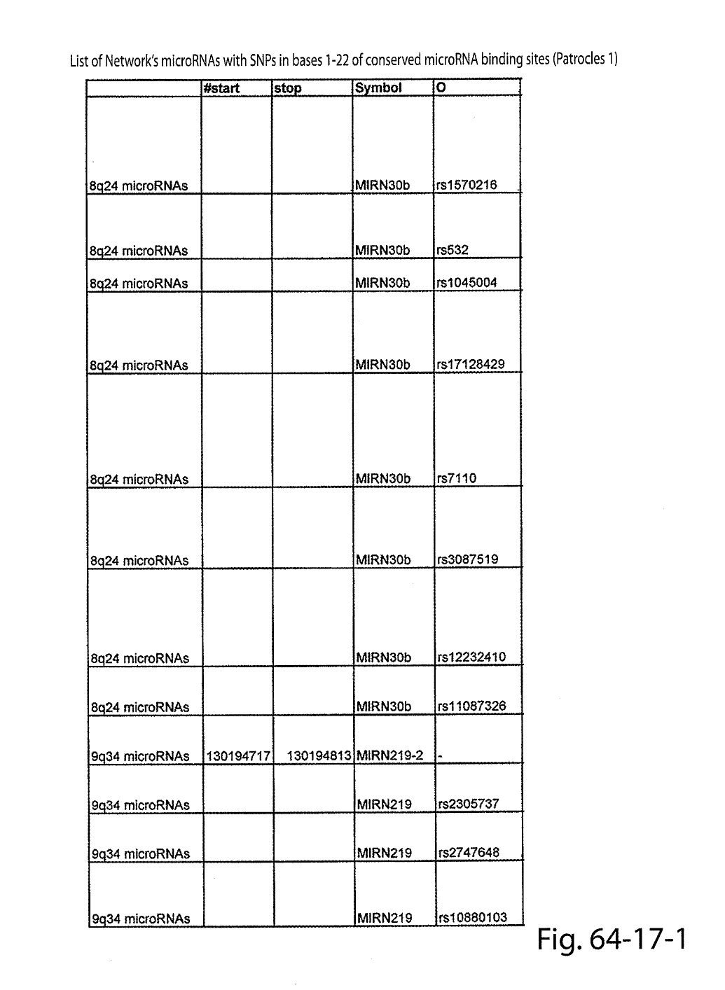

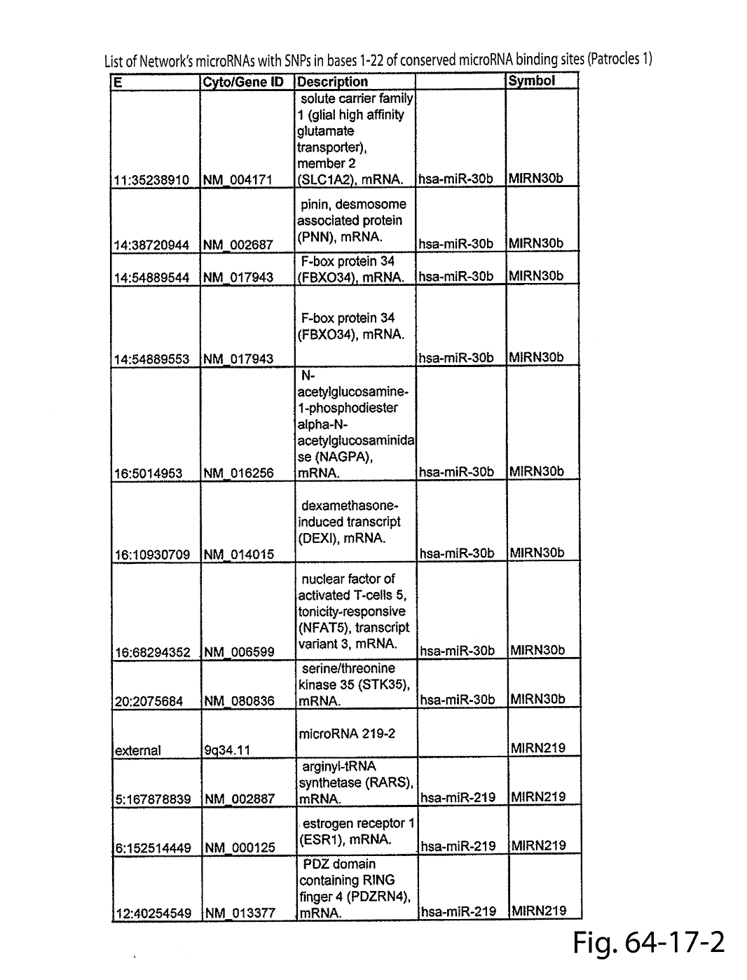

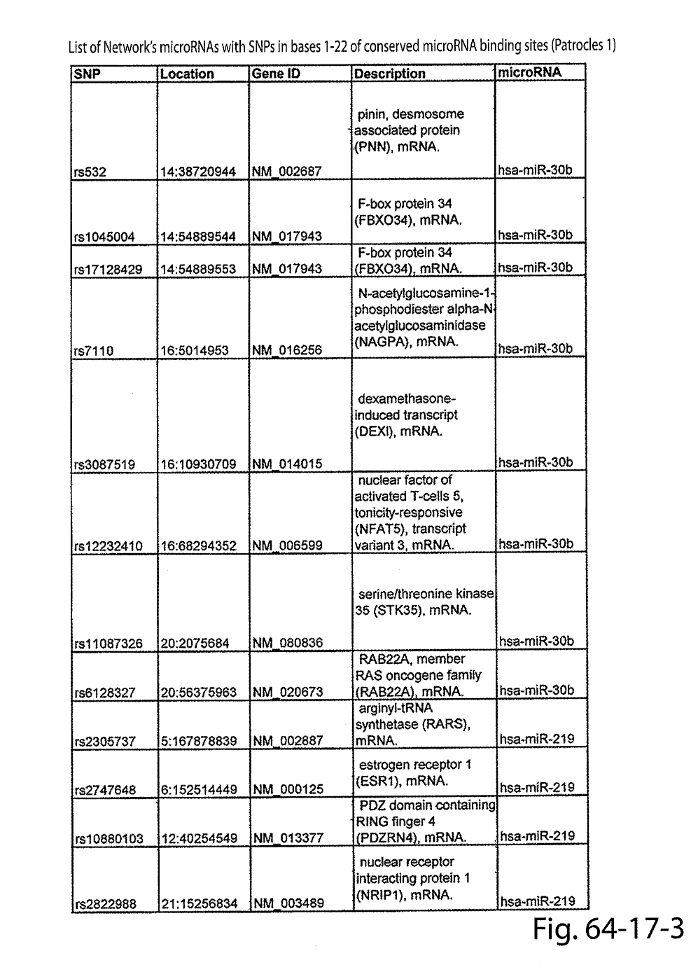

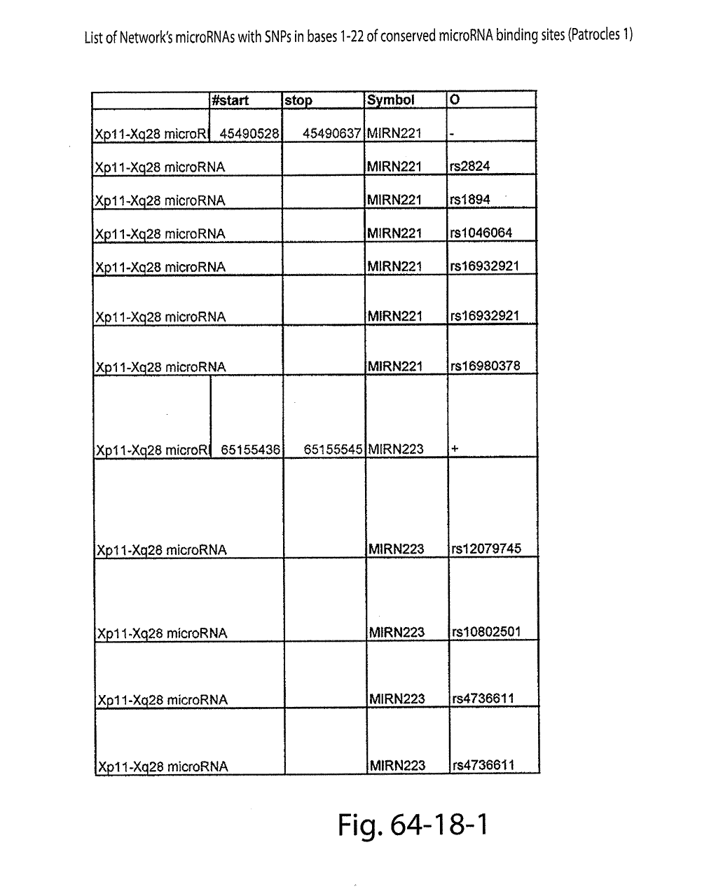

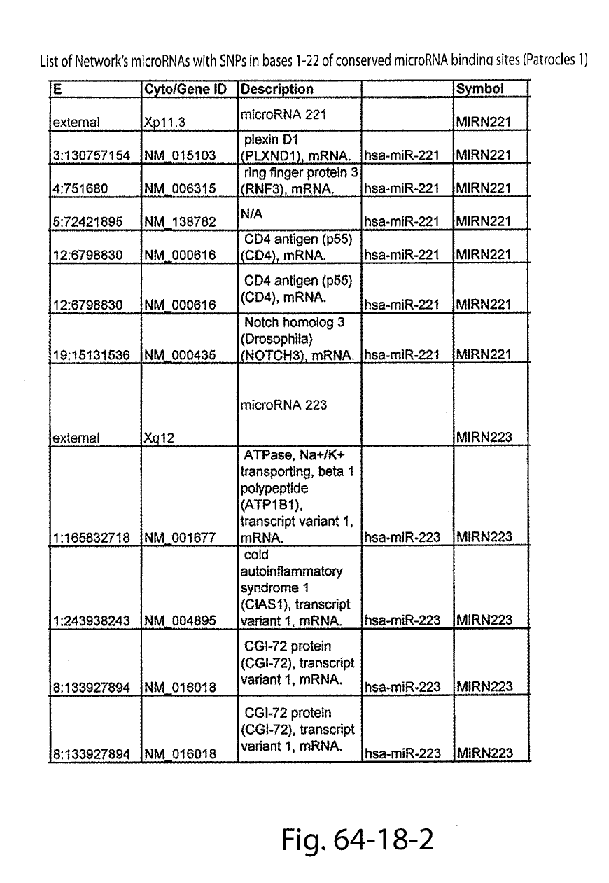

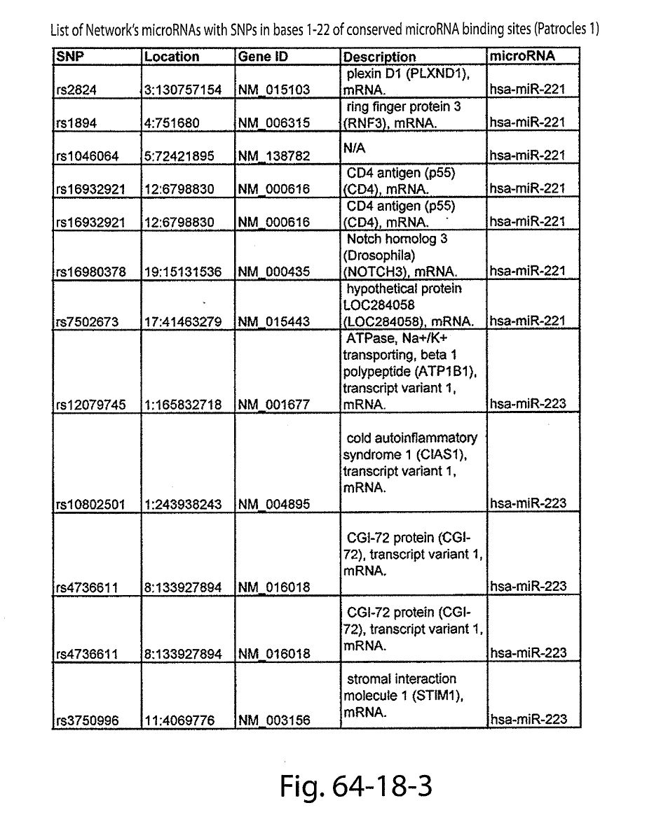

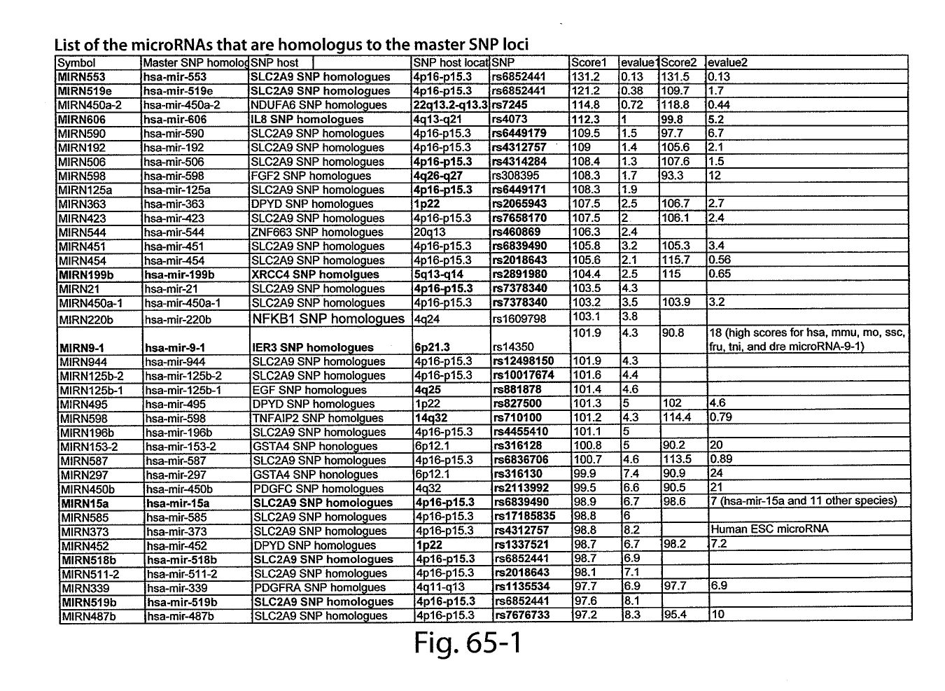

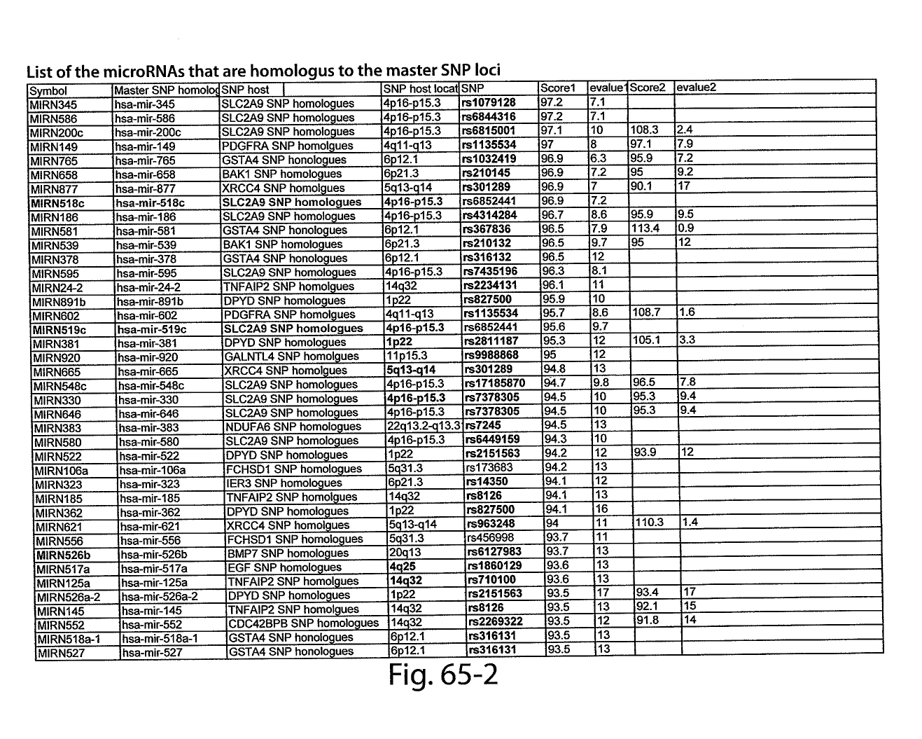

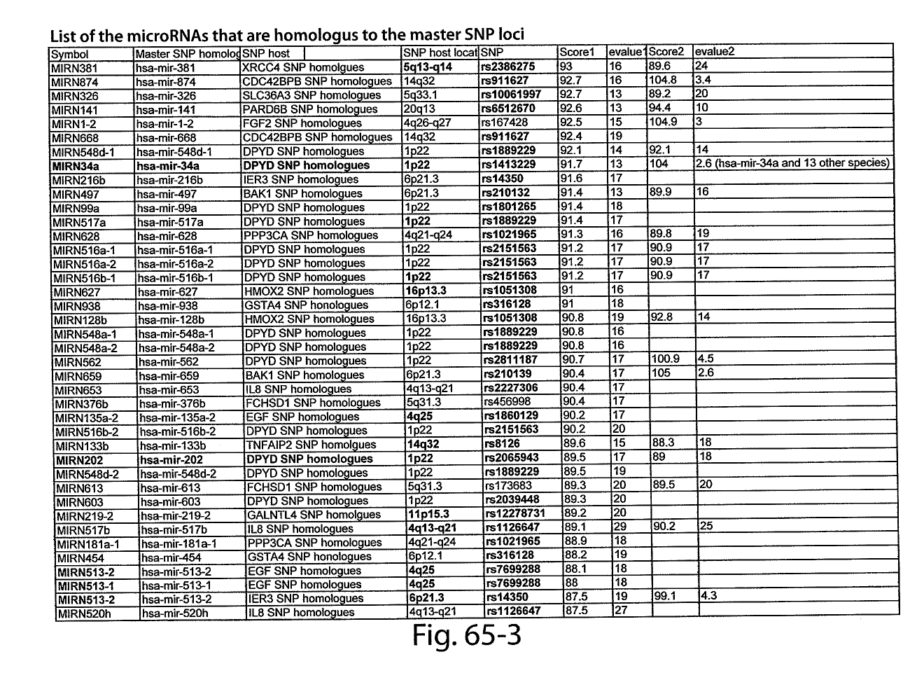

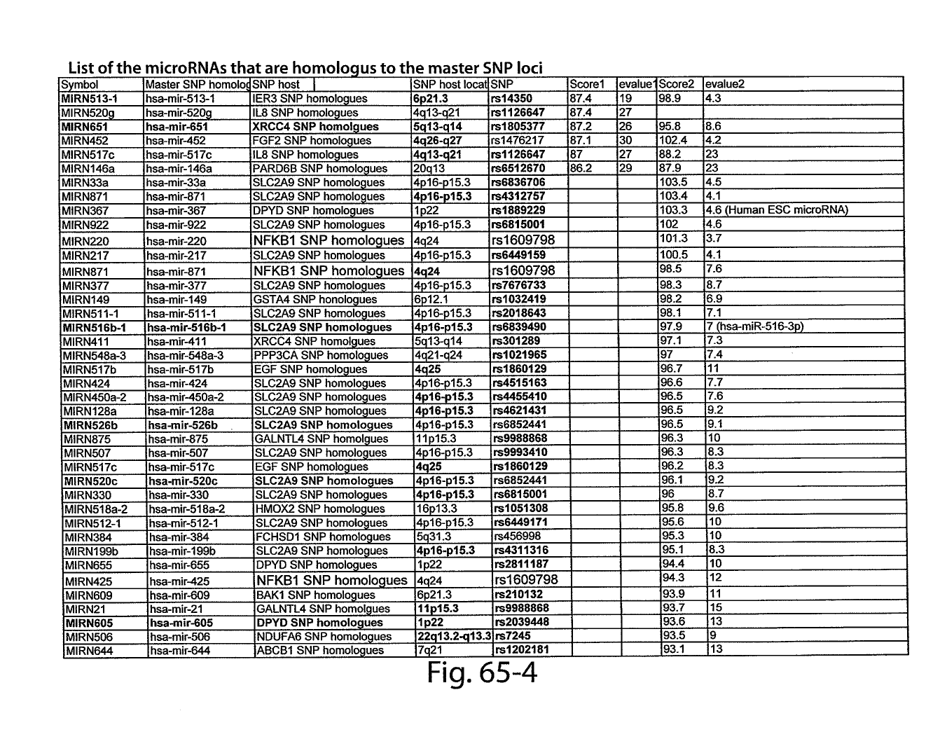

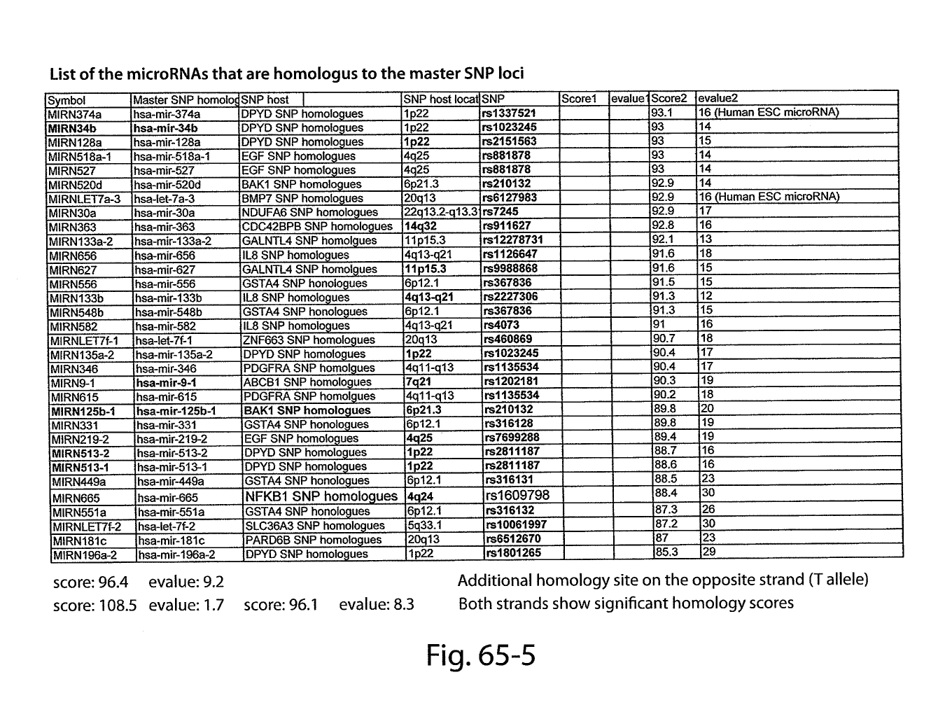

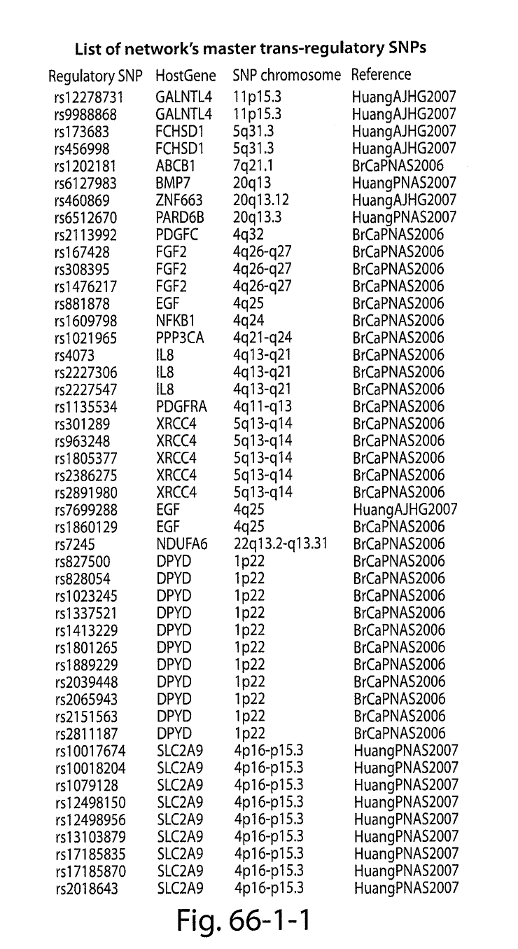

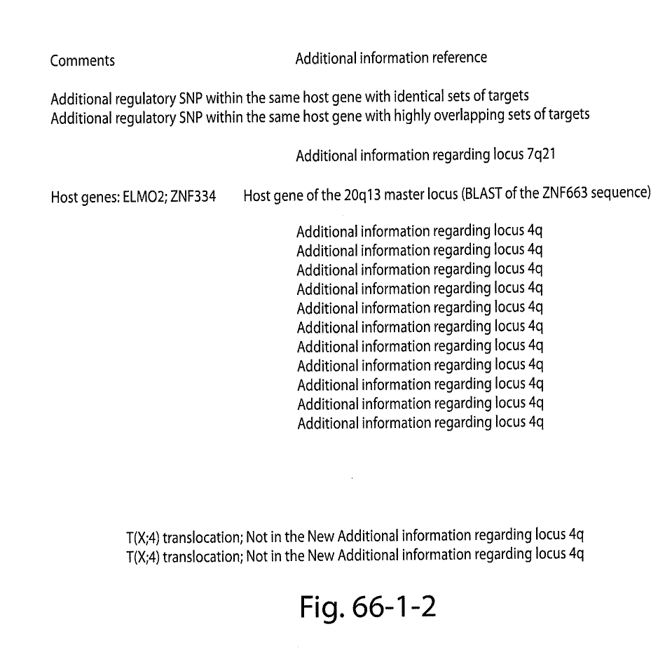

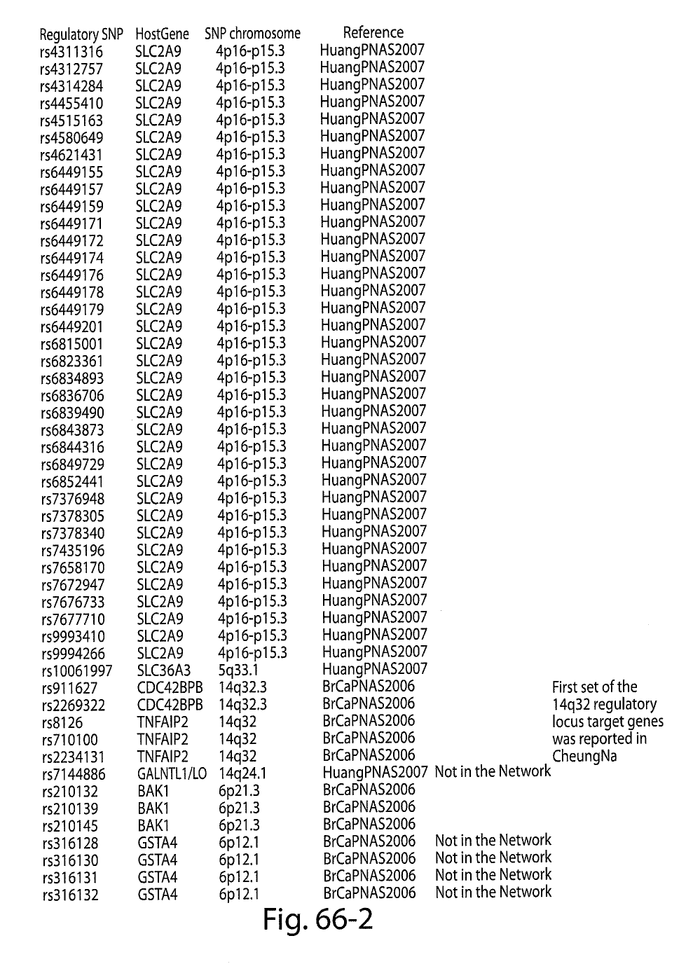

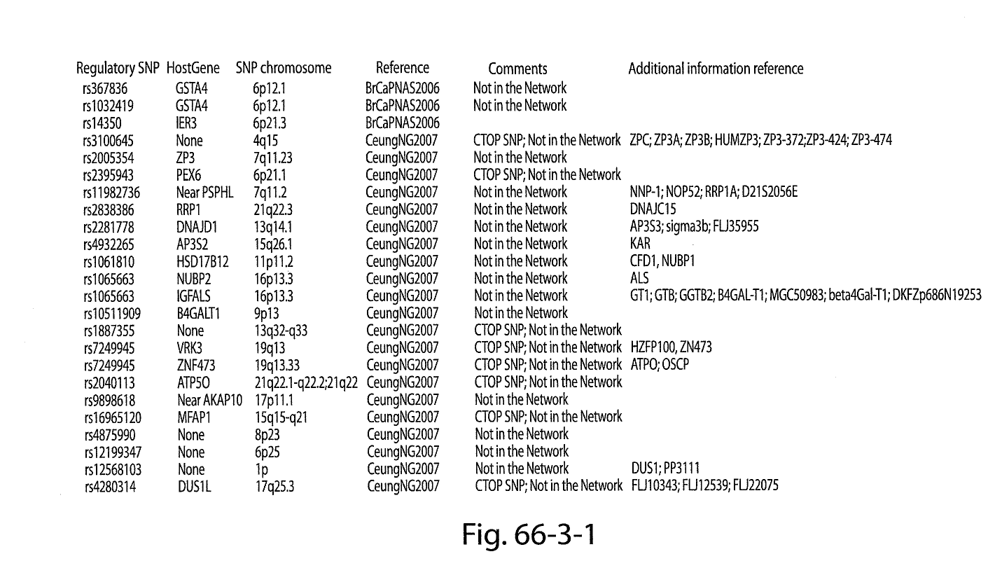

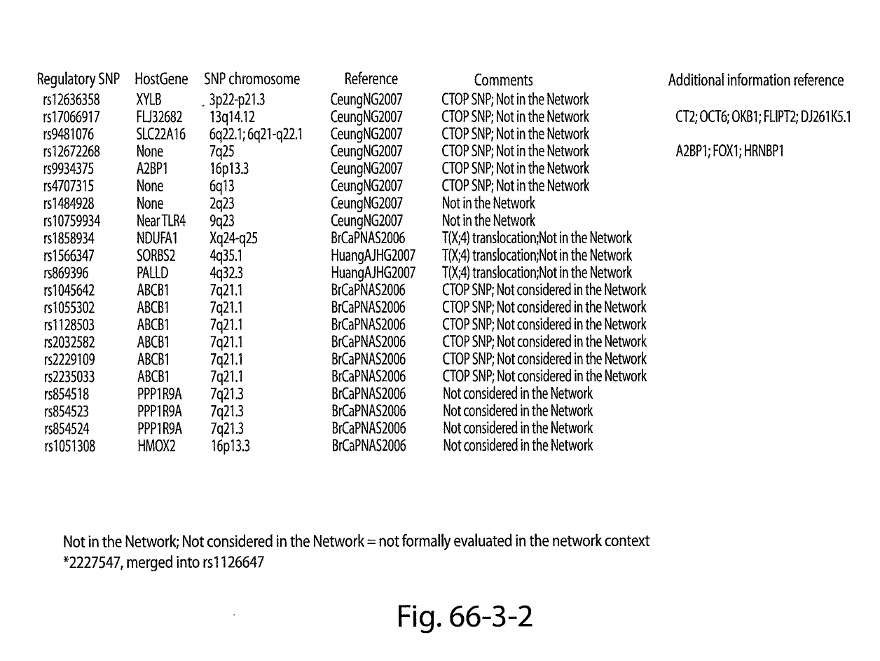

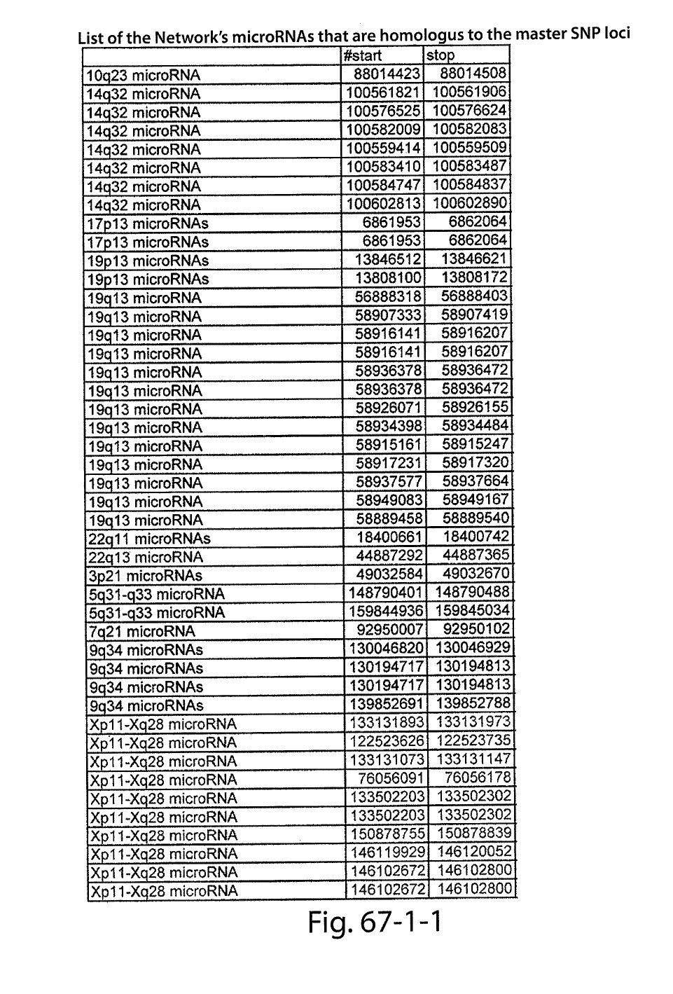

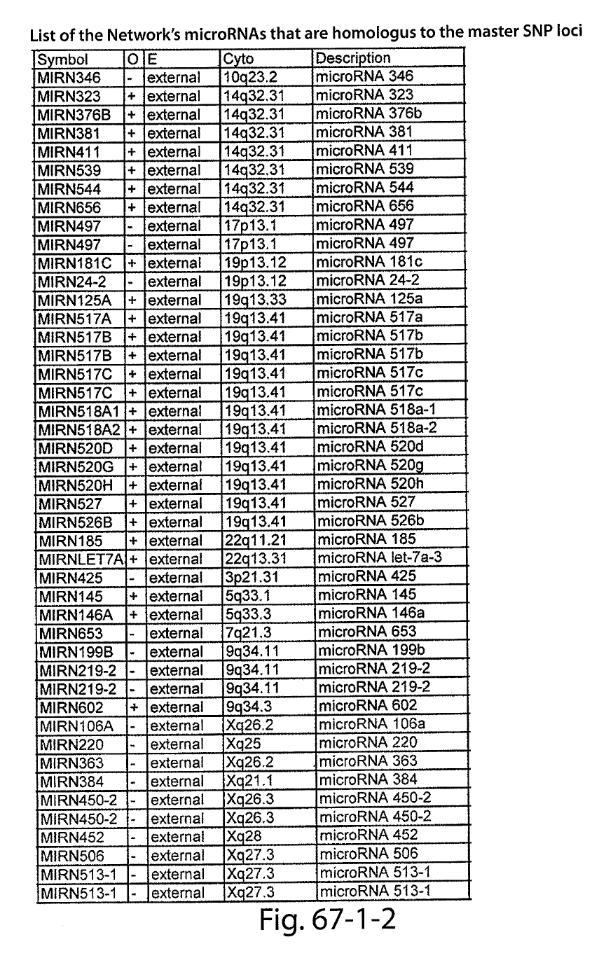

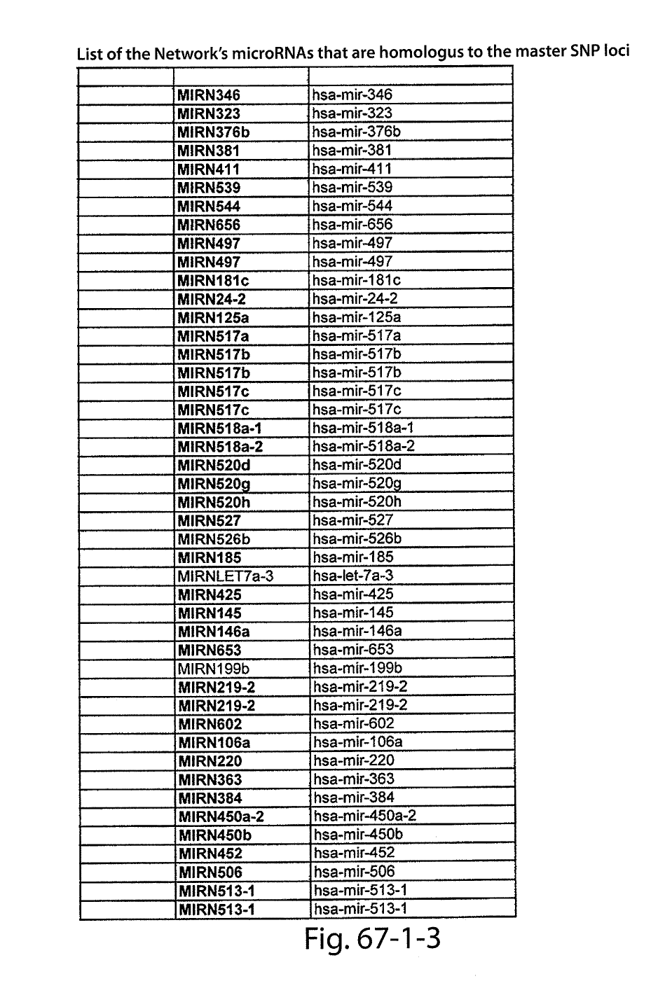

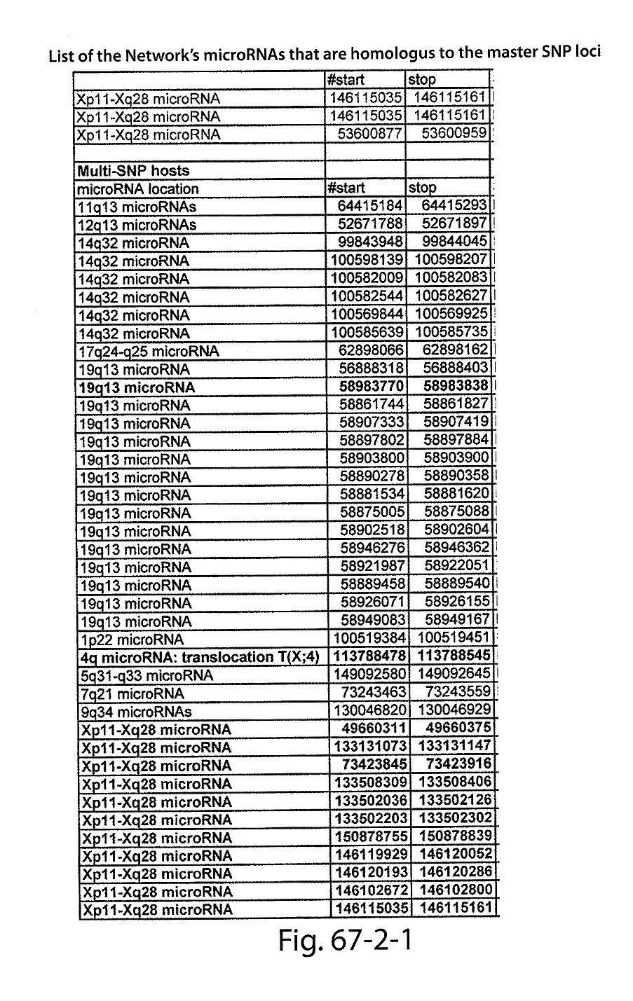

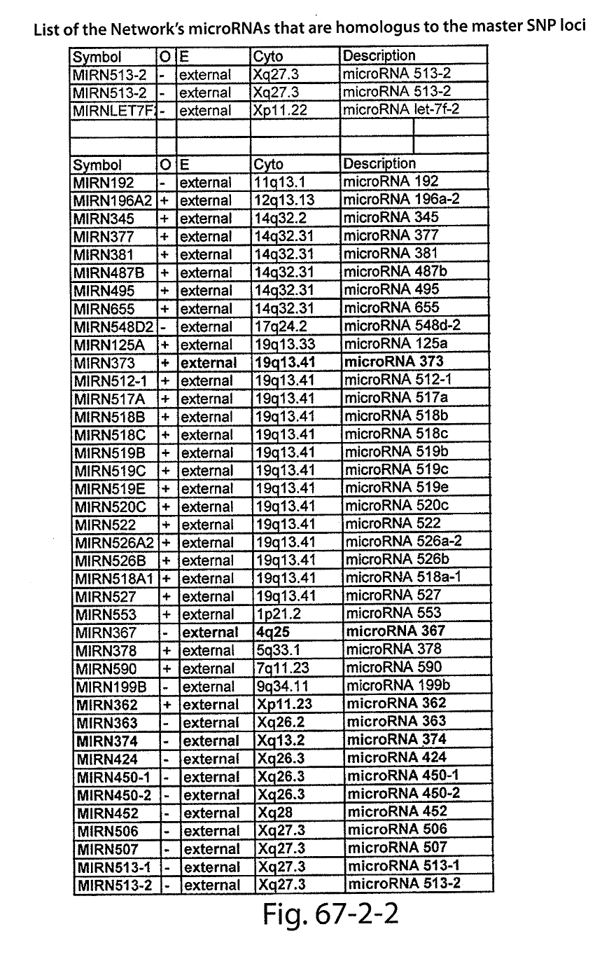

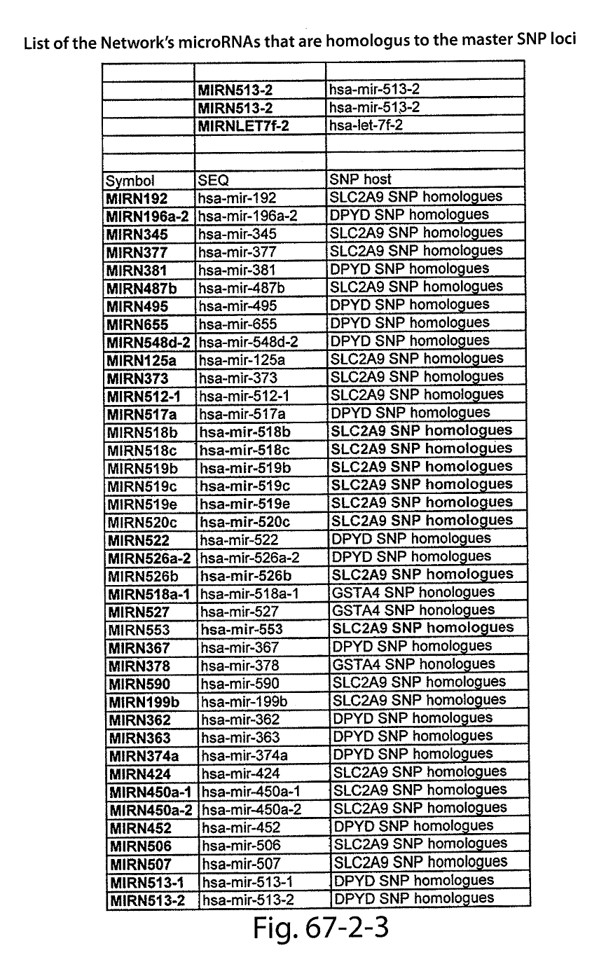

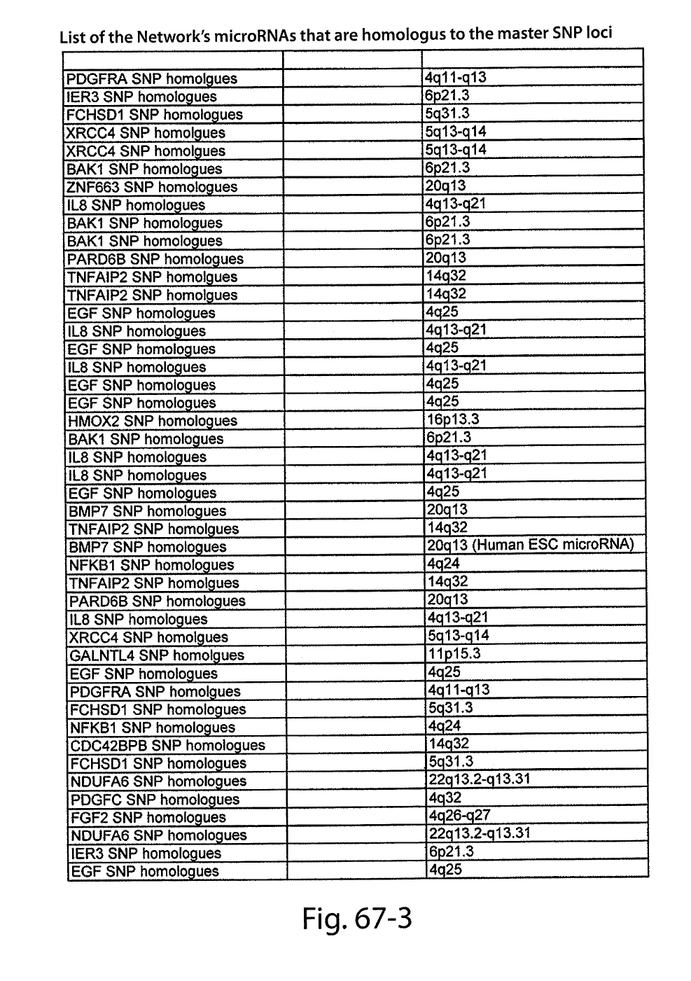

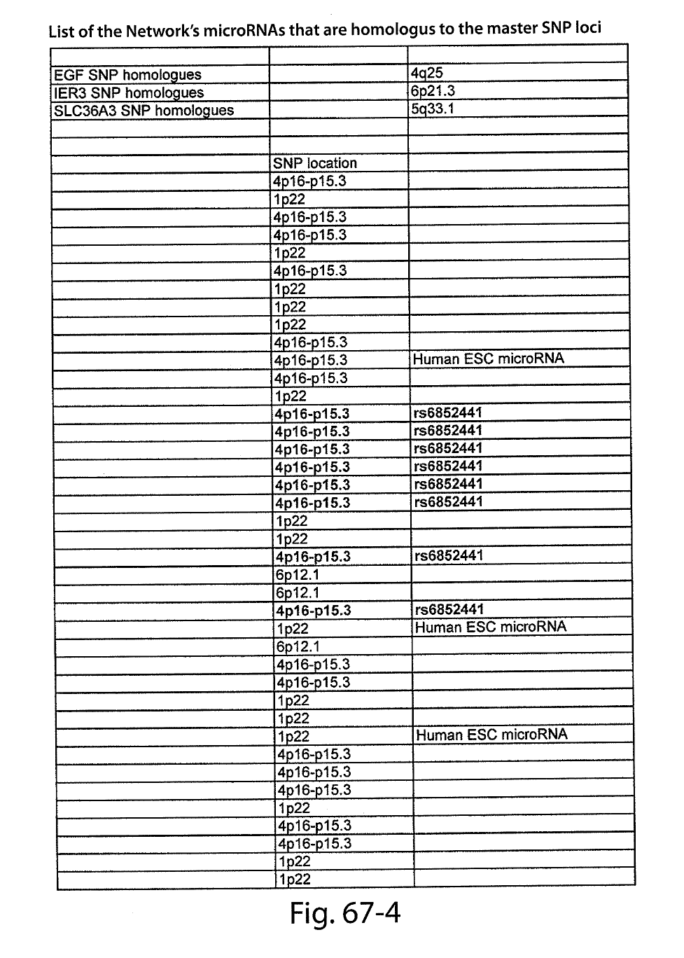

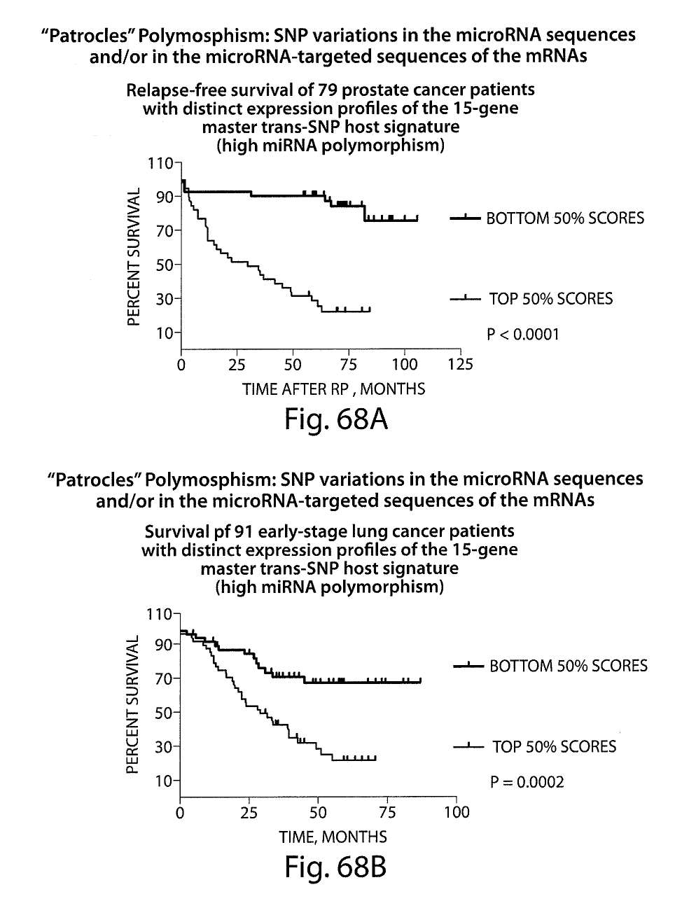

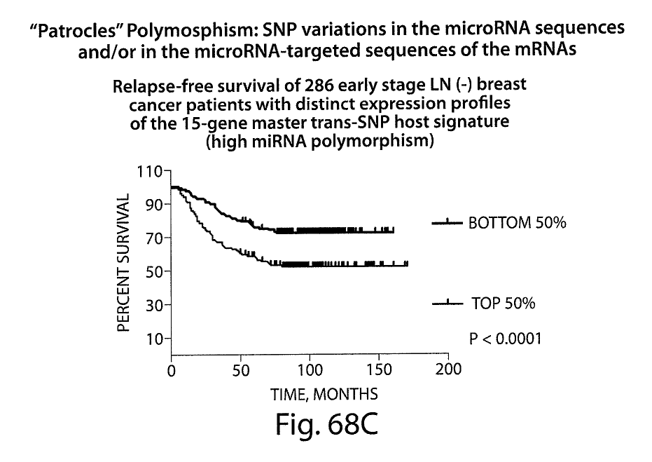

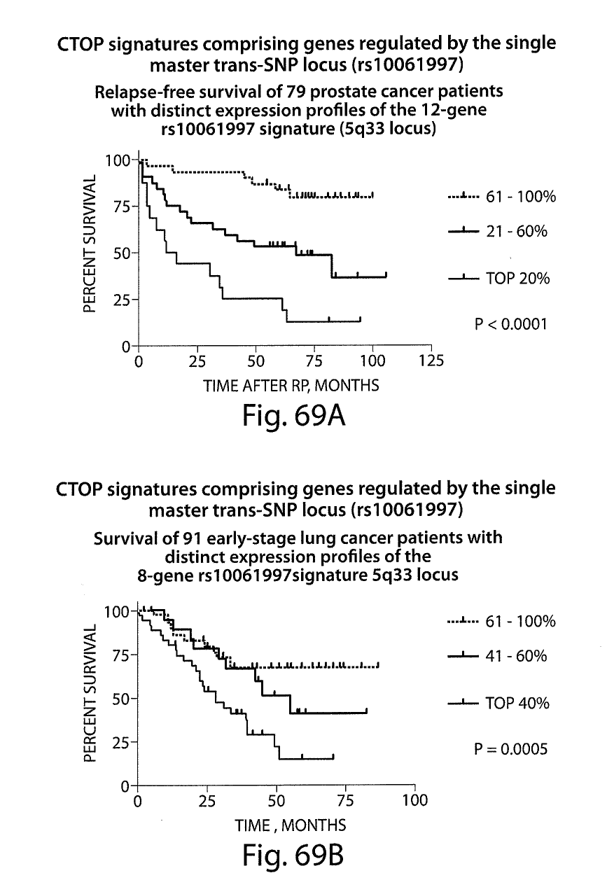

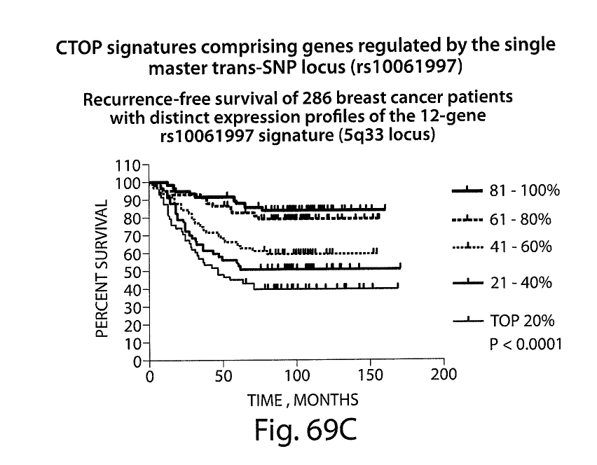

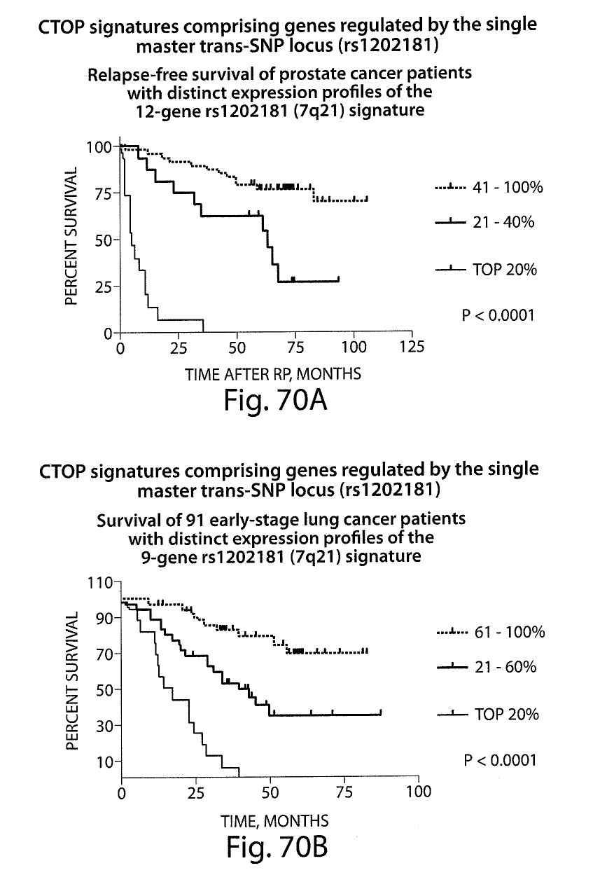

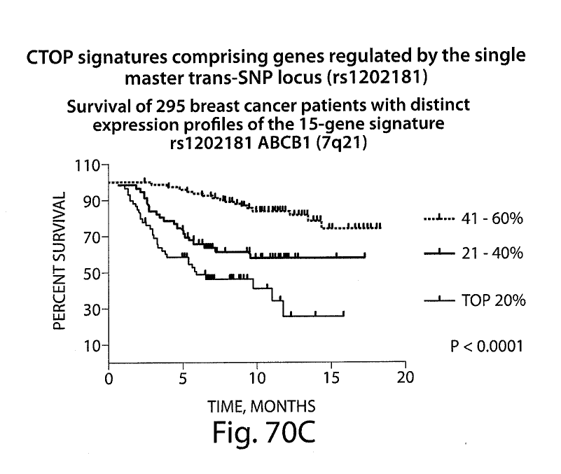

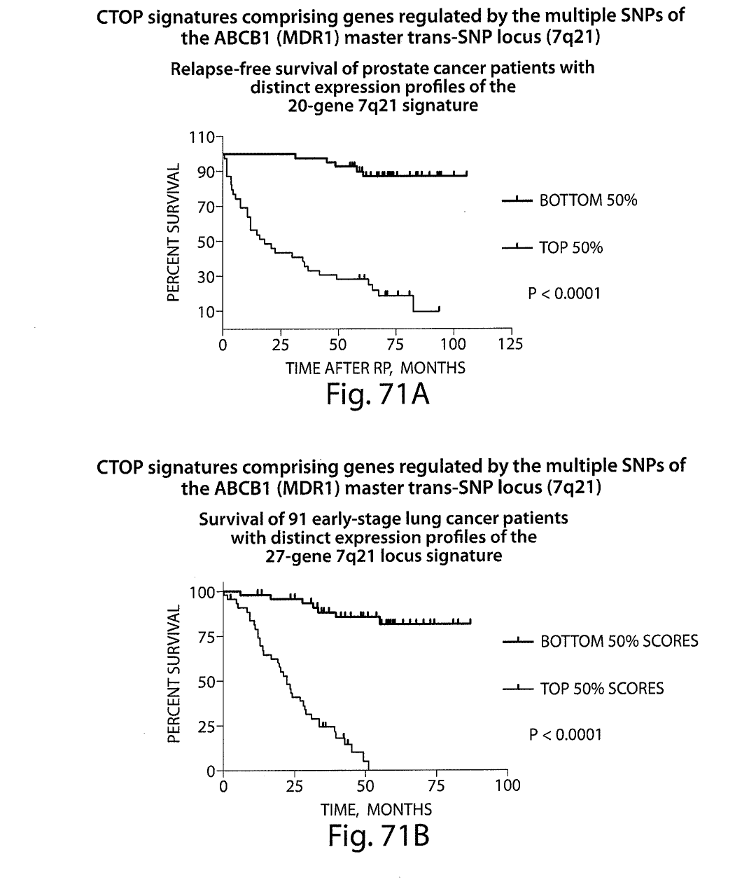

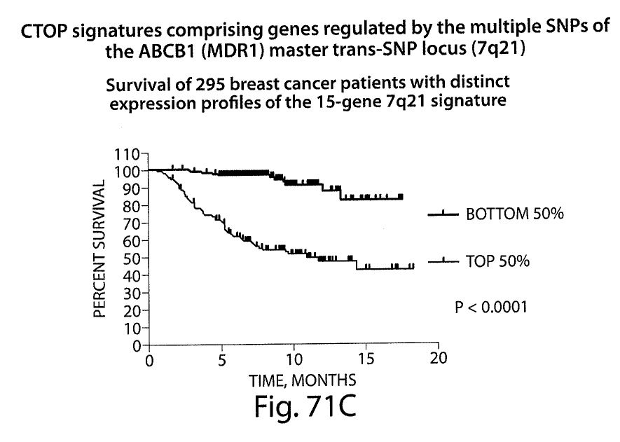

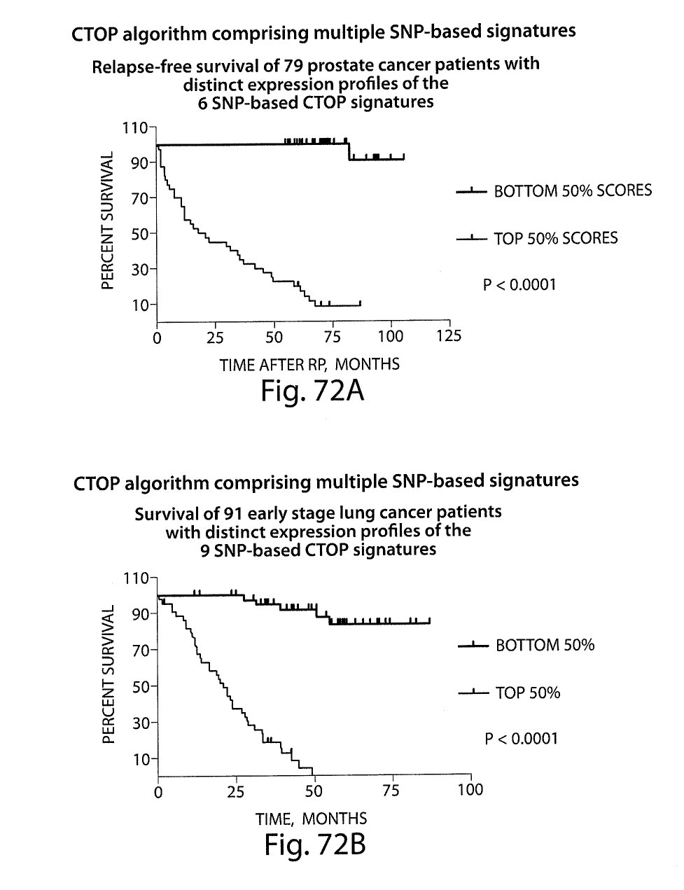

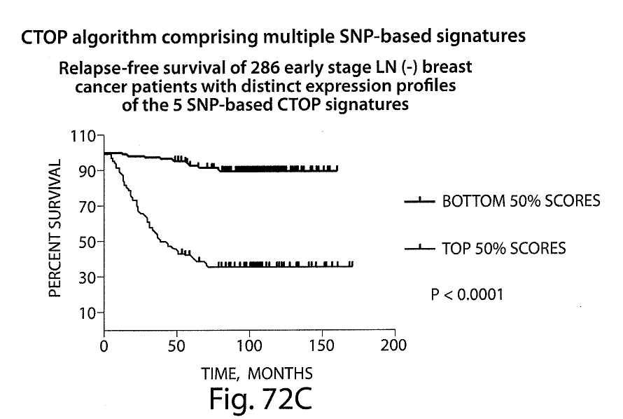

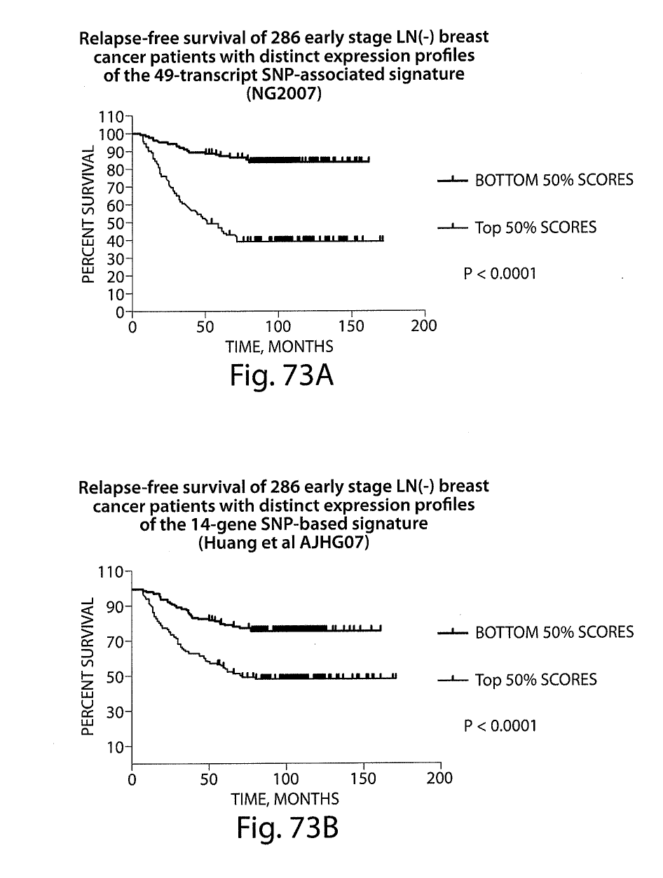

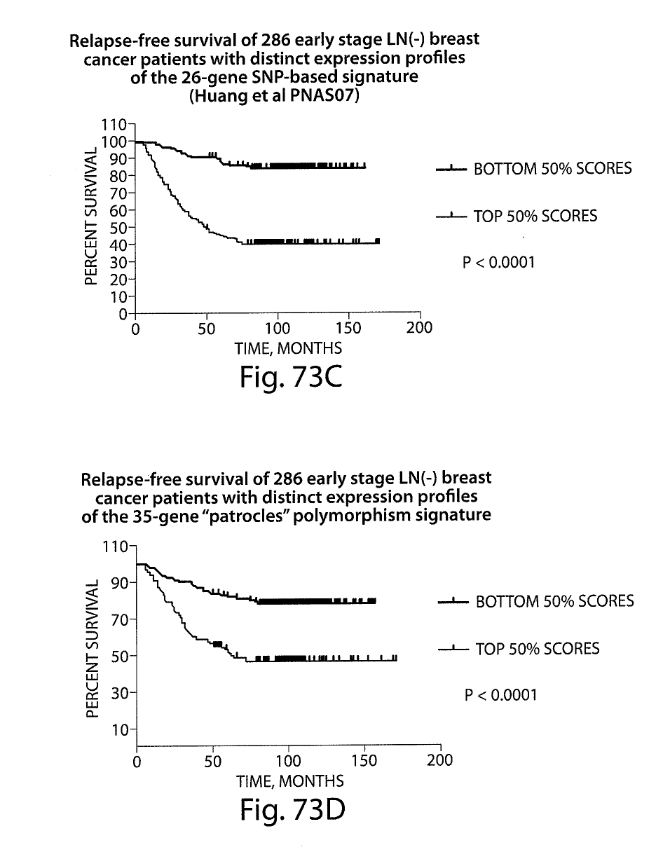

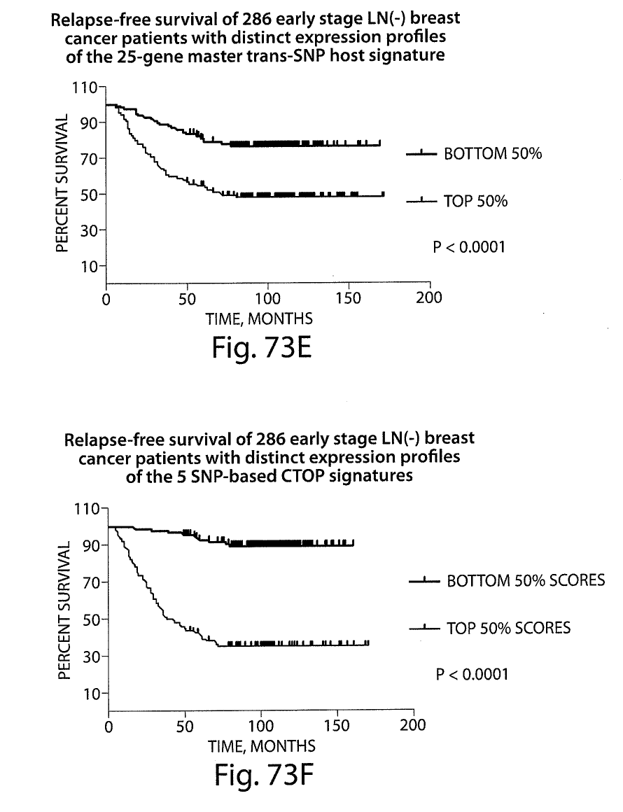

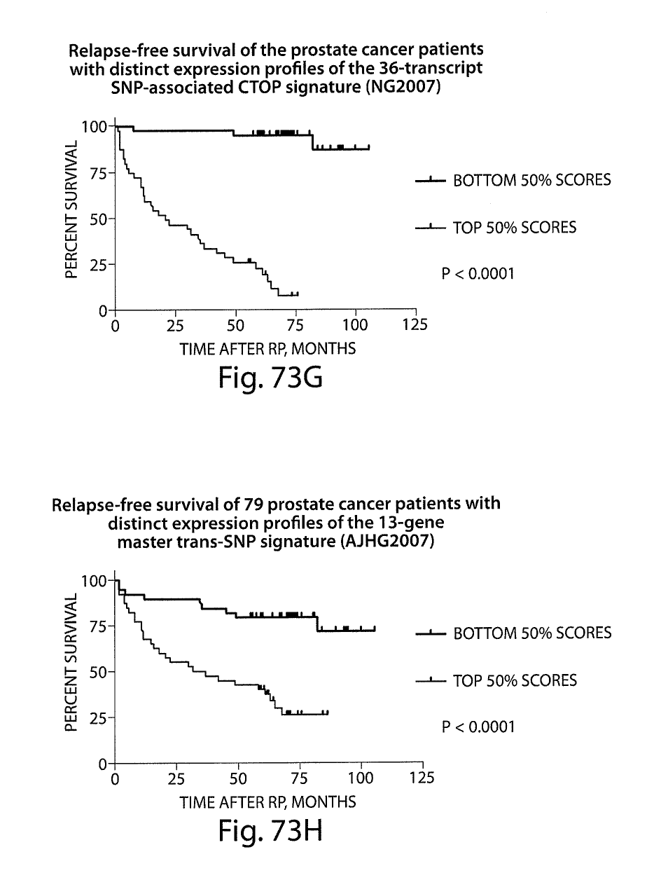

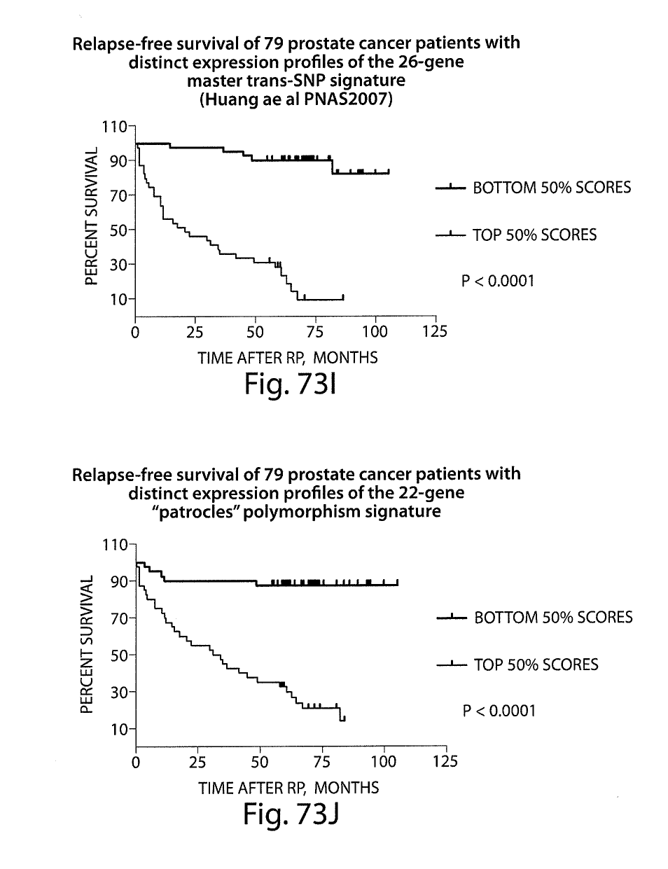

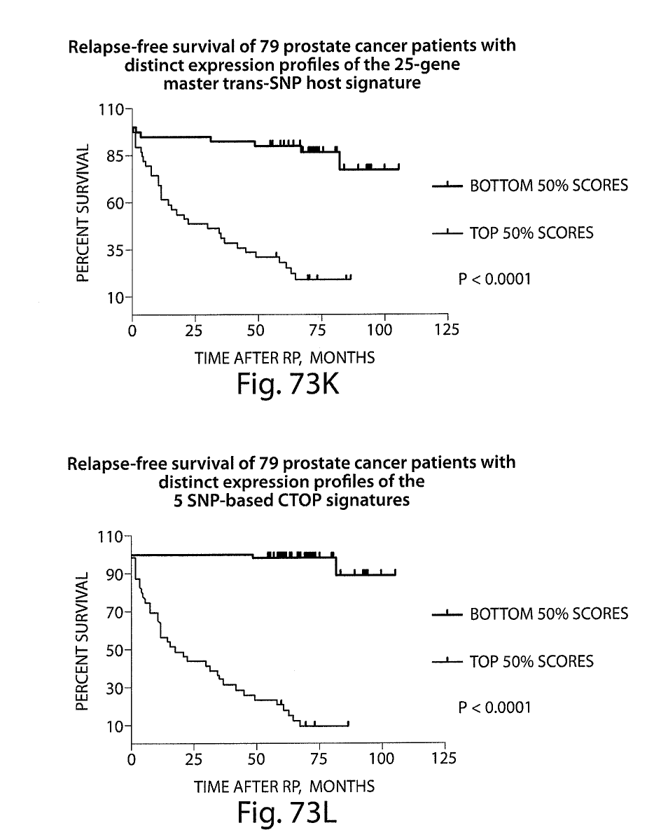

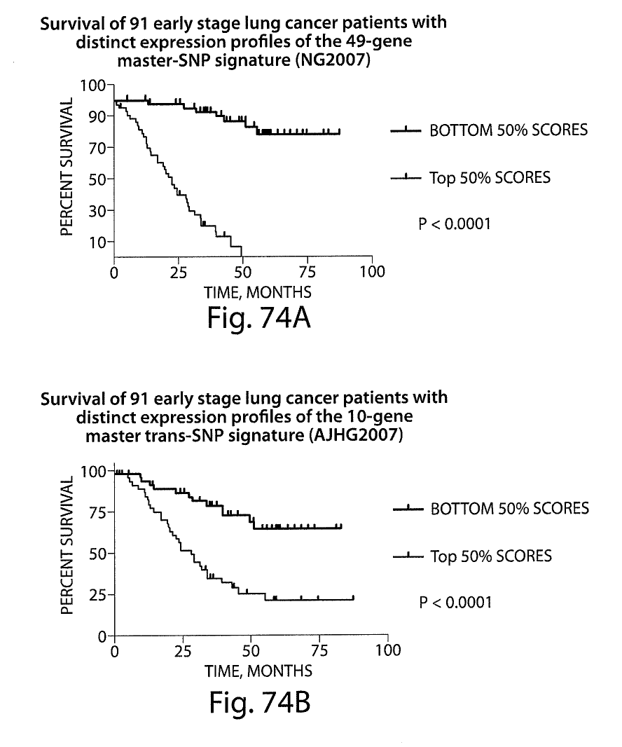

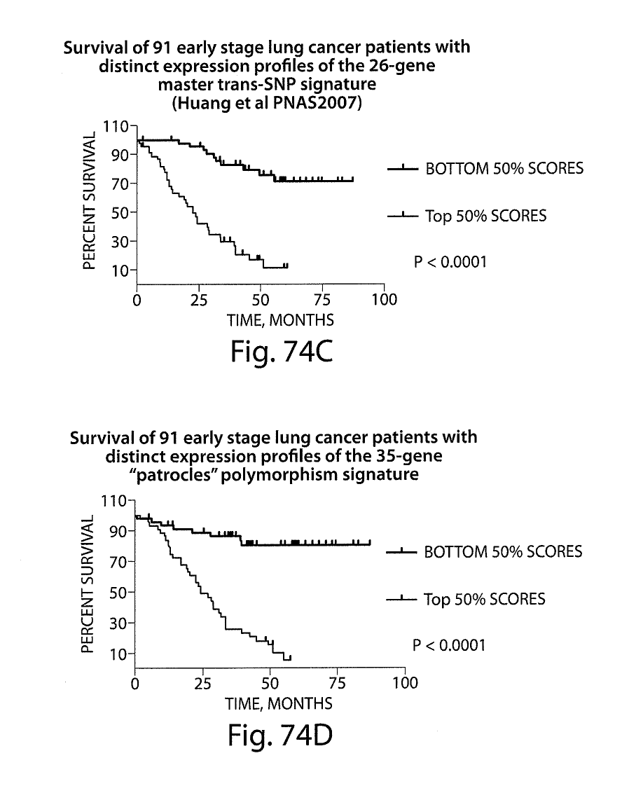

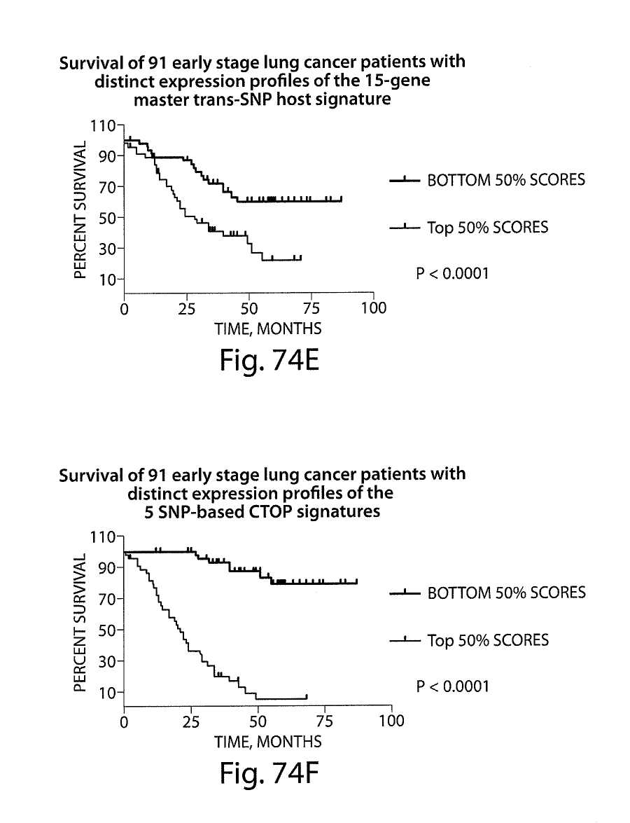

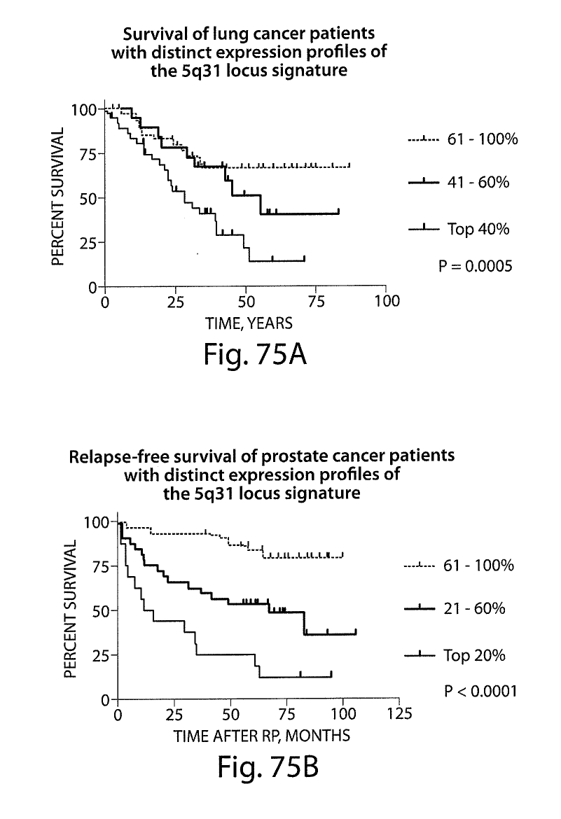

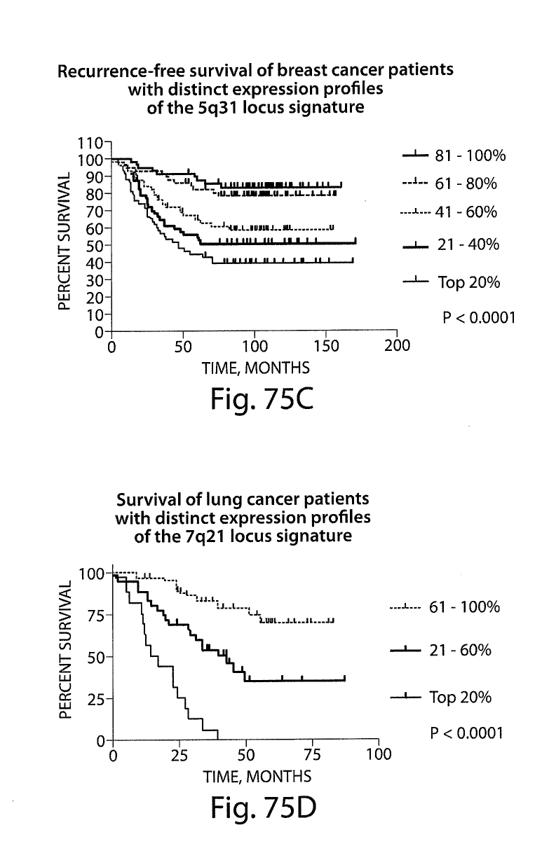

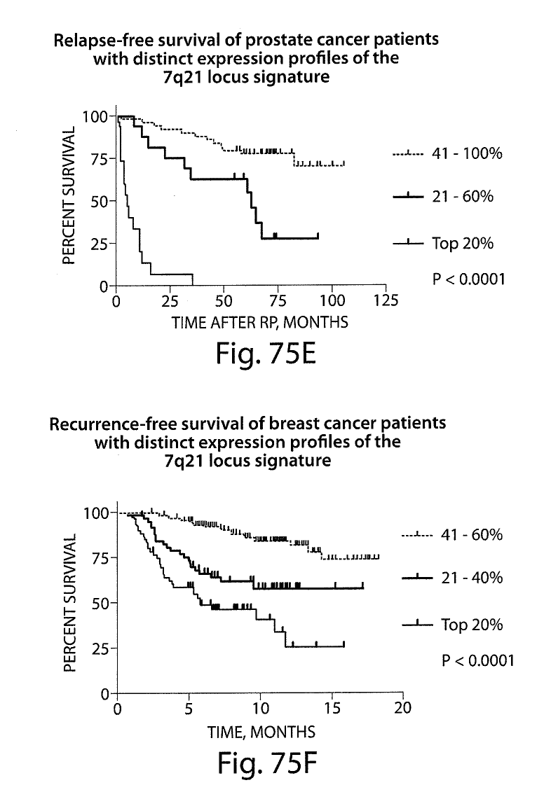

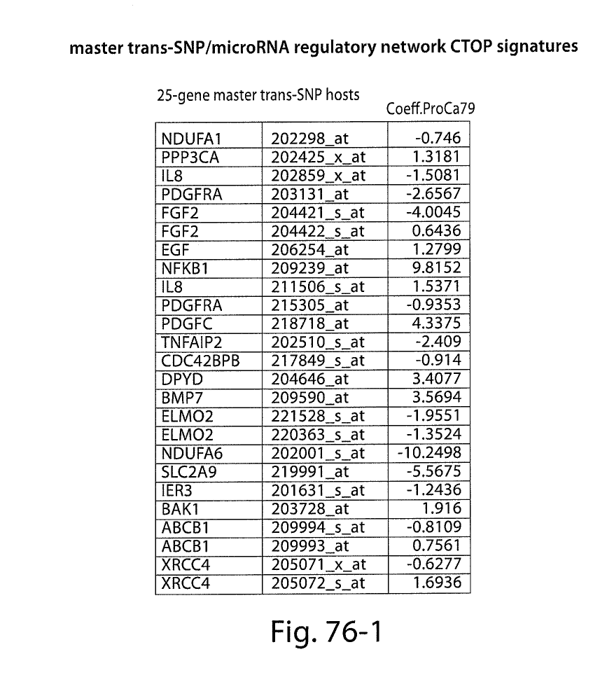

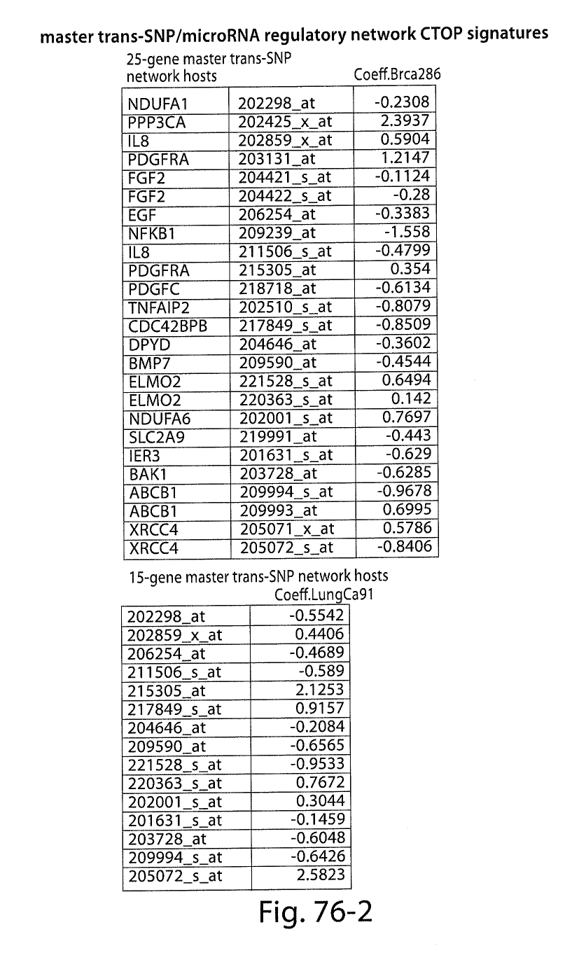

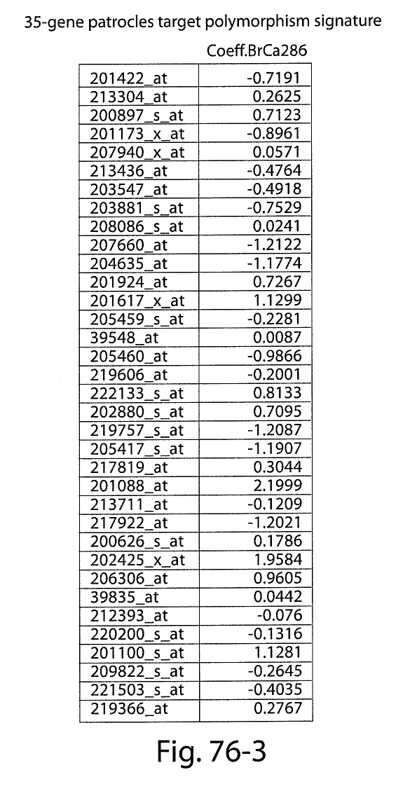

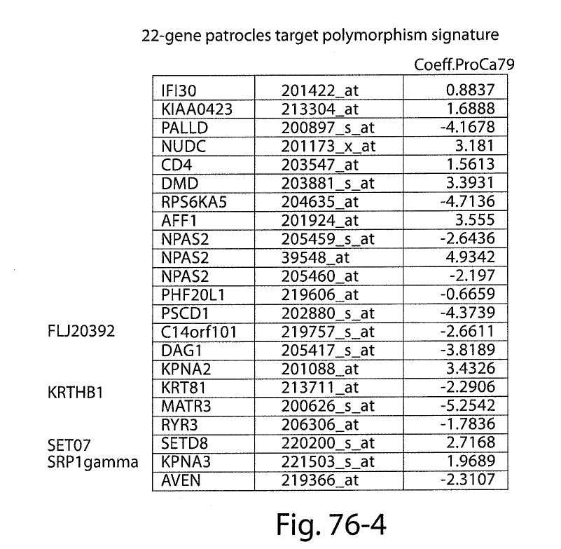

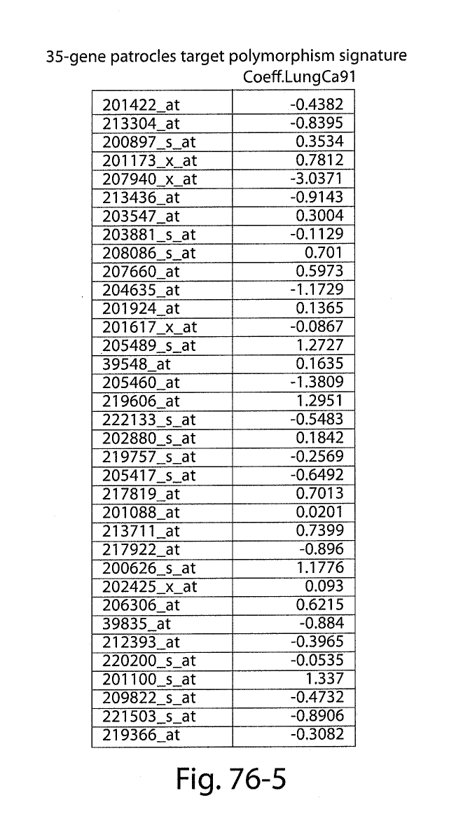

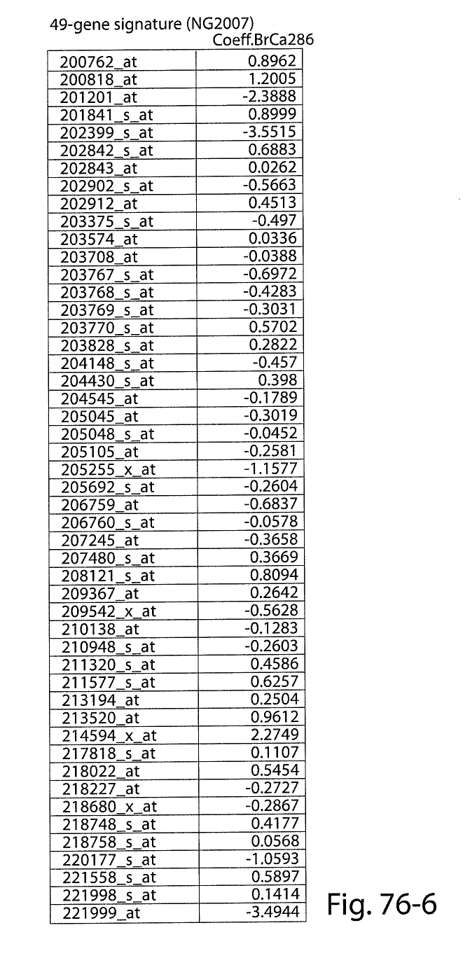

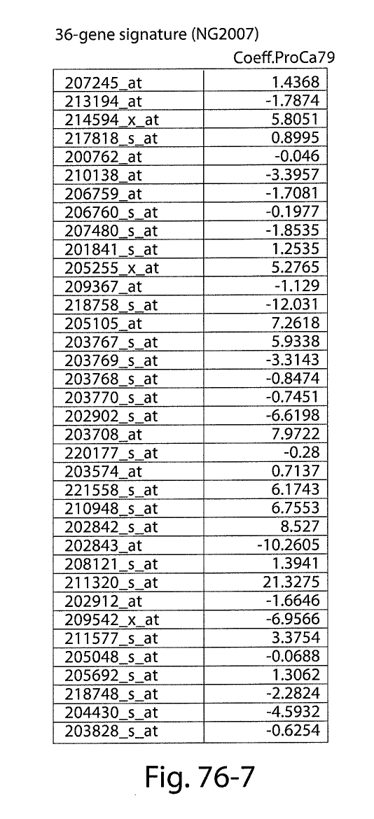

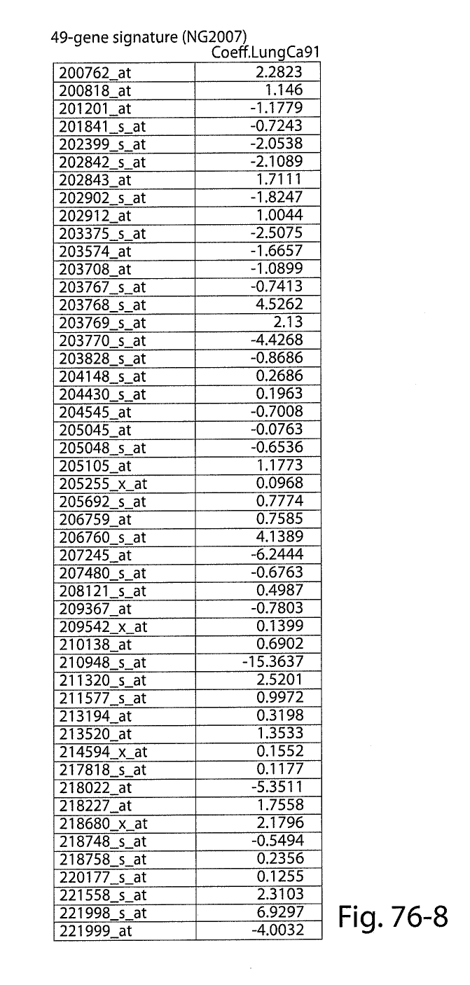

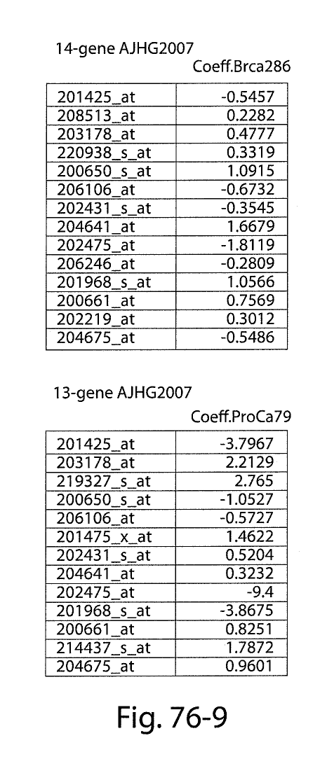

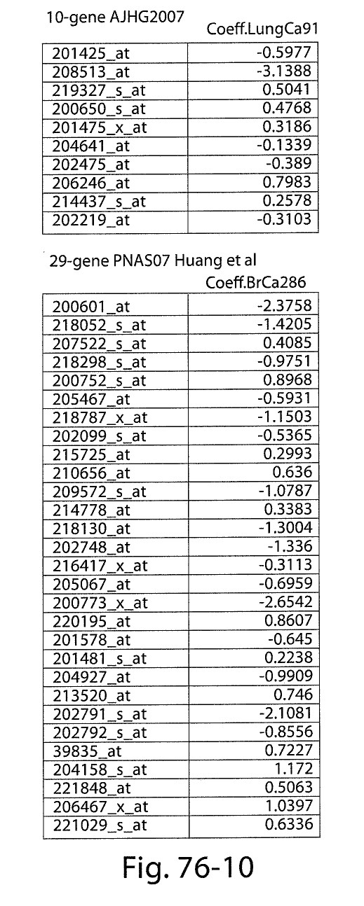

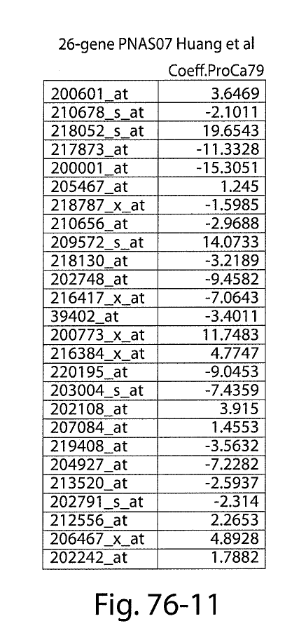

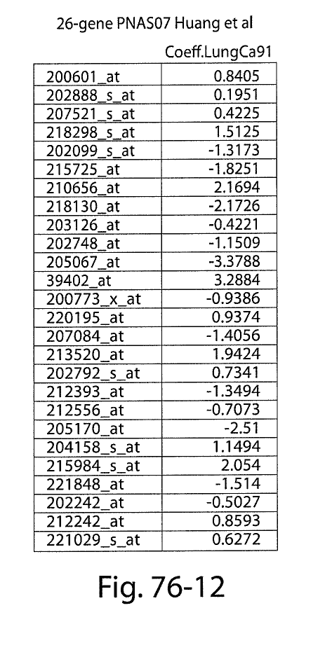

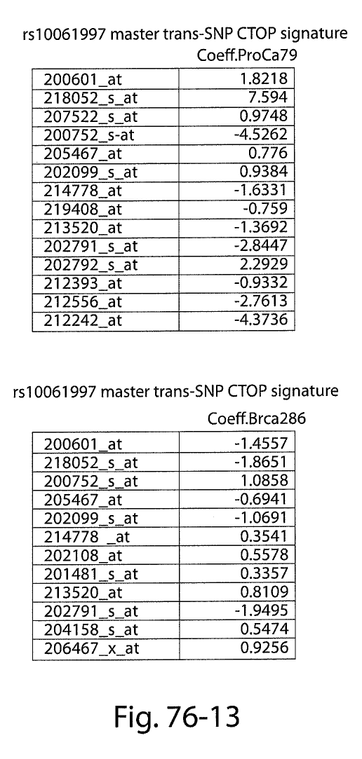

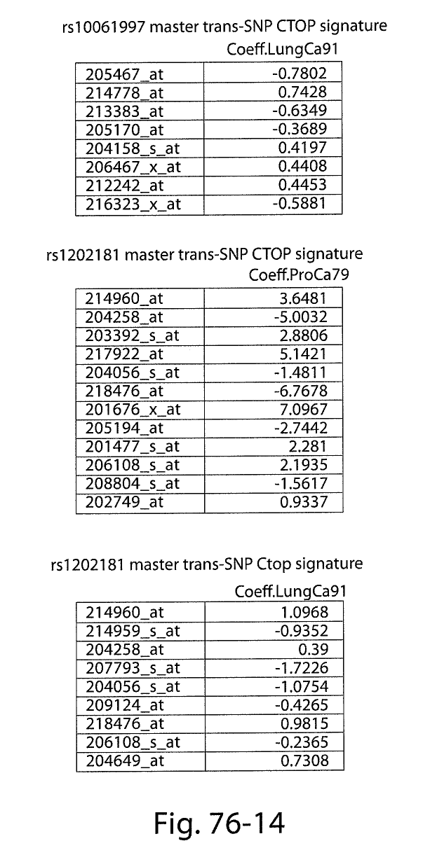

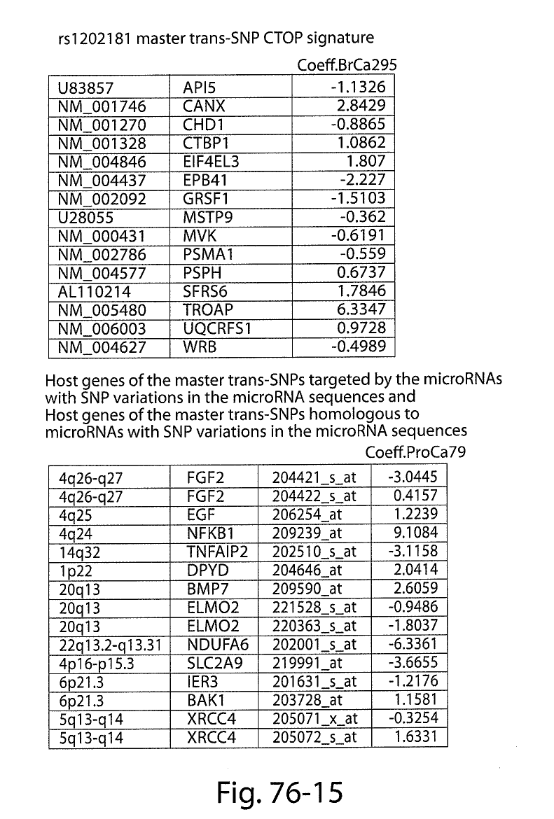

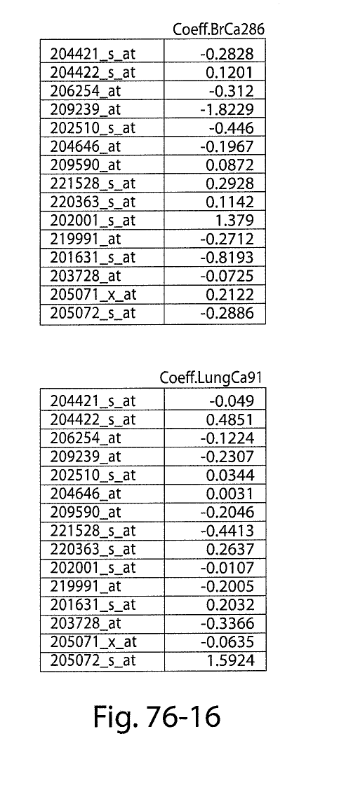

FIGS. 68-75 illustrate a practical utility of the protein-coding transcripts identified as the components of disease phenocodes mRNAs which are regulated in trans by the trans-regulatory SNPs and homologous microRNAs). The corresponding gene signatures are shown in FIGS. 64-67 and 76.

### EQUIVALENTS

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but by the claims appended hereto.

We claim:

1. A method of identifying a phenotype-linked variant genomic sequence in an individual, the method comprising
providing a genomic sequence, said genomic sequence associated with a disease or condition and containing a known sequence variation;
assessing expression of said genomic sequence; and
correlating said genomic sequence and expression to identify a variant genomic sequence whose expression is altered in a subject with a disease or condition,
thereby identifying a phenotype-linked variant genomic sequence.

2. The method of identifying a phenotype-linked variant genomic sequence in an individual of claim 1, wherein the genomic sequence is a single-nucleotide polymorphism (SNP) or a copy number variation (CNV); loss of heterozygocity (LOH); amplification; deletions; insertions; point mutations; frame-shift; duplication; epigenetic sequence modifications such as DNA methylation; epigenetic silencing or activation of transcription such modification of histone codes and nucleosomes.

3. The method of identifying a phenotype-linked variant genomic sequence in an individual of claim 1, wherein the alteration is an increase in expression compared to subject not having the disease or condition.

4. The method of identifying phenotype-linked variant genomic sequence in an individual of claim 1, wherein the alteration is a decrease in expression compared to subject not having the disease or condition.

5. The method of identifying a phenotype-linked variant genomic sequence in an individual of claim 1, further comprising the step of displaying, recording, or communicating the identified phenotype-linked variant genomic sequence.

6. The method of identifying a phenotype-linked variant genomic sequence in an individual of claim 1, further comprising the step of building a map of the identified phenotype-linked variant genomic sequence.

7. The method of identifying a phenotype-linked variant genomic sequence in an individual of claim 6, further comprising the step of using the identified phenotype-linked variant genomic sequence to identify gene expression signatures with respect to the phenotype-linked variant genomic sequence.

8. The method of identifying a phenotype-linked variant genomic sequence in an individual of claim 7, further comprising the step of selecting the phenotype-linked variant genomic sequence by cross referencing the gene expression signatures to the map of the identified phenotype-linked variant genomic sequence.

9. A method of identifying a phenocode, the method comprising:
querying a microRNA database with a variant genomic sequence whose expression is altered in a subject with a disease or condition, thereby identifying a microRNA homologous to said variant genomic sequence; and
identifying an mRNA homologous to said microRNA;
thereby identifying a phenocode comprising said variant genomic sequence, said homologous microRNA, and said mRNA.

10. The method of identifying a phenocode of claim 9, wherein the genomic sequence is a single-nucleotide polymorphism (SNP) or a copy number variation (CNV).

11. The method of identifying a phenocode of claim 9, further comprising the step of displaying said phenocode.

12. The method of identifying a phenocode of claim 9, further comprising the step of producing a sequence homology map.

13. The method of identifying a phenocode of claim 9, wherein the variant genomic sequence is a top scoring variant genomic sequence and wherein the method further comprises the step of identifying microRNAs having largest number of homology events.

14. The method of identifying a phenocode of claim 9, wherein the disease or condition is selected from the group consisting of breast cancer, prostate cancer, colorectal cancer, lung cancer, ovarian cancer, systemic lupus erythematosus, vitiligo, vitiligo-associated multiple autoimmune disease, type 2 diabetes, type 1 diabetes, Crohn's disease, coronary artery disease, hypertension, rheumatoid arthritis, bipolar disorder, ankylosing spondylitis, Graves' disease, multiple sclerosis, Huntington's disease, ulcerative colitis, Alzheimer's, autism; autoimmune thyroid disease, schizophrenia, ageing and centenarians phenotypes.

15. The method of identifying a phenocode of claim 9, further comprising the step of identifying those mRNAs that are encoded by protein-coding genes and assessing the expression of identified mRNAs.

16. The method of identifying a phenocode of claim 15, wherein the protein-coding gene is part of the nuclear import pathway or the inflammasome pathway.

17. The method of identifying a phenocode of claim 16, wherein the protein-coding gene is selected from the group consisting of KPNA1, NLRP1, NLRP3, HLA-DRB1, PTPN22, OLIG3/TNFAIP3, STAT4, TRAF1/C5, ACAN, WNT5A, MMP14, HOXA11, EN1, DICER1, TSC1, MYB, MYBL1, HMGA1, ACAN, CEBPB, PTEN and combinations thereof.

18. The method of identifying a phenocode of claim 15, wherein KPNA1 expression is altered.

19. The method of identifying a phenocode of claim 9, wherein the identified microRNA is homologous to the variant genomic sequence whose expression is altered in the subject with the disease or condition.

20. The method of identifying a phenocode of claim 19, wherein the identified microRNA targets one or more protein-coding mRNAs in the nuclear import pathway or the inflammasome pathway.

21. A computer-readable medium comprising computer executable instructions recorded thereon for performing the method comprising:
querying a microRNA database with a variant genomic sequence whose expression is altered in a subject with a disease or condition to identify a microRNA homologous to said variant genomic sequence.

22. The computer-readable medium of claim 21, wherein the method further comprises the step of identifying an mRNA homologous to said microRNA, thereby obtaining a phenocode comprising said variant genomic sequence, said homologous microRNA, and said mRNA and displaying said phenocode on the computer-readable medium.

23. A method of reversing a disease or condition associated with altered gene expression phenotypes of the nuclear import or inflammasome pathways comprising administering an effective amount of a pharmaceutical compound to a subject, wherein, following administration of the pharmaceutical compound, the alteration of gene expression is reversed in the subject.

24. The method of reversing a disease associated with altered gene expression phenotypes of nuclear import or inflammasome pathways of claim 23, wherein the pharmaceutical compound is chloroquine or rapamycin.

25. The method of reversing a disease associated with altered gene expression phenotypes of nuclear import and inflammasome pathways of claim 23, wherein the gene whose expression is altered is one or more of the KPNA1, NLRP1, and NLRP3 genes.

26. An apparatus for evaluating a disease or a risk of disease in a patient, the apparatus comprising:
a model predictive of a disease phenocode configured to evaluate a dataset for the patient to thereby evaluate the risk of disease in said patient, wherein the model is based on a set of disease-linked SNPs,
microRNAs displaying sequence homology or complementarity to the disease-linked SNPs,
and mRNAs encoded by protein-coding genes,
wherein said mRNAs are targeted by said microRNAs, wherein the disease-linked SNPs exert a regulatory effect in trans.

27. The apparatus for evaluating a disease or a risk of disease in a patient of claim 26, wherein the disease is selected from the group consisting of breast cancer, prostate cancer, colorectal cancer, lung cancer, ovarian cancer, systemic lupus erythematosus, vitiligo, vitiligo-associated multiple autoimmune disease, type 2 diabetes, type 1 diabetes, Crohn's disease, coronary artery disease, hypertension, rheumatoid arthritis, bipolar disorder, ankylosing spondylitis, Graves' disease, multiple sclerosis, Huntington's disease, ulcerative colitis, Alzheimer's, autism; autoimmune thyroid disease, schizophrenia, ageing and centenarians phenotypes.

28. A method of screening for candidate compounds capable of reversing a disease or condition associated with an altered gene expression phenotypes of the nuclear import or inflammasome pathways, the method comprising:
a) detecting the level of gene expression in a subject administered a candidate compound, wherein said subject is suffering from said disease or condition;
b) comparing the level of gene expression for the candidate compound with that of a reference compound known to reverse the altered gene expression associated with the disease or condition; and
c) determining the differences, if any, between the levels of gene expression for the candidate compound and the reference compound,
thereby identifying whether the candidate compound is capable of reversing the disease or condition.

29. A method of determining susceptibility to a disease or condition in a subject, the method comprising
a) determining for said subject a disease phenocode, wherein said phenocode comprises:
(i) a set of disease-linked SNPs,
(ii) microRNAs displaying sequence homology or complementarity to the disease-linked SNPs, and
(iii) mRNAs encoded by protein-coding genes,
wherein said mRNAs are targeted by said microRNAs, and wherein the disease-linked SNPs exert a regulatory effect in trans; and
b) assessing susceptibility to said disease in said subject based on said phenocode.

30. A method of assessing prognosis of a disease or condition in a subject, the method comprising:
a) determining for said subject a disease phenocode, wherein said phenocode comprises:
(i) a set of disease-linked SNPs,
(ii) microRNAs displaying sequence homology or complementarity to the disease-linked SNPs, and
(iii) mRNAs encoded by protein-coding genes,
wherein said mRNAs are targeted by said microRNAs, and wherein the disease-linked SNPs exert a regulatory effect in trans; and
b) assessing prognosis of said disease based on said phenocode.

31. The method of assessing prognosis of a disease or condition in a subject of claim 30, wherein the method is performed in computer system such that a reported analysis for said phenocode is presented on a display.

32. The method of assessing prognosis of a disease or condition in a subject of claim 31, wherein the reported analysis is stored in a computer-readable medium.

33. The method of assessing prognosis of a disease or condition in a subject of claim 30, wherein said phenocode is determined on a computer.

34. The method of assessing prognosis of a disease or condition in a subject of claim 30, wherein said phenocode is displayed on a readable device.

35. A method of assessing the risk of a developing disease or condition, or of having a predisposition to develop a disease or condition in an individual, the method comprising assessing the status of one or more molecular components of a phenocode identified according to the method of claim 9.

36. A method of identification of therapeutic compounds, preventive compounds or both by assessing the effect of one or more test compounds on profiles of one or more molecular components of a disease phenocode identified according to the method of claim 9 and selecting those compounds causing the reversal of said profiles.
